(19) United States
(12) Patent Application Publication
(10) Pub. No.: US 2023/0257396 A1
(43) Pub. Date: Aug. 17, 2023

(54) MACROCYCLES AND THEIR USE

(71) Applicant: BLOSSOMHILL THERAPEUTICS, INC., San Diego, CA (US)

(72) Inventor: Jingrong Jean CUI, San Diego, CA (US)

(21) Appl. No.: 18/004,829

(22) PCT Filed: Jul. 8, 2021
(86) PCT No.: PCT/US21/40859

§ 371 Date: Jan. 9, 2023

Related U.S. Application Data
(60) Provisional application No. 63/050,559, filed on Jul. 10, 2020.
(60) Provisional application No. 63/143,569, filed on Jan. 29, 2021.
(60) Provisional application No. 63/217,950, filed on Jul. 2, 2021.

(51) Int. Cl. C07D 498/22; C07D 487/22; C07D 515/18; C07D 498/18

(52) U.S. Cl. CPC C07D 498/22; C07D 487/22; C07D 515/18; C07D 498/18

(57) ABSTRACT

The present disclosure relates to macrocyclic compounds, pharmaceutical compositions containing macrocyclic compounds, and methods of using macrocyclic compounds to treat disease, such as cancer.

### CROSS-REFERENCE TO RELATED APPLICATIONS

This application claims priority under 35 U.S.C. § 119(e) to U.S. Provisional Application Ser. No. 63/050,559 filed on Jul. 10, 2020, U.S. Provisional Application Ser. No. 63/143,569 filed on Jan. 29, 2021, and U.S. Provisional Application Ser. No. 63/217,950 filed on Jul. 2, 2021, the entire disclosures of all of which are incorporated herein by reference.

### TECHNICAL FIELD

The present disclosure relates to macrocyclic compounds, pharmaceutical compositions containing macrocyclic compounds, and methods of using macrocyclic compounds to treat disease, such as cancer.

### BACKGROUND

Protein kinases are tightly regulated signaling proteins that orchestrate the activation of signaling cascades by phosphorylating target proteins in response to extracellular and intracellular stimuli. The human genome encodes approximately 518 protein kinases (Manning G, et al The protein kinase complement of the human genome. Science. 2002, 298:1912-34). Dysregulation of kinase activity is associated with many diseases, including cancers, and cardiovascular, degenerative, immunological, infectious, inflammatory, and metabolic diseases (Levitzki, A. Protein kinase inhibitors as a therapeutic modality. Acc. Chem. Res. 2003, 36:462-469). The molecular bases leading to various diseases include kinase gain- and loss-of-function mutations, gene amplifications and deletions, splicing changes, and translocations (Wilson L J, et A New Perspectives, Opportunities, and Challenges in Exploring the Human Protein Kinome. Cancer Res. 2018, 78:15-29). The critical role of kinases in cancer and other diseases makes them attractive targets for drug inventions with 52 small molecule kinase inhibitors have been approved and 46 of them for cancer targeted therapies (Roskoski R Jr, Properties of FDA-approved Small Molecule Protein Kinase Inhibitors: A 2020 Update. Pharmacol Res 2020, 152:104609). Although kinase inhibitors have achieved dramatical success in cancer targeted therapies, the development of treatment resistance has remained as a challenge for small molecule kinase inhibitors. Acquired secondary mutations within kinase domain during the treatment often lead to treatment resistance to kinase inhibitors (Pottier C, et al Tyrosine Kinase Inhibitors in Cancer: Breakthrough and Challenges of Targeted Therapy. Cancers (Basel), 2020, 12:731). Therefore, it is necessary to invent kinase inhibitors that can target not only the kinase oncogenic drivers, and also overcome most frequent resistance mutations for better efficacy and longer disease control.

Non-small-cell lung cancer (NSCLC) is the leading cause of cancer mortality worldwide (World Health Organisation. Cancer Fact Sheet 2017). Activating EGFR mutations have been reported in approximately 10% to 15% of cases of adenocarcinoma in white patients and 50% of cases in Asian patients (Chan B A, Hughes B G. Targeted therapy for non-small cell lung cancer: current standards and the promise of the future. Transl Lung Cancer Res 2015; 4:36-54). The two most frequent EGFR alterations found in NSCLC tumors are short in-frame deletions in exon 19 (del19) of the EGFR gene and L858R, a single missense mutation in exon 21 (Konduri K. et al. EGFR Fusions as Novel Therapeutic Targets in Lung Cancer. Cancer Discovery 2016, 6:601-11). The first-generation reversible EGFR inhibitors, erlotinib and gefitinib are superior to chemotherapy in patients with advanced EGFR mutation-positive (Del19 or L858R) NSCLC and have been used as first-line standard of care in this setting. However, most patients will develop resistance to gefitinib or erlotinib with 50% to 70% of tumors developing EGFR T790M gatekeeper mutation with time of treatment (Sequist L V, et al. Genotypic and histological evolution of lung cancers acquiring resistance to EGFR inhibitors. Sci Transl Med 2011; 3:75ra26).

The second generation of EGFR inhibitors afatinib and dacomitinib are covalent, irreversible EGFR inhibitors that also inhibit HER2 and ERB4 of the ERB family (Li D, et al. BIBW2992, an irreversible EGFR/HER2 inhibitor highly effective in preclinical lung cancer models. Oncogene 2008; 27: 4702-11; Ou S H, Soo R A. Dacomitinib in lung cancer: a “lost generation” EGFR tyrosine-kinase inhibitor from a bygone era?Drug Des Devel Ther 2015; 9:5641-53). Although afatinib and dacomitinib are more potent EGFR inhibitors approved as first-line therapy for advanced EGFR mutation-positive (Del19 or L858R) NSCLC with longer progression free survival time (PFS) in comparison with gefitinib and erlotinib, EGFR T790M has been developed with time of treatment with afatinib (Tanaka K, et al. Acquisition of the T790M resistance mutation during afatinib treatment in EGFR tyrosine kinase inhibitor-naive patients with non-small cell lung cancer harboring EGFR mutations. Onco-target 2017; 8:68123-30). EGFR T790M confers resistance to dacomitinib in vitro studies (Kobayashi Y, et al. EGFR T790M and C797S mutations as mechanisms of acquired resistance to dacomitinib. J Thorac Oncol 2018; 13: 727-31).

The third-generation EGFR inhibitor Osimertinib is also an irreversible inhibitor targeting both EGFR activating mutations (Del19 and L858R) and T790M resistant double mutations, with selectivity over the wild-type EGFR (Finlay M R, et al. Discovery of a potent and selective EGFR inhibitor (AZD9291) of both sensitizing and T790M resistance mutations that spares the wild type form of the receptor. J Med Chem 2014; 57:8249-67). Osimertinib was first approved for patients with metastatic EGFR T790M mutation-positive NSCLC after failure of first-line EGFR inhibitors, and later approved in the first-line setting for patients with EGFR mutation-positive NSCLC following the phase III FLAURA trial with head-to-head trials comparing with erlotinib or gefitinib (Soria J C, et al. Osimertinib in untreated EGFR-mutated advanced non-small-cell lung cancer. N Engl J Med 2018; 378:113-25). The mutation C797S at the EGFR covalent binding residue with irreversible EGFR inhibitor Osimertinib has been detected in Osimertinib-resistant patients (Ramalingam S S, et al. Mechanisms of acquired resistance to first-line osimertinib: preliminary data from the phase III FLAURA study. Presented at the ESMO 2018).

Genetic alterations of the rearranged during transfection (RET) gene occur in diverse cancers including non-small-cell lung cancer and thyroid carcinoma (Drilon A, et al. Targeting RET-driven cancers: lessons from evolving preclinical and clinical landscapes. Nat Rev Clin Oncol. 2018, 15:151-167). Multikinase inhibitors lenvatinib, sorafenib and cabozantinib were approved for certain thyroid cancers. Recently the highly selective RET inhibitors selpercatinib and pralsetinib were approved for treating metastatic RET fusion-positive non-small-cell lung cancer (NSCLC), advanced/metastatic RET-altered medullary thyroid cancer (MTC) and papillary thyroid carcinoma (PTC). Acquired resistance RET mutations following treatment with multikinase inhibitors or selective RET inhibitors either from RET-mutated patients or cell lines have been reported including gatekeeper mutations V804M and V804L, hinge mutations Y806N and Y806C, solvent front mutations G810A, G810C, G810S, G810V and G810R, and other RET kinase domain mutations, e.g. V738A and S904F (Subbiah V, et al. Structural basis of acquired resistance to selpercatinib and pralsetinib mediated by non-gatekeeper RET mutations. Ann Oncol. 2020 Nov. 5:S0923-7534(20)43127-8). With sequential treatments of multiple RET inhibitors, acquired compound mutations, e.g. RET M918T/V804M, M918T/V804M/G810C, V804M/G810C or other combinations likely cause refractory to current multikinase and selective RET inhibitors in clinic. Therefore, it is necessary to develop new generation RET inhibitors that can target both primary and secondary RET mutations for RET-mutated patients with or without treatment of approved RET inhibitors.

Chronic myeloid leukemia (CML) is characterized by the Philadelphia (Ph) chromosome, which results from t(9;22)(q34;q11) balanced reciprocal translocation leading to the generation of the BCR-ABL oncogene that encodes for the chimeric BCR-ABL1 oncoprotein. (Salesse S, Verfaillie C M. BCR/ABL: from molecular mechanisms of leukemia induction to treatment of chronic myelogenous leukemia. Oncogene. 2002, 21(56):8547-59). Imatinib, a selective BCR-ABL1 kinase inhibitor was the first approved tyrosine kinase inhibitor that have revolutionized the treatment and outcomes for patients with CML. However, mutations in the BCR-ABL1 kinase domain render resistance to imatinib treatment. More than 50 mutation sites and more than 70 individual mutations conferring different levels of resistance have been found in CML patients (Apperley J: Part I: Mechanisms of resistance to imatinib in chronic myeloid leukaemia. Lancet Oncol 2007, 8:1018-1029). Although the more potent, second generation BCR-ABL1 inhibitors have been approved, none of them are potent against all of imatinib-resistance mutations. Y253H, E255V, F359V and Q252H confer intermediate resistance to nilotinib, and E255V, F317L, Q252H to dasatinib while T315I is resistant to nilotinib, dasatinib and bosutinib (O'Hare T, et al. Bcr-Abl kinase domain mutations, drug resistance, and the road to a cure for chronic myeloid leukemia. Blood, 2007, 110, 2242-2249). The third generation BCR-ABL1 inhibitor ponatinib is potent against T315I, however, not potent against T315L and T315M. A wide variety of compound mutations after sequential treatment with multiple BCR-ABL1 inhibitors bring a new challenge for current approved BCR-ABL1 inhibitors (Zabriskie M S, et al. Extreme mutational selectivity of axitinib limits its potential use as a targeted therapeutic for BCR-ABL1-positive leukemia. Leukemia 2016, 30(6):1418-21). In addition, none of the currently available BCR-ABL1 inhibitors is absolutely safe and the widespread prescription of 2nd or 3rd generation BCR-ABL1 inhibitors is tempered by their toxicity. Therefore, it is necessary to develop new generation BCR-ABL1 inhibitors that can target both BCR-ABL1 fusion protein and acquired mutations with better safety profile.

FMS-like tyrosine kinase 3 (FLT3) is a receptor tyrosine kinase that is normally expressed by hematopoietic stem or progenitor cells and plays an important role in the early stages of both myeloid and lymphoid lineage development. Mutations of FLT3 are found in approximately 30% of newly diagnosed AML cases and occur as either internal tandem duplication (ITD) (≈25%) or point mutations in the tyrosine kinase domain (TKD) (7-10%) (Daver N, et al. Targeting FLT3 mutations in AML: review of current knowledge and evidence. Leukemia 2019, 33(2):299-312). Both FLT3-ITD and FLT3-TKD mutations constitutively activate FLT3 kinase activity, resulting in proliferation and survival of AML. The multikinase inhibitor midostaurin was approved for the frontline treatment of patients with FLT3-mutated (either ITD or TKD) AML in combination with induction chemotherapy and the second-generation selective FLT3 inhibitor gilteritinib as a single agent for patients with relapsed or refractory FLT3-mutated AML. Although encouraging results with FLT3 inhibitor-based treatments, many patients still fail to respond to FLT3 inhibitor therapy or subsequently relapse. One of the resistance mechanisms is the development of secondary mutations in the FLT3 kinase domain including the mutations at the activating residues (e.g. D835, 1836, D839, Y842) or gatekeeper residue (e.g. F691) (Short N J, et al Advances in the Treatment of Acute Myeloid Leukemia: New Drugs and New Challenges. Cancer Discov. 2020 April; 10(4):506-525). Therefore, it is necessary to develop new generation FLT3 inhibitors that can target both primary and secondary FLT3 mutations for FLT3-mutated cancer patients with or without treatment of approved FLT3 inhibitors.

Gastrointestinal stromal tumour (GIST) is a mesenchymal tumour of the gastrointestinal tract and accounts for 18% of all human sarcomas (Corless C L, et al Gastrointestinal stromal tumours: Origin and molecular oncology. Nat Rev Cancer. 2011, 11:865-878). The gain-of-function mutations of KIT or PDGFRA receptor tyrosine kinase have been characterized as oncogenic driver mutations in approximately 80-90% of GISTs (O'Brien K M, et al. Gastrointestinal stromal tumors, somatic mutations and candidate genetic risk variants. PLoS One. 8:e621192013). The KIT and PDGFRA inhibitor imatinib has been approved as first-line therapy for GIST patients with unresectable, recurrent, or metastatic disease, except those with PDGFRA D842V mutations. Most patients with initial clinical benefit from imatinib eventually progress after 20-24-month treatment (Blanke, C. D. et al. Long-term results from a randomized phase II trial of standard-versus higher-dose imatinib mesylate for patients with unresectable or metastatic gastrointestinal stromal tumors expressing KIT. J. Clin. Oncol. 2008, 26, 620-625). Oncogenically-activated KIT continues to be the key driver of GIST proliferation and survival after imatinib failure in up to 90% of the patients, due to reactivation of KIT signalling by tumour subclones with heterogeneous secondary KIT mutations (Serrano C, et al. Complementary activity of tyrosine kinase inhibitors against secondary kit mutations in imatinib-resistant gastrointestinal stromal tumours. British Journal of Cancer, 2019, 120: 612-620). Sunitinib and regorafenib showed inhibitory activities only against certain secondary mutations, leading to limited efficacies as second and third line therapies, respectively. Therefore, it is necessary to develop new generation KIT and/or PDGFRA inhibitors that can target both primary and full spectrum of secondary mutations for GIST patients with or without treatment of approved KIT and/or PDGFR inhibitors.

Overall, it is urgent to develop next generation kinase inhibitors that can target both primary mutations and clinical emerging secondary mutations for achieving better efficacy and longer treatment duration as first-line therapy or overcoming resistance mutations for refractory patients. For example, it is necessary to develop a new generation reversible EGFR inhibitors that are potent against oncogenic driver EGFR mutations, such as L858R, Del19, L858R/T790M, Del19/T790M, L858R/C979S, and Del19/C979S, as well as other emrging and established resistance mutations, while maintaining good selectivity over wild-type EGFR.

### SUMMARY

In one aspect, the disclosure relates to a compound of the formula I, or a pharmaceutically acceptable salt thereof,

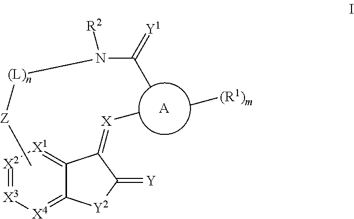

wherein
A is a 5- to 10-membered heteroarylene or C₆-C₁₀ arylene;
each L is independently —C(R³)(R⁴)—, —C(O)—, —O—, —N(R⁵)—, —S—, —S(O)— or —S(O)₂—, provided that (L)ₙ does not comprise a —O—O—, a —O—S—, or a —O—N(R⁵)— bond;
X is N or C(R⁶)
X¹ is N or C(R⁷);
X² is N or C(R⁸);
X³ is N or C(R⁹)
X⁴ is N or C(R¹⁰);
Y and Y¹ are each independently O or S;
Y² is —O—, —N(R¹¹)—, or —S—;
Z is a 3- to 7-membered heterocycloalkylene, C₃-C₆ cycloalkylene, C₆-C₁₀ arylene, 5- to 10-membered heteroarylene, —C(R¹²)(R¹³)—, —C(O)—, —O—, —N(R¹⁴)—, —S—, —S(O)— or —S(O)₂—, wherein each hydrogen atom in 3- to 7-membered heterocycloalkylene, C₃-C₆ cycloalkylene, C₆-C₁₀ arylene, and 5- to 10-membered heteroarylene is independently optionally substituted by deuterium, halogen, C₁-C₆ alkyl, C₁-C₆ haloalkyl, C₂-C₆ alkenyl, C₂-C₆ alkynyl, C₃-C₆ cycloalkyl, 3- to 7-membered heterocycloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
Z¹ is —NR²C(Y¹)—, —C(Y¹)NR²—, —O—, —N(R²)—, —S—, —S(O)— or —S(O)₂—;
each R¹ is independently deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R², R⁵, R¹¹, or R¹⁴ is independently H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}′, —CN, or —NO₂;
each R³, R⁴, R¹² and R¹³ is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{e}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)ORC, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, —NO₂, or two of R³, R⁴, R¹², and R¹³ taken together with the carbon or carbons to which they are attached form a C₃-C₆ cycloalkyl or a 4- to 6-membered heterocycloalkyl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, or 4- to 6-membered heterocycloalkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R⁶ is H, deuterium, halogen, C₁-C₆ alkyl, C₂-C₆ alkenyl, C₂-C₆ alkynyl, or —CN;
each of R⁷ and R⁸ is independently a bond to Z, H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂; wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; provided that one of R⁷ or R⁸ is a bond to Z;
each of R⁹ and R¹⁰ is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂; or R⁸ and R⁹ or R⁹ and R¹⁰ taken together with the carbons to which they are attached form a C₄-C₆ cycloalkyl, a 4- to 7-membered heterocycloalkyl, or a C₆-C₁₀ aryl, wherein each hydrogen atom in C₁-C₆ alkyl, C₂-C₆ alkenyl, C₂-C₆ alkynyl, C₃-C₆ cycloalkyl, C₄-C₆ cycloalkyl, 3- to 7-membered heterocycloalkyl, C₆-C₁₀ aryl, 5- to 10-membered heteroaryl, or 4- to 7-membered heterocycloalkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each R^{a}, R^{b}, R^{e}, R^{d}, R^{e}, and R^{f} is independently selected from the group consisting of H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkyl-C₆-C₁₀ aryl, and 5- to 10-membered heteroaryl;
m is 0, 1, 2, 3, or 4; and
n is 2, 3, 4, 5, 6, 7, or 8.

In some embodiments, the disclosure provides a compound of the formula II, or a pharmaceutically acceptable salt thereof,

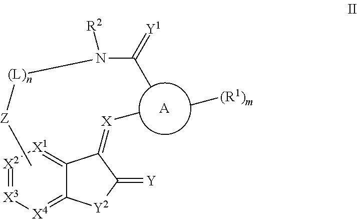

wherein R¹, R², A, L, X, X¹, X², X³, X⁴, Y, Y¹, Y², Z, m and n are as described herein.

In some embodiments, the disclosure provides a compound of the formula III, or a pharmaceutically acceptable salt thereof,

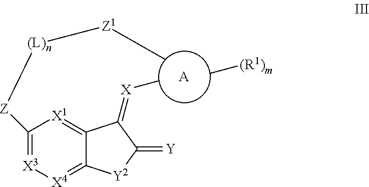

wherein R¹, A, L, X, X¹, X², X³, X⁴, Y, Y², Z, Z¹, m and n are as described herein.

In some embodiments, the disclosure provides a compound of the formula IV, or a pharmaceutically acceptable salt thereof,

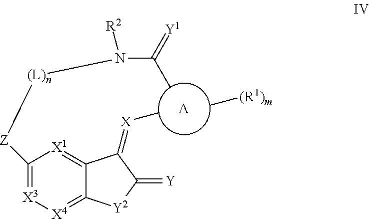

wherein R¹, R², A, L, X, X¹, X², X³, X⁴, Y, Y¹, Y², Z, m and n are as described herein.

In some embodiments, the disclosure provides a compound of the formula V, or a pharmaceutically acceptable salt thereof,

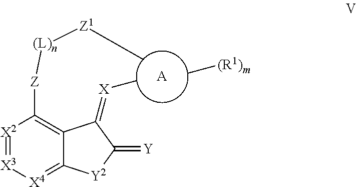

wherein R¹, A, L, X, X¹, X², X³, X⁴, Y, Y¹, Z, Z¹, m and n are as described herein.

In some embodiments, the disclosure provides a compound of the formula VI, or a pharmaceutically acceptable salt thereof,

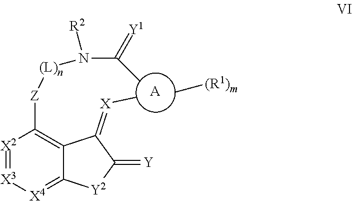

wherein R¹, R², A, L, X, X¹, X², X³, X⁴, Y, Y¹, Y², Z, m and n are as described herein.

In some embodiments, the disclosure provides a compound of the formula VII, or a pharmaceutically acceptable salt thereof,

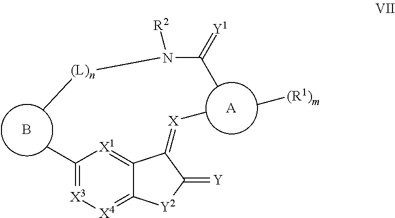

wherein R¹, R², A, B, L, X, X¹, X², X³, X⁴, Y, Y¹, Y², Z, m and n are as described herein.

In some embodiments, the disclosure provides a compound of the formula VIII, or a pharmaceutically acceptable salt thereof,

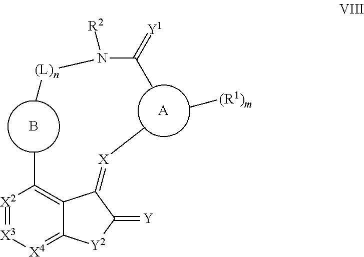

wherein R¹, R², A, B, L, X, X¹, X², X³, X⁴, Y, Y¹, Y², Z, m and n are as described herein.

In some aspects of each of the above embodiments, Ring B (Z) is not

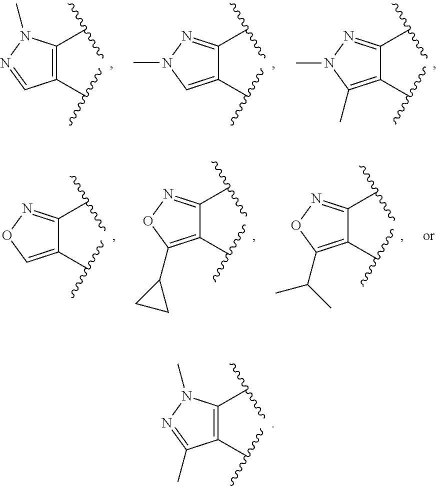

In some embodiments, Ring B (Z) is not

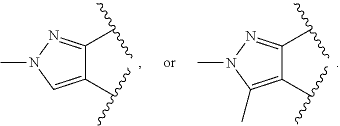

In some aspects of the embodiments herein, C(R⁹) is H. In some aspects of the embodiments herein, C(R⁹) is not —Cl. In some embodiments, C(R¹⁰) is H. In some aspects of the embodiments herein, C(R¹⁰) is not —Cl.

In some aspects of the embodiments herein, the compound is not a compound wherein Ring B (Z) is

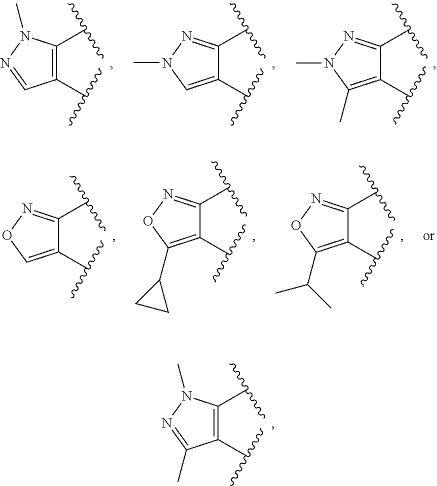

and R⁹ and/or R¹⁰ is not H. In some embodiments, the compound is not a compound wherein Ring B (Z) is

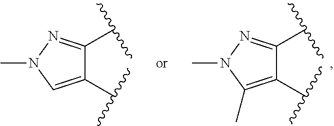

and R⁹ and/or R⁰ is not H. In some aspects of the embodiments herein, the compound is not a compound wherein X¹ is C(R⁷), X³ is C(R⁹), X⁴ is C(R¹⁰), R⁹ and/or R¹⁰ is not H, and Ring B (Z) is

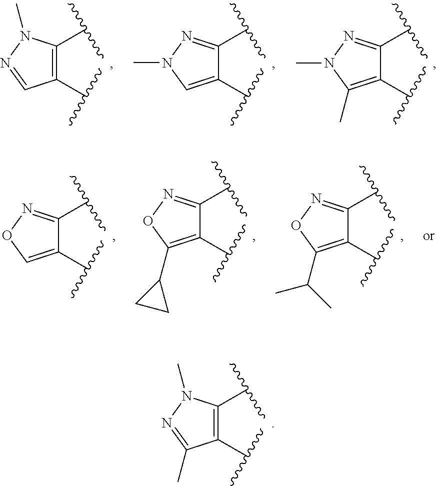

In some aspects of the embodiments herein, the compound is not a compound wherein X¹ is C(R⁷), X³ is C(R⁹), X⁴ is C(R¹⁰), R⁹ and/or R¹⁰ is not H, and Ring B (Z) is

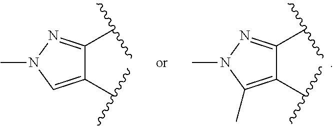

In some aspects of the embodiments herein, the compound is not a compound wherein X¹ is C(R⁷), X³ is C(R⁹), X⁴ is C(R¹⁰), R⁹ and/or R¹ is —Cl, and Ring B (Z) is

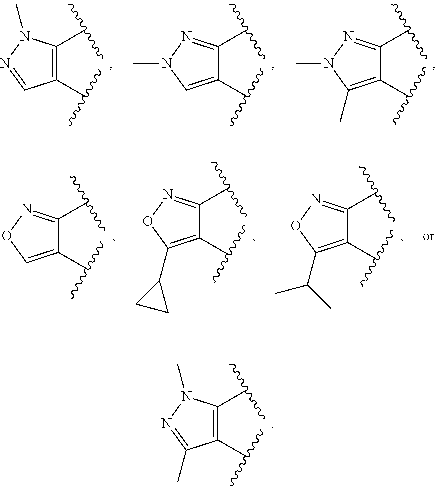

In some aspects of the embodiments herein, the compound is not a compound wherein X¹ is C(R⁷), X³ is C(R⁹), X⁴ is C(R¹⁰), R⁹ and/or R¹⁰ is —Cl, and Ring B (Z) is

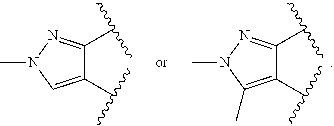

In some aspects of the embodiments herein, X¹ is C(R⁷), X³ is C(R⁹), X⁴ is C(R¹⁰), and R⁹ and/or R⁰ is not —Cl. In some embodiments, X¹ is C(R⁷), X³ is C(R⁹), X⁴ is C(R¹⁰), R⁹ and/or R¹⁰ is not —Cl, and Ring B (Z) is not

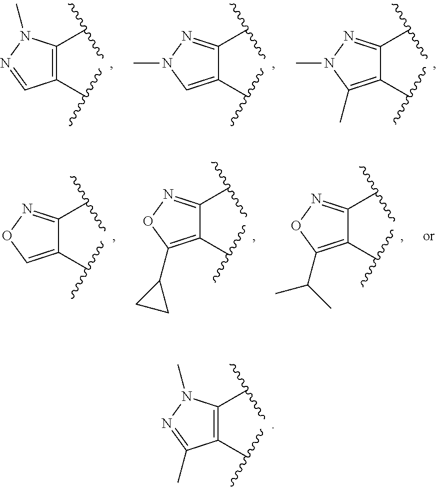

In some aspects of the embodiments herein, X¹ is C(R⁷), X³ is C(R⁹), X⁴ is C(R¹⁰), R⁹ and/or R¹⁰ is not —Cl, and Ring B (Z) is not

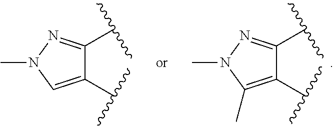

In certain embodiments of the above aspects, the compound of Formula (I)-(VIII) is a compound selected from those species described or exemplified in the detailed description below.

In further aspects, the disclosure relates to a pharmaceutical composition comprising at least one compound of Formula (I)-(VIII) or a pharmaceutically acceptable salt thereof.

Pharmaceutical compositions according to the disclosure may further comprise a pharmaceutically acceptable excipient.

In further aspects, the disclosure relates to a compound of Formula (I)-(VIII), or a pharmaceutically acceptable salt thereof, for use as a medicament.

In further aspects, the disclosure relates to a method of treating disease, such as cancer comprising administering to a subject in need of such treatment an effective amount of at least one compound of Formula (I)-(VIII), or a pharmaceutically acceptable salt thereof.

In further aspects, the disclosure relates to use of a compound of Formula (I)-(VIII), or a pharmaceutically acceptable salt thereof, in the preparation of a medicament for the treatment of disease, such as cancer, and the use of such compounds and salts for treatment of such diseases.

In further aspects, the disclosure relates to a method of inhibiting a tyrosine kinase, such as EGFR, comprising contacting a cell comprising one or more of kinase with an effective amount of at least one compound of Formula (I)-(VIII), or a pharmaceutically acceptable salt thereof, and/or with at least one pharmaceutical composition of the disclosure, wherein the contacting is in vitro, ex vivo, or in vivo.

Additional embodiments, features, and advantages of the disclosure will be apparent from the following detailed description and through practice of the disclosure. The compounds of the present disclosure can be described as embodiments in any of the following enumerated clauses. It will be understood that any of the embodiments described herein can be used in connection with any other embodiments described herein to the extent that the embodiments do not contradict one another.

1. A compound of the formula I, or a pharmaceutically acceptable salt thereof,

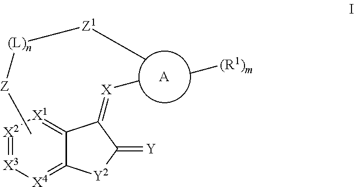

wherein
A is a 5- to 10-membered heteroarylene or C₆-C₁₀ arylene;
each L is independently —C(R³)(R⁴)—, —C(O)—, —O—, —N(R⁵)—, —S—, —S(O)— or —S(O)₂—, provided that (L)ₙ does not comprise a —O—O—, a —O—S—, or a —O—N(R⁵)— bond;
X is N or C(R⁶)
X¹ is N or C(R⁷);
X² is N or C(R⁸);
X³ is N or C(R⁹)
X⁴ is N or C(R¹⁰);
Y and Y¹ are each independently O or S;
Y² is —O—, —N(R¹¹)—, or —S—;
Z is a 3- to 7-membered heterocycloalkylene, C₃-C₆ cycloalkylene, C₆-C₁₀ arylene, 5- to 10-membered heteroarylene, —C(R¹²)(R¹³)—, —C(O)—, —O—, —N(R¹⁴)—, —S—, —S(O)— or —S(O)₂—, wherein each hydrogen atom in 3- to 7-membered heterocycloalkylene, C₃-C₆ cycloalkylene, C₆-C₁₀ arylene, and 5- to 10-membered heteroarylene is independently optionally substituted by deuterium, halogen, C₁-C₆ alkyl, C₁-C₆ haloalkyl, C₂-C₆ alkenyl, C₂-C₆ alkynyl, C₃-C₆ cycloalkyl, 3- to 7-membered heterocycloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
Z¹ is —NR²C(Y¹)—, —C(Y¹)NR²—, —O—, —N(R²)—, —S—, —S(O)— or —S(O)₂—,
each R¹ is independently deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R², R⁵, R¹¹, or R¹⁴ is independently H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}′, —CN, or —NO₂;
each R³, R⁴, R¹² and R¹³ is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{e}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)ORC, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, —NO₂, or two of R³, R⁴, R¹², and R¹³ taken together with the carbon or carbons to which they are attached form a C₃-C₆ cycloalkyl or a 4- to 6-membered heterocycloalkyl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, or 4- to 6-membered heterocycloalkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R⁶ is H, deuterium, halogen, C₁-C₆ alkyl, C₂-C₆ alkenyl, C₂-C₆ alkynyl, or —CN;
each of R⁷ and R⁸ is independently a bond to Z, H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂; wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; provided that one of R⁷ or R⁸ is a bond to Z;
each of R⁹ and R¹⁰ is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂; or R⁸ and R⁹ or R⁹ and R¹⁰ taken together with the carbons to which they are attached form a C₄-C₆ cycloalkyl, a 4- to 7-membered heterocycloalkyl, or a C₆-C₁₀ aryl, wherein each hydrogen atom in C₁-C₆ alkyl, C₂-C₆ alkenyl, C₂-C₆ alkynyl, C₃-C₆ cycloalkyl, C₄-C₆ cycloalkyl, 3- to 7-membered heterocycloalkyl, C₆-C₁₀ aryl, 5- to 10-membered heteroaryl, or 4- to 7-membered heterocycloalkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each R^{a}, R^{b}, R^{e}, R^{d}, R^{e}, and R^{f} is independently selected from the group consisting of H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkyl-C₆-C₁₀ aryl, and 5- to 10-membered heteroaryl;
m is 0, 1, 2, 3, or 4; and
n is 2, 3, 4, 5, 6, 7, or 8.

2. The compound of clause 1 having the formula IV

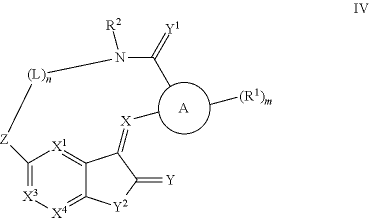

or a pharmaceutically acceptable salt thereof.

3. The compound of clause 1 having the formula VI

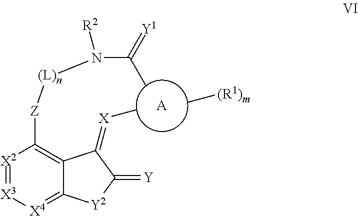

or a pharmaceutically acceptable salt thereof.

4. The compound of any one of clauses 1 to 3, or a pharmaceutically acceptable salt thereof, wherein A is phenylene, furanylene, thiophenylene, pyrrolylene, oxazolylene, isoxazolylene, thiazolylene, isothiazolylene, pyrazolylene, imidazolylene, oxadiazolylene, thiadiazolylene, triazolylene, pyridinylene, pyrazinylene, pyrimidinylene, pyridazinylene, or triazinylene.

5. The compound of any one of the preceding clauses, or a pharmaceutically acceptable salt thereof, wherein A is a pyrrolylene.

6. The compound of any one of the preceding clauses, or a pharmaceutically acceptable salt thereof, wherein A is

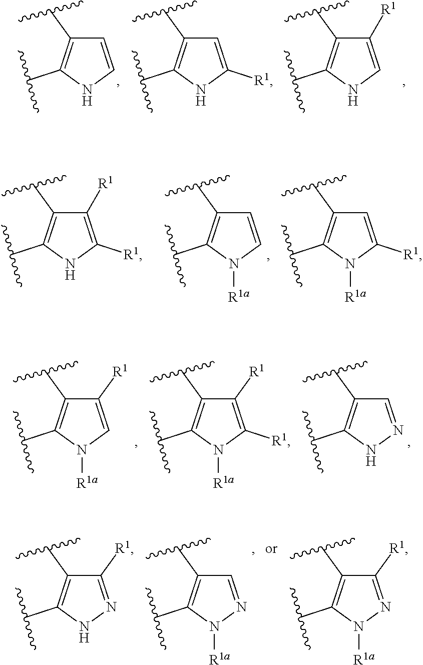

wherein R^{1a} is C₁-C₆ alkyl, —C(O)R^{a}, —C(O)OR^{a}, —C(O)NR^{a}R^{b}, or —P(O)₂OR^{a}, wherein each hydrogen atom in C₁-C₆ alkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

7. The compound of any one of the preceding clauses, or a pharmaceutically acceptable salt thereof, wherein A is

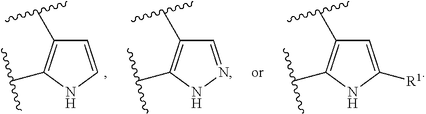

8. The compound of any one of the preceding clauses, or a pharmaceutically acceptable salt thereof, each R¹ is —CN or C₁-C₆ alkyl, wherein each hydrogen atom in C₁-C₆ alkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{c}, —P(O)₂OR^{e}, —CN, or —NO₂.

9. The compound of any one of the preceding clauses, or a pharmaceutically acceptable salt thereof, each R¹ is —CN or methyl.

10. The compound of any one of the preceding clauses, or a pharmaceutically acceptable salt thereof, R^{1a} is methyl.

11. The compound of any one of the preceding clauses, or a pharmaceutically acceptable salt thereof, wherein R² is H or C₁-C₆ alkyl, wherein each hydrogen atom in C₁-C₆ alkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}—CN, or —NO₂.

12. The compound of any one of the preceding clauses, or a pharmaceutically acceptable salt thereof, wherein R² is H or methyl.

13. The compound of any one of the preceding clauses, or a pharmaceutically acceptable salt thereof, wherein Z is a 5- or 6-membered heteroarylene, wherein each hydrogen atom in 5- or 6-membered heteroarylene is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

14. The compound of any one of the preceding clauses, or a pharmaceutically acceptable salt thereof, wherein Z is pyrazolylene, oxazolylene, thiazolylene, pyridinylene, pyrimidinylene, or pyridin-2-onylene, wherein each hydrogen atom in pyrazolylene, oxazolylene, thiazolylene, pyridinylene, pyrimidinylene, and pyridin-2-onylene is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

15. The compound of any one of the preceding clauses, or a pharmaceutically acceptable salt thereof, wherein Z is

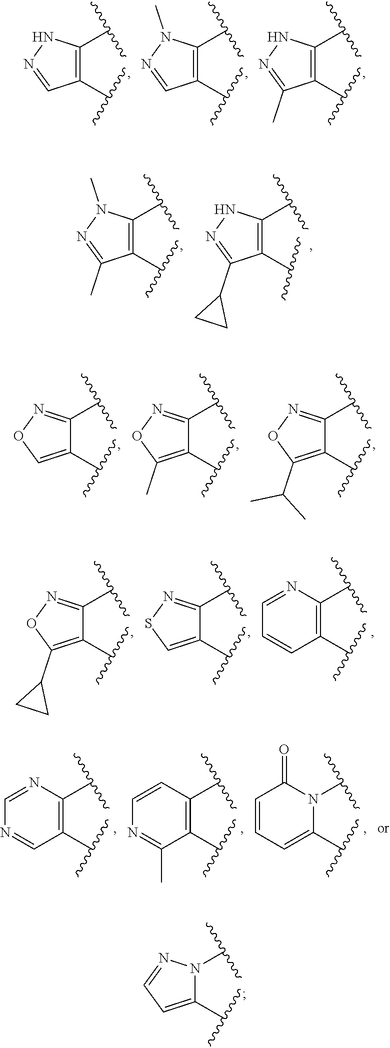

### or Z is not

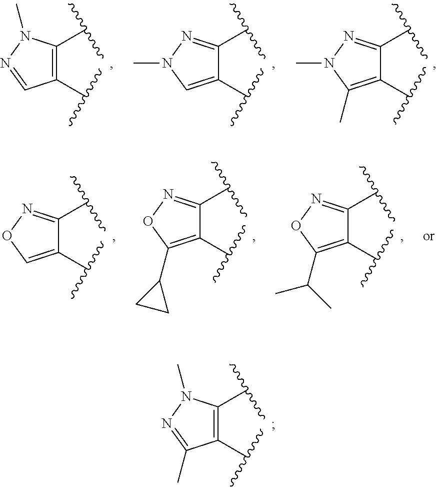

### or Z is not

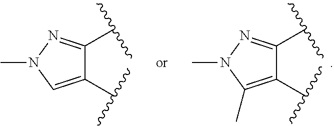

16. The compound of any one of clauses 1 to 12, or a pharmaceutically acceptable salt thereof, wherein Z is C₆-C₁₀ arylene, wherein each hydrogen atom in C₆-C₁₀ arylene is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

17. The compound of any one of clauses 1 to 12 or 16, or a pharmaceutically acceptable salt thereof, wherein Z is phenylene, wherein each hydrogen atom in phenylene is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

18. The compound of any one of clauses 1 to 12, 16, or 17, or a pharmaceutically acceptable salt thereof, wherein Z is

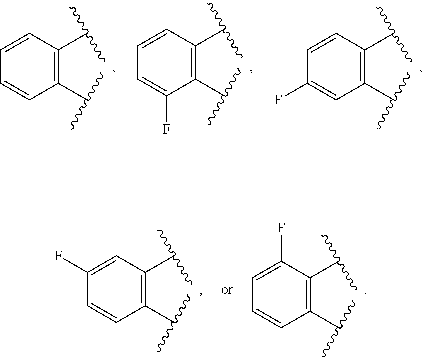 [SMILES: *c1ccccc1* ; *c1cccc(F)c1* ; *c1ccc(F)cc1* ; *c1ccc(F)cc1* ; *c1cccc(F)c1*]

19. The compound of any one of clauses 1 to 12, or a pharmaceutically acceptable salt thereof, wherein Z is 3- to 7-membered heterocycloalkylene, wherein each hydrogen atom in 3- to 7-membered heterocycloalkylene is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

20. The compound of any one of clauses 1 to 12 or 19, or a pharmaceutically acceptable salt thereof, wherein Z is pyrrolidonylene or azetidinylene, wherein each hydrogen atom in pyrrolidonylene and azetidinylene is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

21. The compound of any one of clauses 1 to 12, or a pharmaceutically acceptable salt thereof, wherein Z is —C(R¹²)(R¹³)—, —O—, —N(R¹⁴)—, —S—, —S(O)— or —S(O)₂—.

22. The compound of any one of clauses 1 to 12 or 21, or a pharmaceutically acceptable salt thereof, wherein Z is —C(R¹²)(R¹³)—.

23. The compound of any one of the preceding clauses, wherein R¹² and R¹³ are independently selected from the group consisting of H, deuterium, fluoro, chloro, bromo, —OR^{e}, and C₁-C₆ alkyl; or R¹² and R¹³ taken together with the carbon to which they are attached form a C₃-C₆ cycloalkyl or a 4- to 6-membered heterocycloalkyl, wherein each hydrogen atom in C₃-C₆ cycloalkyl or 4- to 6-membered heterocycloalkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

24. The compound of any one of the preceding clauses, wherein R¹² is H and R¹³ is methyl.

25. The compound of any one of the preceding clauses, wherein R¹² is methyl and R¹³ is H.

26. The compound of any one of the preceding clauses, wherein R¹² and R¹³ are H.

27. The compound of any one of the preceding clauses, wherein R¹² is methyl and R¹³ is —OH.

28. The compound of any one of the preceding clauses, wherein R¹² is —OH and R¹³ is methyl.

29. The compound of any one of clauses 1 to 12 or 21, or a pharmaceutically acceptable salt thereof, wherein Z is —O—.

30. The compound of any one of clauses 1 to 12 or 21, or a pharmaceutically acceptable salt thereof, wherein Z is —N(R¹⁴)—.

31. The compound of any one of the preceding clauses, or a pharmaceutically acceptable salt thereof, wherein R¹⁴ is H, deuterium, C₁-C₆ alkyl, or C₃-C₆ cycloalkyl.

32. The compound of any one of the preceding clauses, or a pharmaceutically acceptable salt thereof, wherein R¹⁴ is H, methyl, or cyclopropyl.

33. The compound of any one of clauses 1 to 12 or 21, or a pharmaceutically acceptable salt thereof, wherein Z is —S—.

34. The compound of any one of clauses 1 to 12 or 21, or a pharmaceutically acceptable salt thereof, wherein Z is —S(O)₂—.

35. The compound of any one of the preceding clauses, or a pharmaceutically acceptable salt thereof, wherein n is 3.

36. The compound of any one of the preceding clauses, or a pharmaceutically acceptable salt thereof, wherein n is 4.

37. The compound of any one of the preceding clauses, or a pharmaceutically acceptable salt thereof, wherein n is 5.

38. The compound of any one of the preceding clauses, or a pharmaceutically acceptable salt thereof, wherein n is 6.

39. The compound of any one of the preceding clauses, or a pharmaceutically acceptable salt thereof, wherein n is 7.

40. The compound of any one of the preceding clauses, or a pharmaceutically acceptable salt thereof, wherein each L is independently selected from the group consisting of —C(O)—, —O—, —CH₂—, —C(H)(CH₃)—, —C(H)(OH)—, —C(H)(C(O)OR^{c})—, —C(H)(C(O)NR^{c}R^{d})—, —NH—, and —NCH₃—.

41. The compound of any one of the preceding clauses, or a pharmaceutically acceptable salt thereof, wherein X is C(R⁶).

42. The compound of any one of the preceding clauses, or a pharmaceutically acceptable salt thereof, wherein R⁶ is H.

43. The compound of any one of the preceding clauses, or a pharmaceutically acceptable salt thereof, wherein Y is O.

44. The compound of any one of the preceding clauses, or a pharmaceutically acceptable salt thereof, wherein Y¹ is O.

45. The compound of any one of the preceding clauses, or a pharmaceutically acceptable salt thereof, wherein Y² is —N(R¹¹)—.

46. The compound of any one of the preceding clauses, or a pharmaceutically acceptable salt thereof, wherein X¹, when present, and X³ are N.

47. The compound of any one of clauses 1 to 45, or a pharmaceutically acceptable salt thereof, wherein X¹, when present, and X⁴ are N.

48. The compound of any one of clauses 1 to 45, or a pharmaceutically acceptable salt thereof, wherein X³ and X⁴ are N.

49. The compound of any one of clauses 1 to 45, or a pharmaceutically acceptable salt thereof, wherein X¹, when present, is N.

50. The compound of any one of clauses 1 to 45, or a pharmaceutically acceptable salt thereof, wherein X², when present, is N.

51. The compound of any one of clauses 1 to 45, or a pharmaceutically acceptable salt thereof, wherein X³ is N.

52. The compound of any one of clauses 1 to 45, or a pharmaceutically acceptable salt thereof, wherein X⁴ is N.

53. The compound of any one of clauses 1 to 45, or a pharmaceutically acceptable salt thereof, wherein X¹ is C(R⁷), X³ is C(R⁹), and X⁴ is C(R¹⁰).

54. The compound of any one of the preceding clauses, or a pharmaceutically acceptable salt thereof, wherein C(R⁷), when present, is independently H, deuterium, fluoro, chloro, —CN, or methyl.

55. The compound of any one of the preceding clauses, or a pharmaceutically acceptable salt thereof, wherein C(R⁸), when present, is independently H, deuterium, fluoro, chloro, —CN, or methyl.

56. The compound of any one of the preceding clauses, or a pharmaceutically acceptable salt thereof, wherein C(R⁹), when present, is independently H, deuterium, fluoro, chloro, —CN, or methyl.

57. The compound of any one of the preceding clauses, or a pharmaceutically acceptable salt thereof, wherein C(R¹⁰), when present, is H

58. The compound of any one of the preceding clauses, or a pharmaceutically acceptable salt thereof, wherein -(L)ₙ- is —(CH₂)₂—, —(CH₂)₃—, —(CH₂)₄—, —(CH₂)₅—, —(CH₂)₆—, —C(O)NH—(CH₂)₂O(CH₂)₂—, —C(O)N(CH₃)—(CH₂)₂O(CH₂)₂—, —NHC(O)CH₂O(CH₂)₂—, —N(CH₃)—C(O)CH₂O(CH₂)₂—, —CH₂O(CH₂)₂—, —(CH₂)₂O(CH₂)₂—, —(CH₂)₂S(CH₂)₂—, —O(CH₂)₂S(CH₂)₂—, —(CH₂)₂SO₂(CH₂)₂—, —O(CH₂)₂SO₂(CH₂)₂—, —(CH₂)₂SO(CH₂)₂—, —O(CH₂)₂SO(CH₂)₂—, —(CH₂)₂O(C(H)(C(O)N(H) (azetidin-3-yl))-CH₂—, —(CH₂)₂O(C(H)(C(O)N(H)(CH₃))—CH₂—, —(CH₂)₂O(C(H)(C(O)N(CH₃)₂)—CH₂—, —(CH₂)₂O(C(H)(C(O)N(H)(piperidin-4-yl))-CH₂—, —(CH₂)₂O(C(H)(C(O)N(H)(pyrrolidin-3-yl))-CH₂—, —(CH₂)₂O(C(H)(C(O)N(H)(4-methylpiperazin-1-yl))-CH₂—, —(CH₂)₂O(C(H)(C(O)OCH₃)—CH₂—, —(CH₂)₃O(CH₂)₂—, —(CH₂)₂O(CH₂)₃—, —CH₂CH(CH₃)—O(CH₂)₂—, —CH(CH₃)—CH₂O(CH₂)₂—, —O(CH₂)₂—, —O—(CH₂)₃—, —OCH₂O(CH₂)₂—, —O—CH₂CH(OH)CH₂—, —O—(CH₂)₂O(CH₂)₂—, —O—CH₂CH(CH₃)—O(CH₂)₂—, —O—CH(CH₃)—CH₂O(CH₂)₂—, —O—(CH₂)₂NH—(CH₂)₂—, —O—CH₂CH(CH₃)—NH—(CH₂)₂—, —O—CH(CH₃)—CH₂NH—(CH₂)₂—, —CH₂NH—(CH₂)₂—, —(CH₂)₂NH—(CH₂)₂—, —CH₂CH(CH₃)—NH—(CH₂)₂—, —CH(CH₃)—CH₂NH—(CH₂)₂—, —O—(CH₂)₂N(CH₃)—(CH₂)₂—, —O—CH₂CH(CH₃)—N(CH₃)—(CH₂)₂—, —O—CH(CH₃)—CH₂N(CH₃)—(CH₂)₂—, —CH₂N(CH₃)—(CH₂)₂—, —CH₂N(CH₂CH₃)—(CH₂)₂—, —CH₂N(CH(CH₃))—(CH₂)₂—, —(CH₂)₂N(CH₃)—(CH₂)₂—, —CH₂CH(CH₃)—N(CH₃)—(CH₂)₂—, or —O—CH(CH₃)—CH₂N(CH₃)—(CH₂)₂—.

59. The compound of clause 1, or a pharmaceutically acceptable salt thereof, selected from the group consisting of [3a(4)Z]-10,11-dihydro-2H,13H-16,1-(azenometheno)pyrazolo[4,3-m]dipyrrolo[3,2 f:3′,4′-i][1,4,11]oxadiazacyclotetradecine-3,8(5H,9H)-dione;
[3a(4)Z]-9,10,11,12-tetrahydro-14H-17,1-(azenometheno)pyrazolo[3,4-b]dipyrrolo[3,4-f:2′,3′-i][1,5,12]oxadiazacyclopentadecine-3,8(2H,5H)-dione;
[3a(4)Z]-9,10,11,12-tetrahydro-14H-1,17-(azenometheno)pyrazolo[3,4-b]dipyrrolo[3,4-f:2′,3′-i][1,5,12]oxadiazacyclopentadecine-3,8(2H,5H)-dione;
[3a(4)Z]-9,10,11,12-tetrahydro-14H-1,17-(diazanediylidene)pyrazolo[4,3-n]dipyrrolo[3,2-g:3′,4′-j][1,5]oxazacyclopentadecine-3,8(2H,5H)-dione;
[3a(4)Z]-9,10,11,12-tetrahydro-14H-1,17-(ethanediylidene)pyrazolo[3,4-b]dipyrrolo[3,4-f:2′,3′-i][1,5,12]oxadiazacyclopentadecine-3,8(2H,5H)-dione;
[3a(4)Z]-9,10,11,12-tetrahydro-14H-17,1-(azenometheno)pyrazolo[4,3-n]dipyrrolo[3,2-g:3′,4′-j][1,5]oxazacyclopentadecine-3,8(2H,5H)-dione;
[3a(4)Z]-9,10,11,12-tetrahydro-14H-1,17-(azenometheno)pyrazolo[4,3-n]dipyrrolo[3,2-g:3′,4′-j][1,5]oxazacyclopentadecine-3,8(2H,5H)-dione;
[3a(4)Z]-9,10,11,12-tetrahydro-14H-1,17-(ethanediylidene)pyrazolo[4,3-n]dipyrrolo[3,2-g:3′,4′-j][1,5]oxazacyclopentadecine-3,8(2H,5H)-dione;
[3a(4)Z,11S]-11-hydroxy-9,10,11,12-tetrahydro-14H-1,17-(ethanediylidene)pyrazolo[3,4-b]dipyrrolo[3,4-f:2′,3′-i][1,5,12]oxadiazacyclopentadecine-3,8(2H,5H)-dione;
[19a(20)Z]-2,5-dimethyl-6,7,9,10-tetrahydro-1H,12H-15,17-(ethanediylidene)pyrazolo[4,3-p]dipyrrolo[3,2-i:3′,4′-l][1,4,7,14]dioxadiazacycloheptadecine-4,19(5H,18H)-dione;
[3a(4)Z]-6-methyl-9,10,11,12-tetrahydro-14H-1,17-(ethanediylidene)pyrazolo[3,4-b]dipyrrolo[3,4-f:2′,3′-i][1,5,12]oxadiazacyclopentadecine-3,8(2H,5H)-dione;
[3a(4)Z]-3,8-dioxo-2,3,5,8,9,10,11,12-octahydro-14H-1,17-(ethanediylidene)pyrazolo[3,4-b]dipyrrolo[3,4-f:2′,3′-i][1,5,12]oxadiazacyclopentadecine-6-carbonitrile;
[3a(4)Z]-6-methyl-9,10,11,12-tetrahydro-14H-1,17-(ethanediylidene)imidazo[4,5-i]pyrazolo[3,4-b]pyrrolo[3,4-f][1,5,12]oxadiazacyclopentadecine-3,8(2H,5H)-dione;
[3a(4)Z]-6,15-dimethyl-9,10,11,12-tetrahydro-15H-1,17-(ethanediylidene)pyrazolo[3,4-b]dipyrrolo[3,4-f:2′,3′-i][1,5,12]oxadiazacyclopentadecine-3,8(2H,5H)-dione;
[3a(4)Z]-6-methyl-9,10,11,12-tetrahydro-1,17-(ethanediylidene)[1,2]oxazolo[3,4-b]dipyrrolo[3,4-f:2′,3′-i][1,5,12]oxadiazacyclopentadecine-3,8(2H,5H)-dione;
[3a(4)Z]-6,16-dimethyl-9,10,11,12-tetrahydro-1,17-(ethanediylidene)[1,2]oxazolo[3,4-b]dipyrrolo[3,4-f:2′,3′-i][1,5,12]oxadiazacyclopentadecine-3,8(2H,5H)-dione;
[3a(4)Z]-6-methyl-9,10,11,12-tetrahydro-1,17-(ethanediylidene)dipyrrolo[3,4-f:2′,3′-i][1,2]thiazolo[3,4-b][1,5,12]oxadiazacyclopentadecine-3,8(2H,5H)-dione;
[3a(4)Z]-6,9-dimethyl-9,10,11,12-tetrahydro-14H-1,17-(ethanediylidene)pyrazolo[3,4-b]dipyrrolo[3,4-f:2′,3′-i][1,5,12]oxadiazacyclopentadecine-3,8(2H,5H)-dione;
[3a(4)Z]-6,9-dimethyl-9,10,11,12-tetrahydro-1,17-(ethanediylidene)[1,2]oxazolo[3,4-b]dipyrrolo[3,4-f:2′,3′-i][1,5,12]oxadiazacyclopentadecine-3,8(2H,5H)-dione;
[3a(4)Z]-6,9,16-trimethyl-9,10,11,12-tetrahydro-14H-1,17-(ethanediylidene)pyrazolo[3,4-b]dipyrrolo[3,4-f:2′,3′-i][1,5,12]oxadiazacyclopentadecine-3,8(2H,5H)-dione;
[3a(4)Z]-6,9,16-trimethyl-9,10,11,12-tetrahydro-1,17-(ethanediylidene)[1,2]oxazolo[3,4-b]dipyrrolo[3,4-f:2′,3′-i][1,5,12]oxadiazacyclopentadecine-3,8(2H,5H)-dione;
[3a(4)Z]-6-methyl-9,10,11,12-tetrahydro-1,17-(ethanediylidene)pyrazolo[5,1-c]dipyrrolo[3,2-j:3′,4′-m][1,4,8]triazacyclotetradecine-3,8(2H,5H)-dione;
[3a(4)Z]-6-methyl-9,10,11,12-tetrahydro-17,1-(azenometheno)pyrazolo[1,5-e]dipyrrolo[3,4-i:2′,3′-l][1,5]diazacyclotetradecine-3,8(2H,5H)-dione;
[19a(20)Z]-2-methyl-6,7,9,10-tetrahydro-1H-15,17-(ethanediylidene)pyrazolo[1,5-d]dipyrrolo[3,4-h:2′,3′-k][1,4,7,14]oxatriazacyclohexadecine-4,19(5H,18H)-dione;
[19a(20)Z]-2-methyl-6,7,9,10-tetrahydro-1H-15,17-(azenometheno)pyrazolo[1,5-d]dipyrrolo[3,4-h:2′,3′-k][1,4,14]oxadiazacyclohexadecine-4,19(5H,18H)-dione;
[19a(20)Z]-2-methyl-6,7,9,10-tetrahydro-1H-15,17-(azenometheno)pyrazolo[1,5-d]dipyrrolo[3,4-h:2′,3′-k][1,4,7,14]oxatriazacyclohexadecine-4,19(5H,18H)-dione;
[19a(20)Z]-2-methyl-6,7,9,10-tetrahydro-1H-15,17-(ethanediylidene)pyrazolo[1,5-d]dipyrrolo[3,4-h:2′,3′-k][1,4,14]oxadiazacyclohexadecine-4,19(5H,18H)-dione;
[10R,19a(20)Z]-2,10-dimethyl-6,7,9,10-tetrahydro-1H-15,17-(ethanediylidene)pyrazolo[1,5-d]dipyrrolo[3,4-h:2′,3′-k][1,4,14]oxadiazacyclohexadecine-4,19(5H,18H)-dione;
[19a(20)Z]-2,5-dimethyl-6,7,9,10-tetrahydro-1H-15,17-(ethanediylidene)pyrazolo[1,5-d]dipyrrolo[3,4-h:2′,3′-k][1,4,14]oxadiazacyclohexadecine-4,19(5H,18H)-dione;
[19a(20)Z]-2,5-dimethyl-6,7,9,10-tetrahydro-1H-15,17-(azenometheno)pyrazolo[1,5-d]dipyrrolo[3,4-h:2′,3′-k][1,4,7,14]oxatriazacyclohexadecine-4,19(5H,18H)-dione;
[19a(20)Z]-2-methyl-5,6,7,8,9,10-hexahydro-15,17-(ethanediylidene)pyrazolo[1,5-g]dipyrrolo[3,4-k:2′,3′-n][1,4,7]triazacyclohexadecine-4,19(1H,18H)-dione;
[19a(20)Z]-2,5-dimethyl-5,6,7,8,9,10-hexahydro-15,17-(ethanediylidene)pyrazolo[1,5-g]dipyrrolo[3,4-k:2′,3′-n][1,4,7]triazacyclohexadecine-4,19(1H,18H)-dione;
[3a(4)Z]-6-methyl-10,11,13,14-tetrahydro-2H-1,17-(ethanediylidene)pyrazolo[4,3-m]dipyrrolo[3,2-f:3′,4′-i][1,4,11]oxadiazacyclopentadecine-3,8(5H,9H)-dione;
[3a(4)Z]-6-methyl-10,11,13,14-tetrahydro-2H-17,1-(azenometheno)pyrazolo[4,3-m]dipyrrolo[3,2 f:3′,4′-i][1,4]oxazacyclopentadecine-3,8(5H,9H)-dione;
[3a(4)Z]-6-methyl-10,11,13,14-tetrahydro-2H-1,17-(ethanediylidene)pyrazolo[4,3-m]dipyrrolo[3,2-f:3′,4′-i][1,4]oxazacyclopentadecine-3,8(5H,9H)-dione;
[3a(4)Z]-6,9-dimethyl-10,11,13,14-tetrahydro-2H-1,17-(ethanediylidene)pyrazolo[4,3-m]dipyrrolo[3,2-f:3′,4′-i][1,4]oxazacyclopentadecine-3,8(5H,9H)-dione;
[3a(4)Z]-6,9,16-trimethyl-10,11,13,14-tetrahydro-2H-1,17-(ethanediylidene)pyrazolo[4,3-m]dipyrrolo[3,2-f:3′,4′-i][1,4]oxazacyclopentadecine-3,8(5H,9H)-dione;
[3a(4)Z]-6-methyl-9,10,11,12,13,14-hexahydro-17,1-(azenometheno)pyrazolo[3,4-f]dipyrrolo[3,4-j:2′,3′-m][1,4,9]triazacyclopentadecine-3,8(2H,5H)-dione;
[3a(4)Z]-6-methyl-9,10,11,12,13,14-hexahydro-1,17-(ethanediylidene)pyrazolo[3,4-f]dipyrrolo[3,4-j:2′,3′-m][1,4,9]triazacyclopentadecine-3,8(2H,5H)-dione;
[3a(4)Z]-6-methyl-9,10,11,12,13,14-hexahydro-17,1-(azenometheno)pyrazolo[4,3-m]dipyrrolo[3,2-f:3′,4′-i][1,4]diazacyclopentadecine-3,8(2H,5H)-dione;
[3a(4)Z]-6,9-dimethyl-9,10,11,12,13,14-hexahydro-17,1-(azenometheno)pyrazolo[3,4-f]dipyrrolo[3,4-j:2′,3′-m][1,4,9]triazacyclopentadecine-3,8(2H,5H)-dione;
[3a(4)Z]-6,9-dimethyl-9,10,11,12,13,14-hexahydro-1,17-(ethanediylidene)pyrazolo[3,4-f]dipyrrolo[3,4-j:2′,3′-m][1,4,9]triazacyclopentadecine-3,8(2H,5H)-dione;
[3a(4)Z]-6,9-dimethyl-9,10,11,12,13,14-hexahydro-17,1-(azenometheno)pyrazolo[4,3-m]dipyrrolo[3,2-f:3′,4′-i][1,4]diazacyclopentadecine-3,8(2H,5H)-dione;
[3a(4)Z]-6,9-dimethyl-9,10,11,12,13,14-hexahydro-1,17-(ethanediylidene)pyrazolo[4,3-m]dipyrrolo[3,2-f:3′,4′-i][1,4]diazacyclopentadecine-3,8(2H,5H)-dione;
[3a(4)Z]-20-fluoro-6,9-dimethyl-9,10,11,12,13,14-hexahydro-1,17-(ethanediylidene)pyrazolo[4,3-m]dipyrrolo[3,2-f:3′,4′-i][1,4]diazacyclopentadecine-3,8(2H,5H)-dione;
[3a(4)Z]-19-fluoro-6,9-dimethyl-9,10,11,12,13,14-hexahydro-1,17-(ethanediylidene)pyrazolo[4,3-m]dipyrrolo[3,2-f:3′,4′-i][1,4]diazacyclopentadecine-3,8(2H,5H)-dione;
[3a(4)Z]-6,9,20-trimethyl-9,10,11,12,13,14-hexahydro-17,1-(azenometheno)pyrazolo[4,3-m]dipyrrolo[3,2-f:3′,4′-i][1,4]diazacyclopentadecine-3,8(2H,5H)-dione;
[3a(4)Z]-9,20-dimethyl-9,10,11,12,13,14-hexahydro-1,17-(ethanediylidene)pyrazolo[3,4-f]dipyrrolo[3,4-j:2′,3′-m][1,4,9]triazacyclopentadecine-3,8(2H,5H)-dione;
[3a(4)Z]-6,16-dimethyl-9,10,11,12,13,14-hexahydro-17,1-(azenometheno)pyrazolo[4,3-m]dipyrrolo[3,2-f:3′,4′-i][1,4]diazacyclopentadecine-3,8(2H,5H)-dione;
[3a(4)Z]-6,9,16-trimethyl-9,10,11,12,13,14-hexahydro-17,1-(azenometheno)pyrazolo[4,3-m]dipyrrolo[3,2 f:3′,4′-i][1,4]diazacyclopentadecine-3,8(2H,5H)-dione;
[3a(4)Z]-16-cyclopropyl-6,9-dimethyl-9,10,11,12,13,14-hexahydro-17,1-(azenometheno)pyrazolo[4,3-m]dipyrrolo[3,2 f:3′,4′-i][1,4]diazacyclopentadecine-3,8(2H,5H)-dione;
[3a(4)Z]-16-cyclopropyl-6,9-dimethyl-9,10,11,12,13,14-hexahydro-1,17-(ethanediylidene)pyrazolo[3,4-f]dipyrrolo[3,4-j:2′,3′-m][1,4,9]triazacyclopentadecine-3,8(2H,5H)-dione;
[3a(4)Z]-6,9,16-trimethyl-10,11,12,13-tetrahydro-2H-17,1-(azenometheno)[1,2]oxazolo[4,5-m]dipyrrolo[3,2-f:3′,4′-i][1,4]diazacyclopentadecine-3,8(5H,9H)-dione;
[3a(4)Z]-6,9,16-trimethyl-10,11,12,13-tetrahydro-2H-17,1-(azenometheno)[1,2]oxazolo[4,3-m]dipyrrolo[3,2-f:3′,4′-i][1,4]diazacyclopentadecine-3,8(5H,9H)-dione;
[3a(4)Z]-6,14-dimethyl-10,11,13,14-tetrahydro-2H-17,1-(azenometheno)pyrazolo[4,3-m]dipyrrolo[3,2 f:3′,4′-i][1,4]oxazacyclopentadecine-3,8(5H,9H)-dione;
[3a(4)Z]-6,9,14-trimethyl-10,11,13,14-tetrahydro-2H-1,17-(ethanediylidene)pyrazolo[4,3-m]dipyrrolo[3,2-f:3′,4′-i][1,4]oxazacyclopentadecine-3,8(5H,9H)-dione;
[3a(4)Z]-6,9,14-trimethyl-9,10,11,12,13,14-hexahydro-1,17-(ethanediylidene)pyrazolo[4,3-m]dipyrrolo[3,2-f:3′,4′-i][1,4]diazacyclopentadecine-3,8(2H,5H)-dione;
[3a(4)Z]-6,9,12,14-tetramethyl-9,10,11,12,13,14-hexahydro-1,17-(ethanediylidene)pyrazolo[4,3-m]dipyrrolo[3,2-f:3′,4′-i][1,4]diazacyclopentadecine-3,8(2H,5H)-dione;
[3a(4)Z]-6,9,16-trimethyl-9,10,11,12,13,14-hexahydro-1,17-(ethanediylidene)pyrazolo[4,3-m]dipyrrolo[3,2-f:3′,4′-i][1,4]diazacyclopentadecine-3,8(2H,5H)-dione;
[3a(4)Z]-6,9,16-trimethyl-10,11-dihydro-2H,13H-1,17-(ethanediylidene)[1,2]oxazolo[4,3-m]dipyrrolo[3,2-f:3′,4′-i][1,4]oxazacyclopentadecine-3,8(5H,9H)-dione;
[3a(4)Z]-6,9,16-trimethyl-10,11,12,13-tetrahydro-2H-1,17-(ethanediylidene)[1,2]oxazolo[4,3-m]dipyrrolo[3,2-f:3′,4′-i][1,4]diazacyclopentadecine-3,8(5H,9H)-dione;
[3a(4)Z]-6,9,12,14,16-pentamethyl-9,10,11,12,13,14-hexahydro-1,17-(ethanediylidene)pyrazolo[4,3-m]dipyrrolo[3,2-f:3′,4′-i][1,4]diazacyclopentadecine-3,8(2H,5H)-dione;
[3a(4)Z]-6,9,14,16-tetramethyl-9,10,11,12-tetrahydro-14H-1,17-(ethanediylidene)pyrazolo[4,3-n]dipyrrolo[3,2-g:3′,4′-j][1,5]oxazacyclopentadecine-3,8(2H,5H)-dione;
[3a(4)Z]-6,9,14,16-tetramethyl-10,11,13,14-tetrahydro-2H-1,17-(ethanediylidene)pyrazolo[4,3-m]dipyrrolo[3,2-f:3′,4′-i][1,4]oxazacyclopentadecine-3,8(5H,9H)-dione;
[3a(4)Z]-9,14,16-trimethyl-9,10,11,12-tetrahydro-14H-1,17-(ethanediylidene)pyrazolo[4,3-n]dipyrrolo[3,2-g:3′,4′-j][1,5]oxazacyclopentadecine-3,8(2H,5H)-dione;
[3a(4)Z]-9,14,16-trimethyl-10,11,13,14-tetrahydro-2H-1,17-(ethanediylidene)pyrazolo[4,3-m]dipyrrolo[3,2-f:3′,4′-i][1,4]oxazacyclopentadecine-3,8(5H,9H)-dione;
[3a(4)Z]-12-ethyl-6,9,14-trimethyl-9,10,11,12,13,14-hexahydro-1,17-(ethanediylidene)pyrazolo[4,3-m]dipyrrolo[3,2-f:3′,4′-i][1,4]diazacyclopentadecine-3,8(2H,5H)-dione;
[3a(4)Z]-6,9,14-trimethyl-12-(propan-2-yl)-9,10,11,12,13,14-hexahydro-1,17-(ethanediylidene)pyrazolo[4,3-m]dipyrrolo[3,2-f:3′,4′-i][1,4]diazacyclopentadecine-3,8(2H,5H)-dione;
[3a(4)Z]-16-cyclopropyl-6,9-dimethyl-10,11-dihydro-2H,13H-1,17-(ethanediylidene)[1,2]oxazolo[4,3-m]dipyrrolo[3,2-f:3′,4′-i][1,4]oxazacyclopentadecine-3,8(5H,9H)-dione;
[3a(4)Z]-9,14-dimethyl-9,10,11,12,13,14-hexahydro-1,17-(ethanediylidene)pyrazolo[4,3-m]dipyrrolo[3,2-f:3′,4′-i][1,4]diazacyclopentadecine-3,8(2H,5H)-dione;
[3a(4)Z]-6,9-dimethyl-16-(propan-2-yl)-10,11-dihydro-2H,13H-1,17-(ethanediylidene)[1,2]oxazolo[4,3-m]dipyrrolo[3,2-f:3′,4′-i][1,4]oxazacyclopentadecine-3,8(5H,9H)-dione;
[3a(4)Z]-9-methyl-16-(propan-2-yl)-10,11-dihydro-2H,13H-1,17-(ethanediylidene)[1,2]oxazolo[4,3-m]dipyrrolo[3,2-f:3′,4′-i][1,4]oxazacyclopentadecine-3,8(5H,9H)-dione;
[3a(4)Z]-6,9,14-trimethyl-9,10,11,12-tetrahydro-14H-1,17-(ethanediylidene)pyrazolo[4,3-n]dipyrrolo[3,2-g:3′,4′-j][1,5]oxazacyclopentadecine-3,8(2H,5H)-dione;
[3a(4)Z]-9,14-dimethyl-9,10,11,12-tetrahydro-14H-1,17-(ethanediylidene)pyrazolo[4,3-n]dipyrrolo[3,2-g:3′,4′-j][1,5]oxazacyclopentadecine-3,8(2H,5H)-dione;
[3a(4)Z]-6,9,12,14-tetramethyl-9,10,11,12,13,14-hexahydro-17,1-(azenometheno)pyrazolo[4,3-m]dipyrrolo[3,2-f:3′,4′-i][1,4]diazacyclopentadecine-3,8(2H,5H)-dione;
[3a(4)Z]-9,12,14-trimethyl-9,10,11,12,13,14-hexahydro-17,1-(azenometheno)pyrazolo[4,3-m]dipyrrolo[3,2 f:3′,4′-i][1,4]diazacyclopentadecine-3,8(2H,5H)-dione;
[3a(4)Z]-6,9,12,14-tetramethyl-9,10,11,12,13,14-hexahydro-1,17-(ethanediylidene)pyrazolo[3,4-f]dipyrrolo[3,4-j:2′,3′-m][1,4,9]triazacyclopentadecine-3,8(2H,5H)-dione; and
[3a(4)Z]-9,12,14-trimethyl-9,10,11,12,13,14-hexahydro-1,17-(ethanediylidene)pyrazolo[3,4-f]dipyrrolo[3,4-j:2′,3′-m][1,4,9]triazacyclopentadecine-3,8(2H,5H)-dione.

60. The compound of clause 1, or a pharmaceutically acceptable salt thereof, selected from the group consisting of [3a(4)Z]-6-methyl-9,10,11,12-tetrahydro-1,18-(ethanediylidene)dipyrrolo[3,2-g:3′,4′-j][1,5,12]benzoxadiazacyclopentadecine-3,8(2H,5H)-dione;
[3a(4)Z]-6-methyl-10,11-dihydro-2H-1,17-(ethanediylidene)dipyrrolo[3,2-f:3′,4′-i][1,4,11]benzoxadiazacyclotetradecine-3,8(5H,9H)-dione;
[3a(4)Z]-6-methyl-10,11-dihydro-2H-17,1-(azenometheno)dipyrrolo[3,2-f:3′,4′-i][1,4]benzoxazacyclotetradecine-3,8(5H,9H)-dione;
[3a(4)Z]-16-fluoro-6-methyl-10,11-dihydro-2H-1,17-(ethanediylidene)dipyrrolo[3,2-f:3′,4′-i][1,4,11]benzoxadiazacyclotetradecine-3,8(5H,9H)-dione;
[3a(4)Z]-15-fluoro-6-methyl-10,11-dihydro-2H-1,17-(ethanediylidene)dipyrrolo[3,2-f:3′,4′-i][1,4,11]benzoxadiazacyclotetradecine-3,8(5H,9H)-dione;
[3a(4)Z]-14-fluoro-6-methyl-10,11-dihydro-2H-1,17-(ethanediylidene)dipyrrolo[3,2-f:3′,4′-i][1,4,11]benzoxadiazacyclotetradecine-3,8(5H,9H)-dione;
[3a(4)Z]-13-fluoro-6-methyl-10,11-dihydro-2H-1,17-(ethanediylidene)dipyrrolo[3,2-f:3′,4′-i][1,4,11]benzoxadiazacyclotetradecine-3,8(5H,9H)-dione; and
[3a(4)Z]-6,9,12-trimethyl-10,11,12,13-tetrahydro-2H-1,18-(ethanediylidene)dipyrrolo[3,2-g:3′,4′-j][2,5]benzodiazacyclopentadecine-3,8(5H,9H)-dione.

61. The compound of clause 1, or a pharmaceutically acceptable salt thereof, selected from the group consisting of [3a(4)Z]-6-methyl-10,11-dihydro-2H-1,17-(ethanediylidene)pyrido[3,2-m]dipyrrolo[3,2-f:3′,4′-i][1,4,11]oxadiazacyclotetradecine-3,8(5H,9H)-dione;
[3a(4)Z]-6-methyl-10,11-dihydro-2H-1,17-(ethanediylidene)pyrimido[5,4-m]dipyrrolo[3,2-f:3′,4′-i][1,4,11]oxadiazacyclotetradecine-3,8(5H,9H)-dione;
[3a(4)Z]-6,16-dimethyl-10,11-dihydro-2H-1,17-(ethanediylidene)pyrido[3,4-m]dipyrrolo[3,2-f:3′,4′-i][1,4,11]oxadiazacyclotetradecine-3,8(5H,9H)-dione;
[3a(4)Z]-6-methyl-9,10,11,12-tetrahydro-14H-1,18-(ethanediylidene)pyrido[2,1-c]dipyrrolo[3,2-j:3′,4′-m][1,4,8]triazacyclotetradecine-3,8,14(2H,5H)-trione;
[3a(4)Z]-6-methyl-9,10,11,12-tetrahydro-14H-18,1-(azenometheno)pyrido[1,2-e]dipyrrolo[3,4-i:2′,3′-l][1,5]diazacyclotetradecine-3,8,14(2H,5H)-trione;
   or a pharmaceutically acceptable salt thereof.

62. The compound of clause 1, or a pharmaceutically acceptable salt thereof, selected from the group consisting of [3a(4)Z,13aR]-6-methyl-10,11,12,13,13a,14,15,16-octahydro-2H-18,1-(azenometheno)tripyrrolo[1,2-a:3′,2′-i:3″,4″-][1,4,7]triazacyclopentadecine-3,8(5H,9H)-dione;
[3a(4)Z,13aR]-6-methyl-9,10,11,12,13,13a,14,15-octahydro-17,1-(azenometheno)azeto[1,2-a]dipyrrolo[3,2-i:3′,4′-l][1,4,7]triazacyclopentadecine-3,8(2H,5H)-dione;
[16a(17)Z]-2,11-dimethyl-6,7,10,11-tetrahydro-1H,9H-12,14-(azenometheno)dipyrrolo[3,4-g:2′,3′-j][1,4,6,13]oxatriazacyclopentadecine-4,16(5H,15H)-dione;
[16a(17)Z]-2,5,11-trimethyl-6,7,10,11-tetrahydro-1H,9H-12,14-(azenometheno)dipyrrolo[3,2-f:3′,4′-i][1,4,13]oxadiazacyclopentadecine-4,16(5H,15H)-dione;
[17a(18)Z]-2,12-dimethyl-6,7,9,10,11,12-hexahydro-1H-13,15-(azenometheno)dipyrrolo[3,2-f:3′,4′-i][1,4,13]oxadiazacyclohexadecine-4,17(5H,16H)-dione;
[17a(18)Z]-2,5,12-trimethyl-6,7,9,10,11,12-hexahydro-1H-13,15-(azenometheno)dipyrrolo[3,2-f:3′,4′-i][1,4,13]oxadiazacyclohexadecine-4,17(5H,16H)-dione;
[17a(18)Z]-2,5,12-trimethyl-6,7,9,10,11,12-hexahydro-1H-13,15-(azenometheno)dipyrrolo[3,2-f:3′,4′-i][1,4,11,13]oxatriazacyclohexadecine-4,17(5H,16H)-dione;
[16a(17)Z]-2,5,11-trimethyl-6,7,8,9,10,11-hexahydro-1H-12,14-(azenometheno)dipyrrolo[3,2-i:3′,4′-l][1,4,7]triazacyclopentadecine-4,16(5H,15H)-dione;
[16a(17)Z]-2,5,11-trimethyl-6,7,8,9,10,11-hexahydro-1H-12,14-(ethanediylidene)dipyrrolo[3,2-k:3′,4′-n][1,3,6,9]tetraazacyclopentadecine-4,16(5H,15H)-dione;
[16a(17)Z]-2,5,11-trimethyl-6,7,8,9,10,11-hexahydro-1H-12,14-(azenometheno)dipyrrolo[3,2-k:3′,4′-n][1,3,6,9]tetraazacyclopentadecine-4,16(5H,15H)-dione;
[16a(17)Z]-11-cyclopropyl-2,5-dimethyl-6,7,8,9,10,11-hexahydro-1H-12,14-(ethanediylidene)dipyrrolo[3,2-k:3′,4′-n][1,3,6,9]tetraazacyclopentadecine-4,16(5H,15H)-dione;
[16a(17)Z]-11-cyclopropyl-2-methyl-6,7,8,9,10,11-hexahydro-1H-12,14-(azenometheno)dipyrrolo[3,2-k:3′,4′-n][1,3,6,9]tetraazacyclopentadecine-4,16(5H,15H)-dione;
[10R,16a(17)Z]-2,5,10-trimethyl-6,7,8,9,10,11-hexahydro-1H-12,14-(azenometheno)dipyrrolo[3,2-i:3′,4′-l][1,4,7]triazacyclopentadecine-4,16(5H,15H)-dione;
[10S,16a(17)Z]-2,5,10-trimethyl-6,7,8,9,10,11-hexahydro-1H-12,14-(azenometheno)dipyrrolo[3,2-i:3′,4′-l][1,4,7]triazacyclopentadecine-4,16(5H,15H)-dione;
[10S,16a(17)Z]-2,5,10-trimethyl-6,7,10,11-tetrahydro-1H,9H-12,14-(azenometheno)dipyrrolo[3,2-f:3′,4′-i][1,4,13]oxadiazacyclopentadecine-4,16(5H,15H)-dione;
[10S,16a(17)Z]-2,5,10-trimethyl-6,7,10,11-tetrahydro-1H,9H-12,14-(azenometheno)dipyrrolo[3,4-g:2′,3′-j][1,4,6,13]oxatriazacyclopentadecine-4,16(5H,15H)-dione;
[10S,16a(17)Z]-2,5,10-trimethyl-6,7,9,10-tetrahydro-1H-12,14-(azenometheno)dipyrrolo[3,2-i:3′,4′-l][1,4,7]dioxazacyclopentadecine-4,16(5H,15H)-dione;
[10S,16a(17)Z]-2,5,10-trimethyl-6,7,9,10-tetrahydro-1H-12,14-(azenometheno)dipyrrolo[3,4-d:2′,3′-g][1,13,3,10]dioxadiazacyclopentadecine-4,16(5H,15H)-dione;
[10S,16a(17)Z]-2,5,10-trimethyl-6,7,9,10-tetrahydro-1H-12,14-(ethanediylidene)dipyrrolo[3,4-d:2′,3′-g][1,13,3,10]dioxadiazacyclopentadecine-4,16(5H,15H)-dione;
[10S,16a(17)Z]-2,5,10-trimethyl-5,6,7,8,9,10-hexahydro-12,14-(azenometheno)dipyrrolo[3,4-d:2′,3′-g][1,3,10,13]oxatriazacyclopentadecine-4,16(1H,15H)-dione;
[10S,16a(17)Z]-2,5,10-trimethyl-5,6,7,8,9,10-hexahydro-12,14-(ethanediylidene)dipyrrolo[3,4-d:2′,3′-g][1,3,10,13]oxatriazacyclopentadecine-4,16(1H,15H)-dione;
[10S,16a(17)Z]-2,5,10-trimethyl-5,6,7,8,9,10-hexahydro-12,14-(azenometheno)dipyrrolo[3,2-i:3′,4′-l][1,4,7]oxadiazacyclopentadecine-4,16(1H,15H)-dione;
[9R,16a(17)Z]-2,5,9-trimethyl-6,7,9,10-tetrahydro-1H-12,14-(azenometheno)dipyrrolo[3,4-d:2′,3′-g][1,13,3,10]dioxadiazacyclopentadecine-4,16(5H,15H)-dione;
[9S,16a(17)Z]-2,5,9-trimethyl-6,7,9,10-tetrahydro-1H-12,14-(azenometheno)dipyrrolo[3,4-d:2′,3′-g][1,13,3,10]dioxadiazacyclopentadecine-4,16(5H,15H)-dione;
[16a(17)Z]-2,5-dimethyl-6,7,9,10-tetrahydro-1H-12,14-(ethanediylidene)dipyrrolo[3,2-i:3′,4′-][1,4,7]dioxazacyclopentadecine-4,16(5H,15H)-dione;
[10S,16a(17)Z]-2,5,10-trimethyl-6,7,9,10-tetrahydro-1H-12,14-(ethanediylidene)dipyrrolo[3,2-i:3′,4′-l][1,4,7]dioxazacyclopentadecine-4,16(5H,15H)-dione;
[10R,16a(17)Z]-2,5,10-trimethyl-6,7,9,10-tetrahydro-1H-12,14-(ethanediylidene)dipyrrolo[3,2-i:3′,4′-l][1,4,7]dioxazacyclopentadecine-4,16(5H,15H)-dione;
[10S,16a(17)Z]-2,10-dimethyl-6,7,9,10-tetrahydro-1H-12,14-(ethanediylidene)dipyrrolo[3,2-i:3′,4′-l][1,4,7]dioxazacyclopentadecine-4,16(5H,15H)-dione;
[10S,16a(17)Z]-2,5,10-trimethyl-5,6,7,8,9,10-hexahydro-12,14-(ethanediylidene)dipyrrolo[3,2-i:3′,4′-l][1,4,7]oxadiazacyclopentadecine-4,16(1H,15H)-dione;
[9R,16a(17)Z]-2,5,9-trimethyl-6,7,9,10-tetrahydro-1H-12,14-(ethanediylidene)dipyrrolo[3,2-i:3′,4′-l][1,4,7]dioxazacyclopentadecine-4,16(5H,15H)-dione;
[9S,16a(17)Z]-2,5,9-trimethyl-6,7,9,10-tetrahydro-1H-12,14-(ethanediylidene)dipyrrolo[3,2-i:3′,4′-l][1,4,7]dioxazacyclopentadecine-4,16(5H,15H)-dione;
[17a(18)Z]-2-methyl-6,7,10,11-tetrahydro-1H,9H-13,15-(ethanediylidene)dipyrrolo[3,2-f:3′,4′-i][1,13,4]oxathiazacyclohexadecine-4,17(5H,16H)-dione;
[17a(18)Z]-2-methyl-6,7,10,11-tetrahydro-1H-13,15-(ethanediylidene)-12⁶-dipyrrolo[3,2-f:3′,4′-i][1,13,4]oxathiazacyclohexadecine-4,12,12,17(5H,9H,16H)-tetrone;
[17a(18)Z]-2-methyl-6,7,9,10-tetrahydro-1H,12H-13,15-(ethanediylidene)dipyrrolo[3,2-i:3′,4′-l][1,4,7]dioxazacyclohexadecine-4,17(5H,16H)-dione;
[12R,17a(18)Z]-2,12-dimethyl-6,7,9,10-tetrahydro-1H,12H-13,15-(ethanediylidene)dipyrrolo[3,2-i:3′,4′-][1,4,7]dioxazacyclohexadecine-4,17(5H,16H)-dione;
[12S,17a(18)Z]-2,12-dimethyl-6,7,9,10-tetrahydro-1H,12H-13,15-(ethanediylidene)dipyrrolo[3,2-i:3′,4′-][1,4,7]dioxazacyclohexadecine-4,17(5H,16H)-dione;
[12S,17a(18)Z]-2,5,12-trimethyl-6,7,9,10-tetrahydro-1H,12H-13,15-(ethanediylidene)dipyrrolo[3,2-i:3′,4′-l][1,4,7]dioxazacyclohexadecine-4,17(5H,16H)-dione;
[17a(18)Z]-2,5-dimethyl-6,7,11,12-tetrahydro-1H-13,15-(ethanediylidene)dipyrrolo[3,2-f:3′,4′-i][1,4,14]oxadiazacyclohexadecine-4,10,17(5H,9H,16H)-trione;
[17a(18)Z]-2,5-dimethyl-6,7,11,12-tetrahydro-1H-13,15-(azenometheno)dipyrrolo[3,4-h:2′,3′-k][1,4,7,14]oxatriazacyclohexadecine-4,10,17(5H,9H,16H)-trione;
[17a(18)Z]-2,5-dimethyl-6,7,11,12-tetrahydro-1H-13,15-(azenometheno)dipyrrolo[3,2 f:3′,4′-i][1,4,14]oxadiazacyclohexadecine-4,10,17(5H,9H,16H)-trione;
[17a(18)Z]-2,5-dimethyl-6,7,11,12-tetrahydro-1H-13,15-(ethanediylidene)dipyrrolo[3,4-h:2′,3′-k][1,4,7,14]oxatriazacyclohexadecine-4,10,17(5H,9H,16H)-trione;
[12S,17a(18)Z]-2,5,12-trimethyl-6,7,11,12-tetrahydro-1H-13,15-(ethanediylidene)dipyrrolo[3,4-h:2′,3′-k][1,4,7,14]oxatriazacyclohexadecine-4,10,17(5H,9H,16H)-trione;
[17a(18)Z]-2-methyl-6,7,11,12-tetrahydro-1H-13,15-(ethanediylidene)dipyrrolo[3,4-h:2′,3′-k][1,4,7,14]oxatriazacyclohexadecine-4,10,17(5H,9H,16H)-trione;
[17a(18)Z]-2,11-dimethyl-6,7,11,12-tetrahydro-1H-13,15-(ethanediylidene)dipyrrolo[3,4-h:2′,3′-k][1,4,7,14]oxatriazacyclohexadecine-4,10,17(5H,9H,16H)-trione;
[17a(18)Z]-2,11-dimethyl-6,7,11,12-tetrahydro-1H-13,15-(ethanediylidene)dipyrrolo[3,2 f:3′,4′-i][1,4,14]oxadiazacyclohexadecine-4,10,17(5H,9H,16H)-trione;
[17a(18)Z]-2,11-dimethyl-6,7,11,12-tetrahydro-1H-13,15-(azenometheno)dipyrrolo[3,4-h:2′,3′-k][1,4,7,14]oxatriazacyclohexadecine-4,10,17(5H,9H,16H)-trione;
[17a(18)Z]-2,11-dimethyl-6,7,11,12-tetrahydro-1H-13,15-(azenometheno)dipyrrolo[3,2-f:3′,4′-i][1,4,14]oxadiazacyclohexadecine-4,10,17(5H,9H,16H)-trione;
[18a(19)Z]-2-methyl-6,7,10,11-tetrahydro-1H,9H-14,16-(ethanediylidene)dipyrrolo[3,2 f:3′,4′-i][1,4,15]oxadiazacycloheptadecine-4,12,18(5H,13H,17H)-trione;
[18a(19)Z]-2,5-dimethyl-6,7,10,11-tetrahydro-1H,9H-14,16-(ethanediylidene)dipyrrolo[3,2 f:3′,4′-i][1,4,15]oxadiazacycloheptadecine-4,12,18(5H,13H,17H)-trione;
[18a(19)Z]-2,11-dimethyl-6,7,10,11-tetrahydro-1H,9H-14,16-(ethanediylidene)dipyrrolo[3,2-f:3′,4′-i][1,4,15]oxadiazacycloheptadecine-4,12,18(5H,13H,17H)-trione;
[13S,18a(19)Z]-2,13-dimethyl-6,7,10,11-tetrahydro-1H,9H-14,16-(ethanediylidene)dipyrrolo[3,2-f:3′,4′-i][1,4,15]oxadiazacycloheptadecine-4,12,18(5H,13H,17H)-trione;
[13R,18a(19)Z]-2,13-dimethyl-6,7,10,11-tetrahydro-1H,9H-14,16-(ethanediylidene)dipyrrolo[3,2-f:3′,4′-i][1,4,15]oxadiazacycloheptadecine-4,12,18(5H,13H,17H)-trione;
[18a(19)Z]-2-methyl-6,7,10,11-tetrahydro-1H,9H-14,16-(azenometheno)dipyrrolo[3,4-i:2′,3′-1][1,4,8,15]oxatriazacycloheptadecine-4,12,18(5H,13H,17H)-trione;
[13S,18a(19)Z]-2,13-dimethyl-6,7,10,11-tetrahydro-1H,9H-14,16-(ethanediylidene)dipyrrolo[3,4-i:2′,3′-][1,4,8,15]oxatriazacycloheptadecine-4,12,18(5H,13H,17H)-trione;
[13S,18a(19)Z]-2,13-dimethyl-6,7,10,11-tetrahydro-1H,9H-14,16-(azenometheno)dipyrrolo[3,2-f:3′,4′-i][1,4,15]oxadiazacycloheptadecine-4,12,18(5H,13H,17H)-trione;
[13S,18a(19)Z]-13-hydroxy-2,13-dimethyl-6,7,10,11-tetrahydro-1H,9H-14,16-(ethanediylidene)dipyrrolo[3,2 f:3′,4′-i][1,4,15]oxadiazacycloheptadecine-4,12,18(5H,13H,17H)-trione;
[16a(17)Z]-2-methyl-6,7,9,10-tetrahydro-1H-12,14-(ethanediylidene)dipyrrolo[3,2-i:3′,4′-l][1,4,7]dioxazacyclopentadecine-4,16(5H,15H)-dione;
[16a(17)Z]-19-chloro-2-methyl-6,7,9,10-tetrahydro-1H-12,14-(ethanediylidene)dipyrrolo[3,2-i:3′,4′-l][1,4,7]dioxazacyclopentadecine-4,16(5H,15H)-dione;
[16a(17)Z]-19-chloro-2,5-dimethyl-6,7,9,10-tetrahydro-1H-12,14-(ethanediylidene)dipyrrolo[3,2-i:3′,4′-l][1,4,7]dioxazacyclopentadecine-4,16(5H,15H)-dione;
methyl[7R,16a(17)Z]-19-chloro-2,5-dimethyl-4,16-dioxo-4,5,6,7,9,10,15,16-octahydro-1H-12,14-(ethanediylidene)dipyrrolo[3,2-i:3′,4′-l][1,4,7]dioxazacyclopentadecine-7-carboxylate;
[7R,16a(17)Z]—N-(azetidin-3-yl)-19-chloro-2,5-dimethyl-4,16-dioxo-4,5,6,7,9,10,15,16-octahydro-1H-12,14-(ethanediylidene)dipyrrolo[3,2-i:3′,4′-l][1,4,7]dioxazacyclopentadecine-7-carboxamide;
[7R,16a(17)Z]-19-chloro-2,5-dimethyl-4,16-dioxo-N-(piperidin-4-yl)-4,5,6,7,9,10,15,16-octahydro-1H-12,14-(ethanediylidene)dipyrrolo[3,2-i:3′,4′-l][1,4,7]dioxazacyclopentadecine-7-carboxamide;
[7R,16a(17)Z]-19-chloro-N,2,5-trimethyl-4,16-dioxo-4,5,6,7,9,10,15,16-octahydro-1H-12,14-(ethanediylidene)dipyrrolo[3,2-i:3′,4′-l][1,4,7]dioxazacyclopentadecine-7-carboxamide;
[7R,16a(17)Z]-19-chloro-2,5-dimethyl-4,16-dioxo-N-[(3R)-pyrrolidin-3-yl]-4,5,6,7,9,10,15,16-octahydro-1H-12,14-(ethanediylidene)dipyrrolo[3,2-i:3′,4′-l][1,4,7]dioxazacyclopentadecine-7-carboxamide;
[7R,16a(17)Z]-19-chloro-N,N,2,5-tetramethyl-4,16-dioxo-4,5,6,7,9,10,15,16-octahydro-1H-12,14-(ethanediylidene)dipyrrolo[3,2-i:3′,4′-1][1,4,7]dioxazacyclopentadecine-7-carboxamide;
[7R,16a(17)Z]-19-chloro-2,5-dimethyl-7-(4-methylpiperazine-1-carbonyl)-6,7,9,10-tetrahydro-1H-12,14-(ethanediylidene)dipyrrolo[3,2-i:3′,4′-l][1,4,7]dioxazacyclopentadecine-4,16(5H,15H)-dione;
[10S,16a(17)Z]-2,5,10-trimethyl-6,7,9,10-tetrahydro-1H-12,14-(ethanediylidene)dipyrrolo[3,2-i:3′,4′-l][1,4,7]dioxazacyclopentadecine-4,16(5H,15H)-dione;
[10S,16a(17)Z]-19-chloro-2,5,10-trimethyl-6,7,9,10-tetrahydro-1H-12,14-(ethanediylidene)dipyrrolo[3,2-i:3′,4′-l][1,4,7]dioxazacyclopentadecine-4,16(5H,15H)-dione;
[16a(17)Z]-19-chloro-5-methyl-6,7,9,10-tetrahydro-1H-12,14-(ethanediylidene)dipyrrolo[3,2-i:3′,4′-l][1,4,7]dioxazacyclopentadecine-4,16(5H,15H)-dione;
[16a(17)Z]-19-chloro-2,5-dimethyl-6,7,9,10-tetrahydro-1H-12,14-(ethanediylidene)dipyrrolo[3,2-i:3′,4′-l][1,4,7]oxathiazacyclopentadecine-4,16(5H,15H)-dione;
[16a(17)Z]-19-chloro-2,5-dimethyl-5,6,7,8,9,10-hexahydro-12,14-(ethanediylidene)dipyrrolo[3,2-i:3′,4′-l][1,4,7]oxadiazacyclopentadecine-4,16(1H,15H)-dione;
[16a(17)Z]-19-chloro-2,5-dimethyl-6,7,9,10-tetrahydro-12,14-(ethanediylidene)-8λ⁶-dipyrrolo[3,2-i:3′,4′-l][1,4,7]oxathiazacyclopentadecine-4,8,8,16(1H,5H,15H)-tetrone;
[16a(17)Z]-19-chloro-2,5-dimethyl-6,7,9,10-tetrahydro-12,14-(ethanediylidene)-8λ⁴-dipyrrolo[3,2-i:3′,4′-l][1,4,7]oxathiazacyclopentadecine-4,8,16(1H,5H,15H)-trione;
[16a(17)Z]-19-chloro-5-methyl-6,7,9,10-tetrahydro-1H-12,14-(ethanediylidene)dipyrrolo[3,2-i:3′,4′-l][1,4,7]oxathiazacyclopentadecine-4,16(5H,15H)-dione;
[16a(17)Z]-2,5-dimethyl-6,7,9,10-tetrahydro-1H-12,14-(ethanediylidene)dipyrrolo[3,4-g:2′,3′-j][1,4,13]oxathiazacyclopentadecine-4,16(5H,15H)-dione;
[16a(17)Z]-2,5-dimethyl-6,7-dihydro-1H,9H-12,14-(ethanediylidene)-11λ⁶-dipyrrolo[3,4-g:2′,3′-j][1,4,13]oxathiazacyclopentadecine-4,11,11,16(5H,10H,15H)-tetrone;
[16a(17)Z]-5-methyl-6,7,9,10-tetrahydro-1H-12,14-(ethanediylidene)dipyrrolo[3,4-g:2′,3′-j][1,4,13]oxathiazacyclopentadecine-4,16(5H,15H)-dione;
[16a(17)Z]-19-chloro-5-methyl-6,7,9,10-tetrahydro-1H-12,14-(ethanediylidene)pyrazolo[4,3-i]pyrrolo[3,4-1][1,4,7]dioxazacyclopentadecine-4,16(5H,15H)-dione;
[16a(17)Z]-19-chloro-5-methyl-5,6,7,8,9,10-hexahydro-12,14-(ethanediylidene)dipyrrolo[3,2-i:3′,4′-l][1,4,7]oxadiazacyclopentadecine-4,16(1H,15H)-dione;
[16a(17)Z]-19-chloro-2,5,8-trimethyl-5,6,7,8,9,10-hexahydro-12,14-(ethanediylidene)dipyrrolo[3,2-i:3′,4′-l][1,4,7]oxadiazacyclopentadecine-4,16(1H,15H)-dione;
[16a(17)Z]-2,5-dimethyl-4,16-dioxo-4,5,6,7,9,10,15,16-octahydro-1H-12,14-(ethanediylidene)dipyrrolo[3,2-i:3′,4′-l][1,4,7]dioxazacyclopentadecine-19-carbonitrile;
[16a(17)Z]-19-chloro-2,5-dimethyl-6,7,9,10-tetrahydro-1H-12,14-(ethanediylidene)dipyrrolo[3,4-g:2′,3′-j][1,4,13]oxathiazacyclopentadecine-4,16(5H,15H)-dione;
[16a(17)Z]-19-chloro-5-methyl-6,7,9,10-tetrahydro-1H-12,14-(ethanediylidene)dipyrrolo[3,4-g:2′,3′-j][1,4,13]oxathiazacyclopentadecine-4,16(5H,15H)-dione; and

[16a(17)Z]-19-chloro-5-methyl-6,7-dihydro-1H,9H-12,14-(ethanediylidene)-11λ⁶-dipyrrolo[3,4-g:2′,3′-j][1,4,13]oxathiazacyclopentadecine-4,11,11,16(5H,10H,15H)-tetrone.

63. A pharmaceutical composition comprising at least one compound of any one of clauses 1 to 62, or a pharmaceutically acceptable salt thereof, and optionally one or more pharmaceutically acceptable excipients.

64. A method of treating disease, such as cancer, comprising administering to a subject in need of such treatment an effective amount of a compound of any one of clauses 1 to 62, or a pharmaceutically acceptable salt thereof.

65. A compound of any one of clauses 1 to 62, or a pharmaceutically acceptable salt thereof, for use in a method of treating cancer in a subject.

66. A compound of any one of clauses 1 to 62, or a pharmaceutically acceptable salt thereof, for treating cancer in a subject.

67. Use of a compound of any one of clauses 1 to 62, or a pharmaceutically acceptable salt thereof, in the manufacture of a medicament for treating cancer in a subject.

### DETAILED DESCRIPTION

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

For the sake of brevity, the disclosures of the publications cited in this specification, including patents, are herein incorporated by reference. Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As used herein, the terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001.

Chemical nomenclature for compounds described herein has generally been derived using the commercially-available ACD/Name 2014 (ACD/Labs) or ChemBioDraw Ultra 13.0 (Perkin Elmer).

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present disclosure and are disclosed herein just as if each and every such sub-combination of chemical groups was individually and explicitly disclosed herein.

### Chemical Definitions

The term “alkyl” refers to a straight- or branched-chain mono-valent hydrocarbon group. The term “alkylene” refers to a straight- or branched-chain di-valent hydrocarbon group. In some embodiments, it can be advantageous to limit the number of atoms in an “alkyl” or “alkylene” to a specific range of atoms, such as C₁-C₂₀ alkyl or C₁-C₂₀ alkylene, C₁-C₁₂ alkyl or C₁-C₁₂ alkylene, or C₁-C₆ alkyl or C₁-C₆ alkylene. Examples of alkyl groups include methyl (Me), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples. Examples of alkylene groups include methylene (—CH₂—), ethylene ((—CH₂—)₂), n-propylene ((—CH₂—)₃), iso-propylene ((—C(H)(CH₃)CH₂—)), n-butylene ((—CH₂—)₄), and the like. It will be appreciated that an alkyl or alkylene group can be unsubstituted or substituted as described herein. An alkyl or alkylene group can be substituted with any of the substituents in the various embodiments described herein, including one or more of such substituents.

The term “alkenyl” refers to a straight- or branched-chain mono-valent hydrocarbon group having one or more double bonds. The term “alkenylene” refers to a straight- or branched-chain di-valent hydrocarbon group having one or more double bonds. In some embodiments, it can be advantageous to limit the number of atoms in an “alkenyl” or “alkenylene” to a specific range of atoms, such as C₂-C₂₀ alkenyl or C₂-C₂₀ alkenylene, C₂-C₁₂ alkenyl or C₂-C₁₂ alkenylene, or C₂-C₆ alkenyl or C₂-C₆ alkenylene. Examples of alkenyl groups include ethenyl (or vinyl), allyl, and but-3-en-1-yl. Examples of alkenylene groups include ethenylene (or vinylene) (—CH═CH—), n-propenylene (—CH═CHCH₂—), iso-propenylene (—CH═CH(CH₃)—), and and the like. Included within this term are cis and trans isomers and mixtures thereof. It will be appreciated that an alkenyl or alkenylene group can be unsubstituted or substituted as described herein. An alkenyl or alkenylene group can be substituted with any of the substituents in the various embodiments described herein, including one or more of such substituents.

The term “alkynyl” refers to a straight- or branched-chain mono-valent hydrocarbon group having one or more triple bonds. The term “alkynylene” refers to a straight- or branched-chain di-valent hydrocarbon group having one or more triple bonds. In some embodiments, it can be advantageous to limit the number of atoms in an “alkynyl” or “alkynylene” to a specific range of atoms, such as C₂-C₂₀ alkynyl or C₂-C₂₀ alkynylene, C₂-C₁₂ alkynyl or C₂-C₁₂ alkynylene, or C₂-C₆ alkynyl or C₂-C₆ alkynylene. Examples of alkynyl groups include acetylenyl (—C≡CH) and propargyl (—CH₂C≡CH), but-3-yn-1,4-diyl (—C≡C—CH₂CH₂—), and the like. It will be appreciated that an alkynyl or alkynylene group can be unsubstituted or substituted as described herein. An alkynyl or alkynylene group can be substituted with any of the substituents in the various embodiments described herein, including one or more of such substituents.

The term “cycloalkyl” refers to a saturated or partially saturated, monocyclic or polycyclic mono-valent carbocycle. The term “cycloalkylene” refers to a saturated or partially saturated, monocyclic or polycyclic di-valent carbocycle. In some embodiments, it can be advantageous to limit the number of atoms in a “cycloalkyl” or “cycloalkylene” to a specific range of atoms, such as having 3 to 12 ring atoms. Polycyclic carbocycles include fused, bridged, and spiro polycyclic systems. Illustrative examples of cycloalkyl groups include mono-valent radicals of the following entities, while cycloalkylene groups include di-valent radicals of the following entities, in the form of properly bonded moieties:

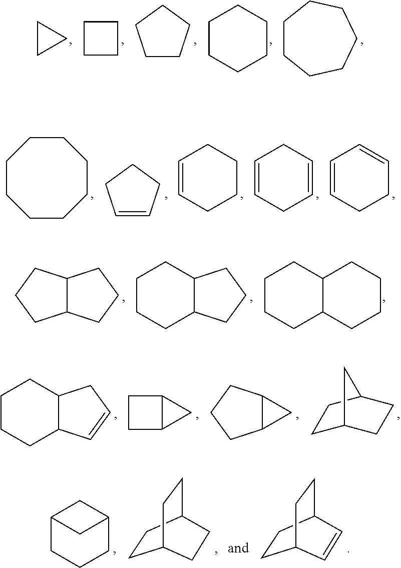

In particular, a cyclopropyl moiety can be depicted by the structural formula

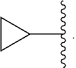 [SMILES: *C1CC1]

In particular, a cyclopropylene moiety can be depicted by the structural formula

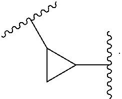 [SMILES: *C1CC1*]

It will be appreciated that a cycloalkyl or cycloalkylene group can be unsubstituted or substituted as described herein. A cycloalkyl or cycloalkylene group can be substituted with any of the substituents in the various embodiments described herein, including one or more of such substituents.

The term “halogen” or “halo” represents chlorine, fluorine, bromine, or iodine.

The term “haloalkyl” refers to an alkyl group with one or more halo substituents. Examples of haloalkyl groups include —CF₃, —(CH₂)F, —CHF₂, —CH₂Br, —CH₂CF₃, and —CH₂CH₂F. The term “haloalkylene” refers to an alkyl group with one or more halo substituents. Examples of haloalkyl groups include —CF₂—, —C(H)(F)—, —C(H)(Br)—, —CH₂CF₂—, and —CH₂C(H)(F)—.

The term “aryl” refers to a mono-valent all-carbon monocyclic or fused-ring polycyclic group having a completely conjugated pi-electron system. The term “arylene” refers to a mono-valent all-carbon monocyclic or fused-ring polycyclic group having a completely conjugated pi-electron system. In some embodiments, it can be advantageous to limit the number of atoms in an “aryl” or “arylene” to a specific range of atoms, such as mono-valent all-carbon monocyclic or fused-ring polycyclic groups of 6 to 14 carbon atoms (C₆-C₁₄ aryl), mono-valent all-carbon monocyclic or fused-ring polycyclic groups of 6 to 10 carbon atoms (C₆-C₁₀ aryl), di-valent all-carbon monocyclic or fused-ring polycyclic groups of 6 to 14 carbon atoms (C₆-C₁₄ arylene), di-valent all-carbon monocyclic or fused-ring polycyclic groups of 6 to 10 carbon atoms (C₆-C₁₀ arylene). Examples, without limitation, of aryl groups are phenyl, naphthalenyl and anthracenyl. Examples, without limitation, of aryl groups are phenylene, naphthalenylene and anthracenylene. It will be appreciated that an aryl or arylene group can be unsubstituted or substituted as described herein. An aryl or arylene group can be substituted with any of the substituents in the various embodiments described herein, including one or more of such substituents.

The term “heterocycloalkyl” refers to a mono-valent monocyclic or polycyclic ring structure that is saturated or partially saturated having one or more non-carbon ring atoms. The term “heterocycloalkylene” refers to a mono-valent monocyclic or polycyclic ring structure that is saturated or partially saturated having one or more non-carbon ring atoms. In some embodiments, it can be advantageous to limit the number of atoms in a “heterocycloalkyl” or “heterocycloalkylene” to a specific range of ring atoms, such as from 3 to 12 ring atoms (3- to 12-membered), or 3 to 7 ring atoms (3- to 7-membered), or 3 to 6 ring atoms (3- to 6-membered), or 4 to 6 ring atoms (4- to 6-membered), or 5 to 7 ring atoms (5- to 7-membered). In some embodiments, it can be advantageous to limit the number and type of ring heteroatoms in “heterocycloalkyl” or “heterocycloalkylene” to a specific range or type of heteroatoms, such as 1 to 5 ring heteroatoms selected from nitrogen, oxygen, and sulfur. Polycyclic ring systems include fused, bridged, and spiro systems. The ring structure may optionally contain an oxo group on a carbon ring member or up to two oxo groups on sulfur ring members. Illustrative examples of heterocycloalkyl groups include mono-valent radicals of the following entities, while heterocycloalkylene groups include di-valent radicals of the following entities, in the form of properly bonded moieties:

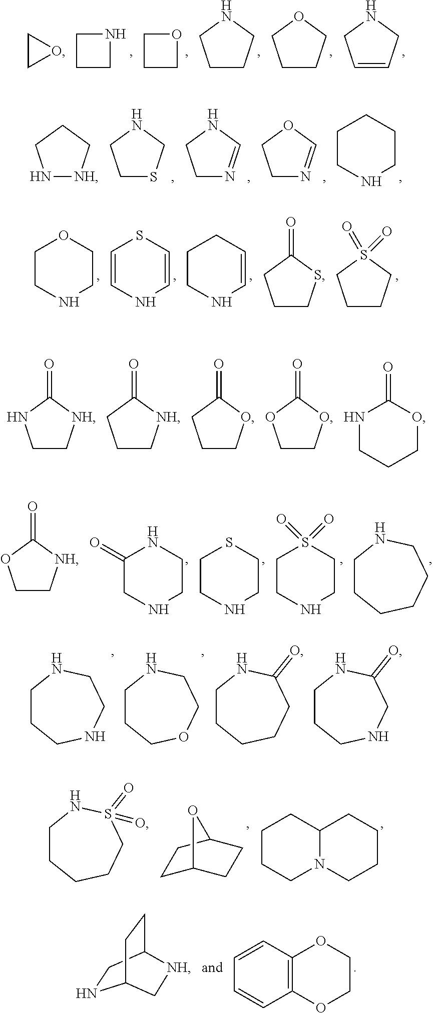

A three-membered heterocycle may contain at least one heteroatom ring atom, where the heteroatom ring atom is a sulfur, oxygen, or nitrogen. Non-limiting examples of three-membered heterocycle groups include monovalent and divalent radicals of oxirane, azetidine, and thiirane. A four-membered heterocycle may contain at least one heteroatom ring atom, where the heteroatom ring atom is a sulfur, oxygen, or nitrogen. Non-limiting examples of four-membered heterocycle groups include monovalent and divalent radicals of azitidine, oxtenane, and thietane. A five-membered heterocycle can contain up to four heteroatom ring atoms, where (a) at least one ring atom is oxygen and sulfur and zero, one, two, or three ring atoms are nitrogen, or (b) zero ring atoms are oxygen or sulfur and up to four ring atoms are nitrogen. Non-limiting examples of five-membered heterocyle groups include mono-valent and divalent radicals of pyrrolidine, tetrahydrofuran, 2,5-dihydro-1H-pyrrole, pyrazolidine, thiazolidine, 4,5-dihydro-1H-imidazole, dihydrothiophen-2(3H)-one, tetrahydrothiophene 1,1-dioxide, imidazolidin-2-one, pyrrolidin-2-one, dihydrofuran-2(3H)-one, 1,3-dioxolan-2-one, and oxazolidin-2-one. A six-membered heterocycle can contain up to four heteroatom ring atoms, where (a) at least one ring atom is oxygen and sulfur and zero, one, two, or three ring atoms are nitrogen, or (b) zero ring atoms are oxygen or sulfur and up to four ring atoms are nitrogen. Non-limiting examples of six-membered heterocycle groups include mono-valent or divalent radicals of piperidine, morpholine, 4H-1,4-thiazine, 1,2,3,4-tetrahydropyridine, piperazine, 1,3-oxazinan-2-one, piperazin-2-one, thiomorpholine, and thiomorpholine 1,1-dioxide. A “heterobicycle” is a fused bicyclic system comprising one heterocycle ring fused to a cycloalkyl or another heterocycle ring.

It will be appreciated that a heterocycloalkyl or heterocycloalkylene group can be unsubstituted or substituted as described herein. A heterocycloalkyl or heterocycloalkylene group can be substituted with any of the substituents in the various embodiments described herein, including one or more of such substituents.

The term “heteroaryl” refers to a mono-valent monocyclic, fused bicyclic, or fused polycyclic aromatic heterocycle (ring structure having ring atoms or members selected from carbon atoms and up to four heteroatoms selected from nitrogen, oxygen, and sulfur) that is fully unsaturated and having from 3 to 12 ring atoms per heterocycle. The term “heteroarylene” refers to a di-valent monocyclic, fused bicyclic, or fused polycyclic aromatic heterocycle (ring structure having ring atoms or members selected from carbon atoms and up to four heteroatoms selected from nitrogen, oxygen, and sulfur) having from 3 to 12 ring atoms per heterocycle. In some embodiments, it can be advantageous to limit the number of ring atoms in a “heteroaryl” or “heteroarylene” to a specific range of atom members, such as 5- to 10-membered heteroaryl or 5- to 10-membered heteroarylene. In some instances, a 5- to 10-membered heteroaryl can be a monocyclic ring or fused bicyclic rings having 5- to 10-ring atoms wherein at least one ring atom is a heteroatom, such as N, O, or S. In some instances, a 5- to 10-membered heteroarylene can be a monocyclic ring or fused bicyclic rings having 5- to 10-ring atoms wherein at least one ring atom is a heteroatom, such as N, O, or S. Illustrative examples of 5- to 10-membered heteroaryl groups include mono-valent radicals of the following entities, while examples of 5- to 10-membered heteroarylene groups include di-valent radicals of the following entities, in the form of properly bonded moieties:

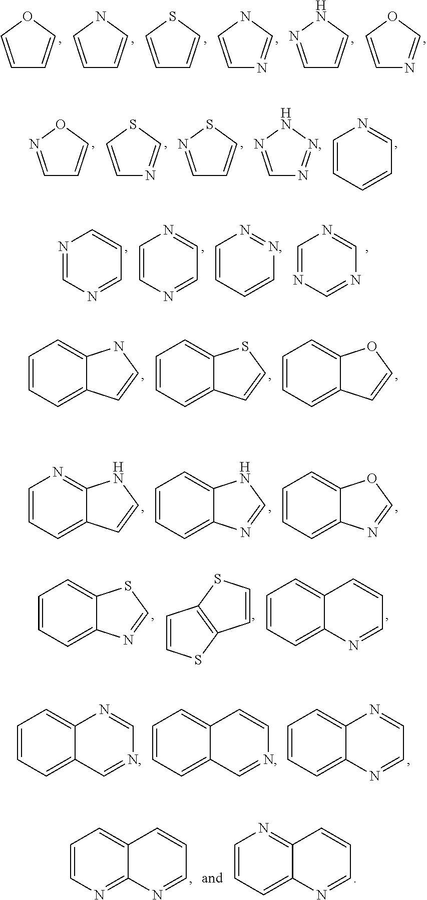

In some embodiments, a “monocyclic” heteroaryl can be an aromatic five- or six-membered heterocycle. A five-membered heteroaryl or heteroarylene can contain up to four heteroatom ring atoms, where (a) at least one ring atom is oxygen and sulfur and zero, one, two, or three ring atoms are nitrogen, or (b) zero ring atoms are oxygen or sulfur and up to four ring atoms are nitrogen. Non-limiting examples of five-membered heteroaryl groups include mono-valent radicals of furan, thiophene, pyrrole, oxazole, isoxazole, thiazole, isothiazole, pyrazole, imidazole, oxadiazole, thiadiazole, triazole, or tetrazole. Non-limiting examples of five-membered heteroarylene groups include di-valent radicals of furan, thiophene, pyrrole, oxazole, isoxazole, thiazole, isothiazole, pyrazole, imidazole, oxadiazole, thiadiazole, triazole, or tetrazole. A six-membered heteroaryl or heteroarylene can contain up to four heteroatom ring atoms, where (a) at least one ring atom is oxygen and sulfur and zero, one, two, or three ring atoms are nitrogen, or (b) zero ring atoms are oxygen or sulfur and up to four ring atoms are nitrogen. Non-limiting examples of six-membered heteroaryl groups include monovalent radicals of pyridine, pyrazine, pyrimidine, pyridazine, or triazine. Non-limiting examples of six-membered heteroarylene groups include divalent radicals of pyridine, pyrazine, pyrimidine, pyridazine, or triazine. A “bicyclic heteroaryl” or “bicyclic heteroarylene” is a fused bicyclic system comprising one heteroaryl ring fused to a phenyl or another heteroaryl ring. Non-limiting examples of bicyclic heteroaryl groups include monovalent radicals of quinoline, isoquinoline, quinazoline, quinoxaline, 1,5-naphthyridine, 1,8-naphthyridine, isoquinolin-3(2H)-one, thieno[3,2-b]thiophene, 1H-pyrrolo[2,3-b]pyridine, 1H-benzo[d]imidazole, benzo[d]oxazole, and benzo[d]thiazole. Non-limiting examples of bicyclic heteroarylene groups include divalent radicals of quinoline, isoquinoline, quinazoline, quinoxaline, 1,5-naphthyridine, 1,8-naphthyridine, isoquinolin-3(2H)-one, thieno[3,2-b]thiophene, 1H-pyrrolo[2,3-b]pyridine, 1H-benzo[d]imidazole, benzo[d]oxazole, and benzo[d]thiazole.

In particular, a pyrrolyl moiety can be depicted by the structural formula

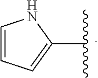 [SMILES: *c1ccc[nH]1]

In particular, a pyrrolylene moiety can be depicted by the structural formula

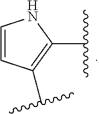 [SMILES: *c1cc[nH]c1*]

It will be appreciated that a heteroaryl or heteroarylene group can be unsubstituted or substituted as described herein. A heteroaryl or heteroarylene group can be substituted with any of the substituents in the various embodiments described herein, including one or more of such substituents.

The term “oxo” represents a carbonyl oxygen. For example, a cyclopentyl substituted with oxo is cyclopentanone.

The term “substituted” means that the specified group or moiety bears one or more substituents. The term “unsubstituted” means that the specified group bears no substituents. Where the term “substituted” is used to describe a structural system, the substitution is meant to occur at any valency-allowed position on the system. In some embodiments, “substituted” means that the specified group or moiety bears one, two, or three substituents. In other embodiments, “substituted” means that the specified group or moiety bears one or two substituents. In still other embodiments, “substituted” means the specified group or moiety bears one substituent.

Any formula depicted herein is intended to represent a compound of that structural formula as well as certain variations or forms. For example, a formula given herein is intended to include a racemic form, or one or more enantiomeric, diastereomeric, or geometric isomers, or a mixture thereof. Additionally, any formula given herein is intended to refer also to a hydrate, solvate, or polymorph of such a compound, or a mixture thereof.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, and ¹²⁵I, respectively. Such isotopically labelled compounds are useful in metabolic studies (preferably with ¹⁴C), reaction kinetic studies (with, for example ²H or 3H), detection or imaging techniques [such as positron emission tomography (PET) or single-photon emission computed tomography (SPECT)] including drug or substrate tissue distribution assays, or in radioactive treatment of patients. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

The nomenclature “(ATOM)ᵢ₋ⱼ” with j>i, when applied herein to a class of substituents, is meant to refer to embodiments of this disclosure for which each and every one of the number of atom members, from i to j including i and j, is independently realized. By way of example, the term C₁₋₃ refers independently to embodiments that have one carbon member (C₁), embodiments that have two carbon members (C₂), and embodiments that have three carbon members (C₃).

Any disubstituent referred to herein is meant to encompass the various attachment possibilities when more than one of such possibilities are allowed. For example, reference to disubstituent -A-B—, where A≠B, refers herein to such disubstituent with A attached to a first substituted member and B attached to a second substituted member, and it also refers to such disubstituent with A attached to the second substituted member and B attached to the first substituted member. For example, in certsain embodiments, where applicable, a compound portion -(L)ₙ-having the formula —CH(CH₃)—CH₂NH—(CH₂)₂—, connecting two groups, A and B, will be understood that —CH(CH₃)—CH₂NH—(CH₂)₂—, can include both of the embodiments A-CH(CH₃)—CH₂NH—(CH₂)₂—B and B—CH(CH₃)—CH₂NH—(CH₂)₂-A. More particularly in the present case, compounds of the formula (I)-(VIII) having a compound portion -(L)ₙ- of the formula —CH(CH₃)—CH₂NH—(CH₂)₂— connecting groups —Z— and —NR²— will be understood to include both embodiments —Z—CH(CH₃)—CH₂NH—(CH₂)₂—NR²— and —NR²—CH(CH₃)—CH₂NH—(CH₂)₂-A.

The disclosure also includes pharmaceutically acceptable salts of the compounds represented by Formula (I)-(VIII), preferably of those described above and of the specific compounds exemplified herein, and pharmaceutical compositions comprising such salts, and methods of using such salts.

A “pharmaceutically acceptable salt” is intended to mean a salt of a free acid or base of a compound represented herein that is non-toxic, biologically tolerable, or otherwise biologically suitable for administration to the subject. See, generally, S. M. Berge, et al., “Pharmaceutical Salts,” J. Pharm. Sci., 1977, 66, 1-19. Preferred pharmaceutically acceptable salts are those that are pharmacologically effective and suitable for contact with the tissues of subjects without undue toxicity, irritation, or allergic response. A compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt.

Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington's Pharmaceutical Sciences, 17th Edition, Mack Publishing Company, Easton, Pa., 1985.

For a compound of Formula (I)-(VIII) that contains abasic nitrogen, a pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, or ethanesulfonic acid, or any compatible mixture of acids such as those given as examples herein, and any other acid and mixture thereof that are regarded as equivalents or acceptable substitutes in light of the ordinary level of skill in this technology.

The disclosure also relates to pharmaceutically acceptable prodrugs of the compounds of Formula (I)-(VIII), and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula (I)-(VIII)). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985.

The present disclosure also relates to pharmaceutically active metabolites of compounds of Formula (I)-(VIII), and uses of such metabolites in the methods of the disclosure. A “pharmaceutically active metabolite” means a pharmacologically active product of metabolism in the body of a compound of Formula (I)-(VIII) or salt thereof. Prodrugs and active metabolites of a compound may be determined using routine techniques known or available in the art. See, e.g., Bertolini et al., J. Med. Chem. 1997, 40, 2011-2016; Shan et al., J. Pharm. Sci. 1997, 86 (7), 765-767; Bagshawe, Drug Dev. Res. 1995, 34, 220-230; Bodor, Adv. Drug Res. 1984, 13, 255-331; Bundgaard, Design of Prodrugs (Elsevier Press, 1985); and Larsen, Design and Application of Prodrugs, Drug Design and Development (Krogsgaard-Larsen et al., eds., Harwood Academic Publishers, 1991).

As used herein, the term “protecting group” or “PG” refers to any group as commonly known to one of ordinary skill in the art that can be introduced into a molecule by chemical modification of a functional group, such as an amine or hydroxyl, to obtain chemoselectivity in a subsequent chemical reaction. It will be appreciated that such protecting groups can be subsequently removed from the functional group at a later point in a synthesis to provide further opportunity for reaction at such functional groups or, in the case of a final product, to unmask such functional group. Protecting groups have been described in, for example, Wuts, P. G. M., Greene, T. W., Greene, T. W., & John Wiley & Sons. (2006). Greene's protective groups in organic synthesis. Hoboken, N. J: Wiley-Interscience. One of skill in the art will readily appreciate the chemical process conditions under which such protecting groups can be installed on a functional group. Suitable amine protecting groups useful in connection with the present disclosure include, but are not limited to, 9-Fluorenylmethyl-carbonyl (FMOC), t-butylcarbonyl (Boc), benzyloxycarbonyl (Cbz), acetyl (Ac), trifluoroacetyl, phthalimide, benzyl (Bn), triphenylmethyl (trityl, Tr), benzylidene, and p-toluenesulfonyl (tosylamide, Ts).

### REPRESENTATIVE EMBODIMENTS

In some embodiments, the disclosure provides a compound of the formula I, or a pharmaceutically acceptable salt thereof,

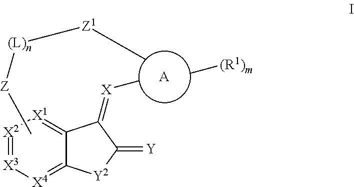

wherein R¹, A, L, X, X¹, X², X³, X⁴, Y, Y², Z, Z¹ m and n are as described herein.

In some embodiments, the disclosure provides a compound of the formula II, or a pharmaceutically acceptable salt thereof,

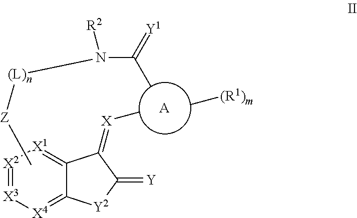

wherein R¹, R², A, L, X, X¹, X², X³, X⁴, Y, Y¹, Y², Z, m and n are as described herein.

In some embodiments, the disclosure provides a compound of the formula III, or a pharmaceutically acceptable salt thereof,

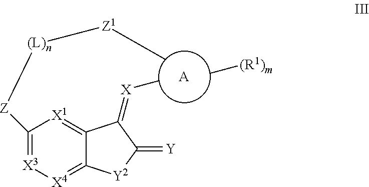

wherein R¹, A, L, X, X¹, X², X³, X⁴, Y, Y², Z, Z¹, m and n are as described herein.

In some embodiments, the disclosure provides a compound of the formula IV, or a pharmaceutically acceptable salt thereof,

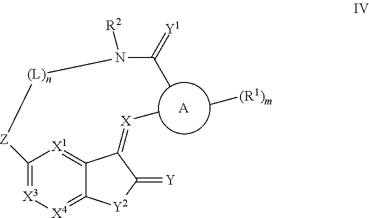

wherein R¹, R², A, L, X, X¹, X², X³, X⁴, Y, Y¹, Y², Z, m and n are as described herein.

In some embodiments, the disclosure provides a compound of the formula V, or a pharmaceutically acceptable salt thereof,

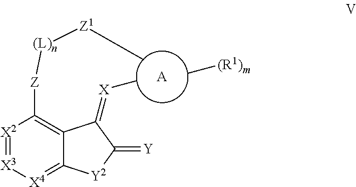

wherein R¹, A, L, X, X¹, X², X³, X⁴, Y, Y¹, Z, Z¹, m and n are as described herein.

In some embodiments, the disclosure provides a compound of the formula VI, or a pharmaceutically acceptable salt thereof,

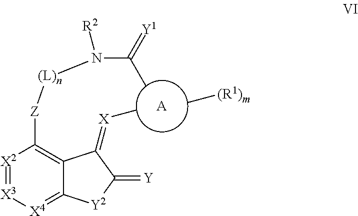

wherein R¹, R², A, L, X, X¹, X², X³, X⁴, Y, Y¹, Y², Z, m and n are as described herein.

In some embodiments, the disclosure provides a compound of the formula VII, or a pharmaceutically acceptable salt thereof,

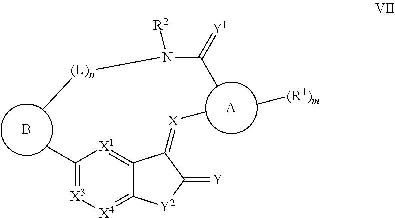

wherein R¹, R², A, B, L, X, X¹, X², X³, X⁴, Y, Y¹, Y², Z, m and n are as described herein.

In some embodiments, the disclosure provides a compound of the formula VIII, or a pharmaceutically acceptable salt thereof,

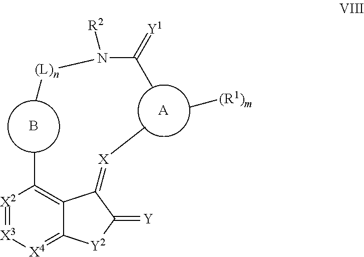

wherein R¹, R², A, B, L, X, X¹, X², X³, X⁴, Y, Y¹, Y², Z, m and n are as described herein.

In some embodiments, Ring A is a 5- to 10-membered heteroarylene and Z is a 3- to 7-membered heterocycloalkylene, C₃-C₆ cycloalkylene, C₆-C₁₀ arylene, or 5- to 10-membered heteroarylene (a.k.a Ring B). In some embodiments, Ring A is a 5- to 10-heteroarylene and Ring B is a 5- to 10-membered heteroarylene. In some embodiments, Ring A is a 5- to 10-heteroarylene and Ring B is a 3- to 7-membered heterocycloalkylene. In some embodiments, Ring A is a 5- to 10-heteroarylene and Ring B is a C₃-C₆ cycloalkylene. In some embodiments, Ring A is a 5- to 10-heteroarylene and Ring B is a C₆-C₁₀ arylene.

In some embodiments, Ring A is a C₆-C₁₀ arylene and Z is a 3- to 7-membered heterocycloalkylene, C₃-C₆ cycloalkylene, C₆-C₁₀ arylene, or 5- to 10-membered heteroarylene (a.k.a. Ring B). In some embodiments, Ring A is a C₆-C₁₀ arylene and Ring B is a 5- to 10-membered heteroarylene. In some embodiments, Ring A is a C₆-C₁₀ arylene and Ring B is a 3- to 7-membered heterocycloalkylene. In some embodiments, Ring A is a C₆-C₁₀ arylene and Ring B is a C₃-C₆ cycloalkylene. In some embodiments, Ring A is a C₆-C₁₀ arylene and Ring B is a C₆-C₁₀ arylene.

In some embodiments, Ring A is a 5- or 6-membered heteroarylene, and Z is a 3- to 7-membered heterocycloalkylene, C₃-C₆ cycloalkylene, C₆-C₁₀ arylene, or 5- to 10-membered heteroarylene (a.k.a Ring B). In some embodiments, Ring A is a 5- or 6-heteroarylene and Ring B is a 5- to 10-membered heteroarylene. In some embodiments, Ring A is a 5- or 6-heteroarylene and Ring B is a 3- to 7-membered heterocycloalkylene. In some embodiments, Ring A is a 5- or 6-heteroarylene and Ring B is a C₃-C₆ cycloalkylene. In some embodiments, Ring A is a 5- or 6-heteroarylene and Ring B is a C₆-C₁₀ arylene.

In some embodiments, Ring A is a 5- or 6-membered heteroarylene 1, 2, or 3 nitrogen ring atoms. In some embodiments, Ring A is furanylene, thiophenylene, pyrrolylene, oxazolylene, isoxazolylene, thiazolylene, isothiazolylene, pyrazolylene, imidazolylene, oxadiazolylene, thiadiazolylene, triazolylene, pyridinylene, pyrazinylene, pyrimidinylene, pyridazinylene, or triazinylene. In some embodiments, Ring A is pyrrolylene. In some embodiments, Ring B is a 5- or 6-membered heteroarylene containing 1 or 2 nitrogen ring atoms. In some embodiments, Ring B is a pyrazolylene, oxazolylene, thiazolylene, pyridinylene, pyrimidinylene, and pyridin-2-onylene. In some embodiments, Ring A is pyrrolylene, and Ring B is a pyrazolylene, oxazolylene, thiazolylene, pyridinylene, pyrimidinylene, and pyridin-2-onylene.

In some embodiments, Ring A is of the formula

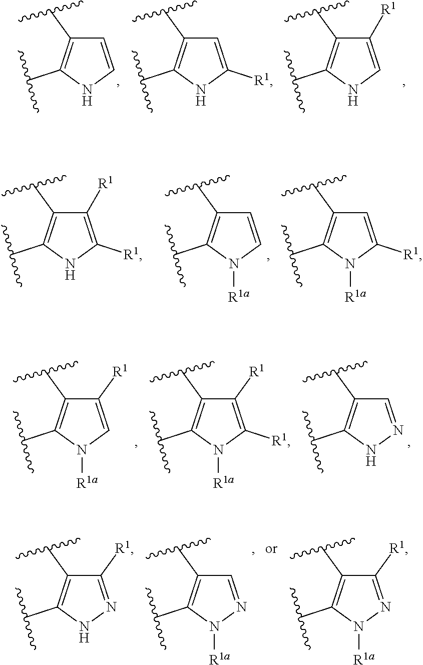

wherein R^{1a} is C₁-C₆ alkyl, —C(O)R^{a}, —C(O)OR^{a}, —C(O)NR^{a}R^{b}, or —P(O)₂OR^{a}, wherein each hydrogen atom in C₁-C₆ alkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, Ring A is of the formula

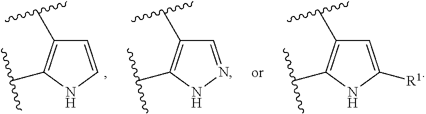

In some embodiments, Ring B (Z) is of the formula

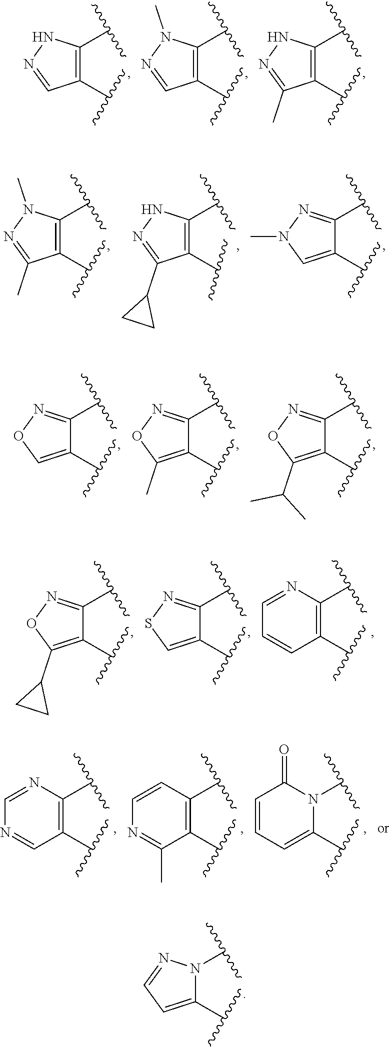

In some embodiments, Ring B (Z) is of the formula

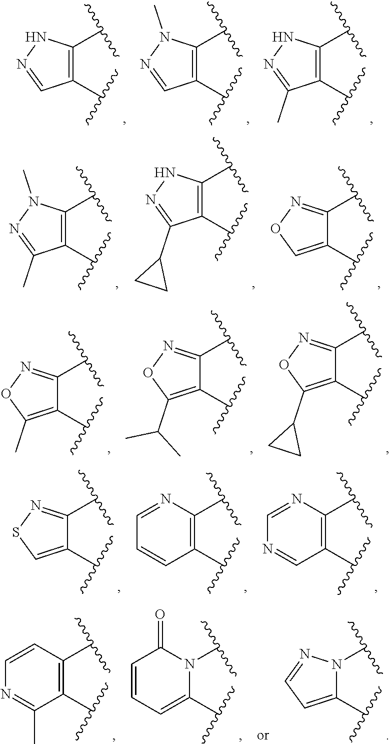

In some embodiments, Ring B (Z) is of the formula

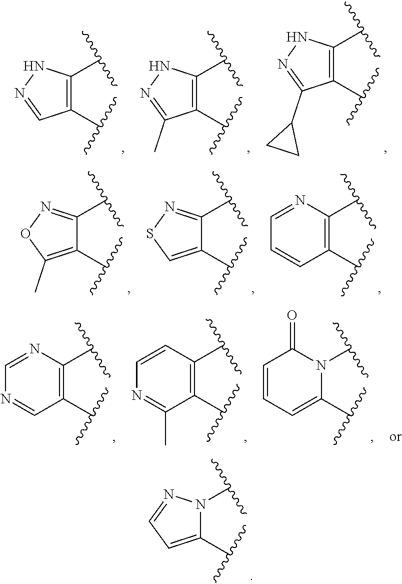

In some embodiments, Ring B (Z) is not

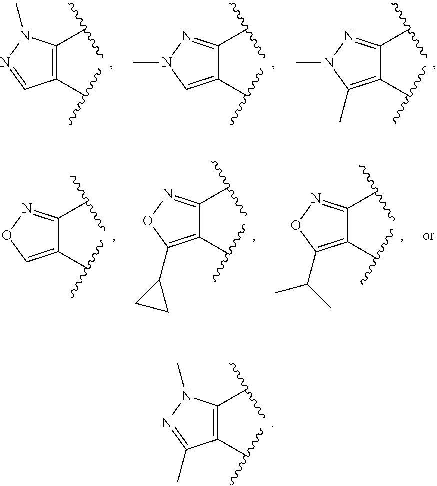

In some embodiments, Ring B (Z) is not

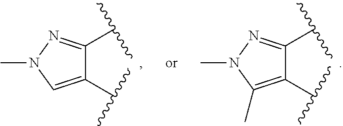

In some embodiments, Ring B (Z) is C₆-C₁₀ arylene, wherein each hydrogen atom in C₆-C₁₀ aryl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, Ring B is phenylene, wherein each hydrogen atom in phenylene is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, Ring B is of the formula

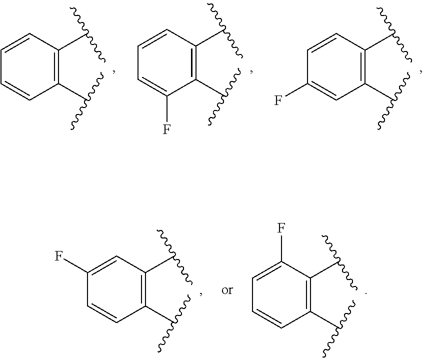 [SMILES: *c1ccccc1* ; *c1cccc(F)c1* ; *c1ccc(F)cc1* ; *c1ccc(F)cc1* ; *c1cccc(F)c1*]

In some embodiments, Ring B (Z) is 3- to 7-membered heterocycloalkylene, wherein each hydrogen atom in 3- to 7-membered heterocycloalkylene is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, Ring B is pyrrolidonylene or azetidinylene, wherein each hydrogen atom in pyrrolidonylene and azetidinylene is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, Ring A is a 5- or 6-membered heteroarylene, and Z is —C(R¹²)(R¹³)—, —O—, —N(R¹⁴)—, —S—, —S(O)— or —S(O)₂—.

In some embodiments, each R¹, when present, is independently deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂

In some embodiments, R¹, when present, is —CN or C₁-C₆ alkyl, wherein each hydrogen atom in C₁-C₆ alkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂. In some embodiments, R¹, when present, is R¹ is —CN or methyl.

In some embodiments, R^{1a}, when present, is C₁-C₆ alkyl, —C(O)R^{a}, —C(O)OR^{a}, —C(O)NR^{a}R^{b}, or —P(O)₂OR^{a}, wherein each hydrogen atom in C₁-C₆ alkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂. In some embodiments, Ria, when present, is methyl.

In some embodiments, R² is independently H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}′—CN, or —NO₂.

In some embodiments, R² is H or C₁-C₆ alkyl, wherein each hydrogen atom in C₁-C₆ alkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}′, —CN, or —NO₂. In some embodiments, R² is H or methyl.

In some embodiments, each L is independently —C(R³)(R⁴)—, —C(O)—, —O—, —N(R⁵)—, —S—, —S(O)— or —S(O)₂—, provided that (L)ₙ does not comprise a —O—O—, a —O—S—, or a —O—N(R⁵)— bond.

In some embodiments, each R³, R⁴, R¹² and R¹³ is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{e}, —S(O)R^{e}, —S(O)₂RC, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, —NO₂, or two of R³, R⁴, R¹², and R¹³ taken together with the carbon or carbons to which they are attached form a C₃-C₆ cycloalkyl or a 4- to 6-membered heterocycloalkyl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, or 4- to 6-membered heterocycloalkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, R¹² and R¹³, when present, are independently selected from the group consisting of H, deuterium, fluoro, chloro, bromo, —OR^{e}, and C₁-C₆ alkyl; or R¹² and R¹³ taken together with the carbon to which they are attached form a C₃-C₆ cycloalkyl or a 4- to 6-membered heterocycloalkyl, wherein each hydrogen atom in C₃-C₆ cycloalkyl or 4- to 6-membered heterocycloalkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, when present, R¹² is H and R¹³ is methyl. In some embodiments, when present, R¹² is methyl and R¹³ is H. In some embodiments, when present, R¹² and R¹³ are H. In some embodiments, when present, R¹² is methyl and R¹³ is —OH. In some embodiments, when present, R¹² is —OH and R¹³ is methyl.

In some embodiments, each L is independently selected from the group consisting of —C(O)—, —O—, —CH₂—, —C(H)(CH₃)—, —C(H)(OH)—, —NH—, and —NCH₃—. In some embodiments, -(L)ₙ- is is —(CH₂)₂—, —(CH₂)₃—, —(CH₂)₄—, —(CH₂)₅—, —(CH₂)₆—, —C(O)NH—(CH₂)₂O(CH₂)₂—, —C(O)N(CH₃)—(CH₂)₂O(CH₂)₂—, —NHC(O)CH₂O(CH₂)₂—, —N(CH₃)—C(O)CH₂O(CH₂)₂—, —CH₂O(CH₂)₂—, —(CH₂)₂O(CH₂)₂—, —(CH₂)₂S(CH₂)₂—, —O(CH₂)₂S(CH₂)₂—, —(CH₂)₂SO₂(CH₂)₂—, —O(CH₂)₂SO₂(CH₂)₂—, —(CH₂)₂SO(CH₂)₂—, —O(CH₂)₂SO(CH₂)₂—, —(CH₂)₂O(C(H)(C(O)N(H) (azetidin-3-yl))-CH₂—, —(CH₂)₂O(C(H)(C(O)N(H)(CH₃))—CH₂—, —(CH₂)₂O(C(H)(C(O)N(CH₃)₂)—CH₂—, —(CH₂)₂O(C(H)(C(O)N(H)(piperidin-4-yl))-CH₂—, —(CH₂)₂O(C(H)(C(O)N(H)(pyrrolidin-3-yl))-CH₂—, —(CH₂)₂O(C(H)(C(O)N(H)(4-methylpiperazin-1-yl))-CH₂—, —(CH₂)₂O(C(H)(C(O)OCH₃)—CH₂—, —(CH₂)₃O(CH₂)₂—, —(CH₂)₂O(CH₂)₃—, —CH₂CH(CH₃)—O(CH₂)₂—, —CH(CH₃)—CH₂O(CH₂)₂—, —O(CH₂)₂—, —O—(CH₂)₃—, —OCH₂O(CH₂)₂—, —O—CH₂CH(OH)CH₂—, —O—(CH₂)₂O(CH₂)₂—, —O—CH₂CH(CH₃)—O(CH₂)₂—, —O—CH(CH₃)—CH₂O(CH₂)₂—, —O—(CH₂)₂NH—(CH₂)₂—, —O—CH₂CH(CH₃)—NH—(CH₂)₂—, —O—CH(CH₃)—CH₂NH—(CH₂)₂—, —CH₂NH—(CH₂)₂—, —(CH₂)₂NH—(CH₂)₂—, —CH₂CH(CH₃)—NH—(CH₂)₂—, —CH(CH₃)—CH₂NH—(CH₂)₂—, —O—(CH₂)₂N(CH₃)—(CH₂)₂—, —O—CH₂CH(CH₃)—N(CH₃)—(CH₂)₂—, —O—CH(CH₃)—CH₂N(CH₃)—(CH₂)₂—, —CH₂N(CH₃)—(CH₂)₂—, —CH₂N(CH₂CH₃)—(CH₂)₂—, —CH₂N(CH(CH₃))—(CH₂)₂—, —(CH₂)₂N(CH₃)—(CH₂)₂—, —CH₂CH(CH₃)—N(CH₃)—(CH₂)₂—, or —O—CH(CH₃)—CH₂N(CH₃)—(CH₂)₂—. In some embodiments, —Z-(L)ₙ—Z¹— does not comprise an —O—O—, a —O—S—, or an —O—N(R^{X})— bond.

In some embodiments, R⁵ is H or C₁-C₆ alkyl, wherein each hydrogen atom in C₁-C₆ alkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}′, —CN, or —NO₂. In some embodiments, R⁵ is H or methyl.

In some embodiments, X is —N—. In some embodiments, X is C(R⁶). In some embodiments, R⁶, when present, is H, deuterium, halogen, C₁-C₆ alkyl, C₂-C₆ alkenyl, C₂-C₆ alkynyl, or —CN. In some embodiments, R⁶, when present, is H.

In some embodiments, X¹ is N or C(R⁷); and X² is N or C(R⁸); provided that one of R⁷ or R⁸ is a bond to Z. In some embodiments, X¹ is N or C(R⁷). In some embodiments, X¹ is N.

In some embodiments, X¹ is C(R⁷). In some embodiments, X² is N- or C(R⁸). In some embodiments, X² is N. In some embodiments, X² is C(R⁸). In some embodiments, X³ is N or C(R⁹). In some embodiments, X³ is N. In some embodiments, X³ is C(R⁹). In some embodiments, X⁴ is N or C(R¹⁰). In some embodiments, X⁴ is N. In some embodiments, X⁴ is C(R¹⁰). In some embodiments, X¹ and X³ are N. In some embodiments, X¹ and X⁴ are N. In some embodiments, X³ and X⁴ are N. In some embodiments, X¹ is C(R⁷), X³ is C(R⁹), and X⁴ is C(R¹⁰). In some embodiments, the compound is not a compound wherein X¹ is C(R⁷), X³ is C(R⁹), and X⁴ is C(R¹⁰), and R¹⁰ is not H. In some embodiments, the compound is not a compound wherein X¹ is C(R⁷), X³ is C(R⁹), and X⁴ is C(R¹⁰), and R⁹ is not H. In some embodiments, the compound is not a compound wherein X¹ is C(R⁷), X³ is C(R⁹), and X⁴ is C(R¹⁰), and R⁹ and R¹⁰ are not H.

In some embodiments, each of R⁷ and R⁸ is independently a bond to Z, H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂; wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; provided that one of R⁷ or R⁸ is a bond to Z;

In some embodiments, each of R⁹ and R¹⁰ is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂; or R⁸ and R⁹ or R⁹ and R¹⁰ taken together with the carbons to which they are attached form a C₄-C₆ cycloalkyl, a 4- to 7-membered heterocycloalkyl, or a C₆-C₁₀ aryl, wherein each hydrogen atom in C₁-C₆ alkyl, C₂-C₆ alkenyl, C₂-C₆ alkynyl, C₃-C₆ cycloalkyl, C₄-C₆ cycloalkyl, 3- to 7-membered heterocycloalkyl, C₆-C₁₀ aryl, 5- to 10-membered heteroaryl, or 4- to 7-membered heterocycloalkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, each of R⁹ and R¹⁰ is not deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂; or R⁸ and R⁹ or R⁹ and R¹⁰ taken together with the carbons to which they are attached form a C₄-C₆ cycloalkyl, a 4- to 7-membered heterocycloalkyl, or a C₆-C₁₀ aryl, wherein each hydrogen atom in C₁-C₆ alkyl, C₂-C₆ alkenyl, C₂-C₆ alkynyl, C₃-C₆ cycloalkyl, C₄-C₆ cycloalkyl, 3- to 7-membered heterocycloalkyl, C₆-C₁₀ aryl, 5- to 10-membered heteroaryl, or 4- to 7-membered heterocycloalkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, C(R⁷) is H, deuterium, fluoro, chloro, —CN, or methyl. In some embodiments, C(R⁸) is H, deuterium, fluoro, chloro, —CN, or methyl. In some embodiments, each C(R⁹) is H, deuterium, fluoro, chloro, —CN, or methyl. In some embodiments, C(R¹⁰) is H, deuterium, fluoro, chloro, —CN, or methyl. In some embodiments, C(R⁹) is H. In some embodiments, C(R⁹) is not —Cl. In some embodiments, C(R¹⁰) is H. In some embodiments, C(R¹⁰) is not —Cl.

In some embodiments, the compound is not a compound wherein Ring B (Z) is

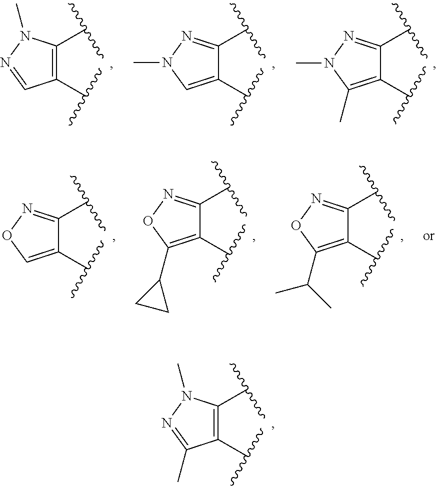

and R⁹ and/or R¹⁰ is not H. In some embodiments, the compound is not a compound wherein Ring B (Z) is

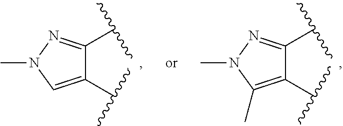

and R⁹ and/or R¹⁰ is not H. In some embodiments, the compound is not a compound wherein X¹ is C(R⁷), X³ is C(R⁹), X⁴ is C(R¹⁰), R⁹ and/or R¹⁰ is not H, and Ring B (Z) is

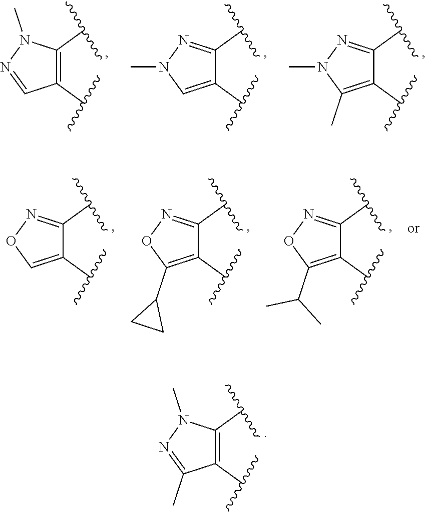

In some embodiments, the compound is not a compound wherein X¹ is C(R⁷), X³ is C(R⁹), X⁴ is C(R¹⁰), R⁹ and/or R¹⁰ is not H, and Ring B (Z) is

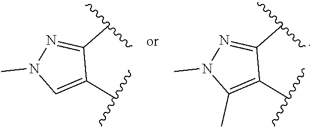

In some embodiments, the compound is not a compound wherein X¹ is C(R⁷), X³ is C(R⁹), X⁴ is C(R¹⁰), R⁹ and/or R¹⁰ is —Cl, and Ring B (Z) is

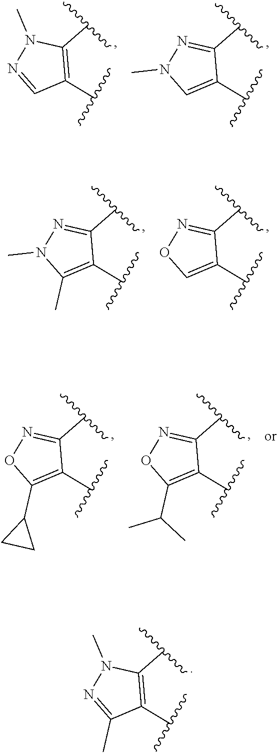

In some embodiments, the compound is not a compound wherein X¹ is C(R⁷), X³ is C(R⁹), X⁴ is C(R¹⁰), R⁹ and/or R¹⁰ is —Cl, and Ring B (Z) is

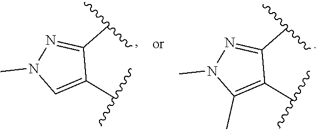

In some embodiments, X¹ is C(R⁷), X³ is C(R⁹), X⁴ is C(R¹⁰), and R⁹ and/or R⁰ is not —Cl. In some embodiments, X¹ is C(R⁷), X³ is C(R⁹), X⁴ is C(R¹⁰), R⁹ and/or R¹⁰ is not

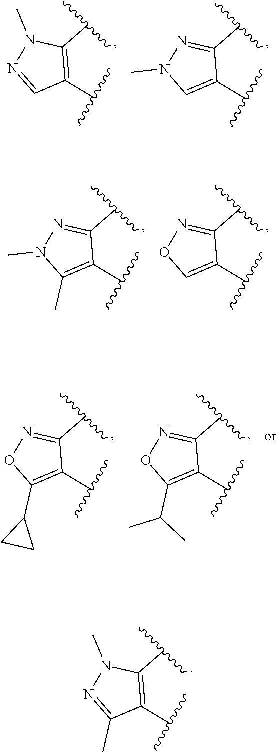

In some embodiments, X¹ is C(R⁷), X³ is C(R⁹), X⁴ is C(R¹⁰), R⁹ and/or R¹⁰ is not —Cl, and Ring B (Z) is not

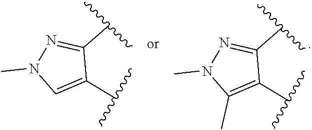

In some embodiments, 0, 1, 2, 3, or 4. In some embodiments, m is 0. In some embodiments, m is 1. In some embodiments, m is 2. In some embodiments, m is 3. In some embodiments, m is 4.

In some embodiments, n is 2, 3, 4, 5, 6, 7, or 8. In some embodiments, n is 2. In some embodiments, n is 3. In some embodiments, n is 4. In some embodiments, n is 5. In some embodiments, n is 6. In some embodiments, n is 7. In some embodiments, n is 8.

In some embodiments, the disclosure provides a compound selected from the group consisting of [3a(4)Z]-10,11-dihydro-2H,13H-16,1-(azenometheno)pyrazolo[4,3-m]dipyrrolo[3,2-f:3′,4′-i][1,4,11]oxadiazacyclotetradecine-3,8(5H,9H)-dione;
[3a(4)Z]-9,10,11,12-tetrahydro-14H-17,1-(azenometheno)pyrazolo[3,4-b]dipyrrolo[3,4-f:2′,3′-i][1,5,12]oxadiazacyclopentadecine-3,8(2H,5H)-dione;
[3a(4)Z]-9,10,11,12-tetrahydro-14H-1,17-(azenometheno)pyrazolo[3,4-b]dipyrrolo[3,4-f:2′,3′-i][1,5,12]oxadiazacyclopentadecine-3,8(2H,5H)-dione;
[3a(4)Z]-9,10,11,12-tetrahydro-14H-1,17-(diazanediylidene)pyrazolo[4,3-n]dipyrrolo[3,2-g:3′,4′-j][1,5]oxazacyclopentadecine-3,8(2H,5H)-dione;
[3a(4)Z]-9,10,11,12-tetrahydro-14H-1,17-(ethanediylidene)pyrazolo[3,4-b]dipyrrolo[3,4-f:2′,3′-i][1,5,12]oxadiazacyclopentadecine-3,8(2H,5H)-dione;
[3a(4)Z]-9,10,11,12-tetrahydro-14H-17,1-(azenometheno)pyrazolo[4,3-n]dipyrrolo[3,2-g:3′,4′-j][1,5]oxazacyclopentadecine-3,8(2H,5H)-dione;
[3a(4)Z]-9,10,11,12-tetrahydro-14H-1,17-(azenometheno)pyrazolo[4,3-n]dipyrrolo[3,2-g:3′,4′-j][1,5]oxazacyclopentadecine-3,8(2H,5H)-dione;
[3a(4)Z]-9,10,11,12-tetrahydro-14H-1,17-(ethanediylidene)pyrazolo[4,3-n]dipyrrolo[3,2-g:3′,4′-j][1,5]oxazacyclopentadecine-3,8(2H,5H)-dione;
[3a(4)Z,11S]-11-hydroxy-9,10,11,12-tetrahydro-14H-1,17-(ethanediylidene)pyrazolo[3,4-b]dipyrrolo[3,4-f:2′,3′-i][1,5,12]oxadiazacyclopentadecine-3,8(2H,5H)-dione;
[19a(20)Z]-2,5-dimethyl-6,7,9,10-tetrahydro-1H,12H-15,17-(ethanediylidene)pyrazolo[4,3-p]dipyrrolo[3,2-i:3′,4′-l][1,4,7,14]dioxadiazacycloheptadecine-4,19(5H,18H)-dione;
[3a(4)Z]-6-methyl-9,10,11,12-tetrahydro-14H-1,17-(ethanediylidene)pyrazolo[3,4-b]dipyrrolo[3,4-f:2′,3′-i][1,5,12]oxadiazacyclopentadecine-3,8(2H,5H)-dione;
[3a(4)Z]-3,8-dioxo-2,3,5,8,9,10,11,12-octahydro-14H-1,17-(ethanediylidene)pyrazolo[3,4-b]dipyrrolo[3,4-f:2′,3′-i][1,5,12]oxadiazacyclopentadecine-6-carbonitrile;
[3a(4)Z]-6-methyl-9,10,11,12-tetrahydro-14H-1,17-(ethanediylidene)imidazo[4,5-i]pyrazolo[3,4-b]pyrrolo[3,4-f][1,5,12]oxadiazacyclopentadecine-3,8(2H,5H)-dione;
[3a(4)Z]-6,15-dimethyl-9,10,11,12-tetrahydro-15H-1,17-(ethanediylidene)pyrazolo[3,4-b]dipyrrolo[3,4-f:2′,3′-i][1,5,12]oxadiazacyclopentadecine-3,8(2H,5H)-dione;
[3a(4)Z]-6-methyl-9,10,11,12-tetrahydro-1,17-(ethanediylidene)[1,2]oxazolo[3,4-b]dipyrrolo[3,4-f:2′,3′-i][1,5,12]oxadiazacyclopentadecine-3,8(2H,5H)-dione;
[3a(4)Z]-6,16-dimethyl-9,10,11,12-tetrahydro-1,17-(ethanediylidene)[1,2]oxazolo[3,4-b]dipyrrolo[3,4-f:2′,3′-i][1,5,12]oxadiazacyclopentadecine-3,8(2H,5H)-dione;
[3a(4)Z]-6-methyl-9,10,11,12-tetrahydro-1,17-(ethanediylidene)dipyrrolo[3,4-f:2′,3′-i][1,2]thiazolo[3,4-b][1,5,12]oxadiazacyclopentadecine-3,8(2H,5H)-dione;
[3a(4)Z]-6,9-dimethyl-9,10,11,12-tetrahydro-14H-1,17-(ethanediylidene)pyrazolo[3,4-b]dipyrrolo[3,4-f:2′,3′-i][1,5,12]oxadiazacyclopentadecine-3,8(2H,5H)-dione;
[3a(4)Z]-6,9-dimethyl-9,10,11,12-tetrahydro-1,17-(ethanediylidene)[1,2]oxazolo[3,4-b]dipyrrolo[3,4-f:2′,3′-i][1,5,12]oxadiazacyclopentadecine-3,8(2H,5H)-dione;
[3a(4)Z]-6,9,16-trimethyl-9,10,11,12-tetrahydro-14H-1,17-(ethanediylidene)pyrazolo[3,4-b]dipyrrolo[3,4-f:2′,3′-i][1,5,12]oxadiazacyclopentadecine-3,8(2H,5H)-dione;
[3a(4)Z]-6,9,16-trimethyl-9,10,11,12-tetrahydro-1,17-(ethanediylidene)[1,2]oxazolo[3,4-b]dipyrrolo[3,4-f:2′,3′-i][1,5,12]oxadiazacyclopentadecine-3,8(2H,5H)-dione;
[3a(4)Z]-6-methyl-9,10,11,12-tetrahydro-1,17-(ethanediylidene)pyrazolo[5,1-c]dipyrrolo[3,2-j:3′,4′-m][1,4,8]triazacyclotetradecine-3,8(2H,5H)-dione;
[3a(4)Z]-6-methyl-9,10,11,12-tetrahydro-17,1-(azenometheno)pyrazolo[1,5-e]dipyrrolo[3,4-i:2′,3′-l][1,5]diazacyclotetradecine-3,8(2H,5H)-dione;
[19a(20)Z]-2-methyl-6,7,9,10-tetrahydro-1H-15,17-(ethanediylidene)pyrazolo[1,5-d]dipyrrolo[3,4-h:2′,3′-k][1,4,7,14]oxatriazacyclohexadecine-4,19(5H,18H)-dione;
[19a(20)Z]-2-methyl-6,7,9,10-tetrahydro-1H-15,17-(azenometheno)pyrazolo[1,5-d]dipyrrolo[3,4-h:2′,3′-k][1,4,14]oxadiazacyclohexadecine-4,19(5H,18H)-dione;
[19a(20)Z]-2-methyl-6,7,9,10-tetrahydro-1H-15,17-(azenometheno)pyrazolo[1,5-d]dipyrrolo[3,4-h:2′,3′-k][1,4,7,14]oxatriazacyclohexadecine-4,19(5H,18H)-dione;
[19a(20)Z]-2-methyl-6,7,9,10-tetrahydro-1H-15,17-(ethanediylidene)pyrazolo[1,5-d]dipyrrolo[3,4-h:2′,3′-k][1,4,14]oxadiazacyclohexadecine-4,19(5H,18H)-dione;

[I1R,19a(20)Z]-2,10-dimethyl-6,7,9,10-tetrahydro-1H-15,17-(ethanediylidene)pyrazolo[1,5-d]dipyrrolo[3,4-h:2′,3′-k][1,4,14]oxadiazacyclohexadecine-4,19(5H,18H)-dione;
[19a(20)Z]-2,5-dimethyl-6,7,9,10-tetrahydro-1H-15,17-(ethanediylidene)pyrazolo[1,5-d]dipyrrolo[3,4-h:2′,3′-k][1,4,14]oxadiazacyclohexadecine-4,19(5H,18H)-dione;
[19a(20)Z]-2,5-dimethyl-6,7,9,10-tetrahydro-1H-15,17-(azenometheno)pyrazolo[1,5-d]dipyrrolo[3,4-h:2′,3′-k][1,4,7,14]oxatriazacyclohexadecine-4,19(5H,18H)-dione;
[19a(20)Z]-2-methyl-5,6,7,8,9,10-hexahydro-15,17-(ethanediylidene)pyrazolo[1,5-g]dipyrrolo[3,4-k:2′,3′-n][1,4,7]triazacyclohexadecine-4,19(1H,18H)-dione;
[19a(20)Z]-2,5-dimethyl-5,6,7,8,9,10-hexahydro-15,17-(ethanediylidene)pyrazolo[1,5-g]dipyrrolo[3,4-k:2′,3′-n][1,4,7]triazacyclohexadecine-4,19(1H,18H)-dione;
[3a(4)Z]-6-methyl-10,11,13,14-tetrahydro-2H-1,17-(ethanediylidene)pyrazolo[4,3-m]dipyrrolo[3,2-f:3′,4′-i][1,4,11]oxadiazacyclopentadecine-3,8(5H,9H)-dione;
[3a(4)Z]-6-methyl-10,11,13,14-tetrahydro-2H-17,1-(azenometheno)pyrazolo[4,3-m]dipyrrolo[3,2-f:3′,4′-i][1,4]oxazacyclopentadecine-3,8(5H,9H)-dione;
[3a(4)Z]-6-methyl-10,11,13,14-tetrahydro-2H-1,17-(ethanediylidene)pyrazolo[4,3-m]dipyrrolo[3,2-f:3′,4′-i][1,4]oxazacyclopentadecine-3,8(5H,9H)-dione;
[3a(4)Z]-6,9-dimethyl-10,11,13,14-tetrahydro-2H-1,17-(ethanediylidene)pyrazolo[4,3-m]dipyrrolo[3,2-f:3′,4′-i][1,4]oxazacyclopentadecine-3,8(5H,9H)-dione;
[3a(4)Z]-6,9,16-trimethyl-10,11,13,14-tetrahydro-2H-1,17-(ethanediylidene)pyrazolo[4,3-m]dipyrrolo[3,2-f:3′,4′-i][1,4]oxazacyclopentadecine-3,8(5H,9H)-dione;
[3a(4)Z]-6-methyl-9,10,11,12,13,14-hexahydro-17,1-(azenometheno)pyrazolo[3,4-f]dipyrrolo[3,4-j:2′,3′-m][1,4,9]triazacyclopentadecine-3,8(2H,5H)-dione;
[3a(4)Z]-6-methyl-9,10,11,12,13,14-hexahydro-1,17-(ethanediylidene)pyrazolo[3,4-f]dipyrrolo[3,4-j:2′,3′-m][1,4,9]triazacyclopentadecine-3,8(2H,5H)-dione;
[3a(4)Z]-6-methyl-9,10,11,12,13,14-hexahydro-17,1-(azenometheno)pyrazolo[4,3-m]dipyrrolo[3,2-f:3′,4′-i][1,4]diazacyclopentadecine-3,8(2H,5H)-dione;
[3a(4)Z]-6,9-dimethyl-9,10,11,12,13,14-hexahydro-17,1-(azenometheno)pyrazolo[3,4-f]dipyrrolo[3,4-j:2′,3′-m][1,4,9]triazacyclopentadecine-3,8(2H,5H)-dione;
[3a(4)Z]-6,9-dimethyl-9,10,11,12,13,14-hexahydro-1,17-(ethanediylidene)pyrazolo[3,4-f]dipyrrolo[3,4-j:2′,3′-m][1,4,9]triazacyclopentadecine-3,8(2H,5H)-dione;
[3a(4)Z]-6,9-dimethyl-9,10,11,12,13,14-hexahydro-17,1-(azenometheno)pyrazolo[4,3-m]dipyrrolo[3,2-f:3′,4′-i][1,4]diazacyclopentadecine-3,8(2H,5H)-dione;
[3a(4)Z]-6,9-dimethyl-9,10,11,12,13,14-hexahydro-1,17-(ethanediylidene)pyrazolo[4,3-m]dipyrrolo[3,2-f:3′,4′-i][1,4]diazacyclopentadecine-3,8(2H,5H)-dione;
[3a(4)Z]-20-fluoro-6,9-dimethyl-9,10,11,12,13,14-hexahydro-1,17-(ethanediylidene)pyrazolo[4,3-m]dipyrrolo[3,2-f:3′,4′-i][1,4]diazacyclopentadecine-3,8(2H,5H)-dione;
[3a(4)Z]-19-fluoro-6,9-dimethyl-9,10,11,12,13,14-hexahydro-1,17-(ethanediylidene)pyrazolo[4,3-m]dipyrrolo[3,2-f:3′,4′-i][1,4]diazacyclopentadecine-3,8(2H,5H)-dione;
[3a(4)Z]-6,9,20-trimethyl-9,10,11,12,13,14-hexahydro-17,1-(azenometheno)pyrazolo[4,3-m]dipyrrolo[3,2-f:3′,4′-i][1,4]diazacyclopentadecine-3,8(2H,5H)-dione;
[3a(4)Z]-9,20-dimethyl-9,10,11,12,13,14-hexahydro-1,17-(ethanediylidene)pyrazolo[3,4-f]dipyrrolo[3,4-j:2′,3′-m][1,4,9]triazacyclopentadecine-3,8(2H,5H)-dione;
[3a(4)Z]-6,16-dimethyl-9,10,11,12,13,14-hexahydro-17,1-(azenometheno)pyrazolo[4,3-m]dipyrrolo[3,2 f:3′,4′-i][1,4]diazacyclopentadecine-3,8(2H,5H)-dione;
[3a(4)Z]-6,9,16-trimethyl-9,10,11,12,13,14-hexahydro-17,1-(azenometheno)pyrazolo[4,3-m]dipyrrolo[3,2 f:3′,4′-i][1,4]diazacyclopentadecine-3,8(2H,5H)-dione;
[3a(4)Z]-16-cyclopropyl-6,9-dimethyl-9,10,11,12,13,14-hexahydro-17,1-(azenometheno)pyrazolo[4,3-m]dipyrrolo[3,2 f:3′,4′-i][1,4]diazacyclopentadecine-3,8(2H,5H)-dione;
[3a(4)Z]-16-cyclopropyl-6,9-dimethyl-9,10,11,12,13,14-hexahydro-1,17-(ethanediylidene)pyrazolo[3,4-f]dipyrrolo[3,4-j:2′,3′-m][1,4,9]triazacyclopentadecine-3,8(2H,5H)-dione;
[3a(4)Z]-6,9,16-trimethyl-10,11,12,13-tetrahydro-2H-17,1-(azenometheno)[1,2]oxazolo[4,5-m]dipyrrolo[3,2-f:3′,4′-i][1,4]diazacyclopentadecine-3,8(5H,9H)-dione;
[3a(4)Z]-6,9,16-trimethyl-10,11,12,13-tetrahydro-2H-17,1-(azenometheno)[1,2]oxazolo[4,3-m]dipyrrolo[3,2-f:3′,4′-i][1,4]diazacyclopentadecine-3,8(5H,9H)-dione;
[3a(4)Z]-6,14-dimethyl-10,11,13,14-tetrahydro-2H-17,1-(azenometheno)pyrazolo[4,3-m]dipyrrolo[3,2-f:3′,4′-i][1,4]oxazacyclopentadecine-3,8(5H,9H)-dione;
[3a(4)Z]-6,9,14-trimethyl-10,11,13,14-tetrahydro-2H-1,17-(ethanediylidene)pyrazolo[4,3-m]dipyrrolo[3,2-f:3′,4′-i][1,4]oxazacyclopentadecine-3,8(5H,9H)-dione;
[3a(4)Z]-6,9,14-trimethyl-9,10,11,12,13,14-hexahydro-1,17-(ethanediylidene)pyrazolo[4,3-m]dipyrrolo[3,2-f:3′,4′-i][1,4]diazacyclopentadecine-3,8(2H,5H)-dione;
[3a(4)Z]-6,9,12,14-tetramethyl-9,10,11,12,13,14-hexahydro-1,17-(ethanediylidene)pyrazolo[4,3-m]dipyrrolo[3,2-f:3′,4′-i][1,4]diazacyclopentadecine-3,8(2H,5H)-dione;
[3a(4)Z]-6,9,16-trimethyl-9,10,11,12,13,14-hexahydro-1,17-(ethanediylidene)pyrazolo[4,3-m]dipyrrolo[3,2-f:3′,4′-i][1,4]diazacyclopentadecine-3,8(2H,5H)-dione;
[3a(4)Z]-6,9,16-trimethyl-10,11-dihydro-2H,13H-1,17-(ethanediylidene)[1,2]oxazolo[4,3-m]dipyrrolo[3,2 f:3′,4′-i][1,4]oxazacyclopentadecine-3,8(5H,9H)-dione;
[3a(4)Z]-6,9,16-trimethyl-10,11,12,13-tetrahydro-2H-1,17-(ethanediylidene)[1,2]oxazolo[4,3-m]dipyrrolo[3,2 f:3′,4′-i][1,4]diazacyclopentadecine-3,8(5H,9H)-dione;
[3a(4)Z]-6,9,12,14,16-pentamethyl-9,10,11,12,13,14-hexahydro-1,17-(ethanediylidene)pyrazolo[4,3-m]dipyrrolo[3,2 f:3′,4′-i][1,4]diazacyclopentadecine-3,8(2H,5H)-dione;
[3a(4)Z]-6,9,14,16-tetramethyl-9,10,11,12-tetrahydro-14H-1,17-(ethanediylidene)pyrazolo[4,3-n]dipyrrolo[3,2-g:3′,4′-j][1,5]oxazacyclopentadecine-3,8(2H,5H)-dione;
[3a(4)Z]-6,9,14,16-tetramethyl-10,11,13,14-tetrahydro-2H-1,17-(ethanediylidene)pyrazolo[4,3-m]dipyrrolo[3,2-f:3′,4′-i][1,4]oxazacyclopentadecine-3,8(5H,9H)-dione;
[3a(4)Z]-9,14,16-trimethyl-9,10,11,12-tetrahydro-14H-1,17-(ethanediylidene)pyrazolo[4,3-n]dipyrrolo[3,2-g:3′,4′-j][1,5]oxazacyclopentadecine-3,8(2H,5H)-dione;
[3a(4)Z]-9,14,16-trimethyl-10,11,13,14-tetrahydro-2H-1,17-(ethanediylidene)pyrazolo[4,3-m]dipyrrolo[3,2-f:3′,4′-i][1,4]oxazacyclopentadecine-3,8(5H,9H)-dione;
[3a(4)Z]-12-ethyl-6,9,14-trimethyl-9,10,11,12,13,14-hexahydro-1,17-(ethanediylidene)pyrazolo[4,3-m]dipyrrolo[3,2-f:3′,4′-i][1,4]diazacyclopentadecine-3,8(2H,5H)-dione;
[3a(4)Z]-6,9,14-trimethyl-12-(propan-2-yl)-9,10,11,12,13,14-hexahydro-1,17-(ethanediylidene)pyrazolo[4,3-m]dipyrrolo[3,2-f:3′,4′-i][1,4]diazacyclopentadecine-3,8(2H,5H)-dione;
[3a(4)Z]-16-cyclopropyl-6,9-dimethyl-10,11-dihydro-2H,13H-1,17-(ethanediylidene)[1,2]oxazolo[4,3-m]dipyrrolo[3,2-f:3′,4′-i][1,4]oxazacyclopentadecine-3,8(5H,9H)-dione;
[3a(4)Z]-9,14-dimethyl-9,10,11,12,13,14-hexahydro-1,17-(ethanediylidene)pyrazolo[4,3-m]dipyrrolo[3,2-f:3′,4′-i][1,4]diazacyclopentadecine-3,8(2H,5H)-dione;
[3a(4)Z]-6,9-dimethyl-16-(propan-2-yl)-10,11-dihydro-2H,13H-1,17-(ethanediylidene)[1,2]oxazolo[4,3-m]dipyrrolo[3,2-f:3′,4′-i][1,4]oxazacyclopentadecine-3,8(5H,9H)-dione;
[3a(4)Z]-9-methyl-16-(propan-2-yl)-10,11-dihydro-2H,13H-1,17-(ethanediylidene)[1,2]oxazolo[4,3-m]dipyrrolo[3,2-f:3′,4′-i][1,4]oxazacyclopentadecine-3,8(5H,9H)-dione;
[3a(4)Z]-6,9,14-trimethyl-9,10,11,12-tetrahydro-14H-1,17-(ethanediylidene)pyrazolo[4,3-n]dipyrrolo[3,2-g:3′,4′-j][1,5]oxazacyclopentadecine-3,8(2H,5H)-dione;
[3a(4)Z]-9,14-dimethyl-9,10,11,12-tetrahydro-14H-1,17-(ethanediylidene)pyrazolo[4,3-n]dipyrrolo[3,2-g:3′,4′-j][1,5]oxazacyclopentadecine-3,8(2H,5H)-dione;
[3a(4)Z]-6,9,12,14-tetramethyl-9,10,11,12,13,14-hexahydro-17,1-(azenometheno)pyrazolo[4,3-m]dipyrrolo[3,2-f:3′,4′-i][1,4]diazacyclopentadecine-3,8(2H,5H)-dione;
[3a(4)Z]-9,12,14-trimethyl-9,10,11,12,13,14-hexahydro-17,1-(azenometheno)pyrazolo[4,3-m]dipyrrolo[3,2-f:3′,4′-i][1,4]diazacyclopentadecine-3,8(2H,5H)-dione;
[3a(4)Z]-6,9,12,14-tetramethyl-9,10,11,12,13,14-hexahydro-1,17-(ethanediylidene)pyrazolo[3,4-f]dipyrrolo[3,4-j:2′,3′-m][1,4,9]triazacyclopentadecine-3,8(2H,5H)-dione; and
[3a(4)Z]-9,12,14-trimethyl-9,10,11,12,13,14-hexahydro-1,17-(ethanediylidene)pyrazolo[3,4-f]dipyrrolo[3,4-j:2′,3′-m][1,4,9]triazacyclopentadecine-3,8(2H,5H)-dione;
   or a pharmaceutically acceptable salt thereof.

In other embodiments, the disclosure provides a compound selected from the group consisting of [3a(4)Z]-6-methyl-9,10,11,12-tetrahydro-1,18-(ethanediylidene)dipyrrolo[3,2-g:3′,4′-j][1,5,12]benzoxadiazacyclopentadecine-3,8(2H,5H)-dione;
[3a(4)Z]-6-methyl-10,11-dihydro-2H-1,17-(ethanediylidene)dipyrrolo[3,2-f:3′,4′-i][1,4,11]benzoxadiazacyclotetradecine-3,8(5H,9H)-dione;
[3a(4)Z]-6-methyl-10,11-dihydro-2H-17,1-(azenometheno)dipyrrolo[3,2-f:3′,4′-i][1,4]benzoxazacyclotetradecine-3,8(5H,9H)-dione;
[3a(4)Z]-16-fluoro-6-methyl-10,11-dihydro-2H-1,17-(ethanediylidene)dipyrrolo[3,2-f:3′,4′-i][1,4,11]benzoxadiazacyclotetradecine-3,8(5H,9H)-dione;
[3a(4)Z]-15-fluoro-6-methyl-10,11-dihydro-2H-1,17-(ethanediylidene)dipyrrolo[3,2-f:3′,4′-i][1,4,11]benzoxadiazacyclotetradecine-3,8(5H,9H)-dione;
[3a(4)Z]-14-fluoro-6-methyl-10,11-dihydro-2H-1,17-(ethanediylidene)dipyrrolo[3,2-f:3′,4′-i][1,4,11]benzoxadiazacyclotetradecine-3,8(5H,9H)-dione;
[3a(4)Z]-13-fluoro-6-methyl-10,11-dihydro-2H-1,17-(ethanediylidene)dipyrrolo[3,2-f:3′,4′-i][1,4,11]benzoxadiazacyclotetradecine-3,8(5H,9H)-dione; and
[3a(4)Z]-6,9,12-trimethyl-10,11,12,13-tetrahydro-2H-1,18-(ethanediylidene)dipyrrolo[3,2-g:3′,4′-j][2,5]benzodiazacyclopentadecine-3,8(5H,9H)-dione or a pharmaceutically acceptable salt thereof.

In other embodiments, the disclosure provides a compound selected from the group consisting of [3a(4)Z]-6-methyl-10,11-dihydro-2H-1,17-(ethanediylidene)pyrido[3,2-m]dipyrrolo[3,2-f:3′,4′-i][1,4,11]oxadiazacyclotetradecine-3,8(5H,9H)-dione;
[3a(4)Z]-6-methyl-10,11-dihydro-2H-1,17-(ethanediylidene)pyrimido[5,4-m]dipyrrolo[3,2-f:3′,4′-i][1,4,11]oxadiazacyclotetradecine-3,8(5H,9H)-dione;
[3a(4)Z]-6,16-dimethyl-10,11-dihydro-2H-1,17-(ethanediylidene)pyrido[3,4-m]dipyrrolo[3,2-f:3′,4′-i][1,4,11]oxadiazacyclotetradecine-3,8(5H,9H)-dione;
[3a(4)Z]-6-methyl-9,10,11,12-tetrahydro-14H-1,18-(ethanediylidene)pyrido[2,1-c]dipyrrolo[3,2-j:3′,4′-m][1,4,8]triazacyclotetradecine-3,8,14(2H,5H)-trione;
[3a(4)Z]-6-methyl-9,10,11,12-tetrahydro-14H-18,1-(azenometheno)pyrido[1,2-e]dipyrrolo[3,4-i:2′,3′-l][1,5]diazacyclotetradecine-3,8,14(2H,5H)-trione;
   or a pharmaceutically acceptable salt thereof.

In other embodiments, the disclosure provides a compound selected from the group consisting of [3a(4)Z,13aR]-6-methyl-10,11,12,13,13a,14,15,16-octahydro-2H-18,1-(azenometheno)tripyrrolo[1,2-a:3′,2′-i:3″,4″-l][1,4,7]triazacyclopentadecine-3,8(5H,9H)-dione;
[3a(4)Z,13aR]-6-methyl-9,10,11,12,13,13a,14,15-octahydro-17,1-(azenometheno)azeto[1,2-a]dipyrrolo[3,2-i:3′,4′-l][1,4,7]triazacyclopentadecine-3,8(2H,5H)-dione;
[16a(17)Z]-2,11-dimethyl-6,7,10,11-tetrahydro-1H,9H-12,14-(azenometheno)dipyrrolo[3,4-g:2′,3′-j][1,4,6,13]oxatriazacyclopentadecine-4,16(5H,15H)-dione;
[16a(17)Z]-2,5,11-trimethyl-6,7,10,11-tetrahydro-1H,9H-12,14-(azenometheno)dipyrrolo[3,2-f:3′,4′-i][1,4,13]oxadiazacyclopentadecine-4,16(5H,15H)-dione;
[17a(18)Z]-2,12-dimethyl-6,7,9,10,11,12-hexahydro-1H-13,15-(azenometheno)dipyrrolo[3,2-f:3′,4′-i][1,4,13]oxadiazacyclohexadecine-4,17(5H,16H)-dione;
[17a(18)Z]-2,5,12-trimethyl-6,7,9,10,11,12-hexahydro-1H-13,15-(azenometheno)dipyrrolo[3,2-f:3′,4′-i][1,4,13]oxadiazacyclohexadecine-4,17(5H,16H)-dione;
[17a(18)Z]-2,5,12-trimethyl-6,7,9,10,11,12-hexahydro-1H-13,15-(azenometheno)dipyrrolo[3,2-f:3′,4′-i][1,4,11,13]oxatriazacyclohexadecine-4,17(5H,16H)-dione;
[16a(17)Z]-2,5,11-trimethyl-6,7,8,9,10,11-hexahydro-1H-12,14-(azenometheno)dipyrrolo[3,2-i:3′,4′-l][1,4,7]triazacyclopentadecine-4,16(5H,15H)-dione;
[16a(17)Z]-2,5,11-trimethyl-6,7,8,9,10,11-hexahydro-1H-12,14-(ethanediylidene)dipyrrolo[3,2-k:3′,4′-n][1,3,6,9]tetraazacyclopentadecine-4,16(5H,15H)-dione;
[16a(17)Z]-2,5,11-trimethyl-6,7,8,9,10,11-hexahydro-1H-12,14-(azenometheno)dipyrrolo[3,2-k:3′,4′-n][1,3,6,9]tetraazacyclopentadecine-4,16(5H,15H)-dione;
[16a(17)Z]-11-cyclopropyl-2,5-dimethyl-6,7,8,9,10,11-hexahydro-1H-12,14-(ethanediylidene)dipyrrolo[3,2-k:3′,4′-n][1,3,6,9]tetraazacyclopentadecine-4,16(5H,15H)-dione;
[16a(17)Z]-11-cyclopropyl-2-methyl-6,7,8,9,10,11-hexahydro-1H-12,14-(azenometheno)dipyrrolo[3,2-k:3′,4′-n][1,3,6,9]tetraazacyclopentadecine-4,16(5H,15H)-dione;
[10R,16a(17)Z]-2,5,10-trimethyl-6,7,8,9,10,11-hexahydro-1H-12,14-(azenometheno)dipyrrolo[3,2-i:3′,4′-l][1,4,7]triazacyclopentadecine-4,16(5H,15H)-dione;
[10S,16a(17)Z]-2,5,10-trimethyl-6,7,8,9,10,11-hexahydro-1H-12,14-(azenometheno)dipyrrolo[3,2-i:3′,4′-l][1,4,7]triazacyclopentadecine-4,16(5H,15H)-dione;
[10S,16a(17)Z]-2,5,10-trimethyl-6,7,10,11-tetrahydro-1H,9H-12,14-(azenometheno)dipyrrolo[3,2-f:3′,4′-i][1,4,13]oxadiazacyclopentadecine-4,16(5H,15H)-dione;
[10S,16a(17)Z]-2,5,10-trimethyl-6,7,10,11-tetrahydro-1H,9H-12,14-(azenometheno)dipyrrolo[3,4-g:2′,3′-j][1,4,6,13]oxatriazacyclopentadecine-4,16(5H,15H)-dione;
[10S,16a(17)Z]-2,5,10-trimethyl-6,7,9,10-tetrahydro-1H-12,14-(azenometheno)dipyrrolo[3,2-i:3′,4′-l][1,4,7]dioxazacyclopentadecine-4,16(5H,15H)-dione;
[10S,16a(17)Z]-2,5,10-trimethyl-6,7,9,10-tetrahydro-1H-12,14-(azenometheno)dipyrrolo[3,4-d:2′,3′-g][1,13,3,10]dioxadiazacyclopentadecine-4,16(5H,15H)-dione;
[10S,16a(17)Z]-2,5,10-trimethyl-6,7,9,10-tetrahydro-1H-12,14-(ethanediylidene)dipyrrolo[3,4-d:2′,3′-g][1,13,3,10]dioxadiazacyclopentadecine-4,16(5H,15H)-dione;
[10S,16a(17)Z]-2,5,10-trimethyl-5,6,7,8,9,10-hexahydro-12,14-(azenometheno)dipyrrolo[3,4-d:2′,3′-g][1,3,10,13]oxatriazacyclopentadecine-4,16(1H,15H)-dione;
[10S,16a(17)Z]-2,5,10-trimethyl-5,6,7,8,9,10-hexahydro-12,14-(ethanediylidene)dipyrrolo[3,4-d:2′,3′-g][1,3,10,13]oxatriazacyclopentadecine-4,16(1H,15H)-dione;
[10S,16a(17)Z]-2,5,10-trimethyl-5,6,7,8,9,10-hexahydro-12,14-(azenometheno)dipyrrolo[3,2-i:3′,4′-l][1,4,7]oxadiazacyclopentadecine-4,16(1H,15H)-dione;
[9R,16a(17)Z]-2,5,9-trimethyl-6,7,9,10-tetrahydro-1H-12,14-(azenometheno)dipyrrolo[3,4-d:2′,3′-g][1,13,3,10]dioxadiazacyclopentadecine-4,16(5H,15H)-dione;
[9S,16a(17)Z]-2,5,9-trimethyl-6,7,9,10-tetrahydro-1H-12,14-(azenometheno)dipyrrolo[3,4-d:2′,3′-g][1,13,3,10]dioxadiazacyclopentadecine-4,16(5H,15H)-dione;
[16a(17)Z]-2,5-dimethyl-6,7,9,10-tetrahydro-1H-12,14-(ethanediylidene)dipyrrolo[3,2-i:3′,4′-l][1,4,7]dioxazacyclopentadecine-4,16(5H,15H)-dione;
[10S,16a(17)Z]-2,5,10-trimethyl-6,7,9,10-tetrahydro-1H-12,14-(ethanediylidene)dipyrrolo[3,2-i:3′,4′-][1,4,7]dioxazacyclopentadecine-4,16(5H,15H)-dione;
[10R,16a(17)Z]-2,5,10-trimethyl-6,7,9,10-tetrahydro-1H-12,14-(ethanediylidene)dipyrrolo[3,2-i:3′,4′-l][1,4,7]dioxazacyclopentadecine-4,16(5H,15H)-dione;
[10S,16a(17)Z]-2,10-dimethyl-6,7,9,10-tetrahydro-1H-12,14-(ethanediylidene)dipyrrolo[3,2-i:3′,4′-l][1,4,7]dioxazacyclopentadecine-4,16(5H,15H)-dione;
[10S,16a(17)Z]-2,5,10-trimethyl-5,6,7,8,9,10-hexahydro-12,14-(ethanediylidene)dipyrrolo[3,2-i:3′,4′-l][1,4,7]oxadiazacyclopentadecine-4,16(1H,15H)-dione;
[9R,16a(17)Z]-2,5,9-trimethyl-6,7,9,10-tetrahydro-1H-12,14-(ethanediylidene)dipyrrolo[3,2-i:3′,4′-l][1,4,7]dioxazacyclopentadecine-4,16(5H,15H)-dione;
[9S,16a(17)Z]-2,5,9-trimethyl-6,7,9,10-tetrahydro-1H-12,14-(ethanediylidene)dipyrrolo[3,2-i:3′,4′-l][1,4,7]dioxazacyclopentadecine-4,16(5H,15H)-dione;
[17a(18)Z]-2-methyl-6,7,10,11-tetrahydro-1H,9H-13,15-(ethanediylidene)dipyrrolo[3,2-f:3′,4′-i][1,13,4]oxathiazacyclohexadecine-4,17(5H,16H)-dione;
[17a(18)Z]-2-methyl-6,7,10,11-tetrahydro-1H-13,15-(ethanediylidene)-12⁶-dipyrrolo[3,2-f:3′,4′-i][1,13,4]oxathiazacyclohexadecine-4,12,12,17(5H,9H,16H)-tetrone;
[17a(18)Z]-2-methyl-6,7,9,10-tetrahydro-1H,12H-13,15-(ethanediylidene)dipyrrolo[3,2-i:3′,4′-l][1,4,7]dioxazacyclohexadecine-4,17(5H,16H)-dione;
[12R,17a(18)Z]-2,12-dimethyl-6,7,9,10-tetrahydro-1H,12H-13,15-(ethanediylidene)dipyrrolo[3,2-i:3′,4′-l][1,4,7]dioxazacyclohexadecine-4,17(5H,16H)-dione;
[12S,17a(18)Z]-2,12-dimethyl-6,7,9,10-tetrahydro-1H,12H-13,15-(ethanediylidene)dipyrrolo[3,2-i:3′,4′-l][1,4,7]dioxazacyclohexadecine-4,17(5H,16H)-dione;
[12S,17a(18)Z]-2,5,12-trimethyl-6,7,9,10-tetrahydro-1H,12H-13,15-(ethanediylidene)dipyrrolo[3,2-i:3′,4′-l][1,4,7]dioxazacyclohexadecine-4,17(5H,16H)-dione;
[17a(18)Z]-2,5-dimethyl-6,7,11,12-tetrahydro-1H-13,15-(ethanediylidene)dipyrrolo[3,2-f:3′,4′-i][1,4,14]oxadiazacyclohexadecine-4,10,17(5H,9H,16H)-trione;
[17a(18)Z]-2,5-dimethyl-6,7,11,12-tetrahydro-1H-13,15-(azenometheno)dipyrrolo[3,4-h:2′,3′-k][1,4,7,14]oxatriazacyclohexadecine-4,10,17(5H,9H,16H)-trione;
[17a(18)Z]-2,5-dimethyl-6,7,11,12-tetrahydro-1H-13,15-(azenometheno)dipyrrolo[3,2-f:3′,4′-i][1,4,14]oxadiazacyclohexadecine-4,10,17(5H,9H,16H)-trione;
[17a(18)Z]-2,5-dimethyl-6,7,11,12-tetrahydro-1H-13,15-(ethanediylidene)dipyrrolo[3,4-h:2′,3′-k][1,4,7,14]oxatriazacyclohexadecine-4,10,17(5H,9H,16H)-trione;
[12S,17a(18)Z]-2,5,12-trimethyl-6,7,11,12-tetrahydro-1H-13,15-(ethanediylidene)dipyrrolo[3,4-h:2′,3′-k][1,4,7,14]oxatriazacyclohexadecine-4,10,17(5H,9H,16H)-trione;
[17a(18)Z]-2-methyl-6,7,11,12-tetrahydro-1H-13,15-(ethanediylidene)dipyrrolo[3,4-h:2′,3′-k][1,4,7,14]oxatriazacyclohexadecine-4,10,17(5H,9H,16H)-trione;
[17a(18)Z]-2,11-dimethyl-6,7,11,12-tetrahydro-1H-13,15-(ethanediylidene)dipyrrolo[3,4-h:2′,3′-k][1,4,7,14]oxatriazacyclohexadecine-4,10,17(5H,9H,16H)-trione;
[17a(18)Z]-2,11-dimethyl-6,7,11,12-tetrahydro-1H-13,15-(ethanediylidene)dipyrrolo[3,2-f:3′,4′-i][1,4,14]oxadiazacyclohexadecine-4,10,17(5H,9H,16H)-trione;
[17a(18)Z]-2,11-dimethyl-6,7,11,12-tetrahydro-1H-13,15-(azenometheno)dipyrrolo[3,4-h:2′,3′-k][1,4,7,14]oxatriazacyclohexadecine-4,10,17(5H,9H,16H)-trione;
[17a(18)Z]-2,11-dimethyl-6,7,11,12-tetrahydro-1H-13,15-(azenometheno)dipyrrolo[3,2-f:3′,4′-i][1,4,14]oxadiazacyclohexadecine-4,10,17(5H,9H,16H)-trione;
[18a(19)Z]-2-methyl-6,7,10,11-tetrahydro-1H,9H-14,16-(ethanediylidene)dipyrrolo[3,2-f:3′,4′-i][1,4,15]oxadiazacycloheptadecine-4,12,18(5H,13H,17H)-trione;
[18a(19)Z]-2,5-dimethyl-6,7,10,11-tetrahydro-1H,9H-14,16-(ethanediylidene)dipyrrolo[3,2-f:3′,4′-i][1,4,15]oxadiazacycloheptadecine-4,12,18(5H,13H,17H)-trione;
[18a(19)Z]-2,11-dimethyl-6,7,10,11-tetrahydro-1H,9H-14,16-(ethanediylidene)dipyrrolo[3,2-f:3′,4′-i][1,4,15]oxadiazacycloheptadecine-4,12,18(5H,13H,17H)-trione;
[13S,18a(19)Z]-2,13-dimethyl-6,7,10,11-tetrahydro-1H,9H-14,16-(ethanediylidene)dipyrrolo[3,2-f:3′,4′-i][1,4,15]oxadiazacycloheptadecine-4,12,18(5H,13H,17H)-trione;
[13R,18a(19)Z]-2,13-dimethyl-6,7,10,11-tetrahydro-1H,9H-14,16-(ethanediylidene)dipyrrolo[3,2-f:3′,4′-i][1,4,15]oxadiazacycloheptadecine-4,12,18(5H,13H,17H)-trione;
[18a(19)Z]-2-methyl-6,7,10,11-tetrahydro-1H,9H-14,16-(azenometheno)dipyrrolo[3,4-i:2′,3′-l][1,4,8,15]oxatriazacycloheptadecine-4,12,18(5H,13H,17H)-trione;
[13S,18a(19)Z]-2,13-dimethyl-6,7,10,11-tetrahydro-1H,9H-14,16-(ethanediylidene)dipyrrolo[3,4-i:2′,3′-l][1,4,8,15]oxatriazacycloheptadecine-4,12,18(5H,13H,17H)-trione;
[13S,18a(19)Z]-2,13-dimethyl-6,7,10,11-tetrahydro-1H,9H-14,16-(azenometheno)dipyrrolo[3,2-f:3′,4′-i][1,4,15]oxadiazacycloheptadecine-4,12,18(5H,13H,17H)-trione;
[13S,18a(19)Z]-13-hydroxy-2,13-dimethyl-6,7,10,11-tetrahydro-1H,9H-14,16-(ethanediylidene)dipyrrolo[3,2 f:3′,4′-i][1,4,15]oxadiazacycloheptadecine-4,12,18(5H,13H,17H)-trione;
[16a(17)Z]-2-methyl-6,7,9,10-tetrahydro-1H-12,14-(ethanediylidene)dipyrrolo[3,2-i:3′,4′-l][1,4,7]dioxazacyclopentadecine-4,16(5H,15H)-dione;
[16a(17)Z]-19-chloro-2-methyl-6,7,9,10-tetrahydro-1H-12,14-(ethanediylidene)dipyrrolo[3,2-i:3′,4′-l][1,4,7]dioxazacyclopentadecine-4,16(5H,15H)-dione;
[16a(17)Z]-19-chloro-2,5-dimethyl-6,7,9,10-tetrahydro-1H-12,14-(ethanediylidene)dipyrrolo[3,2-i:3′,4′-l][1,4,7]dioxazacyclopentadecine-4,16(5H,15H)-dione;
methyl[7R,16a(17)Z]-19-chloro-2,5-dimethyl-4,16-dioxo-4,5,6,7,9,10,15,16-octahydro-1H-12,14-(ethanediylidene)dipyrrolo[3,2-i:3′,4′-l][1,4,7]dioxazacyclopentadecine-7-carboxylate;
[7R,16a(17)Z]—N-(azetidin-3-yl)-19-chloro-2,5-dimethyl-4,16-dioxo-4,5,6,7,9,10,15,16-octahydro-1H-12,14-(ethanediylidene)dipyrrolo[3,2-i:3′,4′-l][1,4,7]dioxazacyclopentadecine-7-carboxamide;
[7R,16a(17)Z]-19-chloro-2,5-dimethyl-4,16-dioxo-N-(piperidin-4-yl)-4,5,6,7,9,10,15,16-octahydro-1H-12,14-(ethanediylidene)dipyrrolo[3,2-i:3′,4′-l][1,4,7]dioxazacyclopentadecine-7-carboxamide;
[7R,16a(17)Z]-19-chloro-N,2,5-trimethyl-4,16-dioxo-4,5,6,7,9,10,15,16-octahydro-1H-12,14-(ethanediylidene)dipyrrolo[3,2-i:3′,4′-l][1,4,7]dioxazacyclopentadecine-7-carboxamide;
[7R,16a(17)Z]-19-chloro-2,5-dimethyl-4,16-dioxo-N-[(3R)-pyrrolidin-3-yl]-4,5,6,7,9,10,15,16-octahydro-1H-12,14-(ethanediylidene)dipyrrolo[3,2-i:3′,4′-l][1,4,7]dioxazacyclopentadecine-7-carboxamide;
[7R,16a(17)Z]-19-chloro-N,N,2,5-tetramethyl-4,16-dioxo-4,5,6,7,9,10,15,16-octahydro-1H-12,14-(ethanediylidene)dipyrrolo[3,2-i:3′,4′-l][1,4,7]dioxazacyclopentadecine-7-carboxamide;
[7R,16a(17)Z]-19-chloro-2,5-dimethyl-7-(4-methylpiperazine-1-carbonyl)-6,7,9,10-tetrahydro-1H-12,14-(ethanediylidene)dipyrrolo[3,2-i:3′,4′-l][1,4,7]dioxazacyclopentadecine-4,16(5H,15H)-dione;
[10S,16a(17)Z]-2,5,10-trimethyl-6,7,9,10-tetrahydro-1H-12,14-(ethanediylidene)dipyrrolo[3,2-i:3′,4′-l][1,4,7]dioxazacyclopentadecine-4,16(5H,15H)-dione;
[10S,16a(17)Z]-19-chloro-2,5,10-trimethyl-6,7,9,10-tetrahydro-1H-12,14-(ethanediylidene)dipyrrolo[3,2-i:3′,4′-l][1,4,7]dioxazacyclopentadecine-4,16(5H,15H)-dione;
[16a(17)Z]-19-chloro-5-methyl-6,7,9,10-tetrahydro-1H-12,14-(ethanediylidene)dipyrrolo[3,2-i:3′,4′-l][1,4,7]dioxazacyclopentadecine-4,16(5H,15H)-dione;
[16a(17)Z]-19-chloro-2,5-dimethyl-6,7,9,10-tetrahydro-1H-12,14-(ethanediylidene)dipyrrolo[3,2-i:3′,4′-l][1,4,7]oxathiazacyclopentadecine-4,16(5H,15H)-dione;
[16a(17)Z]-19-chloro-2,5-dimethyl-5,6,7,8,9,10-hexahydro-12,14-(ethanediylidene)dipyrrolo[3,2-i:3′,4′-l][1,4,7]oxadiazacyclopentadecine-4,16(1H,15H)-dione;
[16a(17)Z]-19-chloro-2,5-dimethyl-6,7,9,10-tetrahydro-12,14-(ethanediylidene)-8λ⁶-dipyrrolo[3,2-i:3′,4′-l][1,4,7]oxathiazacyclopentadecine-4,8,8,16(1H,5H,15H)-tetrone;
[16a(17)Z]-19-chloro-2,5-dimethyl-6,7,9,10-tetrahydro-12,14-(ethanediylidene)-8k-dipyrrolo[3,2-i:3′,4′-l][1,4,7]oxathiazacyclopentadecine-4,8,16(1H,5H,15H)-trione;
[16a(17)Z]-19-chloro-5-methyl-6,7,9,10-tetrahydro-1H-12,14-(ethanediylidene)dipyrrolo[3,2-i:3′,4′-l][1,4,7]oxathiazacyclopentadecine-4,16(5H,15H)-dione;
[16a(17)Z]-2,5-dimethyl-6,7,9,10-tetrahydro-1H-12,14-(ethanediylidene)dipyrrolo[3,4-g:2′,3′-j][1,4,13]oxathiazacyclopentadecine-4,16(5H,15H)-dione;
[16a(17)Z]-2,5-dimethyl-6,7-dihydro-1H,9H-12,14-(ethanediylidene)-11λ⁶-dipyrrolo[3,4-g:2′,3′-j][1,4,13]oxathiazacyclopentadecine-4,11,11,16(5H,10H,15H)-tetrone;
[16a(17)Z]-5-methyl-6,7,9,10-tetrahydro-1H-12,14-(ethanediylidene)dipyrrolo[3,4-g:2′,3′-j][1,4,13]oxathiazacyclopentadecine-4,16(5H,15H)-dione;
[16a(17)Z]-19-chloro-5-methyl-6,7,9,10-tetrahydro-1H-12,14-(ethanediylidene)pyrazolo[4,3-i]pyrrolo[3,4-l][1,4,7]dioxazacyclopentadecine-4,16(5H,15H)-dione;
[16a(17)Z]-19-chloro-5-methyl-5,6,7,8,9,10-hexahydro-12,14-(ethanediylidene)dipyrrolo[3,2-i:3′,4′-][1,4,7]oxadiazacyclopentadecine-4,16(1H,15H)-dione;
[16a(17)Z]-19-chloro-2,5,8-trimethyl-5,6,7,8,9,10-hexahydro-12,14-(ethanediylidene)dipyrrolo[3,2-i:3′,4′-][1,4,7]oxadiazacyclopentadecine-4,16(1H,15H)-dione;
[16a(17)Z]-2,5-dimethyl-4,16-dioxo-4,5,6,7,9,10,15,16-octahydro-1H-12,14-(ethanediylidene)dipyrrolo[3,2-i:3′,4′-l][1,4,7]dioxazacyclopentadecine-19-carbonitrile;
[16a(17)Z]-19-chloro-2,5-dimethyl-6,7,9,10-tetrahydro-1H-12,14-(ethanediylidene)dipyrrolo[3,4-g:2′,3′-j][1,4,13]oxathiazacyclopentadecine-4,16(5H,15H)-dione;
[16a(17)Z]-19-chloro-5-methyl-6,7,9,10-tetrahydro-1H-12,14-(ethanediylidene)dipyrrolo[3,4-g:2′,3′-j][1,4,13]oxathiazacyclopentadecine-4,16(5H,15H)-dione; and
[16a(17)Z]-19-chloro-5-methyl-6,7-dihydro-1H,9H-12,14-(ethanediylidene)-11λ⁶-dipyrrolo[3,4-g:2′,3′-j][1,4,13]oxathiazacyclopentadecine-4,11,11,16(5H,10H,15H)-tetrone.
   or a pharmaceutically acceptable salt thereof.

The following represent illustrative embodiments of compounds of Formula (I):

| Example | Structure | Name |
|---|---|---|
| 1 | 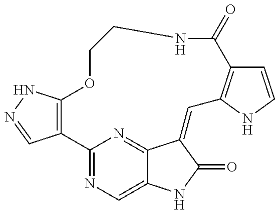 [SMILES: O=C1Nc2cnc3nc2/C1=C/c1[nH]ccc1C(=O)NCCOc1[nH]ncc1-3] | [3a(4)Z]-10,11-dihydro-2H,13H-16,1- (azenometheno)pyrazolo[4,3- m]dipyrrolo[3,2-f:3′,4′- i][1,4,11]oxadiazacyclotetradecine- 3,8(5H,9H)-dione |
| 2 | 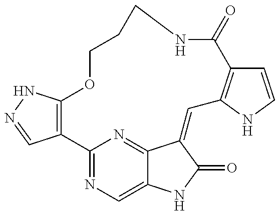 [SMILES: O=C1Nc2cnc3nc2/C1=C/c1[nH]ccc1C(=O)NCCCOc1[nH]ncc1-3] | [3a(4)Z]-9,10,11,12-tetrahydro-14H- 17,1-(azenometheno)pyrazolo[3,4- b]dipyrrolo[3,4-f:2′,3′- i][1,5,12]oxadiazacyclopentadecine- 3,8(2H,5H)-dione |
| 3 | 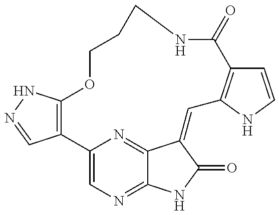 [SMILES: O=C1Nc2ncc3nc2/C1=C/c1[nH]ccc1C(=O)NCCCOc1[nH]ncc1-3] | [3a(4)Z]-9,10,11,12-tetrahydro-14H- 1,17-(azenometheno)pyrazolo[3,4- b]dipyrrolo[3,4-f:2′,3′- i][1,5,12]oxadiazacyclopentadecine- 3,8(2H,5H)-dione |
| 4 | 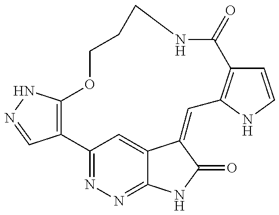 [SMILES: O=C1Nc2nnc3cc2/C1=C/c1[nH]ccc1C(=O)NCCCOc1[nH]ncc1-3] | [3a(4)Z]-9,10,11,12-tetrahydro-14H- 1,17-(diazanediylidene)pyrazolo[4,3- n]dipyrrolo[3,2-g:3′,4′- j][1,5]oxazaacyclopentadecine- 3,8(2H,5H)-dione |
| 5 | 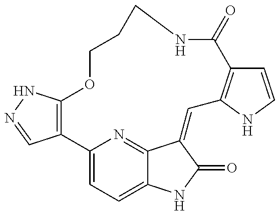 [SMILES: O=C1Nc2ccc3nc2/C1=C/c1[nH]ccc1C(=O)NCCCOc1[nH]ncc1-3] | [3a(4)Z]-9,10,11,12-tetrahydro-14H- 1,17-(ethanediylidene)pyrazolo[3,4- b]dipyrrolo[3,4-f:2′,3′- i][1,5,12]oxadiazacyclopentadecine- 3,8(2H,5H)-dione |
| 6 | 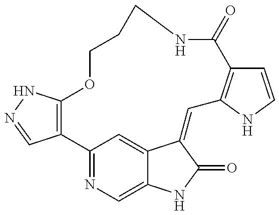 [SMILES: O=C1Nc2cnc3cc2/C1=C/c1[nH]ccc1C(=O)NCCCOc1[nH]ncc1-3] | [3a(4)Z]-9,10,11,12-tetrahydro-14H- 17,1-(azenometheno)pyrazolo[4,3- n]dipyrrolo[3,2-g:3′,4′- j][1,5]oxazacyclopentadecine- 3,8(2H,5H)-dione |
| 7 | 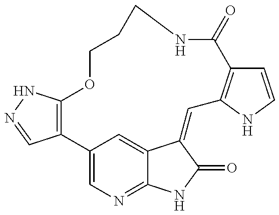 [SMILES: O=C1Nc2ncc3cc2/C1=C/c1[nH]ccc1C(=O)NCCCOc1[nH]ncc1-3] | [3a(4)Z]-9,10,11,12-tetrahydro-14H- 1,17-(azenometheno)pyrazolo[4,3- n]dipyrrolo[3,2-g:3′,4′- j][1,5]oxazacyclopentadecine- 3,8(2H,5H)-dione |
| 8 | 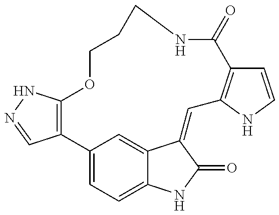 [SMILES: O=C1Nc2ccc3cc2/C1=C/c1[nH]ccc1C(=O)NCCCOc1[nH]ncc1-3] | [3a(4)Z]-9,10,11,12-tetrahydro-14H- 1,17-(ethanediylidene)pyrazolo[4,3- n]dipyrrolo[3,2-g:3′,4′- j][1,5]oxazacyclopentadecine- 3,8(2H,5H)-dione |
| 9 | 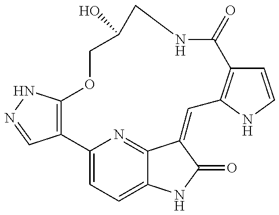 [SMILES: O=C1Nc2ccc3nc2/C1=C/c1[nH]ccc1C(=O)NC[C@H](O)COc1[nH]ncc1-3] | [3a(r)Z,11S]-11-hydroxy-9,10,11,12- tetrahydro-14H-1,17- (ethanediylidene)pyrazolo[3,4- b]dipyrrolo[3,4-f:2′,3′- i][1,5,12]oxadiazacyclopentadecine- 3,8(2H,5H)-dione |
| 10 | 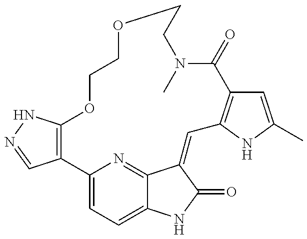 [SMILES: Cc1cc2c([nH]1)/C=C1\C(=O)Nc3ccc(nc31)-c1cn[nH]c1OCCOCCN(C)C2=O] | [19a(2))Z]-2,5-dimethyl-6,7,9,10- tetrahydro-1H,12H-15,17- (ethanediylidene)pyrazolo[4,3- p]dipyrrolo[3,2-i:3′,4′- l][1,4,7,14]dioxadiazacycloheptadecine- 4,19(5H,18H)-dione |
| 11 | 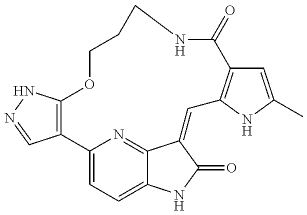 [SMILES: Cc1cc2c([nH]1)/C=C1\C(=O)Nc3ccc(nc31)-c1cn[nH]c1OCCCNC2=O] | [3a(4)Z]-6-methyl-9,10,11,12- tetrahydro-14H-1,17- (ethanediylidene)pyrazolo[3,4- b]dipyrrolo[3,4-f:2′,3′- i][1,5,12]oxadiazacyclopentadecine- 3,8(2H,5H)-dione |
| 12 | 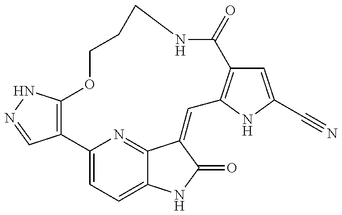 [SMILES: N#Cc1cc2c([nH]1)/C=C1\C(=O)Nc3ccc(nc31)-c1cn[nH]c1OCCCNC2=O] | [3a(4)Z]-3,8-dioxo-2,3,5,8,9,10,11,12- octahydro-14H-1,17- (ethanediylidene)pyrazolo[3,4- b]dipyrrolo[3,4-f:2′,3′- i][1,5,12]oxadiazacyclopentadecine-6- carbonitrile |
| 13 | 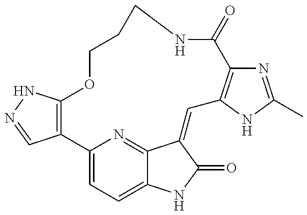 [SMILES: Cc1nc2c([nH]1)/C=C1\C(=O)Nc3ccc(nc31)-c1cn[nH]c1OCCCNC2=O] | [3a(4)Z]-6-methyl-9,10,11,12- tetrahydro-14H-1,17- (ethanediylidene)imidazo[4,5- i]pyrazolo[3,4-b]pyrrolo[3,4- f][1,5,12]oxadiazacyclopentadecine- 3,8(2H,5H)-dione |
| 14 | 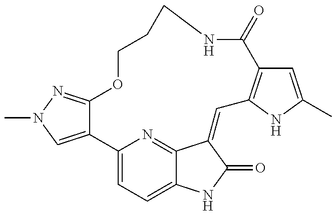 | [3a(4)Z]-6,15-dimethyl-9,10,11,12- tetrahydro-15H-1,17- (ethanediylidene)pyrazolo[3,4- b]dipyrrolo[3,4-f:2′,3′- i][1,5,12]oxadiazacyclopentadecine- 3,8(2H,5H)-dione |
| 15 | 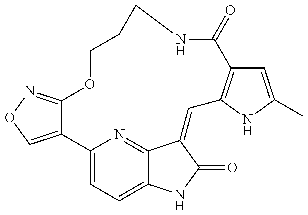 [SMILES: Cc1cc2c([nH]1)/C=C1\C(=O)Nc3ccc(nc31)-c1conc1OCCCNC2=O] | [3a(4)Z]-6-methyl-9,10,11,12- tetrahydro-1,17- (ethanediylidene)[1,2]oxazolo[3,4- b]dipyrrolo[3,4-f:2′,3′- i][1,5,12]oxadiazacyclopentadecine- 3,8(2H,5H)-dione |
| 16 | 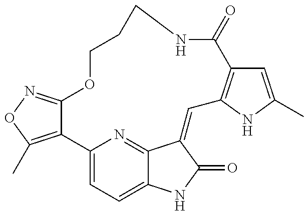 [SMILES: Cc1cc2c([nH]1)/C=C1\C(=O)Nc3ccc(nc31)-c1c(noc1C)OCCCNC2=O] | [3a(4)Z]-6,16-dimethyl-9,10,11,12- tetrahydro-1,17- (ethanediylidene)[1,2]oxazolo[3,4- b]dipyrrolo[3,4-f:2′,3′- i][1,5,12]oxadiazacyclopentadecine- 3,8(2H,5H)-dione |
| 17 | 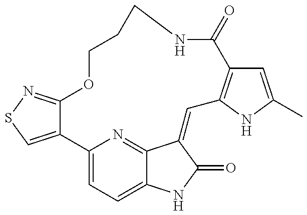 [SMILES: Cc1cc2c([nH]1)/C=C1\C(=O)Nc3ccc(nc31)-c1csnc1OCCCNC2=O] | [3a(4)Z]-6-methyl-9,10,11,12- tetrahydro-1,17- (ethanediylidene)dipyrrolo[3,4-f:2′,3′- i][1,2]thiazolo[3,4- b][1,5,12]oxadiazacyclopentadecine- 3,8(2H,5H)-dione |
| 18 | 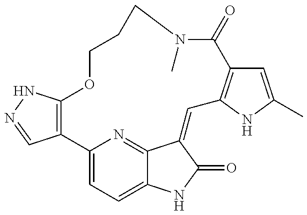 [SMILES: Cc1cc2c([nH]1)/C=C1\C(=O)Nc3ccc(nc31)-c1cn[nH]c1OCCCN(C)C2=O] | [3a(4)Z]-6,9-dimethyl-9,10,11,12- tetrahydro-14H-1,17- (ethanediylidene)pyrazolo[3,4- b]dipyrrolo[3,4-f:2′,3′- i][1,5,12]oxadiazacyclopentadecine- 3,8(2H,5H)-dione |
| 19 | 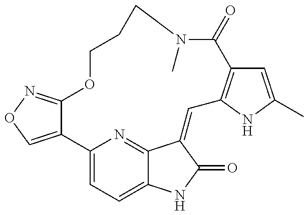 [SMILES: Cc1cc2c([nH]1)/C=C1\C(=O)Nc3ccc(nc31)-c1conc1OCCCN(C)C2=O] | [3a(4)Z]-6,9-dimethyl-9,10,11,12- tetrahydro-1,17- (ethanediylidene)[1,2]oxazolo[3,4- b]dipyrrolo[3,4-f:2′,3′- i][1,5,12]oxadiazacyclopentadecine- 3,8(2H,5H)-dione |
| 20 | 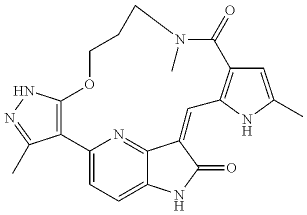 [SMILES: Cc1cc2c([nH]1)/C=C1\C(=O)Nc3ccc(nc31)-c1c(C)n[nH]c1OCCCN(C)C2=O] | [3a(4)Z]-6,9,16-trimethyl-9,10,11,12- tetrahydro-14H-1,17- (ethanediylidene)pyrazolo[3,4- b]dipyrrolo[3,4-f:2′,3′- i][1,5,12]oxadiazacyclopentadecine- 3,8(2H,5H)-dione |
| 21 | 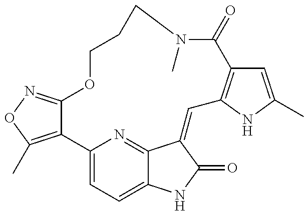 [SMILES: Cc1cc2c([nH]1)/C=C1\C(=O)Nc3ccc(nc31)-c1c(noc1C)OCCCN(C)C2=O] | [3a(4)Z]-6,9,16-trimethyl-9,10,11,12- tetrahydro-1,17- (ethanediylidene)[1,2]oxazolo[3,4- b]dipyrrolo[3,4-f:2′,3′- i][1,5,12]oxadiazacyclopentadecine- 3,8(2H,5H)-dione |
| 22 | 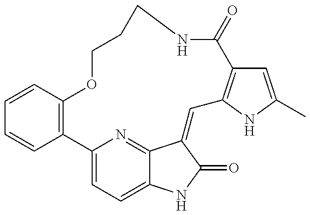 [SMILES: Cc1cc2c([nH]1)/C=C1\C(=O)Nc3ccc(nc31)-c1ccccc1OCCCNC2=O] | [3a(4)Z]-6-methyl-9,10,11,12- tetrahydro-1,18- (ethanediylidene)dipyrrolo[3,2-g:3′,4′- j][1,5,12]benzoxadiazacyclopentadecine- 3,8(2H,5H)-dione |
| 23 | 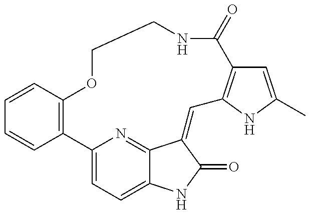 [SMILES: Cc1cc2c([nH]1)/C=C1\C(=O)Nc3ccc(nc31)-c1ccccc1OCCNC2=O] | [3a(4)Z]-6-methyl-10,11-dihydro-2H- 1,17-(ethanediylidene)dipyrrolo[3,2- f:3′,4′- i][1,4,11]benzoxadiazacyclotetradecine- 3,8(5H,9H)-dione |
| 24 | 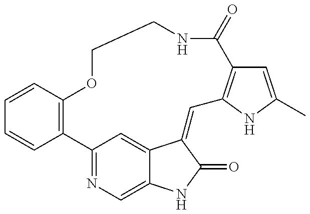 [SMILES: Cc1cc2c([nH]1)/C=C1\C(=O)Nc3cnc(cc31)-c1ccccc1OCCNC2=O] | [3a(4)Z]-6-methyl-10,11-dihydro-2H- 17,1-(azenomethano)dipyrrolo[3,2- f:3′,4′-i][1,4]benzoxazacyclotetradecine- 3,8(5H,9H)-dione |
| 25 | 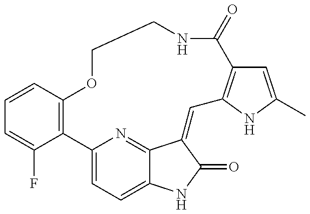 [SMILES: Cc1cc2c([nH]1)/C=C1\C(=O)Nc3ccc(nc31)-c1c(F)cccc1OCCNC2=O] | [3a(4)Z]-16-fluoro-6-methyl-10,11- dihydro-2H-1,17- (ethanediylidene)dipyrrolo[3,2-f:3′,4′- i][1,4,11]benzoxadiazacyclotetradecine- 3,8(5H,9H)-dione |
| 26 | 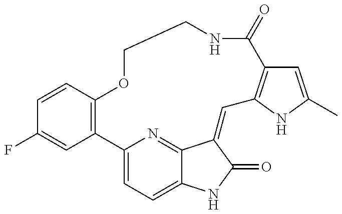 [SMILES: Cc1cc2c([nH]1)/C=C1\C(=O)Nc3ccc(nc31)-c1cc(F)ccc1OCCNC2=O] | [3a(4)Z]-15-fluoro-6-methyl-10,11- dihydro-2H-1,17- (ethanediylidene)dipyrrolo[3,2-f:3′,4′- i][1,4,11]benzoxadiazacyclotetradecine- 3,8(5H,9H)-ione |
| 27 | 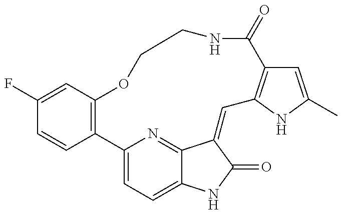 [SMILES: Cc1cc2c([nH]1)/C=C1\C(=O)Nc3ccc(nc31)-c1ccc(F)cc1OCCNC2=O] | [3a(4)Z]-14-fluoro-6-methyl-10,11- dihydro-2H-1,17- (ethanediylidene)dipyrrolo[3,2-f:3′,4′- i][1,4,11]benzoxadiazacyclotetradecine- 3,8(5H,9H)-dione |
| 28 | 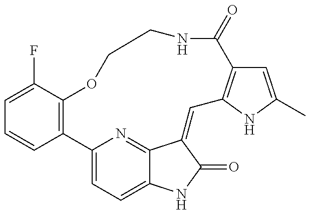 [SMILES: Cc1cc2c([nH]1)/C=C1\C(=O)Nc3ccc(nc31)-c1cccc(F)c1OCCNC2=O] | [3a(4)Z]-13-fluoro-6-methyl-10,11- dihydro-2H-1,17- (ethanediylidene)dipyrrolo[3,2-f:3′,4′- i][1,4,11]benzoxadiazacyclotetradecein- 3,8(5H,9H)-dione |
| 29 | 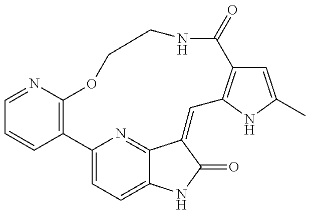 [SMILES: Cc1cc2c([nH]1)/C=C1\C(=O)Nc3ccc(nc31)-c1cccnc1OCCNC2=O] | [3a(4)Z]-6-methyl-10,11-dihydro-2H- 1,17-(ethanediylidene)pyrido[3,2- m]dipyrrolo[3,2-f:3′,4′- i][1,4,11]oxadiazacyclotetradecine- 3,8(5H,9H)-dione |
| 30 | 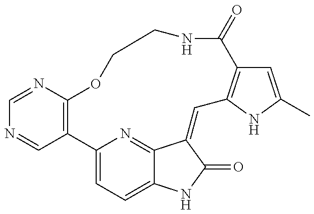 [SMILES: Cc1cc2c([nH]1)/C=C1\C(=O)Nc3ccc(nc31)-c1cncnc1OCCNC2=O] | [3a(4)Z]-6-methyl-10,11-dihydro-2H- 1,17-(ethanediylidene)pyrimido[5,4- m]dipyrrolo[3,2-f:3′,4′- i][1,4,11]oxadiazacyclotetradecine- 3,8(5H,9H)-dione |
| 31 | 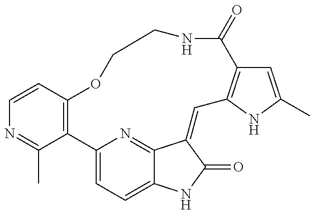 [SMILES: Cc1cc2c([nH]1)/C=C1\C(=O)Nc3ccc(nc31)-c1c(ccnc1C)OCCNC2=O] | [3a(4)Z]-6,16-dimethyl-10,11-dihydro- 2H-1,17-(ethanediylidene)pyrido[3,4- m]dipyrrolo[3,2-f:3′,4′- i][1,4,11]oxadiazacyclotetradecine- 3,8(5H,9H)-dione |
| 32 | 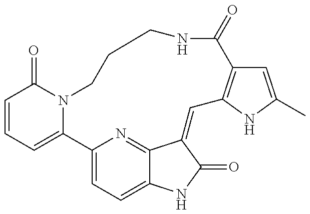 [SMILES: Cc1cc2c([nH]1)/C=C1\C(=O)Nc3ccc(nc31)-c1cccc(=O)n1CCCNC2=O] | [3a(4)Z]-6-methyl-9,10,11,12- tetrahydro-14H-1,18- (ethanediylidene)pyrido[2,1- c]dipyrrolo[3,2-j:3′,4′- m][1,4,8]triazacyclotetradecine- 3,8,14(2H,5H)-trione |
| 33 | 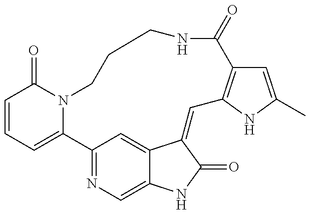 [SMILES: Cc1cc2c([nH]1)/C=C1\C(=O)Nc3cnc(cc31)-c1cccc(=O)n1CCCNC2=O] | [3a(4)Z]-6-methyl-9,10,11,12- tetrahydro-14H-18,1- (azenometheno)pyrido[1,2- e]dipyrrolo[3,4-i:2′,3′- l][1,5]diazacyclotetradecine- 3,8,14(2H,5H)-trione |
| 34 | 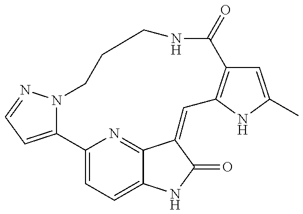 [SMILES: Cc1cc2c([nH]1)/C=C1\C(=O)Nc3ccc(nc31)-c1ccnn1CCCNC2=O] | [3a(4)Z]-6-methyl-9,10,11,12- tetrahydro-1,17- (ethanediylidene)pyrazolo[5,1- c]dipyrrolo[3,2-j:3′,4′- m][1,4,8]triazacyclotetradecine- 3,8(2H,5H)-dione |
| 35 | 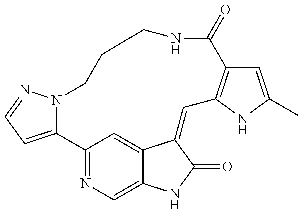 [SMILES: Cc1cc2c([nH]1)/C=C1\C(=O)Nc3cnc(cc31)-c1ccnn1CCCNC2=O] | [3a(4)Z]-6-methyl-9,10,11,12- tetrahydro-17,1- (azenometheno)pyrazolo[1,5- e]dipyrrolo[3,4-i:2′,3′- l][1,5]diazacyclotetradecine- 3,8(2H,5H)-dione |
| 36 | 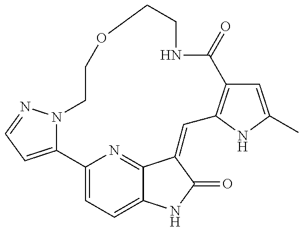 [SMILES: Cc1cc2c([nH]1)/C=C1\C(=O)Nc3ccc(nc31)-c1ccnn1CCOCCNC2=O] | [19a(2)Z]-2-methyl-6,7,9,10- tetrahydro-1H-15,17- (ethanediylidene)pyrazolo[1,5- d]dipyrrolo[3,4-h:2′,3′- k][1,4,7,14]oxatriazacyclohexadecine- 4,19(5H,18H)-dione |
| 37 | 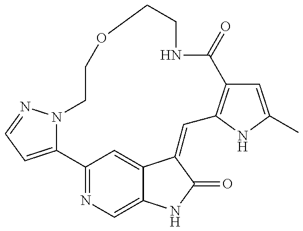 [SMILES: Cc1cc2c([nH]1)/C=C1\C(=O)Nc3cnc(cc31)-c1ccnn1CCOCCNC2=O] | [19a(2))Z]-2-methyl-6,7,9,10- tetrahydro-1H-15,17- (azenometheno)pyrazolo[1,5- d]dipyrrolo[3,4-h:2′,3′- k][1,4,14]oxadiazacyclohexadecine- 4,19(5H,18H)-dione |
| 38 | 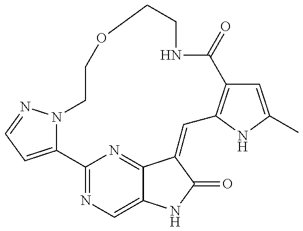 [SMILES: Cc1cc2c([nH]1)/C=C1\C(=O)Nc3cnc(nc31)-c1ccnn1CCOCCNC2=O] | [19a(20)Z]-2-methyl-6,7,9,10- tetrahydro-1H-15,17- (azenometheno)pyrazolo[1,5- d]dipyrrolo[3,4-h:2′,3′- k][1,4,7,14]oxatriazacyclohexadecine- 4,19(5H,18H)-dione |
| 39 | 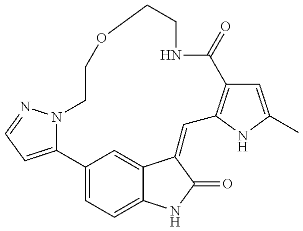 [SMILES: Cc1cc2c([nH]1)/C=C1\C(=O)Nc3ccc(cc31)-c1ccnn1CCOCCNC2=O] | [19a(20)Z]-2-methyl-6,7,9,10- tetrahydro-1H-15,17- (ethanediylidene)pyrazolo[1,5- d]dipyrrolo[3,4-h:2′,3′- k][1,4,14]oxadiazacyclohexadecine- 4,19(5H,18H)-dione |
| 40 | 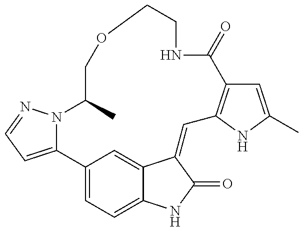 [SMILES: Cc1cc2c([nH]1)/C=C1\C(=O)Nc3ccc(cc31)-c1ccnn1[C@H](C)COCCNC2=O] | [10R,19a(20)Z]-2,10-dimethyl-6,7,9,10- tetrahydro-1H-15,17- (ethanediylidene)pyrazolo[1,5- d]dipyrrolo[3,4-h:2′,3′- k][1,4,14]oxadiazacyclohexadecine- 4,19(5H,18H)-dione |
| 41 | 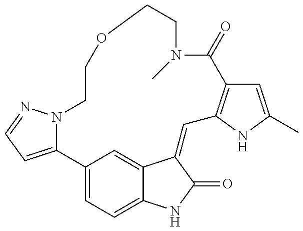 [SMILES: Cc1cc2c([nH]1)/C=C1\C(=O)Nc3ccc(cc31)-c1ccnn1CCOCCN(C)C2=O] | [19a(20)Z]-2,5-dimethyl-6,7,9,10- tetrahydro-1H-15,17- (ethanediylidene)pyrazolo[1,5- d]dipyrrolo[3,4-h:2′,3′- k][1,4,14]oxadiazacyclohexadecine- 4,19(5H,18H)-dione |
| 42 | 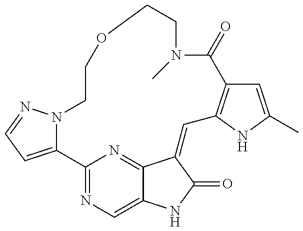 [SMILES: Cc1cc2c([nH]1)/C=C1\C(=O)Nc3cnc(nc31)-c1ccnn1CCOCCN(C)C2=O] | [19a(20)Z]-2,5-dimethyl-6,7,9,10- tetrahydro-1H-15,17- (azenometheno)pyrazolo[1,5- d]dipyrrolo[3,4-h:2′,3′- k][1,4,7,14]oxatriazacyclohexadecine- 4,19(5H,18H)-dione |
| 43 | 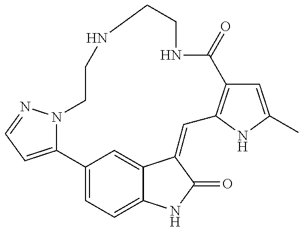 [SMILES: Cc1cc2c([nH]1)/C=C1\C(=O)Nc3ccc(cc31)-c1ccnn1CCNCCNC2=O] | [19a(20)Z]-2-methyl-5,6,7,8,9,10- hexahydro-15,17- (ethanediylidene)pyrazolo[1,5- g]dipyrrolo[3,4-k:2′,3′- n][1,4,7]triazacyclohexadecine- 4,19(1H,18H)-dione |
| 44 | 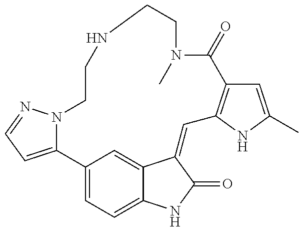 [SMILES: Cc1cc2c([nH]1)/C=C1\C(=O)Nc3ccc(cc31)-c1ccnn1CCNCCN(C)C2=O] | [19a(20)Z]-2,5-dimethyl-5,6,7,8,9,10- hexahydro-15,17- (ethanediylidene)pyrazolo[1,5- g]dipyrrolo[3,4-k:2′,3′- n][1,4,7]triazacyclohexadecine- 4,19(1H,18H)-dione |
| 45 | 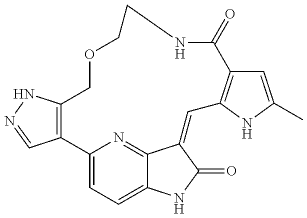 [SMILES: Cc1cc2c([nH]1)/C=C1\C(=O)Nc3ccc(nc31)-c1cn[nH]c1COCCNC2=O] | [3a(4)Z]-6-methyl-10,11,13,14- tetrahydro-2H-1,17- (ethanediylidene)pyrazolo[4,3- m]dipyrrolo[3,4-f:3′,4′- i][1,4,11]oxadiazacyclopentadecine- 3,8(5H,9H)-dione |
| 46 | 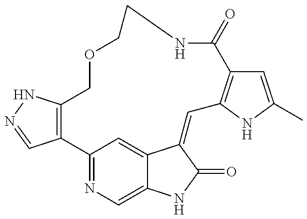 [SMILES: Cc1cc2c([nH]1)/C=C1\C(=O)Nc3cnc(cc31)-c1cn[nH]c1COCCNC2=O] | [3a(4)Z]-6-methyl-10,11,13,14- tetrahydro-2H-17,1- (azenometheno)pyrazolo[4,3- m]dipyrrolo[3,2-f:3′,4′- i][1,4]oxazacyclopentadecine- 3,8(5H,9H)-dione |
| 47 | 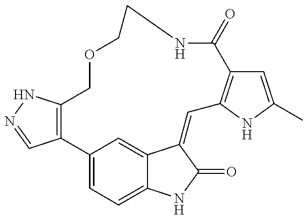 [SMILES: Cc1cc2c([nH]1)/C=C1\C(=O)Nc3ccc(cc31)-c1cn[nH]c1COCCNC2=O] | [3a(4)Z]-6-methyl-10,11,13,14- tetrahydro-2H-1,17- (ethanediylidene)pyrazolo[4,3- m]dipyrrolo[3,2-f:3′,4′- i][1,4]oxazacyclopentadecine- 3,8(5H,9H)-dione |
| 48 | 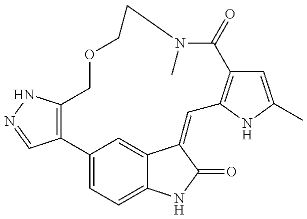 [SMILES: Cc1cc2c([nH]1)/C=C1\C(=O)Nc3ccc(cc31)-c1cn[nH]c1COCCN(C)C2=O] | [3a(4)Z]-6,9-dimethyl-10,11,13,14- tetrahydro-2H-1,17- (ethanediylidene)pyrazolo[4,3- m]dipyrrolo[3,2-f:3′,4′- i][1,4]oxazacyclopentadecine- 3,8(5H,9H)-dione |
| 49 | 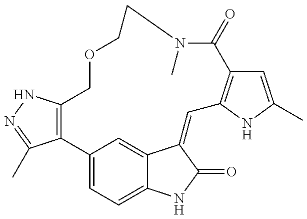 [SMILES: Cc1cc2c([nH]1)/C=C1\C(=O)Nc3ccc(cc31)-c1c(C)n[nH]c1COCCN(C)C2=O] | [3a(4)Z]-6,9,16-trimethyl-10,11,13,14- tetrahydro-2H-1,17- (ethanediylidene)pyrazolo[4,3- m]dipyrrolo[3,2-f:3′,4′- i][1,4]oxazacyclopentadecine- 3,8(5H,9H)-dione |
| 50 | 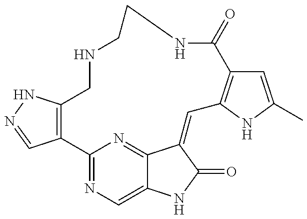 [SMILES: Cc1cc2c([nH]1)/C=C1\C(=O)Nc3cnc(nc31)-c1cn[nH]c1CNCCNC2=O] | [3a(4)Z]-6-methyl-9,10,11,12,13,14- hexahydro-17,1- (azenometheno)pyrazolo[3,4- f]dipyrrolo[3,4-j:2′,3′- m][1,4,9]triazacyclopentadecine- 3,8(2H,5H)-dione |
| 51 | 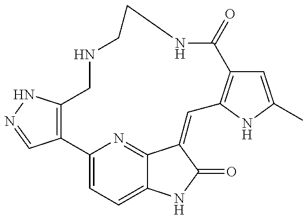 [SMILES: Cc1cc2c([nH]1)/C=C1\C(=O)Nc3ccc(nc31)-c1cn[nH]c1CNCCNC2=O] | [3a(4)Z]-6-methyl-9,10,11,12,13,14- hexahydro-1,17- (ethanediylidene)pyrazolo[3,4- f]dipyrrolo[3,4-j:2′,3′- m][1,4,9]triazacyclopentadecine- 3,8(2H,5H)-dione |
| 52 | 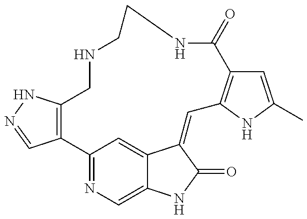 [SMILES: Cc1cc2c([nH]1)/C=C1\C(=O)Nc3cnc(cc31)-c1cn[nH]c1CNCCNC2=O] | [3a(4)Z]-6-methyl-9,10,11,12,13,14- hexahydro-17,1- (azenometheno)pyrazolo[4,3- m]dipyrrolo[3,2-f:3′,4′- i][1,4]diazacyclopentadecine- 3,8(2H,5H)-dione |
| 53 | 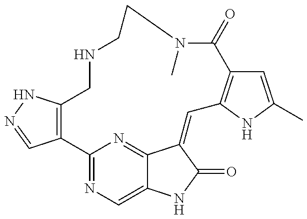 [SMILES: Cc1cc2c([nH]1)/C=C1\C(=O)Nc3cnc(nc31)-c1cn[nH]c1CNCCN(C)C2=O] | [3a(4)Z]-6,9-dimethyl-9,10,11,12,13,14- hexahydro-17,1- (azenometheno)pyrazolo[3,4- f]dipyrrolo[3,4-j:2′,3′- m][1,4,9]triazacyclopentadecine- 3,8(2H,5H)-dione |
| 54 | 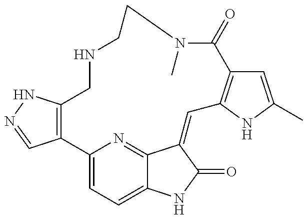 [SMILES: Cc1cc2c([nH]1)/C=C1\C(=O)Nc3ccc(nc31)-c1cn[nH]c1CNCCN(C)C2=O] | [3a(4)Z]-6,9-dimethyl-9,10,11,12,13,14- hexahydro-1,17- (ethanediylidene)pyrazolo[3,4- f]dipyrrolo[3,4-j:2′,3′- m][1,4,9]triazacyclopentadecine- 3,8(2H,5H)-dione |
| 55 | 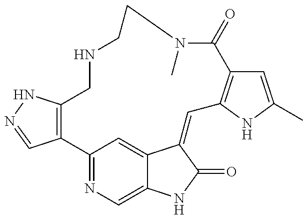 [SMILES: Cc1cc2c([nH]1)/C=C1\C(=O)Nc3cnc(cc31)-c1cn[nH]c1CNCCN(C)C2=O] | [3a(4)Z]-6,9-dimethyl-9,10,11,12,13,14- hexahydro-17,1- (azenometheno)pyrazolo[4,3- m]dipyrrolo[3,2-f:3′,4′- i][1,4]diazacyclopentadecine- 3,8(2,5H)-dione |
| 56 | 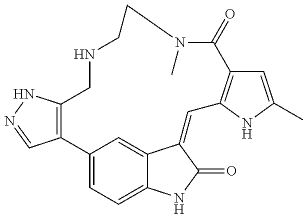 [SMILES: Cc1cc2c([nH]1)/C=C1\C(=O)Nc3ccc(cc31)-c1cn[nH]c1CNCCN(C)C2=O] | [3a(4)Z]-6,9-dimethyl-9,10,11,12,13,14- hexahydro-1,17- (ethanediylidene)pyrazolo[4,3- m]dipyrrolo[3,2-f:3′,4′- i][1,4]diazacyclopentadecine- 3,8(2H,5H)-dione |
| 57 | 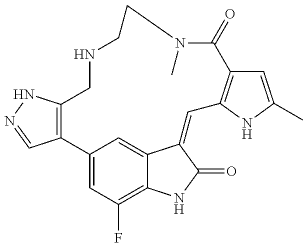 [SMILES: Cc1cc2c([nH]1)/C=C1\C(=O)Nc3c(F)cc(cc31)-c1cn[nH]c1CNCCN(C)C2=O] | [3a(4)Z]-20-fluoro-6,9-dimethyl- 9,10,11,12,13,14-hexahydro-1,17- (ethanediylidene)pyrazolo[4,3- m]dipyrrolo[3,2-f:3′,4′- i][1,4]diazacyclopentadecine- 3,8(2H,5H)-dione |
| 58 | 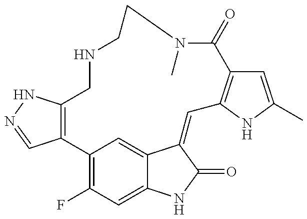 [SMILES: Cc1cc2c([nH]1)/C=C1\C(=O)Nc3cc(F)c(cc31)-c1cn[nH]c1CNCCN(C)C2=O] | [3a(4)Z]-19-fluoro-6,9-dimethyl- 9,10,11,12,13,14-hexahydro-1,17- (ethanediylidene)pyrazolo[4,3- m]dipyrrolo[3,2-f:3′,4′- i][1,4]diazacyclopentadecine- 3,8(2H,5H)-dione |
| 59 | 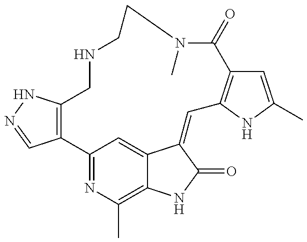 [SMILES: Cc1cc2c([nH]1)/C=C1\C(=O)Nc3c1cc(nc3C)-c1cn[nH]c1CNCCN(C)C2=O] | [3a(R)Z]-6,9,20-trimethyl- 9,10,11,12,13,14-hexahydro-17,1- (azenometheno)pyrazolo[4,3- m]dipyrrolo[3,2-f:3′,4′- i][1,4]diazacyclopentadecine- 3,8(2H,5H)-dione |
| 60 | 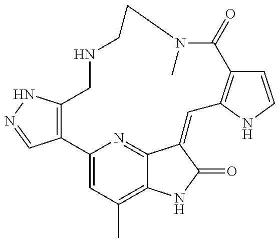 [SMILES: Cc1cc2nc3c1NC(=O)/C3=C\c1[nH]ccc1C(=O)N(C)CCNCc1[nH]ncc1-2] | [3a(4)Z]-9,20-dimethyl- 9,10,11,12,13,14-hexahydro-1,17- (ethanediylidene)pyrazolo[3,4- f]dipyrrolo[3,4-j:2′,3′- m][1,4,9]triazacyclopentadecine- 3,8(2H,5H)-dione |
| 61 | 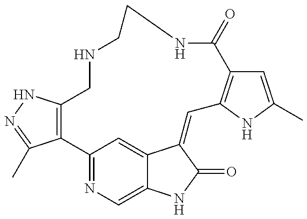 [SMILES: Cc1cc2c([nH]1)/C=C1\C(=O)Nc3cnc(cc31)-c1c(C)n[nH]c1CNCCNC2=O] | [3a(4)Z]-6,16-dimethyl- 9,10,11,12,13,14-hexahydro-17,1- (azenometheno)pyrazolo[4,3- m]dipyrrolo[3,2-f:3′,4′- i[1,4]diazacyclopentadecine- 3,8(2H,5H)-dione |
| 62 | 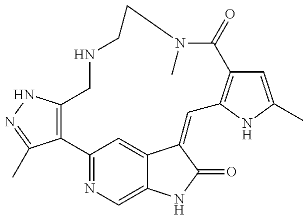 [SMILES: Cc1cc2c([nH]1)/C=C1\C(=O)Nc3cnc(cc31)-c1c(C)n[nH]c1CNCCN(C)C2=O] | [3a(4)Z]-6,9,16-trimethyl- 9,10,11,12,13,14-hexahydro-17,1- (azenometheno)pyrazolo[4,3- m]dipyrrolo[3,2-f:3′,4′- i][1,4]diazacyclopentadecine- 3,8(2H,5H)-dione |
| 63 | 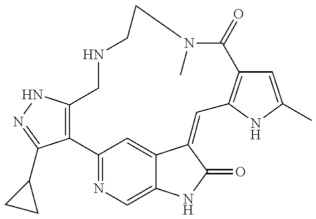 [SMILES: Cc1cc2c([nH]1)/C=C1\C(=O)Nc3cnc(cc31)-c1c(C3CC3)n[nH]c1CNCCN(C)C2=O] | [3a(4)Z]-16-cyclopropyl-6,9-dimethyl- 9,10,11,12,13,14-hexahydro-17,1- (azenometheno)pyrazolo[4,3- m]dipyrrolo[3,2-f:3′,4′- i][1,4]diazacyclopentadecine- 3,8(2H,5H)-dione |
| 64 | 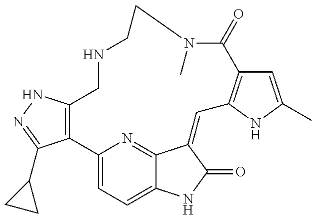 [SMILES: Cc1cc2c([nH]1)/C=C1\C(=O)Nc3ccc(nc31)-c1c(C3CC3)n[nH]c1CNCCN(C)C2=O] | [3a(4)Z]-16-cyclopropyl-6,9-dimethyl- 9,10,11,12,13,14-hexahydro-1,17- (ethanediylidene)pyrazolo[3,4- f]dipyrrolo[3,4-j:2′,3′- m][1,4,9]triazacyclopentadecine- 3,8(2H,5H)-dione |
| 65 | 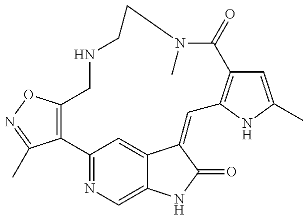 [SMILES: Cc1cc2c([nH]1)/C=C1\C(=O)Nc3cnc(cc31)-c1c(C)noc1CNCCN(C)C2=O] | [3a(4)Z]-6,9,16-trimethyl-10,11,12,13- tetrahydro-2H-17,1- (azenometheno)[1,2]oxazolo[4,5- m]dipyrrolo[3,2-f:3′,4′- i][1,4]diazacyclopentadecine- 3,8(5H,9H)-dione |
| 66 | 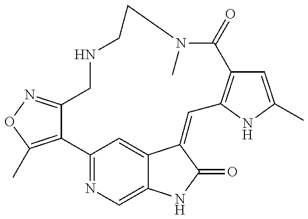 [SMILES: Cc1cc2c([nH]1)/C=C1\C(=O)Nc3cnc(cc31)-c1c(noc1C)CNCCN(C)C2=O] | [3a(4)Z]-6,9,16-trimethyl-10,11,12,13- tetrahydro-2H-17,1- (azenometheno)[1,2]oxazolo[4,3- m]dipyrrolo[3,2-f:3′,4′- i][1,4]diazacyclopentadecine- 3,8(5H,9H)-dione |
| 67 | 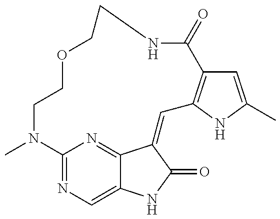 | [16a(17)Z]-2,11-dimethyl-6,7,10,11- tetrahydro-1H,9H-12,14- (azenometheno)dipyrrolo[3,4-g:2′,3′- j][1,4,6,13]oxatriazacyclopentadecine- 4,16(5H,15H)-dione |
| 68 | 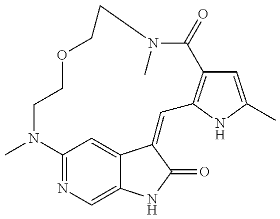 | [16a(17)Z]-2,5,11-trimethyl-6,7,10,11- tetrahydro-1H,9H-12,14- (azenometheno)dipyrrolo[3,2-f:3′,4′- i][1,4,13]oxadiazacyclopentadecine- 4,16(5H,15H)-dione |
| 69 | 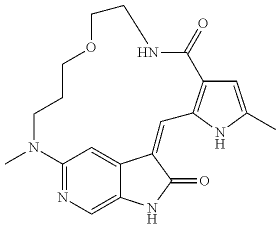 | [17a(18)Z]-2,12-dimethyl- 6,7,9,10,11,12-hexahydro-1H-13,15- (azenometheno)dipyrrolo[3,2-f:3′,4′- i][1,4,13]oxadiazacyclohexadecine- 4,17(5H,16H)-dione |
| 70 | 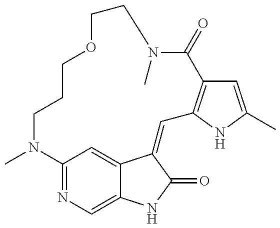 | [17a(18)Z]-2,5,12-trimethyl- 6,7,9,10,11,12-hexahydro-1H-13,15- (azenometheno)dipyrrolo[3,2-f:3′,4′- i][1,4,13]oxadiazacyclohexadecine- 4,17(5H,16H)-dione |
| 71 | 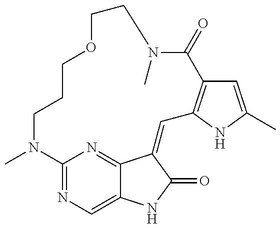 | [17a(18)Z]-2,5,12-trimethyl- 6,7,9,10,11,12-hexahydro-1H-13,15- (azenometheno)dipyrrolo[3,2-f:3′,4′- i][1,4,11,13]oxatriazacyclohexadecine- 4,17(5H,16H)-dione |
| 72 | 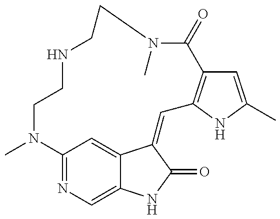 | [16a(17)Z]-2,5,11-trimethyl- 6,7,8,9,10,11-hexahydro-1H-12,14- (azenometheno)dipyrrolo[3,2-i:3′,4′- l][1,4,7]triazacyclopentadecine- 4,16(5H,15H)-dione |
| 73 | 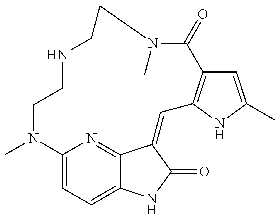 | [16a(17)Z]-2,5,11-trimethyl- 6,7,8,9,10,11-hexahydro-1H-12,14- (ethanediylidene)dipyrrolo[3,2-k:3′,4′- n][1,3,6,9]tetraazacyclopentadecine- 4,16(5H,15H)-dione |
| 74 | 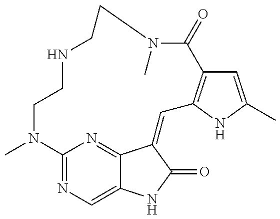 | [16a(17)Z]-2,5,11-trimethyl- 6,7,8,9,10,11-hexahydro-1H-12,14- (azenometheno)dipyrrolo[3,2-k:3′,4′- n][1,3,6,9]tetraazacyclopentadecine- 4,16(5H,15H)-dione |
| 75 | 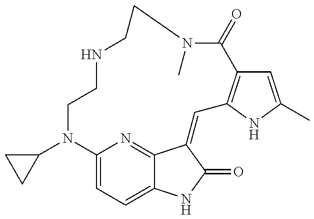 [SMILES: Cc1cc2c([nH]1)/C=C1\C(=O)Nc3ccc(nc31)N(C1CC1)CCNCCN(C)C2=O] | [16a(17)Z]-11-cyclopropyl-2,5- dimethyl-6,7,8,9,10,11-hexahydro-1H- 12,14-(ethanediylidene)dipyrrolo[3,2- k:3′,4′- n][1,3,6,9]tetraazacyclopentadecine- 4,16(5H,15H)-dione |
| 76 | 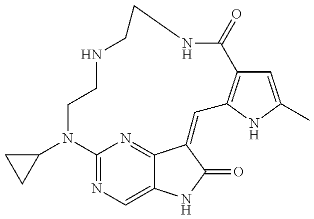 [SMILES: Cc1cc2c([nH]1)/C=C1\C(=O)Nc3cnc(nc31)N(C1CC1)CCNCCNC2=O] | [16a(17)Z]-11-cyclopropyl-2-methyl- 6,7,8,9,10,11-hexahydro-1H-12,14- (azenometheno)dipyrrolo[3,2-k:3′,4′- n][1,3,6,9]tetraazacyclopentadecine- 4,16(5H,1H)-dione |
| 77 | 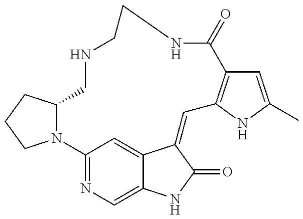 [SMILES: Cc1cc2c([nH]1)/C=C1\C(=O)Nc3cnc(cc31)N1CCC[C@@H]1CNCCNC2=O] | [3a(4)Z,13aR]-6-methyl- 10,11,12,13,13a,14,15,16-octahydro- 2H-18,1-(azenometheno)tripyrrolo[1,2- a:3′,2′-i:3″,4″- l][1,4,7]triazacyclopentadecine- 3,8(5H,9H)-dione |
| 78 | 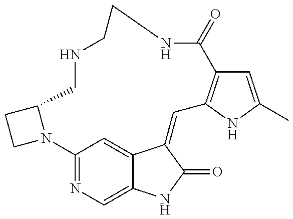 [SMILES: Cc1cc2c([nH]1)/C=C1\C(=O)Nc3cnc(cc31)N1CC[C@@H]1CNCCNC2=O] | [3a(4)Z,13aR)-6-methyl- 9,10,11,12,13,13a,14,15-octahydro- 17,1-(azenometheno)azeno[1,2- a]dipyrrolo[3,2-i:3′,4′- l][1,4,7]triazacyclopentadecine- 3,8(2H,5H)-dione |
| 79 | 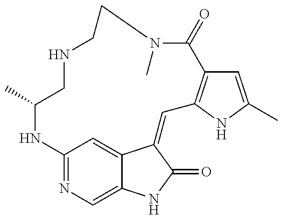 | [10R,16a(17)Z]-2,5,10-trimethyl- 6,7,8,9,10,11-hexahydro-1H-12,14- (azenometheno)dipyrrolo[3,2-i:3′,4′- l][1,4,7]triazacyclopentadecine- 4,16(5H,15H)-dione |
| 80 | 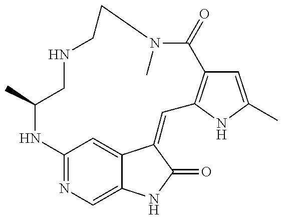 | [10S,16a(17)Z]-2,5,10-trimethyl- 6,7,8,9,10,11-hexahydro-1H-12,14- (azenometheno)dipyrrolo[3,2-i:3′,4′- l][1,4,7]triazacyclopentadecine- 4,16(5H,15H)-dione |
| 81 | 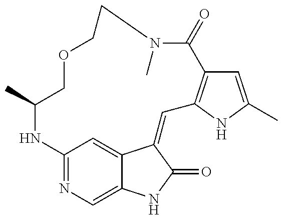 | [10S,16a(17)Z]-2,5,10-trimethyl- 6,7,10,11-tetrahydro-1H,9H-12,14- (azenometheno)dipyrrolo[3,2-f:3′,4′- i][1,4,13]oxadiazacyclopentadecine- 4,16(5H,15H)-dione |
| 82 | 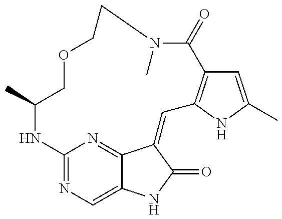 | [10S,16a(17)Z]-2,5,10-trimethyl- 6,7,10,11-tetrahydro-1H,9H-12,14- (azenometheno)dipyrrolo[3,4-g:2′,3′- j][1,4,6,13]oxatriazacyclopentadecine- 4,16(5H,15H)-dione |
| 83 | 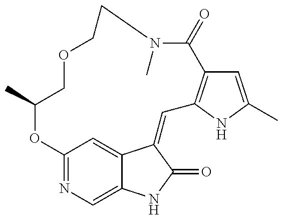 | [10S,16a(17)Z]-2,5,10-trimethyl- 6,7,9,10-tetrahydro-1H-12,14- (azenometheno)dipyrrolo[3,2-i:3′,4′- l][1,4,7]dioxazacyclopentadecine- 4,16(5H,15H)-dione |
| 84 | 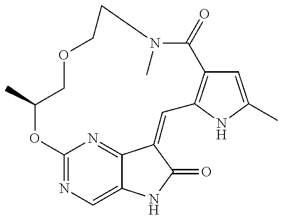 | [10S,16a(17)Z]-2,5,10-trimethyl- 6,7,9,10-tetrahydro-1H-12,14- (azenometheno)dipyrrolo[3,4-d:2′,3′- g][1,13,3,10]dioxadiazacyclopentadecine- 4,16(5H,15H)-dione |
| 85 | 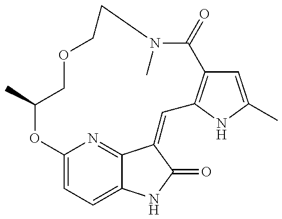 | [10S,16a(17)Z]-2,5,10-trimethyl- 6,7,9,10-tetrahydro-1H-12,14- (ethanediylidene)dipyrrolo[3,4-d:2′,3′- g][1,13,3,10]dioxadiazacyclopentadecine- 4,16(5H,15H)-dione |
| 86 | 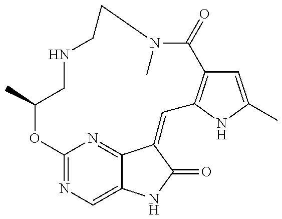 | [10S,16a(17)Z]-2,5,10-trimethyl- 5,6,7,8,9,10-hexahydro-12,14- (azenometheno)dipyrrolo[3,4-d:2′,3′- g][1,3,10,13]oxatriazacyclopentadecine- 4,16(1H,15H)-dione |
| 87 | 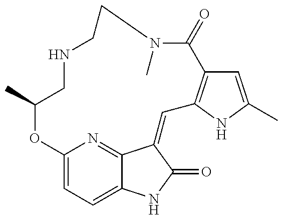 | [10S,16a(17)Z]-2,5,10-trimethyl- 5,6,7,8,9,10-hexahydro-12,14- (ethanediylidene)dipyrrolo[3,4-d:2′,3′- g][1,3,10,13]oxatriazacyclopentadecine- 4,16(1H,15H)-dione |
| 88 | 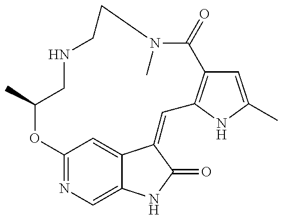 | [10S,16a(17)Z]-2,5,10-trimethyl- 5,6,7,8,9,10-hexahydro-12,14- (azenometheno)dipyrrolo[3,2-i:3′,4′- l][1,4,7]oxadiazacyclopentadecine- 4,16(1H,15H)-dione |
| 89 | 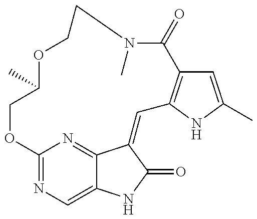 [SMILES: Cc1cc2c([nH]1)/C=C1\C(=O)Nc3cnc(nc31)OC[C@@H](C)OCCN(C)C2=O] | [9R,16a(17)Z]-2,5,9-trimethyl-6,7,9,10- tetrahydro-1H-12,14- (azenometheno)dipyrrolo[3,4-d:2′,3′- g][1,13,3,10]dioxadiazacyclopentadecine- 4,16(5H,15H)-dione |
| 90 | 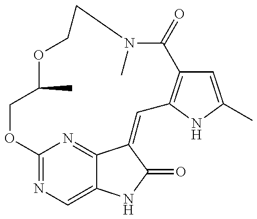 [SMILES: Cc1cc2c([nH]1)/C=C1\C(=O)Nc3cnc(nc31)OC[C@H](C)OCCN(C)C2=O] | [9S,16a(17)Z]-2,5,9-trimethyl-6,7,9,10- tetrahydro-1H-12,14- (azenometheno)dipyrrolo[3,4-d:2′,3′- g][1,13,3,10]dioxadiazacyclopentadecine- 4,16(5H,15H)-dione |
| 91 | 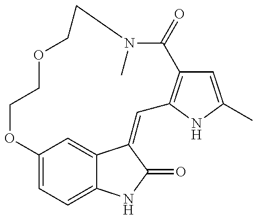 [SMILES: Cc1cc2c([nH]1)/C=C1\C(=O)Nc3ccc(cc31)OCCOCCN(C)C2=O] | [16a(17)Z]-2,5-dimethyl-6,7,9,10- tetrahydro-1H-12,14- (ethanediylidene)dipyrrolo[3,2-i:3′,4′- l][1,4,7]dioxazacyclopentadecine- 4,16(5H,15H)-dione |
| 92 | 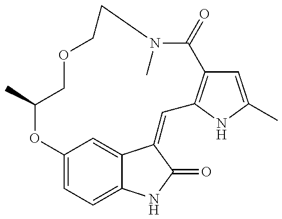 | [10S,16a(17)Z]-2,5,10-trimethyl- 6,7,9,10-tetrahydro-1H-12,14- (ethanediylidene)dipyrrolo[3,2-i:3′,4′- l][1,4,7]dioxazacyclopentadecine- 4,16(5H,15H)-dione |
| 93 | 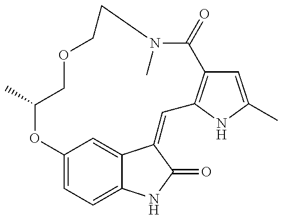 | [10R,16a(17)Z]-2,5,10-trimethyl- 6,7,9,10-tetrahydro-1H-12,14- (ethanediylidene)dipyrrolo[3,2-i:3′,4′- l][1,4,7]dioxazacyclopentadecine- 4,16(5H,15H)-dione |
| 94 | 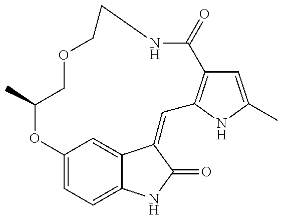 | [10S,16a(17)Z]-2,10-dimethyl-6,7,9,10- tetrahydro-1H-12,14- (ethanediylidene)dipyrrolo[3,2-i:3′,4′- l][1,4,7]dioxazacyclopentadecine- 4,16(5H,15H)-dione |
| 95 | 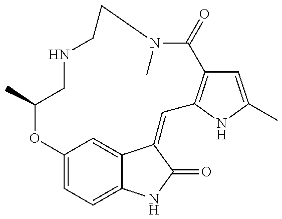 | [10S,16a(17)Z]-2,5,10-trimethyl- 5,6,7,8,9,10-hexahydro-12,14- (ethanediylidene)dipyrrolo[3,2-i:3′,4′- l][1,4,7]oxadiazacyclopentadecine- 4,16(1H,15H)-dione |
| 96 | 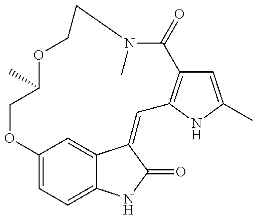 [SMILES: Cc1cc2c([nH]1)/C=C1\C(=O)Nc3ccc(cc31)OC[C@@H](C)OCCN(C)C2=O] | [9R,16a(17)Z]-2,5,9-trimethyl-6,7,9,10- tetrahydro-1H-12,14- (ethanediylidene)dipyrrolo[3,2-i:3′,4′- l][1,4,7]dioxazacyclopentadecine- 4,16(5H,15H)-dione |
| 97 | 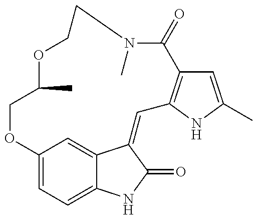 [SMILES: Cc1cc2c([nH]1)/C=C1\C(=O)Nc3ccc(cc31)OC[C@H](C)OCCN(C)C2=O] | [9S,16a(17)Z]-2,5,9-trimethyl-6,7,9,10- tetrahydro-1H-12,14- (ethanediylidene)dipyrrolo[3,2-i:3′,4′- l][1,4,7]dioxazacyclopentadecine- 4,16(5H,15H)-dione |
| 98 | 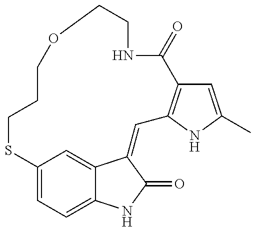 [SMILES: Cc1cc2c([nH]1)/C=C1\C(=O)Nc3ccc(cc31)SCCCOCCNC2=O] | [17a(18)Z]-2-methyl-6,7,10,11- tetrahydro-1H,9H-13,15- (ethanediylidene)dipyrrolo[3,2-f:3′,4′- i][1,13,4]oxathiazacyclohexadecine- 4,17(5H,16H)-dione |
| 99 | 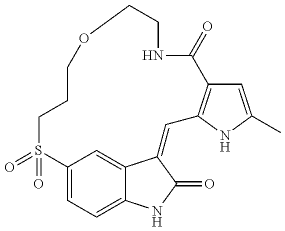 [SMILES: Cc1cc2c([nH]1)/C=C1\C(=O)Nc3ccc(cc31)S(=O)(=O)CCCOCCNC2=O] | [17a(18)Z]-2-methyl-6,7,10,11- tetrahydro-1H-13,15-(ethanediylidene)- 12λ⁶-dipyrrolo[3,2-f:3′,4′- i][1,13,4]oxathiazacyclohexadecine- 4,12,12,17(5H,9H,16H)-tetrone |
| 100 | 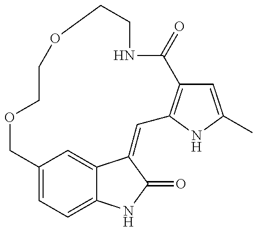 [SMILES: Cc1cc2c([nH]1)/C=C1\C(=O)Nc3ccc(cc31)COCCOCCNC2=O] | [17a(18)Z]-2-methyl-6,7,9,10- tetrahydro-1H,12H-13,15- (ethanediylidene)dipyrrolo[3,2-i:3′,4′- l][1,4,7]dioxazacyclohexadecine- 4,17(5H,16H)-dione |
| 101 | 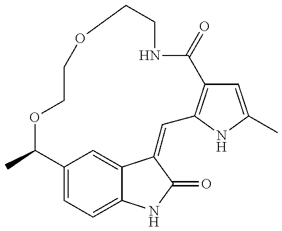 | [12R,17a(18)Z]-2,12-dimethyl-6,7,9,10- tetrahydro-1H,12H-13,15- (ethanediylidene)dipyrrolo[3,2-i:3′,4′- l][1,4,7]dioxazacyclohexadecine- 4,17(5H,16H)-dione |
| 102 | 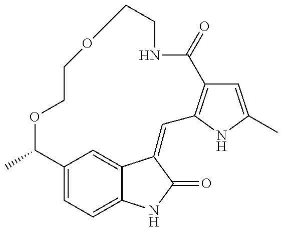 | [12S,17a(18)Z]-2,12-dimethyl-6,7,9,10- tetrahydro-1H,12H-13,15- (ethanediylidene)dipyrrolo[3,2-i:3′,4′- l][1,4,7]dioxazacyclohexadecine- 4,17(5H,16H)-dione |
| 103 | 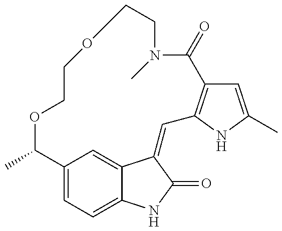 | [12S,17a(18)Z]-2,5,12-trimethyl- 6,7,9,10-tetrahydro-1H,12H-13,15- (ethanediylidene)dipyrrolo[3,2-i:3′,4′- l][1,4,7]dioxazacyclohexadecine- 4,17(5H,16H)-dione |
| 104 | 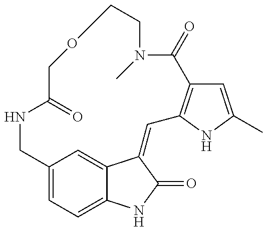 [SMILES: Cc1cc2c([nH]1)/C=C1\C(=O)Nc3ccc(cc31)CNC(=O)COCCN(C)C2=O] | [17a(18)Z]-2,5-dimethyl-6,7,11,12- tetrahydro-1H-13,14- (ethanediylidene)dipyrrolo[3,2-f:3′,4′- i][1,4,14]oxadiazacyclohexadecine- 4,10,17(5H,9H,16H)-trione |
| 105 | 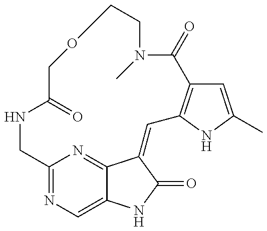 [SMILES: Cc1cc2c([nH]1)/C=C1\C(=O)Nc3cnc(nc31)CNC(=O)COCCN(C)C2=O] | [17a(18)Z]-2,5-dimethyl-6,7,11,12- tetrahydro-1H-13,15- (azenometheno)dipyrrolo[3,4-h:2′,3′- k][1,4,7,14]oxatriazacyclohexadecine- 4,10,17(5H,9H,16H)-trione |
| 106 | 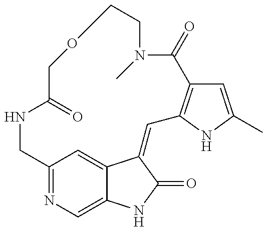 [SMILES: Cc1cc2c([nH]1)/C=C1\C(=O)Nc3cnc(cc31)CNC(=O)COCCN(C)C2=O] | [17a(18)Z]-2,5-dimethyl-6,7,11,12- tetrahydro-1H-13,15- (azenometheno)dipyrrolo[3,2-f:3′,4′- i][1,4,14]oxadiazacyclohexadecine- 4,10,17(5H,9H,16H)-trione |
| 107 | 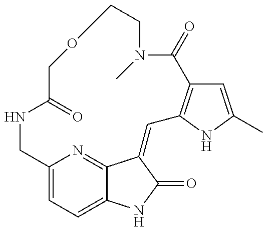 [SMILES: Cc1cc2c([nH]1)/C=C1\C(=O)Nc3ccc(nc31)CNC(=O)COCCN(C)C2=O] | [17a(18)Z]-2,5-dimethyl-6,7,11,12- tetrahydro-1H-13,15- (ethanediylidene)dipyrrolo[3,4-h:2′,3′- k][1,4,7,14]oxatriazacyclohexadecine- 4,10,17(5H,9H,16H)-trione |
| 108 | 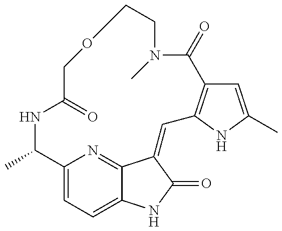 | [12S,17a(18)Z]-2,5,12-trimethyl- 6,7,11,12-tetrahydro-1H-13,15- (ethanediylidene)dipyrrolo[3,4-h:2′,3′- k][1,4,7,14]oxatriazacyclohexadecine- 4,10,17(5H,9H,16H)-trione |
| 109 | 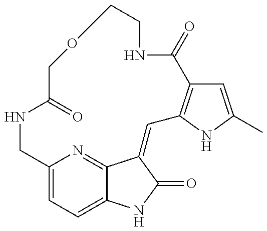 [SMILES: Cc1cc2c([nH]1)/C=C1\C(=O)Nc3ccc(nc31)CNC(=O)COCCNC2=O] | [17a(18)Z]-2-methyl-6,7,11,12- tetrahydro-1H-13,15- (ethanediylidene)dipyrrolo[3,4-h:2′,3′- k][1,4,7,14]oxatriazacyclohexadecine- 4,10,17(5H,9H,16H)-trione |
| 110 | 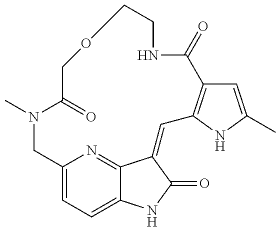 | [17a(18)Z]-2,11-dimethyl-6,7,11,12- tetrahydro-1H-13,15- (ethanediylidene)dipyrrolo[3,4-h:2′,3′- k][1,4,7,14]oxatriazacyclohexadecine- 4,10,17(5H,9H,16H)-trione |
| 111 | 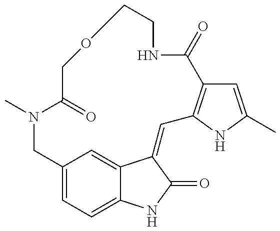 | [17a(18)Z]-2,11-dimethyl-6,7,11,12- tetrahydro-1H-13,15- (ethanediylidene)dipyrrolo[3,2-f:3′,4′- i][1,4,14]oxadiazacyclohexadecine- 4,10,17(5H,9H,16H)-trione |
| 112 | 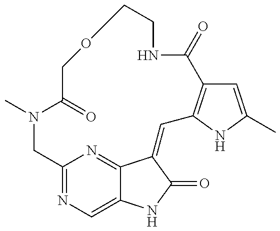 | [17a(18)Z]-2,11-dimethyl-6,7,11,12- tetrahydro-1H-13,15- (azenometheno)dipyrrolo[3,4-h:2′,3′- k][1,4,7,14]oxatriazacyclohexadecine- 4,10,17(5H,9H,16H)-trione |
| 113 | 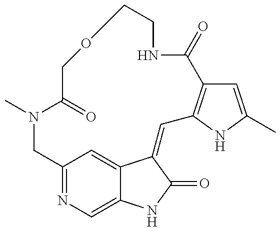 | [17a(18)Z]-2,11-dimethyl-6,7,11,12- tetrahydro-1H-13,15- (azenometheno)dipyrrolo[3,2-f:3′,4′- i][1,4,14]oxadiazacyclohexadecine- 4,10,17(5H,9H,16H)-trione |
| 114 | 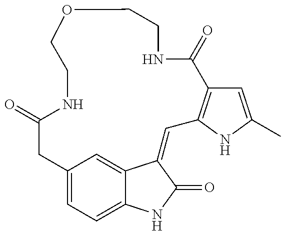 [SMILES: Cc1cc2c([nH]1)/C=C1\C(=O)Nc3ccc(cc31)CC(=O)NCCOCCNC2=O] | [18a(19)Z]-2-methyl-6,7,10,11- tetrahydro-1H,9H-14,16- (ethanediylidene)dipyrrolo[3,2-f:3′,4′- i][1,4,15]oxadiazacycloheptadecine- 4,12,18(5H,13H,17H)-trione |
| 115 | 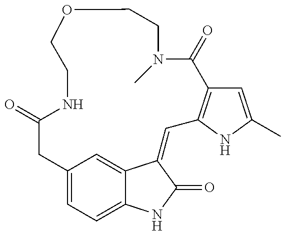 [SMILES: Cc1cc2c([nH]1)/C=C1\C(=O)Nc3ccc(cc31)CC(=O)NCCOCCN(C)C2=O] | [18a(19)Z]-2,5-dimethyl-6,7,10,11- tetrahydro-1H,9H-14,16- (ethanediylidene)dipyrrolo[3,2-f:3′,4′- i][1,4,15]oxadiazacycloheptadecine- 4,12,18(5H,13H,17H)-trione |
| 116 | 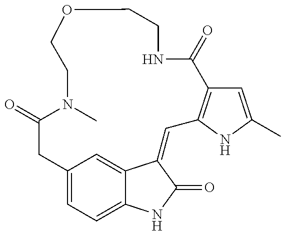 [SMILES: Cc1cc2c([nH]1)/C=C1\C(=O)Nc3ccc(cc31)CC(=O)N(C)CCOCCNC2=O] | [18a(19)Z]-2,11-dimethyl-6,7,10,11- tetrahydro-1H,9H-14,16- (ethanediylidene)dipyrrolo[3,2-f:3′,4′- i][1,4,15]oxadiazacycloheptadecine- 4,12,18(5H,13H,17H)-trione |
| 117 | 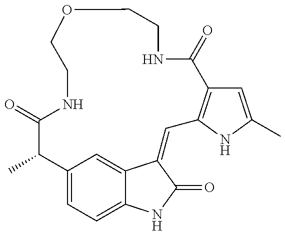 [SMILES: Cc1cc2c([nH]1)/C=C1\C(=O)Nc3ccc(cc31)[C@H](C)C(=O)NCCOCCNC2=O] | [13S,18a(19)Z]-2,13-dimethyl- 6,7,10,11-tetrahydro-1H,9H-14,16- (ethanediylidene)dipyrrolo[3,2-f:3′,4′- i][1,4,15]oxadiazacycloheptadecine- 4,12,18(5H,13H,17H)-trione |
| 118 | 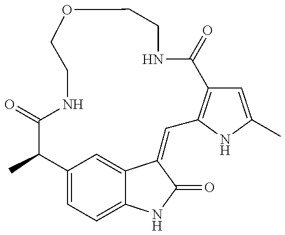 [SMILES: Cc1cc2c([nH]1)/C=C1\C(=O)Nc3ccc(cc31)[C@@H](C)C(=O)NCCOCCNC2=O] | [13R,18a(19)Z]-2,13-dimethyl- 6,7,10,11-tetrahydro-1H,9H-14,16- (ethanediylidene)dipyrrolo[3,2-f:3′,4′- i][1,4,15]oxadiazacycloheptadecine- 4,12,18(5H,13H,17H)-trione |
| 119 | 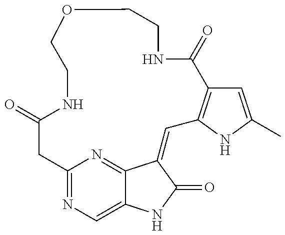 [SMILES: Cc1cc2c([nH]1)/C=C1\C(=O)Nc3cnc(nc31)CC(=O)NCCOCCNC2=O] | [18a(19)Z]-2-methyl-6,7,10,11- tetrahydro-1H,9H-14,16- (azenometheno)dipyrrolo[3,4-i:2′,3′- l][1,4,8,15]oxatriazacycloheptadecine- 4,12,18(5H,13H,17H)-trione |
| 120 | 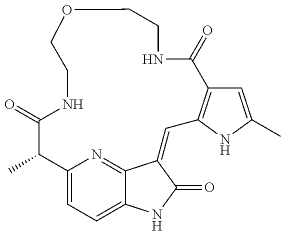 [SMILES: Cc1cc2c([nH]1)/C=C1\C(=O)Nc3ccc(nc31)[C@H](C)C(=O)NCCOCCNC2=O] | [13S,18a(19)Z]-2,13-dimethyl- 6,7,10,11-tetrahydro-1H,9H-14,16- (ethanediylidene)dipyrrolo[3,4-i:2′,3′- l][1,4,8,15]oxatriazacycloheptadecine- 4,12,18(5H,13H,17H)-trione |
| 121 | 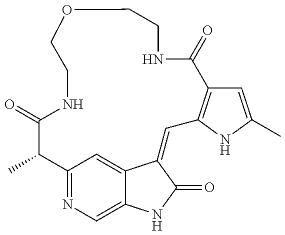 [SMILES: Cc1cc2c([nH]1)/C=C1\C(=O)Nc3cnc(cc31)[C@H](C)C(=O)NCCOCCNC2=O] | [13S,18a(19)Z]-2,13-dimethyl- 6,7,10,11-tetrahydro-1H,9H-14,16- (azenometheno)dipyrrolo[3,2-f:3′,4′- i][1,4,15]oxadiazacycloheptadecine- 4,12,18(5H,13H,17H)-trione |
| 122 | 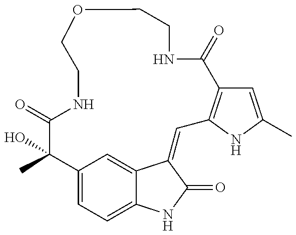 [SMILES: Cc1cc2c([nH]1)/C=C1\C(=O)Nc3ccc(cc31)[C@](C)(O)C(=O)NCCOCCNC2=O] | [13S,18a(19)Z]-2,13-hydroxy-2,13- dimethyl-6,7,10,11-tetrahydro-1H,9H- 14,16-(ethanediylidene)dipyrrolo[3,2- f:3′,4′- i][1,4,15]oxadiazacycloheptadecine- 4,12,18(5H,13H,17H)-trione |
| 123 | 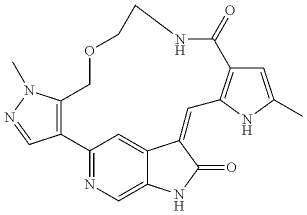 [SMILES: Cc1cc2c([nH]1)/C=C1\C(=O)Nc3cnc(cc31)-c1cnn(C)c1COCCNC2=O] | [3a(4)Z]-6,14-dimethyl-10,11,13,14- tetrahydro-2H-17,1- (azenometheno)pyrazolo[4,3- m]dipyrrolo[3,2-f:3′,4′- i][1,4]oxazacyclopentadecine- 3,8(5H,9H)-dione |
| 124 | 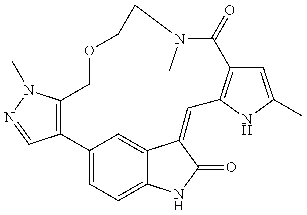 [SMILES: Cc1cc2c([nH]1)/C=C1\C(=O)Nc3ccc(cc31)-c1cnn(C)c1COCCN(C)C2=O] | [3a(4)Z]-6,9,14-trimethyl-10,11,13,14- tetrahydro-2H-1,17- (ethanediylidene)pyrazolo[4,3- m]dipyrrolo[3,2-f:3′,4′- i][1,4]oxazacyclopentadecine- 3,8(5H,9H)-dione |
| 125 | 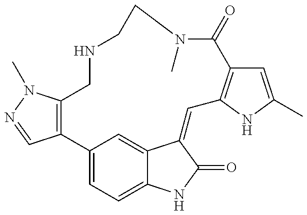 [SMILES: Cc1cc2c([nH]1)/C=C1\C(=O)Nc3ccc(cc31)-c1cnn(C)c1CNCCN(C)C2=O] | [3a(4)Z]-6,9,14-trimethyl- 9,10,11,12,13,14-hexahydro-1,17- (ethanediylidene)pyrazolo[4,3- m]dipyrrolo[3,2-f:3′,4′- i][1,4]diazacyclopentadecine- 3,8(2H,5H)-dione |
| 126 | 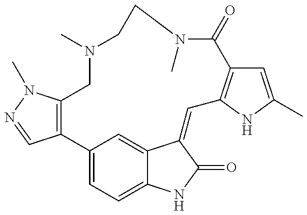 [SMILES: Cc1cc2c([nH]1)/C=C1\C(=O)Nc3ccc(cc31)-c1cnn(C)c1CN(C)CCN(C)C2=O] | [3a(4)Z]-6,9,12,14-tetramethyl- 9,10,11,12,13,14-hexahydro-1,17- (ethanediylidene)pyrazolo[4,3- m]dipyrrolo[3,2-f:3′,4′- i][1,4]diazacyclopentadecine- 3,8(2H,5H)-dione |
| 127 | 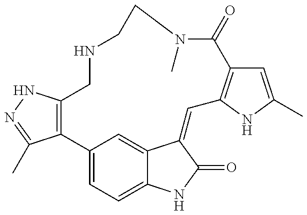 [SMILES: Cc1cc2c([nH]1)/C=C1\C(=O)Nc3ccc(cc31)-c1c(C)n[nH]c1CNCCN(C)C2=O] | [3a(4)Z]-6,9,16-trimethyl- 9,10,11,12,13,14-hexahydro-1,17- (ethanediylidene)pyrazolo[4,3- m]dipyrrolo[3,2-f:3′,4′- i][1,4]diazacyclopentadecine- 3,8(2H,5H)-dione |
| 128 | 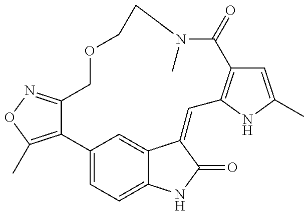 [SMILES: Cc1cc2c([nH]1)/C=C1\C(=O)Nc3ccc(cc31)-c1c(noc1C)COCCN(C)C2=O] | [3a(4)Z]-6,9,16-trimethyl-1,11- dihydro-2H,13H-1,17- (ethanediylidene)[1,4]oxazolo[4,3- m]dipyrrolo[3,2-f:3′,4′- i][1,4]oxazacyclopentadecine- 3,8(5H,9H)-dione |
| 129 | 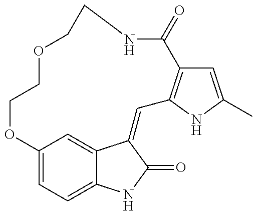 [SMILES: Cc1cc2c([nH]1)/C=C1\C(=O)Nc3ccc(cc31)OCCOCCNC2=O] | [16a(17)Z]-2-methyl-6,7,9,10- tetrahydro-1H-12,14- (ethanediylidene)dipyrrolo[3,2-i:3′,4′- l][1,4,7]dioxazacyclopentadecine- 4,16(5H,15H)-dione |
| 130 | 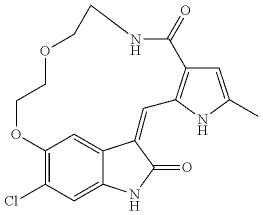 [SMILES: Cc1cc2c([nH]1)/C=C1\C(=O)Nc3cc(Cl)c(cc31)OCCOCCNC2=O] | [16a(17)Z]-19-chloro-2-methyl- 6,7,9,10-tetrahydro-1H-12,14- (ethanediylidene)dipyrrolo[3,2-i:3′,4′- l][1,4,7]dioxazacyclopentadecine- 4,16(5H,15H)-dione |
| 131 | 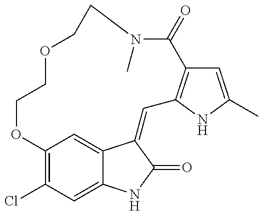 [SMILES: Cc1cc2c([nH]1)/C=C1\C(=O)Nc3cc(Cl)c(cc31)OCCOCCN(C)C2=O] | [16a(17)Z]-19-chloro-2,5-dimethyl- 6,7,9,10-tetrahydro-1H-12,14- (ethanediylidene)dipyrrolo[3,2-i:3′,4′- l][1,4,7]dioxazacyclopentadecine- 4,16(5H,15H)-dione |
| 132 | 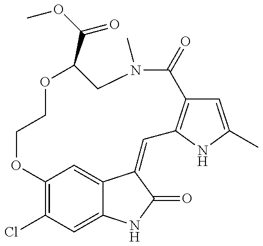 [SMILES: COC(=O)[C@H]1CN(C)C(=O)c2cc(C)[nH]c2/C=C2\C(=O)Nc3cc(Cl)c(cc32)OCCO1] | Methyl [7R,16a(17)Z]-19-chloro-2,5- dimethyl-4,16-dioxo- 4,5,6,7,9,10,15,16-octahydro-1H- 12,14-(ethanediylidene)dipyrrolo[3,2- i:3′,4′- l][1,4,7]dioxazacyclopentadecine-7- carboxaylte |
| 133 | 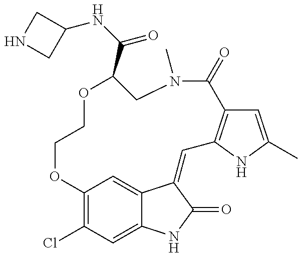 [SMILES: Cc1cc2c([nH]1)/C=C1\C(=O)Nc3cc(Cl)c(cc31)OCCO[C@@H](C(=O)NC1CNC1)CN(C)C2=O] | [7R,16a(17)Z]-N-(azetidin-3-yl)-19- chloro-2,5-dimethyl-4,16-dioxo- 4,5,6,7,9,10,15,16-octahydro-1H- 12,14-(ethanediylidene)dipyrrolo[3,2- i:3′,4′- l][1,4,7]dioxazacyclopentadecine-7- carboxamide |
| 134 | 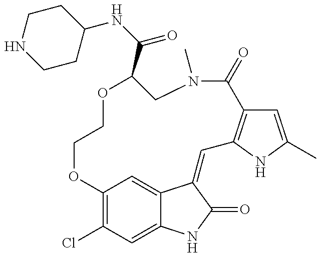 [SMILES: Cc1cc2c([nH]1)/C=C1\C(=O)Nc3cc(Cl)c(cc31)OCCO[C@@H](C(=O)NC1CCNCC1)CN(C)C2=O] | [7R,16a(17)Z]-19-chloro-2,5- dimethyl-4,16-dioxo-N-(piperidin-4- yl)-4,5,6,7,9,10,15,16-octahydro-1H- 12,14-(ethaneidylidene)dipyrrolo[3,2- i:3′,4′- l][1,4,7]dioxazacyclopentadecine-7- carboxamide |
| 135 | 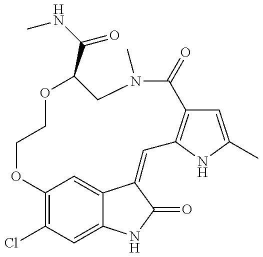 [SMILES: CNC(=O)[C@H]1CN(C)C(=O)c2cc(C)[nH]c2/C=C2\C(=O)Nc3cc(Cl)c(cc32)OCCO1] | [7R,16a(17)Z]-19-chloro-N,2,5- trimethyl-4,16-dioxo- 4,5,6,7,9,10,15,16-octahydro-1H- 12,14-(ethanediylidene)dipyrrolo[3,2- i:3′,4′- l][1,4,7]dioxazacyclopentadecine-7- carboxamide |
| 136 | 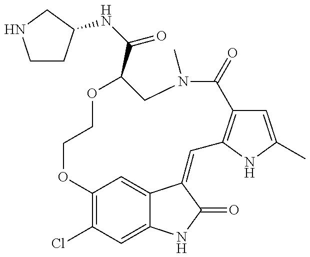 [SMILES: Cc1cc2c([nH]1)/C=C1\C(=O)Nc3cc(Cl)c(cc31)OCCO[C@@H](C(=O)N[C@@H]1CCNC1)CN(C)C2=O] | [7R,16a(17)Z]-19-chloro-2,5- dimethyl-4,16-dioxo-N-[(3R)- pyrrolidin-3-yl]-4,5,6,7,9,10,15,16- octahydro-1H-12,14- (ethanediylidene)dipyrrolo[3,2-i:3′,4′- l][1,4,7]dioxazacyclopentadecine-7- carboxamide |
| 137 | 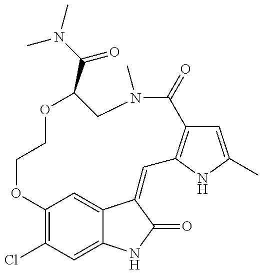 [SMILES: Cc1cc2c([nH]1)/C=C1\C(=O)Nc3cc(Cl)c(cc31)OCCO[C@@H](C(=O)N(C)C)CN(C)C2=O] | [7R,16a(17)Z]-19-chloro-N,N,2,5- tetramethyl-4,16-dioxo- 4,5,6,7,9,10,15,16-octahydro-1H- 12,14-(ethanediylidene)dipyrrolo[3,2- i:3′,4′- l][1,4,7]dioxazacyclopentadecine-7- carboxamide |
| 138 | 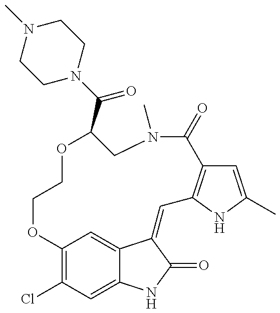 | [7R,16a(17)Z]-19-chloro-2,5- dimethyl-7-(4-methylpipearazine-1- carbonyl)-6,7,9,10-tetrahydro-1H- 12,14-(ethanediylidene)dipyrrolo[3,2- i:3′,4′- l][1,4,7]dioxazacyclopentadecine- 4,16(5H,15H)-dione |
| 139 | 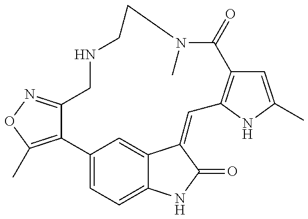 [SMILES: Cc1cc2c([nH]1)/C=C1\C(=O)Nc3ccc(cc31)-c1c(noc1C)CNCCN(C)C2=O] | [3a(4)Z]-6,9,16-trimethyl- 10,11,12,13-tetrahydro-2H-1,17- (ethanediylidene)[1,2]oxazolo[4,3- m]dipyrrolo[3,2-f:3′,4′- i][1,4]diazacyclopentadecine- 3,8(5H,9H)-dione |
| 92 | 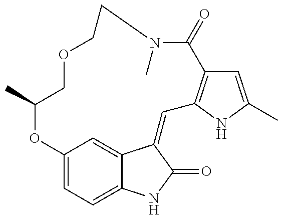 | [10S,16a(17)Z]-2,5,10-trimethyl- 6,7,9,10-tetrahydro-1H-12,14- (ethenediylidene)dipyrrolo[3,2-i:3′,4′- l][1,4,7]dioxazacyclopentadecine- 4,16(5H,15H)-dione |
| 140 | 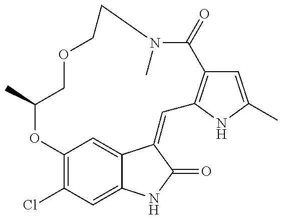 | [10S,16a(17)Z]-19-chloro-2,5,10- trimethyl-6,7,9,10-tetrahydro-1H- 12,14-(ethanediylidene)dipyrrolo[3,2- i:3′,4′- l][1,4,7]dioxazacyclopentadecine- 4,16(5H,15H)-dione |
| 140 | 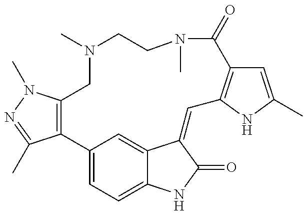 [SMILES: Cc1cc2c([nH]1)/C=C1\C(=O)Nc3ccc(cc31)-c1c(C)nn(C)c1CN(C)CCN(C)C2=O] | [3a(4)Z]-6,9,12,14,16-pentamethyl- 9,10,11,12,13,14-hexahydro-1,17- (ethanediylidene)pyrazolo[4,3- m]dipyrrolo[3,2-f:3′,4′- i][1,4]diazacyclopentadecine- 3,8(2H,5H)-dione |
| 141 | 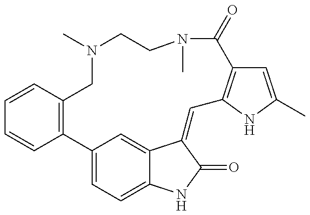 [SMILES: Cc1cc2c([nH]1)/C=C1\C(=O)Nc3ccc(cc31)-c1ccccc1CN(C)CCN(C)C2=O] | [3a(4)Z]-6,9,12-trimethyl- 10,11,12,13-tetrahydro-2H-1,18- (ethanediylidene)dipyrrolo[3,2- g:3′,4′- j][2,5]benzodiazacyclopentadecine- 3,8(5H,9H)-dione |
| 142 | 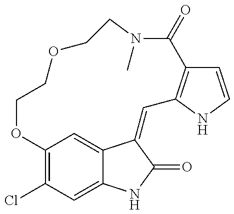 [SMILES: CN1CCOCCOc2cc3c(cc2Cl)NC(=O)/C3=C\c2[nH]ccc2C1=O] | [16a(17)Z]-19-chloro-5-methyl- 6,7,9,10-tetrahydro-1H-12,14- (ethanediylidene)dipyrrolo[3,2-i:3′,4′- l[1,4,7]dioxazacyclopentadecine- 4,16(5H,15H)-dione |
| 143 | 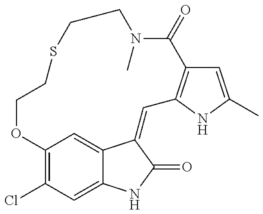 [SMILES: Cc1cc2c([nH]1)/C=C1\C(=O)Nc3cc(Cl)c(cc31)OCCSCCN(C)C2=O] | [16a(17)Z]-19-chloro-2,5-dimethyl- 6,7,9,10-tetrahydro-1H-12,14- (ethanediylidene)dipyrrolo[3,2-i:3′,4′- l][1,4,7]oxathiazacyclopentadecine- 4,16(5H,15H)-dione |
| 144 | 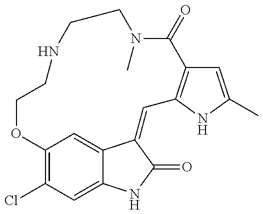 [SMILES: Cc1cc2c([nH]1)/C=C1\C(=O)Nc3cc(Cl)c(cc31)OCCNCCN(C)C2=O] | [16a(17)Z]-19-chloro-2,5-dimethyl- 5,6,7,8,9,10-hexahydro-12,14- (ethanediylidene)dipyrrolo[3,2-i:3′,4′- l][1,4,7]oxadiazacyclopentadecine- 4,16(1H,15H)-dione |
| 145 | 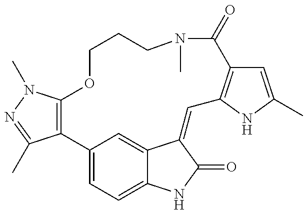 [SMILES: Cc1cc2c([nH]1)/C=C1\C(=O)Nc3ccc(cc31)-c1c(C)nn(C)c1OCCCN(C)C2=O] | [3a(4)Z]-6,9,14,16-tetramethyl- 9,10,11,12-tetrahydro-14H-1,17- (ethanediylidene)pyrazolo[4,3- n]dipyrrolo[3,2-g:3′,4′- j][1,5]oxazacyclopentadecine- 3,8(2H,5H)-dione |
| 146 | 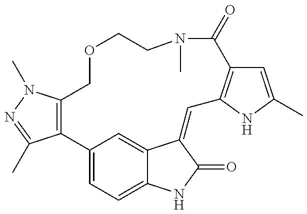 [SMILES: Cc1cc2c([nH]1)/C=C1\C(=O)Nc3ccc(cc31)-c1c(C)nn(C)c1COCCN(C)C2=O] | [3a(4)Z]-6,9,14,16-tetramethyl- 10,11,13,14-tetrahydro-2H-1,17- (ethanediylidene)pyrazolo[4,3- m]dipyrrolo[3,2-f:3′,4′- i][1,4]oxazacyclopentadecine- 3,8(5H,9H)-dione |
| 147 | 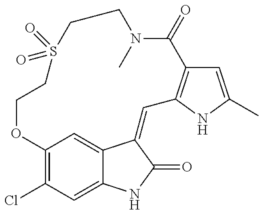 [SMILES: Cc1cc2c([nH]1)/C=C1\C(=O)Nc3cc(Cl)c(cc31)OCCS(=O)(=O)CCN(C)C2=O] | [16a(17)Z]-19-chloro-2,5-dimethyl- 6,7,9,10-tetrahydro-12,14- (ethanediylidene)-8λ⁶-dipyrrolo[3,2- i:3′,4′- l][1,4,7]oxathiazacyclopentadecine- 4,8,8,16(1H,5H,15H)-tetrone |
| 148 | 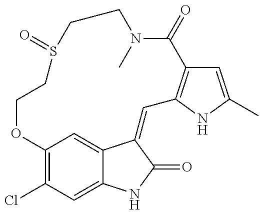 [SMILES: Cc1cc2c([nH]1)/C=C1\C(=O)Nc3cc(Cl)c(cc31)OCCS(=O)CCN(C)C2=O] | [16a(17)Z]-19-chloro-2,5-dimethyl- 6,7,9,10-tetrahydro-12,14- (ethanediylidene)-8λ⁴-dipyrrolo[3,2- i:3′,4′- l][1,4,7]oxathiazacyclopentadecine- 4,8,16(1H,5H,15H)-trione |
| 149 | 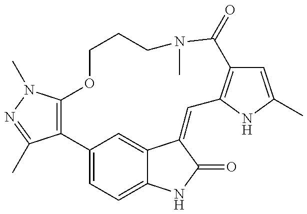 [SMILES: Cc1cc2c([nH]1)/C=C1\C(=O)Nc3ccc(cc31)-c1c(C)nn(C)c1OCCCN(C)C2=O] | [3a(4)Z]-9,14,16-trimethyl- 9,10,11,12-tetrahydro-14H-1,17- (ethanediylidene)pyrazolo[4,3- n]dipyrrolo[3,2-g:3′,4′- j][1,5]oxazacyclopentadecine- 3,8(2H,5H)-dione |
| 150 | 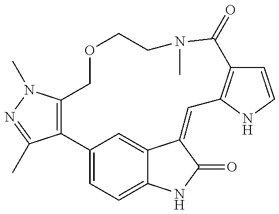 [SMILES: Cc1nn(C)c2c1-c1ccc3c(c1)/C(=C/c1[nH]ccc1C(=O)N(C)CCOC2)C(=O)N3] | [3a(4)Z]-9,14,16-trimethyl- 10,11,13,14-tetrahydro-2H-1,17- (ethanediylidene)pyrazolo[4,3- m]dipyrrolo[3,2-f:3′,4′- i][1,4]oxazacyclopentadecine- 3,8(5H,9H)-dione |
| 151 | 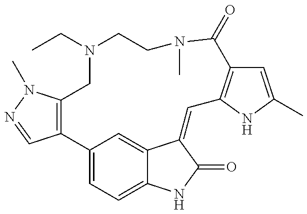 [SMILES: CCN1CCN(C)C(=O)c2cc(C)[nH]c2/C=C2\C(=O)Nc3ccc(cc32)-c2cnn(C)c2C1] | [3a(4)Z]-12-ethyl-6,9,14-trimethyl- 9,10,11,12,13,14-hexahydro-1,17- (ethanediylidene)pyrazolo[4,3- m]dipyrrolo[3,2-f:3′,4′- i][1,4]diazacyclopentadecine- 3,8(2H,5H)-dione |
| 152 | 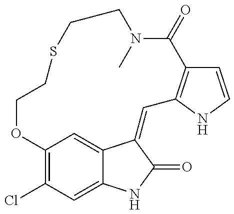 [SMILES: CN1CCSCCOc2cc3c(cc2Cl)NC(=O)/C3=C\c2[nH]ccc2C1=O] | [16a(17)Z]-19-chloro-5-methyl- 6,7,9,10-tetrahydro-1H-12,14- (ethanediylidene)dipyrrolo[3,2-i:3′,4′- l][1,4,7]oxathiazacyclopentadecine- 4,16(5H,15H)-dione |
| 153 | 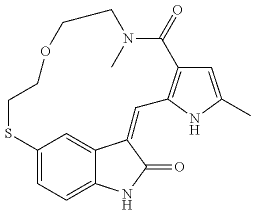 [SMILES: Cc1cc2c([nH]1)/C=C1\C(=O)Nc3ccc(cc31)SCCOCCN(C)C2=O] | [16a(17)Z]-2,5-dimethyl-6,7,9,10- tetrahydro-1H-12,14- (ethanediylidene)dipyrrolo[3,4- g:2′,3′- j][1,4,13]oxathiazacyclopentadecine- 4,16(5H,15H)-dione |
| 154 | 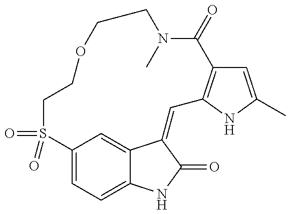 [SMILES: Cc1cc2c([nH]1)/C=C1\C(=O)Nc3ccc(cc31)S(=O)(=O)CCOCCN(C)C2=O] | [16a(17)Z]-2,5-dimethyl-6,7-dihydro- 1H,9H-12,14-(ethanediylidene)-11λ⁶- dipyrrolo[3,4-g:2′,3′- j][1,4,13]oxathiazacyclopentadecine- 4,11,11,16(5H,10H,15H)-tetrone |
| 155 | 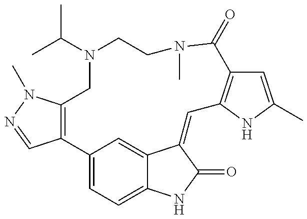 [SMILES: Cc1cc2c([nH]1)/C=C1\C(=O)Nc3ccc(cc31)-c1cnn(C)c1CN(C(C)C)CCN(C)C2=O] | [3a(4)Z]-6,9,14-trimethyl-12-(propan- 2-yl)-9,10,11,12,13,14-hexahydro- 1,17-(ethanediylidene)pyrazolo[4,3- m]dipyrrolo[3,2-f:3′,4′- i][1,4]diazacyclopentadecine- 3,8(2H,5H)-dione |
| 156 | 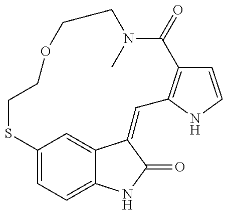 [SMILES: CN1CCOCCSc2ccc3c(c2)/C(=C/c2[nH]ccc2C1=O)C(=O)N3] | [16a(17)Z]-5-methyl-6,7,9,10- tetrahydro-1H-12,14- (ethanediylidene)dipyrrolo[3,4- g:2′,3′- j][1,4,13]oxathiazacyclopentadecine- 4,16(5H,15H)-dioen |
| 157 | 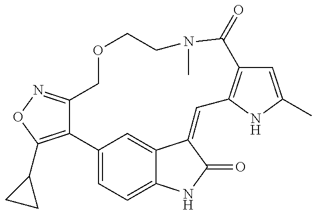 [SMILES: Cc1cc2c([nH]1)/C=C1\C(=O)Nc3ccc(cc31)-c1c(noc1C1CC1)COCCN(C)C2=O] | [3a(4)Z]-16-cylopropyl-6,9- dimethyl-10,11-dihydro-2H,13H- 1,17- (ethanediylidene)[1,2]oxazolo[4,3- m]dipyrrolo[3,2-f:3′,4′- i][1,4]oxazacyclopentadecine- 3,8(5H,9H)-dione |
| 158 | 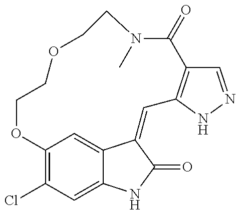 [SMILES: CN1CCOCCOc2cc3c(cc2Cl)NC(=O)/C3=C\c2[nH]ncc2C1=O] | [16a(17)Z]-19-chloro-5-methyl- 6,7,9,10-tetrahydro-1H-12,14- (ethanediylidene)pyrazolo[4,3- i]pyrrolo[3,4- l][1,4,7]dioxazacyclopentadecine- 4,16(5H,15H)-dione |
| 159 | 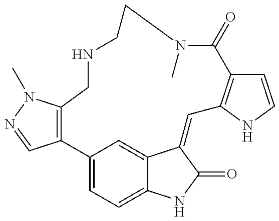 [SMILES: CN1CCNCc2c(cnn2C)-c2ccc3c(c2)/C(=C/c2[nH]ccc2C1=O)C(=O)N3] | [3a(4)Z]-9,14-dimethyl- 9,10,11,12,13,14-hexahydro-1,17- (ethanediylidene)pyrazolo[4,3- m]dipyrrolo[3,2-f:3′,4′- i][1,4]diazacyclopentadecine- 3,8(2H,5H)-dione |
| 160 | 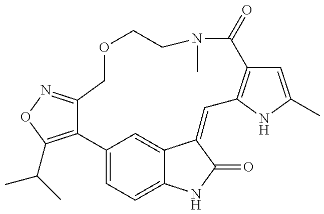 [SMILES: Cc1cc2c([nH]1)/C=C1\C(=O)Nc3ccc(cc31)-c1c(noc1C(C)C)COCCN(C)C2=O] | [3a(4)Z]-6,9-dimethyl-16-(propan-2- yl)-10,11-dihydro-2H,13H-1,17- (ethanediylidene)[1,2]oxazolo[4,3- m]dipyrrolo[3,2-f:3′,4′- i][1,4]oxazacyclopentadecine 3,8(5H,9H)-dione |
| 161 | 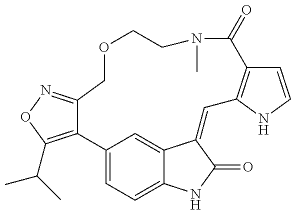 [SMILES: CC(C)c1onc2c1-c1ccc3c(c1)/C(=C/c1[nH]ccc1C(=O)N(C)CCOC2)C(=O)N3] | [3a(4)Z]-9-methyl-16-(propan-2-yl)- 10,11-dihydro-2H,13H-1,17- (ethanediylidene)[1,2]oxazolo[4,3- m]dipyrrolo[3,2-f:3′,4′- i][1,4]oxazacyclopentadecine- 3,8(5H,9H)-dione |
| 162 | 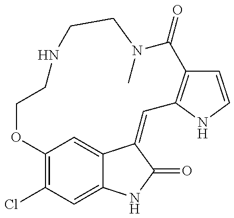 [SMILES: CN1CCNCCOc2cc3c(cc2Cl)NC(=O)/C3=C\c2[nH]ccc2C1=O] | [16a(17)Z]-19-chloro-5-methyl- 5,6,7,8,9,10-hexahydro-12,14- (ethanediylidene)dipyrrolo[3,2-i:3′,4′- l][1,4,7]oxadiazacyclopentadecine- 4,16(1H,15H)-dione |
| 163 | 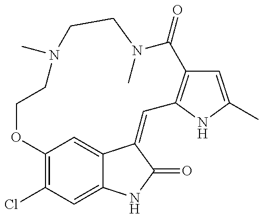 [SMILES: Cc1cc2c([nH]1)/C=C1\C(=O)Nc3cc(Cl)c(cc31)OCCN(C)CCN(C)C2=O] | [16a(17)Z]-19-chloro-2,5,8-trimethyl- 5,6,7,8,9,10-hexahydro-12,14- (ethanediylidene)dipyrrolo[3,2-i:3′,4′- l][1,4,7]oxadiazacyclopentadecine- 4,16(1H,15H)-dione |
| 164 | 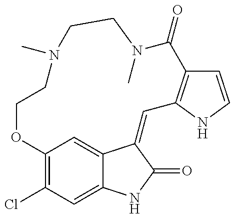 [SMILES: CN1CCOc2cc3c(cc2Cl)NC(=O)/C3=C\c2[nH]ccc2C(=O)N(C)CC1] | [16a(17)Z]-19-chloro-5,8-dimethyl- 5,6,7,8,9,10-hexahydro-12,14- (ethanediylidene)dipyrrolo[3,2-i:3′,4′- l][1,4,7]oxadiazacyclopentadecine- 4,16(1H,15H)-dione |
| 165 | 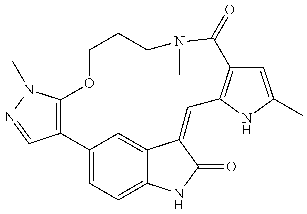 [SMILES: Cc1cc2c([nH]1)/C=C1\C(=O)Nc3ccc(cc31)-c1cnn(C)c1OCCCN(C)C2=O] | [3a(4)Z]-6,9,14-trimethyl-9,10,11,12- tetrahydro-14H-1,17- (ethanediylidene)pyrazolo[4,3- n]dipyrrolo[3,2-g:3′,4′- j][1,5]oxazacyclopentadecine- 3,8(2H,5H)-dione |
| 166 | 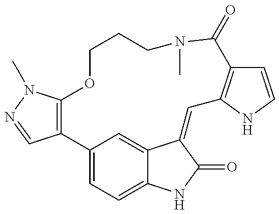 [SMILES: CN1CCCOc2c(cnn2C)-c2ccc3c(c2)/C(=C/c2[nH]ccc2C1=O)C(=O)N3] | [3a(4)Z]-9,14-dimethyl-9,10,11,12- tetrahydro-14H-1,17- (ethanediylidene)pyrazolo[4,3- n]dipyrrolo[3,2-g:3′,4′- j][1,5]oxazacyclopentadecine- 3,8(2H,5H)-dione |
| 167 | 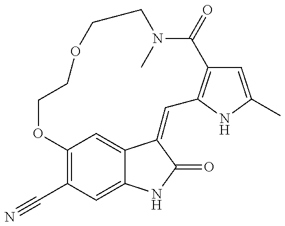 [SMILES: Cc1cc2c([nH]1)/C=C1\C(=O)Nc3cc(C#N)c(cc31)OCCOCCN(C)C2=O] | [16a(17)Z]-2,5-dimethyl-4,16-dioxo- 4,5,6,7,9,10,15,16-octahydro-1H- 12,14-(ethanediylidene)dipyrrolo[3,2- i:3′,4′- l][1,4,7]dioxazacyclopentadecine-19- carbonitrile |
| 168 | 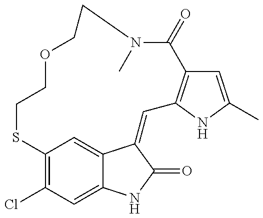 [SMILES: Cc1cc2c([nH]1)/C=C1\C(=O)Nc3cc(Cl)c(cc31)SCCOCCN(C)C2=O] | [16a(17)Z]-19-chloro-2,5-dimethyl- 6,7,9,10-tetrahydro-1H-12,14- (ethanediylidene)dipyrrolo[3,4- g:2′,3′- j][1,4,13]oxathiazacyclopentadecine- 4,16(5H,15H)-dione |
| 169 | 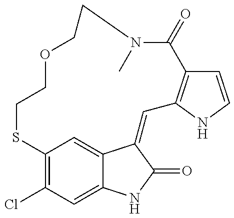 [SMILES: CN1CCOCCSc2cc3c(cc2Cl)NC(=O)/C3=C\c2[nH]ccc2C1=O] | [16a(17)Z]-19-chloro-5-methyl- 6,7,9,10-tetrahydro-1H-12,14- (ethanediylidene)dipyrrolo[3,4- g:2′,3′- j][1,4,13]oxathiazacyclopentadecine- 4,16(5H,15H)-dione |
| 170 | 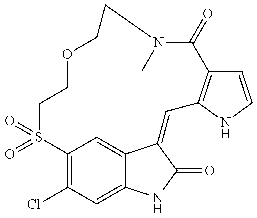 [SMILES: CN1CCOCCS(=O)(=O)c2cc3c(cc2Cl)NC(=O)/C3=C\c2[nH]ccc2C1=O] | [16a(17)Z]-19-chloro-5-methyl-6,7- dihydro-1H,9H-12,14- (ethanediylidene)-11λ⁶-dipyrrolo[3,4- g:2′,3′- j][1,4,13]oxathiazacyclopentadecine- 4,11,11,16(5H,10H,15H)-tetrone |
| 171 | 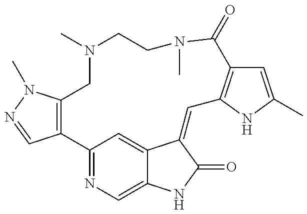 [SMILES: Cc1cc2c([nH]1)/C=C1\C(=O)Nc3cnc(cc31)-c1cnn(C)c1CN(C)CCN(C)C2=O] | [3a(4)Z]-6,9,12,14-tetramethyl- 9,10,11,12,13,14-hexahydro-17,1- (azenometheno)pyrazolo[4,3- m]dipyrrolo[3,2-f:3′,4′- i][1,4]diazacyclopentadecine- 3,8(2H,5H)-dione |
| 172 | 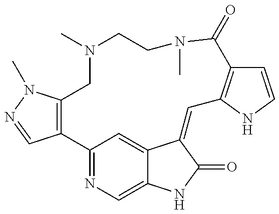 [SMILES: CN1CCN(C)C(=O)c2cc[nH]c2/C=C2\C(=O)Nc3cnc(cc32)-c2cnn(C)c2C1] | [3a(4)Z]-9,12,14-trimethyl- 9,10,11,12,13,14-hexahydro-17,1- (azenometheno)pyrazolo[4,3- m]dipyrrolo[3,2-f:3′,4′- i][1,4]diazacyclopentadecine- 3,8(2H,5H)-dione |
| 173 | 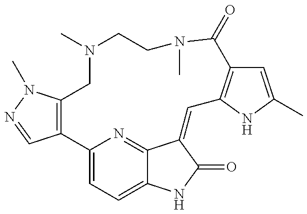 [SMILES: Cc1cc2c([nH]1)/C=C1\C(=O)Nc3ccc(nc31)-c1cnn(C)c1CN(C)CCN(C)C2=O] | [3a(4)Z]-6,9,12,14-tetramethyl- 9,10,11,12,13,14-hexahydro-1,17- (ethanediylidene)pyrazolo[3,4- f]dipyrrolo[3,4-j:2′,3′- m][1,4,]triazacyclopentadecine- 3,8(2H,5H)-dione |
| 174 | 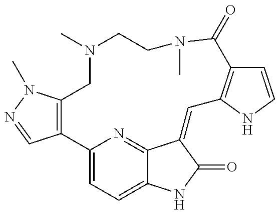 [SMILES: CN1CCN(C)C(=O)c2cc[nH]c2/C=C2\C(=O)Nc3ccc(nc32)-c2cnn(C)c2C1] | [3a(4)Z]-9,12,14-trimethyl- 9,10,11,12,13,14-hexahydro-1,17- (ethanediylidene)pyrazolo[3,4- f]dipyrrolo[3,4-j:2′,3′- m][1,4,9]triazacyclopentadecine- 3,8(2H,5H)-dione |

and pharmaceutically acceptable salts thereof.

Those skilled in the art will recognize that the species listed or illustrated herein are not exhaustive, and that additional species within the scope of these defined terms may also be selected.

### PHARMACEUTICAL COMPOSITIONS

For treatment purposes, pharmaceutical compositions comprising the compounds described herein may further comprise one or more pharmaceutically-acceptable excipients. A pharmaceutically-acceptable excipient is a substance that is non-toxic and otherwise biologically suitable for administration to a subject. Such excipients facilitate administration of the compounds described herein and are compatible with the active ingredient. Examples of pharmaceutically-acceptable excipients include stabilizers, lubricants, surfactants, diluents, anti-oxidants, binders, coloring agents, bulking agents, emulsifiers, or taste-modifying agents. In preferred embodiments, pharmaceutical compositions according to the disclosure are sterile compositions. Pharmaceutical compositions may be prepared using compounding techniques known or that become available to those skilled in the art.

Sterile compositions are also contemplated by the disclosure, including compositions that are in accord with national and local regulations governing such compositions.

The pharmaceutical compositions and compounds described herein may be formulated as solutions, emulsions, suspensions, or dispersions in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. Pharmaceutical compositions of the disclosure may be administered by a suitable route of delivery, such as oral, parenteral, rectal, nasal, topical, or ocular routes, or by inhalation. Preferably, the compositions are formulated for intravenous or oral administration.

For oral administration, the compounds the disclosure may be provided in a solid form, such as a tablet or capsule, or as a solution, emulsion, or suspension. To prepare the oral compositions, the compounds of the disclosure may be formulated to yield a dosage of, e.g., from about 0.1 mg to 1 g daily, or about 1 mg to 50 mg daily, or about 50 to 250 mg daily, or about 250 mg to 1 g daily. Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid, or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil, such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions, or syrups, or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

For parenteral use, including intravenous, intramuscular, intraperitoneal, intranasal, or subcutaneous routes, the agents of the disclosure may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampoules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent admixed with a pharmaceutical carrier over a period ranging from several minutes to several days.

For nasal, inhaled, or oral administration, the inventive pharmaceutical compositions may be administered using, for example, a spray formulation also containing a suitable carrier. The inventive compositions may be formulated for rectal administration as a suppository.

For topical applications, the compounds of the present disclosure are preferably formulated as creams or ointments or a similar vehicle suitable for topical administration. For topical administration, the inventive compounds may be mixed with a pharmaceutical carrier at a concentration of about 0.1% to about 10% of drug to vehicle. Another mode of administering the agents of the disclosure may utilize a patch formulation to effect transdermal delivery.

As used herein, the terms “treat” or “treatment” encompass both “preventative” and “curative” treatment. “Preventative” treatment is meant to indicate a postponement of development of a disease, a symptom of a disease, or medical condition, suppressing symptoms that may appear, or reducing the risk of developing or recurrence of a disease or symptom. “Curative” treatment includes reducing the severity of or suppressing the worsening of an existing disease, symptom, or condition. Thus, treatment includes ameliorating or preventing the worsening of existing disease symptoms, preventing additional symptoms from occurring, ameliorating or preventing the underlying systemic causes of symptoms, inhibiting the disorder or disease, e.g., arresting the development of the disorder or disease, relieving the disorder or disease, causing regression of the disorder or disease, relieving a condition caused by the disease or disorder, or stopping the symptoms of the disease or disorder.

The term “subject” refers to a mammalian patient in need of such treatment, such as a human.

Exemplary diseases include cancer, pain, neurological diseases, autoimmune diseases, and inflammation. As used herein, the term “cancer” includes, but is not limited to, ALCL, NSCLC, neuroblastoma, inflammatory myofibroblastic tumor, adult renal cell carcinoma, pediatric renal cell carcinoma, breast cancer, ER⁺ breast cancer, colonic adenocarcinoma, glioblastoma, glioblastoma multiforme, anaplastic thyroid cancer, cholangiocarcinoma, ovarian cancer, gastric adenocarcinoma, colorectal cancer, inflammatory myofibroblastic tumor, angiosarcoma, epithelioid hemangioendothelioma, intrahepatic cholangiocarcinoma, thyroid papillary cancer, spitzoid neoplasms, sarcoma, astrocytoma, brain lower grade glioma, secretory breast carcinoma, mammary analogue carcinoma, acute myeloid leukemia, congenital mesoblastic nephroma, congenital fibrosarcomas, Ph-like acute lymphoblastic leukemia, thyroid carcinoma, skin cutaneous melanoma, head and neck squamous cell carcinoma, pediatric glioma CML, prostate cancer, lung squamous carcinoma, ovarian serous cystadenocarcinoma, skin cutaneous melanoma, castrate-resistant prostate cancer, Hodgkin lymphoma, and serous and clear cell endometrial cancer. In some embodiments, cancer includes, lung cancer, colon cancer, breast cancer, prostate cancer, hepatocellular carcinoma, renal cell carcinoma, gastric and esophago-gastric cancers, glioblastoma, head and neck cancers, inflammatory myofibroblastic tumors, and anaplastic large cell lymphoma. Pain includes, for example, pain from any source or etiology, including cancer pain, pain from chemotherapeutic treatment, nerve pain, pain from injury, or other sources. Autoimmune diseases include, for example, rheumatoid arthritis, Sjogren syndrome, Type I diabetes, and lupus. Exemplary neurological diseases include Alzheimer's Disease, Parkinson's Disease, Amyotrophic lateral sclerosis, and Huntington's disease. Exemplary inflammatory diseases include atherosclerosis, allergy, and inflammation from infection or injury.

In one aspect, the compounds and pharmaceutical compositions of the disclosure specifically target tyrosine receptor kinases, in particular EGFR. Thus, these compounds and pharmaceutical compositions can be used to prevent, reverse, slow, or inhibit the activity of one or more of these kinases. In preferred embodiments, methods of treatment target cancer. In other embodiments, methods are for treating lung cancer or non-small cell lung cancer.

In the inhibitory methods of the disclosure, an “effective amount” means an amount sufficient to inhibit the target protein. Measuring such target modulation may be performed by routine analytical methods such as those described below. Such modulation is useful in a variety of settings, including in vitro assays. In such methods, the cell is preferably a cancer cell with abnormal signaling due to upregulation of EGFR.

In treatment methods according to the disclosure, an “effective amount” means an amount or dose sufficient to generally bring about the desired therapeutic benefit in subjects needing such treatment. Effective amounts or doses of the compounds of the disclosure may be ascertained by routine methods, such as modeling, dose escalation, or clinical trials, taking into account routine factors, e.g., the mode or route of administration or drug delivery, the pharmacokinetics of the agent, the severity and course of the infection, the subject's health status, condition, and weight, and the judgment of the treating physician. An exemplary dose is in the range of about from about 0.1 mg to 1 g daily, or about 1 mg to 50 mg daily, or about 50 to 250 mg daily, or about 250 mg to 1 g daily. The total dosage may be given in single or divided dosage units (e.g., BID, TID, QID).

Once improvement of the patient's disease has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms. Patients may also require chronic treatment on a long-term basis.

### Drug Combinations

The inventive compounds described herein may be used in pharmaceutical compositions or methods in combination with one or more additional active ingredients in the treatment of the diseases and disorders described herein. Further additional active ingredients include other therapeutics or agents that mitigate adverse effects of therapies for the intended disease targets. Such combinations may serve to increase efficacy, ameliorate other disease symptoms, decrease one or more side effects, or decrease the required dose of an inventive compound. The additional active ingredients may be administered in a separate pharmaceutical composition from a compound of the present disclosure or may be included with a compound of the present disclosure in a single pharmaceutical composition. The additional active ingredients may be administered simultaneously with, prior to, or after administration of a compound of the present disclosure.

Combination agents include additional active ingredients are those that are known or discovered to be effective in treating the diseases and disorders described herein, including those active against another target associated with the disease. For example, compositions and formulations of the disclosure, as well as methods of treatment, can further comprise other drugs or pharmaceuticals, e.g., other active agents useful for treating or palliative for the target diseases or related symptoms or conditions. For cancer indications, additional such agents include, but are not limited to, kinase inhibitors, such as ALK inhibitors (e.g. crizotinib), Raf inhibitors (e.g., vemurafenib), VEGFR inhibitors (e.g., sunitinib), standard chemotherapy agents such as alkylating agents, antimetabolites, anti-tumor antibiotics, topoisomerase inhibitors, platinum drugs, mitotic inhibitors, antibodies, hormone therapies, or corticosteroids. For pain indications, suitable combination agents include anti-inflammatories such as NSAIDs. The pharmaceutical compositions of the disclosure may additional comprise one or more of such active agents, and methods of treatment may additionally comprise administering an effective amount of one or more of such active agents.

### Chemical Synthesis Methods

The following examples are offered to illustrate but not to limit the disclosure. One of skill in the art will recognize that the following synthetic reactions and schemes may be modified by choice of suitable starting materials and reagents in order to access other compounds of Formula (I)-(VIII).

In some embodiments, the disclosure provides compounds of the formula (IX)

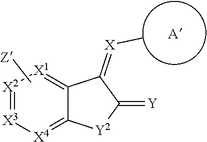

A′ is a 5- to 10-membered heteroaryl or C₆-C₁₀ aryl, optionally substituted with one or more of deuterium, halogen, —OC₁-C₆ alkyl, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —C(S)R^{a}, —C(S)OR^{a}, —C(S)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in —OC₁-C₆ alkyl, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
Z′ is a 3- to 7-membered heterocycloalkyl, C₃-C₆ cycloalkyl, C₆-C₁₀ aryl, 5- to 10-membered heteroaryl, —C(R^{a})(R^{b})H, —C(O)R^{a}, —OR^{a}, —NR^{a}R^{b}, —SR^{a}, —S(O)R^{a} or —S(O)₂R^{a}, wherein each hydrogen atom in 3- to 7-membered heterocycloalkyl, C₃-C₆ cycloalkyl, C₆-C₁₀ aryl, and 5- to 10-membered heteroaryl is independently optionally substituted by deuterium, halogen, C₁-C₆ alkyl, C₁-C₆ haloalkyl, C₂-C₆ alkenyl, C₂-C₆ alkynyl, C₃-C₆ cycloalkyl, 3- to 7-membered heterocycloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
X is —N— or —C(R⁶)—;
X¹ is —N—, —C(R⁷)—, or a bond to Z′; X² is —N—, —C(R⁸)—, or a bond to Z′; provided that one of X¹ or X² is a bond to Z′;
X³ is —N— or —C(R⁹)—;
X⁴ is —N— or —C(R¹¹)—;
Y is —O— or —S—;
Y² is —O—, —N(R¹¹)—, or —S—;
R⁶ is H, deuterium, halogen, C₁-C₆ alkyl, C₂-C₆ alkenyl, C₂-C₆ alkynyl, or —CN;
each of R⁷, R⁸, R⁹, and R¹⁰ is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OC₁-C₆ alkyl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂; or R³ and R⁴ or R⁴ and R⁵ taken together with the carbons to which they are attached form a C₄-C₆ cycloalkyl, a 4- to 7-membered heterocycloalkyl, or a C₆-C₁₀ aryl, wherein each hydrogen atom in C₁-C₆ alkyl, C₂-C₆ alkenyl, C₂-C₆ alkynyl, C₃-C₆ cycloalkyl, C₄-C₆ cycloalkyl, 3- to 7-membered heterocycloalkyl, C₆-C₁₀ aryl, 5- to 10-membered heteroaryl, or 4- to 7-membered heterocycloalkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R¹¹ is independently H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}′, —CN, or —NO₂;
each R^{a}, R^{b}, R^{e}, R^{d}, R^{e}, and R^{f} is independently selected from the group consisting of H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkyl-C₆-C₁₀ aryl, and 5- to 10-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkyl-C₆-C₁₀ aryl, and 5- to 10-membered heteroaryl is independently optinally substituted by —OH, —OPG, —CN, —OC₁-C₆ alkyl, —NH₂, —NHPG, —NH(C₁-C₆ alkyl), —N(C₁-C₆ alkyl)₂, —NHC(O)C₁-C₆ alkyl, —N(C₁-C₆ alkyl)C(O)C₁-C₆ alkyl, —NHC(O)NH₂, —NHC(O)NHC₁-C₆ alkyl, —N(C₁-C₆ alkyl)C(O)NH₂, —N(C₁-C₆ alkyl)C(O)NHC₁-C₆ alkyl, —NHC(O)N(C₁-C₆ alkyl)₂, —N(C₁-C₆ alkyl)C(O)N(C₁-C₆ alkyl)₂, —NHC(O)OC₁-C₆ alkyl, —N(C₁-C₆ alkyl)C(O)OC₁-C₆ alkyl, —NHS(O)(C₁-C₆ alkyl), —NHS(O)₂(C₁-C₆ alkyl), —N(C₁-C₆ alkyl)S(O)(C₁-C₆ alkyl), —N(C₁-C₆ alkyl)S(O)₂(C₁-C₆ alkyl), —NHS(O)NH₂, —NHS(O)₂NH₂, —N(C₁-C₆ alkyl)S(O)NH₂, —N(C₁-C₆ alkyl)S(O)₂NH₂, —NHS(O)NH(C₁-C₆ alkyl), —NHS(O)₂NH(C₁-C₆ alkyl), —NHS(O)N(C₁-C₆ alkyl)₂, —NHS(O)₂N(C₁-C₆ alkyl)₂, —N(C₁-C₆ alkyl)S(O)NH(C₁-C₆ alkyl), —N(C₁-C₆ alkyl)S(O)₂NH(C₁-C₆ alkyl), —N(C₁-C₆ alkyl)S(O)N(C₁-C₆ alkyl)₂, —N(C₁-C₆ alkyl)S(O)₂N(C₁-C₆ alkyl)₂, —CO₂H, —COOPG, —C(O)OC₁-C₆ alkyl, —C(O)NH₂, —C(O)NHPG, —C(O)NH(C₁-C₆ alkyl), —C(O)N(C₁-C₆ alkyl)₂, —SC₁—C₆ alkyl, —S(O)C₁-C₆ alkyl, —S(O)₂C₁-C₆ alkyl, —S(O)NH(C₁-C₆ alkyl), —S(O)₂NH(C₁-C₆ alkyl), —S(O)N(C₁-C₆ alkyl)₂, —S(O)₂N(C₁-C₆ alkyl)₂, —P(C₁-C₆ alkyl)₂, —P(O)(C₁-C₆ alkyl)₂, C₃-C₆ cycloalkyl, or 3- to 7-membered heterocycloalkyl; and
PG is a protecting group.

Abbreviations: The examples described herein use materials, including but not limited to, those described by the following abbreviations known to those skilled in the art:

| | |
|---|---|
| g | grams |
| eq | equivalents |
| mmol | millimoles |
| mL | milliliters |
| EtOAc | ethyl acetate |
| MHz | megahertz |
| ppm | parts per million |
| δ | chemical shift |
| s | singlet |
| d | doublet |
| t | triplet |
| q | quartet |
| quin | quintet |
| br | broad |
| m | multiplet |
| Hz | hertz |
| THF | tetrahydrofuran |
| ° C. | degrees Celsius |
| PE | petroleum ether |
| EA | ethyl acetate |
| R_{f} | retardation factor |
| N | normal |
| J | coupling constant |
| DMSO-d₆ | deuterated dimethyl sulfoxide |
| n-BuOH | n-butanol |
| DIEA | n,n-diisopropylethylamine |
| TMSCl | trimethylsilyl chloride |
| min | minutes |
| hr | hours |
| Me | methyl |
| Et | ethyl |
| i-Pr | isopropyl |
| TLC | thin layer chromatography |
| M | molar |
| Compd# | compound number |
| MS | mass spectrum |
| m/z | mass-to-charge ratio |
| Ms | methanesulfonyl |
| FDPP | pentafluorophenyl diphenylphosphinate |
| Boc | tert-butyloxycarbonyl |
| TFA | trifluoroacetic acid |
| Tos | toluenesulfonyl |
| DMAP | 4-(dimethylamino)pyridine |
| mM | micromolar |
| ATP | adenosine triphosphate |
| IC₅₀ | half maximal inhibitory concentration |
| U/mL | units of activity per milliliter |
| KHMDS | potassium bis(trimethylsilyl)amide |
| DIAD | diisopropyl azodicarboxylate |
| MeTHF | 2-methyltetrahydrofuran |
| MOM | methoxymethyl |
| DCM | dichloromethane |
| DMF | N,N-dimethylformamide |
| DPPA | diphenyl phosphoryl azide |
| DBU | 1,8-diazabicyclo[5.4.0]undec-7-ene |
| DIPEA | N,N-diisopropylethylamine |
| SEM | [2-(Trimethylsilyl)ethoxy]methyl acetal |
| Hex | hexanes |
| Pd(dppf)Cl₂ | [1,1′-Bis(diphenylphosphino)ferrocene] |
| | dichloropalladium(II) |
| MeCN (ACN) | Acetonitrile |
| Pd₂(dba)₃ | Tris(dibenzylideneacetone)dipalladium(0) |
| Hunig's Base | N,N-diisopropylethylamine |
| TBAF | Tert butyl ammonium fluoride |
| PPh₃ | Triphenyl phosphine |
| RT | Room Temperature |
| p-TSA | Para-Tolylsulfonic acid |
| t-BuOH | Tert-Butanol |
| Pd(amphos)Cl₂ | Dichlorobis[di-tert-butyl(4- |
| | dimethylaminophenyl)phosphine]palladium(II) |
| mCPBA | Meta-Chloroperoxy benzoic acid |
| AcOH | Acetic Acid |
| DMAc | N, N-Dimethylformamide |
| BPD | 4,4,5,5-tetramethyl-2-(4,4,5,5-tetramethyl-1,3,2- |
| | dioxaborolan-2-yl)-1,3,2-dioxaborolane |
| MTBE | Methy tert-Butyl Ether |

General Method A

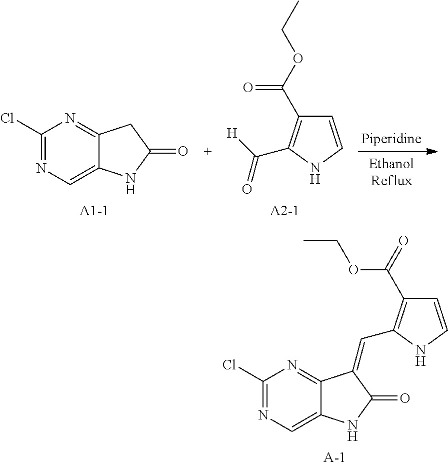

A mixture of the oxindole. A1-1 (1.0 equivalent (eq.)), the aldehyde A2-1 (1.0 eq.) and piperidine (2.0 eq.) in ethanol (0.4 M) is refluxed until reaction completion. The mixture is cooled to ambient temperature and the precipitated solid is collected by vacuum filtration, washed with ethanol and dried to give A-1. If a precipitate does not form upon cooling of the reaction mixture, the mixture is concentrated and purified by column chromatography.

The intermediates A-1-A-26 can be made via General Method A using the corresponding starting materials A1 and A2 as shown in the table below:

| A1 | A2 | A |
|---|---|---|
| 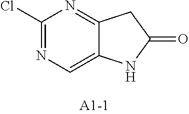 | 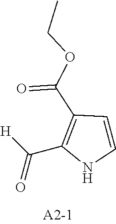 | 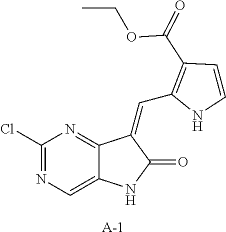 |
| 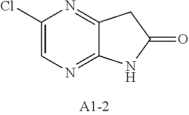 | 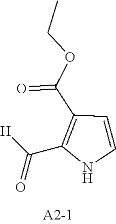 | 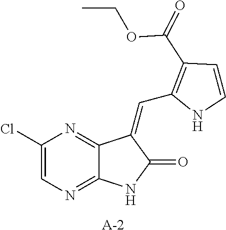 |
| 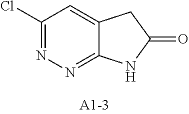 | 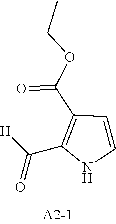 | 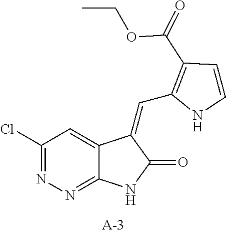 |
| 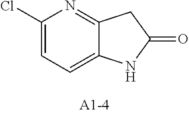 | 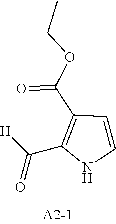 | 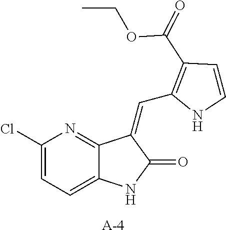 |
| 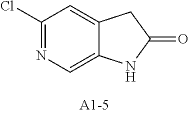 | 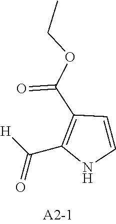 | 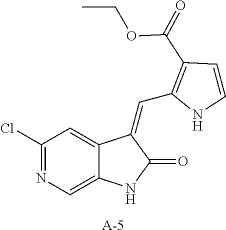 |
| 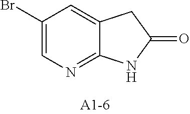 | 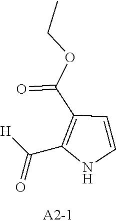 | 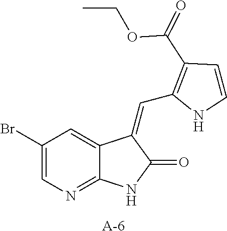 |
| 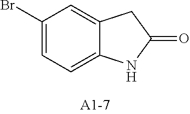 | 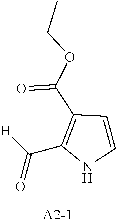 | 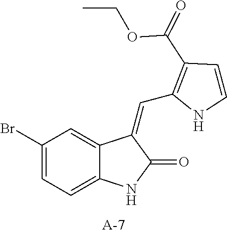 |
| 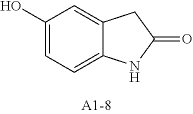 | 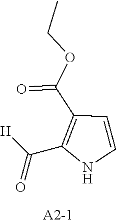 | 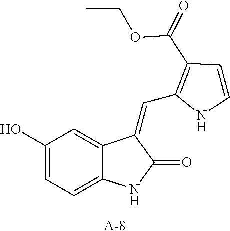 |
| 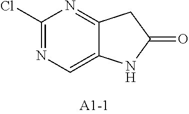 | 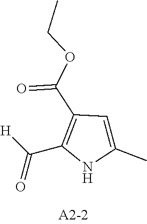 | 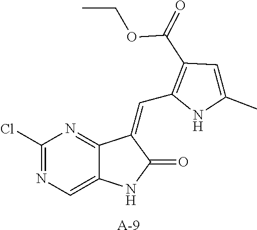 |
| 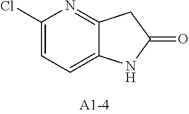 | 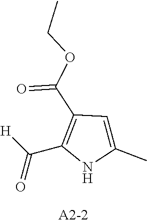 | 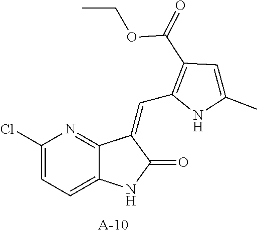 |
| 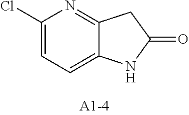 | 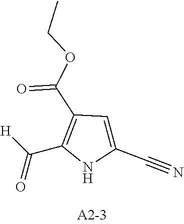 | 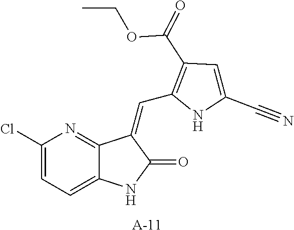 |
| 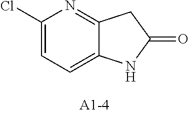 | 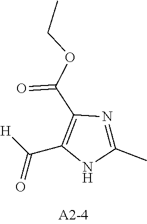 | 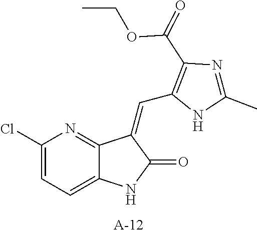 |
| 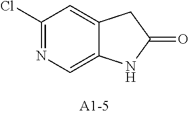 | 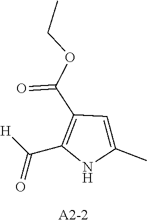 | 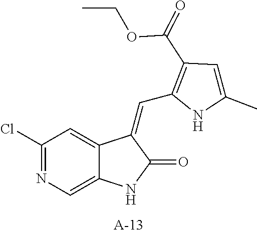 |
| 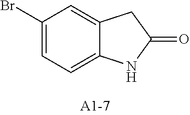 | 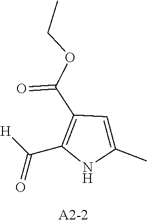 | 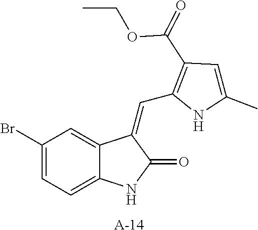 |
| 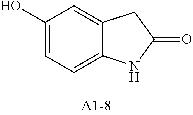 | 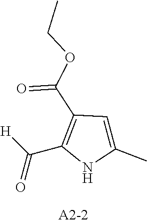 | 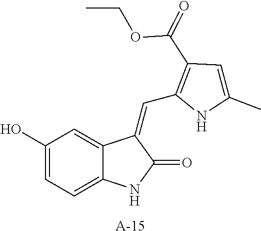 |
| 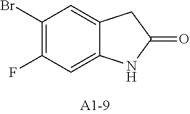 | 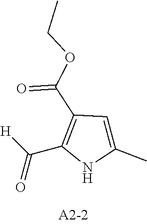 | 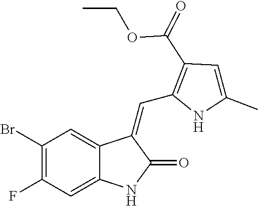 |
| 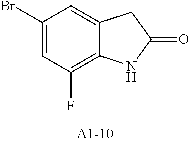 | 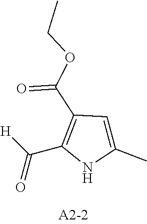 | 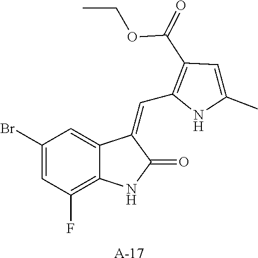 |
| 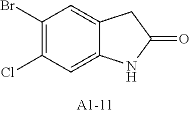 | 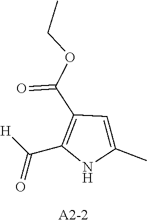 | 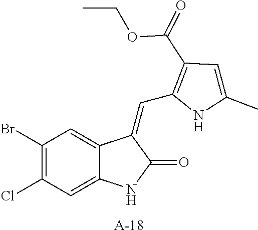 |
| 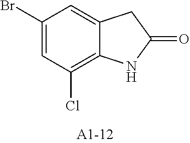 | 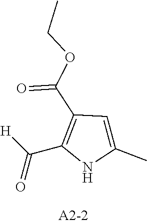 | 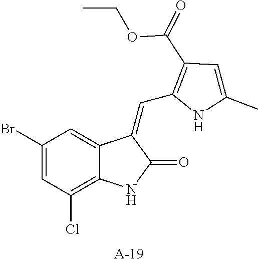 |
| 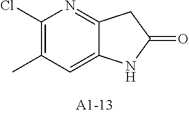 | 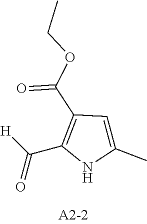 | 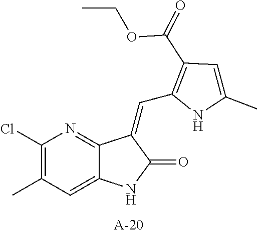 |
| 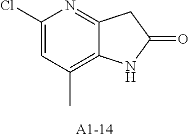 | 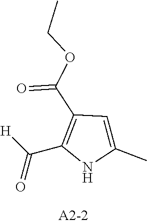 | 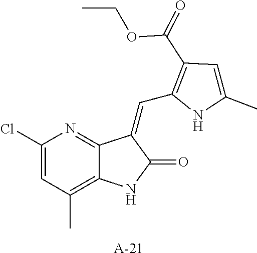 |
| 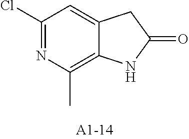 | 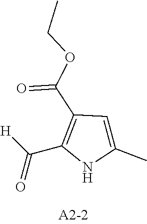 | 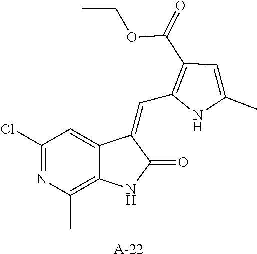 |
| 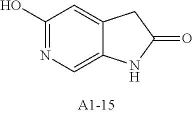 | 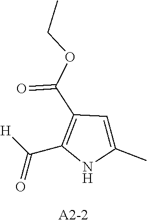 | 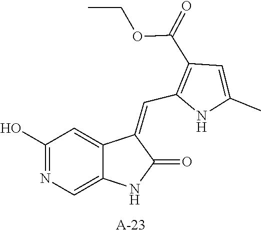 |
| 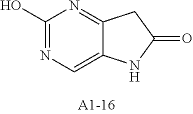 | 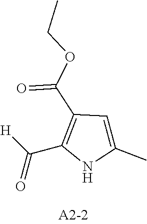 | 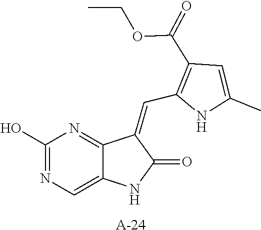 |
| 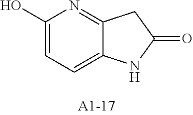 | 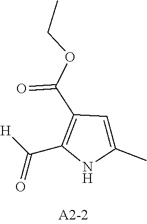 | 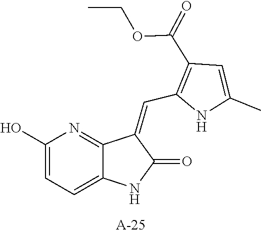 |
| 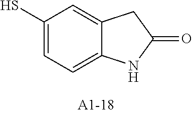 | 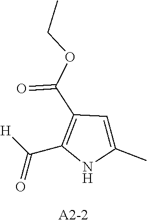 | 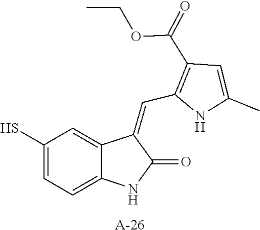 |

General Method B-I

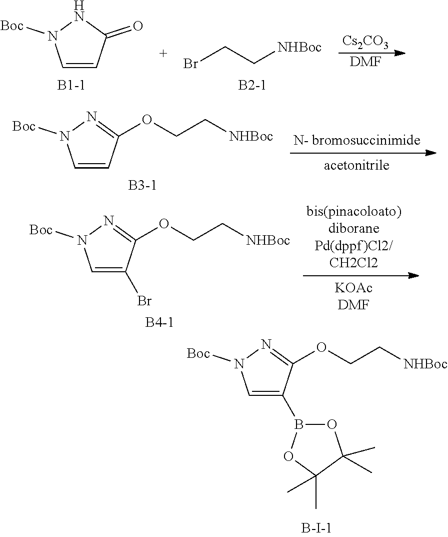

Step 1. To a solution of B1-1 (1.0 eq.) and B2-1 (1.5 eq.) in DMF (0.25 M) is added Cs₂CO₃ (2.0 eq.) and the mixture is heated at 60-80° C. under nitrogen until the reaction is completed. Water (5 equivalent volume of DMF) is added to the cooled DMF solution and the product was extracted with ethyl acetate (1 equivalent volume of water) for three times. The combined extracts are washed with water, aqueous HCl solution (1 N), brine, and dried over magnesium sulfate. After filtration and condensation, the crude product was purified on a silica gel column to provide pure product B3-1.

Alternative Step 1: BI-1 (1.0 eq.) is added to a suspension of NaH (60% in mineral oil, 1.1 eq.) in THF (0.5 M) at ambient temperature. After 30 min, to above suspension is added B2-1 (1.0 eq). After the reaction is complete, the reaction is quenched with saturated aqueous ammonium chloride solution and extracted with EtOAc for three times. The combined extracts are washed with brine, dried over Na₂SO₄, filtered, concentrated and purified on a silica gel column to provide B13-1.

Step 2. To a solution of B3-1 (1.0 eq.) in dry acetonitrile (0.25 M) is added N-bromosuccinimide (1.05 eq.) and the solution is stirred at ambient temperature until the reaction is completed. The reaction is quenched with aqueous sodium thiosulfate (0.1N) and the acetonitrile is then removed under vacuum. The residue is dissolved in water and extracted with ethyl acetate. The combined extracts are washed with water and brine, and then dried over magnesium sulfate. After filtration and condensation, the crude product was purified on a silica gel column to provide pure product B4-1.

Step 3. A mixture of B4-1 (1.0 eq), bis(pinacolato) diborane (1.2 eq), KOAc (3.0 eq), and catalyst Pd(dppf)Cl₂/CH₂Cl (0.05 eq) in anhydrous DMF (0.5 M) is purged with nitrogen gas. It is heated at about 95° C. under nitrogen for about 15 hours. The reaction solution is cooled down and diluted with ethyl acetate (5 volume of DMF) and filtered through a silica gel column, and concentrated. The residue is further purified by a silica gel flash chromatography to provide the pure product B-I-1.

The following pinacol boronates B-I-1-B-I-16 are prepared via the General Method B-I using the corresponding starting materials B1 and B2:

| B1 | B2 | B-I |
|---|---|---|
| 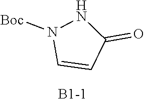 | 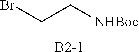 | 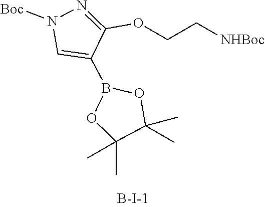 |
| 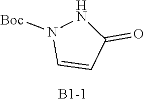 | 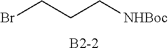 | 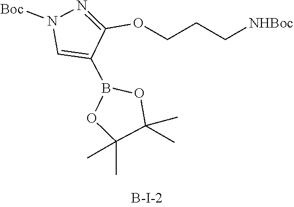 |
| 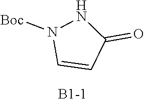 | 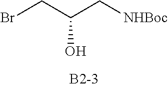 | 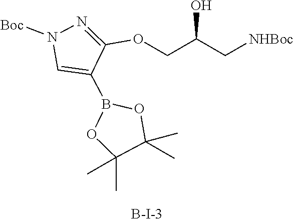 |
| 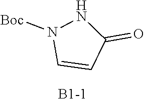 | 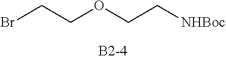 | 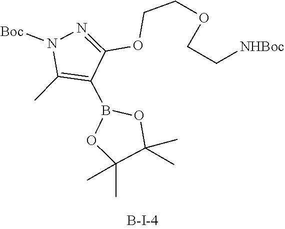 |
| 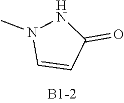 | 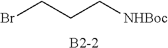 | 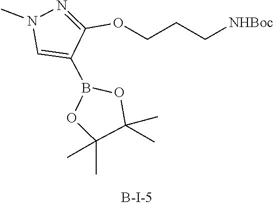 |
| 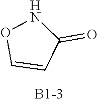 | 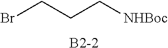 | 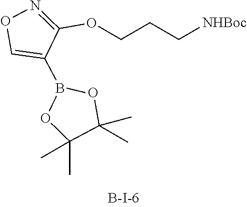 |
| 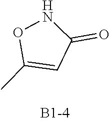 | 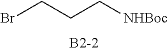 | 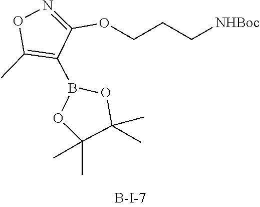 |
| 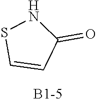 | 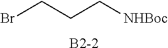 | 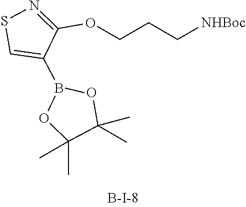 |
| 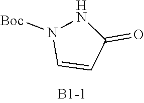 | 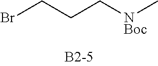 | 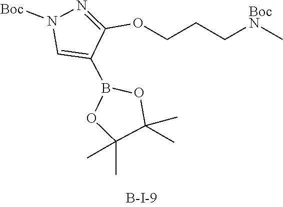 |
| 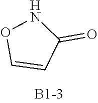 | 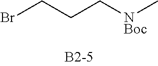 | 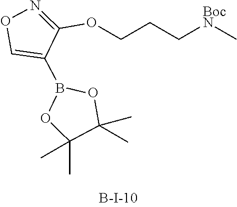 |
| 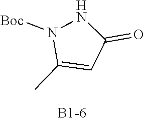 | 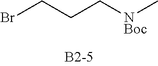 | 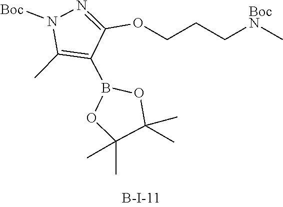 |
| 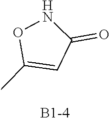 | 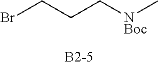 | 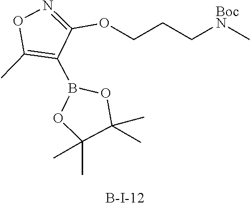 |
| 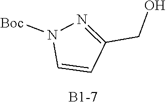 | 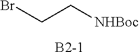 | 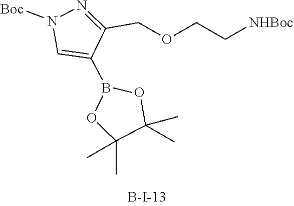 |
| 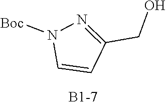 | 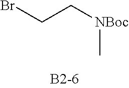 | 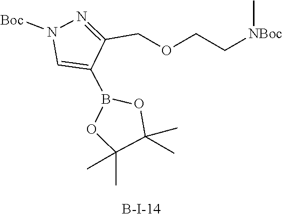 |
| 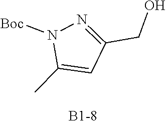 | 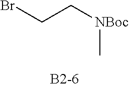 | 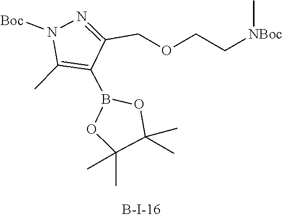 |

General Method B-II

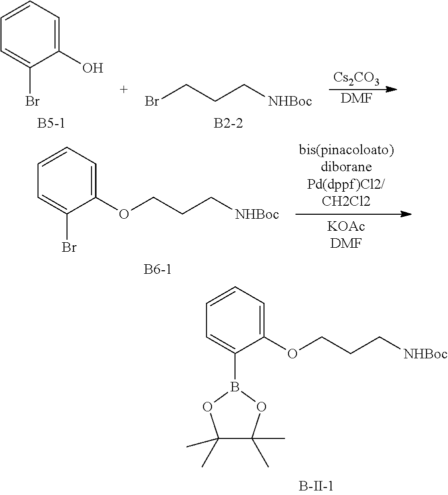

Step 1. To a solution of B5-1 (1.0 eq.) and B2-2 (1.5 eq.) in DMF (0.25 M) is added Cs₂CO₃ (2 eq.) and the mixture is heated at 60-80° C. under nitrogen until the reaction is completed. Water (5 volume of DMF) is added to the cooled DMF solution and the product was extracted with ethyl acetate (1 volume of water) for three times. The combined extracts are washed with water, aqueous HCl solution (1 N), brine, and dried over magnesium sulfate.

After filtration and condensation, the crude product was purified on a silica gel column to provide pure product B6-1.

Step 2. A mixture of B6-1 (1.0 eq), bis(pinacolato) diborane (1.2 eq), KOAc (3.0 eq), and catalyst Pd(dppf)Cl₂/CH₂Cl₂ (0.05 eq) in anhydrous DMF (0.5 M) is purged with nitrogen gas. It is heated at about 95° C. under nitrogen for about 15 hours. The reaction solution is cooled down and diluted with ethyl acetate (5 volume of DMF) and filtered through a silica gel column, and concentrated. The residue is further purified by a silica gel flash chromatography to provide the pure product B-II-1.

The following pinacol boronates B-II-1-1B-II-10 are prepared via the General Method B-II using the corresponding starting materials B5 and B2 as shown in the table below:

| B5 | B2 | B-II |
|---|---|---|
| 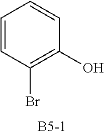 | 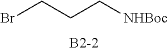 | 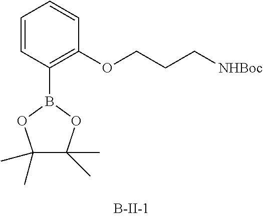 |
| 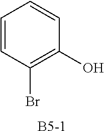 | 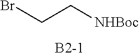 | 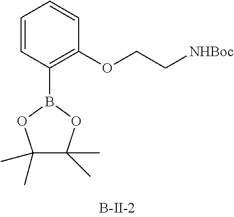 |
| 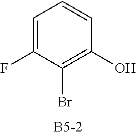 | 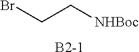 | 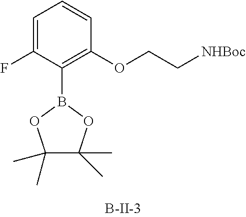 |
| 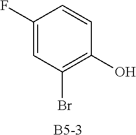 | 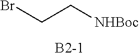 | 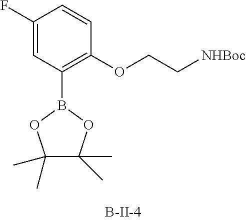 |
| 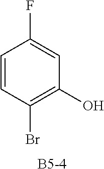 | 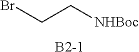 | 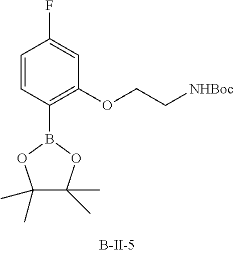 |
| 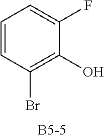 | 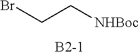 | 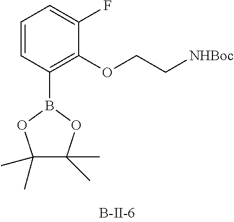 |
| 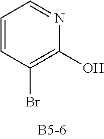 | 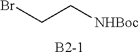 | 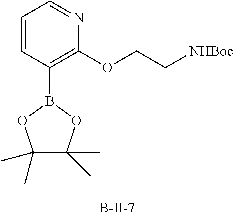 |
| 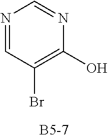 | 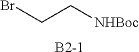 | 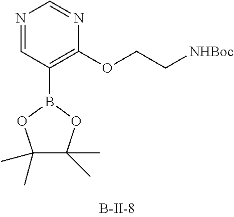 |
| 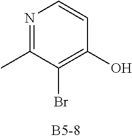 | 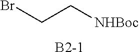 | 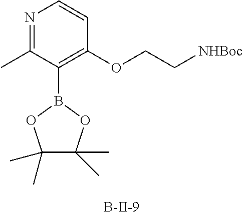 |
| 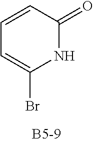 | 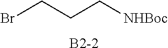 | 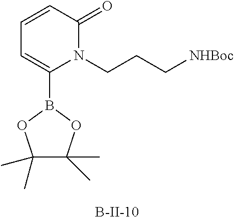 |

General Method B-III

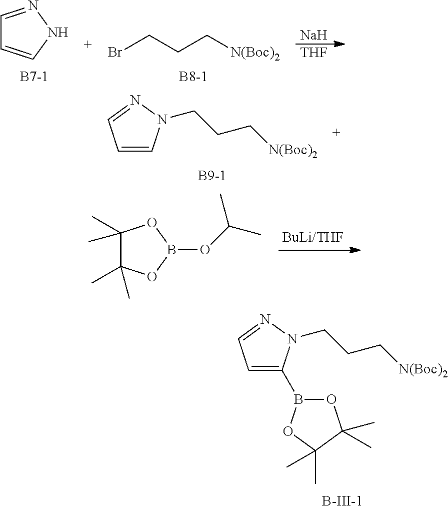

Step 1. 137-1 pyrazole (1.0 eq.) is added to a suspension of NaH (60% in mineral oil. 1.1 eq.) in THF (0.5 M) at ambient temperature. After 30 min, to above suspension is added 138-1 (10 eq). The mixture is stirred at ambient temperature until the reaction is complete, quenched with saturated aqueous ammonium chloride solution, and extracted with EtOAc for three times. The combined extracts are washed with brine, dried over Na₂SO₄, filtered, concentrated and purified on a silica gel column to provide 139-1.

Step 2, To a solution of B9-1 (1.0 eq.) in anhydrous THF (0.2 M) is added n-BuLi (2.5M in hexane, 1.1 eq.) at 0° C. The reaction solution is stirred for 1 hour at ambient temperature and then cooled to −78° C. To the reaction solution is added 2-isopropoxy-4,4,5,5-tetramethyl-1,3,2-dioxaborolane (1.05 eq.). After 15 min at −78° C., the reaction is allowed to warm to 0° C. over 1 hour. The reaction is diluted with saturated NH₄Cl solution and extracted with DCM. The organics are dried over Na₂SO₄, concentrated and purified on a silica gel column to afford B-III-1.

The following pinacol boronates B-III-1-B-III-6 are prepared via the General Method B-III using the corresponding starting materials B7 and B8 as shown in the table below:

| B7 | B8 | B-III |
|---|---|---|
| 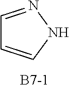 | 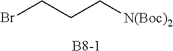 | 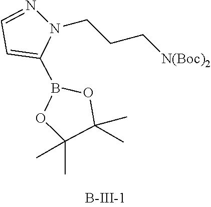 |
| 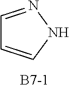 | 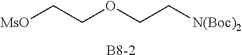 | 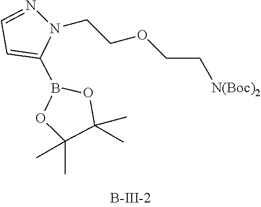 |
| 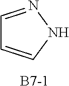 | 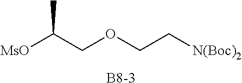 | 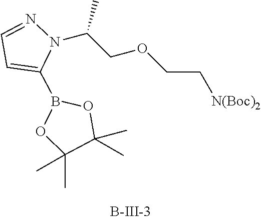 |
| 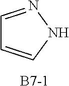 | 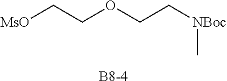 | 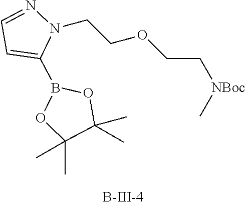 |
| 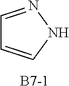 | 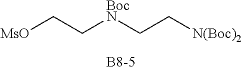 | 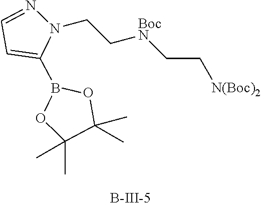 |
| 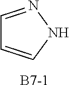 | 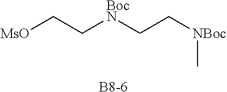 | 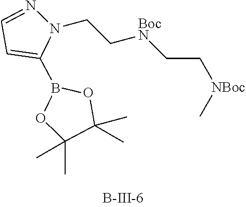 |

General Method B-IV

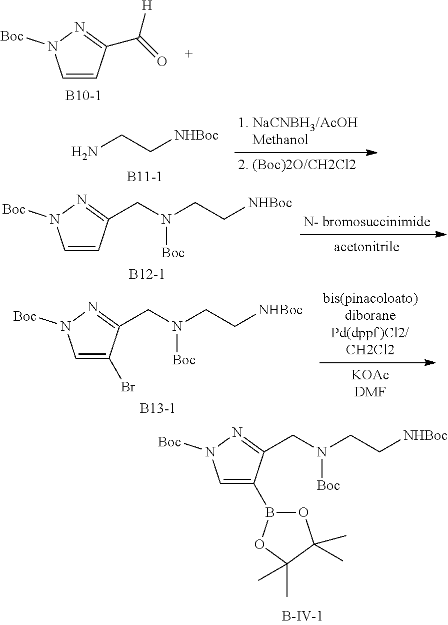

Step 1. To a solution of B10-1 (1 eq.) in methanol (0.2 M) and acetic acid (1.5 eq.) are added B11-1 (1 eq.) and NaCNBH₃ (2 eq.) at ambient temperature. The mixture is stirred for 1 hour and partitioned between water and ethyl acetate. The organic phase layer is separated, washed sequentially with saturated NaHCO₃ and brine, concentrated and dried under vacuum. The residue is dissolved in CH₂Cl₂ (0.2 M) and the solution is cooled to 0° C. To the solution is added di(tert-butyl) dicarbonate (1.2 eq) portionwise. The ice bath is removed, and the mixture is stirred for overnight at ambient temperature. The reaction solution is diluted with dichloromethane, washed with water, and dried over magnesium sulfate. After filtration and condensation, the residue is purified on a silica gel column to provide B12-1.

Step 2 and Step 3 are the same as Step 2 and Step 3 in General Method B-I to provide B-IV-1.

The following pinacol boronates B-IV-1-B-IV-7 are prepared via the General Method B-IV using the corresponding starting materials B10 and B11 as shown in the table below:

| B10 | B11 | B-IV |
|---|---|---|
| 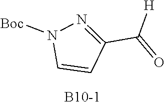 | 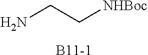 | 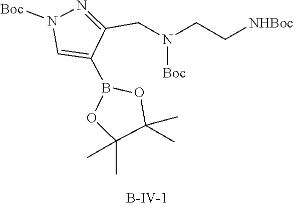 |
| 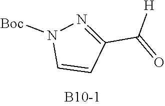 | 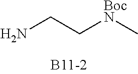 | 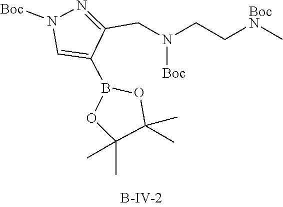 |
| 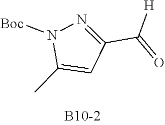 | 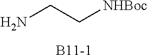 | 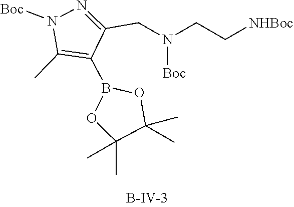 |
| 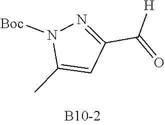 | 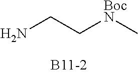 | 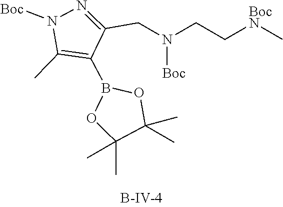 |
| 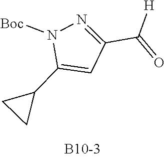 | 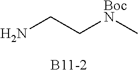 | 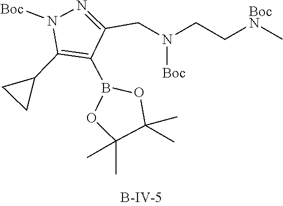 |
| 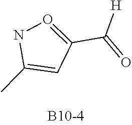 | 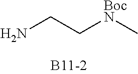 | 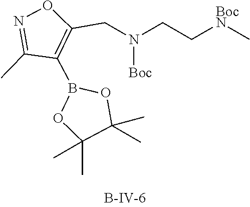 |
| 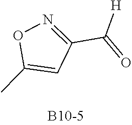 | 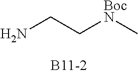 | 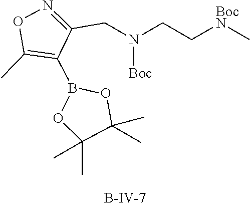 |

General Method C

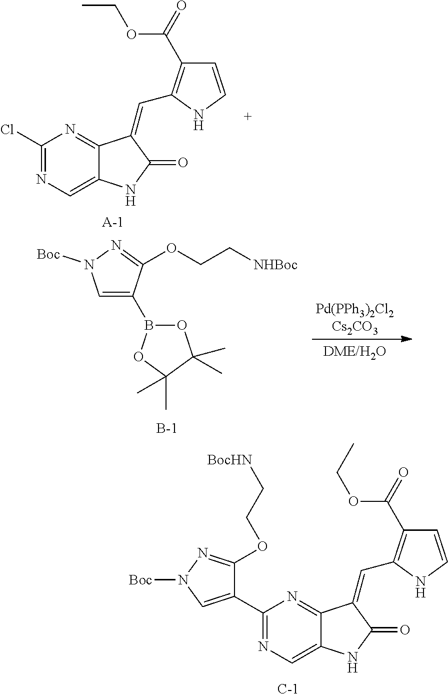

To a solution of A-1 (1.0 eq.) and B-1 (1.2 eq.) and Cs₂CO₃ (3 eq.) in DME/H₂O (5:1, 0.2 M) under N₂, is added Pd(PPh₃)₂Cl₂ (0.05 eq.). The mixture is stirred at 85° C. overnight, cooled to ambient temperature, and quenched with H₂O. The resulting mixture is extracted with EtOAc for three times. The combined extracts are washed with brine and dried over anhydrous Na₂SO₄. After filtration and condensation, the resulting residue is purified by a silica gel column chromatography to afford the desired product C-1.

The following intermediates C-1-C-66 are prepared via the General Method C using the corresponding two starting materials A and B as shown in the table below:

| A | B | C |
|---|---|---|
| 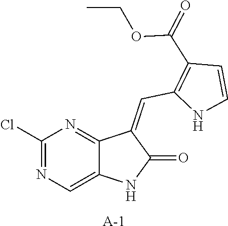 | 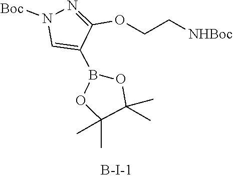 | 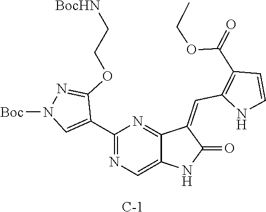 |
| 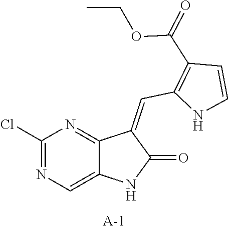 | 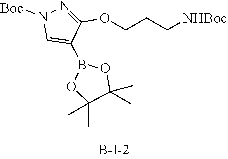 | 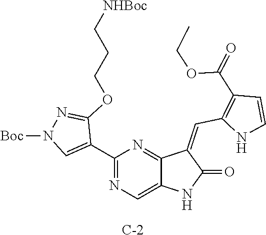 |
| 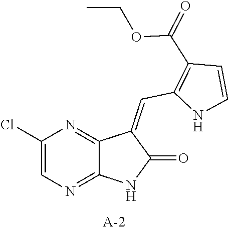 | 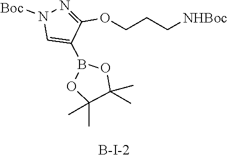 | 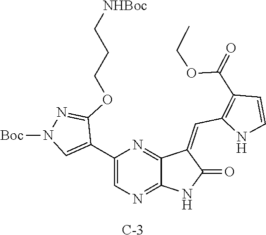 |
| 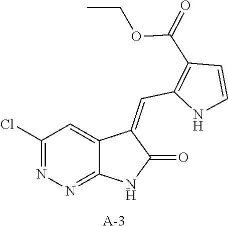 | 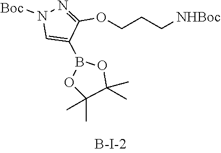 | 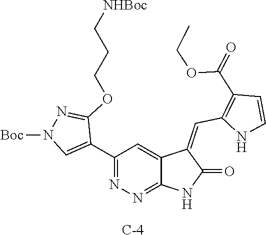 |
| 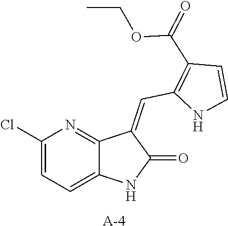 | 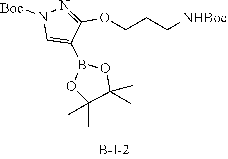 | 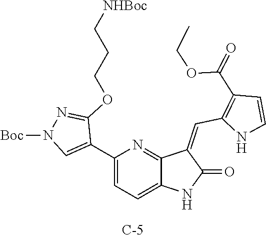 |
| 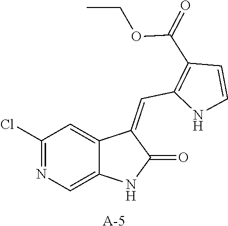 | 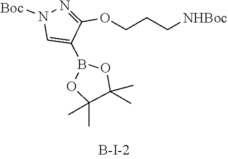 | 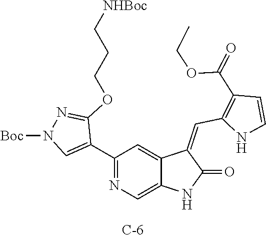 |
| 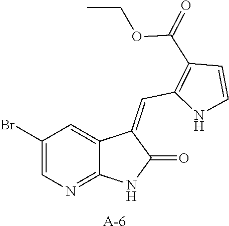 | 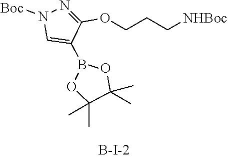 | 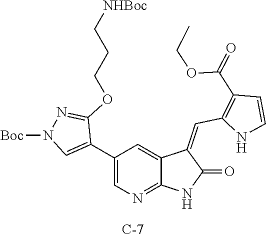 |
| 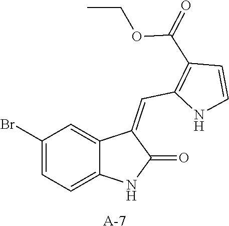 | 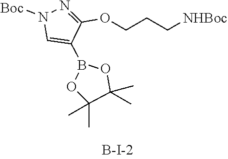 | 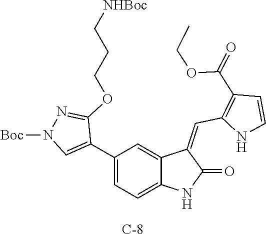 |
| 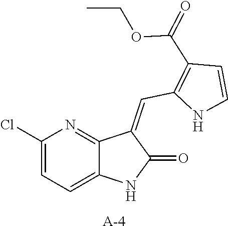 | 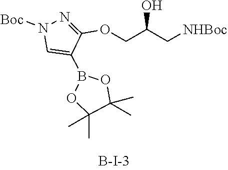 | 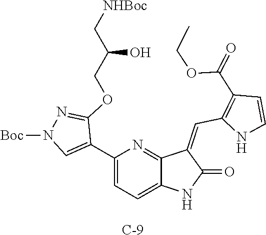 |
| 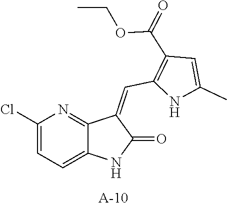 | 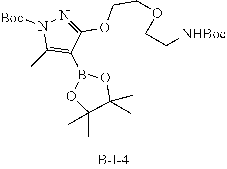 | 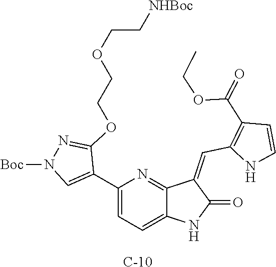 |
| 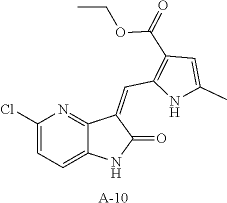 | 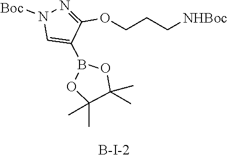 | 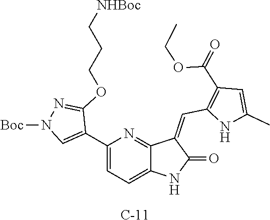 |
| 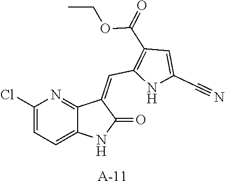 | 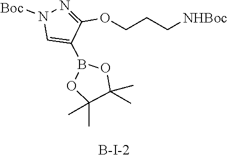 | 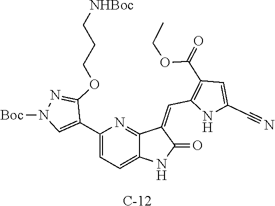 |
| 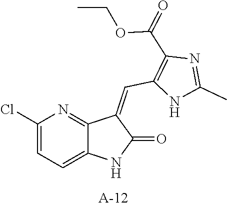 | 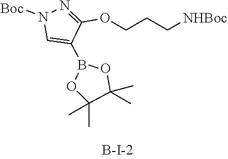 | 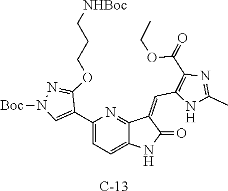 |
| 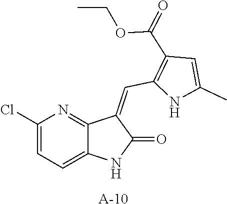 | 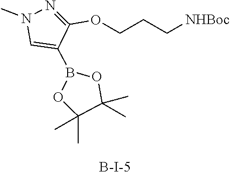 | 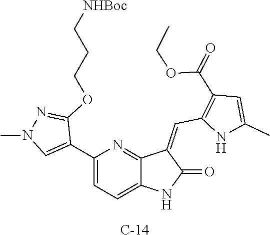 |
| 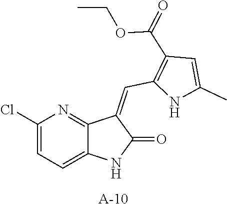 | 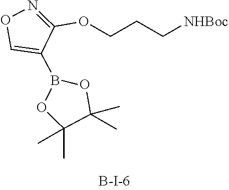 | 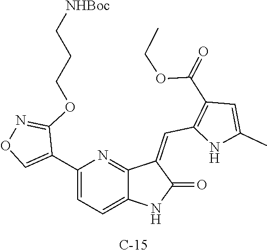 |
| 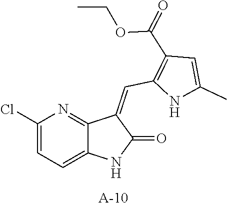 | 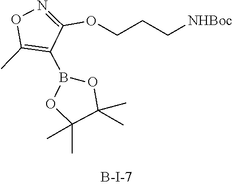 | 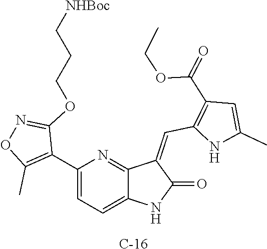 |
| 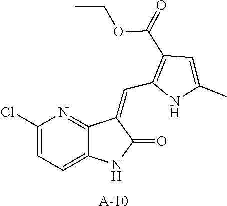 | 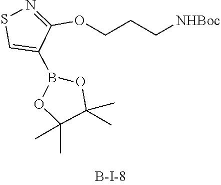 | 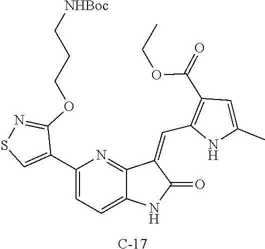 |
| 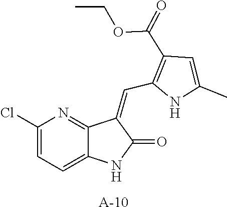 | 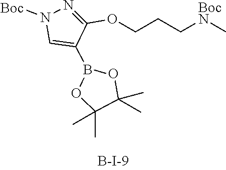 | 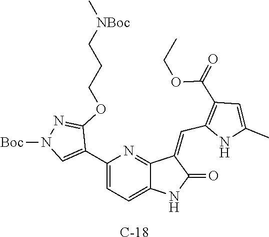 |
| 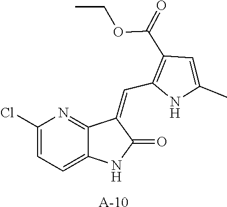 | 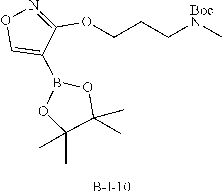 | 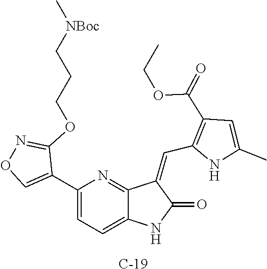 |
| 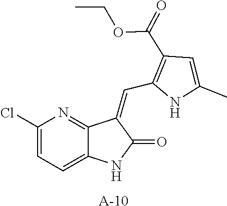 | 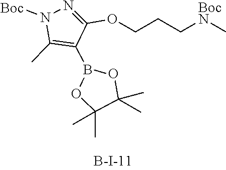 | 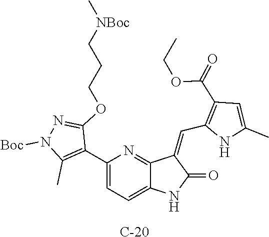 |
| 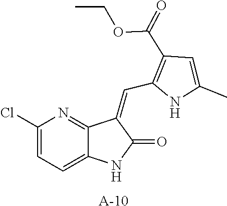 | 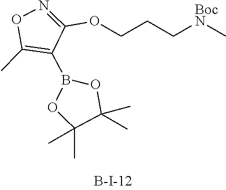 | 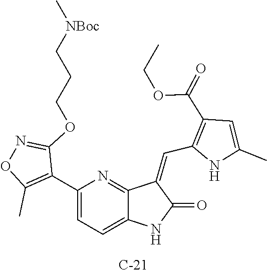 |
| 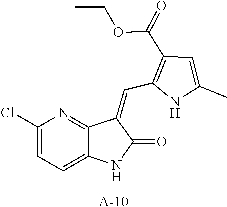 | 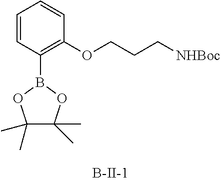 | 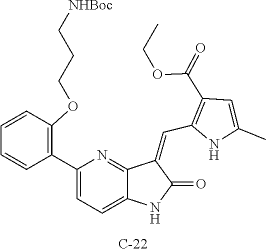 |
| 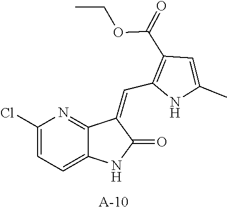 | 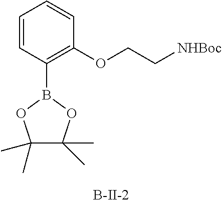 | 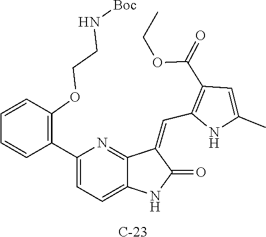 |
| 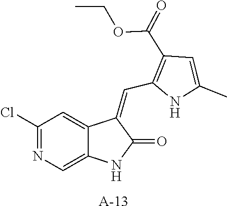 | 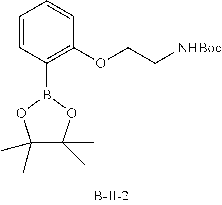 | 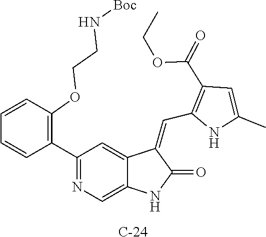 |
| 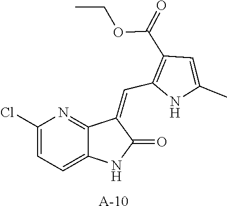 | 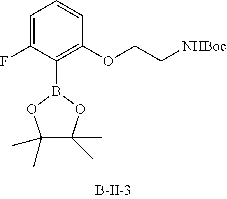 | 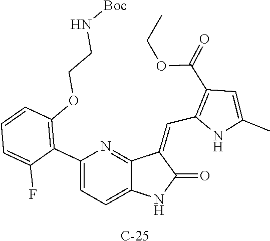 |
| 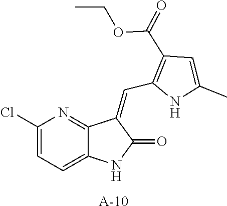 | 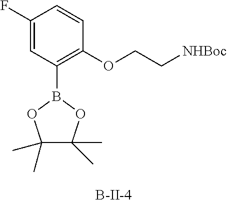 | 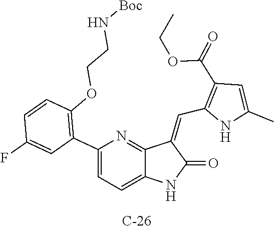 |
| 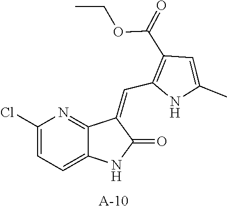 | 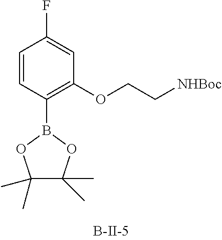 | 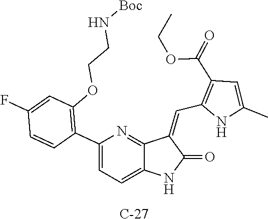 |
| 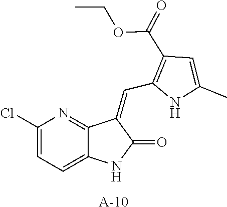 | 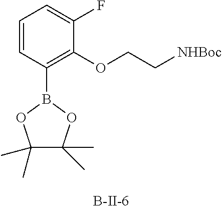 | 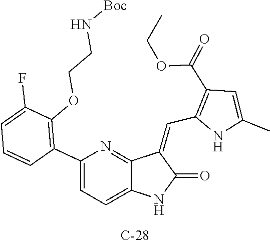 |
| 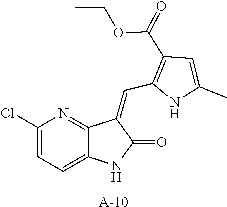 | 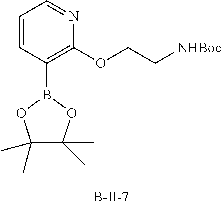 | 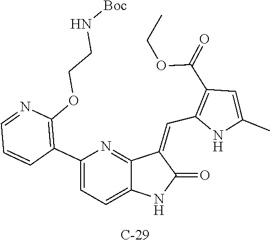 |
| 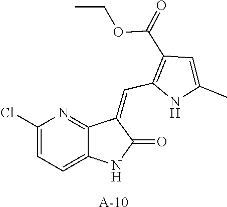 | 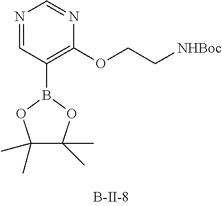 | 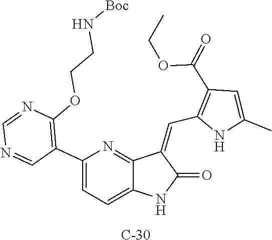 |
| 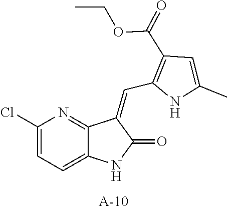 | 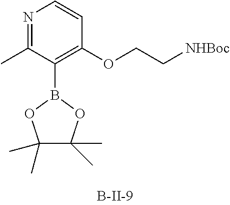 | 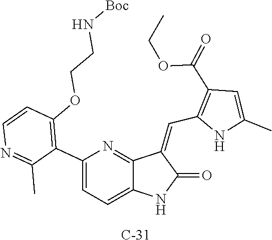 |
| 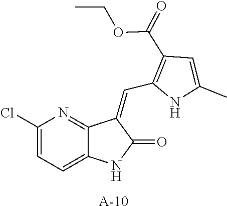 | 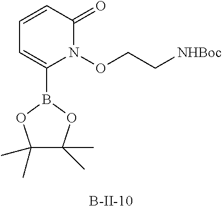 | 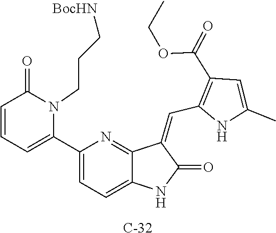 |
| 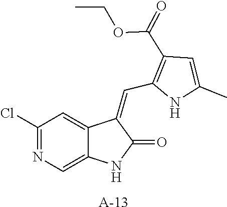 | 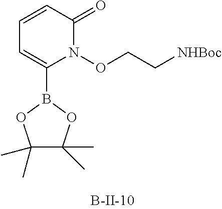 | 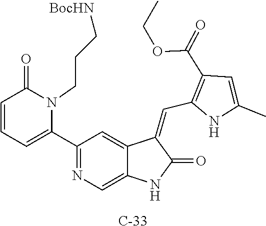 |
| 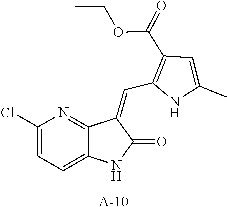 | 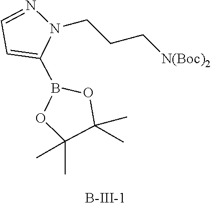 | 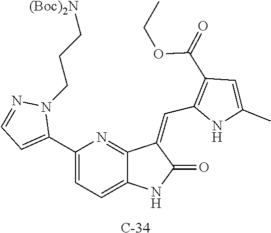 |
| 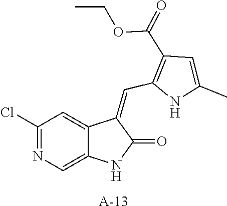 | 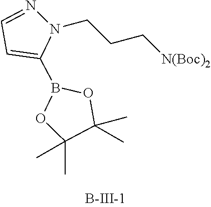 | 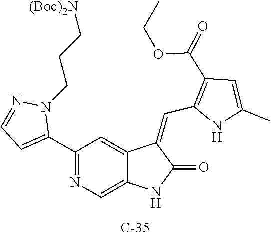 |
| 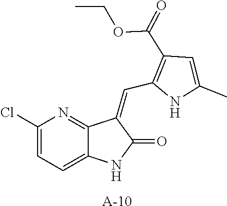 | 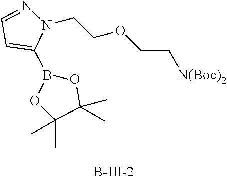 | 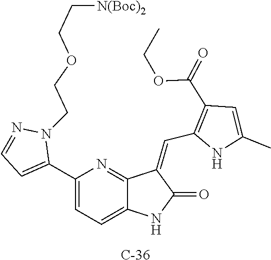 |
| 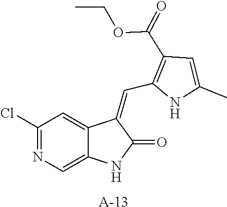 | 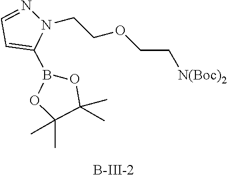 | 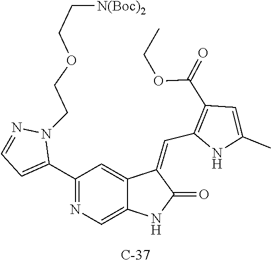 |
| 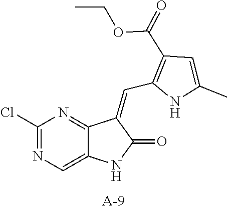 | 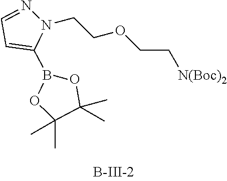 | 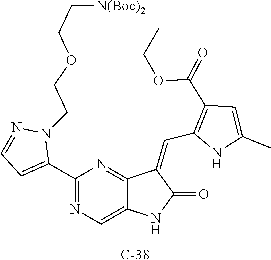 |
| 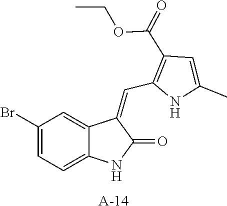 | 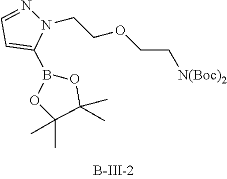 | 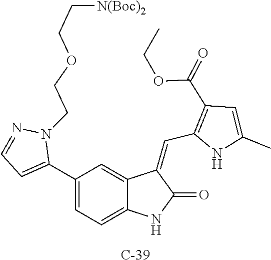 |
| 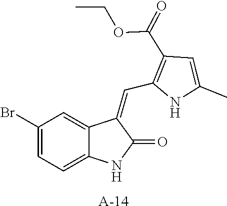 | 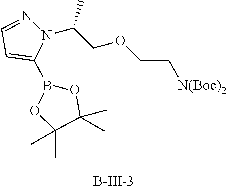 | 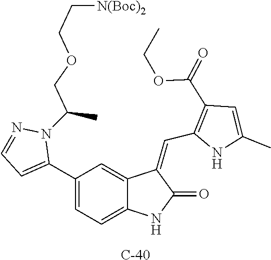 |
| 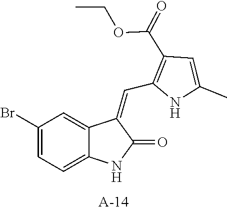 | 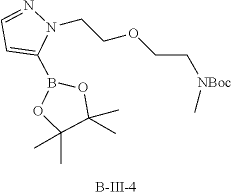 | 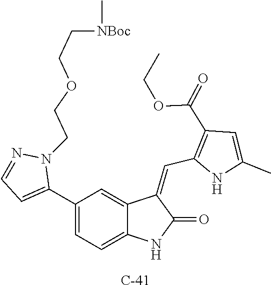 |
| 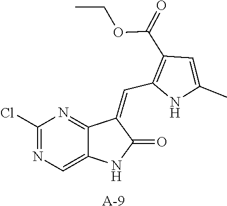 | 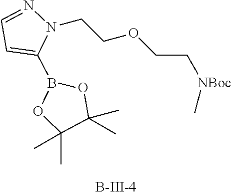 | 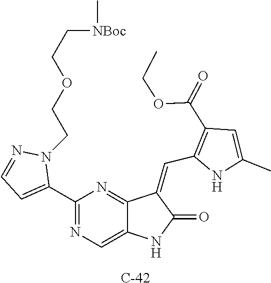 |
| 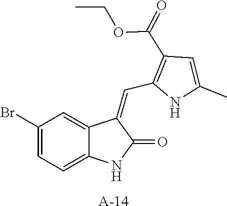 | 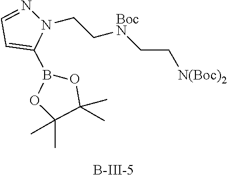 | 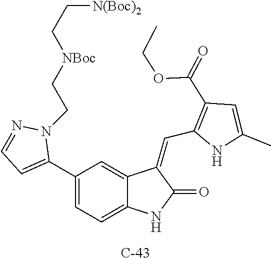 |
| 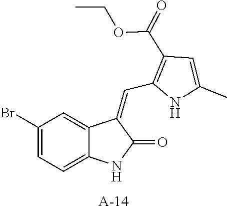 | 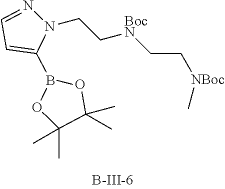 | 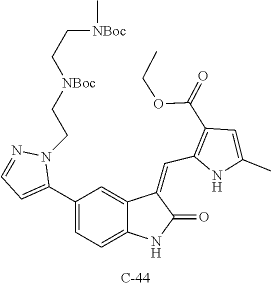 |
| 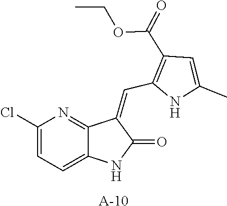 | 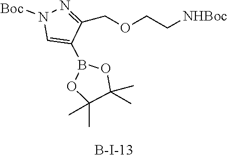 | 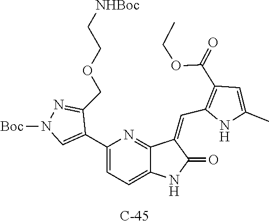 |
| 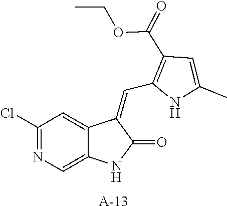 | 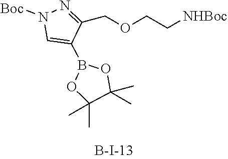 | 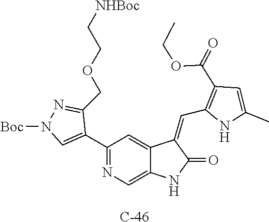 |
| 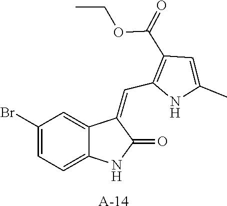 | 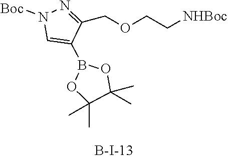 | 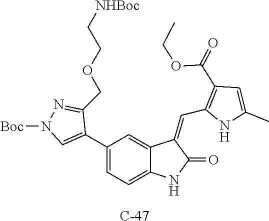 |
| 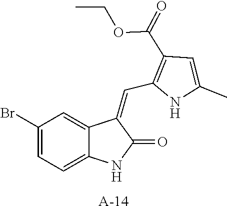 | 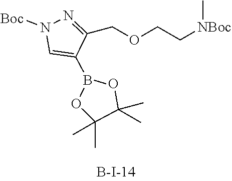 | 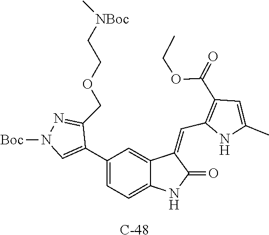 |
| 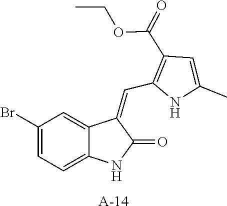 | 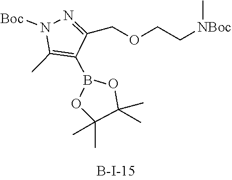 | 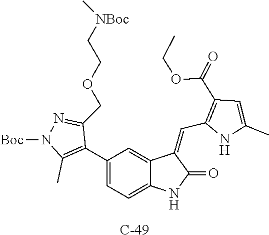 |
| 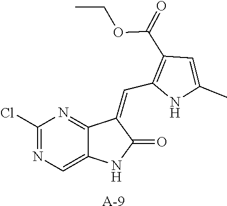 | 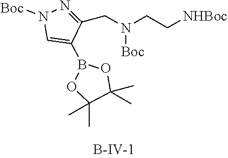 | 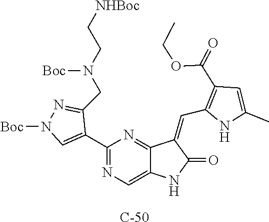 |
| 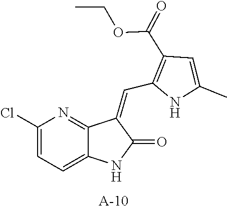 | 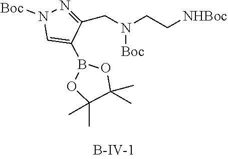 | 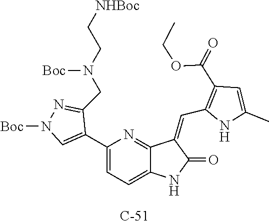 |
| 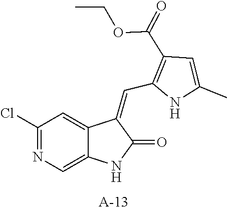 | 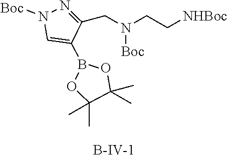 | 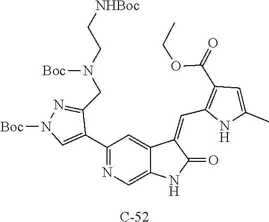 |
| 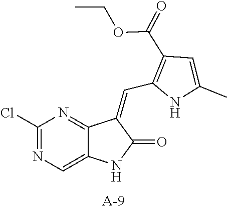 | 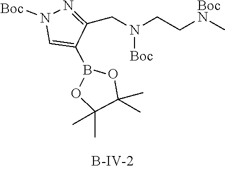 | 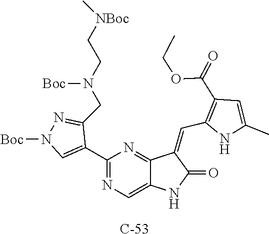 |
| 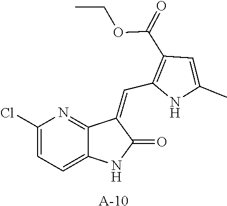 | 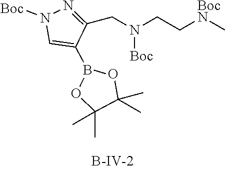 | 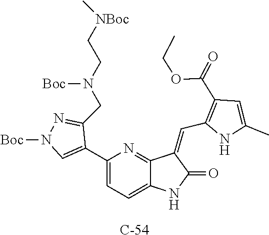 |
| 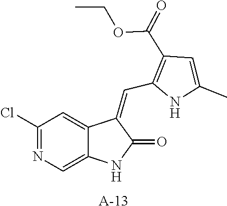 | 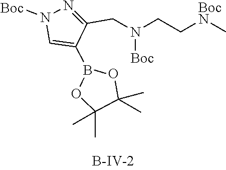 | 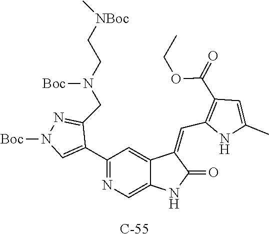 |
| 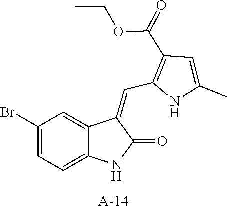 | 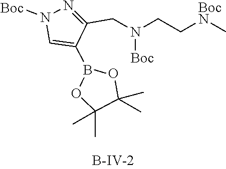 | 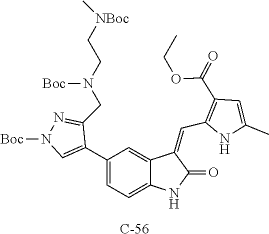 |
| 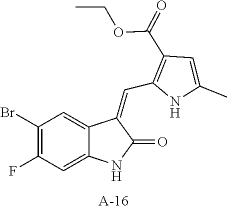 | 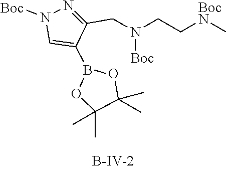 | 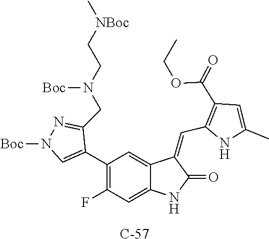 |
| 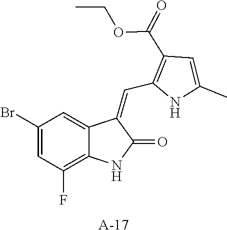 | 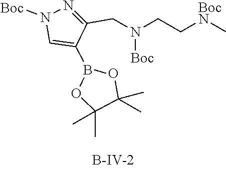 | 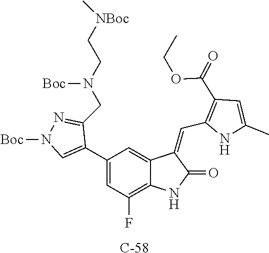 |
| 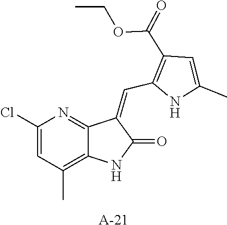 | 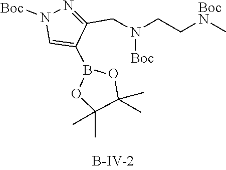 | 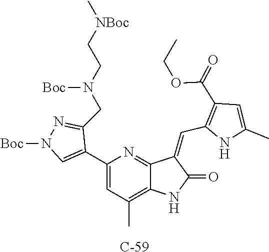 |
| 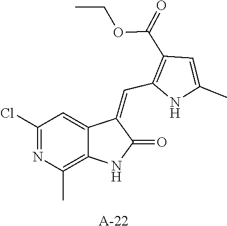 | 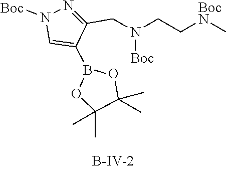 | 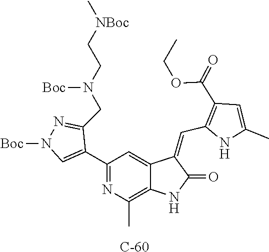 |
| 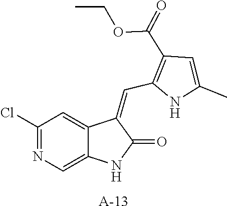 | 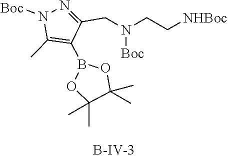 | 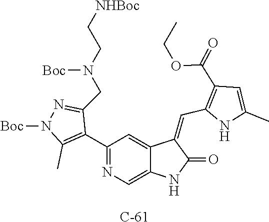 |
| 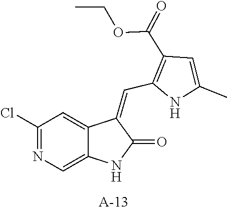 | 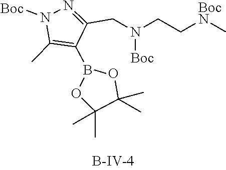 | 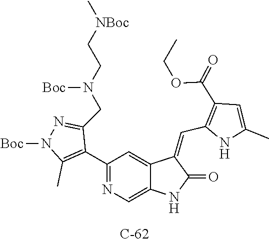 |
| 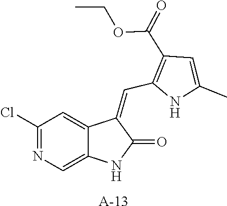 | 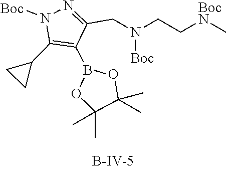 | 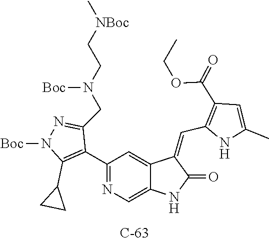 |
| 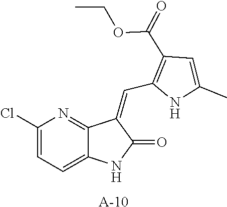 | 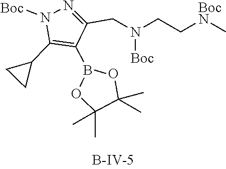 | 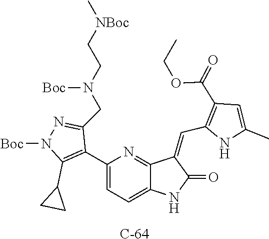 |
| 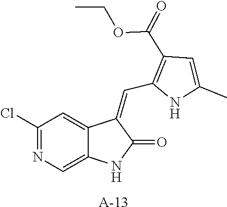 | 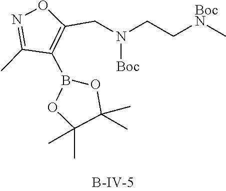 | 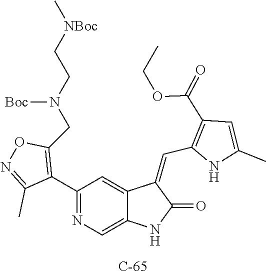 |
| 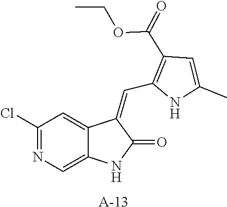 | 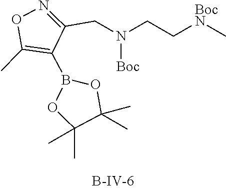 | 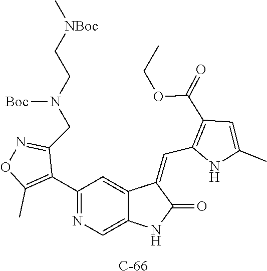 |

General Method D

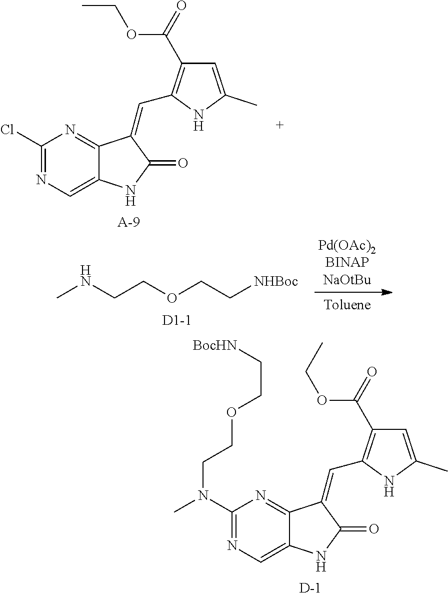

To a stirring solution of A-9 (1.0 eq.) in toluene (0.2 M) are added D1-1 (1.5 eq.) and sodium tert-butoxide (3 eq.), BINAP (0.05 eq.) and Pd(OAc)₂ (0.05) under nitrogen. The mixture is heated at 85° C. for 20 h and cooled to ambient temperature. The reaction is quenched with sat. aqueous ammonium chloride and extracted with EtOAc. The combined extracts are washed with brine and dried over Na₂SO₄ After filtration and concentration, the residue is purified on a silica gel column to provide D-1.

The following intermediates D-1-D-16 are prepared via the General Method D using the corresponding two starting materials A and D1 as shown in the table below:

| A | D1 | D |
|---|---|---|
| 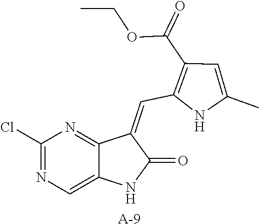 | 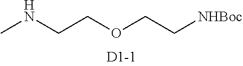 | 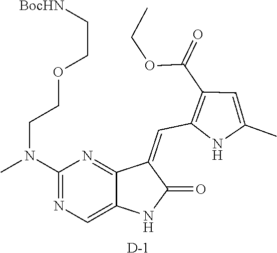 |
| 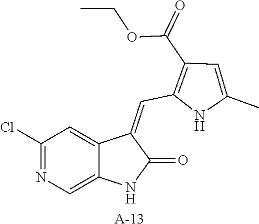 | 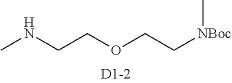 | 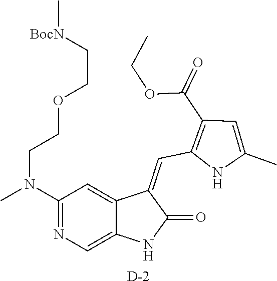 |
| 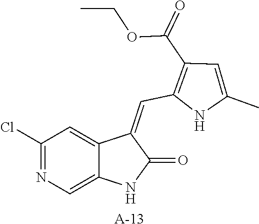 | 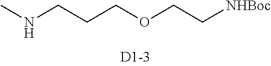 | 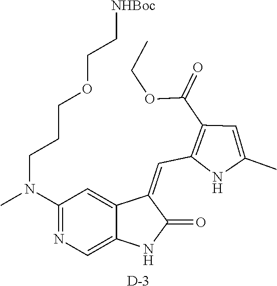 |
| 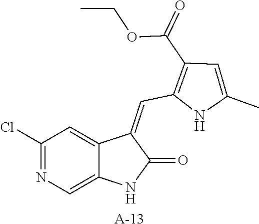 | 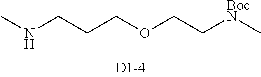 | 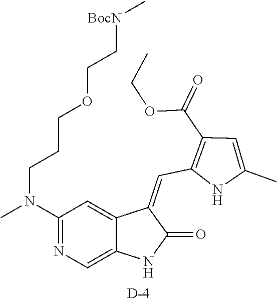 |
| 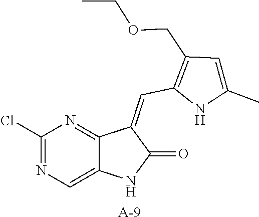 | 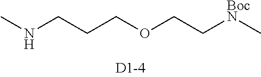 | 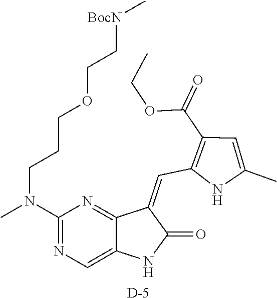 |
| 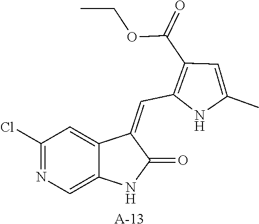 | 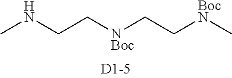 | 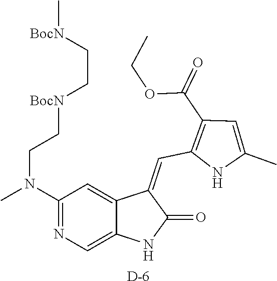 |
| 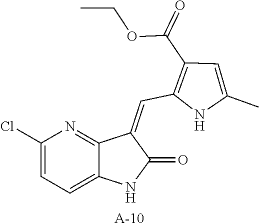 | 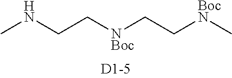 | 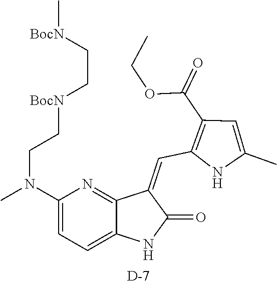 |
| 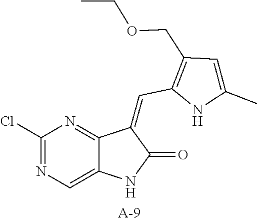 | 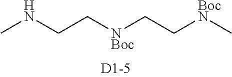 | 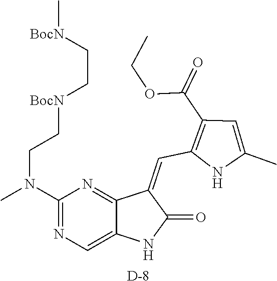 |
| 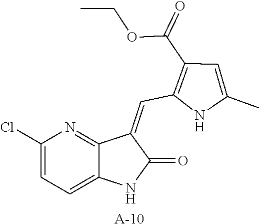 | 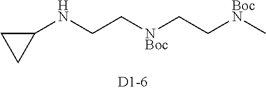 | 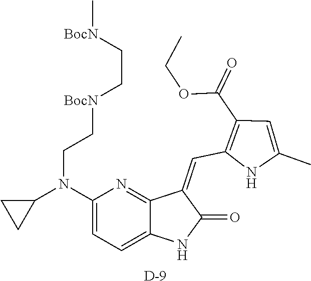 |
| 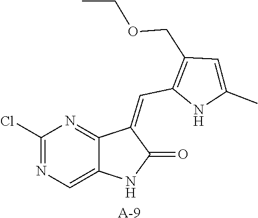 | 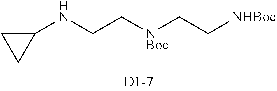 | 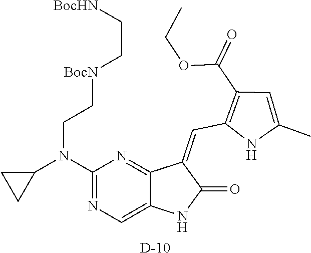 |
| 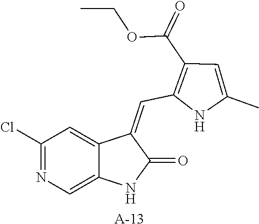 | 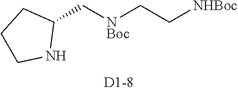 | 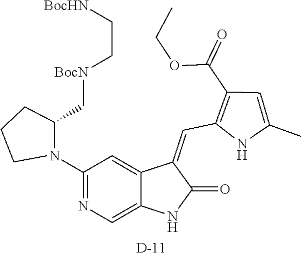 |
| 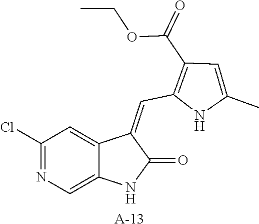 | 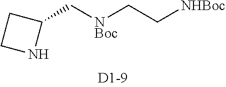 | 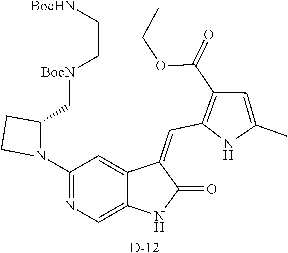 |
| 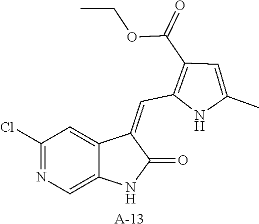 | 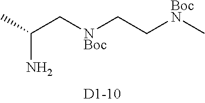 | 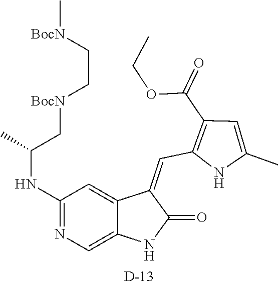 |
| 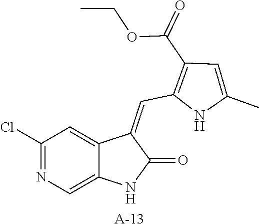 | 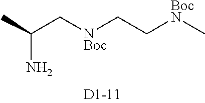 | 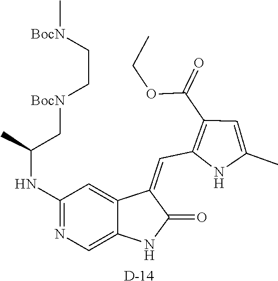 |
| 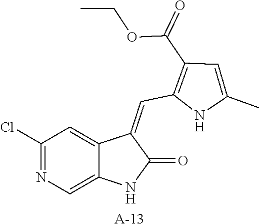 | 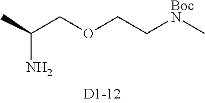 | 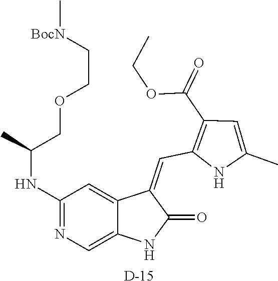 |
| 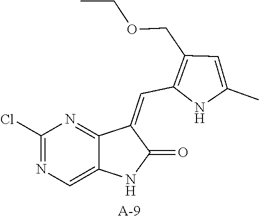 | 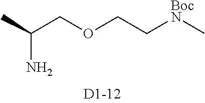 | 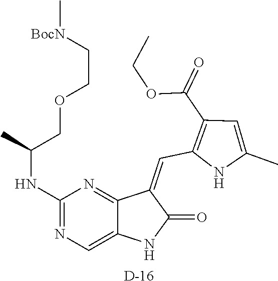 |

General Method E

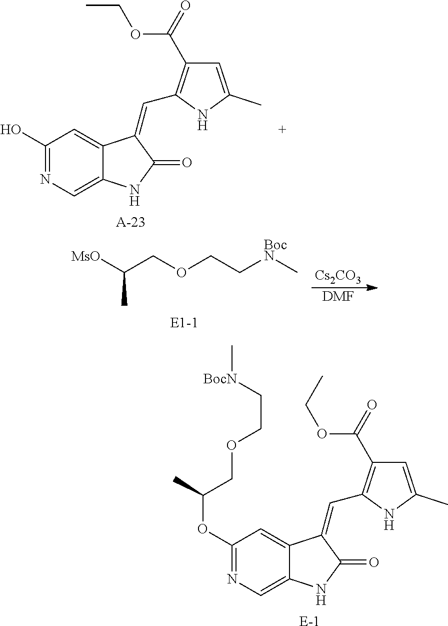

To a solution of A-23 (1.0 eq.) and E1-1 (1.5 equivalent) in DMF (0.25 M) is added Cs₂CO₃ (2.0 eq.) and the mixture is heated at 60-80° C. under nitrogen until the reaction is completed. Water (5 volume of DMF) is added to the cooled DMF solution and the product is extracted with ethyl acetate (1 volume of water) for three times. The combined extracts are washed with water, aqueous HCl solution (1 N), brine, and dried over magnesium sulfate. After filtration and condensation, the crude product is purified on a silica gel column to provide pure product E-1.

The following intermediates E-1-E-16 are prepared via the General Method E using the corresponding two starting materials A and E1 as shown in the table below:

| A | E1 | E |
|---|---|---|
| 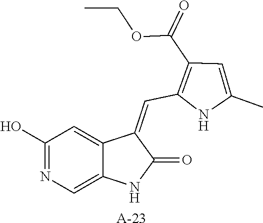 | 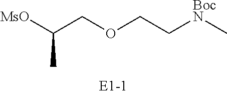 | 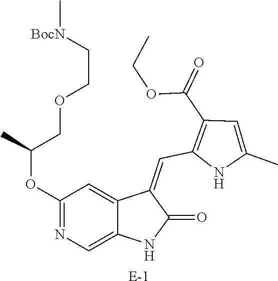 |
| 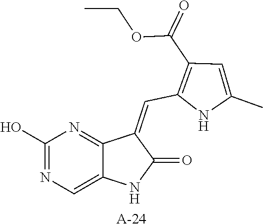 | 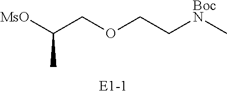 | 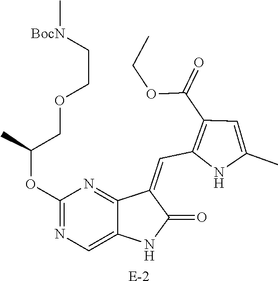 |
| 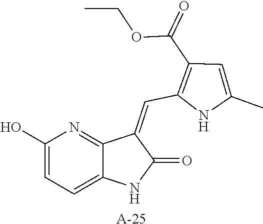 | 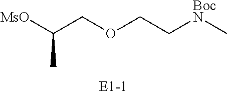 | 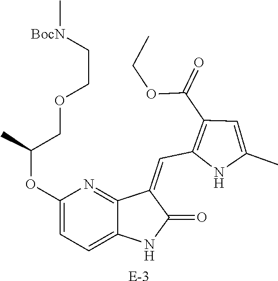 |
| 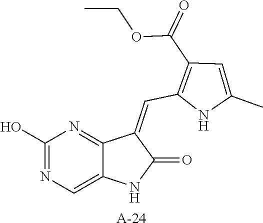 | 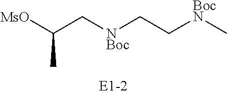 | 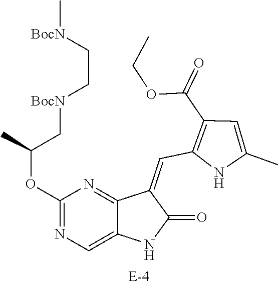 |
| 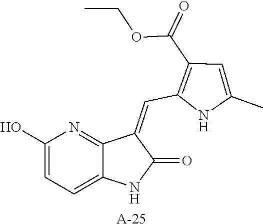 | 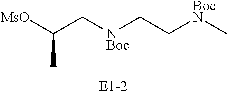 | 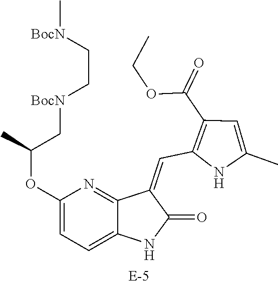 |
| 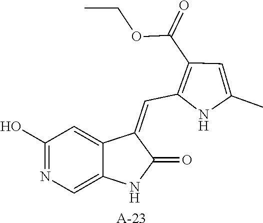 | 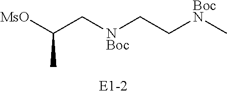 | 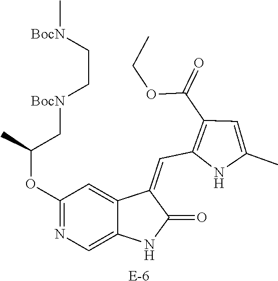 |
| 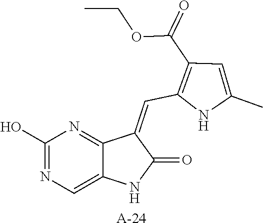 | 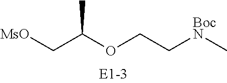 | 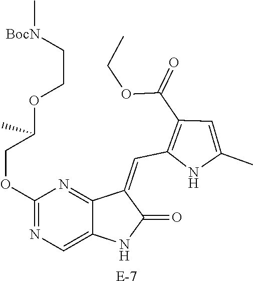 |
| 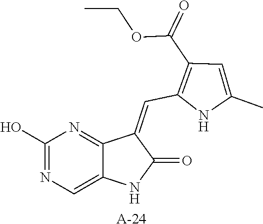 | 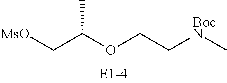 | 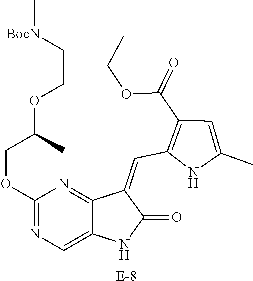 |
| 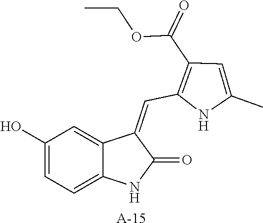 | 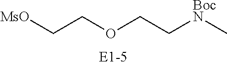 | 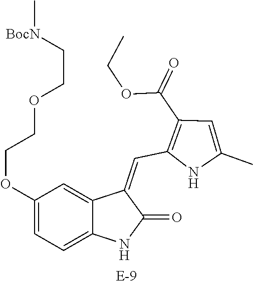 |
| 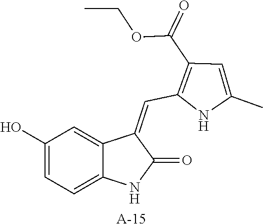 | 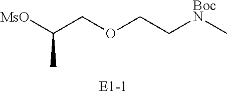 | 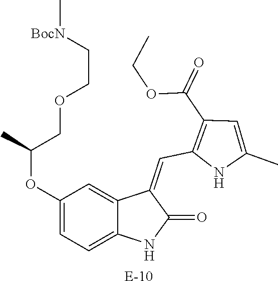 |
| 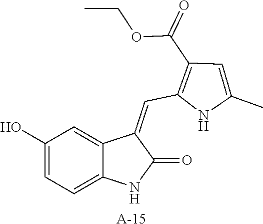 | 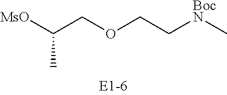 | 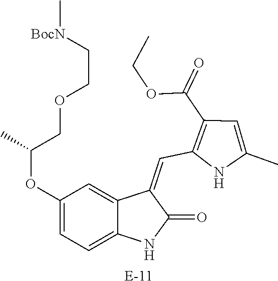 |
| 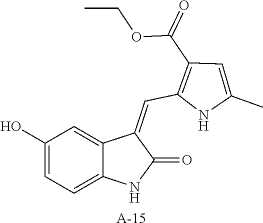 | 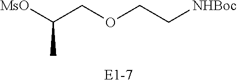 | 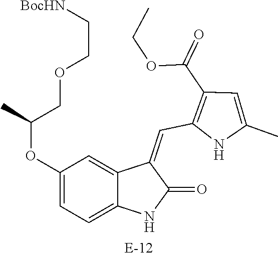 |
| 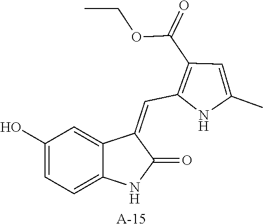 | 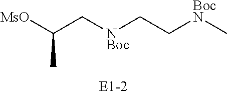 | 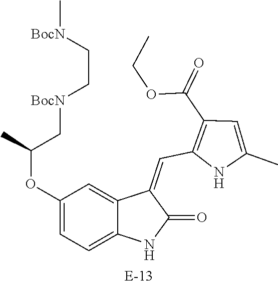 |
| 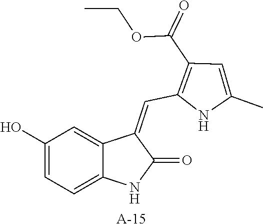 | 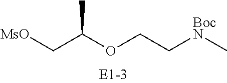 | 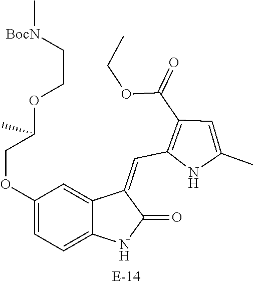 |
| 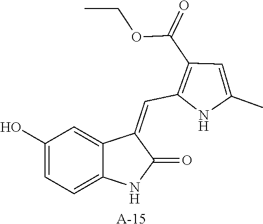 | 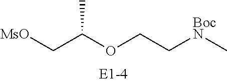 | 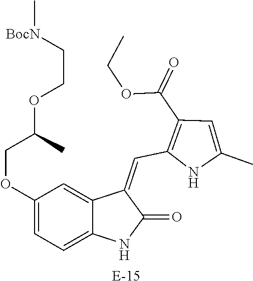 |
| 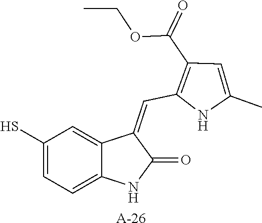 | 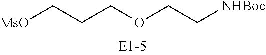 | 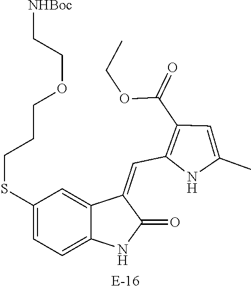 |

General Method F

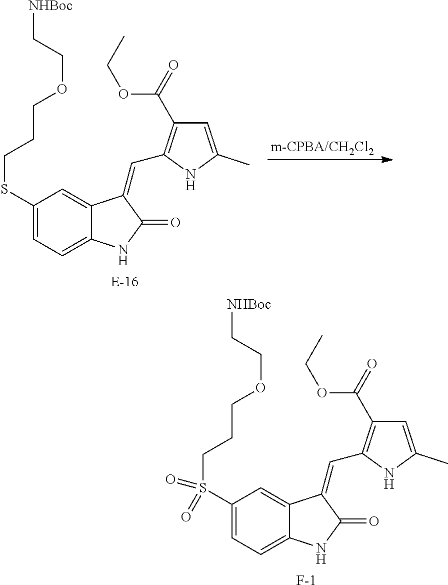

To a solution of E-16 (1.0 eq.) in DCM (0.2 M) is added m-chloroperbenzoic acid (m-CPBA) (3 eq.) at 0° C. The reaction mixture is allowed to warm to ambient temperature and stirred for 4 h. The mixture is quenched with aqueous sodium thiosulfate (1 M) and extracted with DCM. The combined extracts are washed with brine and dried over sodium sulfate. After filtration and condensation, the residue is purified by flash column chromatography on silica gel to afford F-1.

General Method G

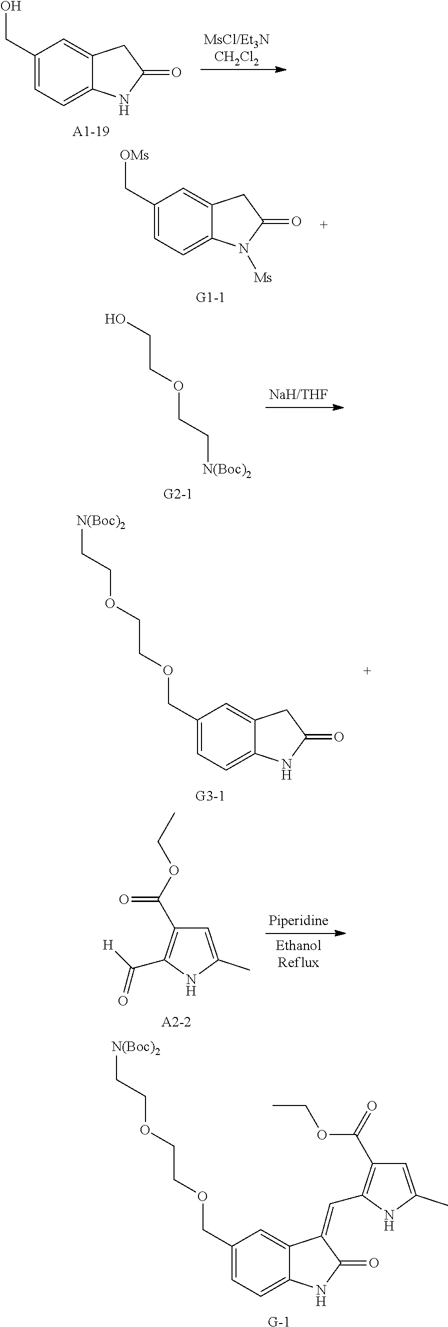

Step 1. To a solution of A1-19 (1.0 eq.) in DCM (0.2 M) and Et₃N (4 eq.) with iced bath is added MsCl (3 eq.) and the mixture is stirred overnight from 0° C. to ambient temperature. The reaction is diluted with DCM, washed with ice water and brine, and dried over Na₂SO₄. After filtration and condensation, the residue is dried with vacuum to provide G1-1 which is used without further purification.

Step 2. G2-1 (1.0 eq.) is added to a solution of NaH (60% in mineral oil, 1.2 eq.) in anhydrous THF (0.5 M) at ambient temperature. After 30 min, to above suspension is added G1-1 (1.0 eq)v After the reaction is complete, the reaction is quenched with saturated aqueous ammonium chloride solution and extracted with EtOAc for three times. The combined extracts are washed with brine, dried over Na₂SO₄, filtered, concentrated and dried under a vacuum. The residue is dissolved in THF/water (1:1, 0.5 NM) and to the mixture is added aqueous NaOH (6M, 3 eq.). The resulting mixture is stirred at 60‘C’ until the hydrolysis is complete. The reaction solution is cooled to ambient temperature, diluted with EtOAC, washed with brine, and dried over Na₂SO₄. After filtration and condensation, the residue is purified by a silica gel column to provide G3-1.

Step 3. G3-7_reacts with A2-2 to provide G-1 following the General Procedure A.

The following intermediates G-1-G-4 are prepared via the General Method G using the corresponding two starting materials A1 and G2 as shown in the table below:

| A1 | G2 | G |
|---|---|---|
| 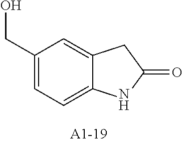 | 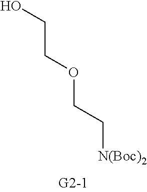 | 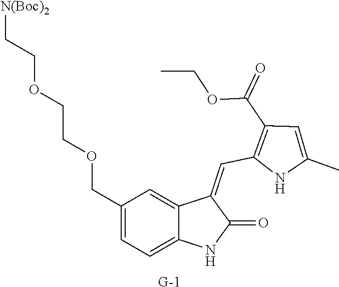 |
| 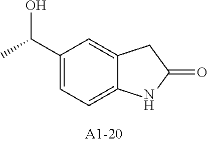 | 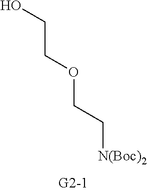 | 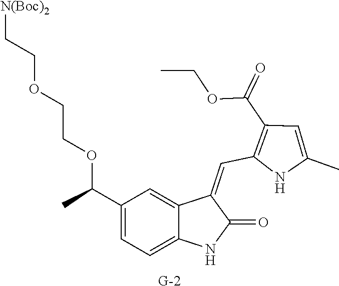 |
| 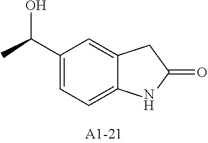 | 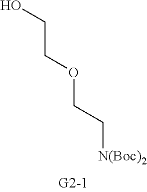 | 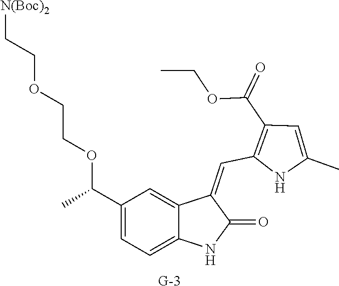 |
| 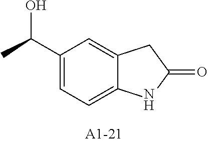 | 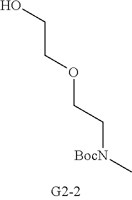 | 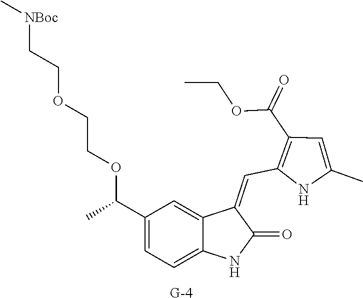 |

General Method 1H

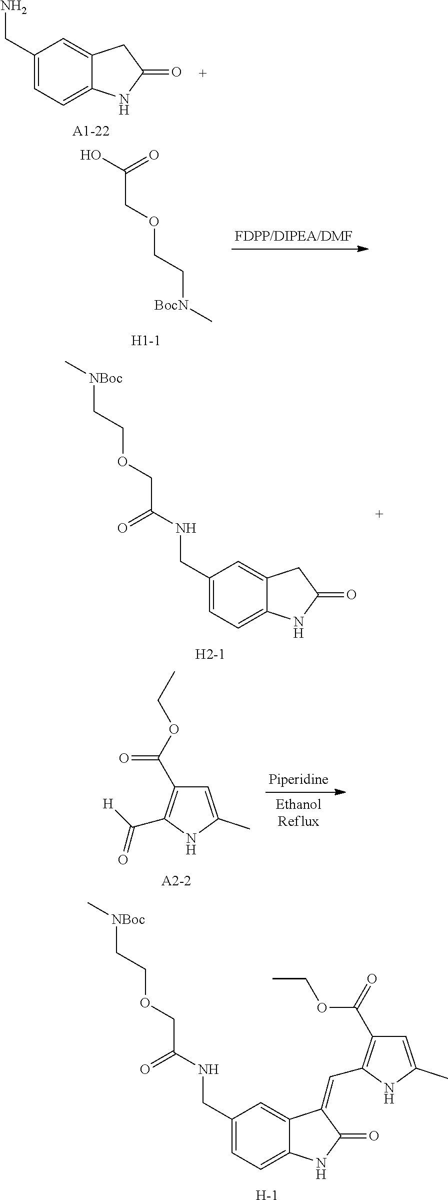

Step 1. To a solution of A1-22 (1.0 eq.) and H10 (1.0 eq.) in DMF (0.2 M) are added DIPEA (3 eq.) and pentafluorophenyl diphenylphosphinate (FDPP) (1.1 eq). The solution is stirred at ambient temperature until the amide formation is completed. The mixture is diluted with water and extracted with EtOAc for three times. The combined extracts are washed with water for three times, aqueous HCl (1N), saturated aqueous Na₂CO₃ and brine, dried over Na₂SO₄, and concentrated. The resulting residue is purified by a silica gel column to afford H2-1.

Step 2. H2-1 reacts with A2-2 to provide H-1 following the General Procedure A.

The following intermediates H-1-H-10 are prepared via the General Method H using the corresponding two starting materials A1 and H1 as shown in the table below:

| A1 | H1 or D1 | H |
|---|---|---|
| 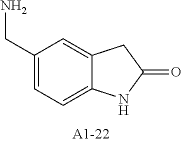 | 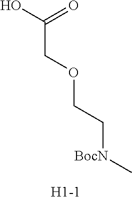 [SMILES: CN(CCOCC(=O)O)C(=O)OC(C)(C)C] | 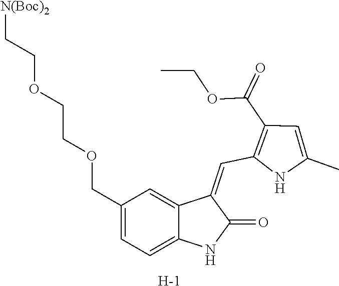 |
| 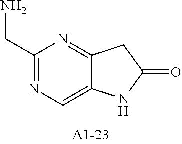 | 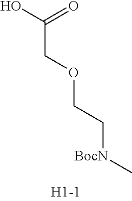 [SMILES: CN(CCOCC(=O)O)C(=O)OC(C)(C)C] | 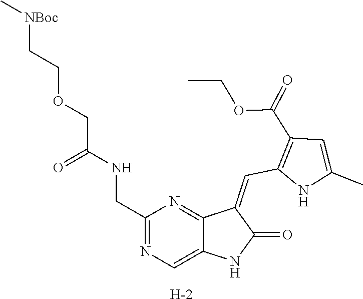 |
| 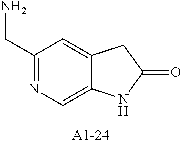 | 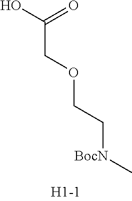 [SMILES: CN(CCOCC(=O)O)C(=O)OC(C)(C)C] | 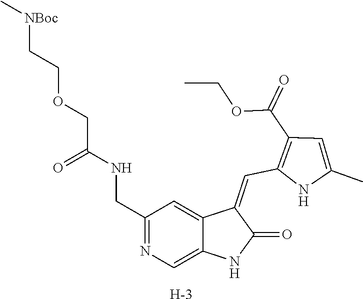 |
| 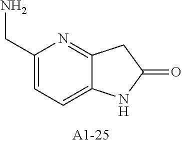 | 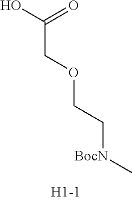 [SMILES: CN(CCOCC(=O)O)C(=O)OC(C)(C)C] | 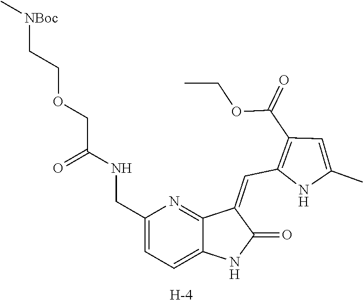 |
| 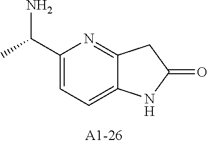 | 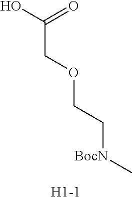 [SMILES: CN(CCOCC(=O)O)C(=O)OC(C)(C)C] | 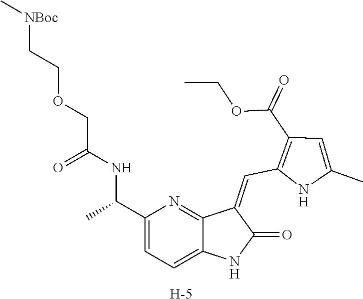 |
| 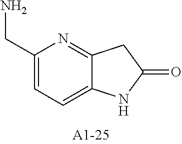 | 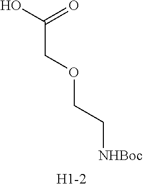 [SMILES: CC(C)(C)OC(=O)NCCOCC(=O)O] | 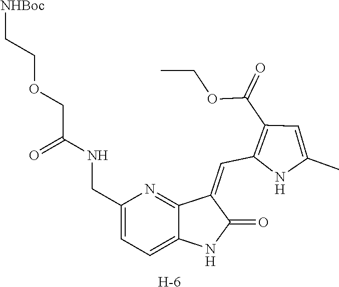 |
| 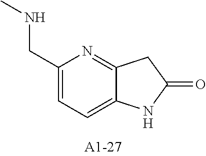 | 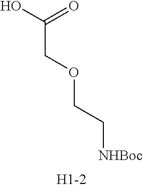 [SMILES: CC(C)(C)OC(=O)NCCOCC(=O)O] | 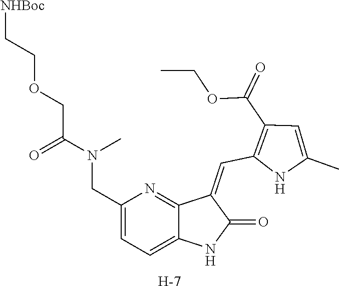 |
| 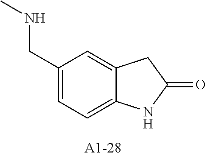 | 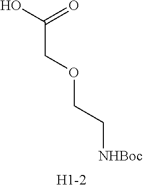 [SMILES: CC(C)(C)OC(=O)NCCOCC(=O)O] | 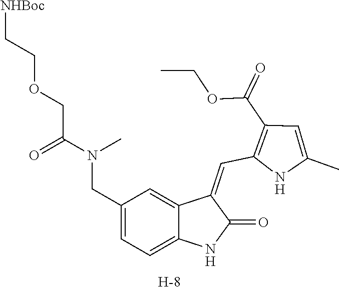 |
| 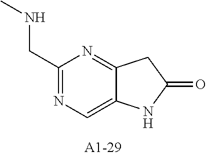 | 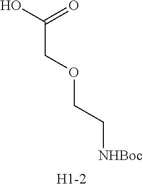 [SMILES: CC(C)(C)OC(=O)NCCOCC(=O)O] | 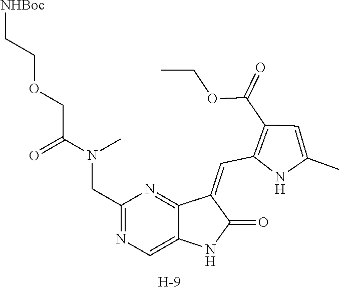 |
| 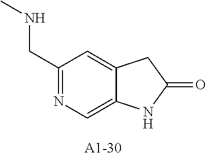 | 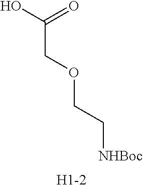 [SMILES: CC(C)(C)OC(=O)NCCOCC(=O)O] | 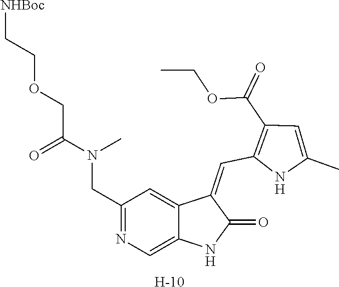 |

General Method I

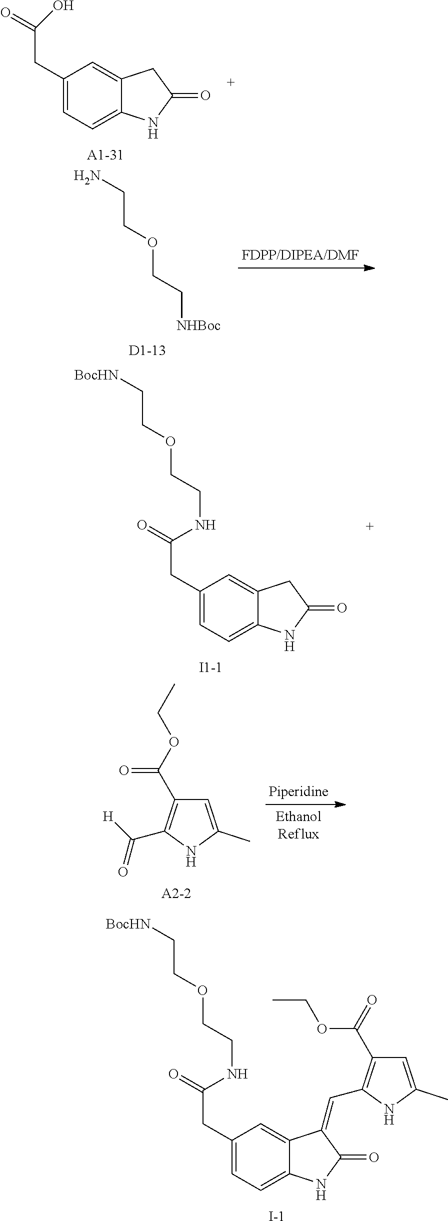

Step 1. To a solution of A1-31 (1.0 eq.) and D1-13 (1.0 eq.) in DMF (0.2 M) are added DIPEA (3 eq.) and pentafluorophenyl diphenylphosphinate (FDPP) (1.1 eq). The solution is stirred at ambient temperature until the amide formation is completed. The mixture is diluted with water and extracted with EtOAc for three times. The combined extracts are washed with water for three times, aqueous HCl (1N), saturated aqueous Na₂CO₃ and brine, dried over Na₂SO₄, and concentrated. The resulting residue is purified by a silica gel column to afford I1-1.

Step 2. I1-1 reacts with A2-2 to provide I-1 following the General Procedure A.

The following intermediates I-1-I-5 are prepared via the General Method I using the corresponding two starting materials A1 and D1 as shown in the table below:

| A1 | D1 | I |
|---|---|---|
| 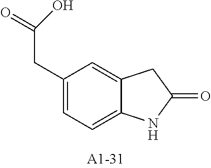 | 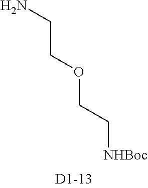 | 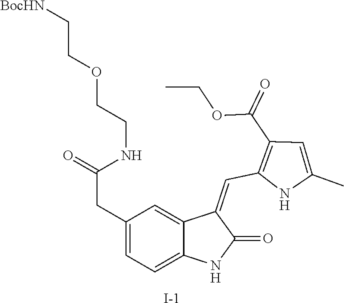 |
| 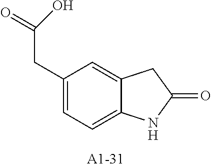 | 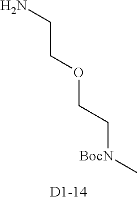 | 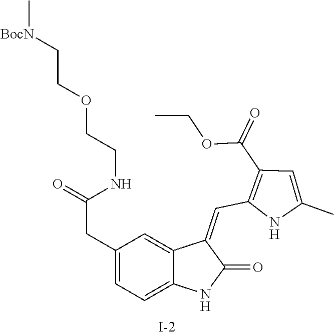 |
| 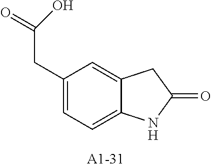 | 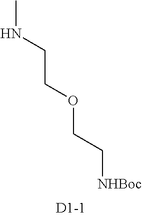 | 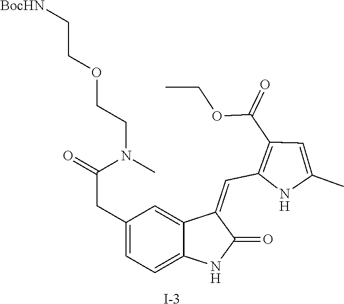 |
| 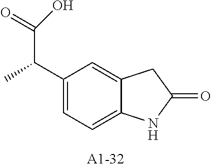 | 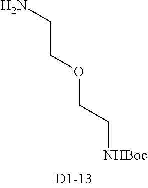 | 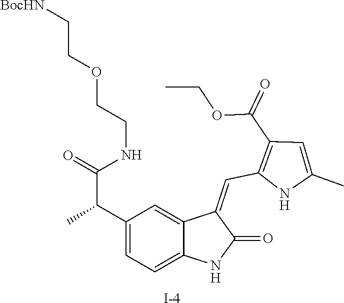 |
| 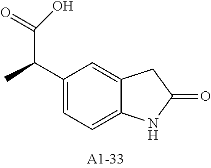 | 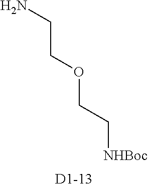 | 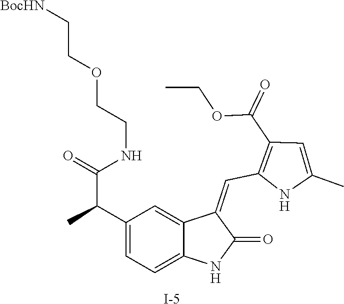 |
| 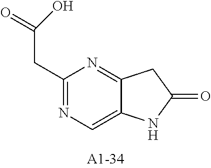 | 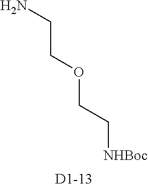 | 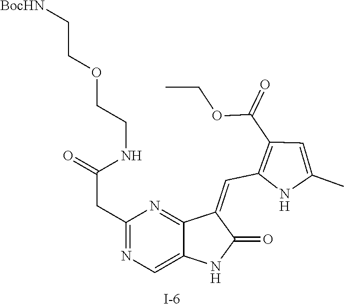 |
| 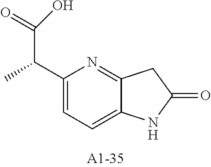 | 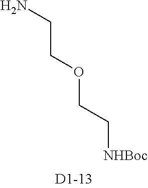 | 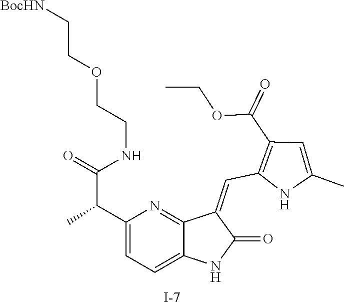 |
| 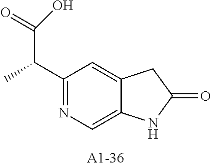 | 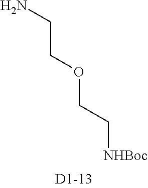 | 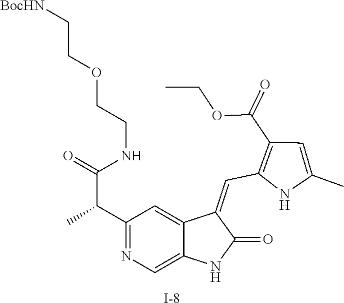 |
| 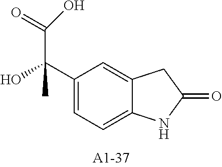 | 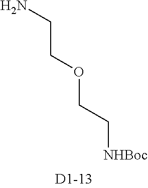 | 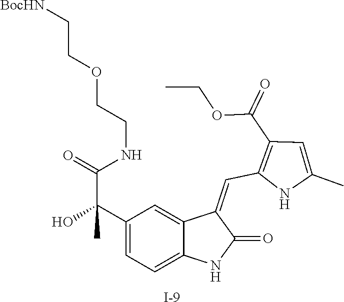 |

General Method J

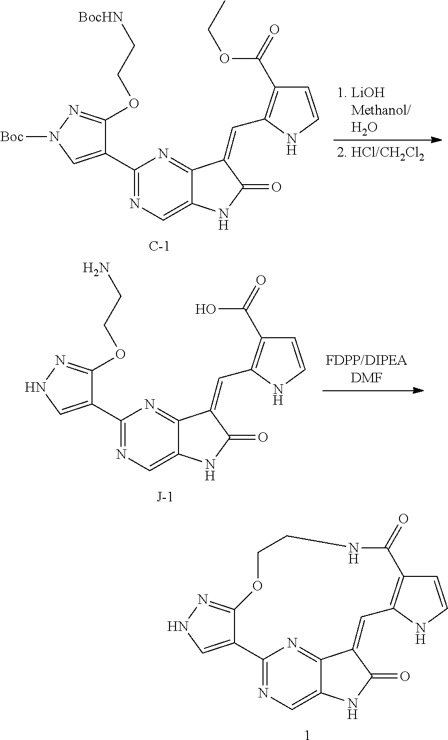

Step 1. To a solution of C-1 (1.0 eq.) in MeOH (0.2 M) is added LiOH (3 eq) in H₂O (1 M). The mixture is stirred at 60° C. until the hydrolysis reaction is completed. The solution is cooled to ambient temperature, concentrated to remove methanol, acidified by aqueous HCl (1 N) until pH ˜4-5, and then extracted with CH₂Cl₂. The combined extracts are dried over Na₂SO₄, concentrated, and dried under vacuum. The resulting crude solid is dissolved in CH₂Cl₂ (0.2 M) and to the solution is added a solution of HCl in dioxane (4 eq HCl). The solution is stirred at 40° C. until the de-Boc is completed. The solvents are removed under rotavap and the residue is dried under vacuum to provide a crude J-1 which is used for the next step without purification.

Step 2. To a solution of J-1 (1 eq.) in DMF (0.2 M) are added DIPEA (3 eq.) and pentafluorophenyl diphenylphosphinate (FDPP) (1.1 eq). The solution is stirred at ambient temperature until the amide formation is completed. The mixture is diluted with water and extracted with EtOAc for three times. The combined extracts are washed with water for three times, aqueous HCl (1N), saturated aqueous Na₂CO₃ and brine, dried over Na₂SO₄, and concentrated. The resulting residue is purified by a silica gel column to afford compound 1.

Following General Procedure J, Compounds 1-66 are prepared from corresponding C-1-C-66, Compounds 67-82 from D-1-D-16, Compounds 83-98 from E-1-E-16, Compound 99 from F-1, Compounds 100-103 from G-1-G-4, Compounds 104-113 from H-1-H-10, and Compounds 114-122 from I-1-I-9.

General Method K

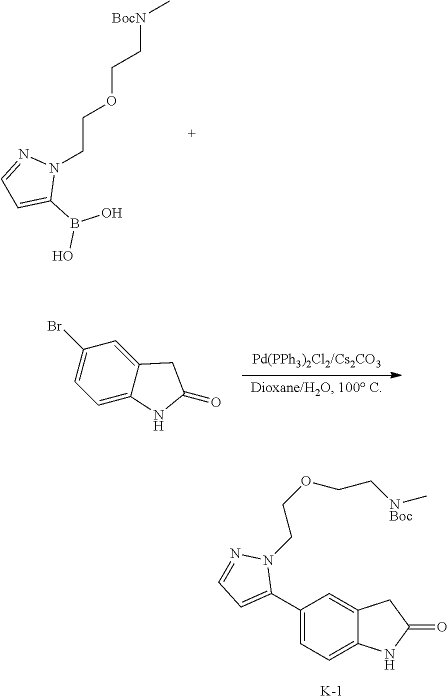

To a mixture of [2-[2-[2-[tert-butoxycarbonyl(methyl)amino]ethoxy]ethyl]pyrazol-3-yl]boronic acid (1 eq), 5-bromoindolin-2-one (1.3 eq), and Cs₂CO₃ (3 eq) in dioxane and H₂O is added Pd(PPh₃)₂Cl₂ (0.1 eq) under nitrogen. The mixture is stirred at 100° C. for 16 h under N₂, then cooled and concentrated in vacuum. The residue is purified by column chromatography (SiO₂) to give tert-butyl N-methyl-N-[2-[2-[5-(2-oxoindolin-5-yl) pyrazol-1-yl]ethoxy]ethyl]carbamate (K-1)

General Method L

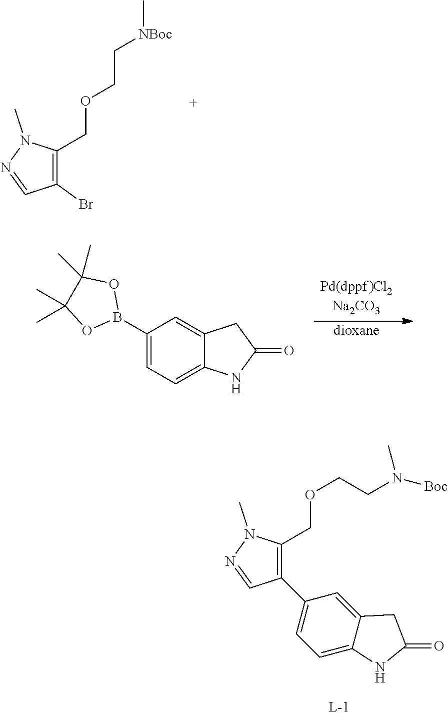

To a solution of tert-butyl N-[2-[(4-bromo-2-methyl-pyrazol-3-yl)methoxy]ethyl]-N-methyl-carbamate (1 eq), 5-(4,4,5,5-tetramethyl-1,3,2-dioxaborolan-2-yl)indolin-2-one (1.5 eq) in dioxane (17 mL) is added Pd(dppf)Cl₂ (0.1 eq) and aqueous Na₂CO₃ (2 M, 3.0 eq) under nitrogen. The mixture is stirred at 100° C. for 2 h under nitrogen atmosphere. On completion, the mixture is concentrated under vacuum to afford the title crude compound. The residue is purified by silica gel column to afford tert-butyl N-methyl-N-[2-[[2-methyl-4-(2-oxoindolin-5-yl) pyrazol-3-yl]methoxy]ethyl]carbamate (L-1).

General Method M

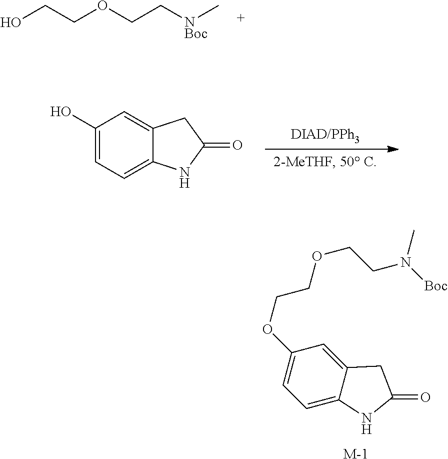

To a solution of 5-hydroxyindolin-2-one (1 eq), PPh₃ (2.2 eq) and tert-butyl N-[2-(2-hydroxyethoxy)ethyl]-N-methyl-carbamate (2.0 eq) in 2-MeTHF is DIAD (2.2 eq) in an ice-bath. The mixture is stirred at 50° C. for 16 h, quenched with MeOH, and concentrated under vacuum. The residue is purified by silica gel column to afford tert-butyl N-methyl-N-[2-[2-(2-oxoindolin-5-yl)oxyethoxy]ethyl]carbamate (M-1).

General Method N

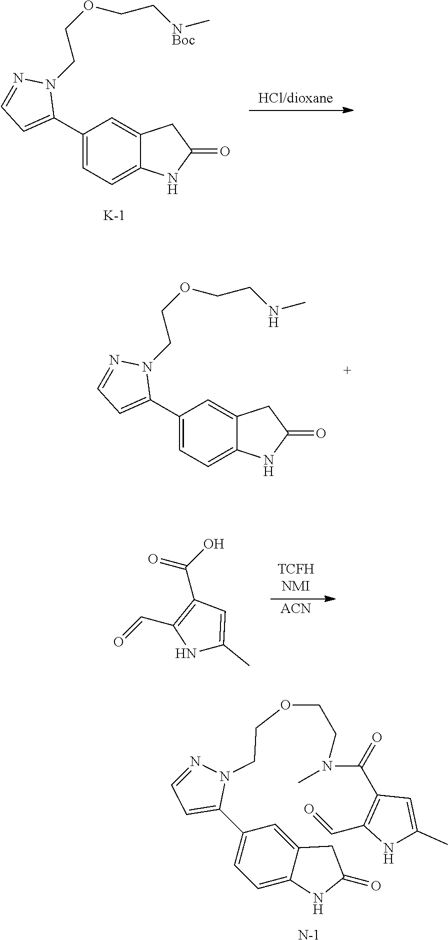

Step 1. To a solution of tert-butyl N-methyl-N-[2-[2-[5-(2-oxoindolin-5-yl)pyrazol-1-yl]ethoxy]ethyl]carbamate (1 eq) in DCM is added HCl/dioxane (4 M, 10 eq) and the resulting mixture is stirred at 25° C. for 1 h. The reaction mixture is concentrated under vacuum to give 5-[2-[2-[2-(methylamino) ethoxy]ethyl]pyrazol-3-yl]indolin-2-one HCl salt.

Step 2. To a solution of 5-[2-[2-[2-(methylamino)ethoxy]ethyl]pyrazol-3-yl]indolin-2-one HCl salt (0.34 mmol), 2-formyl-5-methyl-1H-pyrrole-3-carboxylic acid (1 eq) in acetonitrile is added 1-methylimidazole (3 eq) and [chloro(dimethylamino)methylene]-dimethyl-ammonium hexafluorophosphate (1.5 eq) and the mixture is stirred at 25° C. for 0.5 h. The reaction mixture is concentrated under vacuum and purified by column chromatography on silica gel. The crude product is triturated with MeOH at 25° C. for 10 min and then filtered to give 2-formyl-N,5-dimethyl-N-[2-[2-[5-(2-oxoindolin-5-yl)pyrazol-1-yl]ethoxy]ethyl]-1H-pyrrole-3-carboxamide (N-1).

General Method O

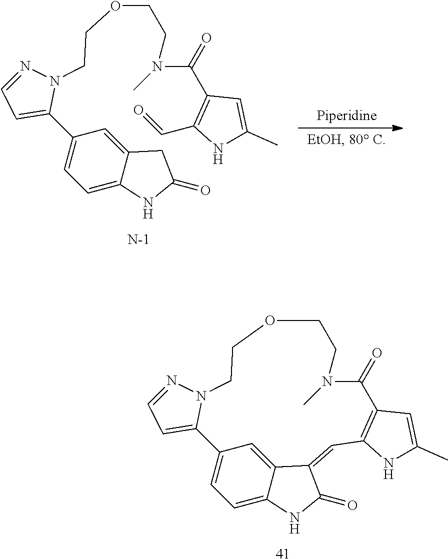

To a solution of N-1 (1 eq) in EtOH is added piperidine (2 eq). The mixture is stirred at 80° C. for 1 h. The reaction mixture is cooled and concentrated under vacuum. The crude product is triturated with MeOH at 25° C. for 10 min to provide the tritle compound (41).

### Example 1

### Preparation of methyl 2-[(Z)-(5-chloro-2-oxo-1H-pyrrolo[2,3-c]pyridin-3-ylidene)methyl]-1H-pyrrole-3-carboxylate (A-27) According to General Method A

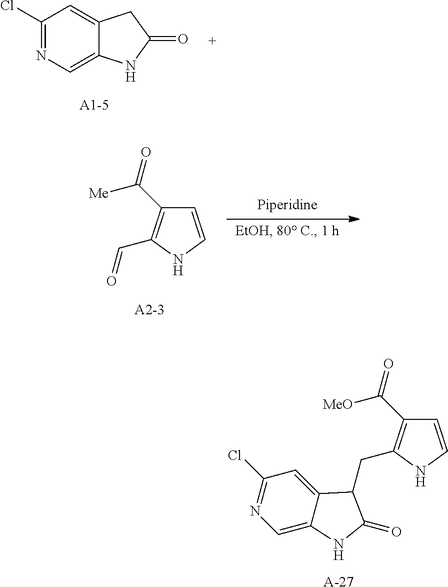

The mixture of 5-chloro-1,3-dihydropyrrolo[2,3-c]pyridin-2-one (1.0 g, 5.93 mmol, 1 eq), methyl 2-formyl-1H-pyrrole-3-carboxylate (908 mg, 5.93 mmol, 1 eq) and piperidine (1.01 g, 11.86 mmol, 1.17 mL, 2.0 eq) in EtOH (100 mL) was stirred at 80° C. for 1 h. On completion, the mixture was cooled to ambient temperature and the product was precipitated out. The solid was filtered, washed with EtOH (30 mL), and dried in vacuo to afford methyl 2-[(Z)-(5-chloro-2-oxo-1H-pyrrolo[2,3-c]pyridin-3-ylidene)methyl]-1H-pyrrole-3-carboxylate (1.6 g, 4.21 mmol, 71% yield) as yellow powder. ¹H NMR (400 MHz, DMSO-d₆) δ (ppm).

A-28-A31 were prepared following a similar procedure as A-27.

| | | ¹NMR (400 MHz, DMSO-d₆) δ |
|---|---|---|
| Comp. # | Structure | (ppm) |
| A-27 | 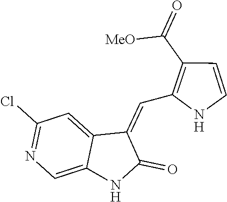 [SMILES: COC(=O)c1cc[nH]c1/C=C1\C(=O)Nc2cnc(Cl)cc21] | 14.01 (s, 1H), 11.50 (s, 1H), 8.58 (s, 1H), 8.01 (s, 1H), 7.78 (s, 1H), 7.61-7.65 (m, 1H), 6.87 (m, 1H), 3.87 (s, 3H) |
| A-28 | 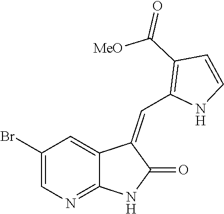 [SMILES: COC(=O)c1cc[nH]c1/C=C1\C(=O)Nc2ncc(Br)cc21] | 13.71 (s, 1H), 11.88 (s, 1H), 8.40 (s, 1H), 8.22-8.23 (d, J = 2.0 Hz, 1H), 8.10 (d, J = 2.0 Hz, 1H), 7.44 (m, 1H), 6.80-6.81 (m, 1H), 3.84 (s, 3H). |
| A-29 | 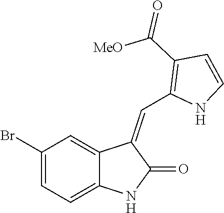 [SMILES: COC(=O)c1cc[nH]c1/C=C1\C(=O)Nc2ccc(Br)cc21] | 13.84 (s, 1H), 11.27 (s, 1H), 8.38 (s, 1H), 7.63-7.64 (d, J = 2.0 Hz, 1H), 7.38-7.39 (m, 2H), 6.88-6.89 (m, 1H), 6.77 (m, 1H), 3.84 (s, 3H) |
| A-30 | 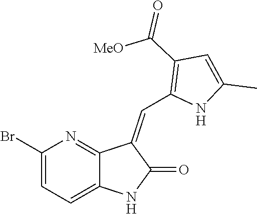 [SMILES: COC(=O)c1cc(C)[nH]c1/C=C1\C(=O)Nc2ccc(Br)nc21] | 14.10 (s, 1H), 8.33 (s, 1H), 7.54 (d, J = 8.4 Hz, 1H), 7.31 (d, J = 8.4 Hz, 1H), 6.68 (d, J = 2.4 Hz, 1H), 3.83 (s, 3H), 2.47 (s, 3H) |
| A-31 | 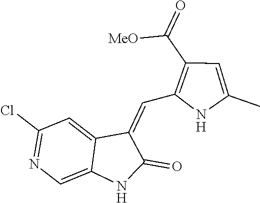 [SMILES: COC(=O)c1cc(C)[nH]c1/C=C1\C(=O)Nc2cnc(Cl)cc21] | 13.96 (s, 1H), 11.35 (s, 1H), 8.47 (s, 1H), 7.99 (s, 1H), 7.61 (s, 1H), 6.66 (s, 1H), 3.85 (s, 3H), 2.40 (s, 3H) |

### Example 2

### Preparation of tert-butyl 3-[113-(tert-butoxycarbonylamino)propoxy]-4-(4,4,5,5-tetramethyl-1,3,2-dioxaborolan-2-yl)pyrazole-1-carboxylate (B-I-2) according to General Method B-I

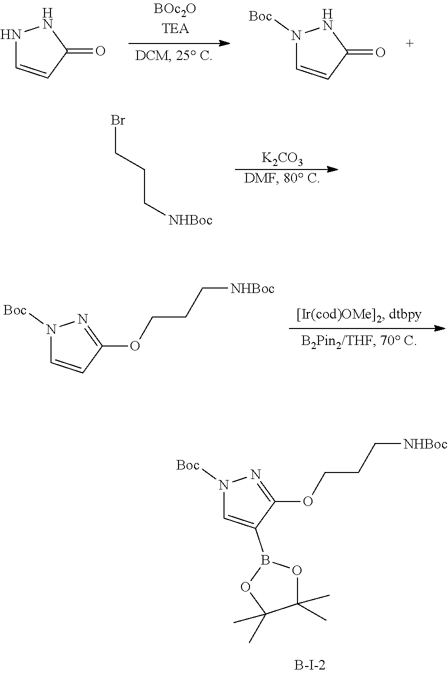

Step 1: To a solution of 1,2-dihydropyrazol-3-one (5.0 g, 59.5 mmol, 1 eq) and TEA (7.82 g, 77.3 mmol, 10.7 mL, 1.3 eq) in DCM (200 mL) was added (Boc)₂O (14.28 g, 65.4 mmol, 15.0 mL, 1.1 eq) at 25° C. The mixture was stirred at 25° C. for 4 h. On completion, the mixture was diluted with DCM (200 mL), washed with brine (100 mL). The organic layer was dried over Na₂SO₄, filtered and concentrated in vacuum to afford tert-butyl 5-oxo-1H-pyrazole-2-carboxylate (9.0 g, 47.4 mmol, 79.7% yield, 97% purity) as light yellow powder. ¹H NMR (400 MHz, CDCl₃) δ (ppm) 7.81 (d, J=3.2 Hz, 1H), 5.90 (d, J=3.2 Hz, 1H), 1.63 (s, 9H).

Step 2. To a solution of tert-butyl 5-oxo-1H-pyrazole-2-carboxylate (7.0 g, 38.0 mmol, 1 eq) and tert-butyl N-(3-bromopropyl)carbamate (9.95 g, 41.80 mmol, 1.1 eq) in DMF (21 mL) was added K₂CO₃ (7.88 g, 57.0 mmol, 1.5 eq). The mixture was stirred at 80° C. for 16 h. On completion, the mixture was diluted with EtOAc (100 mL), washed with brine (2×40 mL). The organic layer was dried over Na₂SO₄, filtered and concentrated in vacuum. The crude residue was purified by combi-flash (40 g silica column, 0-40 EtOAc in PE, eluted˜10%) to give tert-butyl 3-[3-(tert-butoxycarbonylamino) propoxy]pyrazole-1-carboxylate (8.2 g, 22.8 mmol, 60% yield, 95% purity) as white oil. ¹H NMR (400 MHz, DMSO-d₆) δ (ppm) 8.08 (d, J=3.2 Hz, 1H), 6.93-6.83 (m, 1H), 6.08 (d, J=3.2 Hz, 1H), 4.16 (t, J=6.3 Hz, 2H), 3.10-2.99 (m, 2H), 2.53-2.50 (m, 2H), 1.55 (s, 9H), 1.37 (s, 9H).

Step 3. To a solution of tert-butyl 3-[3-(tert-butoxycarbonylamino)propoxy]pyrazole-1-carboxylate(1.50 g, 4.39 mmol, 1 eq) and Pin₂B₂(2.23 g, 8.7 mmol, 2.0 eq) in THF (30 mL) was added (1,5-Cyclooctadiene)(methoxy)iridium(I) dimer (291.2 mg, 439 umol, 0.1 eq) and 4-tert-butyl-2-(4-tert-butyl-2-pyridyl)pyridine (235 mg, 878 umol, 0.2 eq) under nitrogen atmosphere. The mixture was stirred at 70° C. for 16 h. On completion, the mixture was diluted with EtOAc (50 mL), washed with brine (2×20 mL). The organic layer was dried over Na₂SO₄, concentrated in vacuum, and purified by silica gel column (40 g, 0-100% EA in PE, eluted ˜35%) to afford tert-butyl 3-[3-(tert-butoxycarbonylamino)propoxy]-4-(4,4,5,5-tetramethyl-1,3,2-dioxaborolan-2-yl)pyrazole-1-carboxylate (B-I-2, 2.3 g, 2.9 mmol, 67.2% yield) as white oil. ¹H NMR (400 MHz, DMSO-d₆) δ (ppm) 8.10 (s, 1H), 6.81 (t, J=5.2 Hz, 1H), 3.80-3.76 (m, 2H), 3.06 (q, J=6.4 Hz, 2H), 1.84-1.82 (m, 1H), 1.55 (s, 9H), 1.37 (s, 9H), 1.25 (s, 12H).

### Example 3

### Preparation of tert-butyl N-[3-[1-methyl-4-(4,4,5,5-tetramethyl-1,3,2-dioxaborolan-2-yl)pyrazol-3-yl]oxypropyl]carbamate (B-I-5) according to General Method B-I

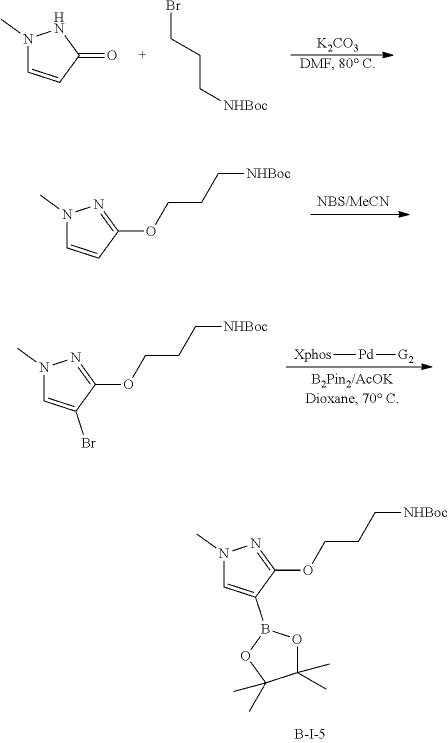

Step 1. To a solution of 2-methyl-1H-pyrazol-5-one (7 g, 71.35 mmol, 1 eq) and tert-butyl N-(3-bromopropyl)carbamate (22.09 g, 92.76 mmol, 1.3 eq) in DMF (70 mL) was added K₂CO₃ (14.79 g, 107.03 mmol, 1.5 eq). The mixture was stirred at 80° C. for 16 hours. On completion, the mixture was cooled to 25° C., diluted with water (100 mL), extracted with EA (3*60 mL). The combined organic layers were washed with brine (20 mL), dried over Na₂SO₄, filtered and concentrated in vacuum. The residue was purified by column chromatography on silica gel (PE: EA=30: 1-3:1) to give tert-butyl N-[3-(1-methylpyrazol-3-yl)oxypropyl]carbamate (15 g, 58.75 mmol, 82.3% yield) as colorless gum. ¹H NMR (400 MHz, CDCl₃) δ (ppm) 7.10 (d, J=2.4 Hz, 1H), 5.58 (d, J=2.4 Hz, 1H), 4.95-4.92 (m, 1H), 4.18-4.15 (m, 2H), 3.71 (s, 3H), 3.30-3.25 (m, 2H), 1.94-1.90 (m, 2H), 1.43 (s, 9H).

Step 2. To a solution of tert-butyl N-[3-(1-methylpyrazol-3-yl)oxypropyl]carbamate (7 g, 27.42 mmol, 1 eq) in ACN (40 mL) was added NBS (5.03 g, 28.24 mmol, 1.03 eq) at 25° C. The mixture was stirred at 25° C. for 16 hours. On completion, the reaction mixture was concentrated under reduced pressure. The residue was purified by column chromatography on silica gel (PE: EA=25:1-2:1) to give tert-butyl N-[3-(4-bromo-1-methyl-pyrazol-3-yl)oxypropyl]carbamate (7.3 g, 21.84 mmol, 79.6% yield) as colorless oil. ¹H NMR (400 MHz, CDCl₃) δ (ppm) 7.18 (s, 1H), 5.04-5.02 (m, 1H), 4.28-4.25 (m, 2H), 3.72 (s, 3H), 3.32-3.27 (m, 2H), 1.97-1.94 (m, 2H), 1.44 (s, 9H).

Step 3. To the mixture of tert-butyl N-[3-(4-bromo-1-methyl-pyrazol-3-yl)oxypropyl]carbamate (3.0 g, 8.98 mmol, 1 eq), AcOK (2.64 g, 26.93 mmol, 3.0 eq) and Pin₂B₂(10.26 g, 40.39 mmol, 4.5 eq) in dioxane (50 mL) was added Xphos-Pd-G2 (706 mg, 897 umol, 0.1 eq) under nitrogen. The mixture was stirred at 60° C. for 16 h under nitrogen atmosphere. On completion, the mixture was cooled to ambient temperature, diluted with PE (200 mL) and filtered. The organic layer was concentrated in vacuum to afford grass-green oil. The crude was purified by silica gel column (20 g, 0-100% EtOAc in PE, 15 min, eluted ˜60%) to afford tert-butyl N-[3-[1-methyl-4-(4,4,5,5-tetramethyl-1,3,2-dioxaborolan-2-yl)pyrazol-3-yl]oxypropyl]carbamate (B-I-5, 2.9 g, 6.08 mmol, 68% yield) as brown gum. LCMS: m/z 381.9 (M+1)⁺

### Example 4

### Preparation of tert-butyl N-[3-[2-(4,4,5,5-tetramethyl-1,3,2-dioxaborolan-2-yl)phenoxy]propyl]carbamate (B-II-1) according to General Method B-II

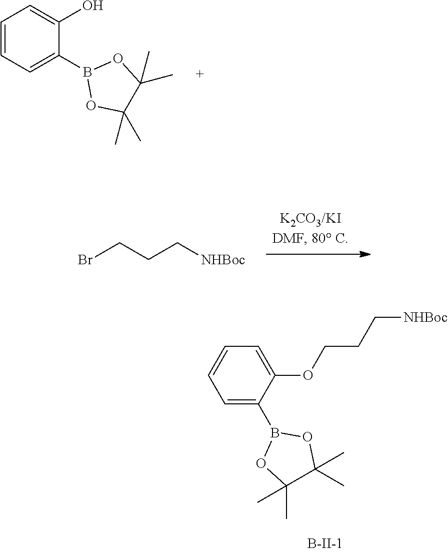 [SMILES: CC1(C)OB(c2ccccc2O)OC1(C)C ; CC(C)(C)OC(=O)NCCCBr ; CC(C)(C)OC(=O)NCCCOc1ccccc1B1OC(C)(C)C(C)(C)O1]

To the mixture of 2-(4,4,5,5-tetramethyl-1,3,2-dioxaborolan-2-yl)phenol (10 g, 45.4 mmol, 1 eq), K₂CO₃ (18.8 g, 136 mmol, 3.0 eq) and KI (754 mg, 4.54 mmol, 0.1 eq) in DMF (50 mL) was added tert-butyl N-(3-bromopropyl)carbamate (11.9 g, 50.0 mmol, 1.1 eq). The mixture was stirred at 80° C. for 16 h. On completion, the mixture was cooled, diluted with ethyl acetate (200 mL), washed with brine (2×50 mL), dried over Na₂SO₄, filtered and concentrated in vacuo. The residue was purified by column chromatography (SiO₂, 0-100% EtOAc in PE, eluted ˜25%) to give tert-butyl N-[3-[2-(4,4,5,5-tetramethyl-1,3,2-dioxaborolan-2-yl)phenoxy]propyl]carbamate (B-II-1, 10 g, 22.5 mmol, 49.5% yield, 85% purity) as colorless gum. ¹H NMR (400 MHz, CDCl₃) δ (ppm) 7.70-7.76 (m, 1H), 7.37-7.44 (m, 1H), 6.95-7.01 (m, 1H), 6.86-6.92 (m, 1H), 5.46-5.62 (m, 1H), 4.05-4.12 (m, 2H), 3.36-3.49 (m, 2H), 1.96-2.05 (m, 2H), 1.44 (s, 10H), 1.37 (s, 12H).

### Example 5

### Preparation of tert-butyl N-[3-[2-(4,4,5,5-tetramethyl-1,3,2-dioxaborolan-2-yl)phenoxy]ethyl]carbamate (B-II-2) according to General Method B-II

B-II-2 was prepared using a similar procedure as B-II-1.

### Example 6

### Preparation of [2-[2-[2-(tertbutoxycarbonylamino) ethoxy]ethyl]pyrazol-3-yl]boronic acid (B-III-7) according to General Method B-III

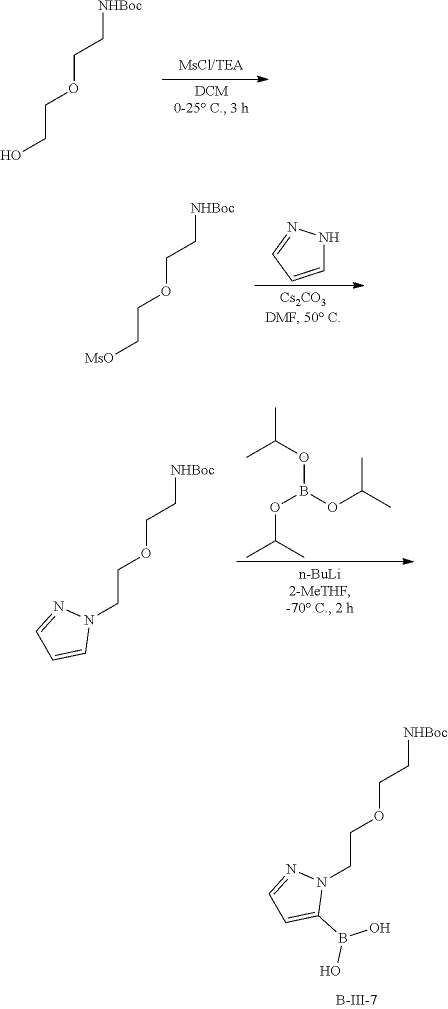 [SMILES: CC(C)(C)OC(=O)NCCOCCO ; CC(C)(C)OC(=O)NCCOCCOS(C)(=O)=O ; c1cn[nH]c1 ; CC(C)(C)OC(=O)NCCOCCn1cccn1 ; CC(C)OB(OC(C)C)OC(C)C ; CC(C)(C)OC(=O)NCCOCCn1nccc1B(O)O]

The mixture of tert-butyl N-[2-(2-hydroxyethoxy)ethyl]carbamate (20.0 g, 97.4 mmol, 1 eq) and TEA (29.6 g, 292 mmol, 40.7 mL, 3.0 eq) in DCM (500 mL) in an ice bath was added MsCl (16.7 g, 146 mmol, 11.3 mL, 1.5 eq). The mixture was stirred at 25° C. for 2 h. On completion. The mixture was quenched with water (300 mL), combined organic layer was washed with sat. NaHCO₃(80 mL), brine (300 mL), dried over sodium sulfate, concentrated in vacuum to afford 2-[2-(tert-butoxycarbonylamino) ethoxy]ethyl methanesulfonate (25.0 g, 75.0 mmol, 76.9% yield) as light yellow gum. ¹H NMR (400 MHz, DMSO-d₆) δ=6.79 (s, 1H), 4.30 (t, J=4.8 Hz, 2H), 3.64 (t, J=4.8 Hz, 2H), 3.42 (t, J=6.0 Hz, 2H), 3.18 (s, 3H), 3.09 (t, J=6.0 Hz, 2H), 1.38 (s, 9H).

Step 2. To a solution of 2-[2-(tert-butoxycarbonylamino)ethoxy]ethyl methanesulfonate (16.0 g, 56.5 mmol, 1 eq) in DMF (80 mL) was added 1H-pyrazole (3.84 g, 56.5 mmol, 1.0 eq) and Cs₂CO₃ (36.8 g, 112 mmol, 2 eq). The mixture was stirred at 50° C. for 2 h. On completion, the mixture was quenched with water (200 mL), diluted with EA (3*100 mL). Combined organic layer was washed with brine (200 mL), dried over Na₂SO₄, concentrated in vacuum to afford crude. The residue was purified by column chromatography (SiO₂, DCM: MeOH=20:1) to afford tert-butyl N-[2-(2-pyrazol-1-ylethoxy)ethyl]carbamate (13.0 g, 48.3 mmol, 85.6% yield) was a colorless oil. LCMS: m/z 256.0 (M+1)⁺.

Step 3. To a solution of tert-butyl N-[2-(2-pyrazol-1-ylethoxy)ethyl]carbamate (2.00 g, 7.83 mmol, 1 eq) in 2-MeTHF (150 mL) at −70° C. was added n-BuLi (2.5 M, 9.40 mL, 3 eq) dropwise. The mixture was stirred at 25° C. for 0.5 h followed by addition of triisopropylborate (2.21 g, 11.7 mmol, 2.70 mL, 1.5 eq) in 2-MeTHF (150 mL) at −70° C. The mixture was stirred at 25° C. for 1.5 h. On completion, the mixture was quenched with MeOH (50 mL), concentrated in vacuum and purified by reversed-phase HPLC to afford [2-[2-[2-(tertbutoxycarbonylamino) ethoxy]ethyl]pyrazol-3-yl]boronic acid (B-III-7, 500 mg, 18.1% yield) as white powder. ¹H NMR (400 MHz, DMSO-d₆) δ (ppm) 8.36 (m, 2H), 7.39 (s, 1H), 6.71 (s, 2H), 4.50 (t, J=4.8 Hz, 2H), 3.68 (t, J=4.8 Hz, 2H), 3.33 (t, J=6.0 Hz, 2H), 3.01 (t, J=6.0 Hz, 2H), 1.37 (s, 9H). LCMS: m/z 300 (M+1)⁺.

### Example 7

### Preparation of [2-[2-[2-[tert-butoxycarbonyl(methyl)amino]ethoxy]ethyl]pyrazol-3-yl]boronic acid (B-III-8) according to General Method B-III

B-II-8 was prepared using similar procures as B-III-7. LCMS: m/z 314.1 (M+1)⁺.

### Preparation of [2-[2-[benzyloxycarbonyl-[2-(tert-butoxycarbonylamino)ethyl]amino]ethyl]pyrazol-3-yl]boronic acid (B-III-9)

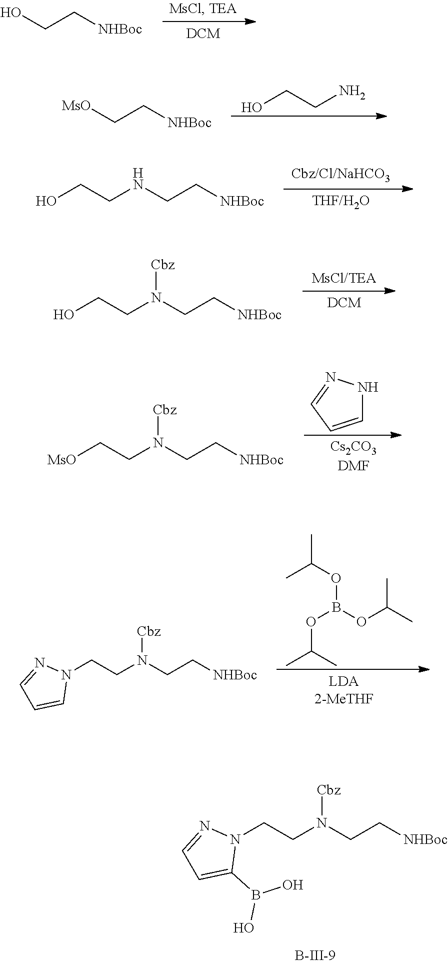

Step 1. To a mixture of tert-butyl N-(2-hydroxyethyl)carbamate (1.00 g, 6.20 mmol, 1 eq.), and TEA (941 mg, 9.31 mmol, 1.5 eq.) in DCM (30 mL) was added MsCl (852 mg, 7.44 mmol, 1.2 eq.) in an ice-bath. The mixture was stirred at 25° C. for 3 hours. On completion, the mixture was quenched with water (10 mL) and diluted with DCM (20 mL). The organic layer was washed with sat. NaHCO₃ (50 mL), brine (50 mL), dried over sodium sulfate, and concentrated in vacuum to afford (2-(tert-butoxycarbonylamino)ethyl methanesulfonate (1.20 g, 4.51 mmol, 72% yield, 90% purity) as light yellow oil. ¹H NMR (400 MHz, CDCl₃) δ=4.90 (s, 1H), 4.21 (t, J=5.2 Hz, 2H), 3.41 (dd, J=10.8, 5.6 Hz, 2H), 2.97 (s, 3H), 1.38 (s, 9H).

Step 2. 2-(tert-butoxycarbonylamino)ethyl methanesulfonate (9.00 g, 37.0 mmol, 1.0 eq.) and 2-aminoethanol (22.9 g, 376 mmol, 10 eq.) was heated to 80° C. for 16 h. The mixture was quenched with water (200 mL), diluted with EtOAc (3×100 mL). Combined organic layer was washed with brine (100 mL), dried over sodium sulfate, concentrated in vacuum to afford (tert-butyl N-[2-(2-hydroxyethylamino)ethyl]carbamate (10.0 g, 36.7 mmol, 97.6% yield) as light yellow solid. ¹H NMR (400 MHz, DMSO-d₆) δ=6.77-6.65 (m, 1H), 4.52-4.34 (m, 1H), 3.42 (t, J=5.7 Hz, 2H), 3.04-2.93 (m, 2H), 2.57-2.52 (m, 4H), 2.52-2.50 (m, 2H), 1.38 (s, 9H).

Step 3. To a solution of tert-butyl N-[2-(2-hydroxyethylamino)ethyl]carbamate (3.00 g, 14.6 mmol, 1 eq.) in THF (50 mL) and H₂O (12 mL) was added NaHCO₃ (3.70 g, 44.0 mmol, 3 eq.) and CbzCl (3.26 g, 19.0 mmol, 1.3 eq.). The mixture was stirred at 20° C. for 16 hr. On completion, the mixture was quenched with water (150 mL), extracted with EtOAc (3×100 mL). Combined organic layers were washed with brine (150 mL), dried over sodium sulfate, concentrated in vacuum, and the residue was purified by flash chromatography (40 g silica gel column, EtOAc in PE from 0% to 100%) to afford benzyl N-[2-(tert-butoxycarbonylamino)ethyl]-N-(2-hydroxyethyl) Carbamate (3.40 g, 9.54 mmol, 64.9% yield) as colorless gum. ¹H NMR (400 MHz, DMSO-d₆) δ=7.44-7.27 (m, 5H), 6.94-6.80 (m, 1H), 5.07 (s, 2H), 4.78-4.68 (m, 1H), 3.48 (d, J=3.5 Hz, 2H), 3.31-3.24 (m, 4H), 3.07 (d, J=6.3 Hz, 2H), 1.37 (s, 9H); LCMS: m/z 239.1 (M+1-100)⁺.

Step 4. To a solution of benzyl N-[2-(tert-butoxycarbonylamino)ethyl]-N-(2-hydroxyethyl)carbamate (3.40 g, 10.0 mmol, 1 eq.) and TEA (3.05 g, 30.1 mmol, 3.0 eq.) in DCM (100 mL) was added MsCl (1.73 g, 15.0 mmol, 1.17 mL, 1.5 eq.) in an ice-bath. The mixture was stirred at 25° C. for 3 hours. On completion, the mixture was quenched with water (150 mL), diluted with DCM (3×150 mL). Combined organic layer was washed with Sat. NaHCO₃ (100 mL), brine (80 mL), dried over sodium sulfate, concentrated in vacuum to afford crude (2-[benzyloxycarbonyl-[2-(tert-butoxycarbonylamino)ethyl]amino]ethyl methanesulfonate, 4.00 g, 9.60 mmol, 95% yield)), which was obtained as a light yellow gum. LCMS: m/z 317.1 (M+1−100)⁺.

Step 5. To a solution of 2-[benzyloxycarbonyl-[2-(tert-butoxycarbonylamino)ethyl]amino]ethyl methanesulfonate (5.30 g, 12.7 mmol, 1.2 eq.) in DMF (40 mL) was added 1H-pyrazole (721 mg, 10.6 mmol, 1 eq.) and Cs₂CO₃ (6.91 g, 21.2 mmol, 2 eq.). The mixture was stirred at 50° C. for 3 hours. On completion, the mixture was quenched with water (50 mL), and extracted with EtOAc (3×50 mL). Combined organic layers were washed with brine (50 mL), dried over sodium sulfate, concentrated in vacuum. The residue was purified by flash chromatography (12 g silica gel column, EtOAc in PE from 0% to 100%) to obtain benzyl N-[2-(tert-butoxycarbonylamino)ethyl]-N-(2-pyrazol-1-ylethyl)carbamate (3.60 g, 7.88 mmol, 74.2% yield) as a light yellow gum. LCMS: m/z 389.4 (M+1)⁺.

Step 6. To a mixture of benzyl N-[2-(tert-butoxycarbonylamino)ethyl]-N-(2-pyrazol-1-ylethyl) carbamate (1.60 g, 4.12 mmol, 1 eq.) in 2-MeTHF (70 mL) was dropped LDA (2 M, 6.18 mL, 3 eq.) at −70° C. under N₂ atmosphere. The mixture was stirred at −70° C. for 0.5 hours, and then triisopropyl borate (1.55 g, 8.24 mmol, 2 eq.) was added. The result mixture was stirred at −70° C. for 1.5 h under N₂ atmosphere. On completion, the mixture was quenched with MeOH (10 mL), and extracted with EtOAc (3×60 mL). Combined organic layers were washed with pure water (70 mL) and the water phase was lyophilized. The residue was purified by reverse phase preparative HPLC (0.5% FA as additives) to obtain [2-[2-[benzyloxycarbonyl-(tert-butoxycarbonylamino)ethyl]amino]ethyl]pyrazol-3-yl]boronic acid (B-III-9, 500 mg, 0.925 mmol, 22.4% yield) as a white solid. LCMS: m/z 433.4 (M+1)⁺.

### Example 8

### Preparation of tert-butyl N-[2-[(4-bromo-2-methyl-pyrazol-3-yl) methoxy]ethyl]carbamate (B-V-1) and tert-butyl N-[2-[[2-methyl-4-(4,4,5,5-tetramethyl-1,3,2-dioxaborolan-2-yl) pyrazol-3-yl]methoxy]ethyl]carbamate (B-VI-1)

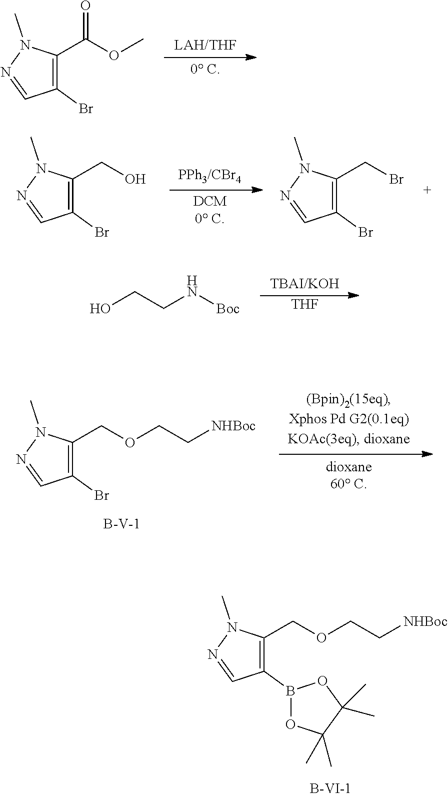

Step 1. To a solution of methyl 4-bromo-2-methyl-pyrazole-3-carboxylate (9.5 g, 43.4 mmol, 1 eq) in THF (100 mL) was added LiAlH₄ (1.65 g, 43.4 mmol, 1 eq). The mixture was stirred at 0° C. for 15 min and then slowly quenched with water (0.086 mL) followed by addition of saturated sodium hydroxide (1.65 mL) and water (4.8 mL). The reaction mixture was filtered and concentrated under reduced pressure to give (4-bromo-2-methyl-pyrazol-3-yl) methanol (7.75 g, 40.6 mmol, 93.5% yield) as a colorless oil. LCMS: 190.9 (M+1)⁺.

Step 2. To a solution of (4-bromo-2-methyl-pyrazol-3-yl) methanol (7.75 g, 40.6 mmol, 1 eq) in DCM (70 mL) was added CBr₄ (16.2 g, 48.7 mmol, 1.2 eq) followed by addition of a solution of PPh₃ (12.8 g, 48.7 mmol, 1.2 eq) in DCM (2 mL) dropwise at 0° C. The mixture was stirred at 0° C. for 0.5 h. The mixture was slowly quenched with water and extracted with EtOAc (3*100 mL). The combined organic layers were washed with brine (2*100 mL), dried over Na₂SO₄, filtered and concentrated in vacuum. The residue was purified by column chromatography on silica gel (PE: EA=25: 1-3:1) to give 4-bromo-5-(bromomethyl)-1-methyl-pyrazole (7.60 g, 29.9 mmol, 73.8% yield) as a colorless oil. ¹H NMR (400 MHz, DMSO-d₆) δ (ppm) 7.56 (s, 1H), 4.75 (s, 2H), 3.87 (s, 3H).

Step 3. To a solution of 4-bromo-5-(bromomethyl)-1-methyl-pyrazole (1.00 g, 3.94 mmol, 1 eq) in THF (2 mL) were added tert-butyl N-(2-hydroxyethyl)carbamate (952 mg, 5.91 mmol, 0.915 mL, 1.5 eq), tetrabutylammonium iodide (145 mg, 0.394 mmol, 0.1 eq) and KOH (663 mg, 11.8 mmol, 3 eq). The mixture was stirred at 25° C. for 16 hours under N₂. The reaction mixture was quenched with water (30 mL) and extracted with EtOAc (3*20 mL). The combined organic layers were washed with brine (2*10 mL), dried over Na₂SO₄, filtered and concentrated in vacuum. The residue was purified by column chromatography (SiO₂, Petroleum ether/Ethyl acetate=10/1 to 3/1) to give tert-butyl N-[2-[(4-bromo-2-methyl-pyrazol-3-yl) methoxy]ethyl]carbamate (B-V-1, 1.2 g, 3.12 mmol, 79.3% yield) as yellow oil. ¹H NMR (400 MHz, CDCl₃) δ=7.42 (s, 1H), 4.80 (s, 1H), 4.55 (s, 2H), 3.91 (s, 3H), 3.52-3.48 (m, 2H), 3.32 (d, J=5.2 Hz, 2H), 1.44 (s, 9H).

Step 4. To a solution of tert-butyl N-[2-[(4-bromo-2-methyl-pyrazol-3-yl)methoxy]ethyl]carbamate (1.00 g, 2.99 mmol, 1 eq), KOAc (880 mg, 8.98 mmol, 3 eq) and Pin₂B₂ (11.4 g, 44.9 mmol, 15 eq) in dioxane (10 mL) was added [2-(2-aminophenyl)phenyl]-chloro-palladium;dicyclohexyl-[3-(2,4,6-triisopropylphenyl)phenyl]phosphane (235 mg, 0.299 mmol, 0.1 eq) at 25° C. under nitrogen. The mixture was stirred at 60° C. for 12 hours under N₂. The reaction mixture was cooled and concentrated in vacuum. The residue was purified by column chromatography on silica gel (PE: EA=25:1-3:1) to give tert-butyl N-[2-[[2-methyl-4-(4,4,5,5-tetramethyl-1,3,2-dioxaborolan-2-yl) pyrazol-3-yl]methoxy]ethyl]carbamate (B-VI-1, 1.55 g, 2.64 mmol, 88.3% yield) as a yellow oil. ¹H NMR (400 MHz, CDCl₃) δ=7.70 (s, 1H), 4.73 (s, 2H), 3.90 (s, 3H), 3.50 (d, J=4.8 Hz, 2H), 3.32 (d, J=4.8 Hz, 2H), 1.44 (s, 9H), 1.31 (s, 12H).

### Example 9

### Preparation of tert-butyl N-[2-[(4-bromo-2-methyl-pyrazol-3-yl) methoxy]ethyl]-N-methyl-carbamate (B-V-2)

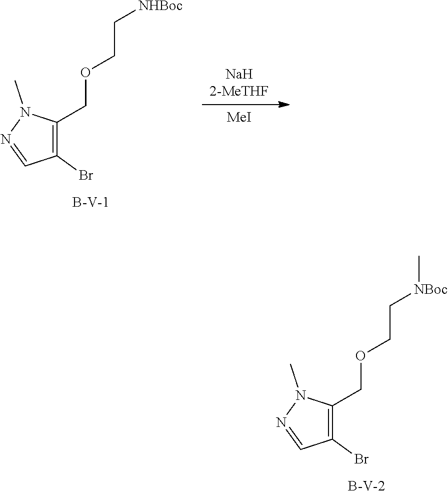 [SMILES: "MeI" ; Cn1ncc(Br)c1COCCNC(=O)OC(C)(C)C ; CN(CCOCc1c(Br)cnn1C)C(=O)OC(C)(C)C]

To a solution of tert-butyl N-[2-[(4-bromo-2-methyl-pyrazol-3-yl) methoxy]ethyl]carbamate (640 mg, 1.91 mmol, 1 eq) in 2-MeTHF (30 mL) was added NaH (191 mg, 4.79 mmol, 60%, 2.5 eq) at 0° C. The mixture was stirred at 0° C. for 0.5 h followed by addition of CH₃I (407.71 mg, 2.87 mmol, 1.5 eq). The mixture was stirred at ambient temperature for 1.5 h. On completion, the mixture was poured into the ice-water (40 mL), extracted with EtOAc (80 mL), washed with brine (50 mL). The organic layer was dried over sodium sulfate, filtered and concentrated in vacuum. The residue was purified by silica gel column (PE: EA=100: 0-100:35) to afford tert-butyl N-[2-[(4-bromo-2-methyl-pyrazol-3-yl) methoxy]ethyl]-N-methyl-carbamate (630 mg, 1.81 mmol, 94% yield) as colorless oil. LCMS: m/z 370.2 (M+Na)⁺.

### Example 10

### Preparation of tert-butyl N-[2-[benzyloxycarbonyl-[(4-bromo-2-methyl-pyrazol-3-yl)methyl]amino]ethyl]-N-methyl-carbamate (B-V-3)

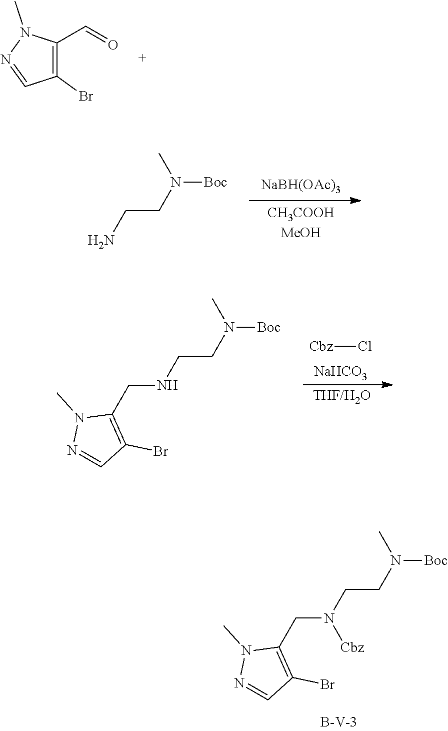

Step 1. To a solution of 4-bromo-2-methyl-pyrazole-3-carbaldehyde (4.55 g, 24.1 mmol, 1 eq) and tert-butyl N-(2-aminoethyl)-N-methyl-carbamate (8.39 g, 48.2 mmol, 8.61 mL, 2 eq) in MeOH (90 mL) was added AcOH (1.45 g, 24.1 mmol, 1.38 mL, 1 eq). The reaction was stirred at 25° C. for 0.5 h, cooled to 0° C., and treated with NaBH(OAc)₃ (7.66 g, 36.1 mmol, 1.5 eq). The mixture was stirred at 25° C. for 13 h, quenched with water (100 mL) and extracted with ethyl acetate (3*40 mL). The combined organic phase was dried over anhydrous sodium sulfate, filtered and concentrated. The residue was purified by column chromatography (SiO₂, Petroleum ether/Ethyl acetate=1:0 to 10:1) to give tert-butyl N-[2-[(4-bromo-2-methyl-pyrazol-3-yl)methylamino]ethyl]-N-methyl-carbamate (3.40 g, 8.52 mmol, 35.3% yield) as a yellow oil. LCMS: m/z 348.9 (M+1)⁺.

Step 2. To a mixture of tert-butyl N-[2-[(4-bromo-2-methyl-pyrazol-3-yl)methylamino]ethyl]-N-methyl-carbamate (2.74 g, 7.89 mmol, 1 eq)) in THF (80 mL) and NaHCO₃ (1.99 g, 23.7 mmol, 3 eq) in H₂O (20 mL) was added CbzCl (1.75 g, 10.3 mmol, 1.46 mL, 1.3 eq). The mixture was stirred at 20° C. for 16 h, quenched with water (80 mL) and extracted with ethyl acetate (50 mL*3). The combined organic phase was dried over anhydrous sodium sulfate, filtered and concentrated. The residue was purified by column chromatography (SiO₂, Petroleum ether/Ethyl acetate=1:0 to 4:1) to give tert-butyl N-[2-[benzyloxycarbonyl-[(4-bromo-2-methyl-pyrazol-3-yl)methyl]amino]ethyl]-N-methyl-carbamate (B-V-3, 2.89 g, 5.83 mmol, 73.9% yield) as a colorless oil. ¹H NMR (400 MHz, CDCl₃) δ (ppm) 7.42 (s, 1H), 7.36 (s, 5H), 5.18 (s, 2H), 4.67 (s, 2H), 3.84 (s, 2H), 3.70-3.23 (m, 6H), 2.79-2.67 (m, 2H), 1.45 (s, 9H).

### Example 11

### Preparation of give tert-butyl N-[2-[(4-bromo-2-methyl-pyrazo 1-3-yl)methyl-methyl-amino]ethyl]-N-methyl-carbamate (B-V-4) and tert-butyl N-methyl-N-[2-[methyl-[[2-methyl-4-(4,4,5,5-tetramethyl-1,3,2-dioxaborolan-2-yl)pyrazol-3-yl]methyl]amino]ethyl]carbamate (B-IV-4)

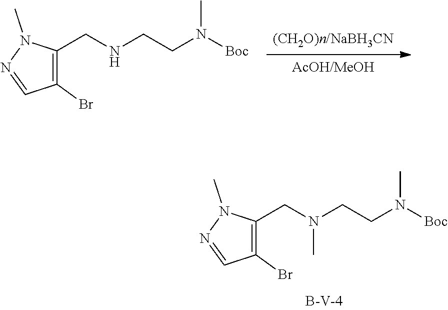 [SMILES: CN(CCNCc1c(Br)cnn1C)C(=O)OC(C)(C)C ; CN(CCN(C)C(=O)OC(C)(C)C)Cc1c(Br)cnn1C]

To a solution of tert-butyl N-[2-[(4-bromo-2-methyl-pyrazol-3-yl)methylamino]ethyl]-N-methyl-carbamate (3.30 g, 9.50 mmol, 1 eq), (CH₂O)n (1.70 g, 18.9 mmol, 1.99 eq) and AcOH (2.10 g, 34.9 mmol, 2 mL, 3.68 eq) in MeOH (80 mL) was added NaBH₃CN (716 mg, 11.4 mmol, 1.2 eq). The mixture was stirred at 20° C. for 16 h. On completion, the mixture was quenched with Sat. NH₄Cl (10 mL), concentrated in vacuum, diluted with EtOAc (100 mL) and washed with brine (2*70 mL). The organic layer was dried over sodium sulfate and concentrated in vacuum. The residue was purified by silica gel column (PE: EA=1:0-100:40) to give tert-butyl N-[2-[(4-bromo-2-methyl-pyrazo 1-3-yl)methyl-methyl-amino]ethyl]-N-methyl-carbamate (B-V-4, 3 g, 8.30 mmol, 87% yield) as colorless gum. ¹H NMR (400 MHz, CDCl₃) δ (ppm) 7.37 (s, 1H), 3.87 (s, 3H), 3.51 (s, 2H), 3.39-3.21 (m, 2H), 2.73 (s, 3H), 2.54-2.43 (m, 2H), 2.24 (s, 3H), 1.42 (s, 9H).

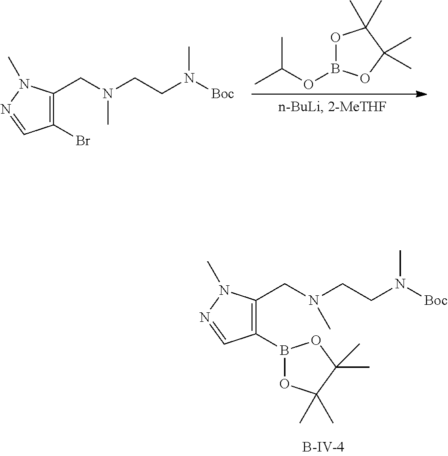

To a solution of tert-butyl N-[2-[(4-bromo-2-methyl-pyrazol-3-yl)methyl-methyl-amino]ethyl]-N-methyl-carbamate (1.10 g, 3.04 mmol, 1.0 eq) in 2-MeTHF (45.0 mL) was added n-BuLi (2.5 M, 3.04 mL, 2.5 eq) at −70° C. The mixture was stirred at −70° C. for 0.5 h, then 2-isopropoxy-4,4,5,5-tetramethyl-1,3,2-dioxaborolane (849 mg, 4.57 mmol, 931 uL, 1.5 eq) was added at this temperature and stirred at −70° C. for 1.5 h. Once completion, the mixture was quenched with Sat. NH₄Cl (50.0 mL), extracted with EtOAc (100 mL). The organic layer was washed with brine (2*25.0 mL), dried over sodium sulfate, concentrated in vacuum to afford tert-butyl N-methyl-N-[2-[methyl-[[2-methyl-4-(4,4,5,5-tetramethyl-1,3,2-dioxaborolan-2-yl)pyrazol-3-yl]methyl]amino]ethyl]carbamate (1.6 g, 2.35 mmol, 77.2% yield).

### Preparation of tert-butyl-N-[2-[benzyloxycarbonyl-[(4-bromo-3-methyl-1H-pyrazol-5-yl)methyl]amino]ethyl]-N-methyl-carbamate (B-V-5)

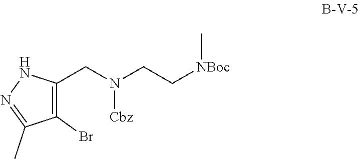 [SMILES: Cc1n[nH]c(CN(CCN(C)C(=O)OC(C)(C)C)C(=O)OCc2ccccc2)c1Br]

B-V-5 was prepared using similar procedures as B-V-3. ¹H NMR (400 MHz, DMSO-d₆) δ (ppm) 11.05 (s, 1H), 7.40-7.27 (m, 5H), 5.08 (s, 2H), 4.41 (s, 2H), 3.31 (s, 2H), 3.26 (s, 2H), 2.76 (s, 3H), 2.15 (s, 3H), 1.36 (s, 9H). LCMS: m/z 483.3 (M+1)⁺.

### Preparation of tert-butyl N-[2-[(4-bromo-5-methyl-isoxazol-3-yl) methoxy]ethyl]-N-methyl-carbamate (B-V-6)

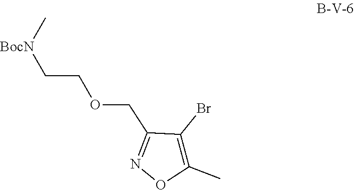 [SMILES: Cc1onc(COCCN(C)C(=O)OC(C)(C)C)c1Br]

B-V-6 was prepared using similar procedures as B-V-2 starting with (5-methylisoxazol-3-yl)methanol. ¹H NMR (400 MHz, CDCl₃) δ=4.55 (s, 2H), 3.62 (t, J=5.6 Hz, 2H), 3.41 (d, J=5.6 Hz, 2H), 2.91 (s, 3H), 2.42 (s, 3H), 1.44 (s, 9H).

### Preparation of tert-butyl N-[2-[benzyloxycarbonyl-[(4-bromo-5-methyl-isoxazol-3-yl)methyl]amino]ethyl]-N-methyl-carbamate (B-V-7)

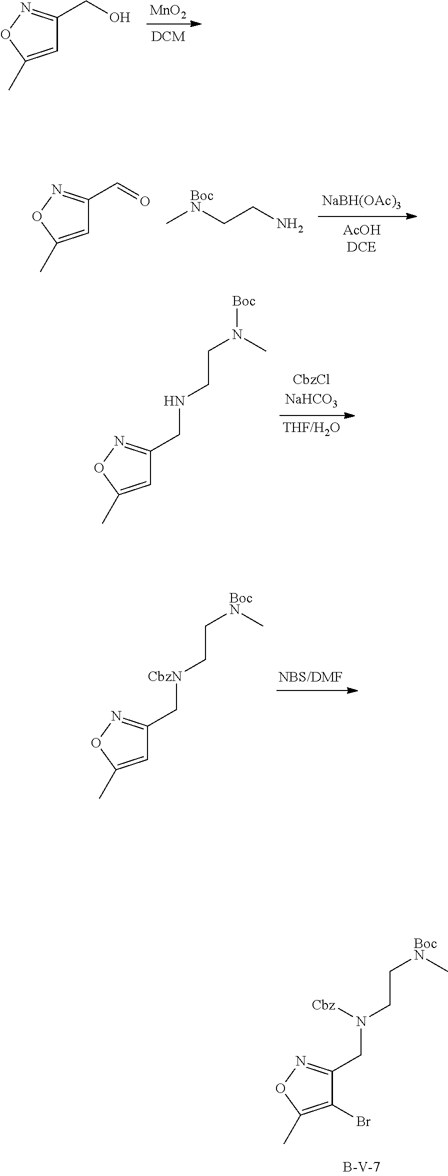

Step 1. To a solution of (5-methylisoxazol-3-yl) methanol (10.0 g, 88.0 mmol, 1 eq.) in DCM (100 mL) was added MnO₂ (38.4 g, 442 mmol, 5 eq.). The mixture was stirred at 25° C. for 16 hours and filtrated. The filtrate was concentrated in vacuum to give 5-methylisoxazole-3-carbaldehyde (6.50 g, 35.0 mmol, 39.71% yield) as yellow oil. ¹H NMR (400 MHz, CDCl₃) δ=10.12 (s, 1H), 6.40 (s, 1H) 2.53 (s, 3H).

Step 2. To a solution of 5-methylisoxazole-3-carbaldehyde (6.50 g, 58.0 mmol, 1 eq.), tert-butyl N-(2-aminoethyl)-N-methyl-carbamate (11.2 g, 64.4 mmol, 11.5 mL, 1.1 eq.) in DCE (50 mL) was added AcOH (3.50 g, 58.0 mmol, 1 eq) and NaBH(OAc)₃ (24.8 g, 117 mmol, 2 eq). The mixture was stirred at 25° C. for 16 h. On completion, 50 mL water was added, and the reaction was extracted with EtOAc (3*50 ml). The combined extracts were concentrated in vacuum. The residue was purified by column chromatography (SiO₂, DCM/MeOH, from 100:0 to 100:10) to give tert-butyl N-methyl-N-[2-[(5-methylisoxazol-3-yl)methylamino]ethyl]carbamate (1.30 g, 4.20 mmol, 7.18% yield) as colorless oil. LC-MS: m/z 270.2 (M+1)⁺.

Step 3. To a solution of tert-butyl N-methyl-N-[2-[(5-methylisoxazol-3-yl)methylam ino]ethyl]carbamate (1.20 g, 4.50 mmol, 1 eq.), benzyl carbonochloridate (912 mg, 5.30 mmol, 1.2 eq.) in THF (10 mL) and H₂O (10 mL) was added NaHCO₃ (1.10 g, 13.4 mmol, 3 eq.). The mixture was stirred at 25° C. for 16 hours. On completion, the reaction was extracted with EtOAc (3*10 ml), then concentrated in vacuum. The residue was purified by column chromatography (SiO₂, Petroleum ether/EtOAc, from 100:1 to 100:25) to give tert-butyl N-[2-[benzyloxycarbonyl-[(5-methylisoxazol-3-yl)methyl]amino]ethyl]-N-methyl-carbamate (1.20 g, 2.60 mmol, 58% yield) as colorless oil. ¹H NMR (400 MHz, CDCl₃) δ=7.28 (s, 5H), 5.91 (s, 1H), 5.09 (s, 2H), 4.47-4.41 (m, 2H), 3.38-3.22 (m, 4H), 2.80 (s, 3H), 2.32 (s, 3H), 1.36 (s, 9H); LC-MS: m/z 304.5 (M+1)⁺.

Step 4. To a solution of tert-butyl N-[2-[benzyloxycarbonyl-[(5-methylisoxazol-3-yl) meth yl]amino]ethyl]-N-methyl-carbamate (700 mg, 1.70 mmol, 1 eq.) in DMF (25 mL) was added NBS (462 mg, 2.60 mmol, 1.5 eq.). The mixture was stirred at 60° C. for 20 hours. The mixture was diluted with EtOAc (100 mL), and washed with brine (4*40 mL). The organic layer was dried over Na₂SO₄, concentrated in vacuum, purified by a silica gel column (Petroleum ether: EtOAc, from 100:0 to 100:30) to afford tert-butyl N-[2-[benzyloxycarbonyl-[(4-bromo-5-methyl-isoxazol-3-yl)methyl]amino]ethyl]-N-methyl-carbamate (B-V-7, 320 mg, 630 umol, 36.4% yield) as colorless gum. ¹H NMR (400 MHz, CDCl₃) δ=7.38-7.33 (m, 5H), 5.18 (s, 2H), 4.61-4.50 (m, 2H), 3.60-3.28 (m, 4H), 2.77-2.66 (m, 3H), 2.40 (s, 3H), 1.44 (s, 9H); LC-MS: m/z 384.3 (M-99)⁺.

### Preparation of tert-butyl N-[2-[(4-bromo-2,5-dimethyl-pyrazol-3-yl)methyl-methyl-amino]ethyl]-N-methyl-carbamate (B-V-8)

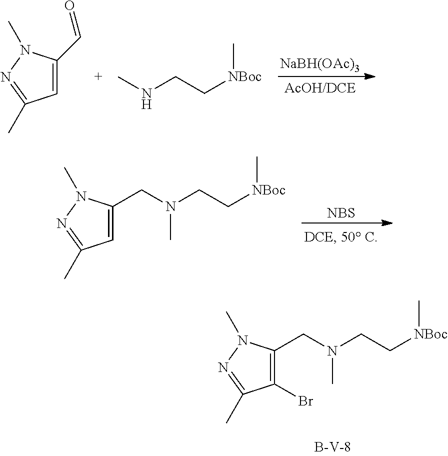

Step 1. A mixture of 2,5-dimethylpyrazole-3-carbaldehyde (2.00 g, 16.1 mmol, 1 eq), tert-butyl N-methyl-N-[2-(methylamino)ethyl]carbamate (4.55 g, 24.2 mmol, 1.5 eq) in DCE (2 mL) was added AcOH (967 mg, 16.1 mmol, 1 eq). After 0.5 hours at 25° C., NaBH(OAc)₃ (10.2 g, 48.3 mmol, 3 eq) was added at 0° C. The mixture was stirred at 25° C. for 16 hours. The mixture was quenched by pouring it into water, then extracted with EtOAc (3*50 mL). The combined organic layers were washed with brine (2*30 mL), dried over Na₂SO₄, filtered and concentrated in vacuum. The residue was purified by flash chromatography using silica gel (DCM: MeOH=25:1 to 10:1) to give tert-butyl N-[2-[(2,5-dimethylpyrazol-3-yl)methyl-methyl-amino]ethyl]-N-methyl-carbamate (1.80 g, 5.82 mmol, 36% yield) as yellow oil. 1H NMR (400 MHz, CDCl₃) δ=9.93 (s, 1H), 5.92 (s, 1H), 3.80 (s, 3H), 3.60-3.48 (m, 2H), 3.40-3.28 (m, 2H), 2.80 (s, 3H), 2.62-2.52 (m, 2H), 2.27 (s, 3H), 2.24 (s, 3H), 1.43 (s, 9H); LC-MS: m/z 297.2 (M+1)⁺.

Step 2. To a solution of tert-butyl N-[2-[(2,5-dimethylpyrazol-3-yl)methyl-methyl-amino]ethyl]-N-methyl-carbamate (1.7 g, 5.74 mmol, 1 eq) in DMF (2 mL), was added NBS (1.22 g, 6.88 mmol, 1.2 eq) at 25° C. The mixture was stirred at 60° C. for 16 hours under N₂. The reaction mixture was concentrated in vacuo. The residue was purified by flash chromatography using silica gel (DCM: MeOH=25:1-10:1) to tert-butyl N-[2-[(4-bromo-2,5-dimethyl-pyrazol-3-yl)methyl-methyl-amino]ethyl]-N-methyl-carbamate (M-V-8, 1.3 g, 3.38 mmol, 58.9% yield, 97.5% purity) as yellow oil. ¹H NMR (400 MHz, CDCl₃) δ=3.80 (s, 3H), 3.46 (s, 2H), 3.36-3.21 (m, 2H), 2.85-2.84 (m, 3H), 2.74 (s, 2H), 2.17 (s, 3H), 2.01 (s, 3H), 1.40 (s, 9H).

### Preparation of tert-butyl N-[2-[(2-bromophenyl)methyl-methyl-amino]ethyl]-N-methyl-carbamate (B-V-9)

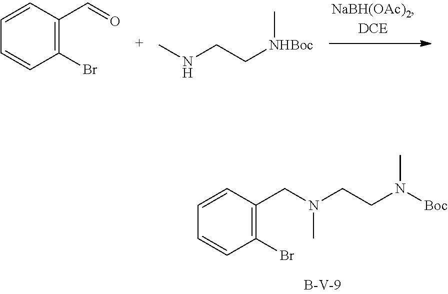

To a solution of tert-butyl N-methyl-N-[2-(methylamino)ethyl]carbamate (2.65 g, 14.0 mmol, 1.3 eq) and 2-bromobenzaldehyde (2.00 g, 10.8 mmol, 1.25 mL, 1 eq) in DCE (10 mL) was added NaBH(OAc)₃ (3.44 g, 16.2 mmol, 1.5 eq). The mixture was stirred at 25° C. for 16 hr. On completion, the mixture was quenched with water (50 mL), and extracted with EtOAc (3×50 mL). Combined organic layer was washed with brine (50 mL), dried over sodium sulfate, concentrated in vacuum, and purified by flash silica gel chromatography (40 g silica gel column, DCM in MeOH from 0% to 100%) to give tert-butyl N-[2-[(2-bromophenyl)methyl-methyl-amino]ethyl]-N-methyl-carbamate (M-V-9, 3.50 g, 8.82 mmol, 81.5% yield) as colorless gum. LC-MS: m/z 357.9 (M+1)⁺.

### Preparation of tert-butyl N-[3-(4-bromo-2,5-dimethyl-pyrazol-3-yl)oxypropyl]-N-methyl-carbamate (B-V-10)

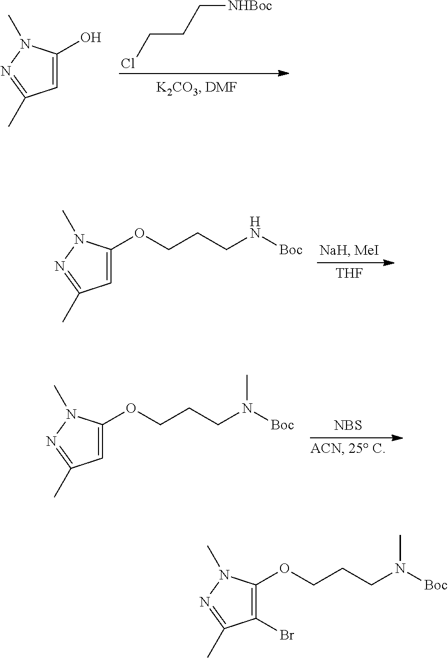

Step 1. To a solution of 2,5-dimethylpyrazol-3-ol (5 g, 44.59 mmol, 1 eq) and tert-butyl N-(3-bromopropyl)carbamate (12.74 g, 53.51 mmol, 1.2 eq) in DMF (180 mL) was added K₂CO₃ (9.24 g, 66.8 mmol, 1.50 eq). The mixture was stirred at 80° C. for 2 hrs. The reaction mixture was concentrated under reduced pressure to remove DMF. To the residue was added 1,4-dioxane (300 mL), and the mixture was filtered, washed with petro ether (30 mL*3). The filtrate was washed with brine (15 mL*3), dried over Na₂SO₄, filtered and concentrated under reduced pressure to give a tert-butyl N-(3-chloropropyl)-N-methyl-carbamate (24.5 g, crude). ¹H NMR (400 MHz, DMSO-d₆) δ=6.89 (s, 1H), 5.40 (s, 1H), 4.02-3.95 (m, 2H), 3.43 (s, 3H), 3.08-3.03 (m, 2H), 2.02 (s, 3H), 1.88-1.76 (m, 2H), 1.37 (s, 9H).

Step 2. To a solution of tert-butyl N-[3-(2,5-dimethylpyrazol-3-yl)oxypropyl]carbamate (5 g, 18.6 mmol, 1 eq) in THF (50 mL) at 0° C. under N₂ was added NaH (1.11 g, 27.8 mmol, 60% purity, 1.5 eq) at 0° C. The mixture was stirred for 0.5 hours followed by addition of Mel (3.95 g, 27.8 mmol, 1.5 eq) at 0° C. The mixture was stirred at 25° C. for 1 hour, quenched with slow addition of water, and extracted with EtOAc (3×100 mL). The combined organic layers were washed with brine (2×50 mL), dried over Na₂SO₄, filtered and concentrated in vacuo to provide tert-butyl N-[3-(2,5-dimethylpyrazol-3-yl)oxypropyl]-N-methyl-carbamate (10 g, 31.7 mmol, 85.5% yield) as yellow oil. ¹H NMR (400 MHz, DMSO-d₆) δ=5.39 (s, 1H), 4.00-3.96 (m, 2H), 3.44 (s, 3H), 3.33-3.29 (m, 2H), 2.77 (s, 3H), 2.02 (s, 3H), 1.93-1.85 (m, 2H), 1.33 (s, 9H).

Step 3. To a solution of tert-butyl N-[3-(2,5-dimethylpyrazol-3-yl)oxypropyl]-N-methyl-carbamate (6 g, 21.2 mmol, 1 eq) in ACN (30 mL) was added NBS (3.77 g, 21.2 mmol, 1 eq) at 25° C. and stirred for 16 h under N₂. The reaction mixture was concentrated in vacuo. The residue was purified by column chromatography on silica gel (PE:EA=25:1-2:1) to give tert-butyl N-[3-(4-bromo-2,5-dimethyl-pyrazol-3-yl)oxypropyl]-N-methyl-carbamate (B-V-10, 6.8 g, 18.2 mmol, 86% yield) as yellow oil. ¹H NMR (400 MHz, CDCl₃) δ=4.20 (s, 2H), 3.56 (s, 3H), 3.33 (s, 2H), 2.82 (s, 3H), 2.03 (s, 3H), 1.97-1.88 (m, 2H), 1.38 (s, 9H). LCMS: m/z 384.1 (M+Na)⁺.

### Preparation of tert-butyl N-[2-[(4-bromo-2,5-dimethyl-pyrazol-3-yl)methoxy]ethyl]-N-methyl-carbamate (B-V-11)

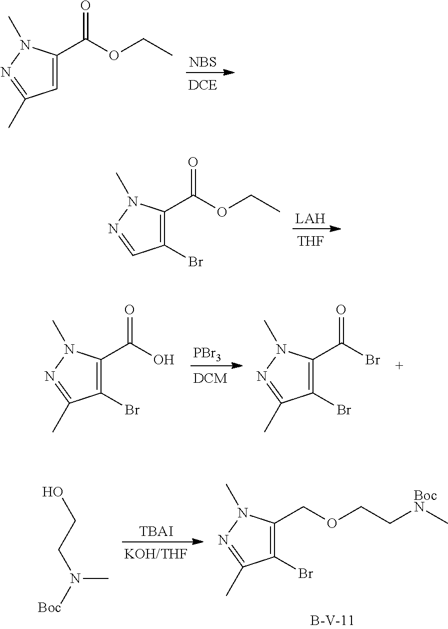

Step 1. To a solution of ethyl 2,5-dimethylpyrazole-3-carboxylate (10 g, 59.4 mmol, 1 eq) in DCE (200 mL) was added NBS (12.7 g, 71.3 mmol, 1.2 eq). The mixture was stirred at 80° C. for 16 h. The reaction mixture was concentrated in vacuo. The residue was purified by column chromatography on silica gel (PE:EA=25:1-2:1) to give ethyl 4-bromo-2,5-dimethyl-pyrazole-3-carboxylate (12 g, 45.2 mmol, 75.9% yield) as a yellow oil. ¹H NMR (400 MHz, CDCl₃) δ=4.35-4.29 (m, 2H), 4.02 (s, 3H), 2.17 (s, 3H), 1.34-1.32 (m, 3H).

Step 2. To a solution of ethyl 4-bromo-2,5-dimethyl-pyrazole-3-carboxylate (9.46 g, 38.3 mmol, 1 eq) in THF (100 mL) was added LiAlH₄ (1.60 g, 42.1 mmol, 1.1 eq). The mixture was stirred at 0° C. for 0.5 hr, and quenched by slow addition oo water (0.086 ml), aq. sodium hydroxide (15%, 1.65 mL) and water (4.8 mL). The reaction mixture was filtered and concentrated under reduced pressure to give (4-bromo-2,5-dimethyl-pyrazol-3-yl)methanol (6.5 g, 31.7 mmol, 82.8% yield) as a colorless oil. ¹H NMR (400 MHz, DMSO-d₆) a=5.31 (s, 1H), 4.44-4.40 (m, 2H), 3.79 (s, 3H), 2.22 (s, 3H).

Step 3. To a solution of (4-bromo-2,5-dimethyl-pyrazol-3-yl)methanol (6.2 g, 30.24 mmol, 1 eq) in DCM (120 mL) was added PBr₃ (8.18 g, 30.2 mmol, 1 eq) dropwise at 0-25° C. The mixture was stirred at 25° C. for 4 h, quenched by slow addition of water, and extracted with EtOA (3×100 mL). The combined organic layers were washed with brine (2×50 mL), dried over Na₂SO₄, filtered and concentrated in vacuo. The residue was purified by column chromatography on silica gel (PE: EA=25:1-3:1) to give 4-bromo-5-(bromomethyl)-1,3-dimethyl-pyrazole (6.2 g, 22.4 mmol, 74.2% yield) as a white solid. LCMS: m/z 269.0 (M+1)⁺.

Step 4. To a solution of 4-bromo-5-(bromomethyl)-1,3-dimethyl-pyrazole (4 g, 14.9 mmol, 1 eq) in THF (80 mL) were added tert-butyl N-(2-hydroxyethyl)-N-methyl-carbamate (2.88 g, 16.4 mmol, 1.1 eq), TBAI (551.40 mg, 1.49 mmol, 0.1 eq) and KOH (2.51 g, 44.8 mmol, 3 eq). The mixture was stirred at 25° C. for 16 hours under N₂. On completion, the reaction mixture was concentrated in vacuo. The residue was purified by column chromatography (SiO₂, Petroleum ether/Ethyl acetate=10/1 to 1/1) to give tert-butyl N-[2-[(4-bromo-2,5-dimethyl-pyrazol-3-yl)methoxy]ethyl]-N-methyl-carbamate (B-V-11, 5.5 g, 14.6 mmol, 97.6% yield) as yellow oil. ¹H NMR (400 MHz, CDCl₃) δ=4.50-4.46 (m, 2H), 3.83 (s, 3H), 3.63-3.31 (m, 4H), 2.87 (s, 3H), 2.22 (s, 3H), 1.44 (s, 9H).

### Preparation of tert-butyl N-[2-[(4-bromo-5-cyclopropyl-isoxazol-3-yl)methoxy]ethyl]-N-methyl-carbamate (B-V-12)

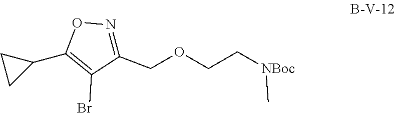 [SMILES: CN(CCOCc1noc(C2CC2)c1Br)C(=O)OC(C)(C)C]

B-V-12 was prepared using similar procedures as B-V-1 starting with 5-cyclopropylisoxazole-3-carboxylic acid. The bromonation procedure is similar as that in B-V-7. ¹H NMR (400 MHz, CDCl₃) δ=4.53 (s, 2H), 3.60 (s, 2H), 3.40 (s, 2H), 2.91 (s, 3H), 2.10-2.07 (m, 1H), 1.17 (s, 9H), 1.16-1.12 (m, 2H), 1.11-1.10 (m, 2H). LCMS: m/z 277.1 (M-Boc)⁺.

### Preparation of tert-butyl N-[2-[(4-bromo-5-isopropyl-isoxazol-3-yl)methoxy]ethyl]-N-methyl-carbamate (B-V-13)

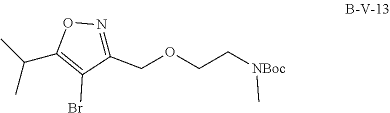 [SMILES: CC(C)c1onc(COCCN(C)C(=O)OC(C)(C)C)c1Br]

B-V-13 was prepared using similar procedures as B-V-1 starting with ethyl 5-isopropylisoxazole-3-carboxylate. The bromonation procedure is similar as that in B-V-7. ¹H NMR (400 MHz, CDCl₃) δ=4.55 (s, 2H), 3.63 (s, 2H), 3.41 (s, 2H), 2.91 (s, 3H), 1.45 (s, 9H), 1.34 (d, J=7.2 Hz, 6H). LCMS: m/z 277.1 (M-Boc)⁺.

### Preparation of tert-butyl N-[3-(4-bromo-2-methyl-pyrazol-3-yl)oxypropyl]-N-methyl-carbamate (B-V-14)

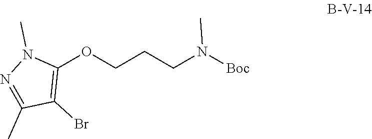 [SMILES: Cc1nn(C)c(OCCCN(C)C(=O)OC(C)(C)C)c1Br]

B-V-14 was prepared using similar procedures as B-V-10 starting with 2-methylpyrazol-3-ol. ¹H NMR (400 MHz, CDCl₃) δ=7.20 (s, 1H), 4.25 (s, 2H), 3.61 (s, 3H), 3.43-3.28 (m, 2H), 2.82 (s, 3H), 1.96-1.89 (m, 2H), 1.38 (s, 9H). LCMS: m/z 350.2 (M+1)⁺.

### Preparation of tert-butyl 3-[3-(tert-butoxycarbonylamino)propoxy]-4-[(3Z)-3-[(3-methoxycarbonyl-1H-pyrrol-2-yl)methylene]-2-oxo-1H-pyrrolo[2,3-c]pyridin-5-yl]pyrazole-1-carboxylate (C-6a) according to General Method C

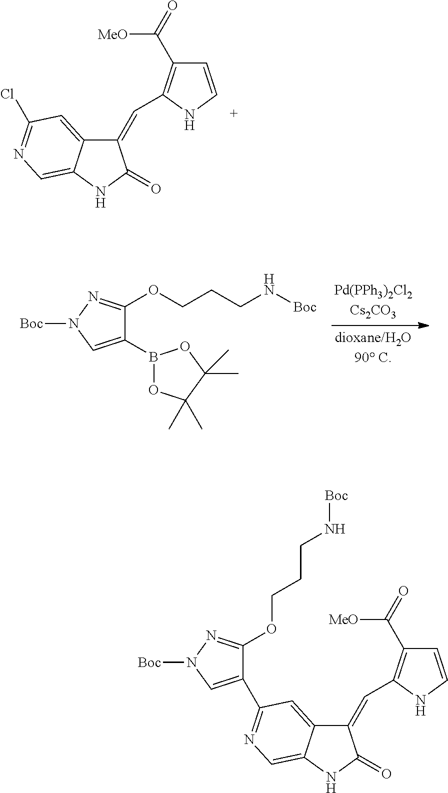 [SMILES: COC(=O)c1cc[nH]c1/C=C1\C(=O)Nc2cnc(Cl)cc21 ; CC(C)(C)OC(=O)NCCCOc1nn(C(=O)OC(C)(C)C)cc1B1OC(C)(C)C(C)(C)O1 ; COC(=O)c1cc[nH]c1/C=C1\C(=O)Nc2cnc(-c3cn(C(=O)OC(C)(C)C)nc3OCCCNC(=O)OC(C)(C)C)cc21]

To a solution of methyl 2-[(Z)-(5-chloro-2-oxo-1H-pyrrolo[2,3-c]pyridin-3-ylidene)methyl]-1H-pyrrole-3-carboxylate (500 mg, 1.65 mmol, 1 eq) and tert-butyl 3-[3-(tert-butoxycarbonylamino)propoxy]-4-(4,4,5,5-tetramethyl-1,3,2-dioxaborolan-2-yl)pyrazole-1l-carboxylate (3.85 g, 8.23 mmol, 5 eq) in dioxane (10 mL) and H₂₇ (1 mL) was added Cs₂CO₃ (1.61 g, 4.94 mmol, 3 eq) and Pd(PPh₃)₂Cl₂ (115 mg, 0.165 mmol, 0.1 eq). The resulting mixture was stirred at 90° C. for 14 h under N₂ atmosphere. On completion, the reaction mixture was concentrated in vacuum. The residue was purified by silica gel chromatography (DCM: MeOH=100:1˜ 20:1) to give C-6a (251 mg, 0.412 mmol, 25% yield) as white solid.

C-67-C₇₃ were prepared following similar procedures as C-6a.

| Comp. | | | |
|---|---|---|---|
| # | Structure | ¹NMR (400 MHz, DMSO-d₆) δ (ppm) | MS m/z |
| C-6a | 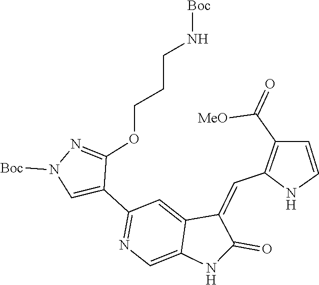 [SMILES: COC(=O)c1cc[nH]c1/C=C1\C(=O)Nc2cnc(-c3cn(C(=O)OC(C)(C)C)nc3OCCCNC(=O)OC(C)(C)C)cc21] | 13.86 (s, 1H), 11.37 (s, 1H), 8.61 (s, 1H), 8.39 (s, 1H), 8.23 (s, 1H), 8.02 (s, 1H), 7.48 (t, J = 2.4 Hz, 1H), 6.87-6.80 (m, 2H), 4.38 (t, J = 5.6 Hz, 2H), 3.86 (s, 3H), 3.28-3.26 (m, 2H), 2.07-2.00 (m, 2H), 1.59 (s, 9H), 1.34-1.27 (m, 9H) | |
| C-67 | 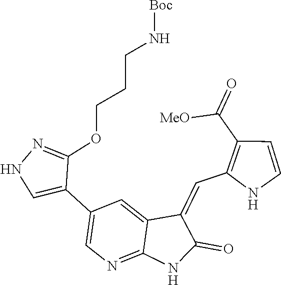 [SMILES: COC(=O)c1cc[nH]c1/C=C1\C(=O)Nc2ncc(-c3c[nH]nc3OCCCNC(=O)OC(C)(C)C)cc21] | 13.78 (s, 1H), 12.13 (s, 1H), 11.68 (s, 1H), 8.49-8.46 (m, 2H), 8.21-8.13 (dd, J = 12.4, 1.6 Hz, 2H), 7.44-7.38 (t, J = 2.8 Hz, 1H), 6.90-6.82 (t, J = 4.8 Hz, 1H), 6.81-6.76 (t, J = 2.4 Hz, 1H), 4.32-4.21 (t, J = 6.0 Hz, 2H), 3.87 (s, 3H), 3.23-3.13 (m, 2H), 2.02- 1.87 (m, 2H), 1.35 (s, 9H) | 509.1 (M + 1)⁺ |
| C-68 | 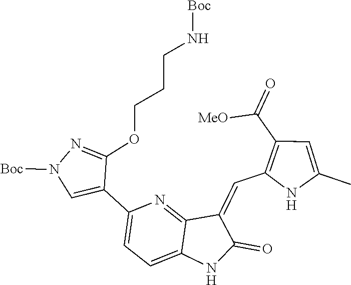 [SMILES: COC(=O)c1cc(C)[nH]c1/C=C1\C(=O)Nc2ccc(-c3cn(C(=O)OC(C)(C)C)nc3OCCCNC(=O)OC(C)(C)C)nc21] | 14.49 (s, 1H), 10.83 (s, 1H), 8.69 (s, 1H), 8.37 (s, 1H), 7.70 (d, J = 8.0 Hz, 1H), 7.43 (d, J = 8.0 Hz, 1H), 6.94- 6.92 (m, 1H), 6.65 (s, 1H), 4.36 (t, J = 6.0 Hz, 2H), 3.84 (s, 1H), 3.21-3.04 (m, 2H), 2.52 (s, 3H), 1.98-1.87 (m, 2H), 1.63 (s, 9H), 1.37 (s, 9H) | 623.3 (M + 1)⁺ |
| C-69 | 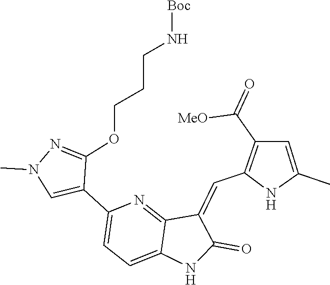 | 14.60-14.53 (s, 1H), 10.65 (s, 1H), 8.31 (s, 1H), 8.10 (s, 1H), 7.56 (d, J = 12 Hz, 1H), 7.35-7.31 (m, J = 8.4 Hz 1H), 6.93-6.86 (t, J = 4.8 Hz, 1H), 6.63-6.59 (d, J = 2.4 Hz, 1H), 4.24 (t, J = 6.4 Hz, 2H), 3.83-3.79 (m, 3H), 3.78-3.73 (m, 3H), 3.12-3.05 (m, 2H), 2.47 (s, 3H), 1.89-1.82 (m, 2H), 1.37 (s, 9H) | 537.4 (M + 1)⁺ |
| C-70 | 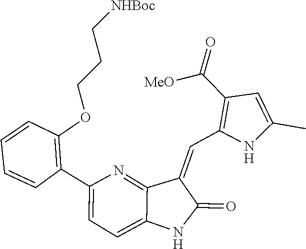 [SMILES: COC(=O)c1cc(C)[nH]c1/C=C1\C(=O)Nc2ccc(-c3ccccc3OCCCNC(=O)OC(C)(C)C)nc21] | 14.98 (s, 1H), 10.81 (s, 1H), 8.34 (s, 1H), 7.78-7.69 (m, 2H), 7.47-7.42 (m, 1H), 7.39 (d, J = 8.4 Hz, 1H), 7.21- 7.08 (m, 2H), 6.90-6.79 (m, 1H), 6.58 (d, J = 1.6 Hz, 1H), 4.07-4.04 (m, 2H), 3.81 (s, 3H), 3.07-2.97 (m, 2H), 2.23 (s, 3H), 1.77 (t, J = 6.4 Hz, 2H), 1.35 (s, 9H). | 533.5 (M + 1)⁺ |
| C-71 | 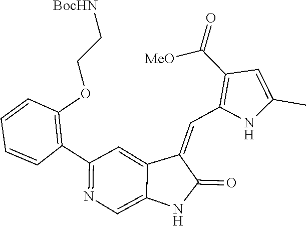 [SMILES: COC(=O)c1cc(C)[nH]c1/C=C1\C(=O)Nc2cnc(-c3ccccc3OCCNC(=O)OC(C)(C)C)cc21] | | 519.3 (M + 1)⁺ |
| C-72 | 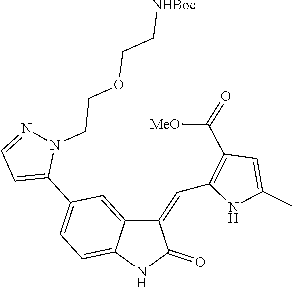 [SMILES: COC(=O)c1cc(C)[nH]c1/C=C1\C(=O)Nc2ccc(-c3ccnn3CCOCCNC(=O)OC(C)(C)C)cc21] | 13.80 (s, 1H), 11.27 (s, 1H), 8.34 (s, 1H), 7.56 (s, 1H), 7.52 (d, J = 2.0 Hz, 1H), 7.36 (t, J = 8.0 Hz, 1H), 7.03 (d, J = 8.0 Hz, 1H), 6.58-6.53 (m, 1H), 6.35 (d, J = 2.0 Hz, 1H), 4.19 (d, J = 5.2 Hz, 2H), 3.82-3.79 (m, 2H), 3.78 (s, 3H), 3.29 (t, J = 6.0 Hz, 2H), 2.99 (d, J = 5.6 Hz, 2H), 2.34 (s, 3H), 1.29 (s, 9H) | 536.2 (M + 1)⁺ |
| C-73 | 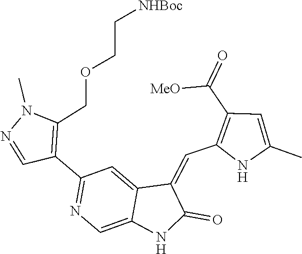 [SMILES: COC(=O)c1cc(C)[nH]c1/C=C1\C(=O)Nc2cnc(-c3cnn(C)c3COCCNC(=O)OC(C)(C)C)cc21] | 14.02 (s, 1H), 11.32 (s, 1H), 8.53 (s, 1H), 8.21 (s, 1H), 7.95 (s, 1H), 7.71 (s, 1H), 6.80-6.76 (m, 1H), 6.64 (s, 1H), 4.92 (s, 2H), 3.88 (s, 3H), 3.85 (s, 3H), 3.49 (t, J = 5.8 Hz, 2H), 3.15-3.11 (m, 2H), 2.39 (s, 3H), 1.33 (s, 9H) | |

### Preparation of 3a(4)Z]-9,10,11,12-tetrahydro-14H-17,1-(azenometheno)pyrazolo[4,3-n]dipyrrolo[13,2-g:3′,4′-j][11,5]oxazacyclopentadecine-3,8(2H,5H)-dione (6) according to General Method J

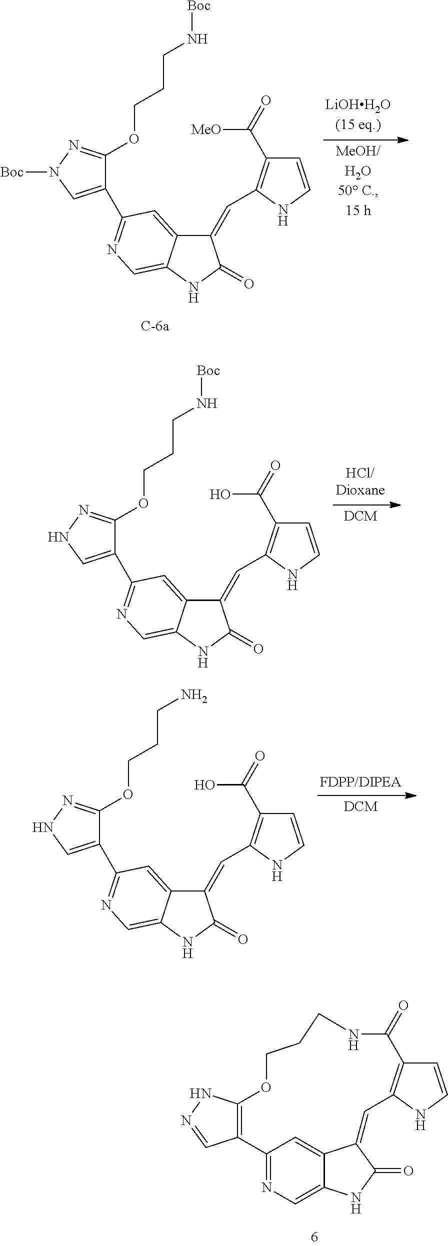

Step 1. To a solution of tert-butyl 3-[3-(tert-butoxycarbonylamino)propoxy]-4-[(3Z)-3-[(3-methoxycarbonyl-1H-pyrrol-2-yl)methylene]-2-oxo-1H-pyrrolo[2,3-c]pyridin-5-yl]pyrazole-1-carboxylate (200 mg, 0.329 mmol, 1 eq) in MeOH (4 mL) and H₂O (0.4 mL) was added LiOH·H₂O (206 mg, 4.93 mmol, 15 eq). The resulting mixture was stirred at 50° C. for 15 h. On completion, the mixture was concentrated in vacuo, and dissolved in water (300 ml), the aqueous phase was adjusted pH to 5-6 with 1M aq·HCl to precipitate the product. The solid was filtered and triturated with MeOH (15 mL) at 25° C. for 5 min to give 2-[(Z)-[5-[3-[3-(tert-butoxycarbonylamino)propoxy]-1H-pyrazol-4-yl]-2-oxo-1H-pyrrolo[2,3-c]pyridin-3-ylidene]methyl]-1H-pyrrole-3-carboxylic acid (154 mg, 0.311 mmol, 95% yield) as an orange solid. LCMS m/z 495.2 (M+1)⁺.

Step 2. The mixture of 2-[(Z)-[5-[3-[3-(tert-butoxycarbonylamino)propoxy]-1H-pyrazol-4-yl]-2-oxo-1H-pyrrolo[2,3-c]pyridin-3-ylidene]methyl]-1H-pyrrole-3-carboxylic acid (154 mg, 0.311 mmol, 1 eq) and HCl/dioxane (4 M, 0.778 mL, 10 eq) in DCM (2 mL) was stirred at 25° C. for 2 h. On completion, the mixture was concentrated in vacuo to obtain 2-[(Z)-[5-[3-(3-aminopropoxy)-1H-pyrazol-4-yl]-2-oxo-1H-pyrrolo[2,3-c]pyridine-3-ylidene]methyl]-1H-pyrrole-3-carboxylic acid HCl salt (130 mg) as a red solid. ¹H NMR (400 MHz, DMSO-d₆) δ (ppm) 13.99-13.83 (m, 1H), 12.92-12.66 (m, 1H), 11.87-11.72 (m, 1H), 8.93-8.81 (m, 1H), 8.68-8.53 (m, 1H), 8.16 (s, 2H), 7.97-7.80 (m, 3H), 7.70-7.65 (m, 1H), 6.97-6.91 (m, 1H), 4.48-4.42 (m, 2H), 3.19-3.14 (m, 2H), 2.23-2.16 (m, 2H).

Step 3. To a solution of 2-[1(Z)-[5-[3-(3-aminopropoxy)-1H-pyrazol-4-yl]-2-oxo-1H-pyrrolo [2,3-c]pyridin-3-ylidene]methyl]-1Hpyrrole-3-carboxylic acid (70 mg, HCl) in DMF (3.5 mL) was added DIPEA (114 mg, 0.887 mmol, 0.154 mL, 5 eq) and FDPP (136 mg, 0.355 mmol, 2 eq). The mixture was stirred at 20° C. for 0.5 h. On completion, the reaction was quenched with H₂O (30 mL) and filtered. The filtered cake was concentrated in vacuum to give the crude product, which was then triturated with MeOH (2 mL), filtered and dried in vacuum to give 6 as a yellow solid (23.4 mg, 32.5% yield). ¹H NMR (400 MHz, DMSO-d₆) δ (ppm) 13.58 (s, 1H), 12.15 (s, 1H), 11.12 (s, 1H), 8.94 (s, 1H), 8.54-8.47 (m, 1H), 8.11 (s, 2H), 8.03 (d, J=1.6 Hz, 1H), 7.35 (t, J=2.4 Hz, 1H), 6.83 (s, 1H), 4.43 (t, J=6.4 Hz, 2H), 3.75 (s, 2H), 2.22 (s, 2H); LCMS m/z 377.4 (M+1)⁺.

Examples 7, 11, 14, 22, 24, 39 and 123 were prepared following similar procedures as 6.

| Ex | | ¹H NMR (400 MHz, DMSO-d₆) □ | |
|---|---|---|---|
| # | Structure | ppm | MS m/z |
| 6 | 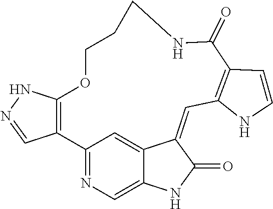 [SMILES: O=C1Nc2cnc3cc2/C1=C/c1[nH]ccc1C(=O)NCCCOc1[nH]ncc1-3] | 13.58 (s, 1H), 12.15 (s, 1H), 11.12 (s, 1H), 8.94 (s, 1H), 8.54-8.47 (m, 1H), 8.11 (s, 2H), 8.03 (d, J = 1.6 Hz, 1H), 7.35 (t, J = 2.4 Hz, 1H), 6.83 (s, 1H), 4.43 (t, J = 6.4 Hz, 2H), 3.75 (s, 2H), 2.22 (s, 2H) | 377.4 (M + 1)⁺ |
| 7 | 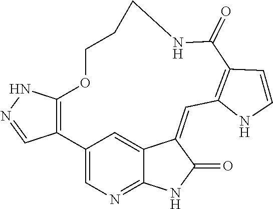 [SMILES: O=C1Nc2ncc3cc2/C1=C/c1[nH]ccc1C(=O)NCCCOc1[nH]ncc1-3] | 13.35 (s, 1H), 12.15 (s, 1H), 11.16 (s, 1H), 8.76 (s, 1H), 8.47-8.39(m, 3H), 8.20 (s, 1H), 7.30-7.24(m, 1H), 6.78- 6.74(m, 1H), 4.46-4.35 (t, J = 6.0 Hz, 2H), 3.74-3.73 (m, 2H), 2.18-2.17 (m, 2H) | 377.3 (M + 1)⁺ |
| 11 | 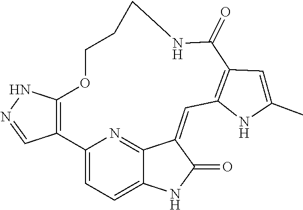 [SMILES: Cc1cc2c([nH]1)/C=C1\C(=O)Nc3ccc(nc31)-c1cn[nH]c1OCCCNC2=O] | 12.37 (s, 1H), 12.30 (s, 1H), 10.89 (s, 1H), 8.56 (s, 1H), 8.40 (s, 1H), 8.12 (s, 1H), 7.36 (d, J = 8.0 Hz, 1H), 7.20 (d, J = 8.0 Hz, 1H), 6.35 (s, 1H), 4.59 (t, J = 7.2 Hz, 2H), 3.20-3.16 (m, 2H), 2.40 (s, 3H), 2.02-1.98 (m, 2H) | 391.1 (M + 1)⁺ |
| 14 | 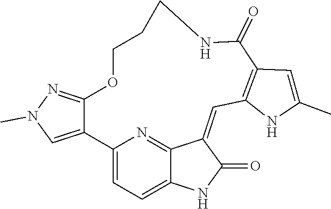 | 13.42 (s, 1H), 10.86 (s, 1H), 8.07 (s, 1H), 8.00 (s, 1H), 7.64 (s, 1H), 7.32 (d, J = 6.8 Hz, 1H), 7.23-7.11 (m, 1H), 5.43 (m, 1H), 4.44 (m, 2H), 3.74 (s, 3H), 3.67 (m, 2H), 2.21 (s, 3H), 1.80 (s, 2H). | 405.5 (M + 1)⁺ |
| 22 | 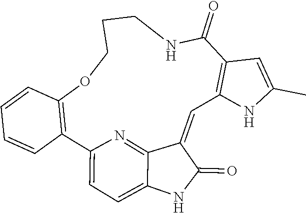 [SMILES: Cc1cc2c([nH]1)/C=C1\C(=O)Nc3ccc(nc31)-c1ccccc1OCCCNC2=O] | 12.26 (s, 1H), 11.02 (s, 1H), 8.39 (s, 1H), 8.29-8.20 (m, 1H), 7.61-7.54 (m, 1H), 7.47-7.35 (m, 2H), 7.35- 7.25 (m, 2H), 7.23-7.16 (m, 1H), 6.36 (s, 1H), 4.66-4.53 (m, 2H), 3.30- 3.26 (m, 2H), 2.42 (s, 3H), 2.17-2.01 (m, 2H). | 401.0 (M + 1)⁺ |
| 24 | 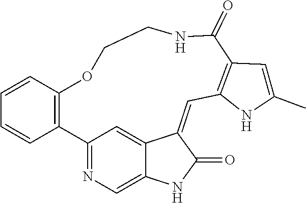 [SMILES: Cc1cc2c([nH]1)/C=C1\C(=O)Nc3cnc(cc31)-c1ccccc1OCCNC2=O] | 13.45 (s, 1H), 11.12 (s, 1H), 8.49 (s, 1H), 8.29 (s, 1H), 7.92 (s, 1H), 7.90- 7.83 (dd, J = 7.6, 1.6 Hz, 1H), 7.75- 7.67 (m, 1H), 7.41-7.35 (m, 1H), 7.32-7.24 (m, 1H), 7.11 (t, J = 3.6 Hz, 1H), 5.92 (d, J = 2.0 Hz, 1H), 4.32-4.23 (m, 2H), 3.76-3.66 (m, 2H), 3.25 (s, 3H), 2.26 (s, 2H) | 387.4 (M + 1)⁺ |
| 39 | 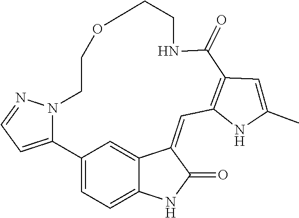 [SMILES: Cc1cc2c([nH]1)/C=C1\C(=O)Nc3ccc(cc31)-c1ccnn1CCOCCNC2=O] | 13.42 (s, 1H), 10.89 (s, 1H), 7.91 (t, J = 5.6 Hz, 1H), 7.78 (s, 1H), 7.48 (s, 1H), 7.23 (s, 1H), 7.19 (d, J = 8.0 Hz, 1H), 6.52 (d, J = 8.0 Hz, 1H), 6.13 (s, 1H), 6.03 (s, 1H), 4.16 (t, J = 4.0 Hz, 2H), 3.94 (t, J = 4.0 Hz, 2H), 3.43-3.40 (m, 2H), 3.38-3.29 (m, 2H), 2.31 (s, 3H). | 404.2 (M + 1)⁺ |
| 123 | 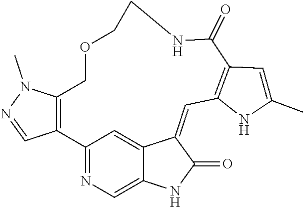 [SMILES: Cc1cc2c([nH]1)/C=C1\C(=O)Nc3cnc(cc31)-c1cnn(C)c1COCCNC2=O] | 8.39 (s, 1H), 8.17-8.12 (m, 1H), 8.10 (s, 1H), 7.84 (s, 1H), 7.49 (s, 1H), 6.16-6.12 (m, 1H), 4.77 (s, 2H), 3.93 (s, 3H), 3.88 (d, J = 5.4 Hz, 2H), 3.64 (d, J = 4.8 Hz, 2H), 2.24 (s, 3H). | 405.2 (M + 1)⁺ |

### Preparation of tert-butyl N-methyl-N-[12-[12-[5-(2-oxoindolin-5-yl) pyrazol-1-yl]ethoxy]ethyl]carbamate (K-1) according to General Method K

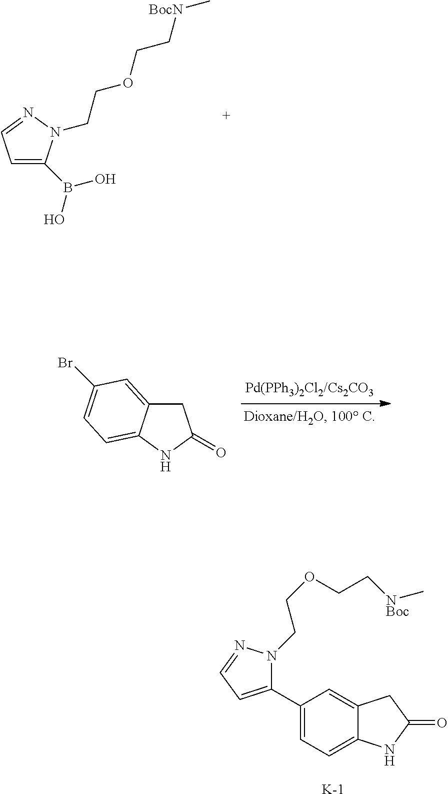

To a mixture of [12-[2-[2-tert-butoxycarbonyl(methyl)amino]ethoxy]ethyl]pyrazol-3-yl]boronic acid (588 mg, 1.88 mmol, 1 eq), 5-bromoindolin-2-one (517 mg, 2.44 mmol, 1.3 eq), and Cs₂CO₃ (1.84 g, 5.63 mmol, 3 eq) in dioxane (10 mL) and H₂O (2 mL) was added Pd(PPh₃)₂Cl₂ (131 mg, 0.187 mmol, 0.1 eq) under nitrogen. The mixture was stirred at 100° C. for 16 h under N₂, then cooled and concentrated in vacuum. The residue was purified by column chromatography (SiO₂, DCM/MeOH=30/1 to 10/1) to give tert-butyl N-methyl-N-[2-[2-[5-(2-oxoindolin-5-yl) pyrazol-1-yl]ethoxy]ethyl]carbamate (K-1, 150 mg, 17% yield) as a yellow oil. ¹H NMR (400 MHz, CDCl₃) δ (ppm) 8.17 (s, 1H), 7.58 (d, J=6.8 Hz, 1H), 7.41-7.35 (m, 2H), 6.96 (d, J=2.4 Hz, 1H), 6.25 (d, J=1.6 Hz, 1H), 4.27 (t, J=5.6 Hz, 2H), 3.92 (t, J=5.6 Hz, 2H), 3.61 (s, 2H), 3.48 (s, 2H), 3.30 (d, J=5.6 Hz, 2H), 2.78 (s, 3H), 1.42 (s, 9H). LCMS: m/z 401.0 (M+1)⁺.

K-2 was prepared following similar procedures as K-1

| Comp. | | | |
|---|---|---|---|
| # | Structure | ¹NMR (400 MHz, DMSO-d₆) δ (ppm) | MS m/z |
| K-2 | 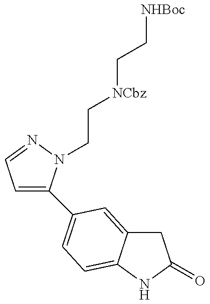 [SMILES: CC(C)(C)OC(=O)NCCN(CCn1nccc1-c1ccc2c(c1)CC(=O)N2)C(=O)OCc1ccccc1] | | 520.1 (M + 1)⁺ |

### Preparation of afford tert-butyl N-methyl-N-[2-[[2-methyl-4-(2-oxoindolin-5-yl) pyrazol-3-yl]methoxy]ethyl]carbamate (L-1) according to General Method L

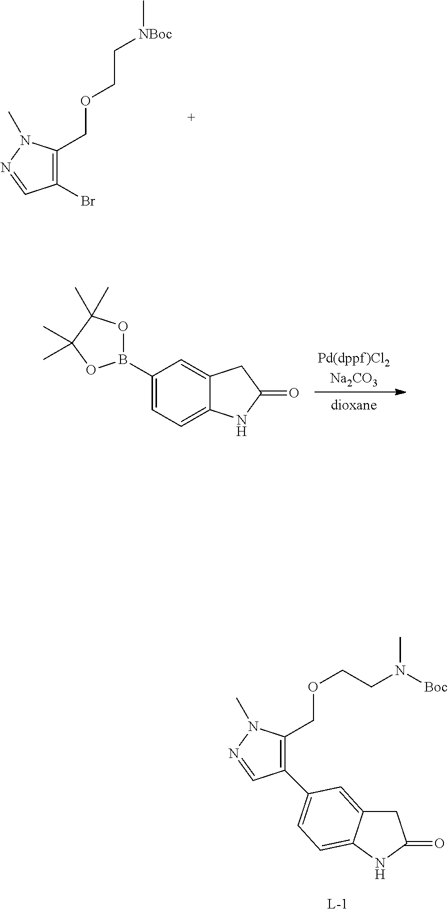 [SMILES: CN(CCOCc1c(Br)cnn1C)C(=O)OC(C)(C)C ; CC1(C)OB(c2ccc3c(c2)CC(=O)N3)OC1(C)C ; CN(CCOCc1c(-c2ccc3c(c2)CC(=O)N3)cnn1C)C(=O)OC(C)(C)C]

To a solution of tert-butyl N-[2-[(4-bromo-2-methyl-pyrazol-3-yl)methoxy]ethyl]-N-methyl-carbamate (600 mg, 1.72 mmol, 1 eq), 5-(4,4,5,5-tetramethyl-1,3,2-dioxaborolan-2-yl)indolin-2-one (668 mg, 2.58 mmol, 1.5 eq) in dioxane (17 mL) was added Pd(dppf)Cl₂ (125 mg, 0.172 mmol, 0.1 eq) and aqueous Na₂CO₃ (2 M, 2.58 mL, 3.0 eq) under nitrogen. The mixture was stirred at 100° C. for 2 h under nitrogen atmosphere. On completion, the mixture was concentrated in vacuum to afford the residue. The residue was purified by silica gel column (DCM: MeOH=100: 0-100:5) to afford tert-butyl N-methyl-N-2-[[12-methyl-4-(2-oxoindolin-5-yl) pyrazol-3-yl]methoxy]ethyl]carbamate (L-1, 600 mg, 1.50 mmol, 87% yield) as light brown gum. LCMS: m/z 401.2 (M+1)⁺.

L-2-L-13 were prepared following similar procedures as L-1.

| Comp. | | ¹NMR (400 MHz, DMSO-d₆) δ | |
|---|---|---|---|
| # | Structure | (ppm) | MS m/z |
| L-1 | 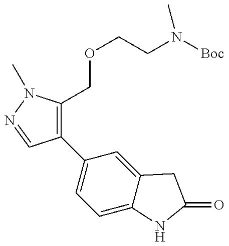 [SMILES: CN(CCOCc1c(-c2ccc3c(c2)CC(=O)N3)cnn1C)C(=O)OC(C)(C)C] | | 401.2 (M + 1)⁺ |
| L-2 | 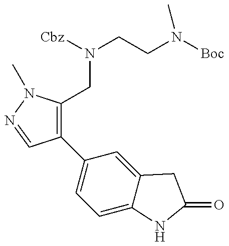 [SMILES: CN(CCN(Cc1c(-c2ccc3c(c2)CC(=O)N3)cnn1C)C(=O)OCc1ccccc1)C(=O)OC(C)(C)C] | 7.60-7.45 (m, 2H), 7.34 (s, 4H), 7.22-7.07 (m, 2H), 6.86 (s, 1H), 5.14 (s, 2H), 4.86-4.72 (m, 2H), 3.92-3.67 (m, 3H), 3.56 (s, 2H), 3.17-2.91 (m, 4H), 2.58-2.58 (m, 1H), 2.50-2.05 (m, 2H), 1.41 (s, 9H) | 534.2 (M + 1)⁺ |
| L-3 | 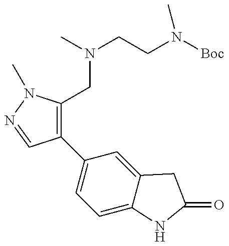 [SMILES: CN(CCN(C)C(=O)OC(C)(C)C)Cc1c(-c2ccc3c(c2)CC(=O)N3)cnn1C] | 10.37 (s, 1H), 7.47 (s, 1H), 7.25 (s, 1H), 7.23 (d, J = 8.0 Hz, 1H), 6.83 (d, J = 8.0 Hz, 1H), 3.83 (s, 3H), 3.49 (s, 2H), 3.33 (s, 3H), 3.25-3.19 (m, 2H), 2.41-2.32 (m, 2H), 2.10 (s, 1H), 1.32 (s, 9H) | 414.3 (M + 1)⁺ |
| L-4 | 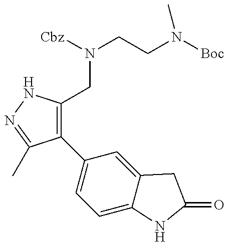 [SMILES: Cc1n[nH]c(CN(CCN(C)C(=O)OC(C)(C)C)C(=O)OCc2ccccc2)c1-c1ccc2c(c1)CC(=O)N2] | 12.50 (s, 1H), 10.36 (s, 1H), 7.41- 7.22 (m, 5H), 7.18-7.09 (m, 1H), 7.03 (s, 1H), 6.78-6.76 (m, 1H), 4.99 (s, 1H), 4.89 (s, 1H), 4.45 (s, 2H), 3.43 (s, 2H), 3.25 (s, 3H), 2.75- 2.64 (m, 2H), 2.61-2.59 (m, 2H), 2.17 (s, 3H) | 534.7 (M + 1)⁺ |
| L-5 | 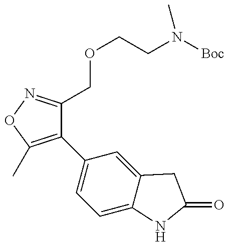 [SMILES: Cc1onc(COCCN(C)C(=O)OC(C)(C)C)c1-c1ccc2c(c1)CC(=O)N2] | 7.64 (s, 1H), 7.26-7.22 (m, 2H), 6.94 (d, J = 8.0 Hz, 1H), 4.51 (s, 2H), 3.60 (s, 4H), 3.37 (s, 2H), 2.84 (s, 3H), 2.45 (s, 3H), 1.43 (s, 9H) | 302.0 (M + 1)⁺ |
| L-6 | 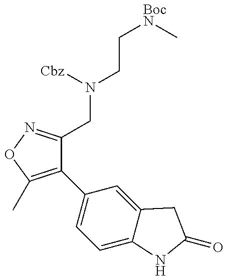 [SMILES: Cc1onc(CN(CCN(C)C(=O)OC(C)(C)C)C(=O)OCc2ccccc2)c1-c1ccc2c(c1)CC(=O)N2] | 10.46 (s, 1H), 7.40-7.13 (m, 5H), 7.13-6.96 (m, 2H), 6.84-6.82 (m, 1H), 5.05-4.79 (m, 2H), 4.52 (d, J = 10.0 Hz, 2H), 3.46 (s, 1H), 3.40 (s, 1H), 3.32 (s, 3H), 3.28-3.23 (m, 2H), 2.73 (s, 1H), 2.63 (s, 1H), 2.35 (d, J = 8.0 Hz, 3H), 1.32 (s, 9H). | 535.2 (M + 1)⁺ |
| L-7 | 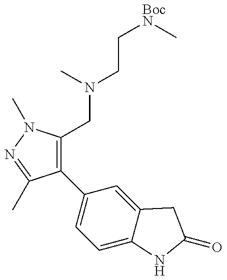 [SMILES: Cc1nn(C)c(CN(C)CCN(C)C(=O)OC(C)(C)C)c1-c1ccc2c(c1)CC(=O)N2] | | 428.3 (M + 1)⁺ |
| L-8 | 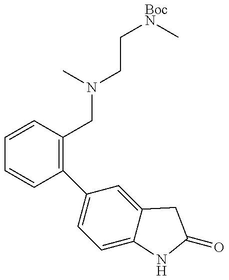 [SMILES: CN(CCN(C)C(=O)OC(C)(C)C)Cc1ccccc1-c1ccc2c(c1)CC(=O)N2] | | 410.2 (M + 1)⁺ |
| L-9 | 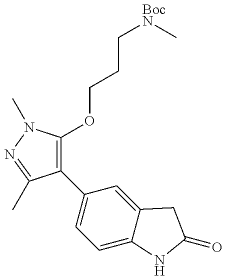 [SMILES: Cc1nn(C)c(OCCCN(C)C(=O)OC(C)(C)C)c1-c1ccc2c(c1)CC(=O)N2] | ¹H NMR (400 MHz, CDCl₃) δ = 8.01 (s, 1H), 7.22-7.18 (m, 2H), 6.89 (d, J = 8.0 Hz, 1H), 3.83-3.80 (m, 2H), 3.70 (s, 3H), 3.58 (s, 2H), 3.27 (s, 2H), 2.79 (s, 3H), 2.21 (s, 3H), 1.83- 1.77 (m, 2H), 1.41 (s, 9H). | 415.2 (M + 1)⁺ |
| L-10 | 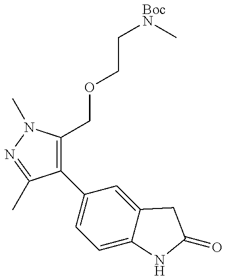 [SMILES: Cc1nn(C)c(COCCN(C)C(=O)OC(C)(C)C)c1-c1ccc2c(c1)CC(=O)N2] | ¹H NMR (400 MHz, CDCl₃) δ = 8.09 (s, 1H), 7.20-7.06 (m, 2H), 6.92 (d, J = 7.8 Hz, 1H), 4.41 (s, 2H), 3.90 (s, 3H), 3.59-3.57 (m, 2H), 3.56-3.48 (m, 2H), 3.40-3.29 (m, 2H), 2.88 (s, 3H), 2.25 (s, 3H), 1.57-1.33 (m, 9H). | 415.3 (M + 1)⁺ |
| L-11 | 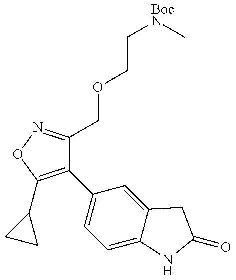 [SMILES: CN(CCOCc1noc(C2CC2)c1-c1ccc2c(c1)CC(=O)N2)C(=O)OC(C)(C)C] | ¹H NMR (400 MHz, CDCl₃) δ = 7.72 (s, 1H), 7.35-7.33 (m, 2H), 6.92 (d, J = 8.2 Hz, 1H), 4.48 (s, 2H), 3.59- 3.50 (m, 4H), 3.40-3.37 (m, 2H), 2.84 (s, 3H), 2.05-2.03 (m, 1H), 1.43 (s, 9H), 1.26-1.25 (m, 2H), 1.15-1.13 (m, 2H). | 328.2 (M − Boc)⁺ |
| L-12 | 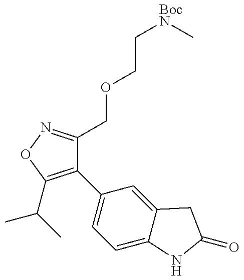 [SMILES: CC(C)c1onc(COCCN(C)C(=O)OC(C)(C)C)c1-c1ccc2c(c1)CC(=O)N2] | ¹H NMR (400 MHz, CDCl₃) δ = 7.77 (s, 1H), 7.20-7.18 (m, 2H), 6.92 (d, J = 8.0 Hz, 1H), 4.47 (s, 2H), 3.59- 3.50 (m, 4H), 3.40-3.37 (m, 2H), 3.20-3.14 (m, 1H), 2.84 (s, 3H), 1.45 (s, 9H), 1.34 (d, J = 7.2 Hz, 6H). | 330.4 (M − Boc)⁺ |
| L-13 | 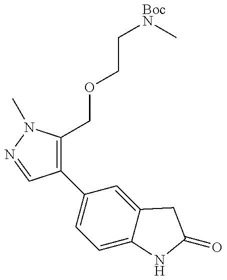 [SMILES: CN(CCOCc1c(-c2ccc3c(c2)CC(=O)N3)cnn1C)C(=O)OC(C)(C)C] | ¹H NMR (400 MHz, CDCl₃) δ = 8.63 (s, 1H), 7.49 (s, 1H), 7.39-7.27 (m, 2H), 6.82 (d, J = 6.8 Hz, 1H), 4.25- 4.11 (m, 2H), 3.91 (s, 3H), 3.43- 3.28 (m, 2H), 3.34 (s, 2H), 2.82 (s, 3H), 1.96-1.89 (m, 2H), 1.38 (s, 9H). | 401.2 (M + 1)⁺ |

### Preparation of Preparation of tert-butyl N-[12-(2-hydroxyethoxy)ethyl]-N-methyl-carbamate (M)

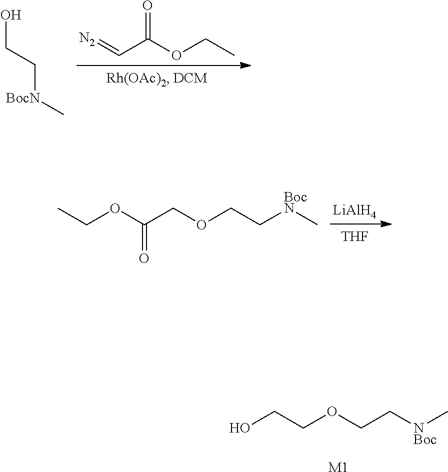

Step 1. To a mixture of tert-butyl N-(2-hydroxyethyl)-N-methyl-carbamate (5.0 g, 28.5 mmol, 1 eq) and Rh(OAc)₂ (315 mg, 1.43 mmol, 0.05 eq) in DCM (80 mL) was added a solution of ethyl 2-diazoacetate (9.77, 85.6 mmol, 3 eq) in DCM (50 mL) dropwise. The mixture was stirred at 25° C. for 16 hours and partitioned by addition of H₂O (5 mL). The organic phase was separated, washed with H₂O (10 mL*3), dried over Na₂SO₄, filtered and concentrated under reduced pressure to afford ethyl 2-[2-[tert-butoxycarbony l(methyl)amino]ethoxy]acetate (13.0 g, crude) as a yellow oil. ¹H NMR (400 MHz, CDCl₃) δ−4.23 (d, J=2.4 Hz, 2H), 4.09-4.05 (m, 2H), 3.66 (br s, 2H), 3.49-3.41 (m, 2H), 2.93 (s, 3H), 1.45 (s, 9H), 1.30-1.27 (in, 3H).

Step 2. To a solution of ethyl 2-[2-I[tert-butoxycarbonyl(methyl)amino]ethoxy]acetate (6.00 g, 22.9 mmol, 1 eq) in THF (60 mL) was added LiAlH₄ (1.31 g, 34.4 mmol, 1.5 eq) at 0° C. under N₂. The mixture was stirred at 25° C. for 2 hours. On completion, the mixture was quenched with water (1 mL) followed by addition of aq. NaOH (15%, 3 mL) and H₂O (3 mL). Na₂SO₄ was added to the combined mixture followed by stirring for 10 min. The mixture was filtered and concentrated in vacuum. The residue was purified by column chromatography (SiO₂, Petroleum ether/Ethyl acetate=8/1 to 4/1) to afford tert-butyl N-[2-(2-hydroxyethoxy)ethyl]-N-methyl-carbamate (3.00 g, 13.7 mmol, 60% yield) as a light yellow oil. ¹H NMR (400 MHz, CDCl₃) δ=3.73-3.68 (m, 2H), 3.63-3.55 (m, 4H), 3.41 (d, J=5.2 Hz, 2H), 2.90 (s, 3H), 2.31 (s, 1H), 1.45 (s, 9H)

### Preparation of Preparation of tert-butyl N-[2-[(2R)-2-hydroxypropoxy]ethyl]-N-methyl-carbamate (M5)

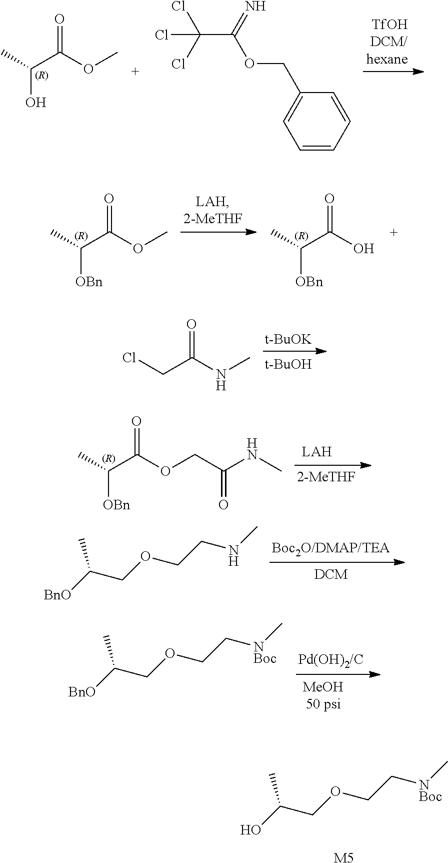

Step 1. To the mixture of methyl (2R)-2-hydroxypropanoate (20.0 g, 192 mmol, 1 eq.) and benzyl 2,2,2-trichloroethanimidate (51.0 g, 202 mmol, 1.05 eq.) in the solution of DCM (66.5 mL) and Hexane (133 mL) was added trifluoromethanesulfonic acid (1.11 mL) dropwise at 0° C. The mixture was stirred at 20° C. for 50 hours, and then filtered. The filtrate was concentrated in vacuum, and the residue was purified by silica gel column (Petroleum ether: EtOAc, from 100:1 to 100:3) to afford methyl (2S)-2-benzyloxypropanoate (8.00 g, 37.0 mmol, 19% yield) as colorless oil. ¹H NMR (400 MHz, CDCl₃) δ=7.32-7.17 (m, 5H), 4.61 (d, J=11.6 Hz, 1H), 4.37 (d, J=11.6 Hz, 1H), 3.99 (m, 1H), 3.67 (s, 3H), 1.36 (d, J=6.8 Hz, 3H).

Step 2. To the mixture of methyl (2R)-2-benzyloxypropanoate (8.00 g, 41.0 mmol, 1.0 eq) in 2-MeTHF (100 mL) was added LAH (2.30 g, 62.0 mmol, 1.5 eq) slowly at 0° C. The mixture was stirred at 20° C. for 2 hours. On completion, the mixture was quenched slowly with water (2.3 mL) at 0° C., and then 15% NaOH aqueous solution (2.3 mL), and water (7.0 mL). Upon filtration, the filtrate was concentrated in vacuum and purified by silica gel column (Petroleum ether: EtOAc, from 100:0 to 100:40) to afford (2R)-2-benzyloxypropan-1-ol (7.00 g, 34.0 mmol, 81.79% yield) as colorless oil. ¹H NMR (400 MHz, CDCl₃) δ=7.41-7.29 (m, 5H), 4.67 (d, J=11.6 Hz, 1H), 4.52 (d, J=11.6 Hz, 1H), 3.74-3.67 (m, 1H), 3.66-3.60 (m, 1H), 3.58-3.51 (m, 1H), 1.21 (d, J=6.0 Hz, 3H).

Step 3. To the mixture of (2R)-2-benzyloxypropan-1-ol (7.00 g, 42 mmol, 1.0 eq.) and 2-chloro-N-methyl-acetamide (6.80 g, 63.0 mmol, 1.5 eq.) in t-BuOH (100 mL), t-BuOK (14.2 g, 126 mmol, 3.0 eq.) was added. The mixture was stirred at 25° C. for 16 hours. On completion, the mixture was diluted with EtOAc (80 mL), washed with water (30 mL), sat. NH₄Cl (30 mL), and brine (30 mL). The organic layer was dried over sodium sulfate, concentrated in vacuum and purified by silica gel column (DCM: MeOH, from 100:0 to 100:2) to afford 2-[(2R)-2-benzyloxypropoxy]-N-methyl-acetamide (4.50 g, 17.0 mmol, 40.5% yield) as colorless oil.

¹H NMR (400 MHz, CDCl₃) δ=7.32-7.19 (m, 5H), 7.02 (s, 1H), 4.59 (d, J=11.2 Hz, 1H), 4.39 (d, J=11.2 Hz, 1H), 3.92 (d, J=16.0 Hz, 1H), 3.83 (d, J=16.0 Hz, 1H), 3.70 (t, J=6.4, 3.2 Hz, 1H), 3.50 (dd, J=10.0, 3.2, 1H), 3.36 (dd, J=10.4, 6.8 Hz, 1H), 2.49 (d, J=4.8 Hz, 3H), 1.14 (d, J=6.4 Hz, 3H); LCMS: m/z 238.4 (M+1)⁺.

Step 4. To the mixture of 2-[(2R)-2-benzyloxypropoxy]-N-methyl-acetamide (4.00 g, 16.7 mmol, 1.0 eq.) in 2-MeTHF (100 mL), LAH (959 mg, 25.3 mmol, 1.5 eq.) was added slowly at 0° C. The mixture was stirred at 60° C. for 2 hours. On completion, to the mixture was added water (1 mL) slowly followed by 15% acqueous NaOH (1 mL) and water (3 mL) at 0° C. The mixture was filtered, and the filtrate was concentrated in vacuum to afford 2-[(2R)-2-benzyloxypropoxy]-N-methyl-ethanamine (4.00 g, 11.6 mmol, 69.1% yield).

Step 5. To the mixture of 2-[(2R)-2-benzyloxypropoxy]-N-methyl-ethanamine (3.77 g, 16.9 mmol, 1.0 eq.), DMAP (206 mg, 1.69 mmol, 0.1 eq.), (Boc)₂O(4.42 g, 20.3 mmol, 1.2 eq.) and TEA (2.56 g, 25.3 mmol, 1.5 eq.) in DCM (50 mL), was stirred at 20° C. for 16 hours. The mixture was concentrated in vacuum to afford crude, which was purified by silica gel column (Petroleum ether: EtOAc, from 100:0 to 100:10) to afford tert-butyl N-[2-[(2R)-2-benzyloxypropoxy]ethyl]-N-methyl-carbamate (4.00 g, 10.51 mmol, 62.28% yield) as colorless oil. LCMS: m/z 234.3 (M+1)⁺.

Step 6. To the mixture of tert-butyl N-[2-[(2R)-2-benzyloxypropoxy]ethyl]-N-methyl-carbamate (3.80 g, 11.7 mmol, 1.0 eq.) in MeOH (40 mL) was added Pd(OH)₂ (825 mg, 1.17 mmol, 20% purity, 0.1 eq) under nitrogen atmosphere. The mixture was stirred at 25° C. under 50 Psi H₂ for 16 hours. On completion, the mixture was filtered, and the filtrate was concentrated in vacuum and purified by silica gel column (Petroleum ether: EtOAc, from 100:0 to 100:30) to afford tert-butyl N-[2-[(2R)-2-hydroxypropoxy]ethyl]-N-methyl-carbamate (M5, 2.10 g, 9.00 mmol, 76.6% yield) as colorless oil. ¹H NMR (400 MHz, DMSO-d₆) δ=4.53 (d, J=4.0 Hz, 1H), 3.70 (t, J=5.6 Hz, 1H), 3.52-3.43 (m, 2H), 3.31-3.25 (m, 3H), 3.21-3.14 (m, 1H), 2.80 (d, J=7.2 Hz, 3H), 1.38 (s, 9H), 1.02 (d, J=6.4 Hz, 3H).

### Preparation of Methyl (2R)-3-[tert-butoxycarbonyl(methyl)amino]-2-(2-hydroxyethoxy) propanoate (M6)

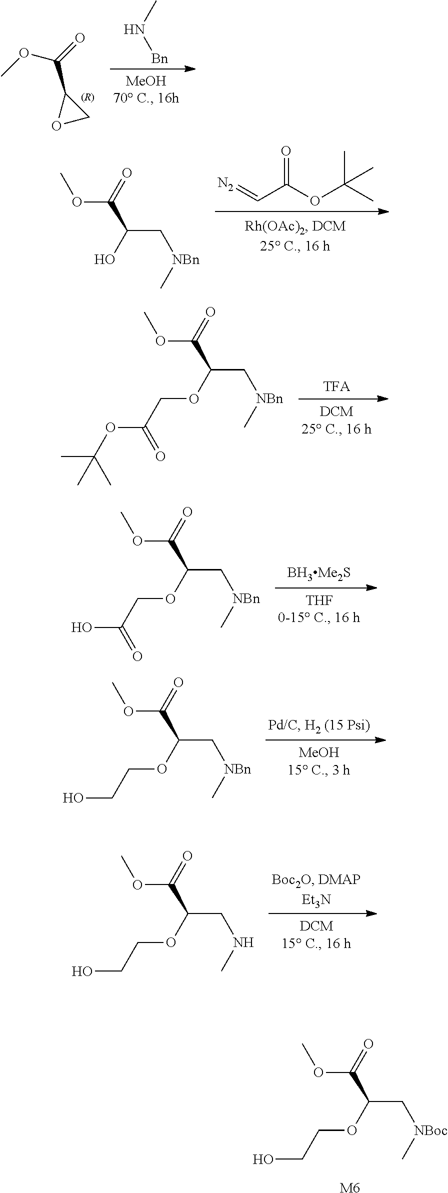

Step 1. The solution of methyl (2R)-oxirane-2-carboxylate (7.00 g, 68.4 mmol, 1 eq.) and N-methyl-1-phenyl-methanamine (8.48 g, 69.9 mmol, 2.26 mL, 1.02 eq.) in MeOH (25 mL) was stirred at 70° C. for 16 hours. LCMS showed desired MS in main peak. The mixture was concentrated in vacuum and the residue was purified by flash chromatography (220 g silica gel column, EtOAc in PE from 0% to 100%) to give Methyl (2R)-3-[benzyl(methyl)amino]-2-hydroxy-propanoate (15.3 g, 68.5 mmol, 99.9% yield) as brown oil. ¹H NMR (400 MHz, CDCl₃) δ=7.40-7.19 (m, 5H), 4.27 (t, J=6.0 Hz, 1H), 3.74 (s, 3H), 3.65 (d, J=13.2 Hz, 1H), 3.52 (d, J=13.2 Hz, 1H), 2.78 (d, J=5.6 Hz, 2H), 2.25 (s, 3H); LC-MS: m/z 224.1 (M+1)⁺.

Step 2. To a solution of methyl (2R)-3-[benzyl(methyl)amino]-2-hydroxy-propanoate (19.0 g, 85.1 mmol, 1 eq.) and Rh(OAc)₂ (940 mg, 4.25 mmol, 0.05 eq.) in DCM (200 mL) was added a solution of tert-butyl 2-diazoacetate (24.2 g, 170 mmol, 2 eq.) in DCM (50 mL) dropwise, the mixture was stirred at 25° C. for 16 hours. The mixture was concentrated in vacuum and the residue was purified by flash chromatography (330 g silica gel column, EtOAc in PE from 0% to 100%) to give Methyl (2R)-3-[benzyl(methyl)amino]-2-(2-tert-butoxy-2-oxo-ethoxy) propanoate (9.80 g, 29.0 mmol, 34.1% yield) as brown oil. ¹H NMR (400 MHz, CDCl₃) δ=7.32-7.23 (m, 5H), 4.28 (t, J=5.2 Hz, 1H), 4.20 (d, J=16.4 Hz, 1H), 3.95 (d, J=16.4 Hz, 1H), 3.75 (s, 3H), 3.66 (d, J=13.2 Hz, 1H), 3.58 (d, J=13.2 Hz, 1H), 2.90-2.88 (m, 2H), 2.30 (s, 3H), 1.49 (s, 9H); LC-MS: m/z 338.2 (M+1)⁺.

Step 3. To a solution of methyl (2R)-3-[benzyl(methyl)amino]-2-(2-tert-butoxy-2-oxo-ethoxy) propanoate (9.80 g, 29.0 mmol, 1 eq.) in DCM (50 mL) was added TFA (77.0 g, 675 mmol, 50 mL, 23.2 eq.). The mixture was stirred at 25° C. for 16 hours. The mixture was concentrated in vacuum and the residue was purified by combi flash (120 g silica gel column, MeOH in DCM from 0% to 30%) to give 2-[(1R)-1-[[benzyl(methyl)amino]methyl]-2-methoxy-2-oxo-ethoxy]acetic acid (8.30 g) as brown oil. ¹H NMR (400 MHz, CDCl₃) δ=7.57-7.55 (m, 2H), 7.49-7.47 (m, 3H), 4.28 (t, J=5.2 Hz, 1H), 4.20 (d, J=16.4 Hz, 1H), 3.95 (d, J=16.4 Hz, 1H), 3.75 (s, 3H), 3.66 (d, J=13.2 Hz, 1H), 3.58 (d, J=13.2 Hz, 1H), 2.90-2.88 (m, 2H), 2.30 (s, 3H), 1.49 (s, 9H); LC-MS: m/z 282.4 (M+1)⁺.

Step 4. To a solution of 2-[(1R)-1-[[benzyl(methyl)amino]methyl]-2-methoxy-2-oxo-ethoxy]acetic acid (8.30 g, 29.5 mmol, 1 eq.) in THF (80 mL) was added BH3-Me2S (10 M, 8.85 mL, 3 eq.) at 0° C. The mixture was stirred at 15° C. for 16 hours. The mixture was quenched by MeOH (3 mL) and concentrated in vacuum. The residue was purified by combi flash (80 g silica gel column, EtOAc in PE from 0% to 100%, MeOH in EtOAc from 0% to 100%) to give methyl (2R)-3-[benzyl(methyl)amino]-2-(2-hydroxyethoxy)propanoate (4.60 g, 10.8 mmol, 36.7% yield) as brown oil. LC-MS: m/z 238.1 (M+1)⁺.

Step 5. To a mixture of methyl (2R)-3-[benzyl(methyl)amino]-2-(2-hydroxyethoxy) propanoate (2.60 g, 9.73 mmol, 1 eq.) in MeOH (30 mL) was added Pd/C (400 mg, 10% purity). The mixture was stirred at 15° C. under H₂ (15 Psi) for 3 hours. The mixture was filtered and the filtrate was concentrated in vacuum to give Methyl (2R)-2-(2-hydroxyethoxy)-3-(methylamino) propanoate (1.3 g) as colorless oil. LC-MS: m/z 178.1 (M+1)⁺.

Step 6. To a solution of methyl (2R)-2-(2-hydroxyethoxy)-3-(methylamino) propanoate (2.70 g, 15.2 mmol, 1 eq.) and Et₃N (3.08 g, 30.5 mmol, 4.24 mL, 2 eq.) in DCM (30 mL) was added DMAP (186 mg, 1.52 mmol, 0.1 eq.) and Boc₂O(4.99 g, 22.8 mmol, 5.25 mL, 1.5 eq.). The mixture was stirred at 15° C. for 16 hours. The mixture was concentrated in vacuum and the residue was purified by combi flash (20 g silica gel column, EtOAc in PE from 0% to 100%) to give Methyl (2R)-3-[tert-butoxycarbonyl(methyl)amino]-2-(2-hydroxyethoxy) propanoate (1.15 g, 4.15 mmol, 27.22% yield) as colorless oil. LC-MS: m/z 278.1 (M+1)⁺.

### Preparation of tert-butyl N-[2-(2-hydroxyethylsulfanyl)ethyl]-N-methyl-carbamate (M8)

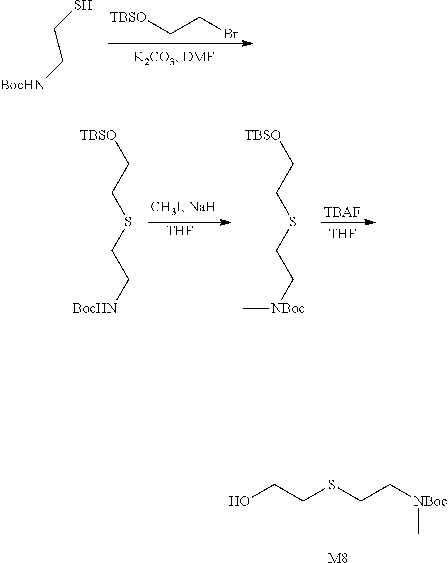

Step 1. To a solution of tert-butyl N-(2-sulfanylethyl)carbamate (3.7 g, 20.9 mmol, 1 eq) and 2-bromoethoxy-tert-butyl-dimethyl-silane (5.2 g, 21.7 mmol, 1.04 eq) in DMF (10 mL) was added K₂CO₃ (5.77 g, 41.75 mmol, 2 eq). The mixture was stirred at 25° C. for 10 hours. On completion, the mixture was quenched with water (5 mL) and extracted with EtOAc (10 mL×3). The combined organic phase was dried over anhydrous sodium sulfate, filtered and concentrated. The residue was purified by column chromatography (SiO₂, Petroleum ether/Ethyl acetate=30/1 to 20/1) to give tert-butyl N-[2-[2-[tert-butyl(dimethyl)silyl]oxyethylsulfanyl]ethyl]carbamate (5.5 g, 16.39 mmol, 78.5% yield) as a light yellow solid product. ¹H NMR (400 MHz, DMSO-d₆) δ=6.84 (t, J=5.6 Hz, 1H), 3.66 (t, J=6.8 Hz, 2H), 3.06-2.97 (m, 2H), 2.55 (t, J=6.8 Hz, 2H), 2.51-2.47 (m, 2H), 1.32 (s, 9H), 0.82 (s, 9H), 0.00 (s, 6H); LC-MS: m/z 236.1 (M-99)⁺.

Step 2. To a mixture of tert-butyl N-[2-[2-[tert-butyl(dimethyl)silyl]oxyethylsulfanyl]ethyl]carba mate (5.5 g, 16.4 mmol, 1 eq) in THF (90 mL) at 0° C. was added NaH (983 mg, 24.6 mmol, 60% purity, 1.5 eq). The reaction was stirred under N₂ at 0° C. for 15 minutes followed by addition CH₃I (3.49 g, 24.6 mmol, 1.5 eq) dropwise. The reaction was stirred under N₂ at 25° C. for 6 hours. On completion, the mixture was quenched with water (10 mL) and then diluted with H₂O (90 mL) and extracted with EtOAc 90 mL (30 mL×3). The combined organic phase was dried over anhydrous sodium sulfate, filtered and concentrated. The residue was purified by column chromatography (SiO₂, Petroleum ether/Ethyl acetate=30/1 to 10/1) to give tert-butyl N-[2-[2-[tert-butyl(dimethyl)silyl]oxyethylsulfanyl]ethyl]-N-methyl-carbamate (4 g, 11.1 mmol, 67.7% yield, 97% purity) was obtained as a light yellow solid product. ¹H NMR (400 MHz, DMSO-d₆) δ=3.67 (t, J=6.8 Hz, 2H), 3.27-3.23 (m, 2H), 2.72 (s, 3H), 2.62-2.54 (m, 4H), 1.34 (s, 9H), 0.81 (s, 9H), 0.00 (s, 6H).

Step 3. To a solution of tert-butyl N-[2-[2-[tert-butyl(dimethyl)silyl]oxyethylsulfanyl]ethyl]-N-methyl-carbamate (4 g, 11.4 mmol, 1 eq) in THF (160 mL) was added TBAF (1 M, 34.3 mL, 3 eq). The mixture was stirred at 25° C. for 2 hours. On completion, the mixture was quenched with saturated ammonium chloride aqueous solution (100 mL) at 0° C., and then diluted with H₂O (50 mL) and extracted with EtOAc (100 mL×3). The combined organic phase was dried over anhydrous sodium sulfate, filtered and concentrated. The residue was purified by column chromatography (SiO₂, Petroleum ether/Ethyl acetate=I/O to 2/1) to give tert-butyl N-[2-(2-hydroxyethylsulfanyl)ethyl]-N-methyl-carbamate (M8, 2.6 g, 10.5 mmol, 91.7% yield) as a light yellow solid product. 1H NMR (400 MHz, DMSO-d₆) δ=4.82 (t, J=5.2 Hz, 1H), 3.62-3.54 (m, 2H), 3.34-3.32 (m, 2H), 2.82 (s, 3H), 2.66 (t, J=7.2 Hz, 2H), 2.62 (t, J=7.2 Hz, 2H), 1.43 (s, 9H).

### Preparation of tert-butyl N-[2-[benzyloxycarbonyl (2-hydroxyethyl)amino]ethyl]-N-methyl-carbamate (M9)

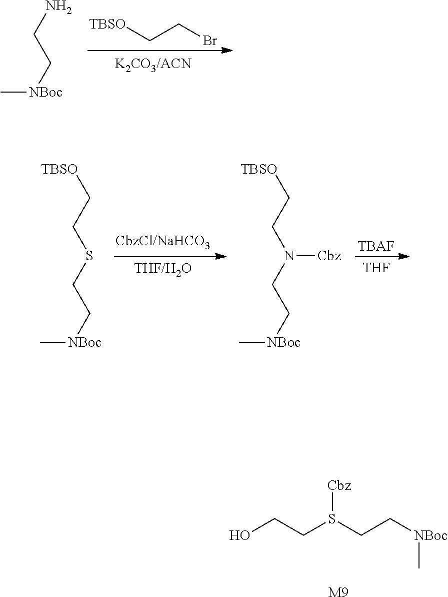

Step 1. To a solution of tert-butyl N-(2-aminoethyl)-N-methyl-carbamate (10.0 g, 57.3 mmol, 10.2 mL, 1 eq) and 2-bromoethoxy-tert-butyl-dimethyl-silane (10.9 g, 45.9 mmol, 0.8 eq) in ACN (150 mL) was added K₂CO₃ (23.8 g, 172 mmol, 3 eq). The mixture was stirred at 80° C. for 16 hr. On completion, the mixture was quenched with water (200 mL), and extracted with EtOAc (3×150 mL). Combined organic layer was washed with brine (150 mL), dried over sodium sulfate, concentrated in vacuum. The residue was purified by flash silica gel chromatography (120 g silica gel column, DCM in MeOH from 0% to 100%) to give tert-butyl N-[2-[2-[tert-butyl (dimethyl) silyl]oxyethylamino]ethyl]-N-methyl-carbamate (7.50 g, 18.0 mmol, 31.4% yield) as colorless gum. LC-MS: m/z 333.8 (M+1)⁺.

Step 2. To a solution of tert-butyl N-[2-[2-[tert-butyl (dimethyl) silyl]oxyethylamino]ethyl]-N-methyl-carbamate (2.70 g, 8.12 mmol, 1 eq) in THF (80 mL) and H₂O (20 mL) was added CbzCl (1.80 g, 10.5 mmol, 1.50 mL, 1.3 eq) and NaHCO₃ (2.05 g, 24.3 mmol, 947 uL, 3 eq). The mixture was stirred at 25° C. for 16 hr. On completion, the mixture was diluted with water (100 mL) and extracted by EtOAc (3*80 mL). The combined organic phase was dried over Na₂SO₄, filtered and concentrated. The residue was purified by flash silica gel chromatography (40.0 g silica gel column, PE in EA from 0% to100%) to give tert-butyl N-[2-[benzyloxycarbonyl-[2-[tert-butyl (dimethyl) silyl]oxyethyl]amino]ethyl]-N-methyl-carbamate (3.80 g, 7.33 mmol, 90.2% yield) as colorless gum. ¹H NMR (400 MHz, DMSO-d₆) δ=7.34-7.30 (m, 5H), 5.05 (s, 2H), 3.72-3.60 (m, 2H), 3.38 (s, 2H), 3.38-3.31 (m, 4H), 2.80-2.66 (m, 3H), 1.35 (s, 9H), 0.83 (d, J=10.8 Hz, 9H), 0.07-0.09 (m, 6H); LC-MS: m/z 367.6 (M-99)⁺.

Step 3. To a solution of tert-butyl N-[2-[benzyloxycarbonyl-[2-[tert-butyl(dimethyl)silyl]oxyethyl]amino]ethyl]-N-methyl-carbamate (1.00 g, 2.14 mmol, 1 eq) in THF (20 mL) was added tetrabutylammonium fluoride trihydrate (1 M, 4.29 mL, 2 eq). The mixture was stirred at 25° C. for 2 hr. On completion, the mixture was quenched with NH₄Cl (8 mL), and extracted with EtOAc (3×30 mL). Combined organic layer was washed with brine (50 mL), dried over sodium sulfate, and concentrated in vacuum. The residue was purified by flash silica gel chromatography (12 g silica gel column, DCM in MeOH from 0% to 100%) to give tert-butyl N-[2-[benzyloxycarbonyl (2-hydroxyethyl)amino]ethyl]-N-methyl-carbamate (M9, 500 mg, 1.21 mmol, 56.2% yield) as colorless gum. ¹H NMR (400 MHz, DMSO-d₆) δ=7.31-7.05 (m, 5H), 4.86 (s, 2H), 4.59-4.48 (m, 1H), 3.29 (s, 2H), 3.18 (d, J=5.3 Hz, 2H), 3.15-3.03 (m, 4H), 2.57 (s, 2H), 1.24-1.10 (m, 9H); LCMS: m/z 253.0 (M-99)⁺.

### Preparation of tert-butyl N-[2-[2-(6-chloro-2-oxo-indolin-5-yl)oxyethyl-methyl-amino]ethyl]-N-methyl-carbamate (M10)

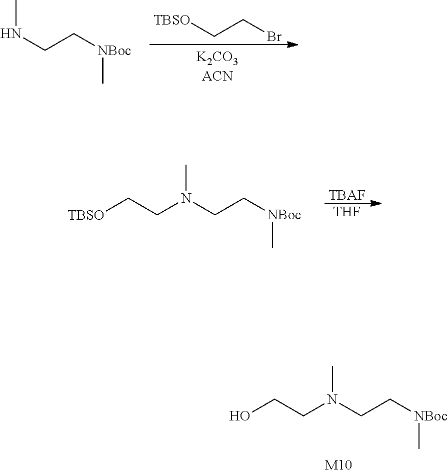

Step 1. To a solution of tert-butyl N-methyl-N-[2-(methylamino)ethyl]carbamate (15.0 g, 79.6 mmol, 1 eq) and 2-bromoethoxy-tert-butyl-dimethyl-silane (19.0 g, 79.6 mmol, 1 eq) in ACN (300 mL) was added K₂CO₃ (11.0 g, 79.6 mmol, 1 eq). The mixture was stirred at 80° C. for 16 hr. On completion, the mixture was quenched with water (200 mL), and extracted with EtOAc (3×200 mL). Combined organic layer was washed with brine (150 mL), dried over sodium sulfate, and concentrated in vacuum. The residue was purified by flash silica gel chromatography (220 g silica gel column, DCM in MeOH from 0% to 100%) to give tert-butyl N-[2-[2-[tert-butyl (dimethyl) silyl]oxyethyl-methyl-amino]ethyl]-N-methyl-carbamate (16 g, 39.2 mmol, 49.2% yield) as colorless gum. 1H NMR (400 MHz, DMSO-d₆) δ=3.62 (t, J=6.4 Hz, 2H), 3.20 (t, J=6.8 Hz, 2H), 2.76 (s, 3H), 2.46 (s, 4H), 2.22 (s, 3H), 1.38 (s, 9H), 0.85 (s, 9H), 0.03 (s, 6H).

Step 2. To a solution of tert-butyl N-[2-[2-[tert-butyl(dimethyl)silyl]oxyethyl-methyl-amino]ethyl]-N-methyl-carbamate (15.0 g, 43.2 mmol, 1 eq) in THF (400 mL) was added tetrabutylammonium fluoride trihydrate (1 M, 86.5 mL, 2 eq). The mixture was stirred at 25° C. for 16 hr. On completion, the mixture was diluted with water (200 mL) and extracted by DCM (3*180 mL). The combined organic phase was dried over Na₂SO₄, filtered and concentrated. The residue was purified by flash silica gel chromatography (180 g silica gel column, DCM in MeOH from 0% to 100%) to give tert-butyl N-[2-[2-hydroxyethyl(methyl)amino]ethyl]-N-methyl-carbamate (M10, 9 g, 34.8 mmol, 80.5% yield) as colorless gum. ¹H NMR (400 MHz, DMSO-d₆) δ=4.30 (s, 1H), 3.48-3.40 (m, 2H), 3.20 (t, J=7.0 Hz, 2H), 2.76 (s, 3H), 2.43 (q, J=6.7 Hz, 4H), 2.20 (s, 3H), 1.38 (s, 9H).

### Preparation of tert-butyl N-methyl-N-[2-[2-(2-oxoindolin-5-yl)oxyethoxy]ethyl]carbamate (M-1) according to General Method M

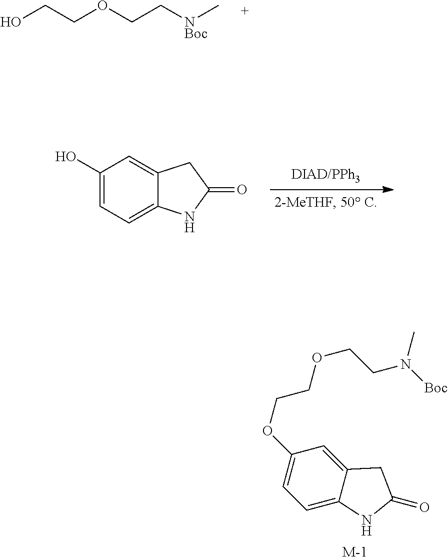

To a solution of 5-hydroxyindolin-2-one (400 mg, 2.68 mmol, 1 eq), PPh₃ (1.55 g, 5.90 mmol, 2.2 eq) and tert-butyl N-[2-(2-hydroxyethoxy)ethyl]-N-methyl-carbamate (1.18 g, 5.36 mmol, 2.0 eq) in 2-MeTHF (20 mL) was DIAD (1.19 g, 5.90 mmol, 1.15 mL, 2.2 eq) in an ice-bath. The mixture was stirred at 50° C. for 16 h, quenched with MeOH (1 mL), and concentrated in vacuum. The residue was purified by silica gel column (DCM: MeOH=100: 0-100:3) to afford tert-butyl N-methyl-N-[2-[2-(2-oxoindolin-5-yl)oxyethoxy]ethyl]carbamate (M-1, 400 mg, 0.719 mmol, 26.8% yield) as light brown gum. LCMS: m/z 251.3 (M+1)⁺.

M-2-M-10 were prepared following similar procedures as M-1.

| Comp. | | | |
|---|---|---|---|
| # | Structure | ¹NMR (400 MHz, DMSO-d₆) δ (ppm) | MS m/z |
| M-1 | 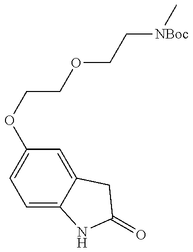 [SMILES: CN(CCOCCOc1ccc2c(c1)CC(=O)N2)C(=O)OC(C)(C)C] | | 251.3 (M − Boc)⁺ |
| M-2 | 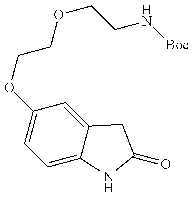 [SMILES: CC(C)(C)OC(=O)NCCOCCOc1ccc2c(c1)CC(=O)N2] | 10.17 (s, 1H), 6.86 (s, 1H), 6.74 (s, 1H), 6.72 (d, J = 8.0 Hz, 1H), 6.69 (d, J = 8.0 Hz, 1H), 3.99 (t, J = 4.4 Hz, 2H), 3.67 (t, J = 4.4 Hz, 2H), 3.43-3.41 (m, 4H), 3.08 (dd, J = 12.4, 6.0 Hz, 3H), 1.37 (s, 9H) | 237.0 (M − Boc)⁺ |
| M-3 | 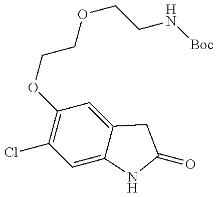 [SMILES: CC(C)(C)OC(=O)NCCOCCOc1cc2c(cc1Cl)NC(=O)C2] | 6.94 (s, 1H), 6.91 (s, 1H), 4.13 (t, J = 4.8 Hz, 2H), 3.84 (t, J = 4.8 Hz, 2H), 3.64 (t, J = 5.2 Hz, 2H), 3.53-3.48 (m, 2H), 3.40-3.30 (m, 2H), 1.45 (s, 9H) | 271 (M − Boc)⁺ |
| M-4 | 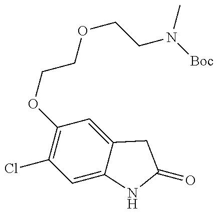 [SMILES: CN(CCOCCOc1cc2c(cc1Cl)NC(=O)C2)C(=O)OC(C)(C)C] | | 285.1 (M − Boc)⁺ |
| M-5 | 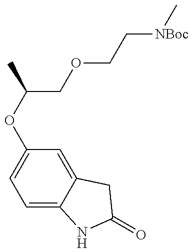 [SMILES: C[C@@H](COCCN(C)C(=O)OC(C)(C)C)Oc1ccc2c(c1)CC(=O)N2] | 10.17 (s, 1H), 6.86 (s, 1H), 6.74 (d, J = 8.4 Hz, 1H), 6.67 (d, J = 8.4 Hz, 1H), 4.40 (t, J = 5.2 Hz, 1H), 3.56-3.44 (m, 4H), 3.41 (s, 2H), 3.31-3.27 (m, 2H), 2.79 (s, 3H), 1.37 (s, 9H), 1.17 (d, J = 6.4 Hz, 3H) | 387.4 (M + 23)⁺ |
| M-6 | 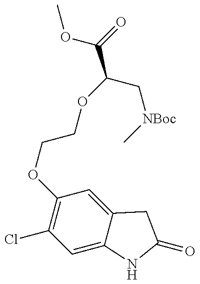 [SMILES: COC(=O)[C@@H](CN(C)C(=O)OC(C)(C)C)OCCOc1cc2c(cc1Cl)NC(=O)C2] | ¹H NMR (400 MHz, CDCl₃) δ = 8.01 (s, 1H), 6.93 (s, 1H), 6.90 (s, 1H), 4.33 (s, 1H), 4.14 (d, J = 3.2 Hz, 2H), 4.06- 3.95 (m, 1H), 3.89-3.77 (m, 1H), 3.74 (s, 3H), 3.71 (d, J = 8.0 Hz, 1H), 2.94 (s, 3H), 1.50 (s, 9H) | 343.0 (M − Boc)⁺. |
| M-7 | 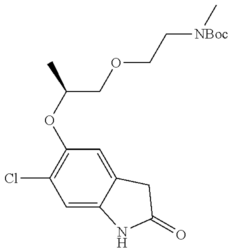 [SMILES: C[C@@H](COCCN(C)C(=O)OC(C)(C)C)Oc1cc2c(cc1Cl)NC(=O)C2] | | 299.1 (M − Boc)⁺ |
| M-8 | 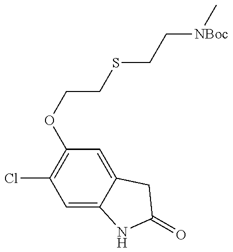 [SMILES: CN(CCSCCOc1cc2c(cc1Cl)NC(=O)C2)C(=O)OC(C)(C)C] | 10.28 (s, 1H), 7.15 (s, 1H), 6.82 (s, 1H), 4.18-4.11 (m, 2H), 3.46 (s, 2H), 3.35 (s, 2H), 2.92-2.86 (m, 2H), 2.78-2.75 (m, 5H), 1.38 (s, 9H) | 301 (M − Boc)⁺ |
| M-9 | 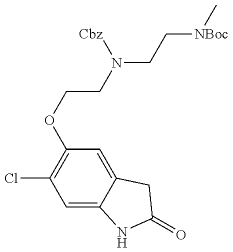 [SMILES: CN(CCN(CCOc1cc2c(cc1Cl)NC(=O)C2)C(=O)OCc1ccccc1)C(=O)OC(C)(C)C] | | 418.2 (M − Boc)⁺ |
| M-10 | 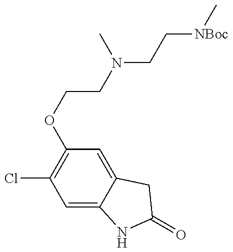 [SMILES: CN(CCOc1cc2c(cc1Cl)NC(=O)C2)CCN(C)C(=O)OC(C)(C)C] | 10.26 (s, 1H), 7.12 (s, 1H), 6.81 (s, 1H), 4.34 (t, J = 5.0 Hz, 2H), 4.03 (t, J = 5.8 Hz, 2H), 3.45 (s, 3H), 3.44 (d, J = 2.0 Hz, 2H), 3.26-3.21 (m, 2H), 2.81- 2.77 (m, 2H), 2.30 (s, 3H), 1.37 (s, 9H) | 398.1 (M + 1)⁺ |

### Preparation of tert-butyl N-methyl-N-[2-[2-(2-oxoindolin-5-yl)sulfanylethoxy]ethyl]carbamate (M-1s)

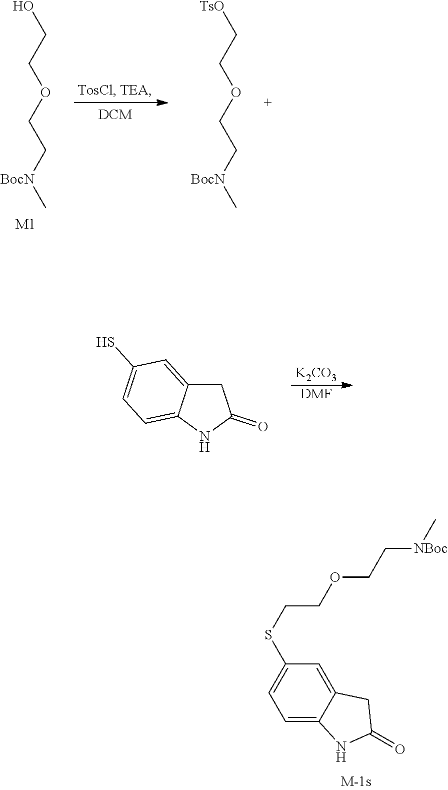

Step 1. To a mixture of tert-butyl N-[2-(2-hydroxyethoxy)ethyl]-N-methyl-carbamate (1.00 g, 4.56 mmol, 1 eq) and TEA (1.38 g, 13.7 mmol, 3 eq) in DCM (10 mL) was added TosCl (1.30 g, 6.84 mmol, 1.5 eq) at 0° C. The mixture was stirred at 25° C. for 12 hours and partitioned with H₂O (5 mL). The organic phase was separated, washed with H₂O (5 mL*2), dried over Na₂SO₄, filtered and concentrated under reduced pressure. The residue was purified by flash silica gel chromatography to afford 2-[2-[tert-butoxycarbonyl(methyl)amino]ethoxy]ethyl 4-methylbenzenesulfonate (1.70 g, 4.28 mmol, 93.8% yield) as a light yellow oil. LCMS: m/z 275 (M-Boc)⁺.

Step 2. To a solution of 5-sulfanylindolin-2-one (330 mg, 2.00 mmol, 1 eq) in DMF (5 mL) was added K₂CO₃ (303 mg, 2.20 mmol, 1.1 eq) and 2-[2-[tert-butoxycarbonyl(methyl)amino]ethoxy]ethyl 4-methylbenzenesulfonate (597 mg, 1.60 mmol, 0.8 eq). The mixture was stirred at 25° C. for 2 hours under N₂ atmosphere and partitioned between H₂O (10 mL) and EtOAc (10 mL). The organic phase was separated, washed with salt water (5 mL*3), dried over Na₂SO₄, filtered and concentrated under reduced pressure. The residue was purified by flash silica gel chromatography to afford tert-butyl N-methyl-N-[2-[2-(2-oxoindolin-5-yl)sulfanylethoxy]ethyl]carbamate (M-1s, 450 mg, 1.06 mmol, 52.8% yield) as a yellow oil. LCMS: m/z 267.4 (M-Boc)⁺.

### Preparation of tert-butyl N-[2-[2-(6-chloro-2-oxo-indolin-5-yl)sulfanylethoxy]ethyl]-N-methyl-carbamate (M-2s)

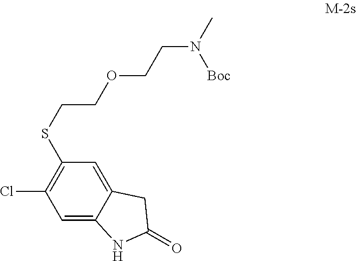 [SMILES: CN(CCOCCSc1cc2c(cc1Cl)NC(=O)C2)C(=O)OC(C)(C)C]

M-2s was prepared using similar procedures as M-1s using 6-chloro-5-sulfanylindolin-2-one. ¹H NMR (400 MHz, DMSO-d₆) δ=10.50 (s, 1H), 7.38 (s, 1H), 6.88 (s, 1H), 3.56 (t, J=6.4 Hz, 2H), 3.48 (s, 3H), 3.46 (s, 1H), 3.31 (s, 1H), 3.29-3.26 (m, 2H), 3.06 (t, J=6.4 Hz, 2H), 2.78 (d, J=9.2 Hz, 2H), 1.37 (s, 9H). LCMS: m/z 301.0 (M-Boc)⁺.

### Preparation of 2-formyl-N,5-dimethyl-N-[2-[2-[5-(2-oxoindolin-5-yl)pyrazol-1-yl]ethoxy]ethyl]-1H-pyrrole-3-carboxamide (N-1) according to General Method N

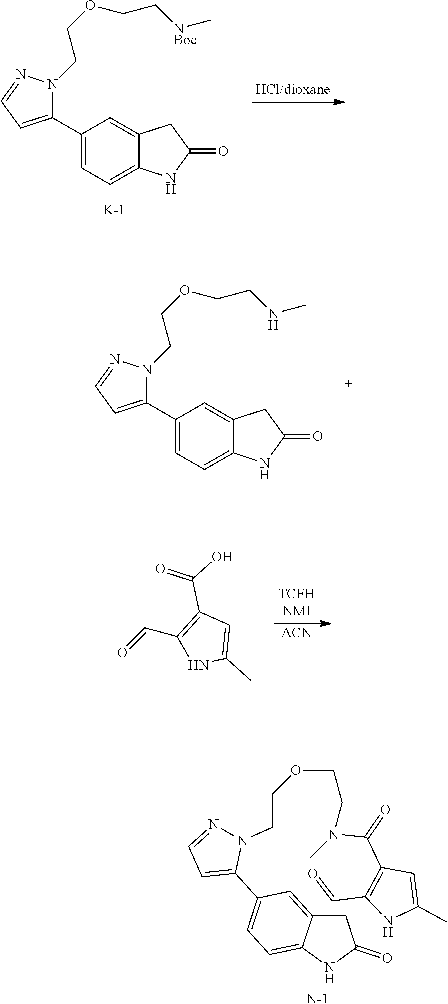

Step 1. To a solution of tert-butyl N-methyl-N-[2-[2-[5-(2-oxoindolin-5-yl)pyrazol-1-yl]ethoxy]ethyl]carbamate (150 mg, 374 umol, 1 eq) in DCM (5 mL) was added HCl/dioxane (4 M, 0.94 mL, 10 eq) and the resulting mixture was stirred at 25° C. for 1 h. The reaction mixture was concentrated in vacuum to give 5-[2-[2-[2-(methylamino) ethoxy]ethyl]pyrazol-3-yl]indolin-2-one HCl salt (123 mg, 0.34 mmol, 90% yield) as a white solid. LCMS: m/z 301.3 (M+1)⁺.

Step 2. To a solution of 5-[2-[2-[2-(methylamino)ethoxy]ethyl]pyrazol-3-yl]indolin-2-one HCl salt (113 mg, 0.34 mmol), 2-formyl-5-methyl-1H-pyrrole-3-carboxylic acid (51.4 mg, 0.34 mmol, 1 eq) in acetonitrile (1 mL) were added 1-methylimidazole (82.6 mg, 1.01 mmol, 3 eq) and [chloro(dimethylamino)methylene]-dimethyl-ammonium hexafluorophosphate (141.2 mg, 0.50 mmol, 1.5 eq) and the mixture stirred at 25° C. for 0.5 h. The reaction mixture was concentrated in vacuum and purified by column chromatography on silica gel (DCM: MNH=30:1-M 10:1). The crude product was triturated with MeSH (5 mL) at 25° C. for 10 min and then filtered to give 2-formyl-N,5-dimethyl-N-12-112-15-(2-oxoindolin-5-yl)pyrazol-1-yl]ethoxy]ethyl]1H-pyrrole-3-carboxamide (N-1, 110 mg, 0.21 mmol, 62% yield) as a yellow oil. H NMR (400 MHz, DMO-H), 6) (ppm) 12.06 (s, 1H), 10.50 (s, 1H), 9.43-49.20 (i, 1H), 8.57 (s, 2H), 7.49 (d, J=1.6 Hz, 1H), 6.88 (d, J=8.0 Hz, 1H), 6.25 (d, J=2.0 Hz, 1H), 6.04-5.86 (m, 1H), 4.19 (s, 2H), 3.52-3.48 (s, 5H), 3.44-3.42 (s, 6H), 2.85 (s, 3H). LCMS: m6z 436.3 (M+1)₁.

N-2-N-39 were prepared following similar procedures as N-1, using corresponding intermediates K-2, L-1-L-13, M-1-M-10, M-1s and M-2s with the corresponding pyrrole aldehyde.

| Comp. | | ¹NMR (400 MHz, DMSO-d₆) δ | |
|---|---|---|---|
| # | Structure | (ppm) | MS m/z |
| N-1 | 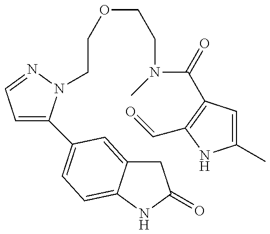 | 8.17 (s, 1H), 7.58 (d, J = 6.8 Hz, 1H), 7.41-7.35 (m, 2H), 6.96 (d, J = 2.4 Hz, 1H), 6.25 (d, J = 1.6 Hz, 1H), 4.27 (t, J = 5.6 Hz, 2H), 3.92 (t, J = 5.6 Hz, 2H), 3.61 (s, 2H), 3.48 (s, 2H), 3.30 (d, J = 5.6 Hz, 2H), 2.78 (s, 3H), 1.42 (s, 9H). | 401.0 (M + 1)⁺ |
| N-2 | 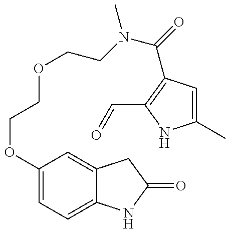 [SMILES: Cc1cc(C(=O)N(C)CCOCCOc2ccc3c(c2)CC(=O)N3)c(C=O)[nH]1] | 9.82 (s, 1H), 9.57 (s, 1H), 8.25-8.02 (m, 1H), 6.85 (s, 1H), 6.75 (s, 2H), 6.10 (d, J = 9.2 Hz, 1H), 4.08-4.05 (m, 2H), 3.89-3.72 (m, 6H), 3.65 (s, 2H), 3.17-3.13 (m, 3H), 2.34 (s, 3H) | 386.3 (M + 1)⁺ |
| N-3 | 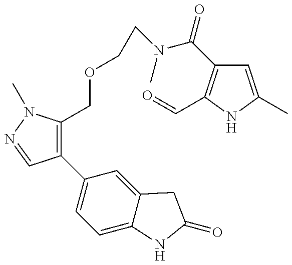 [SMILES: Cc1cc(C(=O)N(C)CCOCc2c(-c3ccc4c(c3)CC(=O)N4)cnn2C)c(C=O)[nH]1] | 12.08 (d, J = 26.8 Hz, 1H), 10.38 (s, 1H), 9.32 (d, J = 45.6 Hz, 1H), 7.54 (s, 1H), 7.19 (d, J = 14.0 Hz, 2H), 6.83 (d, J = 7.6 Hz, 1H), 6.03 (d, J = 19.2 Hz, 1H), 4.53 (d, J = 30.4 Hz, 2H), 3.84-3.77 (m, 3H), 3.74-3.60 (m, 2H), 3.51 (d, J = 32.4 Hz, 2H), 3.47 (s, 2H), 2.95 (d, J = 23.6 Hz, 3H), 2.20 (d, J = 22.4 Hz, 3H) | 436.2 (M + 1)⁺ |
| N-4 | 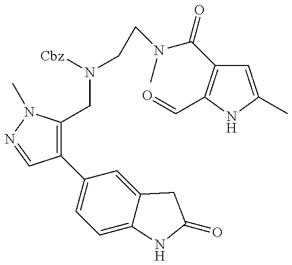 [SMILES: Cc1cc(C(=O)N(C)CCN(Cc2c(-c3ccc4c(c3)CC(=O)N4)cnn2C)C(=O)OCc2ccccc2)c(C=O)[nH]1] | 12.11 (s, 1H), 10.38 (s, 1H), 9.37 (s, 1H), 8.34 (s, 1H), 7.45-7.39 (m, 1H), 7.36-7.25 (m, 5H), 7.24-7.17 (m, 1H), 6.80 (d, J = 6.8 Hz, 1H), 5.95 (s, 1H), 5.04 (s, 1H), 4.97-4.87 (m, 1H), 4.79 (s, 1H), 4.45 (s, 1H), 3.76 (s, 6H), 3.24 (s, 3H), 3.16-3.04 (m, 2H), 2.27-2.18 (m, 2H) | 569.4 (M + 1)⁺ |
| N-5 | 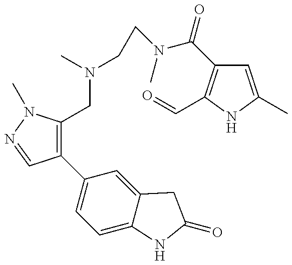 [SMILES: Cc1cc(C(=O)N(C)CCN(C)Cc2c(-c3ccc4c(c3)CC(=O)N4)cnn2C)c(C=O)[nH]1] | | 449.1 (M + 1)⁺ |
| N-6 | 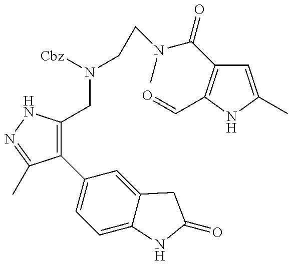 [SMILES: Cc1cc(C(=O)N(C)CCN(Cc2[nH]nc(C)c2-c2ccc3c(c2)CC(=O)N3)C(=O)OCc2ccccc2)c(C=O)[nH]1] | 12.50 (s, 1H), 12.08 (s, 1H), 10.36 (s, 1H), 9.43-9.25 (m, 1H), 7.39-7.13 (m, 5H), 7.12-6.92 (m, 2H), 6.78 (s, 1H), 5.97 (s, 1H), 5.04-4.87 (m, 1H), 4.85-4.75 (m, 1H), 4.50 (s, 1H), 4.17 (s, 1H), 3.41 (s, 3H), 2.90 (s, 2H), 2.79 (s, 2H), 2.25-2.20 (m, 2H), 2.19-2.14 (m, 3H), 2.13-2.12 (m, 2H), 1.99 (s, 3H) | 569.5 (M + 1)⁺ |
| N-7 | 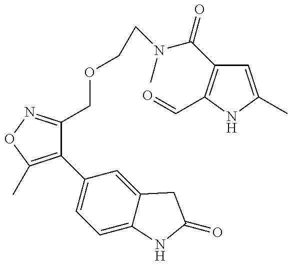 [SMILES: Cc1cc(C(=O)N(C)CCOCc2noc(C)c2-c2ccc3c(c2)CC(=O)N3)c(C=O)[nH]1] | | 437.0 (M + 1)⁺ |
| N-8 | 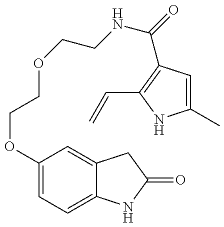 [SMILES: C=Cc1[nH]c(C)cc1C(=O)NCCOCCOc1ccc2c(c1)CC(=O)N2] | | 372.4 (M + 1)⁺ |
| N-9 | 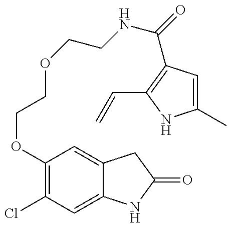 [SMILES: C=Cc1[nH]c(C)cc1C(=O)NCCOCCOc1cc2c(cc1Cl)NC(=O)C2] | | 406.2 (M + 1)⁺ |
| N-10 | 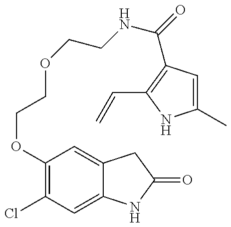 | | 420.1 (M + 1)⁺ |
| N-11 | 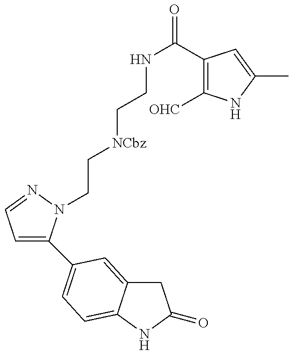 [SMILES: Cc1cc(C(=O)NCCN(CCn2nccc2-c2ccc3c(c2)CC(=O)N3)C(=O)OCc2ccccc2)c(C=O)[nH]1] | | 555.6 (M + 1)⁺ |
| N-12 | 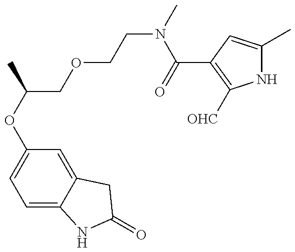 [SMILES: Cc1cc(C(=O)N(C)CCOC[C@H](C)Oc2ccc3c(c2)CC(=O)N3)c(C=O)[nH]1] | | 400.4 (M + 1)⁺ |
| N-13 | 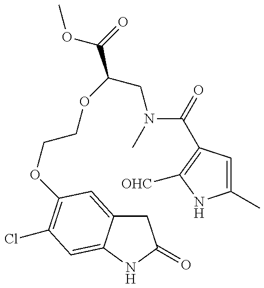 [SMILES: COC(=O)[C@@H](CN(C)C(=O)c1cc(C)[nH]c1C=O)OCCOc1cc2c(cc1Cl)NC(=O)C2] | | 478.1 (M + 1)⁺ |
| N-14 | 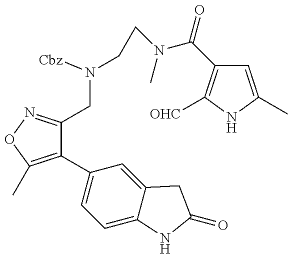 [SMILES: Cc1cc(C(=O)N(C)CCN(Cc2noc(C)c2-c2ccc3c(c2)CC(=O)N3)C(=O)OCc2ccccc2)c(C=O)[nH]1] | 12.09 (s, 1H), 10.46 (s, 1H), 9.41- 9.21 (m, 1H), 7.22-6.95 (m, 8H), 5.98 (s, 1H), 5.05-4.51 (m, 4H), 4.24 (br, 1H), 4.09 (dd, J = 1.6, 2.8 Hz, 1H), 3.74 (s, 1H), 3.54 (s, 3H), 3.41 (s, 3H), 2.94 (s, 2H), 2.83 (br, 2H), 2.33 (s, 3H). | 570.4 (M + 1)⁺ |
| N-15 | 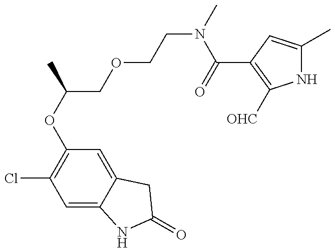 [SMILES: Cc1cc(C(=O)N(C)CCOC[C@H](C)Oc2cc3c(cc2Cl)NC(=O)C3)c(C=O)[nH]1] | | 434.1 (M + 1)⁺ |
| N-16 | 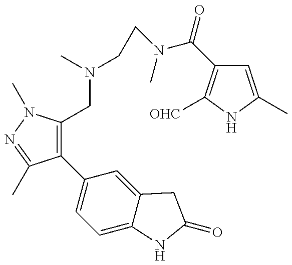 [SMILES: Cc1cc(C(=O)N(C)CCN(C)Cc2c(-c3ccc4c(c3)CC(=O)N4)c(C)nn2C)c(C=O)[nH]1] | | 463.2 (M + 1)⁺ |
| N-17 | 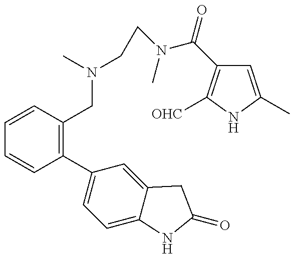 [SMILES: Cc1cc(C(=O)N(C)CCN(C)Cc2ccccc2-c2ccc3c(c2)CC(=O)N3)c(C=O)[nH]1] | | 445.2 (M + 1)⁺ |
| N-18 | 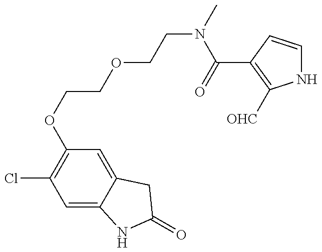 [SMILES: CN(CCOCCOc1cc2c(cc1Cl)NC(=O)C2)C(=O)c1cc[nH]c1C=O] | | 406.3 (M + 1)⁺ |
| N-19 | 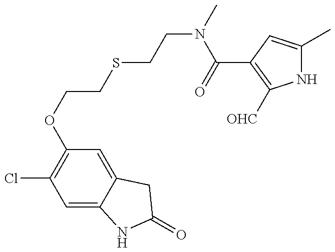 [SMILES: Cc1cc(C(=O)N(C)CCSCCOc2cc3c(cc2Cl)NC(=O)C3)c(C=O)[nH]1] | 12.19 (s, 1H), 10.34 (s, 1H), 9.52- 9.38 (m, 1H), 6.83 (s, 1H), 6.12 (s, 1H), 4.18 (s, 2H), 3.47 (s, 2H), 3.03- 2.82 (m, 7H), 2.69 (s, 3H), 2.23- 2.20 (m, 3H). | 436.0 (M + 1)⁺ |
| N-20 | 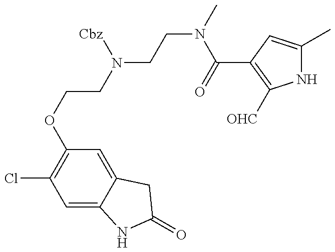 [SMILES: Cc1cc(C(=O)N(C)CCN(CCOc2cc3c(cc2Cl)NC(=O)C3)C(=O)OCc2ccccc2)c(C=O)[nH]1] | | 553.1 (M + 1)⁺ |
| N-21 | 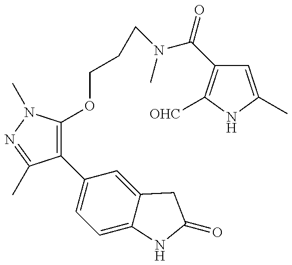 [SMILES: Cc1cc(C(=O)N(C)CCCOc2c(-c3ccc4c(c3)CC(=O)N4)c(C)nn2C)c(C=O)[nH]1] | | 450.2 (M + 1)⁺ |
| N-22 | 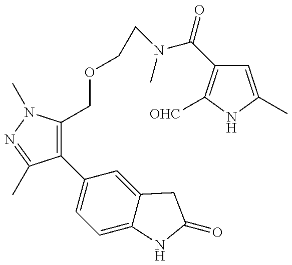 [SMILES: Cc1cc(C(=O)N(C)CCOCc2c(-c3ccc4c(c3)CC(=O)N4)c(C)nn2C)c(C=O)[nH]1] | | 450.2 (M + 1)⁺ |
| N-23 | 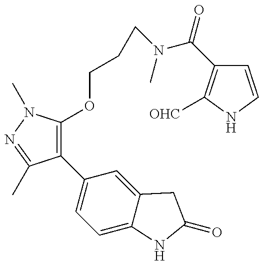 [SMILES: Cc1nn(C)c(OCCCN(C)C(=O)c2cc[nH]c2C=O)c1-c1ccc2c(c1)CC(=O)N2] | | 436.2 (M + 1)⁺ |
| N-24 | 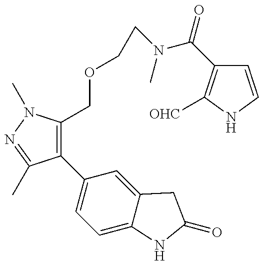 [SMILES: Cc1nn(C)c(COCCN(C)C(=O)c2cc[nH]c2C=O)c1-c1ccc2c(c1)CC(=O)N2] | | 436.2 (M + 1)⁺ |
| N-25 | 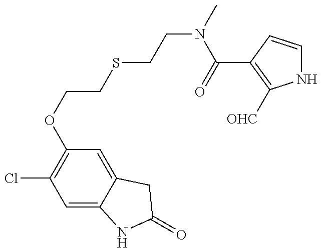 [SMILES: CN(CCSCCOc1cc2c(cc1Cl)NC(=O)C2)C(=O)c1cc[nH]c1C=O] | 12.38 (s, 1H), 10.30 (s, 1H), 9.63- 9.52 (m, 1H), 6.82 (s, 1H), 6.38 (s, 1H), 4.20-4.12 (m, 2H), 3.47 (s, 2H), 3.03-2.83 (m, 8H), 2.70-2.70 (m, 3H). | 422.0 (M + 1)⁺ |
| N-26 | 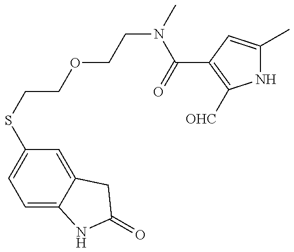 [SMILES: Cc1cc(C(=O)N(C)CCOCCSc2ccc3c(c2)CC(=O)N3)c(C=O)[nH]1] | | 402.3 (M + 1)⁺ |
| N-27 | 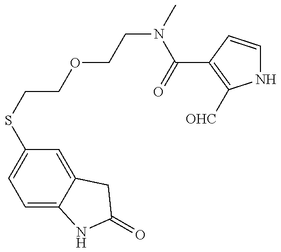 [SMILES: CN(CCOCCSc1ccc2c(c1)CC(=O)N2)C(=O)c1cc[nH]c1C=O] | | 388.1 (M + 1)⁺ |
| N-30 | 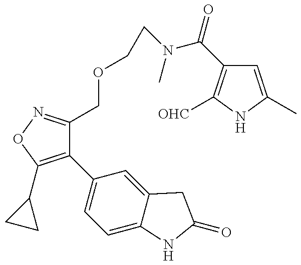 [SMILES: Cc1cc(C(=O)N(C)CCOCc2noc(C3CC3)c2-c2ccc3c(c2)CC(=O)N3)c(C=O)[nH]1] | | 463.2 (M + 1)⁺ |
| N-31 | 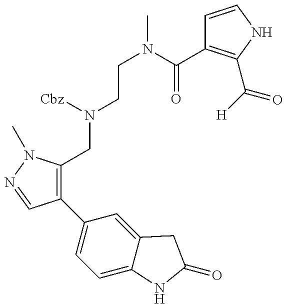 [SMILES: CN(CCN(Cc1c(-c2ccc3c(c2)CC(=O)N3)cnn1C)C(=O)OCc1ccccc1)C(=O)c1cc[nH]c1C=O] | | 555.3 (M + 1)⁺ |
| N-32 | 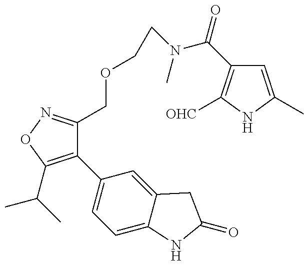 [SMILES: Cc1cc(C(=O)N(C)CCOCc2noc(C(C)C)c2-c2ccc3c(c2)CC(=O)N3)c(C=O)[nH]1] | | 465.2 (M + 1)⁺ |
| N-33 | 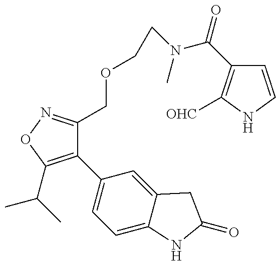 [SMILES: CC(C)c1onc(COCCN(C)C(=O)c2cc[nH]c2C=O)c1-c1ccc2c(c1)CC(=O)N2] | | 451.2 (M + 1)⁺ |
| N-34 | 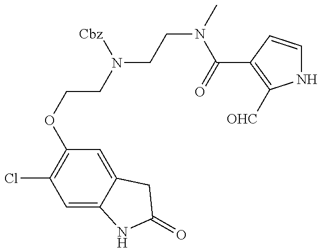 [SMILES: CN(CCN(CCOc1cc2c(cc1Cl)NC(=O)C2)C(=O)OCc1ccccc1)C(=O)c1cc[nH]c1C=O] | | 539.2 (M + 1)⁺ |
| N-35 | 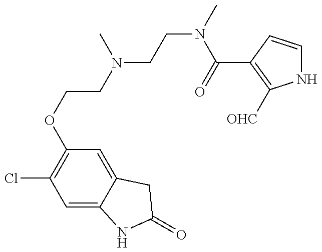 [SMILES: CN(CCOc1cc2c(cc1Cl)NC(=O)C2)CCN(C)C(=O)c1cc[nH]c1C=O] | | 419.0 (M + 1)⁺ |
| N-36 | 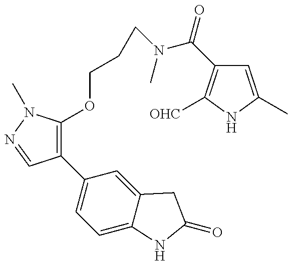 [SMILES: Cc1cc(C(=O)N(C)CCCOc2c(-c3ccc4c(c3)CC(=O)N4)cnn2C)c(C=O)[nH]1] | | 458.1 (M + Na)⁺ |
| N-37 | 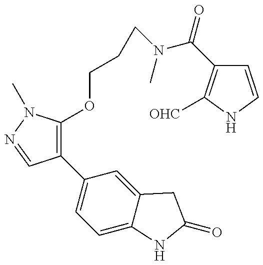 [SMILES: CN(CCCOc1c(-c2ccc3c(c2)CC(=O)N3)cnn1C)C(=O)c1cc[nH]c1C=O] | | 422.1 (M + 1)⁺ |
| N-38 | 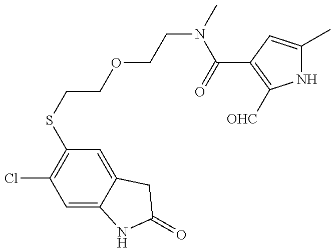 [SMILES: Cc1cc(C(=O)N(C)CCOCCSc2cc3c(cc2Cl)NC(=O)C3)c(C=O)[nH]1] | | 436.1 (M + 1)⁺ |
| N-39 | 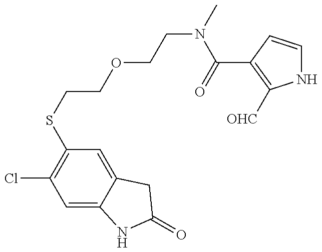 [SMILES: CN(CCOCCSc1cc2c(cc1Cl)NC(=O)C2)C(=O)c1cc[nH]c1C=O] | | 422.1 (M + 1)⁺ |

### Preparation of [19a(20)Z]-2,5-dimethyl-6,7,9,10-tetrahydro-1H-15,17-(ethanediylidene)pyrazolo[1,5-d]dipyrrolo[3,4-h:2′,3′-k][1,4,14]oxadiazacyclohexadecine-4,19(5H,18H)-dione (41) according to General Method O

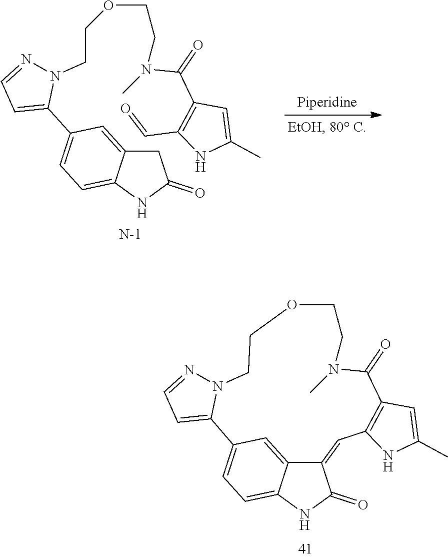

To a solution of N-1 (110 mg, 0.25 mmol, 1 eq) in EtOH (30 mL) was added piperidine (43.0 mg, 0.50 mmol, 2 eq). The mixture was stirred at 80° C. for 1 h. The reaction mixture was cooled and concentrated in vacuum. The crude product was triturated with MeOH (5 mL) at 25° C. for 10 min to 41 (42.2 mg, 0.100 mmol, 40% yield) as an orange solid. ¹H NMR (400 MHz, DMSO-d₆) δ (ppm) 12.62 (s, 1H), 11.08 (s, 1H), 7.95 (s, 1H), 7.52 (s, 1H), 7.40 (s, 1H), 7.36 (dd, J=8.0, 1.6 Hz, 1H), 7.00 (d, J=8.0 Hz, 1H), 6.46 (d, J=2.0 Hz, 1H), 6.24 (d, J=2.0 Hz, 1H), 4.40-4.27 (m, 3H), 4.18-4.16 (m, 1H), 4.04-3.91 (m, 2H), 3.70-3.68 (m, 1H), 3.18-3.07 (m, 1H), 2.98 (s, 3H), 2.41 (s, 3H). LCMS: m/z 418.2 (M+1)⁺.

Examples 42, 91, 92, 124-158, and 160-171 were prepared following similar procedures as 41 from starting material N₂-N₃₉, respectively. For 42, 125, 127, 139, 145, 160, and 163, the Cbz-protecting group is removed after cyclization step as shown below:

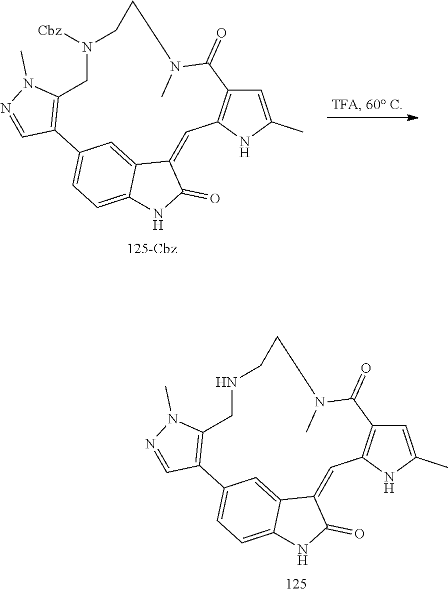 [SMILES: Cc1cc2c([nH]1)/C=C1\C(=O)Nc3ccc(cc31)-c1cnn(C)c1CN(C(=O)OCc1ccccc1)CCN(C)C2=O ; Cc1cc2c([nH]1)/C=C1\C(=O)Nc3ccc(cc31)-c1cnn(C)c1CNCCN(C)C2=O]

The mixture of 125-Cbz (65.0 mg, 0.12 mmol, 1 eq) in TFA (4 mL) was stirred at 60° C. for 16 h. On completion, the mixture was concentrated in vacuum. The residue was dissolved in sat. NaHCO₃ (aq. 30 mL) and lyophilized to afford solid. The solid was suspended in DCM/MeOH (10:1), filtered, and concentrated in vacuum. The residue was purified by silica gel column (DCM: MeOH=1:0-100:7) to afford 125 (3.3 mg, 6.4% yield) as orange powder.

For 133-138, the amides are synthesized after hydrolysis of the ester 132 followed by amide coupling with the corresponding amine and deprotection of Boc-protecting group if it is necessary as shown below:

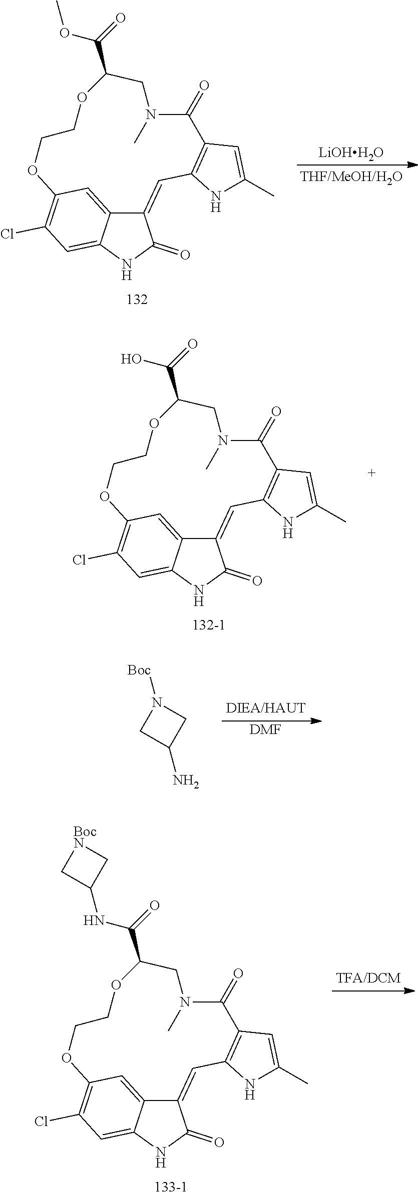

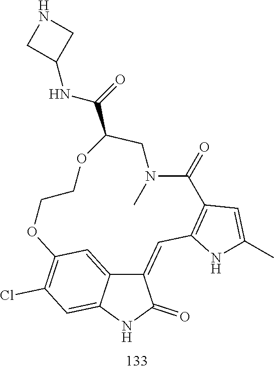 [SMILES: Cc1cc2c([nH]1)/C=C1\C(=O)Nc3cc(Cl)c(cc31)OCCO[C@@H](C(=O)NC1CNC1)CN(C)C2=O]

Step 1. To a solution of 132 (100 mg, 0.217 mmol, 1 eq.) in THF (1 mL), MeOH (1 mL) and H₂O (0.5 mL) was added LiOH·H₂O (27.4 mg, 0.652 mmol, 3 eq.). The mixture was stirred at 15° C. for 3 hours. The mixture was concentrated in vacuum to give 132-1 (115 mg, crude) as yellow solid. LC-MS: m/z 446.0 (M+1)⁺.

Step 2. To a solution of 132-1 (50.0 mg, 0.112 mmol, 1 eq.) and tert-butyl 3-aminoazetidine-1-carboxylate (23.2 mg, 0.134 mmol, 1.2 eq.), DIEA (43.5 mg, 0.336 umol, 3 eq.) in DMF (10 mL) was added HATU (51.2 mg, 0.135 mmol, 1.2 eq.) at 0° C. The mixture was stirred at 15° C. for 0.5 hours. The mixture was diluted with water (50 mL) and extratced with EtOAc (20 mL*3). The combined organic layer was dried over anhydrous Na₂SO₄, filtered and the filtrate was concentrated in vacuum. The residue was purified by prep-HPLC to provide 133-1 (19.0 mg, 30% yield) as a yellow solid. LC-MS: m/z 600.5 (M+1)⁺.

Step 3. A mixture of 133-1(19.0 mg, 0.032 mmol, 1 eq.) in DCM (1 mL) was added TFA (1 mL). The mixture was stirred at 15° C. for 3 hours. The mixture was concentrated in vacuum and the residue was purified by combi flash (4 g silica gel column, MeOH in DCM from 0% to 20%) to provide 133 (7.99 mg) as a yellow solid.

144 was oxidized to 148 and 149, respectively as shown below:

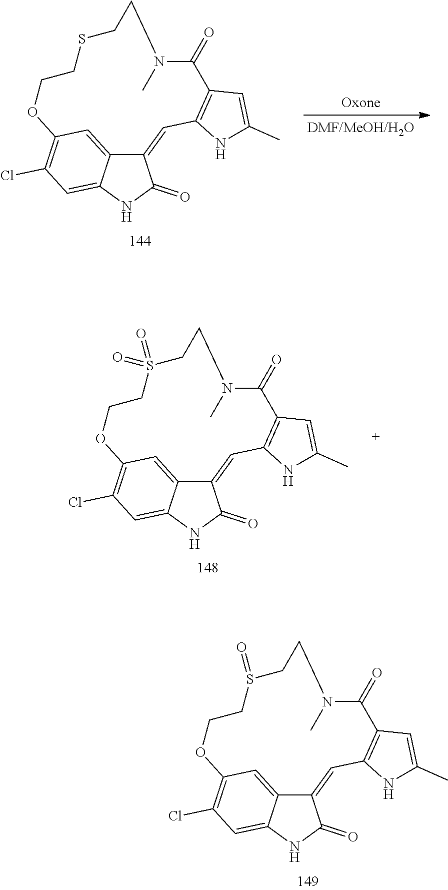 [SMILES: Cc1cc2c([nH]1)/C=C1\C(=O)Nc3cc(Cl)c(cc31)OCCSCCN(C)C2=O ; Cc1cc2c([nH]1)/C=C1\C(=O)Nc3cc(Cl)c(cc31)OCCS(=O)(=O)CCN(C)C2=O ; Cc1cc2c([nH]1)/C=C1\C(=O)Nc3cc(Cl)c(cc31)OCCS(=O)CCN(C)C2=O]

To a solution of 144 (20 mg, 47.9 umol, 1 eq) in DMF (2 mL), MeOH (2 mL) and H₂O (2 mL) was added oxone (588 mg, 0.957 mmol, 20 eq). The mixture was stirred at 25° C. for 16 hours. On completion, the mixture was filtered and the solid was triturated with H₂O and MeOH and filtered to provide 148 (1.2 mg) as a light-yellow solid product. The filtrate was evaporated and purified by prep-HPLC to provide 149 (2.3 mg) as a light-yellow solid product.

125 was converted to 152 or 156 via reductive amination reaction using acetaldehyde or acetone as shown below for 152:

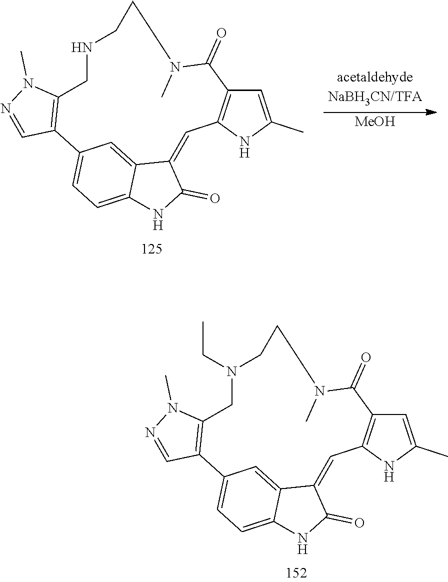 [SMILES: "MeOH" ; Cc1cc2c([nH]1)/C=C1\C(=O)Nc3ccc(cc31)-c1cnn(C)c1CNCCN(C)C2=O ; CCN1CCN(C)C(=O)c2cc(C)[nH]c2/C=C2\C(=O)Nc3ccc(cc32)-c2cnn(C)c2C1]

To a solution of 125 (393 mg, 0.942 mmol, 1 eq) in MeOH (15 mL) was added acetaldehyde (2.60 g, 23.5 mmol, 3.31 mL, 40% purity, 25 eq) and NaBH₃CN (296 mg, 4.71 mmol, 5 eq), and then TFA (644 mg, 5.66 mmol, 6 eq). The mixture was stirred at 25° C. for 16 hr. On completion, the mixture was concentrated and purified by flash silica gel chromatography (12 g silica gel column, DCM in MeOH from 0% to 100%) to give 152 (5.89 mg, 12.7 umol, 1.35% yield) as orange solid.

154 and 170 were oxidized to 155 and 171, respectively, using the method as shown below:

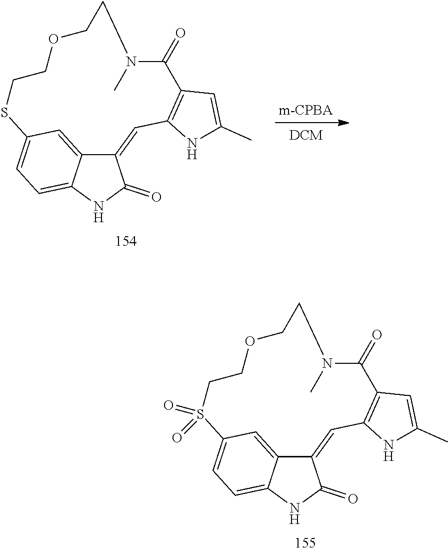 [SMILES: Cc1cc2c([nH]1)/C=C1\C(=O)Nc3ccc(cc31)SCCOCCN(C)C2=O ; Cc1cc2c([nH]1)/C=C1\C(=O)Nc3ccc(cc31)S(=O)(=O)CCOCCN(C)C2=O]

To a mixture of 154 (20 mg, 0.052 mmol, 1 eq) in DCM (3 mL) was added m-CPBA (22.0 mg, 0.104 mmol, 858% purity, 2 eq) at 0° C. The mixture was stirred at 25° C. for 1 hour, quenched by addition of sat. NaHCO₃ (1 mL), and then extracted with DCM (5 mL*3). The combined organic layers were dried over Na₂SO₄, filtered and concentrated under reduced pressure. The residue was purified by column chromatography (SiO₂, Dichloromethane: Methanol=100/0 to 30/1) followed by re-crystallization from MeOH (1 mL) to afford 155 (8.21 mg, 34.5% yield) as an orange solid.

| Ex | | | |
|---|---|---|---|
| # | Structure | ¹H NMR (400 MHz, DMSO-d₆) δ ppm | MS m/z |
| 41 | 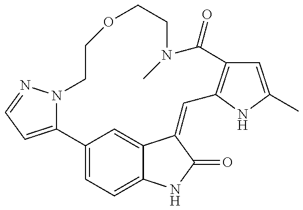 [SMILES: Cc1cc2c([nH]1)/C=C1\C(=O)Nc3ccc(cc31)-c1ccnn1CCOCCN(C)C2=O] | 12.62 (s, 1H), 11.08 (s, 1H), 7.95 (s, 1H), 7.52 (s, 1H), 7.40 (s, 1H), 7.36 (dd, J = 8.0, 1.6 Hz, 1H), 7.00 (d, J = 8.0 Hz, 1H), 6.46 (d, J = 2.0 Hz, 1H), 6.24 (d, J = 2.0 Hz, 1H), 4.40-4.27 (m, 3H), 4.18-4.16 (m, 1H), 4.04-3.91 (m, 2H), 3.70-3.68 (m, 1H), 3.18-3.07 (m, 1H), 2.98 (s, 3H), 2.41 (s, 3H) | 418.2 (M + 1)⁺ |
| 42 | 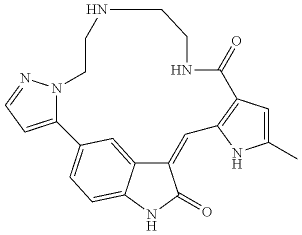 [SMILES: Cc1cc2c([nH]1)/C=C1\C(=O)Nc3ccc(cc31)-c1ccnn1CCNCCNC2=O] | 13.12 (s, 1H), 8.26 (s, 1H), 8.09-7.92 (m, 2H), 7.49 (s, 1H), 7.38 (d, J = 7.5 Hz, 1H), 7.24-7.17 (m, 1H), 7.00 (d, J = 8.0 Hz, 1H), 6.69-6.61 (m, 1H), 6.46 (s, 1H), 6.32 (s, 1H), 5.32 (s, 1H), 4.25 (s, 2H), 3.13 (d, J = 5.8 Hz, 2H), 2.79 (s, 2H), 2.38 (s, 3H), 2.05-1.93 (m, 2H) | 403.2 (M + 1)⁺ |
| 91 | 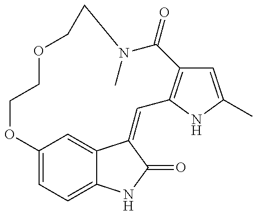 [SMILES: Cc1cc2c([nH]1)/C=C1\C(=O)Nc3ccc(cc31)OCCOCCN(C)C2=O] | 11.40 (s, 1H), 10.51 (s, 1H), 7.84 (d, J = 2.4 Hz, 1H), 7.40 (s, 1H), 6.73 (d, J = 8.0 Hz, 1H), 6.63 (dd, J = 8.4, 6.4 Hz, 1H), 6.23 (d, J = 2.4 Hz, 1H), 4.50 (dt, J = 8.4, 6.4 Hz, 1H), 4.32 (s, 2H), 3.80 (s, 2H), 3.75-3.66 (m, 1H), 3.69-3.62 (m, 1H), 3.00 (dd, J = 14.4, 4.8 Hz, 1H), 2.85 (s, 3H), 2.39 (s, 3H) | 368.3 (M + 1)⁺ |
| 92 | 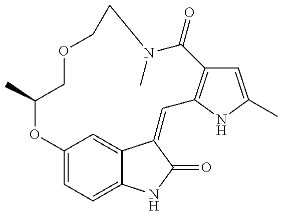 | 11.37 (s, 1H), 10.25 (s, 1H), 7.82-7.56 (m, 1H), 7.42 (s, 1H), 6.73 (d, J = 8.0 Hz, 1H), 6.60 (d, J = 7.2 Hz, 1H), 6.20 (s, 1H), 4.59-4.42 (m, 1H), 4.40-4.12 (m, 1H), 3.81-3.54 (m, 4H), 3.14-2.66 (m, 3H), 3.10-2.92 (m, 1H), 2.38 (s, 3H), 1.43 (s, 3H). | 382.4 (M + 1)⁺ |
| 124 | 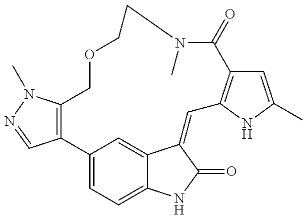 [SMILES: Cc1cc2c([nH]1)/C=C1\C(=O)Nc3ccc(cc31)-c1cnn(C)c1COCCN(C)C2=O] | 11.34 (s, 1H), 10.74 (s, 1H), 7.98 (s, 1H), 7.73 (s, 1H), 7.47 (s, 1H), 7.34 (dd, J = 8.0, 1.2 Hz, 1H), 6.89 (d, J = 7.8 Hz, 1H), 6.25 (d, J = 2.4 Hz, 1H), 4.66 (d, J = 12.0 Hz, 1H), 4.53-4.51 (m, 1H), 4.42 (d, J = 12.0 Hz, 1H), 4.04-3.97 (m, 1H), 3.91 (s, 3H), 3.89-3.83 (m, 1H), 3.07 (m, 1H), 2.99 (s, 3H), 2.40 (s, 3H) | 418.4 (M + 1)⁺ |
| 125 | 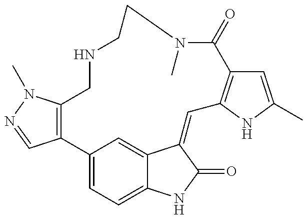 [SMILES: Cc1cc2c([nH]1)/C=C1\C(=O)Nc3ccc(cc31)-c1cnn(C)c1CNCCN(C)C2=O] | 11.57 (s, 1H), 10.71 (s, 1H), 8.87 (s, 1H), 7.69 (s, 1H), 7.51 (s, 1H), 7.29 (d, J = 8.0 Hz, 1H), 6.84 (d, J = 8.0 Hz, 1H), 6.22 (s, 1H), 4.50 (d, J = 12.8 Hz, 1H), 3.91 (s, 3H), 3.84 (d, J = 12.8 Hz, 1H), 3.53 (t, J = 12.0 Hz, 1H), 3.12 (s, 1H), 3.06 (s, 3H), 2.84 (s, 3H), 2.38 (s, 3H) | 417.0 (M + 1)⁺ |
| 126 | 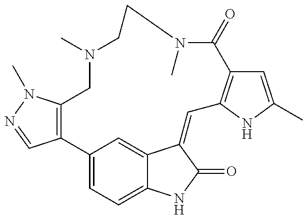 [SMILES: Cc1cc2c([nH]1)/C=C1\C(=O)Nc3ccc(cc31)-c1cnn(C)c1CN(C)CCN(C)C2=O] | 11.64 (s, 1H), 10.74 (s, 1H), 8.63 (s, 1H), 7.83 (s, 1H), 7.48 (s, 1H), 7.37 (d, J = 8.0 Hz, 1H), 6.85 (d, J = 8.0 Hz, 1H), 6.23 (d, J = 2.0 Hz, 1H), 4.65-4.48 (m, 1H), 3.90 (s, 3H), 3.86-3.69 (m, 1H), 3.54-3.37 (m, 1H), 3.20-3.06 (m, 2H), 3.01 (s, 3H), 2.56-2.52 (m, 1H), 2.39 (s, 3H), 2.21 (s, 3H) | 431.0 (M + 1)⁺ |
| 127 | 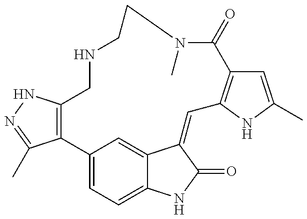 [SMILES: Cc1cc2c([nH]1)/C=C1\C(=O)Nc3ccc(cc31)-c1c(C)n[nH]c1CNCCN(C)C2=O] | 12.48 (s, 1H), 11.66 (s, 1H), 10.71 (s, 1H), 9.06 (s, 1H), 7.53 (s, 1H), 7.19 (d, J = 4.4 Hz, 1H), 6.87 (d, J = 8.0 Hz, 1H), 6.21 (s, 1H), 4.42 (d, J = 12.0 Hz, 1H), 3.72 (d, J = 10.8 Hz, 1H), 3.40 (d, J = 10.8 Hz, 1H), 3.05 (s, 3H), 3.03-2.97 (m, 1H), 2.96-2.87 (m, 1H), 2.84-2.71 (m, 1H), 2.53-2.51 (m, 1H), 2.38 (s, 3H), 2.37 (s, 3H) | 417.1 (M + 1)⁺ |
| 128 | 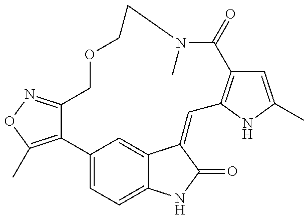 [SMILES: Cc1cc2c([nH]1)/C=C1\C(=O)Nc3ccc(cc31)-c1c(noc1C)COCCN(C)C2=O] | 11.40 (s, 1H), 10.85 (s, 1H), 8.17 (s, 1H), 7.50 (s, 1H), 7.32 (d, J = 8.4 Hz, 1H), 6.98 (d, J = 8.4 Hz, 1H), 6.26 (s, 1H), 4.62-4.38 (m, 4H), 3.91 (s, 2H), 2.99 (s, 3H), 2.60 (s, 3H), 2.40 (s, 3H) | 419.2 (M + 1)⁺ |
| 129 | 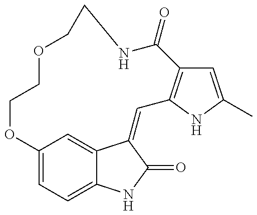 [SMILES: Cc1cc2c([nH]1)/C=C1\C(=O)Nc3ccc(cc31)OCCOCCNC2=O] | 11.68 (s, 1H), 10.52 (s, 1H), 8.27 (t, J = 4.4 Hz, 1H), 8.04 (s, 1H), 7.90 (d, J = 2.4 Hz, 1H), 6.75 (d, J = 8.0 Hz, 1H), 6.63 (dd, J = 8.0, 2.4 Hz, 1H), 6.27 (d, J = 2.0 Hz, 1H), 4.34 (s, 2H), 3.72 (s, 2H), 3.67- 3.62 (m, 2H), 3.51-3.44 (m, 2H), 2.37 (s, 3H). | 354.1 (M + 1)⁺ |
| 130 | 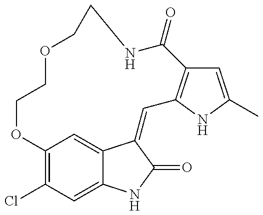 [SMILES: Cc1cc2c([nH]1)/C=C1\C(=O)Nc3cc(Cl)c(cc31)OCCOCCNC2=O] | 11.64 (s, 1H), 10.63 (s, 1H), 8.41-8.28 (m, 1H), 8.03 (d, J = 7.2 Hz, 2H), 6.87 (s, 1H), 6.30 (d, J = 2.8 Hz, 1H), 4.46 (d, J = 1.6 Hz, 2H), 3.76 (s, 2H), 3.65 (d, J = 4.0 Hz, 2H), 3.49-3.47 (m, 2H), 2.37 (s, 3H) | 388.0 (M + 1)⁺ |
| 131 | 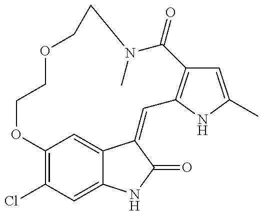 [SMILES: Cc1cc2c([nH]1)/C=C1\C(=O)Nc3cc(Cl)c(cc31)OCCOCCN(C)C2=O] | 11.37 (s, 1H), 10.62 (s, 1H), 8.04-7.86 (m, 1H), 7.39 (s, 1H), 6.85 (s, 1H), 6.24 (s, 1H), 4.62-4.34 (m, 3H), 3.88-3.74 (m, 3H), 3.72-3.63 (m, 1H), 3.04-2.96 (m, 1H), 2.83 (s, 3H), 2.39 (s, 3H) | 402.1 (M + 1)⁺ |
| 132 | 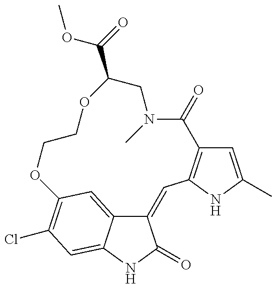 [SMILES: COC(=O)[C@H]1CN(C)C(=O)c2cc(C)[nH]c2/C=C2\C(=O)Nc3cc(Cl)c(cc32)OCCO1] | 11.21 (s, 1 H), 8.55 (s, 1 H), 7.95 (s, 1 H), 7.70 (s, 1 H), 6.84 (s, 1 H), 6.25 (s, 1 H), 5.10 (dd, J = 14.4, 1.6 Hz, 1 H), 4.89- 4.81 (m, 1 H), 4.24-4.20 (m, 1 H), 4.19- 4.15 (m, 1 H), 4.07-4.01 (m, 1 H), 3.98- 3.90 (m, 1 H), 3.69 (s, 3 H) 3.22 (dd, J = 14.4, 8.0 Hz, 1 H), 3.17 (s, 3 H), 2.40 (s, 3 H) | 460.4 (M + 1)⁺ |
| 133 | 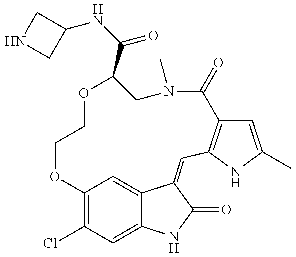 [SMILES: Cc1cc2c([nH]1)/C=C1\C(=O)Nc3cc(Cl)c(cc31)OCCO[C@@H](C(=O)NC1CNC1)CN(C)C2=O] | ¹H NMR (400 MHz, MeOD-d₄) δ = 8.72 (s, 1H), 8.10 (s, 1 H), 6.84 (s, 1H), 6.21 (s, 1H), 4.93 (dd, J = 14.8, 2.0 Hz, 1H), 4.77 (dd, J = 14.0, 6.0 Hz, 1H), 4.55- 4.43 (m, 1H), 4.30-4.22 (m, 2H), 4.21- 4.16 (m, 3H), 4.11 (d, J = 14.0 Hz, 1H), 3.98-3.89 (m, 2H), 3.35 (dd, J = 14.8, 3.6 Hz, 1H), 3.13 (s, 3H), 2.40 (s, 3H) | 500.1 (M + 1)⁺ |
| 134 | 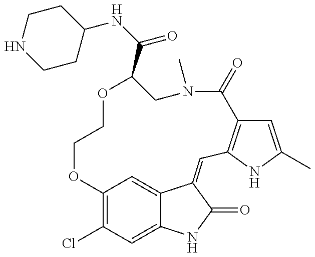 [SMILES: Cc1cc2c([nH]1)/C=C1\C(=O)Nc3cc(Cl)c(cc31)OCCO[C@@H](C(=O)NC1CCNCC1)CN(C)C2=O] | ¹H NMR (400 MHz, MeOD-d₄) δ = 8.71 (s, 1H), 8.51 (s, 1H), 8.18 (s, 1H), 6.84 (s, 1H), 6.22 (s, 1H), 4.97-4.88 (m, 2H), 4.75 (dd, J = 14.2, 6.0 Hz, 1H), 4.18-4.07 (m, 2H), 4.01-3.86 (m, 2H), 3.84-3.74 (m, 1H), 3.41-3.34 (m, 2H), 3.13 (s, 3H), 3.07-2.95 (m, 2H), 2.41 (s, 3H), 2.23-2.01 (m, 2H), 1.80-1.61 (m, 2H) | 528.3 (M + 1)⁺ |
| 135 | 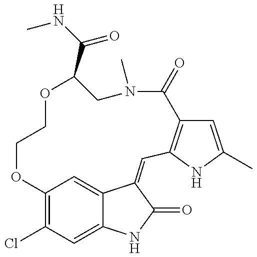 [SMILES: CNC(=O)[C@H]1CN(C)C(=O)c2cc(C)[nH]c2/C=C2\C(=O)Nc3cc(Cl)c(cc32)OCCO1] | ¹H NMR (400 MHz, MeOD-d₄) δ = 8.42 (s, 1H), 7.98 (s, 1H), 6.85 (s, 1H), 6.23 (s, 1H), 4.61-4.55 (m, 1H), 4.39-4.27 (m, 1H), 4.18 (dd, J = 4.8, 3.3 Hz, 1H), 3.97-3.90 (m, 1H), 3.89-3.81 (m, 1H), 3.61 (q, J = 14.0, 7.2 Hz, 1H), 3.23 (dd, J = 14.0, 2.8 Hz, 1H), 3.03 (s, 3H), 2.73 (s, 3H), 2.41 (s, 3H). | 459.2 (M + 1)⁺ |
| 136 | 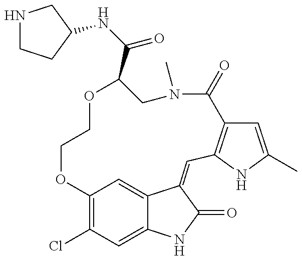 | ¹H NMR (400 MHz, MeOD-d₄) δ = 8.79 (s, 1H), 8.50 (s, 1H), 8.09 (s, 1H), 6.84 (s, 1H), 6.23 (s, 1H), 4.96 (dd, J = 14.8, 1.6 Hz, 1H), 4.78 (dd, J = 14.0, 2.0 Hz, 1H), 4.29-4.23 (m, 1H), 4.21 (d, J = 2.4 Hz, 1H), 4.09 (d, J = 14.4 Hz, 1H), 4.01- 3.95 (m, 2H), 3.52-3.44 (m, 1H), 3.40- 3.34 (m, 3H), 3.20 (dd, J = 12.4, 6.0 Hz, 1H), 3.15 (s, 3H), 2.40 (s, 3H), 2.28- 2.21 (m, 1H), 2.12-2.02 (m, 1H) | 514.2 (M + 1)⁺ |
| 137 | 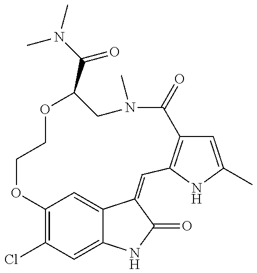 [SMILES: Cc1cc2c([nH]1)/C=C1\C(=O)Nc3cc(Cl)c(cc31)OCCO[C@@H](C(=O)N(C)C)CN(C)C2=O] | ¹H NMR (400 MHz, MeOD-d₄) δ = 8.88 (s, 1 H), 8.12 (s, 1 H), 6.82 (s, 1 H), 6.20 (s, 1 H), 4.96-4.89 (m, 1 H), 4.79-4.75 (m, 2 H), 4.11 (s, 1 H), 3.96-3.89 (m, 2 H), 3.28-3.25 (m, 1 H), 3.18 (s, 3 H), 3.11 (s, 4 H), 2.83 (s, 3 H), 2.40 (s, 3 H) | 473.4 (M + 1)⁺ |
| 138 | 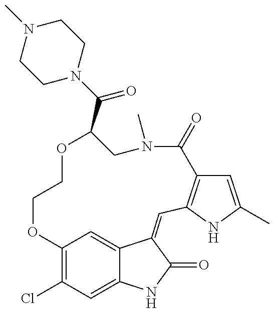 | ¹H NMR (400 MHz, MeOD-d₄) δ = 8.89 (s, 1H), 8.40 (s, 1H), 8.11 (s, 1H), 6.82 (s, 1H), 6.20 (s, 1H), 4.80-4.73 (m, 2H), 4.10-3.93 (m, 1H), 3.92-3.61 (m, 7H), 3.52-3.48 (m, 1H), 3.12 (s, 3H), 3.02- 2.98 (m, 1H), 2.81-2.73 (m, 2H), 2.58- 2.54 (m, 1H), 2.53 (s, 3H), 2.39 (s, 3H) | 528.4 (M + 1)⁺ |
| 139 | 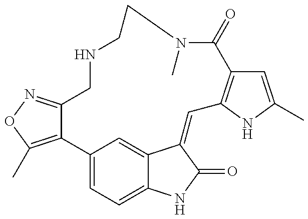 [SMILES: Cc1cc2c([nH]1)/C=C1\C(=O)Nc3ccc(cc31)-c1c(noc1C)CNCCN(C)C2=O] | 11.62 (s, 1H), 10.42 (s, 1H), 7.52 (s, 1H), 7.34-7.02 (m, 2H), 6.96 (d, J = 8.0 Hz, 1H), 6.24 (d, J = 9.2 Hz, 1H), 4.04 (s, 2H), 3.66-3.51 (m, 3H), 3.04-2.95 (m, 1H), 2.91 (s, 1H), 2.89 (d, J = 6.8 Hz, 3H), 2.59 (s, 1H), 2.29 (s, 2H), 2.26 (s, 3H). | 418.4 (M + 1)⁺ |
| 140 | 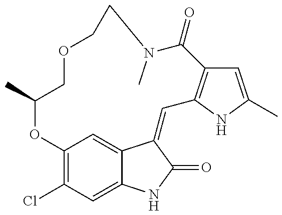 | 11.34 (s, 1H), 10.61 (d, J = 4.4 Hz, 1H), 7.94-7.70 (m, 1H), 7.37 (d, J = 3.6 Hz, 1H), 6.85 (d, J = 2.4 Hz, 1H), 6.24 (dd, J = 2.0, 7.2 Hz, 1H), 4.57-4.19 (m, 2H), 3.85-3.57 (m, 4H), 3.11 (s, 1H), 3.07- 2.93 (m, 1H), 2.67 (s, 2H), 2.39 (s, 3H), 1.47 (dd, J = 6.6, 17.5 Hz, 3H). | 416.3 (M + 1)⁺ |
| 141 | 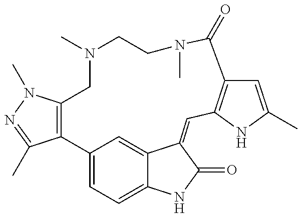 [SMILES: Cc1cc2c([nH]1)/C=C1\C(=O)Nc3ccc(cc31)-c1c(C)nn(C)c1CN(C)CCN(C)C2=O] | 11.72 (s, 1H), 10.74 (s, 1H), 8.61 (s, 1H), 7.47 (s, 1H), 7.26 (d, J = 8.4 Hz, 1H), 6.90 (d, J = 8.0 Hz, 1H), 6.22 (s, 1H), 4.69-4.49 (m, 1H), 3.83 (s, 3H), 3.66- 3.56 (m, 1H), 3.29-3.21 (m, 3H), 3.10- 2.93 (m, 4H), 2.39 (s, 3H), 2.34 (s, 3H), 2.21 (s, 3H). | 445.3 (M + 1)⁺ |
| 142 | 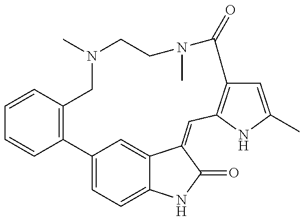 [SMILES: Cc1cc2c([nH]1)/C=C1\C(=O)Nc3ccc(cc31)-c1ccccc1CN(C)CCN(C)C2=O] | 11.67 (s, 1H), 10.84 (s, 1H), 9.05-8.72 (m, 1H), 7.68-7.49 (m, 2H), 7.42 (d, J = 4.8 Hz, 3H), 7.35-7.26 (m, 1H), 6.98 (d, J = 8.0 Hz, 1H), 6.24 (s, 1H), 4.79-4.39 (m, 1H), 3.98-3.86 (m, 1H), 3.33-3.28 (m, 3H), 3.21-2.98 (m, 4H), 2.40 (s, 3H), 2.22 (s, 3H). | 427.1 (M + 1)⁺ |
| 143 | 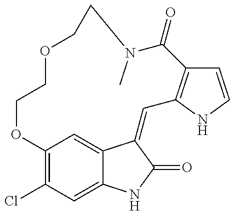 [SMILES: CN1CCOCCOc2cc3c(cc2Cl)NC(=O)/C3=C\c2[nH]ccc2C1=O] | 11.60 (s, 1H), 10.64 (s, 1H), 7.99 (s, 1H), 7.45 (s, 1H), 7.36-7.33 (m, 1H), 6.86 (s, 1H), 6.46-6.43 (s, 1H), 4.56-4.49 (m, 1H), 4.49-4.40 (m, 2H), 3.85-3.78 (m, 2H), 3.77-3.71 (m, 1H), 3.73-3.67 (m, 1H), 3.02-2.85 (m, 1H), 2.83 (s, 3H). | 388.0 (M + 1)⁺ |
| 144 | 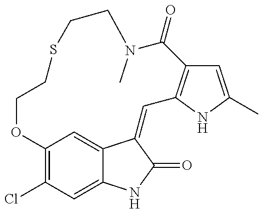 [SMILES: Cc1cc2c([nH]1)/C=C1\C(=O)Nc3cc(Cl)c(cc31)OCCSCCN(C)C2=O] | 11.84 (s, 1H), 10.69 (s, 1H), 8.08 (s, 1H), 7.44 (s, 1H), 6.85 (s, 1H), 6.21 (d, J = 2.0 Hz, 1H), 4.63-4.51 (m, 2H), 4.23 (s, 1H), 3.38-3.20 (m, 2H), 3.18-3.15 (m, 1H), 3.05-2.09 (m, 2), 2.82 (s, 3H), 2.40 (s, 3H). | 418.1 (M + 1)⁺ |
| 145 | 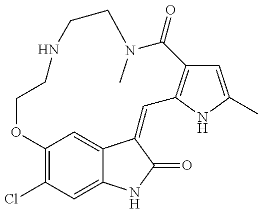 [SMILES: Cc1cc2c([nH]1)/C=C1\C(=O)Nc3cc(Cl)c(cc31)OCCNCCN(C)C2=O] | 11.57 (s, 1H), 10.59 (s, 1H), 8.95 (s, 1H), 7.53 (s, 1H), 6.81 (s, 1H), 6.22 (d, J = 2.0 Hz, 1H), 5.32 (t, J = 4.8 Hz, 1H), 4.48- 4.29 (m, 2H), 4.24-4.15 (m, 1H), 3.17 (d, J = 5.2 Hz, 1H), 3.04-2.92 (m, 1H), 2.91-2.70 (m, 8H), 2.38 (s, 3H) | 401.4 (M + 1)⁺ |
| 146 | 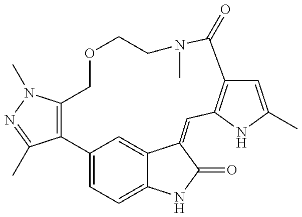 [SMILES: Cc1cc2c([nH]1)/C=C1\C(=O)Nc3ccc(cc31)-c1c(C)nn(C)c1COCCN(C)C2=O] | 10.60 (s, 1H), 10.76 (s, 2H), 7.79 (s, 1H), 7.30-7.25 (m, 2H), 6.89 (d, J = 8.0 Hz, 1H), 6.21 (d, J = 8.0 Hz, 1H), 4.36-4.33 (m, 4H), 3.66 (s, 3H), 2.86 (s, 3H), 2.74- 2.72 (m, 1H), 2.55-2.45 (m, 4H), 2.43 (s, 3H), 2.05-2.01 (m, 2H). | 432.2 (M + 1)⁺ |
| 147 | 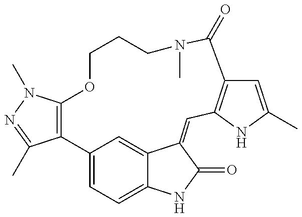 [SMILES: Cc1cc2c([nH]1)/C=C1\C(=O)Nc3ccc(cc31)-c1c(C)nn(C)c1OCCCN(C)C2=O] | 11.41 (s, 1H), 10.76 (s, 1H), 7.90 (d, J = 1.2 Hz, 1H), 7.46 (s, 1H), 7.26 (d, J = 8.0 Hz, 1H), 6.93 (d, J = 8.0 Hz, 1H), 6.24 (d, J = 2.0 Hz, 1H), 4.61-4.49 (m, 2H), 4.29-4.26 (m, 1H), 3.98-3.95 (m, 1H), 3.92 (s, 3H), 3.80-3.79 (m, 1H), 3.10- 3.05 (m, 1H), 3.04 (s, 3H), 2.39 (s, 3H), 2.30 (s, 3H). | 432.2 (M + 1)⁺ |
| 148 | 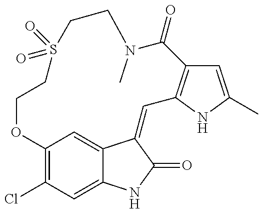 [SMILES: Cc1cc2c([nH]1)/C=C1\C(=O)Nc3cc(Cl)c(cc31)OCCS(=O)(=O)CCN(C)C2=O] | 11.84 (s, 1H), 10.74-10.65 (m, 1H), 7.80 (s, 1H), 7.25 (s, 1H), 6.87 (s, 1H), 6.21 (d, J = 1.6 Hz, 1H), 4.90-4.81 (m, 1H), 4.74 (t, J = 5.6 Hz, 2H), 4.14-3.92 (m, 3H), 3.78-3.69 (m, 1H), 3.31-3.30 (m, 1H), 2.78 (s, 3H), 2.40 (s, 3H). | 450.0 (M + 1)⁺ |
| 149 | 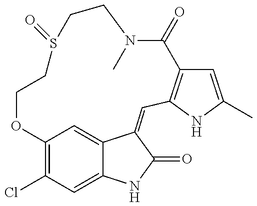 [SMILES: Cc1cc2c([nH]1)/C=C1\C(=O)Nc3cc(Cl)c(cc31)OCCS(=O)CCN(C)C2=O] | 11.87-11.65 (m, 1H), 10.72 (s, 1H), 7.52- 7.37 (m, 1H), 7.25-7.10 (m, 1H), 6.89 (d, J = 15.4 Hz, 1H), 6.22 (d, J = 7.2 Hz, 1H), 4.96-4.70 (m, 2H), 4.65-4.41 (m, 1H), 3.27-2.88 (m, 5H), 2.79 (s, 3H), 2.40 (s, 3H). | 434.0 (M + 1)⁺ |
| 150 | 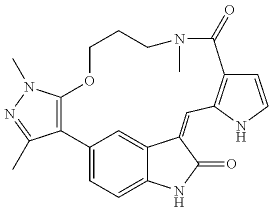 [SMILES: Cc1nn(C)c2c1-c1ccc3c(c1)/C(=C/c1[nH]ccc1C(=O)N(C)CCCO2)C(=O)N3] | 11.50 (m, 1H), 10.55 (s, 1H), 7.35 (s, 1H), 7.16 (d, J = 7.5 Hz, 1H), 6.95 (s, 1H), 6.88- 6.79 (m, 2H), 6.38 (s, 1H), 4.31-4.15 (m, 1H), 3.77 (s, 1H), 3.52 (s, 3H), 3.47 (d, J = 7.2 Hz, 1H), 3.22 (s, 1H), 2.80 (s, 3H), 2.14 (s, 3H), 1.85-1.58 (m, 2H). | 418.2 (M + 1)⁺ |
| 151 | 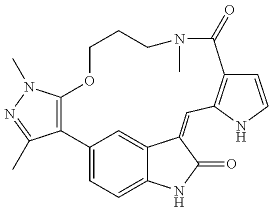 [SMILES: Cc1nn(C)c2c1-c1ccc3c(c1)/C(=C/c1[nH]ccc1C(=O)N(C)CCCO2)C(=O)N3] | 11.63 (s, 1H), 10.78 (s, 1H), 7.93 (s, 1H), 7.52 (s, 1H), 7.35 (t, J = 2.6 Hz, 1H), 7.29- 7.26 (m, 1H), 6.93 (d, J = 8.0 Hz, 1H), 6.45-6.40 (m, 1H), 4.61-4.49 (m, 2H), 4.29 (d, J = 12 Hz, 1H), 3.98-3.96 (m, 1H), 3.82-3.78 (m, 1H), 3.12-3.06 (m, 1H), 3.04 (s, 3H), 2.30 (s, 3H). | 418.2 (M + 1)⁺ |
| 152 | 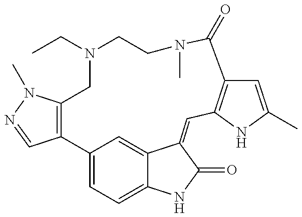 [SMILES: CCN1CCN(C)C(=O)c2cc(C)[nH]c2/C=C2\C(=O)Nc3ccc(cc32)-c2cnn(C)c2C1] | 11.66 (s, 1H), 10.74 (s, 1H), 8.79-8.60 (m, 1H), 7.83 (s, 1H), 7.52 (s, 1H), 7.38 (d, J = 8.0 Hz, 1H), 6.86 (d, J = 8.0 Hz, 1H), 6.23 (d, J = 2.4 Hz, 1H), 4.60 (dd, J = 13.6, 1.6 Hz, 1H), 3.93 (s, 3H), 3.72 (s, 2H), 3.35 (d, J = 1.0 Hz, 1H), 3.05-2.97 (m, 3H), 2.96-2.84 (m, 2H), 2.73-2.64 (m, 2H), 2.39 (s, 3H), 0.80 (t, J = 7.1 Hz, 3H). | 445.3 (M + 1)⁺ |
| 153 | 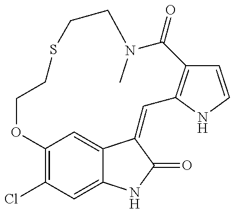 [SMILES: CN1CCSCCOc2cc3c(cc2Cl)NC(=O)/C3=C\c2[nH]ccc2C1=O] | 12.00 (s, 1H), 10.72 (s, 1H), 8.11 (s, 1H), 7.50 (s, 1H), 7.37 (t, J = 2.4 Hz, 1H), 6.87 (s, 1H), 6.41 (t, J = 2.4 Hz, 1H), 4.66-4.52 (m, 2H), 4.28-4.19 (m, 1H), 3.26-3.16 (m, 2H), 3.10-3.01 (m, 1H), 3.00-2.92 (m, 2H), 2.82 (s, 3H). | 404.1 (M + 1)⁺ |
| 154 | 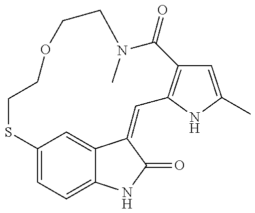 [SMILES: Cc1cc2c([nH]1)/C=C1\C(=O)Nc3ccc(cc31)SCCOCCN(C)C2=O] | 12.02 (br s, 1H), 10.80 (s, 1H), 7.61 (d, J = 1.6 Hz, 1H), 7.38 (s, 1H), 6.97 (dd, J = 8.0, 2.0 Hz, 1H), 6.81 (d, J = 8.0 Hz, 1H), 6.24 (d, J = 2.0 Hz, 1H), 4.57-4.46 (m, 1H), 3.96-3.89 (m, 1H), 3.82 (td, J = 5.2, 10.1 Hz, 1H), 3.74-3.65 (m, 2H), 3.31 (br s, 1H), 3.14-3.07 (m, 1H), 3.02- 2.94 (m, 4H), 2.39 (s, 3H) | 384.1 (M + 1)⁺ |
| 155 | 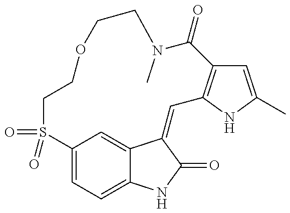 [SMILES: Cc1cc2c([nH]1)/C=C1\C(=O)Nc3ccc(cc31)S(=O)(=O)CCOCCN(C)C2=O] | 11.83 (br s, 1H), 11.24 (s, 1H), 8.26 (d, J = 1.2 Hz, 1H), 7.59 (dd, J = 8.0, 1.6 Hz, 1H), 7.45 (s, 1H), 7.07 (d, J = 8.0 Hz, 1H), 6.28 (d, J = 2.0 Hz, 1H), 4.55-4.43 (m, 1H), 4.19 (br s, 1H), 4.14-4.06 (m, 1H), 3.90 (br s, 2H), 3.86-3.75 (m, 2H), 3.04-2.95 (m, 1H), 2.87 (s, 3H), 2.41 (s, 3H) | 416.1 (M + 1)⁺ |
| 156 | 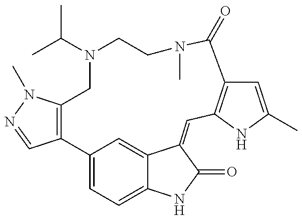 [SMILES: Cc1cc2c([nH]1)/C=C1\C(=O)Nc3ccc(cc31)-c1cnn(C)c1CN(C(C)C)CCN(C)C2=O] | 11.69 (s, 1H), 10.75 (s, 1H), 9.14-8.90 (m, 1H), 7.79 (s, 1H), 7.66 (s, 1H), 7.36 (d, J = 7.8 Hz, 1H), 6.86 (d, J = 8.0 Hz, 1H), 6.24 (d, J = 2.0 Hz, 1H), 3.88 (s, 3H), 3.76 (s, 2H), 2.99-2.92 (m, 2H), 2.89 (s, 3H), 2.52 (s, 2H), 2.39 (s, 3H), 1.32 (s, 1H), 1.11 (d, J = 7.0 Hz, 3H), 0.99 (d, J = 6.0 Hz, 3H). | 459.3 (M + 1)⁺ |
| 157 | 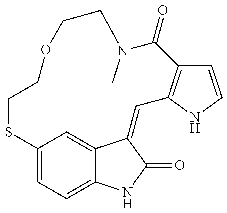 [SMILES: CN1CCOCCSc2ccc3c(c2)/C(=C/c2[nH]ccc2C1=O)C(=O)N3] | 12.17 (br s, 1H), 10.81 (br s, 1H), 7.65 (d, J = 1.2 Hz, 1H), 7.45 (s, 1H), 7.37 (t, J = 2.4 Hz, 1H), 7.00 (dd, J = 8.0, 1.6 Hz, 1H), 6.81 (d, J = 8.0 Hz, 1H), 6.44 (t, J = 2.4 Hz, 1H), 4.61-4.48 (m, 1H), 3.97- 3.89 (m, 1H), 3.84 (td, J = 10.0, 5.2 Hz, 1H), 3.77-3.66 (m, 2H), 3.37 (br s, 1H), 3.13 (td, J = 16.0, 4.8 Hz, 1H), 3.01- 2.94 (m, 4H) | 370.2 (M + 1)⁺ |
| 158 | 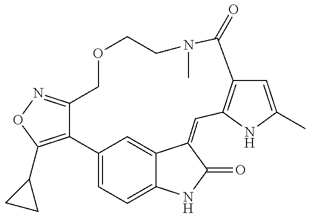 [SMILES: Cc1cc2c([nH]1)/C=C1\C(=O)Nc3ccc(cc31)-c1c(noc1C1CC1)COCCN(C)C2=O] | 11.41 (s, 1H), 10.85 (s, 1H), 8.17 (d, J = 1.2 Hz, 1H), 7.50 (d, J = 8.0 Hz, 2H), 6.98 (d, J = 8.0 Hz, 1H), 6.26 (s, 1H), 4.52-4.45 (m, 3H), 3.90-3.80 (m, 2H), 3.33-3.15 (m, 1H), 2.92 (s, 3H), 2.35 (s, 3H), 2.34-2.33 (m, 1H), 1.18-1.09 (m, 4H). | 445.2 (M + 1)⁺ |
| 160 | 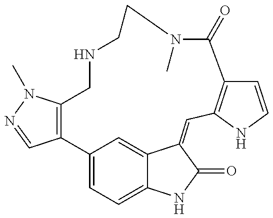 [SMILES: CN1CCNCc2c(cnn2C)-c2ccc3c(c2)/C(=C/c2[nH]ccc2C1=O)C(=O)N3] | 11.77 (s, 1H), 10.74 (s, 1H), 8.90 (s, 1H), 7.70 (s, 1H), 7.57 (s, 1H), 7.36- 7.29 (m, 2H), 6.86 (d, J = 8.0 Hz, 1H), 6.43 (t, J = 2.4 Hz, 1H), 4.57-4.45 (m, 1H), 3.91 (s, 3H), 3.84 (d, J = 12.0 Hz, 1H), 3.59-3.48 (m, 1H), 3.15-3.09 (m, 1H), 3.06 (s, 3H), 2.89-2.84 (m, 1H), 2.84-2.76 (m, 2H). | 403.2 (M + 1)⁺ |
| 161 | 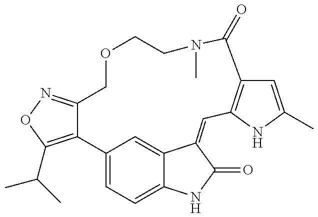 [SMILES: Cc1cc2c([nH]1)/C=C1\C(=O)Nc3ccc(cc31)-c1c(noc1C(C)C)COCCN(C)C2=O] | 11.42 (s, 1H), 10.85 (s, 1H), 8.14 (d, J = 1.2 Hz, 1H), 7.50 (s, 1H), 7.26 (dd, J = 1.6, 8.0 Hz, 1H), 6.99 (d, J = 8.0 Hz, 1H), 6.27 (d, J = 1.6 Hz, 1H), 4.59-4.39 (m, 3H), 3.94-3.85 (m, 2H), 3.52-3.44 (m, 1H), 3.09 (dd, J = 8.0, 14.2 Hz, 1H), 3.00 (s, 3H), 2.40 (s, 3H), 1.38 (d, J = 6.4 Hz, 3H), 1.28 (d, J = 6.4 Hz, 3H). | 447.2 (M + 1)⁺ |
| 162 | 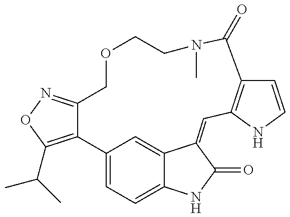 [SMILES: CC(C)c1onc2c1-c1ccc3c(c1)/C(=C/c1[nH]ccc1C(=O)N(C)CCOC2)C(=O)N3] | 11.64 (s, 1H), 10.87 (s, 1H), 8.16 (d, J = 1.2 Hz, 1H), 7.56 (s, 1H), 7.36 (t, J = 2.4 Hz, 1H), 7.28 (dd, J = 1.6, 8.0 Hz, 1H), 7.00 (d, J = 8.0 Hz, 1H), 6.47 (t, J = 2.4 Hz, 1H), 4.58-4.41 (m, 3H), 3.96-3.83 (m, 2H), 3.51-3.43 (m, 1H), 3.10-3.09 (m, 1H), 3.00 (s, 3H), 1.38 (d, J = 6.8 Hz, 3H), 1.28 (d, J = 6.8 Hz, 3H). | 433.2 (M + 1)⁺ |
| 163 | 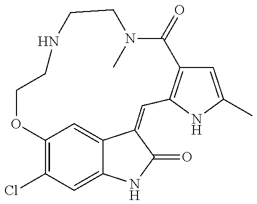 | 11.85-11.70 (m, 1H), 10.39 (s, 1H), 8.53 (s, 1H), 7.60 (s, 1H), 7.58 (s, 1H), 7.34-7.31 (m, 1H), 7.06 (s, 1H), 6.62- 6.41 (m, 1H), 4.40-4.31 (m, 2H), 3.51- 3.33 (m, 4H), 3.28-3.15 (m, 2H), 2.81 (s, 3H) | 387.1 (M + 1)⁺ |
| 165 | 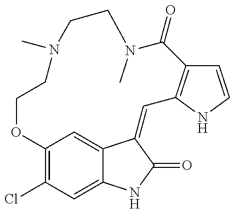 [SMILES: CN1CCOc2cc3c(cc2Cl)NC(=O)/C3=C\c2[nH]ccc2C(=O)N(C)CC1] | 11.69 (s, 1H), 10.63 (s, 1H), 8.45 (s, 1H), 7.45 (s, 1H), 7.35 (s, 1H), 6.84 (s, 1H), 6.43 (t, J = 2.4 Hz, 1H), 4.70 (t, J = 12.0 Hz, 1H), 4.35 (s, 2H), 3.03-2.95 (m, 1H), 2.93-2.89 (m, 4H), 2.76-2.71 (m, 1H), 2.71-2.63 (m, 1H), 2.35-2.28 (m, 1H), 2.23 (s, 3H) | 401.0 (M + 1)⁺ |
| 166 | 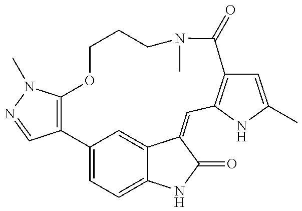 [SMILES: Cc1cc2c([nH]1)/C=C1\C(=O)Nc3ccc(cc31)-c1cnn(C)c1OCCCN(C)C2=O] | 11.55 (s, 1H), 10.76 (s, 1H), 7.93-7.75 (m, 2H), 7.39 (dd, J = 1.6, 8.0 Hz, 1H), 7.21 (s, 1H), 6.84 (d, J = 8.0 Hz, 1H), 6.20 (d, J = 2.0 Hz, 1H), 4.50 (d, J = 8.8 Hz, 1H), 4.34-4.25 (m, 2H), 3.71 (s, 3H), 2.78 (s, 3H), 2.66 (m, 1H), 2.38 (s, 3H), 2.17- 2.05 (m, 1H) | 418.1 (M + 1)⁺ |
| 167 | 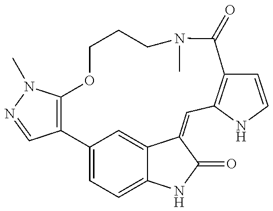 [SMILES: CN1CCCOc2c(cnn2C)-c2ccc3c(c2)/C(=C/c2[nH]ccc2C1=O)C(=O)N3] | 11.76 (s, 1H), 10.80 (s, 1H), 7.83-7.82 (m, 2H), 7.42-7.40 (m, 2H), 7.34-7.28 (m, 1H), 6.86 (d, 8.0 Hz, 1H), 6.41 (d, 8.0 Hz, 1H), 4.81 (d, J = 8.8 Hz, 1H), 4.50-4.27 (m, 2H), 3.63 (s, 3H), 3.33-3.32 (d, J = 4.8 Hz, 1H)), 2.78 (s, 1H), 2.70-2.66 (m, 1H), 2.49-2.41 (m, 1H). | 404.1 (M + 1)⁺ |
| 169 | 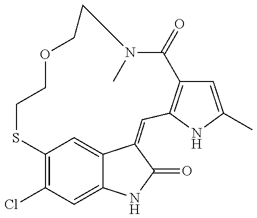 [SMILES: Cc1cc2c([nH]1)/C=C1\C(=O)Nc3cc(Cl)c(cc31)SCCOCCN(C)C2=O] | 12.02 (s, 1H), 10.99-10.77 (m, 1H), 7.61 (s, 1H), 7.37 (s, 1H), 6.93 (s, 1H), 6.26 (d, J = 2.0 Hz, 1H), 4.56-4.45 (m, 1H), 3.91-3.84 (m, 1H), 3.83-3.78 (m, 1H), 3.76-3.70 (m, 1H), 3.70-3.64 (m, 1H), 3.40-3.35 (m, 1H), 3.24-3.18 (m, 1H), 3.00-2.93 (m, 4H), 2.40 (s, 3H). | 418.1 (M + 1)⁺ |
| 170 | 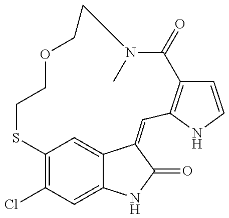 [SMILES: CN1CCOCCSc2cc3c(cc2Cl)NC(=O)/C3=C\c2[nH]ccc2C1=O] | 12.17 (s, 1H), 10.93 (s, 1H), 7.64 (s, 1H), 7.44 (s, 1H), 7.40 (t, J = 2.8 Hz, 1H), 6.94 (s, 1H), 6.45 (t, J = 2.4 Hz, 1H), 4.58-4.48 (m, 1H), 3.90-3.79 (m, 2H), 3.77-3.65 (m, 2H), 3.42-3.35 (m, 1H), 3.26-3.18 (m, 1H), 3.02-2.97 (m, 1H), 2.95 (s, 3H). | 404.1 (M + 1)⁺ |
| 171 | 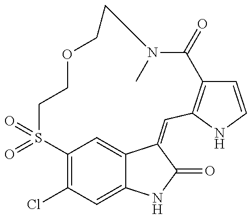 [SMILES: CN1CCOCCS(=O)(=O)c2cc3c(cc2Cl)NC(=O)/C3=C\c2[nH]ccc2C1=O] | 12.30-12.05 (m, 1H), 11.39-10.91 (m, 1H), 8.20 (s, 1H), 7.50 (s, 1H), 7.45 (t, J = 2.4 Hz, 1H), 7.11 (s, 1H), 6.49 (t, J = 2.0 Hz, 1H), 4.58-4.49 (m, 1H), 4.14 (br s, 1H), 4.09 (br d, J = 5.2 Hz, 2H), 3.93- 3.65 (m, 3H), 3.01-2.94 (m, 1H), 2.91 (s, 3H) | 436.0 (M + 1)⁺ |

### Preparation of [16a(17)Z]-19-chloro-5-methyl-6,7,9,10-tetrahydro-1H-12,14-(ethanediylidene)pyrazolo[4,3-i]pyrrolo[3,4-1][1,4,7]dioxazacyclopentadecine-4,16(5H,15H)-dione (159)

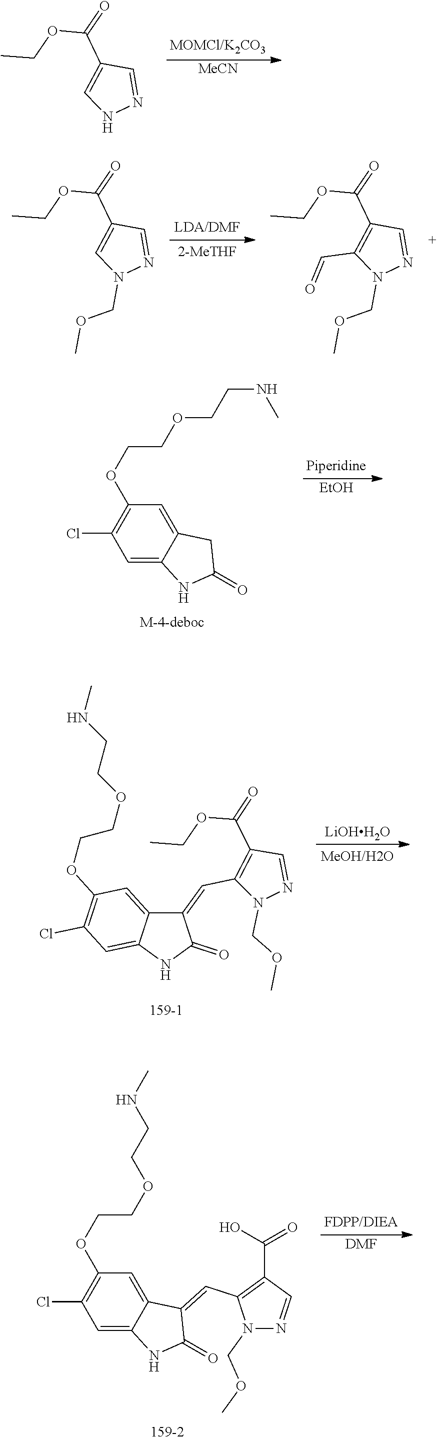

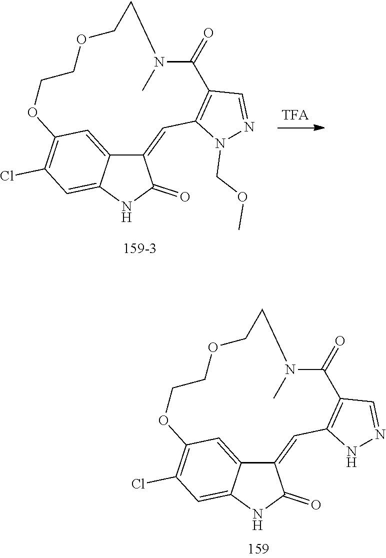 [SMILES: COCn1ncc2c1/C=C1\C(=O)Nc3cc(Cl)c(cc31)OCCOCCN(C)C2=O ; CN1CCOCCOc2cc3c(cc2Cl)NC(=O)/C3=C\c2[nH]ncc2C1=O]

Step 1. To the mixture of ethyl 1H-pyrazole-4-carboxylate (20.0 g, 142 mmol, 1.0 eq) and K₂CO₃ (39.4 g, 285 mmol, 2.0 eq) in MeCN (250 mL) at 0° C. was added MOMCl (18.1 g, 225 mmol, 1.5 eq). The mixture was heated to 40° C. and stirred for 2 hours. On completion, the mixture was quenched with water (30 mL) and concentrated in vacuum to afford a mixture (50 mL), which was diluted with brine (100 mL), and extracted with EtOAc (2*100 mL). The organic layer was dried over sodium sulfate, concentrated in vacuum to afford crude, which was purified by silica gel column (PE:EA=2:1) to afford ethyl 1-(methoxymethyl)pyrazole-4-carboxylate (17.1 g, 83 mmol, 59% yield) as colorless oil. ¹H NMR (400 MHz, DMSO-d₆) δ=8.51 (s, 1H), 7.94 (s, 1H), 5.42 (s, 2H), 4.22 (q, J=6.8 Hz, 2H), 3.25 (s, 3H), 1.26 (t, J=6.8 Hz, 3H).

Step 2. To a solution of DIPA (9.8 g, 97 mmol, 2.0 eq) in 2-MeTHF (90 mL) was added n-BuLi (2.5 M, 39.09 mL, 2.0 eq) at −70° C. The mixture was stirred at −70° C. for 25 minutes. The result LDA mixture was transferred to the solution of ethyl1-(methoxymethyl)pyrazole-4-carboxylate (9.0 g, 48.86 mmol, 1.0 eq) in 2-MeTHF (45 mL) at −70° C. with stirring for 5 minutes. To the mixture was added anhydrous DMF (35.72 g, 488 mmol, 10.0 eq) with stirring at −70° C. for another 1 hour. On completion, the mixture was quenched with Sat. NH₄Cl (300 mL), and extracted with EtOAc (300 mL). The organic layer was washed with brine (80 mL), dried over sodium sulfate, concentrated in vacuum to afford crude. The crude was purified by silica gel column (PE: EA=100:15) to afford ethyl 5-formyl-1-(methoxymethyl)pyrazole-4-carboxylate (4.0 g, 16.96 mmol, 34.72% yield) as colorless oil. ¹H NMR (400 MHz, DMSO-d₆) δ=10.33 (s, 1H), 8.10 (s, 1H), 5.69 (s, 2H), 4.32 (q, J=7.2 Hz, 2H), 1.98 (s, 2H), 1.32 (t, J=7.2 Hz, 3H).

Step 3. To a solution of ethyl 5-formyl-1-(methoxymethyl)pyrazole-4-carboxylate (90 mg, 0.424 mmol, 1.0 eq) in EtOH (22 mL) were added 6-chloro-5-[2-[2-(methylamino)ethoxy]ethoxy]indolin-2-one (M-4-deboc, 120.76 mg, 0.424 mmol, 1.0 eq) and piperidine (144 mg, 1.70 mmol, 4.0 eq). The mixture was stirred at 80° C. for 16 hours. On completion, the reaction mixture was concentrated in vacuum to afford crude. The crude was purified by silica gel column (DCM: MeOH=100:13) to afford 159a (150 mg, 0.288 mmol, 66% yield) as red solid. LCMS: m/z 479.3 (M+1)⁺.

Step 4. To the mixture of 159a (100 mg, 0.208 umol, 1.0 eq) in MeOH (8.0 mL) and H₂O (8.0 mL) was added LiOH—H₂O (105 mg, 2.51 mmol, 12.0 eq). The mixture was stirred at 20° C. for 16 hours. On completion, the mixture was concentrated in vacuum, re-dissolved in water (20.0 mL), adjusted to pH=6-7 with aq. HCl (1M), and then lyophilized. The residue was re-dissolved in DCM/MeOH (10:1), then filtered and concentrated in vacuum to 159b (120 mg, 0.186 mmol, 89.2% yield) as red solid. LCMS: m/z 451.2 (M+1)⁺.

Step 5. To a solution of 159b (100 mg, 0.221 mmol, 1.0 eq) and DIEA (86.0 mg, 0.665 mmol, 3.0 eq) in DMF (20.0 mL) was added FDPP (93.7 mg, 0.244 mmol, 1.1 eq). The mixture was stirred at 20° C. for 1 hour. On completion, the mixture was diluted with EtOAc (100 mL), and washed with brine (3*30 mL). The organic layer was concentrated in vacuum and purified by silica gel column (DCM:MeOH=100:4) to afford 159c (30.0 mg, 55.4 umol, 25.0% yield) as red solid. LCMS: m/z 433.2 (M+1)⁺.

Step 6. The mixture of 159c (20.0 mg, 46.2 umol, 1.0 eq) in TFA (1 mL) was stirred at 60° C. for 2 hours. On completion, the mixture was concentrated in vacuum, adjusted pH to neutral with Sat. NaHCO₃, and then lyophilized. The residue was purified by silica gel column (DCM: MeOH=100:5) to 159 (2.05 mg, 5.27 umol, 11.4% yield) as yellow solid. ¹H NMR (400 MHz, DMSO-d₆) δ=13.80-13.49 (m, 1H), 10.72-10.45 (m, 1H), 8.43-7.92 (m, 1H), 7.53-7.38 (m, 1H), 7.22-7.18 (m, 1H), 6.84 (d, J=7.2 Hz, 1H), 4.28-4.10 (m, 1H), 4.00-3.77 (m, 1H), 3.63 (d, J=11.2 Hz, 2H), 3.49 (d, J=3.2 Hz, 1H), 3.44 (dd, J=5.2, 7.2 Hz, 3H), 2.91-2.72 (m, 2H). LCMS: m/z 389.2 (M+1)⁺.

### Preparation of [16a(17)Z]-19-chloro-2,5,8-trimethyl-5,6,7,8,9,10-hexahydro-12,14-(ethanediylidene)dipyrrolo[3,2-i:3′,4′-l][1,4,7]oxadiazacyclopentadecine-4,16(1H,15H)-dione (164)

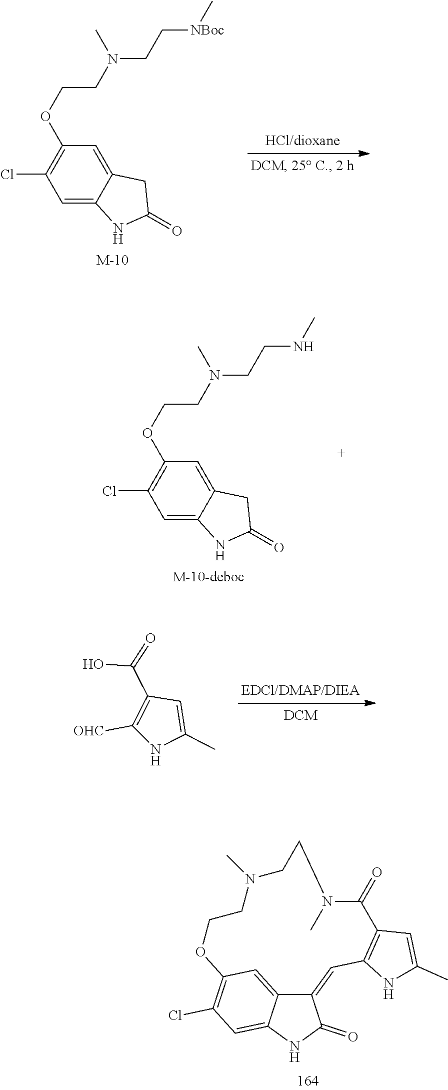

Step 1. To a solution of tert-butyl N-[2-[2-(6-chloro-2-oxo-indolin-5-yl)oxyethyl-methyl-amino]ethyl]-N-methyl-carbamate (M-10, 1.50 g, 3.77 mmol, 1 eq) in DCM (30 mL) was added HCl/dioxane (4 M, 18.8 mL, 20 eq). The mixture was stirred at 25° C. for 2 hr. On completion, the mixture was concentrated to provide M-10-deboc HCl salt which was used for the next step directly. LCMS: m/z 298.0 (M+1)⁺.

Step 2. To a solution of 2-formyl-5-methyl-1H-pyrrole-3-carboxylic acid (183 mg, 1.20 mmol, 1 eq) in DCM (15 mL) was added EDCI (458 mg, 2.39 mmol, 2 eq), DIEA (464 mg, 3.59 mmol, 625 uL, 3 eq) and DMAP (146 mg, 1.20 mmol, 1 eq). The mixture was stirred at 25° C. for 0.5 h. And then 6-chloro-5-[2-[methyl-[2-(methylamino)ethyl]amino]ethoxy]indolin-2-one (M-10-deboc HCl salt) (400 mg, 1.20 mmol, 1 eq) was added to the mixture. The mixture was stirred at 25° C. for 2 h. On completion, the mixture was concentrated to give a residue. The residue was purified by flash silica gel chromatography (12 g silica gel column, DCM in MeOH from 0% to 100%) to provide 164 (10.7 mg, 2.09% yield) as orange solid. ¹H NMR (400 MHz, DMSO-d₆) δ=11.48 (d, J=0.8 Hz, 1H), 10.61 (s, 1H), 8.41 (s, 1H), 7.39 (s, 1H), 6.83 (s, 1H), 6.22 (d, J=1.8 Hz, 1H), 4.75-4.62 (m, 1H), 4.40-4.28 (m, 2H), 3.31 (s, 3H), 2.82 (s, 4H), 2.67 (s, 1H), 2.39 (s, 3H), 2.21 (s, 3H). LCMS: m/z 415.1 (M+1)⁺.

### Preparation of [16a(17)Z]-2,5-dimethyl-4,16-dioxo-4,5,6,7,9,10,15,16-octahydro-1H-12,14-(ethanediylidene)dipyrrolo[3,2-i:3′,4′-l][1,4,7]dioxazacyclopentadecine-19-carbonitrile (168)

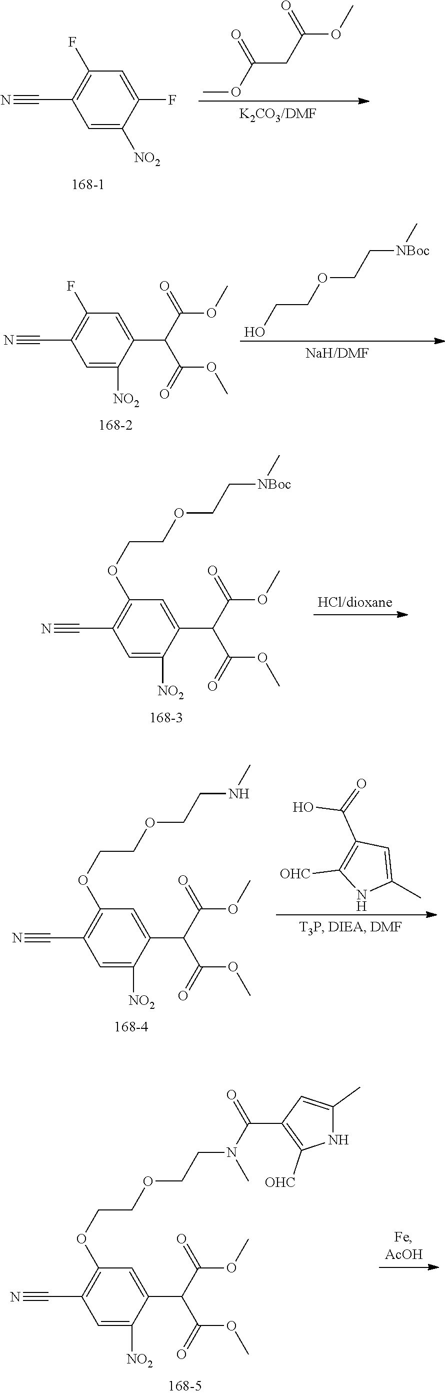

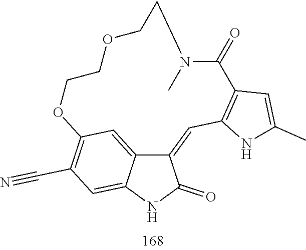 [SMILES: Cc1cc2c([nH]1)/C=C1\C(=O)Nc3cc(C#N)c(cc31)OCCOCCN(C)C2=O]

Step 1. To a solution of dimethyl propanedioate (4.11 g, 31.0 mmol, 3.57 mL, 1.2 eq) in DMF (80 mL) was added K₂CO₃ (4.28 g, 31.0 mmol, 1.2 eq) was added in small portions at 0° C. and stirred for 1.0 h followed by addition of 2,4-Difluoro-5-nitrobenzonitrile (4.77 g, 25.9 mmol, 1.0 eq) in portions and the mixture was stirred at 70° C. for 16 h. On completion, the mixture was poured into cold water (150 mL), extracted with EtOAc (250 mL). The organic layer was washed with brine (150 mL), dried over sodium sulfate, concentrated in vacuum to afford crude. The crude was purified by silica gel column to afford dimethyl2-(4-cyano-5-fluoro-2-nitrophenyl)propanedioate (168-2, 3.65 g, 12.3 mmol, 47.6% yield).

Step 2. To a solution of tert-butyl N-[2-(2-hydroxyethoxy)ethyl]-N-methyl-carbamate (3.00 g, 13.7 mmol, 1 eq) in DMF (40 mL) was added NaH (1.09 g, 27.4 mmol, 60% purity, 2 eq) and the mixture was stirred at 0° C. followed by addition of dimethyl2-(4-cyano-5-fluoro-2-nitrophenyl)propanedioate (3.65 g, 12.3 mmol, 0.9 eq). The mixture was stirred at 20° C. for 30 min and heated to 80° C. for 2 hours. The reaction mixture was quenched by addition of H₂O (80 mL) and extracted with EtOAc (100 mL×3). The combined organic layers were washed with brine (40 mL×3), dried over anhydrous Na₂SO₄, filtered and dried. The residue was purified by column chromatography (SiO₂, Petroleum ether/Ethyl acetate=10/1 to 4/1) to give dimethyl 2-[5-[2-[2-[tert-butoxycarbonyl(methyl)amino]ethoxy]ethoxy]-4-cyano-2-nitro-phenyl]propanedioate (168-3, 850 mg, 1.68 mmol, 12.3% yield) as brown oil. LCMS: 518.1 (M+Na)⁺.

Step 3. The mixture of 168-3 (150 mg, 0.302 mmol, 1 eq) in TFA (0.50 mL) and DCM (1 mL) was stirred at 20° C. for 2 hours. The reaction mixture was concentrated under reduced pressure to give compound 168-4 (80 mg, 0.184 mmol, 60.8% yield) as brown oil. ¹H NMR (400 MHz, CDCl₃) δ=8.67-8.40 (m, 1H), 8.32 (s, 0.5H), 8.01 (s,0.5H), 7.19 (s, 1H), 5.48 (s, 1H), 4.72 (s, 4H), 4.39 (s, 1H), 3.93-3.77 (m, 3H), 3.77-3.74 (m, 3H), 3.29-3.17 (m, 1H), 3.00-2.86 (m, 3H), 2.75 (s, 2H). LCMS: 396.1 (M+H)⁺.

Step 4. A mixture of 2-formyl-5-methyl-1H-pyrrole-3-carboxylic acid (85.2 mg, 0.556 mmol, 1 eq), DIEA (287 mg, 2.23 mmol, 4 eq), T₃P (265 mg, 0.835 mmol, 1.5 eq) and 168-4 (220 mg, 0.556 mmol, 1 eq) in DMF (2 mL) was stirred at 20° C. for 2 hours and quenched by addition of H₂O (10 mL) and extracted with EtOAc (10 mL×3). The combined organic layers were washed with brine (10 mL×3), dried over anhydrous Na₂SO₄, filtered and dried. The residue was purified by prep-TLC (SiO₂, DCM: MeOH=10:1) to give compound 168-5 (140 mg, 0.251 mmol, 45.1% yield) as yellow oil. LCMS: m/z 531.1 (M+1)⁺.

Step 5. To a mixture of 168-5 (110 mg, 0.207 mmol, 1 eq) in AcOH (2 mL) was added Fe (57.9 mg, 1.04 mmol, 5 eq). The reaction mixture was stirred at 100° C. for 4 hours. The mixture was filtered and concentrated under reduced pressure. The residue was purified by prep-TLC (DCM: MeOH=10:1) to give compound 168 (16.2 mg, 0.0392 mmol, 18.9% yield) as orange solid.

¹H NMR (400 MHz, DMSO-d₆) δ=11.48 (s, 1H), 10.81 (s, 1H), 8.01 (s, 1H), 7.49 (s, 1H), 7.06 (s, 1H), 6.31 (s, 1H), 4.61-4.42 (m, 3H), 3.93-3.75 (m, 3H), 3.69 (s, 1H), 3.01 (dd, J=4.8, 14.4 Hz, 1H), 2.82 (s, 3H), 2.42 (s, 3H); LCMS: m/z 393.2 (M+1)⁺.

### Preparation of [3a(4)Z]-6,9,12,14-tetramethyl-9,10,11,12,13,14-hexahydro-17,1-(azenometheno)pyrazolo[4,3-m]dipyrrolo[3,2-f:3′,4′-i][1,4]diazacyclopentadecine-3,8(2H,5H)-dione (172)

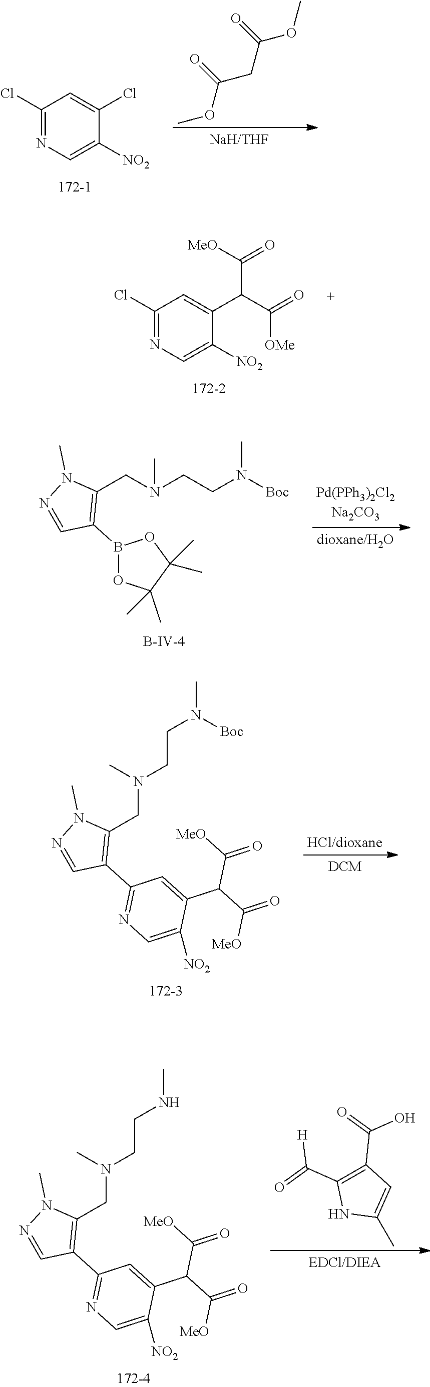

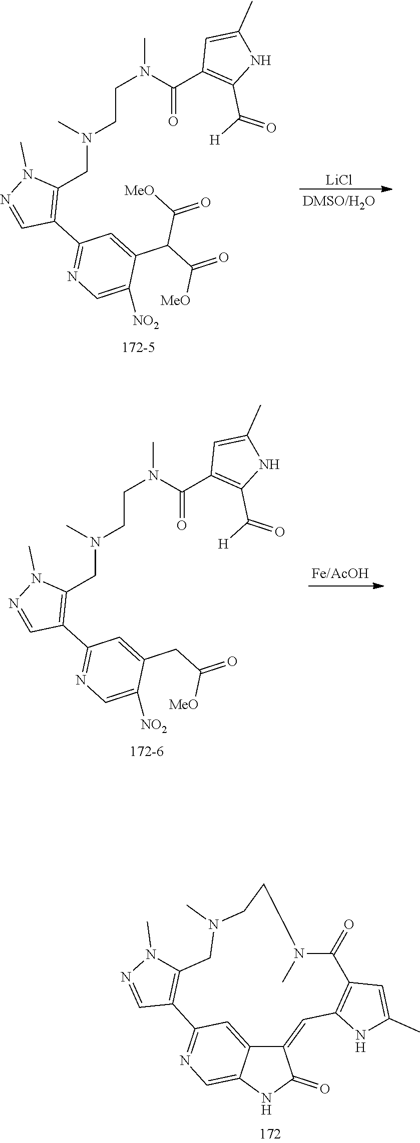 [SMILES: COC(=O)C(C(=O)OC)c1cc(-c2cnn(C)c2CN(C)CCN(C)C(=O)c2cc(C)[nH]c2C=O)ncc1[N+](=O)[O-] ; "LiCl" ; COC(=O)Cc1cc(-c2cnn(C)c2CN(C)CCN(C)C(=O)c2cc(C)[nH]c2C=O)ncc1[N+](=O)[O-] ; Cc1cc2c([nH]1)/C=C1\C(=O)Nc3cnc(cc31)-c1cnn(C)c1CN(C)CCN(C)C2=O]

Step 1. To a solution of dimethyl propanedioate (4.11 g, 31.0 mmol, 1.2 eq) in THF (80 mL) was added NaH (1.24 g, 31.09 mmol, 60% purity, 1.2 eq) in small portions at 0° C. and the mixture was stirred for 1.0 h followed by addition of 2,4-dichloro-5-nitro-pyridine (5.0 g, 25.9 mmol, 1.0 eq) in portions. The mixture was stirred at 70° C. for 16 h. On completion, the mixture was poured into cold water (150 mL), extracted with EtOAc (250 mL). The organic layer was washed with brine (150 mL), dried over sodium sulfate, concentrated in vacuum. The residue was purified by silica gel column (PE: EA=100: 0-100:30) to afford 172-2 (3.6 g, 11.2 mmol, 43.3% yield) as yellow oil. ¹H NMR (400 MHz, CDCl₃) δ=9.08 (s, 1H), 7.52 (s, 1H), 5.37 (s, 1H), 3.82 (s, 6H). LCMS: m/z 289.1 (M+1)⁺.

Step 2. The mixture of 172-2 (600 mg, 2.08 mmol, 1.0 eq), B-IV-4 (1.41 g, 2.08 mmol, 60% purity, 1.0 eq) and Na₂CO₃ (2 M, 3.12 mL, 3.0 eq) in dioxane (8.0 mL), Pd(dppf)Cl₂ (152 mg, 0.207 mmol, 0.1 eq) was added. The mixture was stirred at 90° C. for 2 h under nitrogen atmosphere. On completion, the mixture was concentrated in vacuum and purified by silica gel column (DCM: MeOH=100:2) to afford 172-3 (800 mg, 1.50 mmol, 71.9% yield) as red-brown gum. LCMS: m/z 535.4 (M+1)⁺.

Step 3. To a solution of 172-3 (350 mg, 654 umol, 1.0 eq) in DCM (10 mL) was added HCl/dioxane (4 M, 1.64 mL, 10 eq). The mixture was stirred at 20° C. for 1 h. On completion, the mixture was concentrated in vacuum to afford 172-4 (300 mg, 0.621 mmol, 94.9% yield) as off-white solid. LCMS: m/z 435.3 (M+1)⁺.

Step 4. To a solution of 2-formyl-5-methyl-1H-pyrrole-3-carboxylic acid (102 mg, 0.667 mmol, 1.0 eq), DIEA (258 mg, 2.00 mmol, 3.0 eq) and DMAP (8.15 mg, 0.0667 mmol, 0.1 eq) in DCM (10 mL) was added EDCI (191 mg, 1.00 mmol, 1.5 eq) at 20° C. and the mixture was stirred for 0.5 h followed by addition of 172-4 (290 mg, 0.667 mmol, 1.0 eq). The mixture was stirred for 0.5 h. On completion, the mixture was concentrated in vacuum to afford and purified on silica gel column (DCM: MeOH=30:1) to afford 172-5 (300 mg, 0.474 mmol, 71.0% yield)

LCMS: m/z 570.7 (M+1)⁺.

Step 5. To a solution of 172-5 (250 mg, 0.438 mmol, 1.0 eq) in DMSO (5.0 mL) and H₂O (1.0 mL) was LiCl (55.8 mg, 1.32 mmol, 3.0 eq). The mixture was stirred at 100° C. for 7 h. On completion, the mixture was diluted with EtOAc (100 mL), and washed with brine (2*40 mL). The organic layer was dried over sodium sulfate, concentrated in vacuum, and purified by silica gel column (DCM: MeOH=20:1) to afford 172-6 (180 mg, 0.334 mmol, 76.1% yield) as brown gum. LCMS: m/z 512.1 (M+1)⁺.

Step 6. To a solution of 172-6 (150 mg, 0.293 mmol, 1.0 eq) in AcOH (40.0 mL) was added Fe (163 mg, 2.93 mmol, 10.0 eq). The mixture was stirred at 90° C. for 1 h. The mixture was filtered and the filtrate was concentrated in vacuum. The residue was re-dissolved in MeOH (20.0 mL), and sat. NaHCO₃ (20.0 mL) was added dropwise and stirred at 20° C. for 2 h. The mixture was concentrated in vacuum to afford gum. The gum was re-dissolved in DCM/MeOH (ratio=10:1, 100 mL), filtered and the filtrate was concentrated in vacuum to afford crude. The crude was purified by silica gel column (DCM: MeOH=100:5) to afford 172 (7.78 mg, 0.165 mmol, 5.66% yield) as orange powder.

173 and 174 were parepared using similar procedures as 172 starting with 2,6-dichloro-3-nitro-pyridine.

| Ex | | | |
|---|---|---|---|
| # | Structure | ¹H NMR (400 MHz, DMSO-d₆) δ ppm | MS m/z |
| 172 | 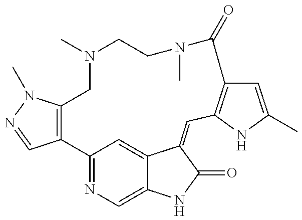 [SMILES: Cc1cc2c([nH]1)/C=C1\C(=O)Nc3cnc(cc31)-c1cnn(C)c1CN(C)CCN(C)C2=O] | 11.45 (s, 1H), 10.85 (s, 1H), 8.70-8.54 (m, 1H), 8.09 (s, 1H), 7.90 (s, 1H), 7.60 (s, 1H), 6.31 (d, J = 1.6 Hz, 1H), 4.57 (m, 1H), 3.92 (s, 3H), 3.53-3.46 (m, 1H), 3.20-3.04 (m, 2H), 2.96 (s, 3H), 2.42 (s, 3H), 2.35-2.28 (m, 1H), 2.21 (s, 3H), 2.17 (m, 1H). | 432.2 (M + 1)⁺ |
| 173 | 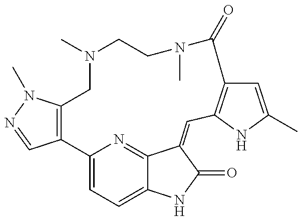 [SMILES: Cc1cc2c([nH]1)/C=C1\C(=O)Nc3ccc(nc31)-c1cnn(C)c1CN(C)CCN(C)C2=O] | 12.42 (s, 1H), 10.88 (s, 1H), 8.00 (s, 1H), 7.92 (s, 1H), 7.47 (d, J = 8.4 Hz, 1H), 7.19 (d, J = 8.4 Hz, 1H), 6.27 (d, J = 2.0 Hz, 1H), 4.43-4.31 (m, 2H), 3.86 (s, 3H), 3.33-3.27 (m, 2H), 3.06-3.01 (m, 2H), 2.99 (s, 3H), 2.41 (s, 3H), 2.15 (s, 3H). | 432.2 (M + 1)⁺ |
| 174 | 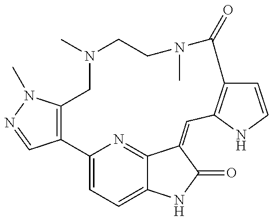 [SMILES: CN1CCN(C)C(=O)c2cc[nH]c2/C=C2\C(=O)Nc3ccc(nc32)-c2cnn(C)c2C1] | 12.52 (s, 1H), 10.91 (s, 1H), 8.00 (s, 1H), 7.97 (s, 1H), 7.49 (d, J = 8.4 Hz, 1H), 7.44 (t, J = 2.6 Hz, 1H), 7.19 (d, J = 8.3 Hz, 1H), 6.46 (t, J = 2.4 Hz, 1H), 4.46- 4.29 (m, 2H), 3.85 (s, 3H), 3.34-3.25 (m, 2H), 3.06-3.02 (m, 2H), 2.98 (s, 3H), 2.14 (s, 3H). | 418.3 (M + 1)⁺ |

### Screen Assays

### Biochemical Assay

Kinase binding assays were performed at Eurofins/DiscoveRx using the general KINOMEscan Protocol (Fabian, M. A. et al., “A small molecule-kinase interaction map for clinical kinase inhibitors,” Nat. Biotechnol. 2005, 23(3):329-36). For most assays, kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce nonspecific binding. Binding reactions were assembled by combining kinases, liganded affinity beads, and test compounds in 1×binding buffer (20% SeaBlock, 0.17×PBS, 0.05% Tween 20, 6 mM DTT). All reactions were performed in polystyrene 96-well plates in a final volume of 0.135 mL. The assay plates were incubated at room temperature with shaking for 1 hour and the affinity beads were washed with washbuffer (1×PBS, 0.05% Tween 20). The beads were then re-suspended in elution buffer (1× PBS, 0.05% Tween 20, 0.5 μM non-biotinylated affinity ligand) and incubated at room temperature with shaking for 30 minutes. The kinase concentration in the eluates was measured by qPCR. Results for compounds tested in this assay at a given concentration are reported as “% Ctrl”, where lower numbers indicate stronger binding in the matrix.

% Ctrl Calculation:

(test compound signal-positive control signal)/(negative control signal-positive control signal) X100

TABLE 1

| | Ex.71 | Ex.111 | Ex. 141 | Ex.22 | Ex.39 | Ex. 41 | Ex.91 | Ex.123 |
|---|---|---|---|---|---|---|---|---|
| | (1 μM, | (1 μM, | (1 μM, | (1 μM, | (1 μM, | (1 μM, | (1 μM, | (1 μM, |
| Target | % Ctrl) | % Ctrl) | % Ctrl) | % Ctrl) | % Ctrl) | % Ctrl) | % Ctrl) | % Ctrl) |
| ABL1(E255K)- | 85 | 42 | 100 | 67 | 72 | 49 | 14 | 92 |
| phosphorylated | | | | | | | | |
| ABL1(F317I)- | 100 | 89 | 100 | 88 | 100 | 54 | 20 | 100 |
| nonphosphorylated | | | | | | | | |
| ABL1(F317I)- | 100 | 100 | 100 | 100 | 64 | 75 | 39 | 100 |
| phosphorylated | | | | | | | | |
| ABL1(F317L)- | 100 | 87 | 100 | 82 | 98 | 26 | 12 | 100 |
| nonphosphorylated | | | | | | | | |
| ABL1(F317L)- | 100 | 100 | 100 | 100 | 60 | 53 | 20 | 100 |
| phosphorylated | | | | | | | | |
| ABL1(H396P)- | 100 | 62 | 100 | 90 | 74 | 6.1 | 0.55 | 100 |
| nonphosphorylated | | | | | | | | |
| ABL1(H396P)- | 98 | 61 | 100 | 77 | 82 | 5 | 15 | 100 |
| phosphorylated | | | | | | | | |
| ABL1(M351T)- | 100 | 98 | 100 | 100 | 58 | 53 | 8.5 | 100 |
| phosphorylated | | | | | | | | |
| ABL1(Q252H)- | 97 | 75 | 100 | 87 | 84 | 9 | 2.8 | 92 |
| nonphosphorylated | | | | | | | | |
| ABL1(Q252H)- | 100 | 57 | 100 | 74 | 93 | 63 | 11 | 100 |
| phosphorylated | | | | | | | | |
| ABL1(T315I)- | 100 | 90 | 100 | 97 | 95 | 16 | 4.5 | 100 |
| nonphosphorylated | | | | | | | | |
| ABL1(T315I)- | 100 | 64 | 100 | 100 | 26 | 19 | 5.8 | 100 |
| phosphorylated | | | | | | | | |
| ABL1(Y253F)- | 91 | 58 | 100 | 68 | 98 | 49 | 7.8 | 100 |
| phosphorylated | | | | | | | | |
| ABL1- | 90 | 71 | 99 | 71 | 73 | 7.1 | 0.75 | 83 |
| nonphosphorylated | | | | | | | | |
| ABL1-phosphorylated | 93 | 59 | 100 | 69 | 78 | 56 | 15 | 100 |
| ABL2 | 100 | 100 | 100 | 95 | 89 | 77 | 32 | 100 |
| EGFR | 95 | 74 | 100 | 64 | 77 | 62 | 99 | 98 |
| EGFR(E746-A750del) | 100 | 92 | 100 | 97 | 69 | 36 | 93 | 100 |
| EGFR(G719C) | 71 | 73 | 100 | 93 | 70 | 88 | 92 | 90 |
| EGFR(G719S) | 76 | 77 | 100 | 95 | 77 | 86 | 87 | 94 |
| EGFR(L747-E749del, | 92 | 83 | 100 | 73 | 68 | 87 | 100 | 9 |
| A750P) | | | | | | | | |
| EGFR(L747-S752del, | 100 | 100 | 100 | 95 | 66 | 78 | 94 | 100 |
| P753S) | | | | | | | | |
| EGFR(L747- | 67 | 59 | 100 | 81 | 60 | 77 | 68 | 74 |
| T751del, Sins) | | | | | | | | |
| EGFR(L858R) | 9 | 78 | 100 | 67 | 64 | 93 | 100 | 94 |
| EGFR(L858R, T790M) | 100 | 97 | 100 | 97 | 90 | 17 | 58 | 92 |
| EGFR(L861Q) | 80 | 64 | 100 | 87 | 68 | 91 | 66 | 100 |
| EGFR(S752-1759del) | 74 | 54 | 100 | 72 | 80 | 89 | 63 | 93 |
| EGFR(T790M) | 100 | 68 | 100 | 57 | 99 | 36 | 82 | 100 |
| FLT1 | 98 | 46 | 100 | 100 | 95 | 81 | 83 | 96 |
| FLT3 | 93 | 45 | 100 | 81 | 100 | 5.5 | 11 | 87 |
| FLT3(D835H) | 99 | 20 | 100 | 89 | 85 | 5.2 | 1.2 | 88 |
| FLT3(D835V) | 100 | 1.5 | 100 | 35 | 35 | 0.25 | 0.05 | 91 |
| FLT3(D835Y) | 94 | 9.1 | 100 | 93 | 96 | 8.8 | 0.25 | 72 |
| FLT3(ITD) | 91 | 6.9 | 100 | 100 | 89 | 13 | 2.1 | 93 |
| FLT3(ITD, D835V) | 100 | 0.45 | 100 | 71 | 17 | 0.8 | 0 | 100 |
| FLT3(ITD, F691L) | 100 | 5.8 | 100 | 100 | 27 | 0.35 | 0 | 39 |
| FLT3(K663Q) | 84 | 44 | 100 | 100 | 100 | 17 | 63 | 77 |
| FLT3(N841I) | 75 | 14 | 100 | 65 | 83 | 0 | 0 | 95 |
| FLT3(R834Q) | 100 | 39 | 100 | 97 | 93 | 15 | 29 | 100 |
| FLT3-autoinhibited | 100 | 95 | 100 | 100 | 85 | 50 | 38 | 100 |
| FLT4 | 95 | 74 | 100 | 92 | 98 | 62 | 9.9 | 94 |
| KIT | 100 | 99 | 100 | 100 | 90 | 58 | 82 | 97 |
| KIT(A829P) | 100 | 100 | 100 | 100 | 99 | 45 | 32 | 100 |
| KIT(D816H) | 100 | 7 | 100 | 100 | 83 | 38 | 41 | 100 |
| KIT(D816V) | 100 | 76 | 100 | 96 | 91 | 38 | 20 | 100 |
| KIT(L576P) | 100 | 98 | 100 | 84 | 100 | 40 | 14 | 97 |
| KIT(V559D) | 100 | 97 | 100 | 96 | 99 | 64 | 62 | 99 |
| KIT(V559D, T670I) | 100 | 96 | 100 | 100 | 85 | 30 | 34 | 97 |
| KIT(V559D, V654A) | 100 | 62 | 100 | 100 | 91 | 93 | 8 | 79 |
| KIT-autoinhibited | 100 | 100 | 92 | 100 | 86 | 78 | 73 | 97 |
| PDGFRA | 100 | 95 | 95 | 100 | 89 | 19 | 52 | 100 |
| PDGFRB | 98 | 88 | 100 | 100 | 91 | 5.5 | 55 | 94 |
| RET | 96 | 94 | 100 | 81 | 85 | 34 | 4.3 | 91 |
| RET(M918T) | 91 | 76 | 100 | 92 | 97 | 45 | 7 | 89 |
| RET(V804L) | 95 | 71 | 100 | 98 | 87 | 17 | 3.8 | 89 |
| RET(V804M) | 89 | 51 | 100 | 93 | 94 | 19 | 4 | 99 |

TABLE 2

| | Ex. 124 | Ex. 125 | Ex. 126 | Ex. 127 | Ex. 128 | Ex. 129 | Ex. 131 |
|---|---|---|---|---|---|---|---|
| | (1 μM, | (1 μM, | (1 μM, | (1 μM, | (1 μM, | (1 μM, | (1 μM, |
| Target | % Ctrl) | % Ctrl) | % Ctrl) | % Ctrl) | % Ctrl) | % Ctrl) | % Ctrl) |
| ABL1(E255K)- | 0 | 0.05 | 0.55 | 0.1 | 25 | 89 | 0 |
| phosphorylated | | | | | | | |
| ABL1(F317I)- | 0 | 0 | 0 | 2.6 | 100 | 94 | 0 |
| nonphosphorylated | | | | | | | |
| ABL1(F317I)- | 1.5 | 0.75 | 0.3 | 5.5 | 55 | 100 | 3.5 |
| phosphorylated | | | | | | | |
| ABL1(F317L)- | 0 | 0 | 1.7 | 4.1 | 85 | 93 | 0 |
| nonphosphorylated | | | | | | | |
| ABL1(F317L)- | 0.2 | 0.55 | 0 | 0.8 | 58 | 79 | 1.3 |
| phosphorylated | | | | | | | |
| ABL1(H396P)- | 0.5 | 0 | 0 | 0 | 28 | 87 | 0 |
| nonphosphorylated | | | | | | | |
| ABL1(H396P)- | 0 | 0.1 | 0.1 | 0 | 51 | 93 | 0 |
| phosphorylated | | | | | | | |
| ABL1(M351T)- | 0.05 | 0.1 | 0.05 | 0.2 | 39 | 83 | 0.2 |
| phosphorylated | | | | | | | |
| ABL1(Q252H)- | 0 | 0 | 1.7 | 0 | 57 | 87 | 0 |
| nonphosphorylated | | | | | | | |
| ABL1(Q252H)- | 0 | 0.05 | 0.75 | 0.05 | 42 | 86 | 0.2 |
| phosphorylated | | | | | | | |
| ABL1(T315I)- | 0 | 0 | 0 | 0 | 20 | 92 | 0 |
| nonphosphorylated | | | | | | | |
| ABL1(T315I)- | 0.1 | 0.1 | 0 | 0.65 | 4.2 | 91 | 1.1 |
| phosphorylated | | | | | | | |
| ABL1(Y253F)- | 0 | 0 | 0.25 | 0 | 49 | 98 | 0 |
| phosphorylated | | | | | | | |
| ABL1- | 0 | 0 | 0 | 0.05 | 60 | 84 | 0 |
| nonphosphorylated | | | | | | | |
| ABL1-phosphorylated | 0 | 0 | 0.1 | 0 | 51 | 91 | 0 |
| ABL2 | 2.4 | 2.2 | 0.9 | 6.9 | 91 | 96 | 1.8 |
| EGFR | 50 | 61 | 23 | 42 | 91 | 98 | 52 |
| EGFR(E746-A750del) | 15 | 20 | 3.9 | 13 | 86 | 100 | 11 |
| EGFR(G719C) | 87 | 8 | 86 | 90 | 97 | 100 | 93 |
| EGFR(G719S) | 97 | 88 | 57 | 72 | 97 | 97 | 80 |
| EGFR(L747-E749del, | 25 | 22 | 1.8 | 26 | 85 | 88 | 31 |
| A750P) | | | | | | | |
| EGFR(L747-S752del, | 37 | 48 | 14 | 40 | 93 | 98 | 50 |
| P753S) | | | | | | | |
| EGFR(L747- | 57 | 54 | 27 | 43 | 94 | 85 | 59 |
| T751del, Sins) | | | | | | | |
| EGFR(L858R) | 58 | 48 | 31 | 47 | 88 | 100 | 56 |
| EGFR(L858R, T790M) | 1.7 | 1.2 | 0.15 | 2.3 | 48 | 98 | 35 |
| EGFR(L861Q) | 81 | 77 | 50 | 73 | 100 | 81 | 78 |
| EGFR(S752-I759del) | 68 | 73 | 32 | 57 | 98 | 78 | 70 |
| EGFR(T790M) | 8.5 | 11 | 1.6 | 9.2 | 75 | 100 | 61 |
| FLT1 | 13 | 11 | 0.65 | 54 | 85 | 99 | 4 |
| FLT3 | 0.15 | 0.45 | 0.2 | 4.6 | 66 | 100 | 0.6 |
| FLT3(D835H) | 0.05 | 0.05 | 0.05 | 0.15 | 23 | 44 | 0 |
| FLT3(D835V) | 0 | 0.25 | 0 | 0.15 | 1.7 | 17 | 0 |
| FLT3(D835Y) | 5.6 | 0 | 1.4 | 1.4 | 22 | 9.1 | 2.3 |
| FLT3(ITD) | 0 | 0 | 0.4 | 2.2 | 43 | 32 | 0.85 |
| FLT3(ITD, D835V) | 0 | 0 | 0 | 0 | 5.8 | 4.3 | 0 |
| FLT3(ITD, F691L) | 0.65 | 0 | 2.5 | 6.2 | 10 | 40 | 0.6 |
| FLT3(K663Q) | 13 | 1.4 | 4.2 | 5.3 | 77 | 100 | 7.4 |
| FLT3(N841I) | 0 | 0 | 0 | 0.35 | 36 | 68 | 0 |
| FLT3(R834Q) | 0 | 0.3 | 0 | 13 | 68 | 84 | 6.6 |
| FLT3-autoinhibited | 0.35 | 0.15 | 0.25 | 9.9 | 89 | 98 | 5.1 |
| FLT4 | 0.15 | 0.11 | 0 | 2.1 | 45 | 83 | 0 |
| KIT | 45 | 42 | 6.3 | 89 | 98 | 100 | 1.1 |
| KIT(A829P) | 4.3 | 2.6 | 0 | 60 | 86 | 100 | 4 |
| KIT(D816H) | 0.4 | 0.65 | 0 | 60 | 87 | 96 | 6.2 |
| KIT(D816V) | 0.7 | 0.5 | 0.3 | 48 | 84 | 93 | 1.2 |
| KIT(L576P) | 4.2 | 5.9 | 0 | 73 | 100 | 98 | 0 |
| KIT(V559D) | 36 | 35 | 1.4 | 82 | 93 | 98 | 0.15 |
| KIT(V559D, T670I) | 24 | 27 | 3.6 | 62 | 71 | 100 | 0.05 |
| KIT(V559D, V654A) | 7.7 | 6.7 | 0.4 | 89 | 100 | 98 | 12 |
| KIT-autoinhibited | 93 | 95 | 84 | 80 | 97 | 78 | 13 |
| PDGFRA | 7.9 | 7.7 | 1.2 | 47 | 89 | 100 | 7.7 |
| PDGFRB | 0.35 | 0.4 | 0 | 28 | 85 | 100 | 1.4 |
| RET | 0 | 0 | 0 | 2 | 83 | 100 | 0 |
| RET(M918T) | 0 | 0 | 0 | 2.1 | 93 | 100 | 0 |
| RET(V804L) | 0 | 0 | 0 | 0.55 | 56 | 96 | 0 |
| RET(V804M) | 0.05 | 0 | 0 | 1.6 | 55 | 94 | 0.15 |

TABLE 3

| | Ex. 43 | Ex. 92 | Ex. 133 | Ex. 134 | Ex. 135 | Ex. 136 | Ex. 137 |
|---|---|---|---|---|---|---|---|
| | (1 μM, | (1 μM, | (1 μM, | (1 μM, | (1 μM, | (1 μM, | (1 μM, |
| Target | % Ctrl) | % Ctrl) | % Ctrl) | % Ctrl) | % Ctrl) | % Ctrl) | % Ctrl) |
| ABL1(E255K)- | 84 | 22 | 70 | 50 | 56 | 36 | 64 |
| phosphorylated | | | | | | | |
| ABL1(F317I)- | 68 | 22 | 93 | 84 | 96 | 78 | 95 |
| nonphosphorylated | | | | | | | |
| ABL1(F317I)- | 94 | 89 | 89 | 70 | 84 | 66 | 83 |
| phosphorylated | | | | | | | |
| ABL1(F317L)- | 63 | 6.5 | 77 | 62 | 78 | 59 | 76 |
| nonphosphorylated | | | | | | | |
| ABL1(F317L)- | 90 | 76 | 88 | 57 | 64 | 44 | 67 |
| phosphorylated | | | | | | | |
| ABL1(H396P)- | 64 | 0.3 | 49 | 25 | 29 | 11 | 38 |
| nonphosphorylated | | | | | | | |
| ABL1(H396P)- | 89 | 46 | 67 | 35 | 54 | 22 | 65 |
| phosphorylated | | | | | | | |
| ABL1(M351T)- | 78 | 54 | 58 | 25 | 48 | 17 | 51 |
| phosphorylated | | | | | | | |
| ABL1(Q252H)- | 55 | 0 | 61 | 32 | 43 | 24 | 48 |
| nonphosphorylated | | | | | | | |
| ABL1(Q252H)- | 97 | 34 | 49 | 18 | 47 | 14 | 51 |
| phosphorylated | | | | | | | |
| ABL1(T315I)- | 68 | 12 | 94 | 84 | 94 | 77 | 81 |
| nonphosphorylated | | | | | | | |
| ABL1(T315I)- | 75 | 66 | 78 | 46 | 72 | 37 | 69 |
| phosphorylated | | | | | | | |
| ABL1(Y253F)- | 80 | 54 | 57 | 32 | 55 | 16 | 60 |
| phosphorylated | | | | | | | |
| ABL1- | 53 | 0 | 65 | 46 | 33 | 36 | 43 |
| nonphosphorylated | | | | | | | |
| ABL1-phosphorylated | 90 | 25 | 68 | 38 | 58 | 25 | 61 |
| ABL2 | 100 | 43 | 96 | 77 | 85 | 77 | 99 |
| EGFR | 61 | 98 | 99 | 96 | 100 | 98 | 100 |
| EGFR(E746-A750del) | 69 | 100 | 100 | 89 | 99 | 76 | 98 |
| EGFR(G719C) | 92 | 100 | 92 | 89 | 82 | 90 | 100 |
| EGFR(G719S) | 92 | 98 | 99 | 92 | 92 | 92 | 98 |
| EGFR(L747-E749del, | 89 | 91 | 100 | 83 | 82 | 80 | 100 |
| A750P) | | | | | | | |
| EGFR(L747-S752del, | 86 | 55 | 100 | 87 | 80 | 82 | 100 |
| P753S) | | | | | | | |
| EGFR(L747- | 85 | 98 | 100 | 99 | 98 | 94 | 100 |
| T751del, Sins) | | | | | | | |
| EGFR(L858R) | 97 | 98 | 95 | 89 | 93 | 90 | 99 |
| EGFR(L858R, T790M) | 98 | 100 | 95 | 92 | 93 | 93 | 93 |
| EGFR(L861Q) | 90 | 100 | 97 | 95 | 86 | 95 | 100 |
| EGFR(S752-I759del) | 91 | 90 | 90 | 88 | 92 | 95 | 98 |
| EGFR(T790M) | 100 | 100 | 98 | 92 | 94 | 96 | 94 |
| FLT1 | 96 | 90 | 89 | 97 | 85 | 97 | 88 |
| FLT3 | 99 | 77 | 48 | 48 | 57 | 36 | 55 |
| FLT3(D835H) | 93 | 32 | 39 | 35 | 37 | 32 | 53 |
| FLT3(D835V) | 27 | 3.7 | 2.3 | 3.9 | 6 | 2 | 8.5 |
| FLT3(D835Y) | 86 | 28 | 3.1 | 10 | 13 | 2.6 | 14 |
| FLT3(ITD) | 100 | 45 | 26 | 35 | 52 | 23 | 44 |
| FLT3(ITD, D835V) | 38 | 8.3 | 2.8 | 3.2 | 11 | 2.8 | 9.1 |
| FLT3(ITD, F691L) | 47 | 9.2 | 5.6 | 6.1 | 25 | 5.7 | 29 |
| FLT3(K663Q) | 72 | 57 | 77 | 73 | 66 | 64 | 69 |
| FLT3(N841I) | 98 | 20 | 19 | 26 | 41 | 14 | 49 |
| FLT3(R834Q) | 85 | 75 | 49 | 59 | 63 | 50 | 65 |
| FLT3-autoinhibited | 77 | 96 | 95 | 95 | 89 | 97 | 77 |
| FLT4 | 91 | 75 | 98 | 90 | 100 | 100 | 95 |
| KIT | 100 | 91 | 65 | 60 | 55 | 49 | 90 |
| KIT(A829P) | 77 | 100 | 54 | 69 | 75 | 64 | 75 |
| KIT(D816H) | 65 | 87 | 43 | 56 | 80 | 48 | 74 |
| KIT(D816V) | 96 | 59 | 34 | 56 | 76 | 36 | 67 |
| KIT(L576P) | 100 | 87 | 8.7 | 5.1 | 8.5 | 4.8 | 39 |
| KIT(V559D) | 100 | 91 | 47 | 43 | 45 | 36 | 75 |
| KIT(V559D, T670I) | 92 | 91 | 7.4 | 3.9 | 6.3 | 3.2 | 27 |
| KIT(V559D, V654A) | 100 | 67 | 83 | 64 | 59 | 54 | 73 |
| KIT-autoinhibited | 81 | 100 | 75 | 94 | 82 | 83 | 72 |
| PDGFRA | 80 | 95 | 70 | 72 | 79 | 56 | 72 |
| PDGFRB | 98 | 71 | 60 | 66 | 80 | 38 | 90 |
| RET | 85 | 63 | 24 | 27 | 33 | 23 | 8 |
| RET(M918T) | 95 | 59 | 26 | 32 | 36 | 25 | 6.4 |
| RET(V804L) | 100 | 43 | 58 | 55 | 60 | 50 | 37 |
| RET(V804M) | 97 | 43 | 69 | 81 | 77 | 64 | 57 |

TABLE 4

| | Ex. 138 | Ex. 139 | Ex. 140 | Ex. 141 | Ex. 142 | Ex. 143 | Ex. 144 |
|---|---|---|---|---|---|---|---|
| | (1 μM, | (1 μM, | (1 μM, | (1 μM, | (1 μM, | (1 μM, | (1 μM, |
| Target | % Ctrl) | % Ctrl) | % Ctrl) | % Ctrl) | % Ctrl) | % Ctrl) | % Ctrl) |
| ABL1(E255K)- | 61 | 4.7 | 0.5 | 0 | 3.2 | 6.4 | 0.1 |
| phosphorylated | | | | | | | |
| ABL1(F317I)- | 86 | 72 | 0 | 1.1 | 11 | 46 | 0.1 |
| nonphosphorylated | | | | | | | |
| ABL1(F317I)- | 100 | 60 | 11 | 4.1 | 49 | 26 | 1.5 |
| phosphorylated | | | | | | | |
| ABL1(F317L)- | 69 | 48 | 0 | 0 | 0 | 17 | 0 |
| nonphosphorylated | | | | | | | |
| ABL1(F317L)- | 75 | 39 | 8.2 | 2.3 | 30 | 17 | 0.8 |
| phosphorylated | | | | | | | |
| ABL1(H396P)- | 69 | 2 | 0 | 0 | 0.8 | 1.6 | 0 |
| nonphosphorylated | | | | | | | |
| ABL1(H396P)- | 82 | 9.5 | 1.4 | 0 | 7.2 | 11 | 0 |
| phosphorylated | | | | | | | |
| ABL1(M351T)- | 71 | 7.3 | 2.5 | 0.2 | 5.5 | 13 | 0.5 |
| phosphorylated | | | | | | | |
| ABL1(Q252H)- | 100 | 7.8 | 0 | 0.1 | 1.6 | 4.1 | 0 |
| nonphosphorylated | | | | | | | |
| ABL1(Q252H)- | 98 | 5.1 | 0.7 | 0.1 | 6.4 | 5.2 | 0.2 |
| phosphorylated | | | | | | | |
| ABL1(T315I)- | 87 | 0.1 | 0 | 0 | 0 | 42 | 0 |
| nonphosphorylated | | | | | | | |
| ABL1(T315I)- | 76 | 3 | 7.8 | 0.5 | 3.5 | 27 | 0.6 |
| phosphorylated | | | | | | | |
| ABL1(Y253F)- | 91 | 11 | 1 | 0 | 4.4 | 9.1 | 0.1 |
| phosphorylated | | | | | | | |
| ABL1- | 79 | 17 | 0 | 0.1 | 0.6 | 6 | 0 |
| nonphosphorylated | | | | | | | |
| ABL1-phosphorylated | 100 | 9.8 | 0.8 | 0.1 | 9.4 | 11 | 0 |
| ABL2 | 98 | 70 | 13 | 14 | 49 | 40 | 2.6 |
| EGFR | 76 | 94 | 86 | 26 | 81 | 100 | 92 |
| EGFR(E746-A750del) | 78 | 72 | 54 | 7.1 | 58 | 65 | 86 |
| EGFR(G719C) | 100 | 82 | 94 | 92 | 100 | 100 | 100 |
| EGFR(G719S) | 95 | 84 | 89 | 70 | 100 | 94 | 100 |
| EGFR(L747-E749del, | 100 | 71 | 86 | 3 | 96 | 90 | 71 |
| A750P) | | | | | | | |
| EGFR(L747-S752del, | 61 | 77 | 64 | 20 | 70 | 89 | 96 |
| P753S) | | | | | | | |
| EGFR(L747- | 96 | 81 | 82 | 32 | 100 | 96 | 99 |
| T751del, Sins) | | | | | | | |
| EGFR(L858R) | 78 | 80 | 79 | 24 | 77 | 86 | 92 |
| EGFR(L858R, T790M) | 82 | 17 | 96 | 0.8 | 6.3 | 75 | 50 |
| EGFR(L861Q) | 100 | 88 | 98 | 58 | 100 | 100 | 100 |
| EGFR(S752-I759del) | 92 | 74 | 93 | 34 | 100 | 86 | 78 |
| EGFR(T790M) | 94 | 36 | 95 | 3.8 | 17 | 100 | 82 |
| FLT1 | 96 | 65 | 56 | 6.7 | 63 | 11 | 4.5 |
| FLT3 | 77 | 33 | 10 | 0.6 | 86 | 15 | 0.4 |
| FLT3(D835H) | 73 | 14 | 2.7 | 0 | 16 | 0.8 | 0.3 |
| FLT3(D835V) | 18 | 0.9 | 0.7 | 0 | 0 | 0.3 | 0.2 |
| FLT3(D835Y) | 45 | 2.5 | 19 | 4.7 | 23 | 3.1 | 2.5 |
| FLT3(ITD) | 73 | 8.1 | 13 | 0 | 15 | 2.5 | 0.3 |
| FLT3(ITD, D835V) | 24 | 1.2 | 1.1 | 0 | 0.1 | 0.6 | 0.3 |
| FLT3(ITD, F691L) | 49 | 0.9 | 1.4 | 4.9 | 5.2 | 1 | 0 |
| FLT3(K663Q) | 95 | 44 | 5.7 | 0.6 | 49 | 11 | 13 |
| FLT3(N841I) | 68 | 12 | 0 | 0 | 0.6 | 5.5 | 4.1 |
| FLT3(R834Q) | 99 | 39 | 18 | 1.9 | 47 | 33 | 2 |
| FLT3-autoinhibited | 76 | 67 | 73 | 2.4 | 55 | 62 | 14 |
| FLT4 | 97 | 23 | 17 | 0 | 8.7 | 39 | 0.1 |
| KIT | 90 | 94 | 72 | 68 | 88 | 9.9 | 0.3 |
| KIT(A829P) | 100 | 74 | 74 | 40 | 66 | 51 | 11 |
| KIT(D816H) | 82 | 67 | 57 | 13 | 27 | 59 | 2.2 |
| KIT(D816V) | 100 | 63 | 19 | 11 | 33 | 34 | 0.8 |
| KIT(L576P) | 69 | 84 | 31 | 33 | 61 | 0 | 0 |
| KIT(V559D) | 97 | 91 | 69 | 59 | 82 | 5.4 | 0 |
| KIT(V559D, T670I) | 73 | 69 | 53 | 19 | 82 | 1.6 | 0.3 |
| KIT(V559D, V654A) | 62 | 75 | 60 | 63 | 70 | 33 | 8.1 |
| KIT-autoinhibited | 96 | 81 | 96 | 97 | 83 | 40 | 6.3 |
| PDGFRA | 75 | 74 | 30 | 11 | 43 | 41 | 3.6 |
| PDGFRB | 95 | 73 | 6 | 1.6 | 53 | 32 | 0.3 |
| RET | 63 | 42 | 0.6 | 0.1 | 34 | 0.6 | 0.1 |
| RET(M918T) | 72 | 41 | 0.3 | 0.1 | 48 | 0.7 | 0 |
| RET(V804L) | 83 | 15 | 1.6 | 0.1 | 14 | 3.5 | 0.2 |
| RET(V804M) | 81 | 23 | 6.5 | 0.7 | 13 | 31 | 3.1 |

TABLE 5

| | Ex. 145 | Ex. 146 | Ex. 147 | Ex. 148 | Ex. 149 | Ex. 150 | Ex. 151 |
|---|---|---|---|---|---|---|---|
| | (1 μM, | (1 μM, | (1 μM, | (1 μM, | (1 μM, | (1 μM, | (1 μM, |
| Target | % Ctrl) | % Ctrl) | % Ctrl) | % Ctrl) | % Ctrl) | % Ctrl) | % Ctrl) |
| ABL1(E255K)- | 0.1 | 57 | 0.3 | 86 | 74 | 95 | 36 |
| phosphorylated | | | | | | | |
| ABL1(F317I)- | 0 | 100 | 61 | 92 | 92 | 95 | 100 |
| nonphosphorylated | | | | | | | |
| ABL1(F317I)- | 3.2 | 100 | 18 | 89 | 90 | 88 | 82 |
| phosphorylated | | | | | | | |
| ABL1(F317L)- | 0 | 89 | 38 | 57 | 51 | 87 | 89 |
| nonphosphorylated | | | | | | | |
| ABL1(F317L)- | 0.7 | 89 | 8.2 | 92 | 93 | 82 | 80 |
| phosphorylated | | | | | | | |
| ABL1(H396P)- | 0 | 50 | 0.1 | 54 | 38 | 100 | 38 |
| nonphosphorylated | | | | | | | |
| ABL1(H396P)- | 0 | 80 | 0.4 | 92 | 90 | 100 | 0 |
| phosphorylated | | | | | | | |
| ABL1(M351T)- | 0.3 | 67 | 0.5 | 87 | 84 | 94 | 60 |
| phosphorylated | | | | | | | |
| ABL1(Q252H)- | 0 | 74 | 1.1 | 58 | 46 | 93 | 75 |
| nonphosphorylated | | | | | | | |
| ABL1(Q252H)- | 0.1 | 93 | 0.5 | 100 | 95 | 100 | 56 |
| phosphorylated | | | | | | | |
| ABL1(T315I)- | 0 | 64 | 0 | 72 | 75 | 93 | 30 |
| nonphosphorylated | | | | | | | |
| ABL1(T315I)- | 0.8 | 34 | 0.5 | 80 | 86 | 83 | 9.3 |
| phosphorylated | | | | | | | |
| ABL1(Y253F)- | 0.1 | 89 | 0.7 | 93 | 79 | 100 | 76 |
| phosphorylated | | | | | | | |
| ABL1- | 0 | 64 | 3 | 43 | 34 | 82 | 70 |
| nonphosphorylated | | | | | | | |
| ABL1-phosphorylated | 0.1 | 100 | 0.6 | 79 | 75 | 82 | 54 |
| ABL2 | 1.5 | 83 | 28 | 100 | 98 | 95 | 99 |
| EGFR | 53 | 51 | 42 | 48 | 79 | 61 | 56 |
| EGFR(E746-A750del) | 24 | 73 | 12 | 56 | 86 | 98 | 58 |
| EGFR(G719C) | 100 | 89 | 87 | 80 | 100 | 92 | 78 |
| EGFR(G719S) | 83 | 84 | 81 | 87 | 100 | 8 | 71 |
| EGFR(L747-E749del, | 19 | 38 | 4.2 | 54 | 83 | 94 | 73 |
| A750P) | | | | | | | |
| EGFR(L747-S752del, | 48 | 60 | 30 | 68 | 100 | 100 | 100 |
| P753S) | | | | | | | |
| EGFR(L747- | 88 | 77 | 54 | 38 | 93 | 94 | 37 |
| T751del, Sins) | | | | | | | |
| EGFR(L858R) | 94 | 88 | 59 | 87 | 100 | 77 | 85 |
| EGFR(L858R, T790M) | 64 | 43 | 5 | 70 | 83 | 83 | 39 |
| EGFR(L861Q) | 95 | 89 | 8 | 19 | 85 | 69 | 54 |
| EGFR(S752-I759del) | 82 | 83 | 68 | 81 | 100 | 100 | 100 |
| EGFR(T790M) | 89 | 57 | 19 | 64 | 100 | 86 | 62 |
| FLT1 | 11 | 100 | 30 | 100 | 94 | 100 | 93 |
| FLT3 | 4.4 | 24 | 8.6 | 97 | 86 | 71 | 64 |
| FLT3(D835H) | 1 | 5.7 | 0.5 | 80 | 64 | 62 | 23 |
| FLT3(D835V) | 0.5 | 0.2 | 0 | 15 | 16 | 13 | 1.2 |
| FLT3(D835Y) | 8.9 | 22 | 11 | 90 | 89 | 67 | 47 |
| FLT3(ITD) | 0.9 | 4.7 | 0.7 | 100 | 100 | 72 | 22 |
| FLT3(ITD, D835V) | 0 | 0.3 | 0 | 47 | 49 | 35 | 29 |
| FLT3(ITD, F691L) | 11 | 3.2 | 1.3 | 98 | 88 | 59 | 20 |
| FLT3(K663Q) | 2.5 | 14 | 5.3 | 89 | 88 | 71 | 53 |
| FLT3(N841I) | 0 | 0 | 0 | 88 | 81 | 19 | 9.5 |
| FLT3(R834Q) | 58 | 97 | 29 | 96 | 100 | 69 | 50 |
| FLT3-autoinhibited | 20 | 100 | 54 | 100 | 97 | 82 | 87 |
| FLT4 | 0.1 | 78 | 0.6 | 95 | 100 | 95 | 64 |
| KIT | 23 | 96 | 69 | 96 | 100 | 97 | 100 |
| KIT(A829P) | 17 | 92 | 37 | 91 | 78 | 96 | 85 |
| KIT(D816H) | 11 | 100 | 95 | 73 | 71 | 87 | 100 |
| KIT(D816V) | 4.1 | 80 | 23 | 100 | 96 | 89 | 79 |
| KIT(L576P) | 1.4 | 90 | 62 | 100 | 98 | 94 | 100 |
| KIT(V559D) | 11 | 75 | 60 | 100 | 100 | 88 | 100 |
| KIT(V559D, T670I) | 6.6 | 75 | 21 | 98 | 86 | 97 | 89 |
| KIT(V559D, V654A) | 42 | 97 | 78 | 100 | 100 | 100 | 100 |
| KIT-autoinhibited | 64 | 80 | 93 | 100 | 100 | 100 | 100 |
| PDGFRA | 27 | 100 | 71 | 97 | 96 | 92 | 89 |
| PDGFRB | 7.1 | 96 | 11 | 95 | 94 | 96 | 76 |
| RET | 0.1 | 45 | 6.8 | 84 | 49 | 93 | 68 |
| RET(M918T) | 0.1 | 27 | 3.5 | 99 | 64 | 90 | 63 |
| RET(V804L) | 0.1 | 3 | 1.1 | 100 | 91 | 77 | 48 |
| RET(V804M) | 1.9 | 12 | 7.8 | 100 | 100 | 68 | 74 |

TABLE 6

| | Ex. 152 | Ex. 153 | Ex. 154 | Ex. 55 | Ex. 156 | Ex. 158 | Ex. 159 |
|---|---|---|---|---|---|---|---|
| | (1 μM, | (1 μM, | (1 μM, | (1 μM, | (1 μM, | (1 μM, | (1 μM, |
| Target | % Ctrl) | % Ctrl) | % Ctrl) | % Ctrl) | % Ctrl) | % Ctrl) | % Ctrl) |
| ABL1(E255K)- | 0.1 | 8.4 | 0.1 | 2.6 | 7.9 | 61 | 100 |
| phosphorylated | | | | | | | |
| ABL1(F317I)- | 0.4 | 20 | 0 | 66 | 35 | 100 | 100 |
| nonphosphorylated | | | | | | | |
| ABL1(F317I)- | 0.7 | 37 | 4.7 | 41 | 56 | 88 | 91 |
| phosphorylated | | | | | | | |
| ABL1(F317L)- | 0 | 4.3 | 0 | 24 | 17 | 100 | 100 |
| nonphosphorylated | | | | | | | |
| ABL1(F317L)- | 0.8 | 18 | 1.2 | 10 | 32 | 65 | 72 |
| phosphorylated | | | | | | | |
| ABL1(H396P)- | 0 | 0.8 | 0.6 | 0.8 | 1.7 | 47 | 80 |
| nonphosphorylated | | | | | | | |
| ABL1(H396P)- | 0.1 | 16 | 0.1 | 3.6 | 9.5 | 7 | 88 |
| phosphorylated | | | | | | | |
| ABL1(M351T)- | 0.1 | 9.5 | 0.2 | 3.7 | 6.6 | 48 | 68 |
| phosphorylated | | | | | | | |
| ABL1(Q252H)- | 0 | 1.8 | 0.2 | 2.8 | 2.7 | 67 | 81 |
| nonphosphorylated | | | | | | | |
| ABL1(Q252H)- | 0.6 | 7.9 | 0.1 | 1.7 | 9.4 | 98 | 100 |
| phosphorylated | | | | | | | |
| ABL1(T315I)- | 0 | 17 | 0 | 1.6 | 1.5 | 88 | 100 |
| nonphosphorylated | | | | | | | |
| ABL1(T315I)- | 0.3 | 25 | 0.3 | 2.6 | 2.5 | 28 | 70 |
| phosphorylated | | | | | | | |
| ABL1(Y253F)- | 0.1 | 13 | 0.1 | 3.1 | 13 | 78 | 99 |
| phosphorylated | | | | | | | |
| ABL1- | 0.1 | 1.3 | 0.1 | 10 | 1.6 | 52 | 71 |
| nonphosphorylated | | | | | | | |
| ABL1-phosphorylated | 0 | 18 | 0.1 | 6 | 14 | 58 | 84 |
| ABL2 | 4.3 | 35 | 3.9 | 58 | 73 | 98 | 100 |
| EGFR | 52 | 61 | 56 | 59 | 59 | 78 | 95 |
| EGFR(E746-A750del) | 59 | 56 | 48 | 74 | 66 | 100 | 100 |
| EGFR(G719C) | 70 | 49 | 63 | 68 | 86 | 100 | 100 |
| EGFR(G719S) | 67 | 45 | 67 | 67 | 83 | 93 | 100 |
| EGFR(L747-E749del, | 33 | 85 | 77 | 82 | 90 | 100 | 100 |
| A750P) | | | | | | | |
| EGFR(L747-S752del, | 46 | 57 | 58 | 56 | 60 | 100 | 100 |
| P753S) | | | | | | | |
| EGFR(L747- | 58 | 38 | 48 | 66 | 80 | 100 | 100 |
| T751del, Sins) | | | | | | | |
| EGFR(L858R) | 51 | 86 | 89 | 88 | 90 | 100 | 100 |
| EGFR(L858R, T790M) | 1.9 | 79 | 21 | 35 | 35 | 87 | 97 |
| EGFR(L861Q) | 45 | 43 | 56 | 58 | 80 | 93 | 100 |
| EGFR(S752-I759del) | 69 | 100 | 88 | 84 | 98 | 100 | 84 |
| EGFR(T790M) | 8.4 | 83 | 48 | 70 | 41 | 71 | 85 |
| FLT1 | 9.6 | 3.5 | 12 | 62 | 77 | 93 | 96 |
| FLT3 | 0.1 | 1.6 | 32 | 11 | 7.5 | 100 | 100 |
| FLT3(D835H) | 0.3 | 0.3 | 0.1 | 2.7 | 7.5 | 63 | 92 |
| FLT3(D835V) | 11 | 0.3 | 0 | 1.1 | 1.3 | 49 | 100 |
| FLT3(D835Y) | 0.1 | 1.7 | 8.7 | 2 | 5 | 62 | 85 |
| FLT3(ITD) | 0 | 0.5 | 0.5 | 0.3 | 0.8 | 83 | 100 |
| FLT3(ITD, D835V) | 8.9 | 0.5 | 0.3 | 1.2 | 0.5 | 45 | 96 |
| FLT3(ITD, F691L) | 0.1 | 0.5 | 0.5 | 0.1 | 0.5 | 59 | 96 |
| FLT3(K663Q) | 0 | 1.4 | 4.3 | 1.2 | 3.1 | 44 | 76 |
| FLT3(N841I) | 0 | 0 | 0 | 0 | 0 | 72 | 100 |
| FLT3(R834Q) | 0.7 | 19 | 16 | 25 | 27 | 100 | 100 |
| FLT3-autoinhibited | 0.2 | 26 | 2.3 | 10 | 38 | 98 | 95 |
| FLT4 | 0 | 11 | 0.2 | 31 | 23 | 91 | 96 |
| KIT | 25 | 0.8 | 29 | 74 | 77 | 94 | 95 |
| KIT(A829P) | 15 | 43 | 21 | 39 | 36 | 100 | 92 |
| KIT(D816H) | 4.4 | 27 | 9.8 | 18 | 26 | 79 | 60 |
| KIT(D816V) | 0.7 | 6.8 | 1.6 | 1.3 | 3.8 | 100 | 100 |
| KIT(L576P) | 1.3 | 0.1 | 0.5 | 17 | 56 | 86 | 94 |
| KIT(V559D) | 10 | 0.3 | 13 | 55 | 76 | 95 | 96 |
| KIT(V559D, T670I) | 9.8 | 0.3 | 12 | 50 | 55 | 83 | 97 |
| KIT(V559D, V654A) | 3.2 | 13 | 4.9 | 22 | 65 | 100 | 100 |
| KIT-autoinhibited | 100 | 14 | 62 | 39 | 100 | 88 | 82 |
| PDGFRA | 4.6 | 9.1 | 29 | 50 | 39 | 89 | 92 |
| PDGFRB | 0 | 0.5 | 3.1 | 10 | 10 | 90 | 94 |
| RET | 0.8 | 2.4 | 1.2 | 4.2 | 75 | 81 | 96 |
| RET(M918T) | 0.3 | 1 | 0.2 | 0.6 | 66 | 92 | 100 |
| RET(V804L) | 0.1 | 6.1 | 0.1 | 0.3 | 15 | 66 | 98 |
| RET(V804M) | 0.1 | 41 | 0.4 | 0.3 | 19 | 75 | 92 |

TABLE 7

| | Ex. 160 | Ex. 161 | Ex. 162 | Ex. 163 | Ex. 164 | Ex. 165 | Ex. 166 |
|---|---|---|---|---|---|---|---|
| | (1 μM, | (1 μM, | (1 μM, | (1 μM, | (1 μM, | (1 μM, | (1 μM, |
| Target | % Ctrl) | % Ctrl) | % Ctrl) | % Ctrl) | % Ctrl) | % Ctrl) | % Ctrl) |
| ABL1(E255K)- | 0.3 | 86 | 99 | 6.8 | 0.1 | 5.3 | 1.8 |
| phosphorylated | | | | | | | |
| ABL1(F317I)- | 36 | 100 | 100 | 69 | 5.7 | 76 | 80 |
| nonphosphorylated | | | | | | | |
| ABL1(F317I)- | 4.8 | 91 | 88 | 38 | 6.7 | 69 | 31 |
| phosphorylated | | | | | | | |
| ABL1(F317L)- | 9.7 | 93 | 100 | 26 | 1.6 | 31 | 41 |
| nonphosphorylated | | | | | | | |
| ABL1(F317L)- | 2.5 | 94 | 96 | 24 | 1.3 | 23 | 8.7 |
| phosphorylated | | | | | | | |
| ABL1(H396P)- | 0 | 91 | 100 | 3.1 | 0 | 1.8 | 0.7 |
| nonphosphorylated | | | | | | | |
| ABL1(H396P)- | 0.1 | 100 | 100 | 7.2 | 0 | 7.8 | 4.3 |
| phosphorylated | | | | | | | |
| ABL1(M351T)- | 0.3 | 81 | 92 | 7.4 | 0.3 | 11 | 3.1 |
| phosphorylated | | | | | | | |
| ABL1(Q252H)- | 0.3 | 100 | 92 | 2.9 | 0.1 | 4.3 | 2.6 |
| nonphosphorylated | | | | | | | |
| ABL1(Q252H)- | 0 | 79 | 92 | 2.7 | 0 | 3.6 | 3.8 |
| phosphorylated | | | | | | | |
| ABL1(T315I)- | 0 | 90 | 100 | 63 | 0.6 | 59 | 1.4 |
| nonphosphorylated | | | | | | | |
| ABL1(T315I)- | 0.5 | 85 | 100 | 36 | 1.4 | 39 | 1.2 |
| phosphorylated | | | | | | | |
| ABL1(Y253F)- | 0.6 | 78 | 89 | 4.7 | 0.1 | 7 | 11 |
| phosphorylated | | | | | | | |
| ABL1- | 2.7 | 100 | 100 | 10 | 0.1 | 8.9 | 4.1 |
| nonphosphorylated | | | | | | | |
| ABL1-phosphorylated | 0.3 | 97 | 100 | 7.4 | 0.1 | 7 | 3.6 |
| ABL2 | 24 | 87 | 94 | 38 | 2.3 | 59 | 51 |
| EGFR | 100 | 73 | 76 | 66 | 95 | 100 | 70 |
| EGFR(E746-A750del) | 58 | 87 | 88 | 70 | 18 | 66 | 84 |
| EGFR(G719C) | 92 | 89 | 100 | 96 | 82 | 87 | 98 |
| EGFR(G719S) | 85 | 58 | 76 | 88 | 74 | 84 | 99 |
| EGFR(L747-E749del, | 67 | 100 | 100 | 100 | 19 | 33 | 62 |
| A750P) | | | | | | | |
| EGFR(L747-S752del, | 93 | 80 | 89 | 100 | 47 | 93 | 98 |
| P753S) | | | | | | | |
| EGFR(L747- | 86 | 93 | 100 | 90 | 45 | 80 | 90 |
| T751del, Sins) | | | | | | | |
| EGFR(L858R) | 100 | 100 | 100 | 100 | 45 | 70 | 85 |
| EGFR(L858R, T790M) | 14 | 96 | 95 | 86 | 80 | 100 | 8.1 |
| EGFR(L861Q) | 91 | 100 | 100 | 100 | 63 | 82 | 99 |
| EGFR(S752-I759del) | 85 | 81 | 98 | 100 | 55 | 89 | 93 |
| EGFR(T790M) | 44 | 100 | 100 | 92 | 97 | 100 | 26 |
| FLT1 | 56 | 82 | 100 | 26 | 13 | 49 | 79 |
| FLT3 | 2.4 | 100 | 99 | 19 | 2.5 | 38 | 4.2 |
| FLT3(D835H) | 2.8 | 48 | 76 | 5.3 | 0.3 | 1.2 | 0.7 |
| FLT3(D835V) | 8.2 | 23 | 83 | 0.1 | 1.1 | 1.5 | 1.8 |
| FLT3(D835Y) | 0.1 | 36 | 71 | 25 | 0.5 | 5.3 | 1.9 |
| FLT3(ITD) | 0.2 | 85 | 91 | 13 | 0.9 | 11 | 0.3 |
| FLT3(ITD, D835V) | 0.6 | 83 | 95 | 1.2 | 0.3 | 3.2 | 0.6 |
| FLT3(ITD, F691L) | 0 | 82 | 92 | 4.7 | 1 | 2.6 | 0 |
| FLT3(K663Q) | 0.5 | 54 | 74 | 19 | 1.4 | 16 | 1.7 |
| FLT3(N841I) | 0 | 39 | 77 | 2.5 | 0 | 0.8 | 0 |
| FLT3(R834Q) | 5.7 | 100 | 100 | 26 | 13 | 31 | 2.1 |
| FLT3-autoinhibited | 4.9 | 100 | 95 | 81 | 63 | 100 | 2 |
| FLT4 | 33 | 79 | 91 | 38 | 0.2 | 60 | 49 |
| KIT | 86 | 98 | 100 | 56 | 18 | 63 | 100 |
| KIT(A829P) | 35 | 95 | 97 | 52 | 33 | 80 | 28 |
| KIT(D816H) | 15 | 100 | 100 | 54 | 22 | 75 | 10 |
| KIT(D816V) | 7.5 | 100 | 97 | 47 | 3.3 | 51 | 2.8 |
| KIT(L576P) | 63 | 100 | 91 | 6.5 | 0.9 | 15 | 34 |
| KIT(V559D) | 89 | 92 | 81 | 43 | 6.9 | 55 | 54 |
| KIT(V559D, T670I) | 79 | 95 | 100 | 11 | 2.4 | 23 | 65 |
| KIT(V559D, V654A) | 74 | 87 | 98 | 66 | 27 | 68 | 43 |
| KIT-autoinhibited | 87 | 100 | 94 | 90 | 81 | 100 | 87 |
| PDGFRA | 52 | 100 | 99 | 60 | 30 | 84 | 50 |
| PDGFRB | 20 | 98 | 89 | 24 | 0.9 | 22 | 16 |
| RET | 15 | 97 | 96 | 3.5 | 0 | 0.9 | 7.1 |
| RET(M918T) | 8.2 | 92 | 99 | 2.2 | 0 | 1.3 | 2.9 |
| RET(V804L) | 2.1 | 72 | 91 | 8.7 | 1.2 | 20 | 0.3 |
| RET(V804M) | 0.5 | 83 | 86 | 37 | 2.1 | 71 | 0.1 |

TABLE 8

| | Ex. 167 | Ex. 168 | Ex. 169 | Ex. 170 | Ex. 171 | Ex. 172 |
|---|---|---|---|---|---|---|
| | (1 μM, | (1 μM, | (1 μM, | (1 μM, | (1 μM, | (1 μM, |
| Target | % Ctrl) | % Ctrl) | % Ctrl) | % Ctrl) | % Ctrl) | % Ctrl) |
| ABL1(E255K)- | 63 | 0.1 | 0.3 | 0.3 | 32 | 0.4 |
| phosphorylated | | | | | | |
| ABL1(F317I)- | 100 | 11 | 0.6 | 11 | 90 | 24 |
| nonphosphorylated | | | | | | |
| ABL1(F317I)- | 97 | 4.3 | 5 | 7.8 | 96 | 9.3 |
| phosphorylated | | | | | | |
| ABL1(F317L)- | 100 | 2.5 | 0.6 | 5.4 | 75 | 7.1 |
| nonphosphorylated | | | | | | |
| ABL1(F317L)- | 67 | 0.1 | 0.1 | 3.5 | 65 | 1.9 |
| phosphorylated | | | | | | |
| ABL1(H396P)- | 69 | 0 | 0 | 0.1 | 32 | 0 |
| nonphosphorylated | | | | | | |
| ABL1(H396P)- | 86 | 0.1 | 0 | 0.3 | 39 | 0.1 |
| phosphorylated | | | | | | |
| ABL1(M351T)- | 77 | 0.1 | 0.1 | 1.1 | 48 | 0.4 |
| phosphorylated | | | | | | |
| ABL1(Q252H)- | 76 | 0.2 | 0 | 0.1 | 27 | 0.3 |
| nonphosphorylated | | | | | | |
| ABL1(Q252H)- | 78 | 0.3 | 0.3 | 0.3 | 22 | 0.1 |
| phosphorylated | | | | | | |
| ABL1(T315I)- | 100 | 0.2 | 0.3 | 7.3 | 98 | 0 |
| nonphosphorylated | | | | | | |
| ABL1(T315I)- | 63 | 0.3 | 0.1 | 1.6 | 61 | 1.7 |
| phosphorylated | | | | | | |
| ABL1(Y253F)- | 79 | 0 | 0.1 | 0.3 | 36 | 0.2 |
| phosphorylated | | | | | | |
| ABL1- | 66 | 0 | 0 | 0.8 | 67 | 0.6 |
| nonphosphorylated | | | | | | |
| ABL1-phosphorylated | 68 | 0 | 0 | 0.3 | 41 | 1.4 |
| ABL2 | 90 | 4.3 | 0.3 | 12 | 83 | 4.2 |
| EGFR | 79 | 66 | 42 | 82 | 99 | 36 |
| EGFR(E746-A750del) | 100 | 20 | 9.9 | 19 | 84 | 43 |
| EGFR(G719C) | 100 | 100 | 100 | 93 | 97 | 100 |
| EGFR(G719S) | 100 | 100 | 88 | 95 | 95 | 82 |
| EGFR(L747-E749del, | 87 | 14 | 44 | 46 | 71 | 62 |
| A750P) | | | | | | |
| EGFR(L747-S752del, | 100 | 64 | 56 | 74 | 90 | 52 |
| P753S) | | | | | | |
| EGFR(L747- | 96 | 64 | 30 | 91 | 79 | 43 |
| T751del, Sins) | | | | | | |
| EGFR(L858R) | 87 | 62 | 52 | 70 | 89 | 48 |
| EGFR(L858R, T790M) | 41 | 22 | 37 | 73 | 85 | 3.3 |
| EGFR(L861Q) | 100 | 87 | 77 | 78 | 98 | 75 |
| EGFR(S752-I759del) | 99 | 89 | 61 | 86 | 100 | 37 |
| EGFR(T790M) | 68 | 51 | 69 | 86 | 90 | 22 |
| FLT1 | 98 | 5.1 | 12 | 21 | 90 | 26 |
| FLT3 | 26 | 6.1 | 1.8 | 4.2 | 58 | 8.5 |
| FLT3(D835H) | 0 | 0 | 0.1 | 6.1 | 47 | 4.2 |
| FLT3(D835V) | 0.8 | 0 | 0 | 1.7 | 3 | 0.5 |
| FLT3(D835Y) | 0.6 | 3.5 | 1.6 | 58 | 45 | 0.1 |
| FLT3(ITD) | 1.5 | 0.6 | 0.9 | 1.5 | 48 | 1.1 |
| FLT3(ITD, D835V) | 0.5 | 0 | 0.4 | 1.4 | 4.7 | 0.3 |
| FLT3(ITD, F691L) | 0.8 | 0.3 | 0.1 | 2.4 | 21 | 0.3 |
| FLT3(K663Q) | 4.6 | 3.6 | 2.7 | 18 | 94 | 0.6 |
| FLT3(N841I) | 0 | 0.1 | 0 | 2.8 | 36 | 0 |
| FLT3(R834Q) | 9 | 7.5 | 5.9 | 15 | 84 | 16 |
| FLT3-autoinhibited | 52 | 24 | 12 | 50 | 78 | 90 |
| FLT4 | 86 | 0.8 | 0.1 | 15 | 98 | 1.7 |
| KIT | 100 | 0.4 | 7.5 | 7.9 | 94 | 68 |
| KIT(A829P) | 51 | 25 | 22 | 42 | 85 | 31 |
| KIT(D816H) | 29 | 6.4 | 8.3 | 29 | 67 | 6.4 |
| KIT(D816V) | 12 | 1.6 | 1.6 | 18 | 81 | 0.8 |
| KIT(L576P) | 73 | 0 | 0 | 0 | 93 | 16 |
| KIT(V559D) | 74 | 0.1 | 1.2 | 2.5 | 91 | 42 |
| KIT(V559D, T670I) | 100 | 1.1 | 1.6 | 2 | 100 | 48 |
| KIT(V559D, V654A) | 100 | 4.9 | 13 | 40 | 90 | 40 |
| KIT-autoinhibited | 91 | 5.6 | 25 | 28 | 84 | 100 |
| PDGFRA | 78 | 5 | 16 | 27 | 80 | 44 |
| PDGFRB | 59 | 3.4 | 0.9 | 6.2 | 79 | 1.9 |
| RET | 94 | 0 | 0 | 0.9 | 88 | 3.1 |
| RET(M918T) | 66 | 0 | 0 | 0.6 | 60 | 0.8 |
| RET(V804L) | 42 | 0.1 | 0.2 | 7.3 | 81 | 0.5 |
| RET(V804M) | 15 | 0.7 | 0.9 | 29 | 94 | 0.8 |

### Cellular Assay

The inhibition of cellular activity of wild-type and mutant EGFRs will be evaluated at ProQinase GmbH (www.proqinase.com) using ProQinase's cellular phosphorylation assays that have been designed to measure compound activity in a physiological environment on a physiological substrate. The cellular kinase assays include EGFR wild-type, EGFR L858R mutant, EGFR T790M mutant, EGFR G719S mutant, EGFR L861Q mutant, EGFR Δ752-759 mutant, EGFR L858R/T790M mutant, EGFR Δ746-750/T790M mutant, EGFR Δ746-750/C₇₉₇S mutant, EGFR T790M/C₇₉₇S/L858R mutant, EGFR Δ746-750/T790M/C₇₉₇S mutant, and EGFR Δ747-749/A750P mutant. The detailed experimental protocols are available at ProQinase GmbH website.

1. A compound of the formula I, or a pharmaceutically acceptable salt thereof, 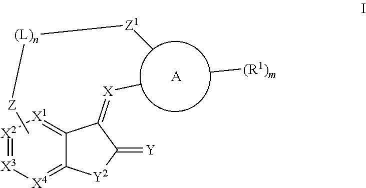
wherein
A is a 5- to 10-membered heteroarylene or C₆-C₁₀ arylene;
each L is independently —C(R³)(R⁴)—, —C(O)—, —O—, —N(R⁵)—, —S—, —S(O)— or —S(O)₂—, provided that (L)ₙ does not comprise a —O—O—, a —O—S—, or a —O—N(R⁵)— bond;
X is N or C(R⁶);
X¹ is N or C(R⁷);
X² is N or C(R⁸);
X³ is N or C(R⁹);
X⁴ is N or C(R¹⁰);
Y and Y¹ are each independently O or S;
Y² is —O—, —N(R¹¹)—, or —S—;
Z is a 3- to 7-membered heterocycloalkylene, C₃-C₆ cycloalkylene, C₆-C₁₀ arylene, 5- to 10-membered heteroarylene, —C(R¹²)(R¹³)—, —C(O)—, —O—, —N(R¹⁴)—, —S—, —S(O)— or —S(O)₂—, wherein each hydrogen atom in 3- to 7-membered heterocycloalkylene, C₃-C₆ cycloalkylene, C₆-C₁₀ arylene, and 5- to 10-membered heteroarylene is independently optionally substituted by deuterium, halogen, C₁-C₆ alkyl, C₁-C₆ haloalkyl, C₂-C₆ alkenyl, C₂-C₆ alkynyl, C₃-C₆ cycloalkyl, 3- to 7-membered heterocycloalkyl, —OR^{c}, —OC(O)R^{e}, —OC(O)NR^{e}R, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
Z¹ is —NR²C(Y¹)—, —C(Y¹)NR²—, —O—, —N(R²)—, —S—, —S(O)— or —S(O)₂—,
each R¹ is independently deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each of R², R^{5′} R¹¹, or R¹⁴ is independently H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}′, —CN, or —NO₂;
each R³, R⁴, R¹² and R¹³ is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SRC, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)ORC, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, —NO₂, or two of R³, R⁴, R¹², and R¹³ taken together with the carbon or carbons to which they are attached form a C₃-C₆ cycloalkyl or a 4- to 6-membered heterocycloalkyl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, or 4- to 6-membered heterocycloalkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R⁶ is H, deuterium, halogen, C₁-C₆ alkyl, C₂-C₆ alkenyl, C₂-C₆ alkynyl, or —CN;
each of R⁷ and R⁸ is independently a bond to Z, H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂; wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; provided that one of R⁷ or R⁸ is a bond to Z;
each of R⁹ and R¹⁰ is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂; or R⁸ and R⁹ or R⁹ and R¹⁰ taken together with the carbons to which they are attached form a C₄-C₆ cycloalkyl, a 4- to 7-membered heterocycloalkyl, or a C₆-C₁₀ aryl, wherein each hydrogen atom in C₁-C₆ alkyl, C₂-C₆ alkenyl, C₂-C₆ alkynyl, C₃-C₆ cycloalkyl, C₄-C₆ cycloalkyl, 3- to 7-membered heterocycloalkyl, C₆-C₁₀ aryl, 5- to 10-membered heteroaryl, or 4- to 7-membered heterocycloalkyl is independently optionally substituted by deuterium, halogen, C₁-C₆ alkyl, C₁-C₆ haloalkyl, —OR^{c}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each R^{a}, R^{b}, R^{c}, R^{d}, R^{c}, and R^{f} is independently selected from the group consisting of H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkyl-C₆-C₁₀ aryl, and 5- to 10-membered heteroaryl;
m is 0, 1, 2, 3, or 4; and
n is 2, 3, 4, 5, 6, 7, or 8.

2. The compound of claim 1 having the formula IV 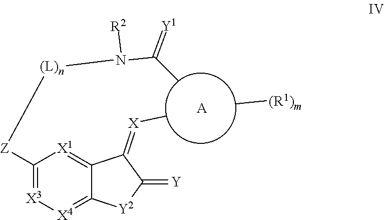
or a pharmaceutically acceptable salt thereof.

3. The compound of claim 1 having the formula VI 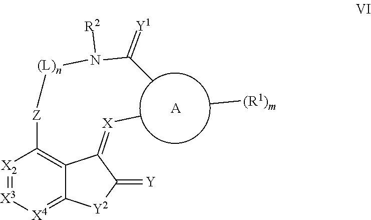
or a pharmaceutically acceptable salt thereof.

4. The compound of claim 1, or a pharmaceutically acceptable salt thereof, wherein A is phenylene, furanylene, thiophenylene, pyrrolylene, oxazolylene, isoxazolylene, thiazolylene, isothiazolylene, pyrazolylene, imidazolylene, oxadiazolylene, thiadiazolylene, triazolylene, pyridinylene, pyrazinylene, pyrimidinylene, pyridazinylene, or triazinylene.

5. (canceled)

6. The compound of claim 1, or a pharmaceutically acceptable salt thereof, wherein A is 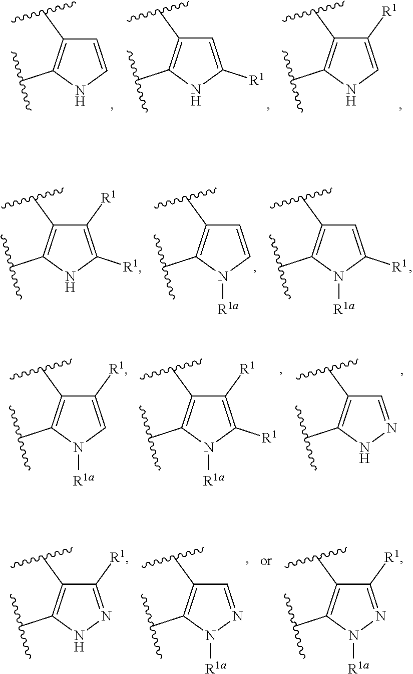
wherein R^{1a} is C₁-C₆ alkyl, —C(O)R^{a}, —C(O)OR^{a}, —C(O)NR^{a}R^{b}, or —P(O)₂OR^{a}, wherein each hydrogen atom in C₁-C₆ alkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

7. (canceled)

8. The compound of claim 1, or a pharmaceutically acceptable salt thereof, each R¹ is —CN or C₁-C₆ alkyl, wherein each hydrogen atom in C₁-C₆ alkyl is independently optionally substituted by deuterium, halogen, C₁-C₆ alkyl, C₁-C₆ haloalkyl, —OR^{c}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

9. (canceled)

10. (canceled)

11. The compound of claim 1, or a pharmaceutically acceptable salt thereof, wherein R² is H or C₁-C₆ alkyl, wherein each hydrogen atom in C₁-C₆ alkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{c}, —S(O)₂R^{c}, —S(O)NR^{e}R^{f}, —S(O)₂NR^{c}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

12. (canceled)

13. The compound of claim 1, or a pharmaceutically acceptable salt thereof, wherein Z is a 5- or 6-membered heteroarylene, wherein each hydrogen atom in 5- or 6-membered heteroarylene is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

14. (canceled)

15. (canceled)

16. The compound of claim 1, or a pharmaceutically acceptable salt thereof, wherein Z is C₆-C₁₀ arylene, wherein each hydrogen atom in C₆-C₁₀ arylene is independently optionally substituted by deuterium, halogen, C₁-C₆ alkyl, C₁-C₆ haloalkyl, —OR^{c}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

17.-20. (canceled)

21. The compound of claim 1, or a pharmaceutically acceptable salt thereof, wherein Z is —C(R¹²)(R¹³)—, —O—, —N(R¹⁴)—, —S—, —S(O)— or —S(O)₂—.

22.-39. (canceled)

40. The compound of claim 1, or a pharmaceutically acceptable salt thereof, wherein each L is independently selected from the group consisting of —C(O)—, —O—, —CH₂—, —C(H)(CH₃)—, —C(H)(OH)—, —C(H)(C(O)OR)—, —C(H)(C(O)NR^{c}R^{d})—, —NH—, and —NCH₃—.

41.-49. (canceled)

50. The compound of claim 1, or a pharmaceutically acceptable salt thereof, wherein X² is —C(R⁸), and R⁸ is a bond to Z.

51. (canceled)

52. (canceled)

53. The compound of claim 50, or a pharmaceutically acceptable salt thereof, wherein X¹ is C(R⁷), X³ is C(R⁹), and X⁴ is C(R¹⁰).

54.-57. (canceled)

58. The compound of claim 1, or a pharmaceutically acceptable salt thereof, wherein -(L)ₙ- is —(CH₂)₂—, —(CH₂)₃—, —(CH₂)₄—, —(CH₂)₅—, —(CH₂)₆—, —C(O)NH—(CH₂)₂O(CH₂)₂—, —C(O)N(CH₃)—(CH₂)₂O(CH₂)₂—, —NHC(O)CH₂O(CH₂)₂—, —N(CH₃)—C(O)CH₂O(CH₂)₂—, —CH₂O(CH₂)₂—, —(CH₂)₂O(CH₂)₂—, —(CH₂)₂S(CH₂)₂—, —O(CH₂)₂S(CH₂)₂—, —(CH₂)₂SO₂(CH₂)₂—, —O(CH₂)₂SO₂(CH₂)₂—, —(CH₂)₂SO(CH₂)₂—, —O(CH₂)₂SO(CH₂)₂—, —(CH₂)₂O(C(H)(C(O)N(H)(azetidin-3-yl))-CH₂—, —(CH₂)₂O(C(H)(C(O)N(H)(CH₃))—CH₂—, —(CH₂)₂O(C(H)(C(O)N(CH₃)₂)—CH₂—, —(CH₂)₂O(C(H)(C(O)N(H)(piperidin-4-yl))-CH₂—, —(CH₂)₂O(C(H)(C(O)N(H)(pyrrolidin-3-yl))-CH₂—, —(CH₂)₂O(C(H)(C(O)N(H)(4-methylpiperazin-1-yl))-CH₂—, —(CH₂)₂O(C(H)(C(O)OCH₃)—CH₂—, —(CH₂)₃O(CH₂)₂—, —(CH₂)₂O(CH₂)₃—, —CH₂CH(CH₃)—O(CH₂)₂—, —CH(CH₃)—CH₂O(CH₂)₂—, —O(CH₂)₂—, —O—(CH₂)₃—, —OCH₂O(CH₂)₂—, —O—CH₂CH(OH)CH₂—, —O—(CH₂)₂O(CH₂)₂—, —O—CH₂CH(CH₃)—O(CH₂)₂—, —O—CH(CH₃)—CH₂O(CH₂)₂—, —O—(CH₂)₂NH—(CH₂)₂—, —O—CH₂CH(CH₃)—NH—(CH₂)₂—, —O—CH(CH₃)—CH₂NH—(CH₂)₂—, —CH₂NH—(CH₂)₂—, —(CH₂)₂NH—(CH₂)₂—, —CH₂CH(CH₃)—NH—(CH₂)₂—, —CH(CH₃)—CH₂NH—(CH₂)₂—, —O—(CH₂)₂N(CH₃)—(CH₂)₂—, —O—CH₂CH(CH₃)—N(CH₃)—(CH₂)₂—, —O—CH(CH₃)—CH₂N(CH₃)—(CH₂)₂—, —CH₂N(CH₃)—(CH₂)₂—, —CH₂N(CH₂CH₃)—(CH₂)₂—, —CH₂N(CH(CH₃))—(CH₂)₂—, —(CH₂)₂N(CH₃)—(CH₂)₂—, —CH₂CH(CH₃)—N(CH₃)—(CH₂)₂—, or —O—CH(CH₃)—CH₂N(CH₃)—(CH₂)₂—.

59. The compound of claim 1, or a pharmaceutically acceptable salt thereof, selected from the group consisting of [3a(4)Z]-10,11-dihydro-2H,13H-16,1-(azenometheno)pyrazolo[4,3-m]dipyrrolo[3,2 f:3′,4′-i][11,4,11]oxadiazacyclotetradecine-3,8(5H,9H)-dione;
[3a(4)Z]-9,10,11,12-tetrahydro-14H-17,1-(azenometheno)pyrazolo[3,4-b]dipyrrolo[3,4-f:2′,3′-i][1,5,12]oxadiazacyclopentadecine-3,8(2H,5H)-dione;
[3a(4)Z]-9,10,11,12-tetrahydro-14H-1,17-(azenometheno)pyrazolo[3,4-b]dipyrrolo[3,4-f:2′,3′-i][1,5,12]oxadiazacyclopentadecine-3,8(2H,5H)-dione;
[3a(4)Z]-9,10,11,12-tetrahydro-14H-1,17-(diazanediylidene)pyrazolo[4,3-n]dipyrrolo[3,2-g:3′,4′-j][1,5]oxazacyclopentadecine-3,8(2H,5H)-dione;
[3a(4)Z]-9,10,11,12-tetrahydro-14H-1,17-(ethanediylidene)pyrazolo[3,4-b]dipyrrolo[3,4-f:2′,3′-i][1,5,12]oxadiazacyclopentadecine-3,8(2H,5H)-dione;
[3a(4)Z]-9,10,11,12-tetrahydro-14H-17,1-(azenometheno)pyrazolo[4,3-n]dipyrrolo[3,2-g:3′,4′-j][1,5]oxazacyclopentadecine-3,8(2H,5H)-dione;
[3a(4)Z]-9,10,11,12-tetrahydro-14H-1,17-(azenometheno)pyrazolo[4,3-n]dipyrrolo[3,2-g:3′,4′-j][1,5]oxazacyclopentadecine-3,8(2H,5H)-dione;
[3a(4)Z]-9,10,11,12-tetrahydro-14H-1,17-(ethanediylidene)pyrazolo[4,3-n]dipyrrolo[3,2-g:3′,4′-j][1,5]oxazacyclopentadecine-3,8(2H,5H)-dione;
[3a(4)Z,11S]-11-hydroxy-9,10,11,12-tetrahydro-14H-1,17-(ethanediylidene)pyrazolo[3,4-b]dipyrrolo[3,4-f:2′,3′-i][1,5,12]oxadiazacyclopentadecine-3,8(2H,5H)-dione;
[19a(20)Z]-2,5-dimethyl-6,7,9,10-tetrahydro-1H,12H-15,17-(ethanediylidene)pyrazolo[4,3-p]dipyrrolo[3,2-i:3′,4′-l][1,4,7,14]dioxadiazacycloheptadecine-4,19(5H,18H)-dione;
[3a(4)Z]-6-methyl-9,10,11,12-tetrahydro-14H-1,17-(ethanediylidene)pyrazolo[3,4-b]dipyrrolo[3,4-f:2′,3′-i][1,5,12]oxadiazacyclopentadecine-3,8(2H,5H)-dione;
[3a(4)Z]-3,8-dioxo-2,3,5,8,9,10,11,12-octahydro-14H-1,17-(ethanediylidene)pyrazolo[3,4-b]dipyrrolo[3,4-f:2′,3′-i][1,5,12]oxadiazacyclopentadecine-6-carbonitrile;
[3a(4)Z]-6-methyl-9,10,11,12-tetrahydro-14H-1,17-(ethanediylidene)imidazo[4,5-i]pyrazolo[3,4-b]pyrrolo[3,4-f][1,5,12]oxadiazacyclopentadecine-3,8(2H,5H)-dione;
[3a(4)Z]-6,15-dimethyl-9,10,11,12-tetrahydro-15H-1,17-(ethanediylidene)pyrazolo[3,4-b]dipyrrolo[3,4-f:2′,3′-i][1,5,12]oxadiazacyclopentadecine-3,8(2H,5H)-dione;
[3a(4)Z]-6-methyl-9,10,11,12-tetrahydro-1,17-(ethanediylidene)[1,2]oxazolo[3,4-b]dipyrrolo[3,4-f:2′,3′-i][1,5,12]oxadiazacyclopentadecine-3,8(2H,5H)-dione;
[3a(4)Z]-6,16-dimethyl-9,10,11,12-tetrahydro-1,17-(ethanediylidene)[1,2]oxazolo[3,4-b]dipyrrolo[3,4-f:2′,3′-i][1,5,12]oxadiazacyclopentadecine-3,8(2H,5H)-dione;
[3a(4)Z]-6-methyl-9,10,11,12-tetrahydro-1,17-(ethanediylidene)dipyrrolo[3,4-f:2′,3′-i][1,2]thiazolo[3,4-b][1,5,12]oxadiazacyclopentadecine-3,8(2H,5H)-dione;
[3a(4)Z]-6,9-dimethyl-9,10,11,12-tetrahydro-14H-1,17-(ethanediylidene)pyrazolo[3,4-b]dipyrrolo[3,4-f:2′,3′-i][1,5,12]oxadiazacyclopentadecine-3,8(2H,5H)-dione;
[3a(4)Z]-6,9-dimethyl-9,10,11,12-tetrahydro-1,17-(ethanediylidene)[1,2]oxazolo[3,4-b]dipyrrolo[3,4-f:2′,3′-i][1,5,12]oxadiazacyclopentadecine-3,8(2H,5H)-dione;
[3a(4)Z]-6,9,16-trimethyl-9,10,11,12-tetrahydro-14H-1,17-(ethanediylidene)pyrazolo[3,4-b]dipyrrolo[3,4-f:2′,3′-i][1,5,12]oxadiazacyclopentadecine-3,8(2H,5H)-dione;
[3a(4)Z]-6,9,16-trimethyl-9,10,11,12-tetrahydro-1,17-(ethanediylidene)[1,2]oxazolo[3,4-b]dipyrrolo[3,4-f:2′,3′-i][1,5,12]oxadiazacyclopentadecine-3,8(2H,5H)-dione;
[3a(4)Z]-6-methyl-9,10,11,12-tetrahydro-1,17-(ethanediylidene)pyrazolo[5,1-c]dipyrrolo[3,2-j:3′,4′-m][1,4,8]triazacyclotetradecine-3,8(2H,5H)-dione;
[3a(4)Z]-6-methyl-9,10,11,12-tetrahydro-17,1-(azenometheno)pyrazolo[1,5-e]dipyrrolo[3,4-i:2′,3′-l][1,5]diazacyclotetradecine-3,8(2H,5H)-dione;
[19a(20)Z]-2-methyl-6,7,9,10-tetrahydro-1H-15,17-(ethanediylidene)pyrazolo[1,5-d]dipyrrolo[3,4-h:2′,3′-k][1,4,7,14]oxatriazacyclohexadecine-4,19(5H,18H)-dione;
[19a(20)Z]-2-methyl-6,7,9,10-tetrahydro-1H-15,17-(azenometheno)pyrazolo[1,5-d]dipyrrolo[3,4-h:2′,3′-k][1,4,14]oxadiazacyclohexadecine-4,19(5H,18H)-dione;
[19a(20)Z]-2-methyl-6,7,9,10-tetrahydro-1H-15,17-(azenometheno)pyrazolo[1,5-d]dipyrrolo[3,4-h:2′,3′-k][1,4,7,14]oxatriazacyclohexadecine-4,19(5H,18H)-dione;
[19a(20)Z]-2-methyl-6,7,9,10-tetrahydro-1H-15,17-(ethanediylidene)pyrazolo[1,5-d]dipyrrolo[3,4-h:2′,3′-k][1,4,14]oxadiazacyclohexadecine-4,19(5H,18H)-dione;
[10R,19a(20)Z]-2,10-dimethyl-6,7,9,10-tetrahydro-1H-15,17-(ethanediylidene)pyrazolo[1,5-d]dipyrrolo[3,4-h:2′,3′-k][1,4,14]oxadiazacyclohexadecine-4,19(5H,18H)-dione;
[19a(20)Z]-2,5-dimethyl-6,7,9,10-tetrahydro-1H-15,17-(ethanediylidene)pyrazolo[1,5-d]dipyrrolo[3,4-h:2′,3′-k][1,4,14]oxadiazacyclohexadecine-4,19(5H,18H)-dione;
[19a(20)Z]-2,5-dimethyl-6,7,9,10-tetrahydro-1H-15,17-(azenometheno)pyrazolo[1,5-d]dipyrrolo[3,4-h:2′,3′-k][1,4,7,14]oxatriazacyclohexadecine-4,19(5H,18H)-dione;
[19a(20)Z]-2-methyl-5,6,7,8,9,10-hexahydro-15,17-(ethanediylidene)pyrazolo[1,5-g]dipyrrolo[3,4-k:2′,3′-n][1,4,7]triazacyclohexadecine-4,19(1H,18H)-dione;
[19a(20)Z]-2,5-dimethyl-5,6,7,8,9,10-hexahydro-15,17-(ethanediylidene)pyrazolo[1,5-g]dipyrrolo[3,4-k:2′,3′-n][1,4,7]triazacyclohexadecine-4,19(1H,18H)-dione;
[3a(4)Z]-6-methyl-10,11,13,14-tetrahydro-2H-1,17-(ethanediylidene)pyrazolo[4,3-m]dipyrrolo[3,2-f:3′,4′-i][1,4,11]oxadiazacyclopentadecine-3,8(5H,9H)-dione;
[3a(4)Z]-6-methyl-10,11,13,14-tetrahydro-2H-17,1-(azenometheno)pyrazolo[4,3-m]dipyrrolo[3,2-f:3′,4′-i][1,4]oxazacyclopentadecine-3,8(5H,9H)-dione;
[3a(4)Z]-6-methyl-10,11,13,14-tetrahydro-2H-1,17-(ethanediylidene)pyrazolo[4,3-m]dipyrrolo[3,2-f:3′,4′-i][1,4]oxazacyclopentadecine-3,8(5H,9H)-dione;
[3a(4)Z]-6,9-dimethyl-10,11,13,14-tetrahydro-2H-1,17-(ethanediylidene)pyrazolo[4,3-m]dipyrrolo[3,2-f:3′,4′-i][1,4]oxazacyclopentadecine-3,8(5H,9H)-dione;
[3a(4)Z]-6,9,16-trimethyl-10,11,13,14-tetrahydro-2H-1,17-(ethanediylidene)pyrazolo[4,3-m]dipyrrolo[3,2-f:3′,4′-i][1,4]oxazacyclopentadecine-3,8(5H,9H)-dione;
[3a(4)Z]-6-methyl-9,10,11,12,13,14-hexahydro-17,1-(azenometheno)pyrazolo[3,4-f]dipyrrolo[3,4-j:2′,3′-m][1,4,9]triazacyclopentadecine-3,8(2H,5H)-dione;
[3a(4)Z]-6-methyl-9,10,11,12,13,14-hexahydro-1,17-(ethanediylidene)pyrazolo[3,4-f]dipyrrolo[3,4-j:2′,3′-m][1,4,9]triazacyclopentadecine-3,8(2H,5H)-dione;
[3a(4)Z]-6-methyl-9,10,11,12,13,14-hexahydro-17,1-(azenometheno)pyrazolo[4,3-m]dipyrrolo[3,2-f:3′,4′-i][1,4]diazacyclopentadecine-3,8(2H,5H)-dione;
[3a(4)Z]-6,9-dimethyl-9,10,11,12,13,14-hexahydro-17,1-(azenometheno)pyrazolo[3,4-f]dipyrrolo[3,4-j:2′,3′-m][1,4,9]triazacyclopentadecine-3,8(2H,5H)-dione;
[3a(4)Z]-6,9-dimethyl-9,10,11,12,13,14-hexahydro-1,17-(ethanediylidene)pyrazolo[3,4-f]dipyrrolo[3,4-j:2′,3′-m][1,4,9]triazacyclopentadecine-3,8(2H,5H)-dione;
[3a(4)Z]-6,9-dimethyl-9,10,11,12,13,14-hexahydro-17,1-(azenometheno)pyrazolo[4,3-m]dipyrrolo[3,2-f:3′,4′-i][1,4]diazacyclopentadecine-3,8(2H,5H)-dione;
[3a(4)Z]-6,9-dimethyl-9,10,11,12,13,14-hexahydro-1,17-(ethanediylidene)pyrazolo[4,3-m]dipyrrolo[3,2-f:3′,4′-i][1,4]diazacyclopentadecine-3,8(2H,5H)-dione;
[3a(4)Z]-20-fluoro-6,9-dimethyl-9,10,11,12,13,14-hexahydro-1,17-(ethanediylidene)pyrazolo[4,3-m]dipyrrolo[3,2-f:3′,4′-i][1,4]diazacyclopentadecine-3,8(2H,5H)-dione;
[3a(4)Z]-19-fluoro-6,9-dimethyl-9,10,11,12,13,14-hexahydro-1,17-(ethanediylidene)pyrazolo[4,3-m]dipyrrolo[3,2-f:3′,4′-i][1,4]diazacyclopentadecine-3,8(2H,5H)-dione;
[3a(4)Z]-6,9,20-trimethyl-9,10,11,12,13,14-hexahydro-17,1-(azenometheno)pyrazolo[4,3-m]dipyrrolo[3,2-f:3′,4′-i][1,4]diazacyclopentadecine-3,8(2H,5H)-dione;
[3a(4)Z]-9,20-dimethyl-9,10,11,12,13,14-hexahydro-1,17-(ethanediylidene)pyrazolo[3,4-f]dipyrrolo[3,4-j:2′,3′-m][1,4,9]triazacyclopentadecine-3,8(2H,5H)-dione;
[3a(4)Z]-6,16-dimethyl-9,10,11,12,13,14-hexahydro-17,1-(azenometheno)pyrazolo[4,3-m]dipyrrolo[3,2-f:3′,4′-i][1,4]diazacyclopentadecine-3,8(2H,5H)-dione;
[3a(4)Z]-6,9,16-trimethyl-9,10,11,12,13,14-hexahydro-17,1-(azenometheno)pyrazolo[4,3-m]dipyrrolo[3,2-f:3′,4′-i][1,4]diazacyclopentadecine-3,8(2H,5H)-dione;
[3a(4)Z]-16-cyclopropyl-6,9-dimethyl-9,10,11,12,13,14-hexahydro-17,1-(azenometheno)pyrazolo[4,3-m]dipyrrolo[3,2-f:3′,4′-i][1,4]diazacyclopentadecine-3,8(2H,5H)-dione;
[3a(4)Z]-16-cyclopropyl-6,9-dimethyl-9,10,11,12,13,14-hexahydro-1,17-(ethanediylidene)pyrazolo[3,4-f]dipyrrolo[3,4-j:2′,3′-m][1,4,9]triazacyclopentadecine-3,8(2H,5H)-dione;
[3a(4)Z]-6,9,16-trimethyl-10,11,12,13-tetrahydro-2H-17,1-(azenometheno)[1,2]oxazolo[4,5-m]dipyrrolo[3,2-f:3′,4′-i][1,4]diazacyclopentadecine-3,8(5H,9H)-dione;
[3a(4)Z]-6,9,16-trimethyl-10,11,12,13-tetrahydro-2H-17,1-(azenometheno) [1,2]oxazolo[4,3-m]dipyrrolo[3,2-f:3′,4′-i][1,4]diazacyclopentadecine-3,8(5H,9H)-dione;
[3a(4)Z]-6,14-dimethyl-10,11,13,14-tetrahydro-2H-17,1-(azenometheno)pyrazolo[4,3-m]dipyrrolo[3,2-f:3′,4′-i][1,4]oxazacyclopentadecine-3,8(5H,9H)-dione;
[3a(4)Z]-6,9,14-trimethyl-10,11,13,14-tetrahydro-2H-1,17-(ethanediylidene)pyrazolo[4,3-m]dipyrrolo[3,2-f:3′,4′-i][1,4]oxazacyclopentadecine-3,8(5H,9H)-dione;
[3a(4)Z]-6,9,14-trimethyl-9,10,11,12,13,14-hexahydro-1,17-(ethanediylidene)pyrazolo[4,3-m]dipyrrolo[3,2-f:3′,4′-i][1,4]diazacyclopentadecine-3,8(2H,5H)-dione;
[3a(4)Z]-6,9,12,14-tetramethyl-9,10,11,12,13,14-hexahydro-1,17-(ethanediylidene)pyrazolo[4,3-m]dipyrrolo[3,2-f:3′,4′-i][1,4]diazacyclopentadecine-3,8(2H,5H)-dione;
[3a(4)Z]-6,9,16-trimethyl-9,10,11,12,13,14-hexahydro-1,17-(ethanediylidene)pyrazolo[4,3-m]dipyrrolo[3,2-f:3′,4′-i][1,4]diazacyclopentadecine-3,8(2H,5H)-dione;
[3a(4)Z]-6,9,16-trimethyl-10,11-dihydro-2H,13H-1,17-(ethanediylidene)[1,2]oxazolo[4,3-m]dipyrrolo[3,2-f:3′,4′-i][1,4]oxazacyclopentadecine-3,8(5H,9H)-dione;
[3a(4)Z]-6,9,16-trimethyl-10,11,12,13-tetrahydro-2H-1,17-(ethanediylidene)[1,2]oxazolo[4,3-m]dipyrrolo[3,2-f:3′,4′-i][1,4]diazacyclopentadecine-3,8(5H,9H)-dione;
[3a(4)Z]-6,9,12,14,16-pentamethyl-9,10,11,12,13,14-hexahydro-1,17-(ethanediylidene)pyrazolo[4,3-m]dipyrrolo[3,2-f:3′,4′-i][1,4]diazacyclopentadecine-3,8(2H,5H)-dione;
[3a(4)Z]-6,9,14,16-tetramethyl-9,10,11,12-tetrahydro-14H-1,17-(ethanediylidene)pyrazolo[4,3-n]dipyrrolo[3,2-g:3′,4′-j][1,5]oxazacyclopentadecine-3,8(2H,5H)-dione;
[3a(4)Z]-6,9,14,16-tetramethyl-10,11,13,14-tetrahydro-2H-1,17-(ethanediylidene)pyrazolo[4,3-m]dipyrrolo[3,2-f:3′,4′-i][1,4]oxazacyclopentadecine-3,8(5H,9H)-dione;
[3a(4)Z]-9,14,16-trimethyl-9,10,11,12-tetrahydro-14H-1,17-(ethanediylidene)pyrazolo[4,3-n]dipyrrolo[3,2-g:3′,4′-j][1,5]oxazacyclopentadecine-3,8(2H,5H)-dione;
[3a(4)Z]-9,14,16-trimethyl-10,11,13,14-tetrahydro-2H-1,17-(ethanediylidene)pyrazolo[4,3-m]dipyrrolo[3,2-f:3′,4′-i][1,4]oxazacyclopentadecine-3,8(5H,9H)-dione;
[3a(4)Z]-12-ethyl-6,9,14-trimethyl-9,10,11,12,13,14-hexahydro-1,17-(ethanediylidene)pyrazolo[4,3-m]dipyrrolo[3,2-f:3′,4′-i][1,4]diazacyclopentadecine-3,8(2H,5H)-dione;
[3a(4)Z]-6,9,14-trimethyl-12-(propan-2-yl)-9,10,11,12,13,14-hexahydro-1,17-(ethanediylidene)pyrazolo[4,3-m]dipyrrolo[3,2-f:3′,4′-i][1,4]diazacyclopentadecine-3,8(2H,5H)-dione;
[3a(4)Z]-16-cyclopropyl-6,9-dimethyl-10,11-dihydro-2H,13H-1,17-(ethanediylidene)[1,2]oxazolo[4,3-m]dipyrrolo[3,2-f:3′,4′-i][1,4]oxazacyclopentadecine-3,8(5H,9H)-dione;
[3a(4)Z]-9,14-dimethyl-9,10,11,12,13,14-hexahydro-1,17-(ethanediylidene)pyrazolo[4,3-m]dipyrrolo[3,2-f:3′,4′-i][1,4]diazacyclopentadecine-3,8(2H,5H)-dione;
[3a(4)Z]-6,9-dimethyl-16-(propan-2-yl)-10,11-dihydro-2H,13H-1,17-(ethanediylidene)[1,2]oxazolo[4,3-m]dipyrrolo[3,2-f:3′,4′-i][1,4]oxazacyclopentadecine-3,8(5H,9H)-dione;
[3a(4)Z]-9-methyl-16-(propan-2-yl)-10,11-dihydro-2H,13H-1,17-(ethanediylidene)[1,2]oxazolo[4,3-m]dipyrrolo[3,2-f:3′,4′-i][1,4]oxazacyclopentadecine-3,8(5H,9H)-dione;
[3a(4)Z]-6,9,14-trimethyl-9,10,11,12-tetrahydro-14H-1,17-(ethanediylidene)pyrazolo[4,3-n]dipyrrolo[3,2-g:3′,4′-j][1,5]oxazacyclopentadecine-3,8(2H,5H)-dione;
[3a(4)Z]-9,14-dimethyl-9,10,11,12-tetrahydro-14H-1,17-(ethanediylidene)pyrazolo[4,3-n]dipyrrolo[3,2-g:3′,4′-j][1,5]oxazacyclopentadecine-3,8(2H,5H)-dione;
[3a(4)Z]-6,9,12,14-tetramethyl-9,10,11,12,13,14-hexahydro-17,1-(azenometheno)pyrazolo[4,3-m]dipyrrolo[3,2-f:3′,4′-i][1,4]diazacyclopentadecine-3,8(2H,5H)-dione;
[3a(4)Z]-9,12,14-trimethyl-9,10,11,12,13,14-hexahydro-17,1-(azenometheno)pyrazolo[4,3-m]dipyrrolo[3,2-f:3′,4′-i][1,4]diazacyclopentadecine-3,8(2H,5H)-dione;
[3a(4)Z]-6,9,12,14-tetramethyl-9,10,11,12,13,14-hexahydro-1,17-(ethanediylidene)pyrazolo[3,4-f]dipyrrolo[3,4-j:2′,3′-m][1,4,9]triazacyclopentadecine-3,8(2H,5H)-dione; and
[3a(4)Z]-9,12,14-trimethyl-9,10,11,12,13,14-hexahydro-1,17-(ethanediylidene)pyrazolo[3,4-f]dipyrrolo[3,4-j:2′,3′-m][1,4,9]triazacyclopentadecine-3,8(2H,5H)-dione.

60. The compound of claim 1, or a pharmaceutically acceptable salt thereof, selected from the group consisting of [3a(4)Z]-6-methyl-9,10,11,12-tetrahydro-1,18-(ethanediylidene)dipyrrolo[3,2-g:3′,4′-j][1,5,12]benzoxadiazacyclopentadecine-3,8(2H,5H)-dione;
[3a(4)Z]-6-methyl-10,11-dihydro-2H-1,17-(ethanediylidene)dipyrrolo[3,2-f:3′,4′-i][1,4,11]benzoxadiazacyclotetradecine-3,8(5H,9H)-dione;
[3a(4)Z]-6-methyl-10,11-dihydro-2H-17,1-(azenometheno)dipyrrolo[3,2-f:3′,4′-i][1,4]benzoxazacyclotetradecine-3,8(5H,9H)-dione;
[3a(4)Z]-16-fluoro-6-methyl-10,11-dihydro-2H-1,17-(ethanediylidene)dipyrrolo[3,2-f:3′,4′-i][1,4,11]benzoxadiazacyclotetradecine-3,8(5H,9H)-dione;
[3a(4)Z]-15-fluoro-6-methyl-10,11-dihydro-2H-1,17-(ethanediylidene)dipyrrolo[3,2-f:3′,4′-i][1,4,11]benzoxadiazacyclotetradecine-3,8(5H,9H)-dione;
[3a(4)Z]-14-fluoro-6-methyl-10,11-dihydro-2H-1,17-(ethanediylidene)dipyrrolo[3,2-f:3′,4′-i][1,4,11]benzoxadiazacyclotetradecine-3,8(5H,9H)-dione;
[3a(4)Z]-13-fluoro-6-methyl-10,11-dihydro-2H-1,17-(ethanediylidene)dipyrrolo[3,2-f:3′,4′-i][1,4,11]benzoxadiazacyclotetradecine-3,8(5H,9H)-dione; and
[3a(4)Z]-6,9,12-trimethyl-10,11,12,13-tetrahydro-2H-1,18-(ethanediylidene)dipyrrolo[3,2-g:3′,4′-j][2,5]benzodiazacyclopentadecine-3,8(5H,9H)-dione.

61. The compound of claim 1, or a pharmaceutically acceptable salt thereof, selected from the group consisting of [3a(4)Z]-6-methyl-10,11-dihydro-2H-1,17-(ethanediylidene)pyrido[3,2-m]dipyrrolo[3,2-f:3′,4′-i][1,4,11]oxadiazacyclotetradecine-3,8(5H,9H)-dione;
[3a(4)Z]-6-methyl-10,11-dihydro-2H-1,17-(ethanediylidene)pyrimido[5,4-m]dipyrrolo[3,2-f:3′,4′-i][1,4,11]oxadiazacyclotetradecine-3,8(5H,9H)-dione;
[3a(4)Z]-6,16-dimethyl-10,11-dihydro-2H-1,17-(ethanediylidene)pyrido[3,4-m]dipyrrolo[3,2-f:3′,4′-i][1,4,11]oxadiazacyclotetradecine-3,8(5H,9H)-dione;
[3a(4)Z]-6-methyl-9,10,11,12-tetrahydro-14H-1,18-(ethanediylidene)pyrido[2,1-c]dipyrrolo[3,2-j:3′,4′-m][1,4,8]triazacyclotetradecine-3,8,14(2H,5H)-trione;
[3a(4)Z]-6-methyl-9,10,11,12-tetrahydro-14H-18,1-(azenometheno)pyrido[1,2-e]dipyrrolo[3,4-i:2′,3′-l][1,5]diazacyclotetradecine-3,8,14(2H,5H)-trione;
or a pharmaceutically acceptable salt thereof.

62. The compound of claim 1, or a pharmaceutically acceptable salt thereof, selected from the group consisting of [3a(4)Z,13aR]-6-methyl-10,11,12,13,13a,14,15,16-octahydro-2H-18,1-(azenometheno)tripyrrolo[1,2-a:3′,2′-i:3″,4″-l][1,4,7]triazacyclopentadecine-3,8(5H,9H)-dione;
[3a(4)Z,13aR]-6-methyl-9,10,11,12,13,13a,14,15-octahydro-17,1-(azenometheno)azeto[1,2-a]dipyrrolol[3,2-i:3′,4′-l][1,4,7]triazacyclopentadecine-3,8(2H,5H)-dione;
[16a(17)Z]-2,11-dimethyl-6,7,10,11-tetrahydro-1H,9H-12,14-(azenometheno)dipyrrolo[3,4-g:2′,3′-j][1,4,6,13]oxatriazacyclopentadecine-4,16(5H,15H)-dione;
[16a(17)Z]-2,5,11-trimethyl-6,7,10,11-tetrahydro-1H,9H-12,14-(azenometheno)dipyrrolo[3,2-f:3′,4′-i][1,4,13]oxadiazacyclopentadecine-4,16(5H,15H)-dione;
[17a(18)Z]-2,12-dimethyl-6,7,9,10,11,12-hexahydro-1H-13,15-(azenometheno)dipyrrolo[3,2-f:3′,4′-i][1,4,13]oxadiazacyclohexadecine-4,17(5H,16H)-dione;
[17a(18)Z]-2,5,12-trimethyl-6,7,9,10,11,12-hexahydro-1H-13,15-(azenometheno)dipyrrolo[3,2-f:3′,4′-i][1,4,13]oxadiazacyclohexadecine-4,17(5H,16H)-dione;
[17a(18)Z]-2,5,12-trimethyl-6,7,9,10,11,12-hexahydro-1H-13,15-(azenometheno)dipyrrolo[3,2-f:3′,4′-i][1,4,11,13]oxatriazacyclohexadecine-4,17(5H,16H)-dione;
[16a(17)Z]-2,5,11-trimethyl-6,7,8,9,10,11-hexahydro-1H-12,14-(azenometheno)dipyrrolo[3,2-i:3′,4′-l][1,4,7]triazacyclopentadecine-4,16(5H,15H)-dione;
[16a(17)Z]-2,5,11-trimethyl-6,7,8,9,10,11-hexahydro-1H-12,14-(ethanediylidene)dipyrrolo[3,2-k:3′,4′-n][1,3,6,9]tetraazacyclopentadecine-4,16(5H,15H)-dione;
[16a(17)Z]-2,5,11-trimethyl-6,7,8,9,10,11-hexahydro-1H-12,14-(azenometheno)dipyrrolo[3,2-k:3′,4′-n][1,3,6,9]tetraazacyclopentadecine-4,16(5H,15H)-dione;
[16a(17)Z]-11-cyclopropyl-2,5-dimethyl-6,7,8,9,10,11-hexahydro-1H-12,14-(ethanediylidene)dipyrrolo[3,2-k:3′,4′-n][1,3,6,9]tetraazacyclopentadecine-4,16(5H,15H)-dione;
[16a(17)Z]-11-cyclopropyl-2-methyl-6,7,8,9,10,11-hexahydro-1H-12,14-(azenometheno)dipyrrolo[3,2-k:3′,4′-n][1,3,6,9]tetraazacyclopentadecine-4,16(5H,15H)-dione;
[10R,16a(17)Z]-2,5,10-trimethyl-6,7,8,9,10,11-hexahydro-1H-12,14-(azenometheno)dipyrrolo[3,2-i:3′,4′-l][1,4,7]triazacyclopentadecine-4,16(5H,15H)-dione;
[10S,16a(17)Z]-2,5,10-trimethyl-6,7,8,9,10,11-hexahydro-1H-12,14-(azenometheno)dipyrrolo[3,2-i:3′,4′-l][1,4,7]triazacyclopentadecine-4,16(5H,15H)-dione;
[10S,16a(17)Z]-2,5,10-trimethyl-6,7,10,11-tetrahydro-1H,9H-12,14-(azenometheno)dipyrrolo[3,2-f:3′,4′-i][1,4,13]oxadiazacyclopentadecine-4,16(5H,15H)-dione;
[10S,16a(17)Z]-2,5,10-trimethyl-6,7,10,11-tetrahydro-1H,9H-12,14-(azenometheno)dipyrrolo[3,4-g:2′,3′-j][1,4,6,13]oxatriazacyclopentadecine-4,16(5H,15H)-dione;
[10S,16a(17)Z]-2,5,10-trimethyl-6,7,9,10-tetrahydro-1H-12,14-(azenometheno)dipyrrolo[3,2-i:3′,4′-l][1,4,7]dioxazacyclopentadecine-4,16(5H,15H)-dione;
[9S,16a(17)Z]-2,5,10-trimethyl-6,7,9,10-tetrahydro-1H-12,14-(azenometheno)dipyrrolo[3,4-d:2′,3′-g][1,13,3,10]dioxadiazacyclopentadecine-4,16(5H,15H)-dione;
[10S,16a(17)Z]-2,5,10-trimethyl-6,7,9,10-tetrahydro-1H-12,14-(ethanediylidene)dipyrrolo[3,4-d:2′,3′-g][1,13,3,10]dioxadiazacyclopentadecine-4,16(5H,15H)-dione;
[10S,16a(17)Z]-2,5,10-trimethyl-5,6,7,8,9,10-hexahydro-12,14-(azenometheno)dipyrrolo[3,4-d:2′,3′-g][1,3,10,13]oxatriazacyclopentadecine-4,16(1H,15H)-dione;
[10S,16a(17)Z]-2,5,10-trimethyl-5,6,7,8,9,10-hexahydro-12,14-(ethanediylidene)dipyrrolo[3,4-d:2′,3′-g][1,3,10,13]oxatriazacyclopentadecine-4,16(1H,15H)-dione;
[10S,16a(17)Z]-2,5,10-trimethyl-5,6,7,8,9,10-hexahydro-12,14-(azenometheno)dipyrrolo[3,2-i:3′,4′-l][1,4,7]oxadiazacyclopentadecine-4,16(1H,15H)-dione;
[9R,16a(17)Z]-2,5,9-trimethyl-6,7,9,10-tetrahydro-1H-12,14-(azenometheno)dipyrrolo[3,4-d:2′,3′-g][1,13,3,10]dioxadiazacyclopentadecine-4,16(5H,15H)-dione;
[9S,16a(17)Z]-2,5,9-trimethyl-6,7,9,10-tetrahydro-1H-12,14-(azenometheno)dipyrrolo[3,4-d:2′,3′-g][1,13,3,10]dioxadiazacyclopentadecine-4,16(5H,15H)-dione;
[16a(17)Z]-2,5-dimethyl-6,7,9,10-tetrahydro-1H-12,14-(ethanediylidene)dipyrrolo[3,2-i:3′,4′-1]l[1,4,7]dioxazacyclopentadecine-4,16(5H,15H)-dione;
[9S,16a(17)Z]-2,5,10-trimethyl-6,7,9,10-tetrahydro-1H-12,14-(ethanediylidene)dipyrrolo[3,2-i:3′,4′-][1,4,7]dioxazacyclopentadecine-4,16(5H,15H)-dione;
[9R,16a(17)Z]-2,5,10-trimethyl-6,7,9,10-tetrahydro-1H-12,14-(ethanediylidene)dipyrrolo[3,2-i:3′,4′-l][1,4,7]dioxazacyclopentadecine-4,16(5H,15H)-dione;
[9S,16a(17)Z]-2,10-dimethyl-6,7,9,10-tetrahydro-1H-12,14-(ethanediylidene)dipyrrolo[3,2-i:3′,4′-l][1,4,7]dioxazacyclopentadecine-4,16(5H,15H)-dione;
[10S,16a(17)Z]-2,5,10-trimethyl-5,6,7,8,9,10-hexahydro-12,14-(ethanediylidene)dipyrrolo[3,2-i:3′,4′-l][1,4,7]oxadiazacyclopentadecine-4,16(1H,15H)-dione;
[9R,16a(17)Z]-2,5,9-trimethyl-6,7,9,10-tetrahydro-1H-12,14-(ethanediylidene)dipyrrolo[3,2-i:3′,4′-l][1,4,7]dioxazacyclopentadecine-4,16(5H,15H)-dione;
[9S,16a(17)Z]-2,5,9-trimethyl-6,7,9,10-tetrahydro-1H-12,14-(ethanediylidene)dipyrrolo[3,2-i:3′,4′-l][1,4,7]dioxazacyclopentadecine-4,16(5H,15H)-dione;
[17a(18)Z]-2-methyl-6,7,10,11-tetrahydro-1H,9H-13,15-(ethanediylidene)dipyrrolo[3,2-f:3′,4′-i][1,13,4]oxathiazacyclohexadecine-4,17(5H,16H)-dione;
[17a(18)Z]-2-methyl-6,7,10,11-tetrahydro-1H-13,15-(ethanediylidene)-12λ⁶-dipyrrolo[3,2-f:3′,4′-i][1,13,4]oxathiazacyclohexadecine-4,12,12,17(5H,9H,16H)-tetrone;
[17a(18)Z]-2-methyl-6,7,9,10-tetrahydro-1H,12H-13,15-(ethanediylidene)dipyrrolo[3,2-i:3′,4′-l][1,4,7]dioxazacyclohexadecine-4,17(5H,16H)-dione;
[17a(18)Z]-2,12-dimethyl-6,7,9,10-tetrahydro-1H,12H-13,15-(ethanediylidene)dipyrrolo[3,2-i:3′,4′-l][1,4,7]dioxazacyclohexadecine-4,17(5H,16H)-dione;
[17a(18)Z]-2,12-dimethyl-6,7,9,10-tetrahydro-1H,12H-13,15-(ethanediylidene)dipyrrolo[3,2-i:3′,4′-l][1,4,7]dioxazacyclohexadecine-4,17(5H,16H)-dione;
[12S,17a(18)Z]-2,5,12-trimethyl-6,7,9,10-tetrahydro-1H,12H-13,15-(ethanediylidene)dipyrrolo[3,2-i:3′,4′-l][1,4,7]dioxazacyclohexadecine-4,17(5H,16H)-dione;
[17a(18)Z]-2,5-dimethyl-6,7,11,12-tetrahydro-1H-13,15-(ethanediylidene)dipyrrolo[3,2-ft3′,4′-i][1,4,14]oxadiazacyclohexadecine-4,10,17(5H,9H,16H)-trione;
[17a(18)Z]-2,5-dimethyl-6,7,11,12-tetrahydro-1H-13,15-(azenometheno)dipyrrolo[3,4-h:2′,3′-k][1,4,7,14]oxatriazacyclohexadecine-4,10,17(5H,9H,16H)-trione;
[17a(18)Z]-2,5-dimethyl-6,7,11,12-tetrahydro-1H-13,15-(azenometheno)dipyrrolo[3,2-f:3′,4′-i][1,4,14]oxadiazacyclohexadecine-4,10,17(5H,9H,16H)-trione;
[17a(18)Z]-2,5-dimethyl-6,7,11,12-tetrahydro-1H-13,15-(ethanediylidene)dipyrrolo[3,4-h:2′,3′-k][1,4,7,14]oxatriazacyclohexadecine-4,10,17(5H,9H,16H)-trione;
[17a(18)Z]-2,5,12-trimethyl-6,7,11,12-tetrahydro-1H-13,15-(ethanediylidene)dipyrrolo[3,4-h:2′,3′-k][1,4,7,14]oxatriazacyclohexadecine-4,10,17(5H,9H,16H)-trione;
[17a(18)Z]-2-methyl-6,7,11,12-tetrahydro-1H-13,15-(ethanediylidene)dipyrrolo[3,4-h:2′,3′-k][1,4,7,14]oxatriazacyclohexadecine-4,10,17(5H,9H,16H)-trione;
[17a(18)Z]-2,11-dimethyl-6,7,11,12-tetrahydro-1H-13,15-(ethanediylidene)dipyrrolo[3,4-h:2′,3′-k][1,4,7,14]oxatriazacyclohexadecine-4,10,17(5H,9H,16H)-trione;
[17a(18)Z]-2,11-dimethyl-6,7,11,12-tetrahydro-1H-13,15-(ethanediylidene)dipyrrolo[3,2-f:3′,4′-i][1,4,14]oxadiazacyclohexadecine-4,10,17(5H,9H,16H)-trione;
[17a(18)Z]-2,11-dimethyl-6,7,11,12-tetrahydro-1H-13,15-(azenometheno)dipyrrolo[3,4-h:2′,3′-k][1,4,7,14]oxatriazacyclohexadecine-4,10,17(5H,9H,16H)-trione;
[17a(18)Z]-2,11-dimethyl-6,7,11,12-tetrahydro-1H-13,15-(azenometheno)dipyrrolo[3,2-f:3′,4′-i][1,4,14]oxadiazacyclohexadecine-4,10,17(5H,9H,16H)-trione;
[18a(19)Z]-2-methyl-6,7,10,11-tetrahydro-1H,9H-14,16-(ethanediylidene)dipyrrolo[3,2-f:3′,4′-i][1,4,15]oxadiazacycloheptadecine-4,12,18(5H,13H,17H)-trione;
[18a(19)Z]-2,5-dimethyl-6,7,10,11-tetrahydro-1H,9H-14,16-(ethanediylidene)dipyrrolo[3,2-f:3′,4′-i][1,4,15]oxadiazacycloheptadecine-4,12,18(5H,13H,17H)-trione;
[18a(19)Z]-2,11-dimethyl-6,7,10,11-tetrahydro-1H,9H-14,16-(ethanediylidene)dipyrrolo[3,2-f:3′,4′-i][1,4,15]oxadiazacycloheptadecine-4,12,18(5H,13H,17H)-trione;
[13S,18a(19)Z]-2,13-dimethyl-6,7,10,11-tetrahydro-1H,9H-14,16-(ethanediylidene)dipyrrolo[3,2-f:3′,4′-i][1,4,15]oxadiazacycloheptadecine-4,12,18(5H,13H,17H)-trione;
[13R,18a(19)Z]-2,13-dimethyl-6,7,10,11-tetrahydro-1H,9H-14,16-(ethanediylidene)dipyrrolo[3,2-f:3′,4′-i][1,4,15]oxadiazacycloheptadecine-4,12,18(5H,13H,17H)-trione;
[18a(19)Z]-2-methyl-6,7,10,11-tetrahydro-1H,9H-14,16-(azenometheno)dipyrrolo[3,4-i:2′,3′-1][11,4,8,15]oxatriazacycloheptadecine-4,12,18(5H,13H,17H)-trione;
[13S,18a(19)Z]-2,13-dimethyl-6,7,10,11-tetrahydro-1H,9H-14,16-(ethanediylidene)dipyrrolo[3,4-i:2′,3′-l][1,4,8,15]oxatriazacycloheptadecine-4,12,18(5H,13H,17H)-trione;
[13S,18a(19)Z]-2,13-dimethyl-6,7,10,11-tetrahydro-1H,9H-14,16-(azenometheno)dipyrrolo[3,2-f:3′,4′-i][1,4,15]oxadiazacycloheptadecine-4,12,18(5H,13H,17H)-trione;
[13S,18a(19)Z]-13-hydroxy-2,13-dimethyl-6,7,10,11-tetrahydro-1H,9H-14,16-(ethanediylidene)dipyrrolo[3,2-f:3′,4′-i][1,4,15]oxadiazacycloheptadecine-4,12,18(5H,13H,17H)-trione;
[16a(17)Z]-2-methyl-6,7,9,10-tetrahydro-1H-12,14-(ethanediylidene)dipyrrolo[3,2-i:3′,4′-l][1,4,7]dioxazacyclopentadecine-4,16(5H,15H)-dione;
[16a(17)Z]-19-chloro-2-methyl-6,7,9,10-tetrahydro-1H-12,14-(ethanediylidene)dipyrrolo[3,2-i:3′,4′-l][1,4,7]dioxazacyclopentadecine-4,16(5H,15H)-dione;
[16a(17)Z]-19-chloro-2,5-dimethyl-6,7,9,10-tetrahydro-1H-12,14-(ethanediylidene)dipyrrolo[3,2-i:3′,4′-l][1,4,7]dioxazacyclopentadecine-4,16(5H,15H)-dione;
methyl[7R,16a(17)Z]-19-chloro-2,5-dimethyl-4,16-dioxo-4,5,6,7,9,10,15,16-octahydro-1H-12,14-(ethanediylidene)dipyrrolo[3,2-i:3′,4′-l][1,4,7]dioxazacyclopentadecine-7-carboxylate;
[7R,16a(17)Z]—N-(azetidin-3-yl)-19-chloro-2,5-dimethyl-4,16-dioxo-4,5,6,7,9,10,15,16-octahydro-1H-12,14-(ethanediylidene)dipyrrolo[3,2-i:3′,4′-l][1,4,7]dioxazacyclopentadecine-7-carboxamide;
[7R,16a(17)Z]-19-chloro-2,5-dimethyl-4,16-dioxo-N-(piperidin-4-yl)-4,5,6,7,9,10,15,16-octahydro-1H-12,14-(ethanediylidene)dipyrrolo[3,2-i:3′,4′-][1,4,7]dioxazacyclopentadecine-7-carboxamide;
[7R,16a(17)Z]-19-chloro-N,2,5-trimethyl-4,16-dioxo-4,5,6,7,9,10,15,16-octahydro-1H-12,14-(ethanediylidene)dipyrrolo[3,2-i:3′,4′-l][1,4,7]dioxazacyclopentadecine-7-carboxamide;
[7R,16a(17)Z]-19-chloro-2,5-dimethyl-4,16-dioxo-N-[(3R)-pyrrolidin-3-yl]-4,5,6,7,9,10,15,16-octahydro-1H-12,14-(ethanediylidene)dipyrrolo[3,2-i:3′,4′-l][1,4,7]dioxazacyclopentadecine-7-carboxamide;
[7R,16a(17)Z]-19-chloro-N,N,2,5-tetramethyl-4,16-dioxo-4,5,6,7,9,10,15,16-octahydro-1H-12,14-(ethanediylidene)dipyrrolo[3,2-i:3′,4′-l][1,4,7]dioxazacyclopentadecine-7-carboxamide;
[7R,16a(17)Z]-19-chloro-2,5-dimethyl-7-(4-methylpiperazine-1-carbonyl)-6,7,9,10-tetrahydro-1H-12,14-(ethanediylidene)dipyrrolo[3,2-i:3′,4′-][1,4,7]dioxazacyclopentadecine-4,16(5H,15H)-dione;
[10S,16a(17)Z]-2,5,10-trimethyl-6,7,9,10-tetrahydro-1H-12,14-(ethanediylidene)dipyrrolo[3,2-i:3′,4′-][1,4,7]dioxazacyclopentadecine-4,16(5H,15H)-dione;
[10S,16a(17)Z]-19-chloro-2,5,10-trimethyl-6,7,9,10-tetrahydro-1H-12,14-(ethanediylidene)dipyrrolo[3,2-i:3′,4′-][1,4,7]dioxazacyclopentadecine-4,16(5H,15H)-dione;
[16a(17)Z]-19-chloro-5-methyl-6,7,9,10-tetrahydro-1H-12,14-(ethanediylidene)dipyrrolo[3,2-i:3′,4′-][1,4,7]dioxazacyclopentadecine-4,16(5H,15H)-dione;
[16a(17)Z]-19-chloro-2,5-dimethyl-6,7,9,10-tetrahydro-1H-12,14-(ethanediylidene)dipyrrolo[3,2-i:3′,4′-l][1,4,7]oxathiazacyclopentadecine-4,16(5H,15H)-dione;
[16a(17)Z]-19-chloro-2,5-dimethyl-5,6,7,8,9,10-hexahydro-12,14-(ethanediylidene)dipyrrolo[3,2-i:3′,4′-][1,4,7]oxadiazacyclopentadecine-4,16(1H,15H)-dione;
[16a(17)Z]-19-chloro-2,5-dimethyl-6,7,9,10-tetrahydro-12,14-(ethanediylidene)-8λ⁶-dipyrrolo[3,2-i:3′,4′-l][1,4,7]oxathiazacyclopentadecine-4,8,8,16(1H,5H,15H)-tetrone;
[16a(17)Z]-19-chloro-2,5-dimethyl-6,7,9,10-tetrahydro-12,14-(ethanediylidene)-8λ⁴-dipyrrolo[3,2-i:3′,4′-l][1,4,7]oxathiazacyclopentadecine-4,8,16(1H,5H,15H)-trione;
[16a(17)Z]-19-chloro-5-methyl-6,7,9,10-tetrahydro-1H-12,14-(ethanediylidene)dipyrrolo[3,2-i:3′,4′-l][1,4,7]oxathiazacyclopentadecine-4,16(5H,15H)-dione;
[16a(17)Z]-2,5-dimethyl-6,7,9,10-tetrahydro-1H-12,14-(ethanediylidene)dipyrrolo[3,4-g:2′,3′-j][1,4,13]oxathiazacyclopentadecine-4,16(5H,15H)-dione;
[16a(17)Z]-2,5-dimethyl-6,7-dihydro-1H,9H-12,14-(ethanediylidene)-11λ⁶-dipyrrolo[3,4-g:2′,3′-j][1,4,13]oxathiazacyclopentadecine-4,11,11,16(5H,10H,15H)-tetrone;
[16a(17)Z]-5-methyl-6,7,9,10-tetrahydro-1H-12,14-(ethanediylidene)dipyrrolo[3,4-g:2′,3′-j][1,4,13]oxathiazacyclopentadecine-4,16(5H,15H)-dione;
[16a(17)Z]-19-chloro-5-methyl-6,7,9,10-tetrahydro-1H-12,14-(ethanediylidene)pyrazolo[4,3-i]pyrrolo[3,4-l][1,4,7]dioxazacyclopentadecine-4,16(5H,15H)-dione;
[16a(17)Z]-19-chloro-5-methyl-5,6,7,8,9,10-hexahydro-12,14-(ethanediylidene)dipyrrolo[3,2-i:3′,4′-l][1,4,7]oxadiazacyclopentadecine-4,16(1H,15H)-dione;
[16a(17)Z]-19-chloro-2,5,8-trimethyl-5,6,7,8,9,10-hexahydro-12,14-(ethanediylidene)dipyrrolo[3,2-i:3′,4′-l][1,4,7]oxadiazacyclopentadecine-4,16(1H,15H)-dione;
[16a(17)Z]-2,5-dimethyl-4,16-dioxo-4,5,6,7,9,10,15,16-octahydro-1H-12,14-(ethanediylidene)dipyrrolo[3,2-i:3′,4′-l][1,4,7]dioxazacyclopentadecine-19-carbonitrile;
[16a(17)Z]-19-chloro-2,5-dimethyl-6,7,9,10-tetrahydro-1H-12,14-(ethanediylidene)dipyrrolo[3,4-g:2′,3′-j][1,4,13]oxathiazacyclopentadecine-4,16(5H,15H)-dione;
[16a(17)Z]-19-chloro-5-methyl-6,7,9,10-tetrahydro-1H-12,14-(ethanediylidene)dipyrrolo[3,4-g:2′,3′-j][1,4,13]oxathiazacyclopentadecine-4,16(5H,15H)-dione; and
[16a(17)Z]-19-chloro-5-methyl-6,7-dihydro-1H,9H-12,14-(ethanediylidene)-11λ⁶-dipyrrolo[3,4-g:2′,3′-j][1,4,13]oxathiazacyclopentadecine-4,11,11,16(5H,10H,15H)-tetrone.

63. A pharmaceutical composition comprising a compound of claim 1, or a pharmaceutically acceptable salt thereof, and optionally one or more pharmaceutically acceptable excipients.

64. A method of treating disease, such as cancer, comprising administering to a subject in need of such treatment an effective amount of a compound of claim 1, or a pharmaceutically acceptable salt thereof.

65.-67. (canceled)
